SEQUENCE LISTING (106 sequences)

SEQ ID NO: 1 - protein, 10 aa, synthetic construct
GDSVSSNSAA

SEQ ID NO: 2 - protein, 9 aa, synthetic construct
TYYRSKWYN

SEQ ID NO: 3 - protein, 14 aa, synthetic construct
AREVTGDLEDAFDI

SEQ ID NO: 4 - protein, 6 aa, synthetic construct
QTIWSY

SEQ ID NO: 5 - skipped

SEQ ID NO: 6 - protein, 9 aa, synthetic construct
QQSYSIPQT

SEQ ID NO: 7 - protein, 8 aa, synthetic construct
GVSLPDYG

SEQ ID NO: 8 - protein, 7 aa, synthetic construct
IWGSETT

SEQ ID NO: 9 - protein, 14 aa, synthetic construct
AKHYYYGGSYAMDY

SEQ ID NO: 10 - protein, 6 aa, synthetic construct
QDISKY

SEQ ID NO: 11 - skipped

SEQ ID NO: 12 - protein, 9 aa, synthetic construct
QQGNTLPYT

SEQ ID NO: 13 - protein, 124 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 14 - protein, 107 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 15 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 16 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 17 - protein, 18 aa, synthetic construct
GSTSGSGKPGSGEGSTKG

SEQ ID NO: 18 - protein, 25 aa, synthetic construct
GGGGSGGGGSGGGGSGGGGSGGGGS

SEQ ID NO: 19 - protein, 5 aa, synthetic construct
GGGGS

SEQ ID NO: 20 - protein, 249 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 21 - protein, 236 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 22 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 23 - protein, 487 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSS

SEQ ID NO: 24 - protein, 506 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 25 - protein, 22 aa, synthetic construct
MLLLVTSLLLCELPHPAFLLIP

SEQ ID NO: 26 - protein, 24 aa, synthetic construct
IYIWAPLAGTCGVLLLSLVITLYC

SEQ ID NO: 27 - protein, 42 aa, synthetic construct
KRGRKKLLYIFKQPFMRPVQTTQEEDGCSCRFPEEEEGGCEL

SEQ ID NO: 28 - protein, 112 aa, synthetic construct
  VARIANT 14: note = Q can be replaced by K
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 711 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 729 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 2208 bp, synthetic construct
  source 1..2208: mol_type = other DNA
gccaccatgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatcccaggtgcagctgcagcagtctggacccggcctcgtgaagcctagccagaccctgtctctgacctgcgccatcagcggcgatagcgtgtccagcaatagcgccgcctggaactggatccggcagagcccttctagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggcagcacaagcggctctggcaagcctggatctggcgagggctctaccaagggcgatattcagatgacacagagcccctccagcctgtccgcctctgtgggagacagagtgacaatcacctgtcgggcctcccagaccatctggtcctatctgaattggtatcagcagcggcctggcaaggcccccaacctgctgatctatgccgccagctctctgcagtccggcgtgccatctagattcagcggcagaggcagcggcaccgatttcaccctgacaattagcagtctgcaggccgaggacttcgccacctactattgccagcagagctacagcatcccccagaccttcggccagggaacaaaactggaaatcaaagggggaggcggcagcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 32 - DNA, 2262 bp, synthetic construct
  source 1..2262: mol_type = other DNA
gccaccatgctgctgctcgtgacatctctgctgctgtgcgagctgccccaccccgcctttctgctgattcctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaagggcggagggggatctggcggcggaggatctgggggaggcggcagtgggggcggaggaagtggcgggggaggctctgatattcagatgacccagaccacctccagcctgtccgccagcctgggcgatcgcgtgaccatctcttgcagagccagccaggacatcagcaagtatctgaattggtatcagcagaaacccgacggcaccgtgaagctgctgatctaccacaccagcagactgcactccggcgtgccatccagattcagcggctctggctccggcaccgattatagcctgaccatcagcaacctggaacaggaagatatcgctacctacttttgtcagcaaggcaacaccctgccctacaccttcggcggaggcacaaaactggaaattaccggcagcaccagcggcagcggaaagcctggaagcggcgagggaagcaccaagggcgaagtgaaactgcaggaaagcggacccggactggtggccccaagccagtctctgagcgtgacatgtaccgtgtccggcgtgtccctgcccgactatggcgtgtcctggatcaggcagccccccagaaagggactggaatggctgggagtgatctggggcagcgagacaacctactacaacagcgccctgaagtccaggctgaccattatcaaggacaactccaagagccaggtgttcctgaagatgaacagcctgcagaccgacgacacagccatctactattgcgccaagcactactactacggcggcagctacgccatggactactggggacagggaacctccgtgaccgtgtcctctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 33 - protein, 45 aa, synthetic construct
TTTPAPRPPTPAPTIASQPLSLRPEACRPAAGGAVHTRGLDFACD

SEQ ID NO: 34 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 35 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 36 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 37 - DNA, 339 bp, synthetic construct
  source 1..339: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 38 - DNA, 672 bp, synthetic construct
  source 1..672: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 39 - protein, 711 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 729 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 232 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 8 aa, synthetic construct
GFTFTNHA

SEQ ID NO: 43 - protein, 8 aa, synthetic construct
ISGNGRTT

SEQ ID NO: 44 - protein, 11 aa, synthetic construct
AKDGGETLVDS

SEQ ID NO: 45 - protein, 8 aa, synthetic construct
GFTFSSHA

SEQ ID NO: 46 - protein, 8 aa, synthetic construct
ISGSGDFT

SEQ ID NO: 47 - protein, 12 aa, synthetic construct
AKDEDGGSLLGY

SEQ ID NO: 48 - protein, 8 aa, synthetic construct
GFTFSSYA

SEQ ID NO: 49 - protein, 8 aa, synthetic construct
ISGSGDYI

SEQ ID NO: 50 - protein, 14 aa, synthetic construct
AKEGTGANSSLADY

SEQ ID NO: 51 - protein, 8 aa, synthetic construct
GFTFSSHA

SEQ ID NO: 52 - protein, 8 aa, synthetic construct
ISGSGDYT

SEQ ID NO: 53 - protein, 12 aa, synthetic construct
AKDEDGGSLLGH

SEQ ID NO: 54 - protein, 118 aa, synthetic construct
QVQLVESGGGLVQPGGSLRLSCAASGFTFTNHAMSWVRQAPGKGLELVSSISGNGRTTYYADSVKGRFTISRDISKNTLDLQMNSLRAEDTAVYYCAKDGGETLVDSRGQGTLVTVSS

SEQ ID NO: 55 - protein, 119 aa, synthetic construct
QVQLVESGGGLVQPGGSLRLSCAASGFTFSSHAMTWVRQAPGKGLEWVAAISGSGDFTHYADSVKGRFTISRDNSKNTVSLQMNNLRAEDTAVYYCAKDEDGGSLLGYRGQGTLVTVSS

SEQ ID NO: 56 - protein, 121 aa, synthetic construct
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 57 - protein, 119 aa, synthetic construct
EVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSS

SEQ ID NO: 58 - protein, 117 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 59 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 60 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 61 - protein, 111 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 62 - protein, 111 aa, synthetic construct
DIVLTQSPASLAVSLGQRATISCRASESVDNYGFSFMHWYQQKPGQPPKLLIYRASNLESGIPARFSGSGSRTDFALTINPVETDDVATYYCQQSNKDPRTFGGGTKLEIK

SEQ ID NO: 63 - protein, 111 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 64 - protein, 246 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 65 - protein, 246 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 66 - protein, 246 aa, synthetic construct
DIVLTQSPASLAVSLGQRATISCRASESVDNYGFSFMHWYQQKPGQPPKLLIYRASNLESGIPARFSGSGSRTDFALTINPVETDDVATYYCQQSNKDPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 67 - protein, 246 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 68 - protein, 119 aa, synthetic construct
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 69 - protein, 118 aa, synthetic construct
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 70 - protein, 73 aa, synthetic construct
MGSWSEFWVRLGAIRERLDALGGSEAELAAFEKEIAAFESELQAYKGKGNPEVEKLRYTAATIRRFLQAYRHN

SEQ ID NO: 71 - protein, 375 aa, synthetic construct
MALPVTALLLPLALLLHAARPQVQLVESGGGLVQPGGSLRLSCAASGFTFTNHAMSWVRQAPGKGLELVSSISGNGRTTYYADSVKGRFTISRDISKNTLDLQMNSLRAEDTAVYYCAKDGGETLVDSRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 376 aa, synthetic construct
MALPVTALLLPLALLLHAARPQVQLVESGGGLVQPGGSLRLSCAASGFTFSSHAMTWVRQAPGKGLEWVAAISGSGDFTHYADSVKGRFTISRDNSKNTVSLQMNNLRAEDTAVYYCAKDEDGGSLLGYRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 378 aa, synthetic construct
MALPVTALLLPLALLLHAARPEVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 376 aa, synthetic construct
MALPVTALLLPLALLLHAARPEVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 376 aa, synthetic construct
MALPVTALLLPLALLLHAARPQVQLVESGGGLVQPGGSLRLSCAASGFTFTNHAMSWVRQAPGKGLELVSSISGNGRTTYYADSVKGRFTISRDISKNTLDLQMNSLRAEDTAVYYCAKDGGETLVDSRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 377 aa, synthetic construct
MALPVTALLLPLALLLHAARPQVQLVESGGGLVQPGGSLRLSCAASGFTFSSHAMTWVRQAPGKGLEWVAAISGSGDFTHYADSVKGRFTISRDNSKNTVSLQMNNLRAEDTAVYYCAKDEDGGSLLGYRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 379 aa, synthetic construct
MALPVTALLLPLALLLHAARPEVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 377 aa, synthetic construct
MALPVTALLLPLALLLHAARPEVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 372 aa, synthetic construct
MALPVTALLLPLALLLHAARPQVQLVESGGGLVQPGGSLRLSCAASGFTFTNHAMSWVRQAPGKGLELVSSISGNGRTTYYADSVKGRFTISRDISKNTLDLQMNSLRAEDTAVYYCAKDGGETLVDSRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 373 aa, synthetic construct
MALPVTALLLPLALLLHAARPQVQLVESGGGLVQPGGSLRLSCAASGFTFSSHAMTWVRQAPGKGLEWVAAISGSGDFTHYADSVKGRFTISRDNSKNTVSLQMNNLRAEDTAVYYCAKDEDGGSLLGYRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 375 aa, synthetic construct
MALPVTALLLPLALLLHAARPEVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 373 aa, synthetic construct
MALPVTALLLPLALLLHAARPEVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 503 aa, synthetic construct
MALPVTALLLPLALLLHAARPDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 503 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 503 aa, synthetic construct
MALPVTALLLPLALLLHAARPDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 507 aa, synthetic construct
MLLLVTSLLLCELPHPAFLLIPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 506 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 512 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 502 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 549 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 488 aa, synthetic construct
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 343 aa, synthetic construct
MAFLWLLSCWALLGTTFGDYKDDDDKGGGGSGGGGSMGSWSEFWVRLGAIRERLDALGGSEAELAAFEKEIAAFESELQAYKGKGNPEVEKLRYTAATIRRFLQAYRHNGGGGSGGGGSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 18 aa, synthetic construct
GSTSGSGKPGSGEGSTKG

SEQ ID NO: 94 - protein, 5 aa, synthetic construct
GGGGS

SEQ ID NO: 95 - protein, 21 aa, synthetic construct
MALPVTALLLPLALLLHAARP

SEQ ID NO: 96 - protein, 55 aa, synthetic construct
FVPVFLPAKPTTTPAPRPPTPAPTIASQPLSLRPEACRPAAGGAVHTRGLDFACD

SEQ ID NO: 97 - protein, 45 aa, synthetic construct; identical to SEQ ID NO: 33

SEQ ID NO: 98 - protein, 25 aa, synthetic construct
IYIWAPLAGTCGVLLLSLVITLYCN

SEQ ID NO: 99 - protein, 24 aa, synthetic construct
IYIWAPLAGTCGVLLLSLVITLYC

SEQ ID NO: 100 - protein, 47 aa, synthetic construct
RFSVVKRGRKKLLYIFKQPFMRPVQTTQEEDGCSCRFPEEEEGGCEL

SEQ ID NO: 101 - protein, 42 aa, synthetic construct; identical to SEQ ID NO: 27

SEQ ID NO: 102 - protein, 41 aa, synthetic construct
RSKRSRLLHSDYMNMTPRRPGPTRKHYQPYAPPRDFAAYRS

SEQ ID NO: 103 - protein, 37 aa, synthetic construct
RRDQRLPPDAHKPPGGGSFRTPIQEEQADAHSTLAKI

SEQ ID NO: 104 - protein, 112 aa, synthetic construct; identical to SEQ ID NO: 28

SEQ ID NO: 105 - protein, 5 aa, synthetic construct
  VARIANT 1..5: note = The entire sequence of amino acids 1-5 can be repeated up to nine times
GGGGS

SEQ ID NO: 106 - protein, 25 aa, synthetic construct
GGGGSGGGGSGGGGSGGGGSGGGGS

(19) United States
(12) Patent Application Publication
(10) Pub. No.: US 2026/0199468 A1
(43) Pub. Date: Jul. 16, 2026

(54) COMPOSITIONS AND METHODS FOR TREATING CANCER USING CELL THERAPIES

(71) Applicants: Orca Biosystems, Inc., Menlo Park, CA (US); The Board and Trustees of the Leland Stanford Junior University, Stanford, CA (US)

(72) Inventors: Nathaniel Fernhoff, Menlo Park, CA (US); Scott McClellan, Menlo Park, CA (US); Ivan Dimov, Menlo Park, CA (US); Lori Muffly, Palo Alto, CA (US); Melody Smith, Stanford, CA (US); David Miklos, Stanford, CA (US)

(21) Appl. No.: 19/558,231

(22) Filed: Mar. 5, 2026

Related U.S. Application Data
(63) Continuation of application No. PCT/US2024/045399, filed on Sep. 5, 2024.
(60) Provisional application No. 63/580,701, filed on Sep. 5, 2023.
(60) Provisional application No. 63/638,850, filed on Apr. 25, 2024.

(51) Int. Cl. A61K 40/31; A61K 31/255; A61K 31/436; A61K 31/675; A61K 31/7076; A61K 40/32; A61P 37/08

(52) U.S. Cl. CPC A61K 40/31; A61K 31/255; A61K 31/436; A61K 31/675; A61K 31/7076; A61K 40/32; A61P 37/08

(57) ABSTRACT

Various embodiments of the present disclosure provide therapeutic compositions and associated methods for a cellular therapy comprising one or more cells, such as allogeneic cells, comprising a chimeric receptor and improved hematopoietic stem cell transplantations, including methods to enhance protection from graft versus host disease while maintaining effective immune responses such as graft versus tumor immune responses.

### CROSS-REFERENCE TO RELATED APPLICATIONS

This application is a continuation of International Application Number PCT/US2024/045399, which claims the benefit of priority of U.S. Provisional Application Nos. 63/580,701, filed Sep. 5, 2023, and 63/638,850, filed Apr. 25, 2024, each of which is hereby incorporated by reference in their entirety for all purposes.

### SEQUENCE LISTING

The instant application contains a Sequence Listing which has been submitted electronically in XML format and is hereby incorporated by reference in its entirety. Said XML copy, created on 20 Dec. 2024, is named ORCA-023WO.XML and is 115,465 bytes in size.

### BACKGROUND

Patients with hematologic malignancies such as leukemia and lymphoma beyond first remission or with refractory relapse are rarely cured with standard chemotherapy. Myeloablative allogeneic hematopoietic cell transplantation (alloHCT) is associated with improved survival in these patients. Myeloablative alloHCT is a procedure in which the patient undergoes chemotherapy or radiation to ablate or destroy tissue in the bone causing the malignancy. They then receive hematopoietic cells, including hematopoietic stem and progenitor cells (HSPC) from a donor's blood. However, alloHCT has a major drawback in that it often results in graft versus host disease (GVHD). GVHD is a condition in which the transplanted donor peripheral blood stem cells view the patient's body as foreign, and the donated cells attack the patient's tissue (e.g., skin, GI tissue, liver tissue, and lung tissue) resulting in a number of complications, many of which can be serious and result in morbidity and mortality. Recent pre-clinical investigations using mouse models show that in CD19 positive lymphoma bearing BALB/c mice that were recipients of adoptive transfer of donor-derived 19-28z CAR T cells after a B6 allograft were less likely to succumb to GVHD compared control mice. Thus, there is a need for improved methods and therapies for administration allogeneic cells expressing a chimeric receptor with hematopoietic cell transplantation which have a reduced incidence and severity of GVHD including, reduced morbidity and mortality.

### BRIEF SUMMARY

Described herein, in certain embodiments, are methods of treating a human subject having or suspected of having a malignancy, comprising administering to the human subject a cellular therapy, wherein the cellular therapy comprises: (a) one or more allogeneic cells comprising a chimeric receptor; and (b) an immune-modulating cell therapy product, wherein the one or more allogeneic cells and/or one or more cells of the immune-modulating cell therapy product are from an HLA-compatible donor, relative to the human subject. In some embodiments, the one or more cells comprising the chimeric receptor comprise an isolated nucleic acid encoding the chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); and (b) one or more cells comprising a chimeric receptor or a pharmaceutical composition comprising an isolated nucleic acid encoding the chimeric receptor.

Described herein, in certain embodiments, are methods of treating a human subject having or suspected of having a hematologic malignancy, comprising administering to the human subject a multi-component cellular therapy, wherein the multi-component cellular therapy comprises a population of CD34⁺ hematopoietic stem and progenitor cells (HSPCs), and wherein the population of HSPCs comprises one or more cells comprising a chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD4⁺CD25⁺CD127^{dim} regulatory T cells (Tregs); and (b) one or more cells comprising a chimeric receptor or a pharmaceutical composition comprising an isolated nucleic acid encoding the chimeric receptor.

Described herein, in certain embodiments, are methods of treating a human subject having or suspected of having a hematologic malignancy, comprising administering to the human subject a multi-component cellular therapy, wherein the multi-component cellular therapy comprises a population of CD4⁺CD25⁺CD127^{dim} regulatory T cells (Tregs), and wherein the population of Tregs comprises one or more cells comprising a chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD45RA⁻ memory T cells (Tmems); and (b) one or more cells comprising a chimeric receptor or a pharmaceutical composition comprising an isolated nucleic acid encoding the chimeric receptor.

Described herein, in certain embodiments, are methods of treating a human subject having or suspected of having a hematologic malignancy, comprising administering to the human subject a multi-component cellular therapy, wherein the multi-component cellular therapy comprises a population of CD45RA⁻ memory T cells (Tmems), and wherein the population of Tmems comprises one or more cells comprising a chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); (b) a population of CD4⁺CD25⁺CD127^{dim} regulatory T cells (Tregs); and (c) one or more cells comprising a chimeric receptor or a pharmaceutical composition comprising an isolated nucleic acid encoding the chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); and (b) a population of CD4⁺CD25⁺CD127^{dim} regulatory T cells (Tregs), wherein the population of HSPCs, the population of Tregs, or the population of HSPCs and the population of Tregs comprises one or more cells comprising a chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); (b) a population of CD45RA⁻ memory T cells (Tmems); and (c) one or more cells comprising a chimeric receptor or a pharmaceutical composition comprising an isolated nucleic acid encoding the chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); and (b) a population of CD45RA⁻ memory T cells (Tmems), wherein the population of HSPCs, the population of Tmems, or the population of HSPCs and the population of Tmems comprises one or more cells comprising a chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); (b) a population of conventional CD3⁺ T cells (Tcons); and (c) one or more cells comprising a chimeric receptor or a pharmaceutical composition comprising an isolated nucleic acid encoding the chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); and (b) a population of conventional CD3⁺ T cells (Tcons), wherein the population of HSPCs, the population of Tcons, or the population of HSPCs and the population of Tcons comprises one or more cells comprising a chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); (b) a population of CD4⁺CD25⁺CD127^{dim} regulatory T cells (Tregs); (c) a population of conventional CD3⁺ T cells (Tcons); and (d) one or more cells comprising a chimeric receptor or a pharmaceutical composition comprising an isolated nucleic acid encoding the chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); (b) a population of CD4⁺CD25⁺CD127^{dim} regulatory T cells (Tregs); and (c) a population of conventional CD3⁺ T cells (Tcons), wherein the population of HSPCs, the population of Tregs, and/or the population of Tcons comprises one or more cells comprising a chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); (b) a population of CD4⁺CD25⁺CD127^{dim} regulatory T cells (Tregs); (c) a population of CD45RA⁻ memory T cells (Tmems); (d) a population of conventional CD3⁺ T cells (Tcons); and (e) one or more cells comprising a chimeric receptor or a pharmaceutical composition comprising an isolated nucleic acid encoding the chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); (b) a population of CD4⁺CD25⁺CD127^{dim} regulatory T cells (Tregs); (c) a population of CD45RA⁻ memory T cells (Tmems); and (c) a population of conventional CD3⁺ T cells (Tcons), wherein the population of HSPCs, the population of Tregs, the population of Tmems, and/or the population of Tcons comprises one or more cells comprising a chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs); (b) a population of CD4⁺CD25⁺CD127^{dim} regulatory T cells (Tregs); and (c) a population of conventional CD3⁺ T cells (Tcons), wherein the population of Tcons comprises one or more cells comprising a chimeric receptor.

Described herein, in certain embodiments, are methods of treating a human subject having or suspected of having a hematologic malignancy, comprising administering to the human subject a multi-component cellular therapy, wherein the multi-component cellular therapy comprises: (a) a population of CD34⁺ hematopoietic stem and progenitor cells (HSPCs), a population of CD4⁺CD25⁺CD127^{dim} regulatory T cells (Tregs), a population of conventional CD3⁺ T cells (Tcons), or any combination thereof; and (b) a population of tumor-infiltrating lymphocytes (TILs).

Described herein, in certain embodiments, are methods of treating a human subject having or suspected of having a hematologic condition, the method comprising: (a) administering a single conditioning regimen; and (b) administering two or more cell therapies.

Described herein, in certain embodiments, are multi-component cellular therapy products comprising: (a) one or more cells comprising a chimeric receptor or a pharmaceutical composition comprising an isolated nucleic acid encoding the chimeric receptor; and (b) an immune-modulating cell therapy product. In certain embodiments, the one or more cells comprising the chimeric receptor or the pharmaceutical composition and/or one or more cells of the immune-modulating cell therapy product are allogeneic cells from an HLA-compatible donor, relative to a human subject receiving the multi-component cellular therapy product.

Described herein, in certain embodiments, are multi-component cellular therapy products comprising: (a) one or more allogeneic cells comprising a chimeric receptor; and (b) an immune-modulating cell therapy product, wherein the one or more allogeneic cells and/or one or more cells of the immune-modulating cell therapy product are from an HLA-compatible donor, relative to a human subject receiving the multi-component cellular therapy product.

Described herein, in certain embodiments, are multi-component cellular therapy products comprising: (a) a population of CD34⁺ hematopoietic stem and progenitor cells (HSPCs); and (b) one or more cells comprising a chimeric receptor or a pharmaceutical composition comprising an isolated nucleic acid encoding the chimeric receptor.

Described herein, in certain embodiments, are multi-component cellular therapy products comprising a population of CD34⁺ hematopoietic stem and progenitor cells (HSPCs), wherein the population HSPCs comprises one or more cells comprising a chimeric receptor.

Described herein, in certain embodiments, are multi-component cellular therapy products comprising: (a) a population of CD4⁺CD25⁺CD127^{dim} regulatory T cells (Tregs); and (b) one or more cells comprising a chimeric receptor or a pharmaceutical composition comprising an isolated nucleic acid encoding the chimeric receptor.

Described herein, in certain embodiments, are multi-component cellular therapy products comprising a population of CD4⁺CD25⁺CD127^{dim} regulatory T cells (Tregs), wherein the population of Tregs comprises one or more cells comprising a chimeric receptor.

Described herein, in certain embodiments, are multi-component cellular therapy products comprising: (a) a population of CD45RA⁻ memory T cells (Tmems); and (b) one or more cells comprising a chimeric receptor or a pharmaceutical composition comprising an isolated nucleic acid encoding the chimeric receptor.

Described herein, in certain embodiments, are multi-component cellular therapy products comprising a population of CD45RA⁻ memory T cells (Tmems), wherein the population of Tmems comprises one or more cells comprising a chimeric receptor.

Described herein, in certain embodiments, are multi-component cellular therapy products comprising: (a) a population of CD34⁺ hematopoietic stem and progenitor cells (HSPCs); (b) a population of CD4⁺CD25⁺CD127^{dim} regulatory T cells (Tregs); and (c) one or more cells comprising a chimeric receptor or a pharmaceutical composition comprising an isolated nucleic acid encoding the chimeric receptor.

Described herein, in certain embodiments, are multi-component cellular therapy products comprising: (a) a population of CD34⁺ hematopoietic stem and progenitor cells (HSPCs); and (b) a population of CD4⁺CD25⁺CD127^{dim} regulatory T cells (Tregs), wherein the population of HSPCs, the population of Tregs, or the population of HSPCs and the population of Tregs comprises one or more cells comprising a chimeric receptor.

Described herein, in certain embodiments, are multi-component cellular therapy products comprising: (a) a population of CD34⁺ hematopoietic stem and progenitor cells (HSPCs); (b) a population of CD45RA⁻ memory T cells (Tmems); and (c) one or more cells comprising a chimeric receptor or a pharmaceutical composition comprising an isolated nucleic acid encoding the chimeric receptor.

Described herein, in certain embodiments, are multi-component cellular therapy products comprising: (a) a population of CD34⁺ hematopoietic stem and progenitor cells (HSPCs); and (b) a population of CD45RA⁻ memory T cells (Tmems); and wherein the population of HSPCs, the population of Tmems, or the population of HSPCs and the population of Tmems comprises one or more cells comprising a chimeric receptor.

Described herein, in certain embodiments, provide a multi-component cellular therapy products comprising: (a) a population of CD34⁺ hematopoietic stem and progenitor cells (HSPCs); (b) a population of conventional CD3⁺ T cells (Tcons); and (c) one or more cells comprising a chimeric receptor or a pharmaceutical composition comprising an isolated nucleic acid encoding the chimeric receptor.

Described herein, in certain embodiments, are multi-component cellular therapy products comprising: (a) a population of CD34⁺ hematopoietic stem and progenitor cells (HSPCs); and (b) a population of conventional CD3⁺ T cells (Tcons), wherein the population of HSPCs, the population of Tcons, or the population of HSPCs and the population of Tcons comprises one or more cells comprising a chimeric receptor.

Described herein, in certain embodiments, are multi-component cellular therapy products comprising: (a) a population of CD34⁺ hematopoietic stem and progenitor cells (HSPCs); (b) a population of CD4⁺CD25⁺CD127^{dim} regulatory T cells (Tregs); (c) a population of conventional CD3⁺ T cells (Tcons); and (d) one or more cells comprising a chimeric receptor or a pharmaceutical composition comprising an isolated nucleic acid encoding the chimeric receptor.

Described herein, in certain embodiments, are multi-component cellular therapy products comprising: (a) a population of CD34⁺ hematopoietic stem and progenitor cells (HSPCs); (b) a population of CD4⁺CD25⁺CD127^{dim} regulatory T cells (Tregs); and (c) a population of conventional CD3⁺ T cells (Tcons), wherein the population of HSPCs, the population of Tregs, and/or the population of Tcons comprises one or more cells comprising a chimeric receptor.

Described herein, in certain embodiments, are multi-component cellular therapy products comprising: (a) a population of CD34⁺ hematopoietic stem and progenitor cells (HSPCs); (b) a population of CD4⁺CD25⁺CD127^{dim} regulatory T cells (Tregs); (c) a population of CD45RA⁻ memory T cells (Tmems); (d) a population of conventional CD3⁺ T cells (Tcons); and (e) one or more cells comprising a chimeric receptor or a pharmaceutical composition comprising an isolated nucleic acid encoding the chimeric receptor.

Described herein, in certain embodiments, are multi-component cellular therapy products comprising: (a) a population of CD34⁺ hematopoietic stem and progenitor cells (HSPCs); (b) a population of CD4⁺CD25⁺CD127^{dim} regulatory T cells (Tregs); (c) a population of CD45RA⁻ memory T cells (Tmems); and (d) a population of conventional CD3⁺ T cells (Tcons), wherein the population of HSPCs, the population of Tregs, the population of Tmems, and/or the population of Tcons comprises one or more cells comprising a chimeric receptor.

Described herein, in certain embodiments, are multi-component cellular therapy products comprising: (a) a population of CD34⁺ hematopoietic stem and progenitor cells (HSPCs); (b) a population of CD4⁺CD25⁺CD127^{dim} regulatory T cells (Tregs); and (c) a population of conventional CD3⁺ T cells (Tcons), wherein the population of Tcons comprises one or more cells comprising a chimeric receptor.

Described herein, in certain embodiments, is a kit for use in preparation of a cellular therapy product, the kit comprising: (a) an anti-human CD34 affinity reagent; (b) an anti-human CD25 affinity reagent; (c) a vector comprising a nucleic acid encoding a chimeric receptor; and (d) a transfection reagent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 depicts a timeline schematic of the conditioning regiment and administration of the immune modulating cell therapy product concurrent with CAR T manufacturing prior to CAR T infusion.

FIG. 2 shows results of a flow cytometry assay assessing CD19 CAR expression.

FIG. 3 shows results of a CAR T cell cytotoxicity assay assessing percent of T cell killing.

FIG. 4A shows histograms of a CellTrace proliferation assay assessing CD3 T cell proliferation. FIG. 4B shows quantification of a CellTrace proliferation assay assessing CD3 T cell proliferation.

FIG. 5A shows histograms of a CellTrace proliferation assay assessing CAR T cell proliferation. FIG. 5B shows quantification of a CellTrace proliferation assay assessing CAR T cell proliferation.

FIG. 6 depicts baseline characteristics and clinical outcomes of patients treated with the immune modulating cell therapy product.

FIG. 7A shows the expansion and persistence of donor CD4⁺CD19/CD22 CAR T cells in patients treated with the immune modulating cell therapy product. FIG. 7B shows the expansion and persistence of donor CD8⁺CD19/CD22 CAR T cells in patients treated with the immune modulating cell therapy product. FIG. 7C shows the expansion and persistence of donor total CD19/CD22 CAR T cells in patients treated with the immune modulating cell therapy product.

FIG. 8 depicts qPCR results showing expansion and persistence of donor CD19/CD22 CAR T cells in patients treated with the immune modulating cell therapy product.

FIG. 9A shows percent whole blood donor chimerism in recipients of donor CD19/CD22 CAR T cells. FIG. 9B shows percent CD3⁺ cell donor chimerism in recipients of donor CD19/CD22 CAR T cells.

FIG. 10 depicts baseline characteristics and clinical outcomes of patients treated with the immune modulating cell therapy product.

### DETAILED DESCRIPTION

### Introduction

Described herein are multi-component cellular therapy products comprising: (a) one or more cells (e.g., allogeneic cells) comprising a chimeric receptor; and (b) an immune-modulating cell therapy product, wherein the one or more allogeneic cells and/or one or more cells of the immune-modulating cell therapy product are from an HLA-compatible donor, relative to a human subject receiving the multi-component cellular therapy product, and methods of using the multi-component cellular therapy products for treating human subjects having or suspected of having a malignancy. In some embodiments, the one or more cells comprising the chimeric receptor comprise a pharmaceutical composition comprising an isolated nucleic acid encoding the chimeric receptor.

Described herein are multi-component cellular therapy products comprising: (a) one or more cells (e.g., allogeneic cells) comprising a binding agent; and (b) an immune-modulating cell therapy product, wherein the one or more allogeneic cells and/or one or more cells of the immune-modulating cell therapy product are from an HLA-compatible donor, relative to a human subject receiving the multi-component cellular therapy product, and methods of using the multi-component cellular therapy products for treating human subjects having or suspected of having a malignancy. In some embodiments, the one or more cells comprising the chimeric receptor comprise a pharmaceutical composition comprising an isolated nucleic acid encoding the chimeric receptor.

Immune-modulating cell therapy products can include a regulatory T cell therapy, e.g., any of the regulatory T cell therapies described herein. Immune-modulating cell therapy products can include an allogeneic hematopoietic stem cell transplant (HSCT), e.g., any of the allogeneic HSCTs (alloHSCTs) described herein.

### Binding Agents, Chimeric Receptors, and Cells Including the Same

As provided herein, cells of the present disclosure may include one or more binding agents providing a means for binding to a target of interest, e.g. one or more epitopes of a target, such as the targets CD19, CD20, CD22, and/or BCMA. In certain embodiments, the means for binding to a target of interest (e.g., CD19, CD20, CD22, and/or BCMA) comprises an antibody or an antigen-binding fragment or equivalent thereof. In certain embodiments, the means for binding to a target of interest (e.g., CD19, CD20, CD22, and/or BCMA) is a binding agent comprising a T cell receptor (TCR) or a chimeric antigen receptor (CAR). In certain embodiments, the means for binding to a target of interest (e.g., CD19, CD20, CD22, and/or BCMA) is a binding agent comprising one or more CARs of the present disclosure that have antigenic specificity for CD19, CD20, CD22, and/or BCMA. In certain embodiments, the means for binding to a target of interest (e.g., CD19, CD20, CD22, and/or BCMA) is a binding agent comprising one or more of the heavy chain variable regions, the light chain variable regions, or their respective CDR1 regions, CDR2 regions, and CDR3 regions of the antigen binding domains provided in the present disclosure, e.g., the antigen binding domains that have antigenic specificity for CD19, CD20, CD22, and/or BCMA.

As provided herein, cells of the present disclosure may include one or more chimeric receptors. A chimeric receptor of the present disclosure may include, without limitation, a T cell receptor (TCR) or a chimeric antigen receptor (CAR).

In some embodiments, cells of the present disclosure that include a chimeric receptor may include a chimeric antigen receptor (CAR) that is an artificially constructed hybrid protein or polypeptide containing the antigen binding domains of one or more antibodies (e.g., single chain variable fragment (scFv)) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

The phrases “antigen(ic) specificity” and “elicit antigen-specific response,” as used herein, means that the CAR can specifically bind to and immunologically recognize antigen, such that binding of the CAR to the antigen elicits an immune response.

The phrase “dual specificity” and “dual specific,” as used herein, means that the same CAR can specifically bind to and immunologically recognize two different antigens, such that binding of the CAR to at least one of the two antigens elicits an immune response.

In some embodiments, CARs of the present disclosure may have antigenic specificity for CD19. In some embodiments, CARs of the present disclosure may have antigenic specificity for CD20. In some embodiments, CARs of the present disclosure may have antigenic specificity for CD22. In some embodiments, CARs of the present disclosure may have antigenic specificity for CD20 and CD19. In some embodiments, CARs of the present disclosure may have antigenic specificity for CD22 and CD19. In some embodiments, CARs of the present disclosure may have antigenic specificity for CD20 and CD22. In some embodiments, CARs of the present disclosure may have antigenic specificity for CD19, CD20 and CD22.

CD22 is a lineage-restricted B cell antigen belonging to the immunoglobulin (Ig) superfamily. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. Vaickus et al., Crit. Rev. Oncol./Hematol., 11:267-297 (1991); Bang et al., Clin. Cancer Res., 11:1545-50 (2005). CD19 (also known as B-lymphocyte antigen CD19, B4, and CVID3) is a cell surface molecule expressed only by B lymphocytes and follicular dendritic cells of the hematopoietic system. It is the earliest of the B-lineage-restricted antigens to be expressed and is present on most pre-B-cells and most non-T-cell acute lymphocytic leukemia cells and B-cell type chronic lymphocytic leukemia cells (Tedder and Isaacs, J. Immun., 143:712-717 (1989)). In other embodiments, CARs of the present disclosure may have antigenic specificity for BMCA.

The inventive dual specific CARs may provide many advantages. For example, the inventive dual specific CARs may, advantageously, provide a greater potency as compared to a CAR that has antigenic specificity for only one of CD19 and CD22 (but not both). The inventive dual specific CARs may also, advantageously, reduce or prevent cancer cell escape due to loss of expression of one of CD19 or CD22 by the cancer cell. For example, it is believed that the inventive dual specific CARs may reduce or prevent relapses that have been observed in cancer patients following treatment with a CAR having antigenic specificity for only CD19 and whose cancer has lost CD19 expression. The inventive dual specific CARs may also increase the patient population that may be successfully treated. For example, a patient that may fail to respond to a CAR therapy that targets only CD19 may respond to a CAR therapy that targets CD22, and a patient that may fail to respond to a CAR therapy that targets only CD22 may respond to a CAR therapy that targets CD19.

Without being bound to a particular theory or mechanism, it is believed that by eliciting an antigen-specific response against CD22 and CD19, the inventive CARs provide for one or more of any of the following: targeting and destroying CD22-expressing cancer cells, targeting and destroying CD19-expressing cancer cells, reducing or eliminating cancer cells, facilitating infiltration of immune cells to tumor site(s), and enhancing/extending anti-cancer responses.

An embodiment of the present disclosure provides a CAR comprising an anti-CD22 antigen binding domain of the m971 antibody (“m971”). The antigen binding domain of m971 specifically binds to CD22. In this regard, a preferred embodiment of the present disclosure provides CARs comprising an anti-CD22 antigen-binding domain comprising, consisting of, or consisting essentially of, a single chain variable fragment (scFv) of the antigen binding domain of m971. The HA22 immunotoxin and the m971 antibody bind to different CD22 epitopes.

The anti-CD22 antigen binding domain may comprise a light chain variable region and/or a heavy chain variable region. In an embodiment of the present disclosure, the heavy chain variable region comprises a CDR1 region, a CDR2 region, and a CDR3 region. In this regard, the anti-CD22 antigen binding domain may comprise one or more of a heavy chain CDR1 region comprising the amino acid sequence of SEQ ID NO: 1; a heavy chain CDR2 region comprising the amino acid sequence of SEQ ID NO: 2; and a heavy chain CDR3 region comprising the amino acid sequence of SEQ ID NO: 3. Preferably, the heavy chain of the anti-CD22 antigen binding domain comprises the amino acid sequences of all of SEQ ID NOs: 1-3.

In an embodiment of the present disclosure, the light chain variable region of the anti-CD22 antigen binding domain may comprise a light chain CDR1 region, a light chain CDR2 region, and a light chain CDR3 region. In this regard, the anti-CD22 antigen binding domain may comprise one or more of a light chain CDR1 region comprising the amino acid sequence of SEQ ID NO: 4; a light chain CDR2 region comprising the amino acid sequence of SEQ ID NO: 5; and a light chain CDR3 region comprising the amino acid sequence of SEQ ID NO: 6. Preferably, the light chain of the anti-CD22 antigen binding domain comprises the amino acid sequences of all of SEQ ID NOs: 4-6. In an especially preferred embodiment, the anti-CD22 antigen binding domain comprises the amino acid sequences of all of SEQ ID NO: 1-6.

The heavy chain variable region of the anti-CD22 antigen binding domain may comprise, consist of, or consist essentially of the amino acid sequence of SEQ ID NO: 13. The light chain variable region of the anti-CD22 antigen binding domain may comprise, consist of, or consist essentially of the amino acid sequence of SEQ ID NO: 14. Accordingly, in an embodiment of the present disclosure, the anti-CD22 antigen binding domain comprises a heavy chain variable region comprising the amino acid sequence of SEQ ID NO: 13 and/or a light chain variable region comprising the amino acid sequence of SEQ ID NO: 14. Preferably, the anti-CD22 antigen binding domain comprises the amino acid sequences of both SEQ ID NOs: 13 and 14.

An embodiment of the present disclosure provides a CAR comprising an anti-CD19 antigen binding domain of the FMC63 antibody (“FMC63”). The antigen binding domain of FMC63 specifically binds to CD19. In this regard, a preferred embodiment of the present disclosure provides CARs comprising an anti-CD19 antigen-binding domain comprising, consisting of, or consisting essentially of, a single chain variable fragment (scFv) of the antigen binding domain of FMC63.

The anti-CD19 antigen binding domain may comprise a light chain variable region and/or a heavy chain variable region. In an embodiment of the present disclosure, the heavy chain variable region of the anti-CD19 antigen binding domain comprises a CDR1 region, a CDR2 region, and a CDR3 region. In this regard, the anti-CD19 antigen binding domain may comprise one or more of a heavy chain CDR1 region comprising the amino acid sequence of SEQ ID NO: 7; a heavy chain CDR2 region comprising the amino acid sequence of SEQ ID NO: 8; and a heavy chain CDR3 region comprising the amino acid sequence of SEQ ID NO: 9. Preferably, the heavy chain of the anti-CD19 antigen binding domain comprises the amino acid sequences of all of SEQ ID NOs: 7-9.

In an embodiment of the present disclosure, the light chain variable region of the anti-CDl 9 antigen binding domain may comprise a light chain CDRl region, a light chain CDR2 region, and a light chain CDR3 region. In this regard, the anti-CDl 9 antigen binding domain may comprise one or more of a light chain CDRl region comprising the amino acid sequence of SEQ ID NO: 10; a light chain CDR2 region comprising the amino acid sequence of SEQ ID NO: 11; and a light chain CDR3 region comprising the amino acid sequence of SEQ ID NO: 12. Preferably, the light chain of the anti-CD19 antigen binding domain comprises the amino acid sequences of all of SEQ ID NOs: 10-12. In an especially preferred embodiment, the anti-CD19 antigen binding domain comprises the amino acid sequences of all of SEQ ID NO: 7-12.

The heavy chain variable region of the anti-CD19 antigen binding domain may comprise, consist of, or consist essentially of the amino acid sequence of SEQ ID NO: 15. The light chain variable region of the anti-CD19 antigen binding domain may comprise, consist of, or consist essentially of the amino acid sequence of SEQ ID NO: 16. Accordingly, in an embodiment of the present disclosure, the anti-CD19 antigen binding domain comprises a heavy chain variable region comprising the amino acid sequence of SEQ ID NO: 15 and/or a light chain variable region comprising the amino acid sequence of SEQ ID NO: 16. Preferably, the anti-CD19 antigen binding domain comprises the amino acid sequences of both SEQ ID NOs: 15 and 16.

In some embodiments of the present disclosure provides a CAR comprising an anti-CD20 antigen binding domain of the Leu16 antibody (“Leu16”). In some embodiments of the present disclosure provides a CAR comprising an anti-CD20 antigen binding domain of the 1F5 antibody (“1F5”). In some embodiments, a CAR of the present disclosure is C-CAR066.

The dual specific CARs have an anti-CD22 antigen binding domain and an anti-CD19 antigen binding domain. In an embodiment of the present disclosure, the CAR comprises all six CDR regions of both of the anti-CD22 antigen binding domain and the anti-CD19 antigen binding domain. In this regard, the CAR may comprise all of SEQ ID NOs: 1-12. In another embodiment of the present disclosure, the CAR comprises the light chain variable region and the heavy chain variable region of both of the anti-CD22 antigen binding domain and the anti-CD19 antigen binding domain. In this regard, the CAR may comprise all of SEQ ID NOs: 13-16.

In some embodiments, the sequences for the light and heavy chain CDR regions and light and heavy chain variable regions are selected from TABLE 1A. In some embodiments, CDRs can be annotated and/or defined in accordance with various CDR annotation schemes, such as the IMGT definition (see Lefranc, (1999) The Immunologist, 7, 132-136), the Kabat numbering scheme (see Kabat et al., 1992, Sequences of Proteins of Immunological Interest, DIANE Publishing: 2719), the Chothia numbering schemes (see Chothia and Lesk, J Mol Biol, 1987, 196:901-917), a combination of Kabat and Chothia. Other CDR annotation schemes include, but are not limited to, the AbM definition (see Whitelegg & Rees, Protein Eng. 2000, 13:819-824; Whitelegg & Rees, Methods Mol Biol. 2004, 248:51-91), and/or the contact definition (see MacCallum et al., J. Mol. Biol. 1996, 262:732-745). While CDR sequences provided herein may be according to one CDR annotation schemes (e.g., IMGT, Kabat and/or Chothia), one skilled in the art can recognize and determine CDR sequences based on other CDR annotation schemes through analysis and annotation of the light and heavy chain variable region sequences described herein.

TABLE 1A

| SEQ | | |
|---|---|---|
| ID | | |
| NO | Name | Sequence |
| 1 | CD22_HC-CDR_1 | GDSVSSNSAA |
| 2 | CD22_HC-CDR_2 | TYYRSKWYN |
| 3 | CD22_HC-CDR_3 | AREVTGDLEDAFDI |
| 4 | CD22_LC-CDR_1 | QTIWSY |
| 5 | CD22_LC-CDR_2 | AAS |
| 6 | CD22_LC-CDR_3 | QQSYSIPQT |
| 7 | CD19_HC-CDR_1 | GVSLPDYG |
| 8 | CD19_HC-CDR_2 | IWGSETT |
| 9 | CD19_HC-CDR_3 | AKHYYYGGSYAMDY |
| 10 | CD19_LC-CDR_1 | QDISKY |
| 11 | CD19_LC-CDR_2 | HTS |
| 12 | CD19_LC-CDR_3 | QQGNTLPYT |
| 13 | CD22 HCVR | QVQLQQSGPGLVKPSQTLSLTCAISGDSVSSNSAAWNWIRQSPSRGL |
| | | EWLGRTYYRSKWYNDYAVSVKSRITINPDTSKNQFSLQLNSVTPED |
| | | TAVYYCAREVTGDLEDAFDIWGQGTMVTVSS |
| 14 | CD22 LCVR | DIQMTQSPSSLSASVGDRVTITCRASQTIWSYLNWYQQRPGKAPNLL |
| | | IYAASSLQSGVPSRFSGRGSGTDFTLTISSLQAEDFATYYCQQSYSI |
| | | PQTFGQGTKLEIK |
| 15 | CD19 HCVR | EVKLQESGPGLVAPSQSLSVTCTVSGVSLPDYGVSWIRQPPRKGLEW |
| | | LGVIWGSETTYYNSALKSRLTIIKDNSKSQVFLKMNSLQTDDTAIYY |
| | | CAKHYYYGGSYAMDYWGQGTSVTVSS |
| 16 | CD19 LCVR | DIQMTQTTSSLSASLGDRVTISCRASQDISKYLNWYQQKPDGTVKLL |
| | | IYHTSRLHSGVPSRFSGSGSGTDYSLTISNLEQEDIATYFCQQGNTLP |
| | | YTFGGGTKLEIT |

The anti-CD22 antigen binding domain and the anti-CD19 antigen binding domain may comprise any antigen binding portion of the anti-CD22 or anti-CD19 antibody, respectively. The antigen binding portion can be any portion that has at least one antigen binding site, such as Fab, F(ab′)₂, dsFv, scFv, diabodies, and triabodies. Preferably, the antigen binding portion is a single-chain variable region fragment (scFv) antibody fragment. An scFv is a truncated Fab fragment including the variable (V) domain of an antibody heavy chain linked to a V domain of a light antibody chain via a synthetic peptide linker, which can be generated using routine recombinant DNA technology techniques. Similarly, disulfide-stabilized variable region fragments (dsFv) can be prepared by recombinant DNA technology.

In some embodiments of the present disclosure provides a CAR comprising an anti-CD22 antigen binding domain of the YK-CD22 antibody (“YK-CD22”). In some embodiments of the present disclosure provides a CAR comprising an anti-CD22 antigen binding domain of the m971 antibody (“m971”). In some embodiments of the present disclosure provides a CAR comprising an anti-CD22 antigen binding domain of the FH80 antibody (“FH80”).

In an embodiment of the present disclosure, the light chain variable region and the heavy chain variable region of the anti-CD22 antigen binding domain are joined to each other by a linker. The linker may comprise any suitable amino acid sequence. In an embodiment of the present disclosure, the linker is a Gly/Ser linker from about 1 to about 100, from about 3 to about 20, from about 5 to about 30, from about 5 to about 18, or from about 3 to about 8 amino acids in length and consists of glycine and/or serine residues in sequence. Accordingly, the Gly/Ser linker may consist of glycine and/or serine residues. In some embodiments, the Gly/Ser linker is a peptide of the formula: (Xaal)ₙ wherein each amino acid residue Xaal is selected independently from glycine and serine and n is an integer from 3 to 8. Preferably, the Gly/Ser linker comprises the amino acid sequence of SEQ ID NO: 19 (GGGGS). In another embodiment of the present disclosure, the linker comprises the amino acid sequence of SEQ ID NO: 17 (GSTSGSGKPGSGEGSTKG; also referred to as “218 linker”).

In an embodiment of the present disclosure, the light chain variable region and the heavy chain variable region of the anti-CD19 antigen binding domain are joined to each other by a linker. The linker may be any of the linkers described herein with respect to other aspects of the present disclosure. In an embodiment of the present disclosure, the light chain variable region and the heavy chain variable region of the anti-CD19 antigen binding domain are joined to each other by a linker comprising the amino acid sequence of SEQ ID NO: 17.

In an embodiment, the anti-CD22 antigen binding domain comprises a light chain variable region, a heavy chain variable region, and a linker. In this regard, an embodiment of the anti-CD22 antigen binding domain comprising a light chain variable region, a heavy chain variable region, and the 218 linker comprises, consists of, or consists essentially of, all of SEQ ID NOs: 1-6 and 17; all of SEQ ID NOs: 13, 14, and 17; or SEQ ID NO: 20 (QVQLQQSGPGLVKPSQTLSLTCAISGDSVSSNSAAWNWIRQSPSRGLEWLGRTYYRSKWYNDY AVSVKSRITINPDTSKNQFSLQLNSVTPEDTAVYYCAREVTGDLEDAFDIWGQGTMVTVSSGSTS GSGKPGSGEGSTKGDIQMTQSPSSLSASVGDRVTITCRASQTIWSYLNWYQQRPGKAPNLLIYAA SSLQSGVPSRFSGRGSGTDFTLTISSLQAEDFATYYCQQSYSIPQTFGQGTKLEIK). An embodiment of the anti-CD22 antigen binding domain comprising a light chain variable region, a heavy chain variable region, and a Gly/Ser linker comprises, consists of, or consists essentially of, all of SEQ ID NOs: 1-6 and 19; all of SEQ ID NOs: 13, 14, and 19; or SEQ ID NO: 21

(QVQLQQSGPGLVKPSQTLSLTCAISGDSVSSNSAAWNWIRQSPS
RGLEWLGRTYYRSKWYNDYAVSVKSRITINPDTSKNQFSLQLNSV
TPEDTAVYYCAREVTGDLEDAFDIWGQGTMVTVSSGGGGSDIQMT
QSPSSLSASVGDRVTITCRASQTIWSYLNWYQQRPGKAPNLLIYA
ASSLQSGVPSRFSGRGSGTDFTLTISSLQAEDFATYYCQQSYSIP
QTFGQGTKLEIK).

In an embodiment, the anti-CD19 antigen binding domain comprises a light chain variable region, a heavy chain variable region, and a linker. In this regard, an embodiment of the anti-CD19 antigen binding domain comprising a light chain variable region, a heavy chain variable region, and the 218 linker comprises, consists of, or consists essentially of, all of SEQ ID NOs: 7-12 and 17; all of SEQ ID NOs: 15-17; or SEQ ID NO: 22 (DIQMTQTTSSLSASLGDRVTISCRASQDISKYLNWYQQKPDGTVKLLIYHTSRLHSGVPSRFSGS GSGTDYSLTISNLEQEDIATYFCQQGNTLPYTFGGGTKLEITGSTSGSGKPGSGEGSTKGEVKLQE SGPGLVAPSQSLSVTCTVSGVSLPDYGVSWIRQPPRKGLEWLGVIWGSETTYYNSALKSRLTIIKD NSKSQVFLKMNSLQTDDTAIYYCAKHYYYGGSYAMDYWGQGTSVTVSS). An embodiment of the anti-CD19 antigen binding domain comprising a light chain variable region, a heavy chain variable region, and a Gly/Ser linker comprises, consists of, or consists essentially of, all of SEQ ID NOs: 7-12 and 19; all of SEQ ID NOs: 15, 16, and 19; or SEQ ID NO: 41

(DIQMTQTTSSLSASLGDRVTISCRASQDISKYLNWYQQKPDGTV
KLLIYHTSRLHSGVPSRFSGSGSGTDYSLTISNLEQEDIATYFCQ
QGNTLPYTFGGGTKLEITGGGGSEVKLQESGPGLVAPSQSLSVTC
TVSGVSLPDYGVSWIRQPPRKGLEWLGVIWGSETTYYNSALKSRL
TIIKDNSKSQVFLKMNSLQTDDTAIYYCAKHYYYGGSYAMDYWGQ
GTSVTVSS).

In an embodiment of the present disclosure, the anti-CD19 antigen binding domain and the anti-CD22 antigen binding domain are joined to each other by a linker. The linker may comprise any suitable amino acid sequence. The linker may, for example, comprise any of the linkers described herein with respect to other aspects of the present disclosure. Preferably, the linker joining the anti-CD19 antigen binding domain and the anti-CD22 antigen binding domain to each other comprises a Gly/Ser linker which is a peptide of the formula [GGGGS (SEQ ID NO: 105)]ₘ, wherein m is an integer from 1 to 10, from 2 to 8, or from 3 to 5. Preferably, m is 5. Preferably, the linker joining the anti-CD19 antigen binding domain and the anti-CD22 antigen binding domain to each other comprises the amino acid sequence of SEQ ID NO: 18 (GGGGSGGGGSGGGGSGGGGSGGGGS). In this regard, the anti-CD19 antigen binding domain and the anti-CD22 antigen binding domain that are joined to each other by a linker comprises the amino acid sequence of SEQ ID NO: 24 (comprising m971 heavy and light chains that are joined by a Gly/Ser linker and FMC63 heavy and light chains that are joined by the 218 linker, with the linker of SEQ ID NO: 18 positioned between the anti-CD22 and anti-CD19 antigen binding domains). While the anti-CD19 antigen binding domain may be positioned adjacent to the amino terminus of the anti-CD22 antigen binding domain (with a linker positioned between them), in an embodiment of the present disclosure, the anti-CD22 antigen binding domain is positioned adjacent to the amino terminus of the anti-CD19 antigen binding domain (with a linker positioned between them).

The heavy and light chains of the anti-CD19 and anti-CD22 antigen binding domains may be positioned in any suitable orientation with any of the linkers described herein positioned between the heavy and light chains. In an embodiment of the present disclosure, the CAR comprises (i) the FMC63 light chain positioned adjacent to the amino terminus of the m971 heavy chain with a linker positioned between them, (ii) the m971 heavy chain positioned adjacent to the amino terminus of the m971 light chain with a linker positioned between them, and (iii) the m971 light chain positioned adjacent to the amino terminus of the FMC63 heavy chain with a linker positioned between them (for example, SEQ ID NO: 23 and SEQ ID NO: 29 and 39, described in more detail below). In another embodiment of the present disclosure, the CAR comprises (i) the m971 heavy chain positioned adjacent to the amino terminus of the m971 light chain with a linker positioned between them, (ii) the m971 light chain positioned adjacent to the amino terminus of the FMC63 light chain with a linker positioned between them, and (iii) the FMC63 light chain positioned adjacent to the amino terminus of the FMC63 heavy chain with a linker positioned between them (for example, SEQ ID NO: 24 and SEQ ID NO: 30 and 40, described in more detail below).

In an embodiment, the antigen binding domain comprises a leader sequence. In an embodiment of the present disclosure, the leader sequence may be positioned at the amino terminus of the light chain variable region of the anti-CD19 antigen binding domain (e.g., at the amino terminus of the FMC63 light chain). In another embodiment of the present disclosure, the leader sequence is positioned at the amino terminus of the light chain variable region of the anti-CD22 antigen binding domain (e.g., at the amino terminus of the m971 heavy chain). The leader sequence may comprise any suitable leader sequence. In an embodiment, the leader sequence comprises, consists of, or consists essentially of the amino acid sequence of SEQ ID NO: 25 (MLLLVTSLLLCELPHPAFLLIP). In an embodiment of the present disclosure, while the leader sequence may facilitate expression of the CAR on the surface of the cell, the presence of the leader sequence in an expressed CAR is not necessary in order for the CAR to function. In an embodiment of the present disclosure, upon expression of the CAR on the cell surface, the leader sequence may be cleaved off of the CAR. Accordingly, in an embodiment of the present disclosure, the CAR lacks a leader sequence.

In an embodiment of the present disclosure, the CAR comprises a hinge domain. In an embodiment of the present disclosure, the hinge domain is a CDS hinge domain. In a preferred embodiment, the CDS hinge domain is human. Preferably, the CDS hinge domain comprises, consists of, or consists essentially of SEQ ID NO: 33

(TTTPAPRPPTPAPTIASQPLSLRPEACRPAAGGAVHTRGLDFACD).

In an embodiment of the present disclosure, the CAR comprises a transmembrane (TM) domain. In an embodiment of the present disclosure, the TM domain is a CDS TM domain. In a preferred embodiment, the CDS TM domain is human. Preferably, the CDS TM domain comprises, consists of, or consists essentially of SEQ ID NO: 26 (IYIWAPLAGTCGVLLLSLVITLYC).

In an embodiment of the present disclosure, the CAR comprises an intracellular T cell signaling domain. In an embodiment of the present disclosure, the intracellular T cell signaling domain comprises a 4-1BB intracellular T cell signaling sequence. 4-1BB, also known as CD137, transmits a potent costimulatory signal to T cells, promoting differentiation and enhancing long-term survival of T lymphocytes. Preferably, the 4-1BB intracellular T cell signaling sequence is human. In a preferred embodiment, the 4-1BB intracellular T cell signaling sequence comprises, consists of, or consists essentially of the amino acid sequence of SEQ ID NO: 27

(KRGRKKLLYIFKQPFMRPVQTTQEEDGCSCRFPEEEEGGCEL).

In an embodiment of the present disclosure, the intracellular T cell signaling domain comprises a CD3 zeta (ζ) intracellular T cell signaling sequence. CD3ζ associates with TCRs to produce a signal and contains immunoreceptor tyrosine-based activation motifs (ITAMs). Preferably, the CD3ζ intracellular T cell signaling sequence is human. In a preferred embodiment, the CD3ζ intracellular T cell signaling sequence comprises, consists of, or consists essentially of the amino acid sequence of SEQ ID NO: 28 (RVKFSRSADAPAYXQGQNQLYNELNLGRREEYDVLDKRRGRDPEMGGKPRRKNPQEGLYNEL QKDKMAEAYSEIGMKGERRRGKGHDGLYQGLSTATKDTYDALHMQALPPR), wherein X at position 14 is glutamine or lysine.

Additional embodiments of the present disclosure provide full-length CARs comprising, consisting of, or consisting essentially of any of, the amino acid sequences set forth in TABLE 1B.

TABLE 1B

| | Components | |
|---|---|---|
| | Sequence providing | |
| | the anti-CD19 and | Transmembrane |
| Full length | anti-CD22 antigen | and |
| CAR sequence | binding domains | Signaling Domains |
| SEQ ID NO: 29 | SEQ ID NO: 23 | Transmembrane |
| (DIQMTQTTSSLSASLGDRVT | (DIQMTQTTSSLSASLGDRVT | and Signaling |
| ISCRASQDISKYLNWYQQKP | ISCRASQDISKYLNWYQQKP | Domains |
| DGTVKLLIYHTSRLHSGVPS | DGTVKLLIYHTSRLHSGVPS | CDS hinge domain |
| RFSGSGSGTDYSLTISNLEQ | RFSGSGSGTDYSLTISNLEQ | (SEQ ID NO: 33), |
| EDIATYFCQQGNTLPYTFGG | EDIATYFCQQGNTLPYTFGG | CDS TM domain |
| GTKLEITGGGGSQVQLQQSG | GTKLEITGGGGSQVQLQQSG | (SEQ ID NO: 26), |
| PGLVKPSQTLSLTCAISGDS | PGLVKPSQTLSLTCAISGDS | 4-1BB |
| VSSNSAAWNWIRQSPSRGLE | VSSNSAAWNWIRQSPSRGLE | intracellular T cell |
| WLGRTYYRSKWYNDYAVSVK | WLGRTYYRSKWYNDYAVSVK | signaling |
| SRITINPDTSKNQFSLQLNS | SRITINPDTSKNQFSLQLNS | sequence (SEQ ID |
| VTPEDTAVYYCAREVTGDLE | VTPEDTAVYYCAREVTGDLE | NO: 27), and |
| DAFDIWGQGTMVTVSSGSTS | DAFDIWGQGTMVTVSSGSTS | CD3ζ |
| GSGKPGSGEGSTKGDIQMTQ | GSGKPGSGEGSTKGDIQMTQ | intracellular T |
| SPSSLSASVGDRVTITCRAS | SPSSLSASVGDRVTITCRAS | cell signaling |
| QTIWSYLNWYQQRPGKAPNL | QTIWSYLNWYQQRPGKAPNL | sequence (SEQ |
| LIYAASSLQSGVPSRFSGRG | LIYAASSLQSGVPSRFSGRG | ID NO: 28, |
| SGTDFTLTISSLQAEDFATY | SGTDFTLTISSLQAEDFATY | wherein X at |
| YCQQSYSIPQTFGQGTKLEI | YCQQSYSIPQTFGQGTKLEI | position 14 is |
| KGGGGSEVKLQESGPGLVAP | KGGGGSEVKLQESGPGLVAP | Lys) |
| SQSLSVTCTVSGVSLPDYGV | SQSLSVTCTVSGVSLPDYGV | |
| SWIRQPPRKGLEWLGVIWGS | SWIRQPPRKGLEWLGVIWGS | |
| ETTYYNSALKSRLTIIKDNS | ETTYYNSALKSRLTIIKDNS | |
| KSQVFLKMNSLQTDDTAIYY | KSQVFLKMNSLQTDDTAIYY | |
| CAKHYYYGGSYAMDYWGQGT | CAKHYYYGGSYAMDYWGQGT | |
| SVTVSSSGTTTPAPRPPTPA | SVTVSSS) | |
| PTIASQPLSLRPEACRPAAG | | |
| GAVHTRGLDFACDIYIWAPL | | |
| AGTCGVLLLSLVITLYCKRG | | |
| RKKLLYIFKQPFMRPVQTTQ | | |
| EEDGCSCRFPEEEEGGCELR | | |
| VKFSRSADAPAYKQGQNQLY | | |
| NELNLGRREEYDVLDKRRGR | | |
| DPEMGGKPRRKNPQEGLYNE | | |
| LQKDKMAEAYSEIGMKGERR | | |
| RGKGHDGLYQGLSTATKDTY | | |
| DALHMQALPPR) | | |
| SEQ ID NO: 30 | SEQ ID NO: 24 | CDS hinge domain |
| (QVQLQQSGPGLVKPSQTLSL | (QVQLQQSGPGLVKPSQTLSL | (SEQ ID NO: |
| TCAISGDSVSSNSAAWNWIR | TCAISGDSVSSNSAAWNWIR | 33), |
| QSPSRGLEWLGRTYYRSKWY | QSPSRGLEWLGRTYYRSKWY | CDS TM domain |
| NDYAVSVKSRITINPDTSKN | NDYAVSVKSRITINPDTSKN | (SEQ ID NO: |
| QFSLQLNSVTPEDTAVYYCA | QFSLQLNSVTPEDTAVYYCA | 26), |
| REVTGDLEDAFDIWGQGTMV | REVTGDLEDAFDIWGQGTMV | 4-1BB |
| TVSSGGGGSDIQMTQSPSSL | TVSSGGGGSDIQMTQSPSSL | intracellular T |
| SASVGDRVTITCRASQTIWS | SASVGDRVTITCRASQTIWS | cell signaling |
| YLNWYQQRPGKAPNLLIYAA | YLNWYQQRPGKAPNLLIYAA | sequence (SEQ |
| SSLQSGVPSRFSGRGSGTDF | SSLQSGVPSRFSGRGSGTDF | ID |
| TLTISSLQAEDFATYYCQQS | TLTISSLQAEDFATYYCQQS | NO: 27), and |
| YSIPQTFGQGTKLEIKGGGG | YSIPQTFGQGTKLEIKGGGG | CD3ζ |
| SGGGGSGGGGSGGGGSGGGG | SGGGGSGGGGSGGGGSGGGG | intracellular |
| SDIQMTQTTSSLSASLGDRV | SDIQMTQTTSSLSASLGDRV | T cell |
| TISCRASQDISKYLNWYQQK | TISCRASQDISKYLNWYQQK | signaling |
| PDGTVKLLIYHTSRLHSGVP | PDGTVKLLIYHTSRLHSGVP | sequence |
| SRFSGSGSGTDYSLTISNLE | SRFSGSGSGTDYSLTISNLE | (SEQ ID |
| QEDIATYFCQQGNTLPYTFG | QEDIATYFCQQGNTLPYTFG | NO: 28, |
| GGTKLEITGSTSGSGKPGSG | GGTKLEITGSTSGSGKPGSG | wherein X |
| EGSTKGEVKLQESGPGLVAP | EGSTKGEVKLQESGPGLVAP | at position |
| SQSLSVTCTVSGVSLPDYGV | SQSLSVTCTVSGVSLPDYGV | 14 is Lys) |
| SWIRQPPRKGLEWLGVIWGS | SWIRQPPRKGLEWLGVIWGS | |
| ETTYYNSALKSRLTIIKDNS | ETTYYNSALKSRLTIIKDNS | |
| KSQVFLKMNSLQTDDTAIYY | KSQVFLKMNSLQTDDTAIYY | |
| CAKHYYYGGSYAMDYWGQGT | CAKHYYYGGSYAMDYWGQGT | |
| SVTVSSTTTPAPRPPTPAPT | SVTVSS) | |
| IASQPLSLRPEACRPAAGGA | | |
| VHTRGLDFACDIYIWAPLAG | | |
| TCGVLLLSLVITLYCKRGRK | | |
| KLLYIFKQPFMRPVQTTQEE | | |
| DGCSCRFPEEEEGGCELRVK | | |
| FSRSADAPAYKQGQNQLYNE | | |
| LNLGRREEYDVLDKRRGRDP | | |
| EMGGKPRRKNPQEGLYNELQ | | |
| KDKMAEAYSEIGMKGERRRG | | |
| KGHDGLYQGLSTATKDTYDA | | |
| LHMQALPPR) | | |
| SEQ ID NO: 39 | SEQ ID NO: 23 | CDS hinge domain |
| (DIQMTQTTSSLSASLGDRVT | (DIQMTQTTSSLSASLGDRVT | (SEQ ID NO: |
| ISCRASQDISKYLNWYQQKP | ISCRASQDISKYLNWYQQKP | 33), |
| DGTVKLLIYHTSRLHSGVPS | DGTVKLLIYHTSRLHSGVPS | CDS TM domain |
| RFSGSGSGTDYSLTISNLEQ | RFSGSGSGTDYSLTISNLEQ | (SEQ ID NO: |
| EDIATYFCQQGNTLPYTFGG | EDIATYFCQQGNTLPYTFGG | 26), |
| GTKLEITGGGGSQVQLQQSG | GTKLEITGGGGSQVQLQQSG | 4-1BB |
| PGLVKPSQTLSLTCAISGDS | PGLVKPSQTLSLTCAISGDS | intracellular |
| VSSNSAAWNWIRQSPSRGLE | VSSNSAAWNWIRQSPSRGLE | T cell |
| WLGRTYYRSKWYNDYAVSVK | WLGRTYYRSKWYNDYAVSVK | signaling |
| SRITINPDTSKNQFSLQLNS | SRITINPDTSKNQFSLQLNS | sequence |
| VTPEDTAVYYCAREVTGDLE | VTPEDTAVYYCAREVTGDLE | (SEQ ID NO: |
| DAFDIWGQGTMVTVSSGSTS | DAFDIWGQGTMVTVSSGSTS | 27), and |
| GSGKPGSGEGSTKGDIQMTQ | GSGKPGSGEGSTKGDIQMTQ | CD3ζ |
| SPSSLSASVGDRVTITCRAS | SPSSLSASVGDRVTITCRAS | intracellular |
| QTIWSYLNWYQQRPGKAPNL | QTIWSYLNWYQQRPGKAPNL | T cell |
| LIYAASSLQSGVPSRFSGRG | LIYAASSLQSGVPSRFSGRG | signaling |
| SGTDFTLTISSLQAEDFATY | SGTDFTLTISSLQAEDFATY | sequence |
| YCQQSYSIPQTFGQGTKLEI | YCQQSYSIPQTFGQGTKLEI | (SEQ ID |
| KGGGGSEVKLQESGPGLVAP | KGGGGSEVKLQESGPGLVAP | NO: 28, |
| SQSLSVTCTVSGVSLPDYGV | SQSLSVTCTVSGVSLPDYGV | wherein X |
| SWIRQPPRKGLEWLGVIWGS | SWIRQPPRKGLEWLGVIWGS | at position |
| ETTYYNSALKSRLTIIKDNS | ETTYYNSALKSRLTIIKDNS | 14 is Gln) |
| KSQVFLKMNSLQTDDTAIYY | KSQVFLKMNSLQTDDTAIYY | |
| CAKHYYYGGSYAMDYWGQGT | CAKHYYYGGSYAMDYWGQGT | |
| SVTVSSSGTTTPAPRPPTPA | SVTVSSS) | |
| PTIASQPLSLRPEACRPAAG | | |
| GAVHTRGLDFACDIYIWAPL | | |
| AGTCGVLLLSLVITLYCKRG | | |
| RKKLLYIFKQPFMRPVQTTQ | | |
| EEDGCSCRFPEEEEGGCELR | | |
| VKFSRSADAPAYQQGQNQLY | | |
| NELNLGRREEYDVLDKRRGR | | |
| DPEMGGKPRRKNPQEGLYNE | | |
| LQKDKMAEAYSEIGMKGERR | | |
| RGKGHDGLYQGLSTATKDTY | | |
| DALHMQALPPR) | | |
| SEQ ID NO: 40 | SEQ ID NO: 24 | CDS hinge |
| KQVQLQQSGPGLVKPSQTLS | (QVQLQQSGPGLVKPSQTLSL | domain (SEQ |
| LTCAISGDSVSSNSAAWNWI | TCAISGDSVSSNSAAWNWIR | ID NO: 33), |
| RQSPSRGLEWLGRTYYRSKW | QSPSRGLEWLGRTYYRSKWY | CDS TM domain |
| YNDYAVSVKSRITINPDTSK | NDYAVSVKSRITINPDTSKN | (SEQ ID NO: |
| NQFSLQLNSVTPEDTAVYYC | QFSLQLNSVTPEDTAVYYCA | 26), |
| AREVTGDLEDAFDIWGQGTM | REVTGDLEDAFDIWGQGTMV | 4-1BB |
| VTVSSGGGGSDIQMTQSPSS | TVSSGGGGSDIQMTQSPSSL | intracellular |
| LSASVGDRVTITCRASQTIW | SASVGDRVTITCRASQTIWS | T cell |
| SYLNWYQQRPGKAPNLLIYA | YLNWYQQRPGKAPNLLIYAA | signaling |
| ASSLQSGVPSRFSGRGSGTD | SSLQSGVPSRFSGRGSGTDF | sequence |
| FTLTISSLQAEDFATYYCQQ | TLTISSLQAEDFATYYCQQS | (SEQ ID NO: |
| SYSIPQTFGQGTKLEIKGGG | YSIPQTFGQGTKLEIKGGGG | 27), and |
| GSGGGGSGGGGSGGGGSGGG | SGGGGSGGGGSGGGGSGGGG | CD3ζ |
| GSDIQMTQTTSSLSASLGDR | SDIQMTQTTSSLSASLGDRV | intracellular |
| VTISCRASQDISKYLNWYQQ | TISCRASQDISKYLNWYQQK | T cell |
| KPDGTVKLLIYHTSRLHSGV | PDGTVKLLIYHTSRLHSGVP | signaling |
| PSRFSGSGSGTDYSLTISNL | SRFSGSGSGTDYSLTISNLE | sequence |
| EQEDIATYFCQQGNTLPYTF | QEDIATYFCQQGNTLPYTFG | (SEQ ID |
| GGGTKLEITGSTSGSGKPGS | GGTKLEITGSTSGSGKPGSG | NO: 28, |
| GEGSTKGEVKLQESGPGLVA | EGSTKGEVKLQESGPGLVAP | wherein X |
| PSQSLSVTCTVSGVSLPDYG | SQSLSVTCTVSGVSLPDYGV | at position |
| VSWIRQPPRKGLEWLGVIWG | SWIRQPPRKGLEWLGVIWGS | 14 is Gln) |
| SETTYYNSALKSRLTIIKDN | ETTYYNSALKSRLTIIKDNS | |
| SKSQVFLKMNSLQTDDTAIY | KSQVFLKMNSLQTDDTAIYY | |
| YCAKHYYYGGSYAMDYWGQG | CAKHYYYGGSYAMDYWGQGT | |
| TSVTVSSTTTPAPRPPTPAP | SVTVSS) | |
| TIASQPLSLRPEACRPAAGG | | |
| AVHTRGLDFACDIYIWAPLA | | |
| GTCGVLLLSLVITLYCKRGR | | |
| KKLLYIFKQPFMRPVQTTQE | | |
| EDGCSCRFPEEEEGGCELRV | | |
| KFSRSADAPAYQQGQNQLYN | | |
| ELNLGRREEYDVLDKRRGRD | | |
| PEMGGKPRRKNPQEGLYNEL | | |
| QKDKMAEAYSEIGMKGERRR | | |
| GKGHDGLYQGLSTATKDTYD | | |
| ALHMQALPPR) | | |

In some embodiments, a CAR of the present disclosure may be tisagenlecleucel, axicabtagene ciloleucel, brexucabtagene autoleucel, or lisocabtagene maraleucel.

In some embodiments, CARs of the present disclosure may have antigenic specificity for B-cell maturation antigen (BCMA).

BCMA-specific CARs of the present disclosure may have a BCMA antigen binding domain. In some embodiments, the BCMA antigen binding domain is a single-domain antigen-binding domain having a heavy chain variable (VH) region comprising the CDR regions of SEQ ID NOs: 42-44, 54-47, 48-50, or 51-53. In another embodiment of the present disclosure, the CAR comprises the heavy chain variable region of the BCMA single domain antigen binding domain. In this regard, the CAR may comprise any of SEQ ID NOs: 54-57.

In some embodiments of the present disclosure, a CAR comprising a BCMA antigen binding domain comprises a single chain variable fragment (scFv) having a heavy chain variable (VH) region comprising the amino acid sequence of SEQ ID NO: 58 or 60 and a light chain variable (VL) region comprising the amino acid sequence of any of SEQ ID NOs: 59 and 61-63. In some embodiments, a BCMA CAR of the present disclosure comprises an scFv having any of the amino acid sequences of SEQ ID NOs: 64-67.

In some embodiments, a CAR of the present disclosure comprises a BCMA antigen binding that is a VHH antibody binding domain. In some embodiments, the VHH BCMA antigen binding domain comprises the amino acid sequence of SEQ ID NO: 68 or 69.

In some embodiments, a CAR of the present disclosure comprises a BCMA antigen binding comprising the amino acid sequence of SEQ ID NO: 70.

In some embodiments, the sequences for the BCMA antigen binding domain are selected from TABLE 2A. In some embodiments, CDRs can be annotated and/or defined in accordance with various CDR annotation schemes, such as the IMGT definition (see Lefranc, (1999) The Immunologist, 7, 132-136), the Kabat numbering scheme (see Kabat et al., 1992, Sequences of Proteins of Immunological Interest, DIANE Publishing: 2719), the Chothia numbering schemes (see Chothia and Lesk, J Mol Biol, 1987, 196:901-917), a combination of Kabat and Chothia. Other CDR annotation schemes include, but are not limited to, the AbM definition (see Whitelegg & Rees, Protein Eng. 2000, 13:819-824; Whitelegg & Rees, Methods Mol Biol. 2004, 248:51-91), and/or the contact definition (see MacCallum et al., J. Mol. Biol. 1996, 262:732-745). While CDR sequences provided herein may be according to one CDR annotation schemes (e.g., IMGT, Kabat and/or Chothia), one skilled in the art can recognize and determine CDR sequences based on other CDR annotation schemes through analysis and annotation of the light and heavy chain variable region sequences described herein.

TABLE 2A

| Antigen Binding | | SEQ ID |
|---|---|---|
| Domain | Amino Acid Sequence | NO: |
| BCMA sdHCVR1 | GFTFTNHA | 42 |
| CDR1 | | |
| BCMA sdHCVR1 | ISGNGRTT | 43 |
| CDR2 | | |
| BCMA sdHCVR1 | AKDGGETLVDS | 44 |
| CDR3 | | |
| BCMA sdHCVR2 | GFTFSSHA | 45 |
| CDR1 | | |
| BCMA sdHCVR2 | ISGSGDFT | 46 |
| CDR2 | | |
| BCMA sdHCVR2 | AKDEDGGSLLGY | 47 |
| CDR3 | | |
| BCMA sdHCVR3 | GFTFSSYA | 48 |
| CDR1 | | |
| BCMA sdHCVR3 | ISGSGDYI | 49 |
| CDR2 | | |
| BCMA sdHCVR3 | AKEGTGANSSLADY | 50 |
| CDR3 | | |
| BCMA sdHCVR4 | GFTFSSHA | 51 |
| CDR1 | | |
| BCMA sdHCVR4 | ISGSGDYT | 52 |
| CDR2 | | |
| BCMA sdHCVR4 | AKDEDGGSLLGH | 53 |
| CDR3 | | |
| BCMA HCVR1 | QVQLVESGGGLVQPGGSLRLSCAASGFTFTNHAMSWVRQAPGK | 54 |
| | GLELVSSISGNGRTTYYADSVKGRFTISRDISKNTLDLQMNSLRAE | |
| | DTAVYYCAKDGGETLVDSRGQGTLVTVSS | |
| BCMA HCVR2 | QVQLVESGGGLVQPGGSLRLSCAASGFTFSSHAMTWVRQAPGK | 55 |
| | GLEWVAAISGSGDFTHYADSVKGRFTISRDNSKNTVSLQMNNLR | |
| | AEDTAVYYCAKDEDGGSLLGYRGQGTLVTVSS | |
| BCMA HCVR3 | EVQLLESGGGLVQPGGSLRLSCAASGFTFSSYAMSWVRQAPGKG | 56 |
| | LEWVSSISGSGDYIYYADSVKGRFTISRDISKNTLYLQMNSLRAED | |
| | TAVYYCAKEGTGANSSLADYRGQGTLVTVSS | |
| BCMA HCVR4 | EVQLLESGGGLIQPGGSLRLSCAASGFTFSSHAMTWVRQAPGKG | 57 |
| | LEWVSAISGSGDYTHYADSVKGRFTISRDNSKNTVYLQMNSLRA | |
| | EDSAVYYCAKDEDGGSLLGHRGQGTLVTVSS | |
| BCMA scFv VH1 | QIQLVQSGPELKKPGETVKISCKASGYTFRHYSMNWVKQAPGKG | 58 |
| | LKWMGRINTESGVPIYADDFKGRFAFSVETSASTAYLVINNLKDE | |
| | DTASYFCSNDYLYSLDFWGQGTALTVSS | |
| BCMA scFv VL1 | DIVLTQSPPSLAMSLGKRATISCRASESVTILGSHLIYWYQQKPGQ | 59 |
| | PPTLLIQLASNVQTGVPARFSGSGSRTDFTLTIDPVEEDDVAVYYC | |
| | LQSRTIPRTFGGGTKLEIK | |
| BCMA scFv VH2 | QIQLVQSGPELKKPGETVKISCKASGYTFTDYSINWVKRAPGKGL | 60 |
| | KWMGWINTETREPAYAYDFRGRFAFSLETSASTAYLQINNLKYE | |
| | DTATYFCALDYSYAMDYWGQGTSVTVSS | |
| BCMA scFv VL2a | DIVLTQSPPSLAMSLGKRATISCRASESVTILGSHLIHWYQQKPGQ | 61 |
| | PPTLLIQLASNVQTGVPARFSGSGSRTDFTLTIDPVEEDDVAVYYC | |
| | LQSRTIPRTFGGGTKLEIK | |
| BCMA scFv VL2b | DIVLTQSPASLAVSLGQRATISCRASESVDNYGFSFMHWYQQKPG | 62 |
| | QPPKLLIYRASNLESGIPARFSGSGSRTDFALTINPVETDDVATYY | |
| | CQQSNKDPRTFGGGTKLEIK | |
| BCMA scFv VL2c | DIVLTQSPASLAMSLGKRATISCRASESVSVIGAHLIHWYQQKPG | 63 |
| | QPPKLLIYLASNLETGVPARFSGSGSGTDFTLTIDPVEEDDVAIYSC | |
| | LQSRIFPRTFGGGTKLEIK | |
| BCMA scFv1 | DIVLTQSPPSLAMSLGKRATISCRASESVTILGSHLIYWYQQKPGQ | 64 |
| | PPTLLIQLASNVQTGVPARFSGSGSRTDFTLTIDPVEEDDVAVYYC | |
| | LQSRTIPRTFGGGTKLEIKGSTSGSGKPGSGEGSTKGQIQLVQSGP | |
| | ELKKPGETVKISCKASGYTFRHYSMNWVKQAPGKGLKWMGRIN | |
| | TESGVPIYADDFKGRFAFSVETSASTAYLVINNLKDEDTASYFCSN | |
| | DYLYSLDFWGQGTALTVSS | |
| BCMA scFv2a | DIVLTQSPPSLAMSLGKRATISCRASESVTILGSHLIHWYQQKPGQ | 65 |
| | PPTLLIQLASNVQTGVPARFSGSGSRTDFTLTIDPVEEDDVAVYYC | |
| | LQSRTIPRTFGGGTKLEIKGSTSGSGKPGSGEGSTKGQIQLVQSGP | |
| | ELKKPGETVKISCKASGYTFTDYSINWVKRAPGKGLKWMGWINT | |
| | ETREPAYAYDFRGRFAFSLETSASTAYLQINNLKYEDTATYFCAL | |
| | DYSYAMDYWGQGTSVTVSS | |
| BCMA scFv2b | DIVLTQSPASLAVSLGQRATISCRASESVDNYGFSFMHWYQQKPG | 66 |
| | QPPKLLIYRASNLESGIPARFSGSGSRTDFALTINPVETDDVATYY | |
| | CQQSNKDPRTFGGGTKLEIKGSTSGSGKPGSGEGSTKGQIQLVQS | |
| | GPELKKPGETVKISCKASGYTFTDYSINWVKRAPGKGLKWMGWI | |
| | NTETREPAYAYDFRGRFAFSLETSASTAYLQINNLKYEDTATYFC | |
| | ALDYSYAMDYWGQGTSVTVSS | |
| BCMA scFv2c | DIVLTQSPASLAMSLGKRATISCRASESVSVIGAHLIHWYQQKPG | 67 |
| | QPPKLLIYLASNLETGVPARFSGSGSGTDFTLTIDPVEEDDVAIYSC | |
| | LQSRIFPRTFGGGTKLEIKGSTSGSGKPGSGEGSTKGQIQLVQSGP | |
| | ELKKPGETVKISCKASGYTFTDYSINWVKRAPGKGLKWMGWINT | |
| | ETREPAYAYDFRGRFAFSLETSASTAYLQINNLKYEDTATYFCAL | |
| | DYSYAMDYWGQGTSVTVSS | |
| BCMA VHH1 | QVKLEESGGGLVQAGRSLRLSCAASEHTFSSHVMGWFRQAPGKE | 68 |
| | RESVAVIGWRDISTSYADSVKGRFTISRDNAKKTLYLQMNSLKPE | |
| | DTAVYYCAARRIDAADFDSWGQGTQVTVSS | |
| BCMA VHH2 | EVQLVESGGGLVQAGGSLRLSCAASGRTFTMGWFRQAPGKEREF | 69 |
| | VAAISLSPTLAYYAESVKGRFTISRDNAKNTVVLQMNSLKPEDTA | |
| | LYYCAADRKSVMSIRPDYWGQGTQVTVSS | |
| BCMA antigen | MGSWSEFWVRLGAIRERLDALGGSEAELAAFEKEIAAFESELQA | 70 |
| binding domain | YKGKGNPEVEKLRYTAATIRRFLQAYRHN | |

Additional embodiments of the present disclosure provide full-length anti-BCMA CAR constructs comprising, consisting of, or consisting essentially of, any of the amino acid sequences of SEQ ID NOs: 71-92.

In some embodiments, the sequences for the full length anti-BCMA CAR constructs are selected from TABLE 2B.

TABLE 2B

| CAR | Amino Acid Sequence | SEQ ID NO: |
|---|---|---|
| HCVR1 | MALPVTALLLPLALLLHAARPQVQLVESGGGLVQPGGSLRLSCAASGF | 71 |
| CD828Z CAR | TFTNHAMSWVRQAPGKGLELVSSISGNGRTTYYADSVKGRFTISRDIS | |
| | KNTLDLQMNSLRAEDTAVYYCAKDGGETLVDSRGQGTLVTVSSFVPV | |
| | FLPAKPTTTPAPRPPTPAPTIASQPLSLRPEACRPAAGGAVHTRGLDFAC | |
| | DIYIWAPLAGTCGVLLLSLVITLYCNHRNRSKRSRLLHSDYMNMTPRR | |
| | PGPTRKHYQPYAPPRDFAAYRSRVKFSRSADAPAYQQGQNQLYNELN | |
| | LGRREEYDVLDKRRGRDPEMGGKPRRKNPQEGLYNELQKDKMAEAY | |
| | SEIGMKGERRRGKGHDGLYQGLSTATKDTYDALHMQALPPR | |
| HCVR2 | MALPVTALLLPLALLLHAARPQVQLVESGGGLVQPGGSLRLSCAASGF | 72 |
| CD828Z | TFSSHAMTWVRQAPGKGLEWVAAISGSGDFTHYADSVKGRFTISRDN | |
| CAR | SKNTVSLQMNNLRAEDTAVYYCAKDEDGGSLLGYRGQGTLVTVSSFV | |
| | PVFLPAKPTTTPAPRPPTPAPTIASQPLSLRPEACRPAAGGAVHTRGLDF | |
| | ACDIYIWAPLAGTCGVLLLSLVITLYCNHRNRSKRSRLLHSDYMNMTP | |
| | RRPGPTRKHYQPYAPPRDFAAYRSRVKFSRSADAPAYQQGQNQLYNE | |
| | LNLGRREEYDVLDKRRGRDPEMGGKPRRKNPQEGLYNELQKDKMAE | |
| | AYSEIGMKGERRRGKGHDGLYQGLSTATKDTYDALHMQALPPR | |
| HCVR3 | MALPVTALLLPLALLLHAARPEVQLLESGGGLVQPGGSLRLSCAASGF | 73 |
| CD828Z | TFSSYAMSWVRQAPGKGLEWVSSISGSGDYIYYADSVKGRFTISRDISK | |
| CAR | NTLYLQMNSLRAEDTAVYYCAKEGTGANSSLADYRGQGTLVTVSSFV | |
| | PVFLPAKPTTTPAPRPPTPAPTIASQPLSLRPEACRPAAGGAVHTRGLDF | |
| | ACDIYIWAPLAGTCGVLLLSLVITLYCNHRNRSKRSRLLHSDYMNMTP | |
| | RRPGPTRKHYQPYAPPRDFAAYRSRVKFSRSADAPAYQQGQNQLYNE | |
| | LNLGRREEYDVLDKRRGRDPEMGGKPRRKNPQEGLYNELQKDKMAE | |
| | AYSEIGMKGERRRGKGHDGLYQGLSTATKDTYDALHMQALPPR | |
| HCVR4 | MALPVTALLLPLALLLHAARPEVQLLESGGGLIQPGGSLRLSCAASGFT | 74 |
| CD828Z | FSSHAMTWVRQAPGKGLEWVSAISGSGDYTHYADSVKGRFTISRDNS | |
| CAR | KNTVYLQMNSLRAEDSAVYYCAKDEDGGSLLGHRGQGTLVTVSSFVP | |
| | VFLPAKPTTTPAPRPPTPAPTIASQPLSLRPEACRPAAGGAVHTRGLDFA | |
| | CDIYIWAPLAGTCGVLLLSLVITLYCNHRNRSKRSRLLHSDYMNMTPR | |
| | RPGPTRKHYQPYAPPRDFAAYRSRVKFSRSADAPAYQQGQNQLYNEL | |
| | NLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| HCVR1 | MALPVTALLLPLALLLHAARPQVQLVESGGGLVQPGGSLRLSCAASGF | 75 |
| CD8BBZ | TFTNHAMSWVRQAPGKGLELVSSISGNGRTTYYADSVKGRFTISRDIS | |
| CAR | KNTLDLQMNSLRAEDTAVYYCAKDGGETLVDSRGQGTLVTVSSFVPV | |
| | FLPAKPTTTPAPRPPTPAPTIASQPLSLRPEACRPAAGGAVHTRGLDFAC | |
| | DIYIWAPLAGTCGVLLLSLVITLYCNHRNKRGRKKLLYIFKQPFMRPVQ | |
| | TTQEEDGCSCRFPEEEEGGCELRVKFSRSADAPAYQQGQNQLYNELNL | |
| | GRREEYDVLDKRRGRDPEMGGKPRRKNPQEGLYNELQKDKMAEAYS | |
| | EIGMKGERRRGKGHDGLYQGLSTATKDTYDALHMQALPPR | |
| HCVR2 | MALPVTALLLPLALLLHAARPQVQLVESGGGLVQPGGSLRLSCAASGF | 76 |
| CD8BBZ | TFSSHAMTWVRQAPGKGLEWVAAISGSGDFTHYADSVKGRFTISRDN | |
| CAR | SKNTVSLQMNNLRAEDTAVYYCAKDEDGGSLLGYRGQGTLVTVSSFV | |
| | PVFLPAKPTTTPAPRPPTPAPTIASQPLSLRPEACRPAAGGAVHTRGLDF | |
| | ACDIYIWAPLAGTCGVLLLSLVITLYCNHRNKRGRKKLLYIFKQPFMRP | |
| | VQTTQEEDGCCRFPEEEEGGCELRVKFSRSADAPAYQQGQNQLYNEL | |
| | NLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| HCVR3 | MALPVTALLLPLALLLHAARPEVQLLESGGGLVQPGGSLRLSCAASGF | 77 |
| CD8BBZ | TFSSYAMSWVRQAPGKGLEWVSSISGSGDYIYYADSVKGRFTISRDISK | |
| CAR | NTLYLQMNSLRAEDTAVYYCAKEGTGANSSLADYRGQGTLVTVSSFV | |
| | PVFLPAKPTTTPAPRPPTPAPTIASQPLSLRPEACRPAAGGAVHTRGLDF | |
| | ACDIYIWAPLAGTCGVLLLSLVITLYCNHRNKRGRKKLLYIFKQPFMRP | |
| | VQTTQEEDGCSCRFPEEEEGGCELRVKFSRSADAPAYQQGQNQLYNEL | |
| | NLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| HCVR4 | MALPVTALLLPLALLLHAARPEVQLLESGGGLIQPGGSLRLSCAASGFT | 78 |
| CD8BBZ | FSSHAMTWVRQAPGKGLEWVSAISGSGDYTHYADSVKGRFTISRDNS | |
| CAR | KNTVYLQMNSLRAEDSAVYYCAKDEDGGSLLGHRGQGTLVTVSSFVP | |
| | VFLPAKPTTTPAPRPPTPAPTIASQPLSLRPEACRPAAGGAVHTRGLDFA | |
| | CDIYIWAPLAGTCGVLLLSLVITLYCNHRNKRGRKKLLYIFKQPFMRPV | |
| | QTTQEEDGCSCRFPEEEEGGCELRVKFSRSADAPAYQQGQNQLYNELN | |
| | LGRREEYDVLDKRRGRDPEMGGKPRRKNPQEGLYNELQKDKMAEAY | |
| | SEIGMKGERRRGKGHDGLYQGLSTATKDTYDALHMQALPPR | |
| HCVR1 | MALPVTALLLPLALLLHAARPQVQLVESGGGLVQPGGSLRLSCAASGF | 79 |
| CD8ICOSZ | TFTNHAMSWVRQAPGKGLELVSSISGNGRTTYYADSVKGRFTISRDIS | |
| CAR | KNTLDLQMNSLRAEDTAVYYCAKDGGETLVDSRGQGTLVTVSSFVPV | |
| | FLPAKPTTTPAPRPPTPAPTIASQPLSLRPEACRPAAGGAVHTRGLDFAC | |
| | DIYIWAPLAGTCGVLLLSLVITLYCNHRNCWLTKKKYSSSVHDPNGEY | |
| | MFMRAVNTAKKSRLTDVTLRVKFSRSADAPAYQQGQNQLYNELNLG | |
| | RREEYDVLDKRRGRDPEMGGKPRRKNPQEGLYNELQKDKMAEAYSEI | |
| | GMKGERRRGKGHDGLYQGLSTATKDTYDALHMQALPPR | |
| HCVR2 | MALPVTALLLPLALLLHAARPQVQLVESGGGLVQPGGSLRLSCAASGF | 80 |
| CD8ICOSZ | TFSSHAMTWVRQAPGKGLEWVAAISGSGDFTHYADSVKGRFTISRDN | |
| CAR | SKNTVSLQMNNLRAEDTAVYYCAKDEDGGSLLGYRGQGTLVTVSSFV | |
| | PVFLPAKPTTTPAPRPPTPAPTIASQPLSLRPEACRPAAGGAVHTRGLDF | |
| | ACDIYIWAPLAGTCGVLLLSLVITLYCNHRNCWLTKKKYSSSVHDPNG | |
| | EYMFMRAVNTAKKSRLTDVTLRVKFSRSADAPAYQQGQNQLYNELN | |
| | LGRREEYDVLDKRRGRDPEMGGKPRRKNPQEGLYNELQKDKMAEAY | |
| | SEIGMKGERRRGKGHDGLYQGLSTATKDTYDALHMQALPPR | |
| HCVR3 | MALPVTALLLPLALLLHAARPEVQLLESGGGLVQPGGSLRLSCAASGF | 81 |
| CD8ICOSZ | TFSSYAMSWVRQAPGKGLEWVSSISGSGDYIYYADSVKGRFTISRDISK | |
| CAR | NTLYLQMNSLRAEDTAVYYCAKEGTGANSSLADYRGQGTLVTVSSFV | |
| | PVFLPAKPTTTPAPRPPTPAPTIASQPLSLRPEACRPAAGGAVHTRGLDF | |
| | ACDIYIWAPLAGTCGVLLLSLVITLYCNHRNCWLTKKKYSSSVHDPNG | |
| | EYMFMRAVNTAKKSRLTDVTLRVKFSRSADAPAYQQGQNQLYNELN | |
| | LGRREEYDVLDKRRGRDPEMGGKPRRKNPQEGLYNELQKDKMAEAY | |
| | SEIGMKGERRRGKGHDGLYQGLSTATKDTYDALHMQALPPR | |
| HCVR4 | MALPVTALLLPLALLLHAARPEVQLLESGGGLIQPGGSLRLSCAASGFT | 82 |
| CD8ICOSZ | FSSHAMTWVRQAPGKGLEWVSAISGSGDYTHYADSVKGRFTISRDNS | |
| CAR | KNTVYLQMNSLRAEDSAVYYCAKDEDGGSLLGHRGQGTLVTVSSFVP | |
| | VFLPAKPTTTPAPRPPTPAPTIASQPLSLRPEACRPAAGGAVHTRGLDFA | |
| | CDIYIWAPLAGTCGVLLLSLVITLYCNHRNCWLTKKKYSSSVHDPNGE | |
| | YMFMRAVNTAKKSRLTDVTLRVKFSRSADAPAYQQGQNQLYNELNL | |
| | GRREEYDVLDKRRGRDPEMGGKPRRKNPQEGLYNELQKDKMAEAYS | |
| | EIGMKGERRRGKGHDGLYQGLSTATKDTYDALHMQALPPR | |
| scFv | MALPVTALLLPLALLLHAARPDIVLTQSPPSLAMSLGKRATISCRASES | 83 |
| BCMA1 | VTILGSHLIYWYQQKPGQPPTLLIQLASNVQTGVPARFSGSGSRTDFTL | |
| CAR | TIDPVEEDDVAVYYCLQSRTIPRTFGGGTKLEIKGSTSGSGKPGSGEGST | |
| | KGQIQLVQSGPELKKPGETVKISCKASGYTFRHYSMNWVKQAPGKGL | |
| | KWMGRINTESGVPIYADDFKGRFAFSVETSASTAYLVINNLKDEDTAS | |
| | YFCSNDYLYSLDFWGQGTALTVSSFVPVFLPAKPTTTPAPRPPTPAPTIA | |
| | SQPLSLRPEACRPAAGGAVHTRGLDFACDIYIWAPLAGTCGVLLLSLVI | |
| | TLYCNHRNRSKRSRLLHSDYMNMTPRRPGPTRKHYQPYAPPRDFAAY | |
| | RSRVKFSRSADAPAYQQGQNQLYNELNLGRREEYDVLDKRRGRDPEM | |
| | GGKPRRKNPQEGLYNELQKDKMAEAYSEIGMKGERRRGKGHDGLYQ | |
| | GLSTATKDTYDALHMQALPPR | |
| scFv | MALPVTALLLPLALLLHAARPDIVLTQSPPSLAMSLGKRATISCRASES | 84 |
| BCMA2 | VTILGSHLIHWYQQKPGQPPTLLIQLASNVQTGVPARFSGSGSRTDFTL | |
| CAR | TIDPVEEDDVAVYYCLQSRTIPRTFGGGTKLEIKGSTSGSGKPGSGEGST | |
| | KGQIQLVQSGPELKKPGETVKISCKASGYTFTDYSINWVKRAPGKGLK | |
| | WMGWINTETREPAYAYDFRGRFAFSLETSASTAYLQINNLKYEDTATY | |
| | FCALDYSYAMDYWGQGTSVTVSSFVPVFLPAKPTTTPAPRPPTPAPTIA | |
| | SQPLSLRPEACRPAAGGAVHTRGLDFACDIYIWAPLAGTCGVLLLSLVI | |
| | TLYCNHRNRSKRSRLLHSDYMNMTPRRPGPTRKHYQPYAPPRDFAAY | |
| | RSRVKFSRSADAPAYQQGQNQLYNELNLGRREEYDVLDKRRGRDPEM | |
| | GGKPRRKNPQEGLYNELQKDKMAEAYSEIGMKGERRRGKGHDGLYQ | |
| | GLSTATKDTYDALHMQALPPR | |
| scFv | MALPVTALLLPLALLLHAARPDIVLTQSPPSLAMSLGKRATISCRASES | 85 |
| BCMA3 | VTILGSHLIYWYQQKPGQPPTLLIQLASNVQTGVPARFSGSGSRTDFTL | |
| CAR | TIDPVEEDDVAVYYCLQSRTIPRTFGGGTKLEIKGSTSGSGKPGSGEGST | |
| | KGQIQLVQSGPELKKPGETVKISCKASGYTFTHYSMNWVKQAPGKGL | |
| | KWMGRINTETGEPLYADDFKGRFAFSLETSASTAYLVINNLKNEDTAT | |
| | FFCSNDYLYSCDYWGQGTTLTVSSFVPVFLPAKPTTTPAPRPPTPAPTIA | |
| | SQPLSLRPEACRPAAGGAVHTRGLDFACDIYIWAPLAGTCGVLLLSLVI | |
| | TLYCNHRNRSKRSRLLHSDYMNMTPRRPGPTRKHYQPYAPPRDFAAY | |
| | RSRVKFSRSADAPAYQQGQNQLYNELNLGRREEYDVLDKRRGRDPEM | |
| | GGKPRRKNPQEGLYNELQKDKMAEAYSEIGMKGERRRGKGHDGLYQ | |
| | GLSTATKDTYDALHMQALPPR | |
| scFv | MLLLVTSLLLCELPHPAFLLIPDIVLTQSPPSLAMSLGKRATISCRASESV | 86 |
| BCMA4 | TILGSHLIHWYQQKPGQPPTLLIQLASNVQTGVPARFSGSGSRTDFTLTI | |
| CAR | DPVEEDDVAVYYCLQSRTIPRTFGGGTKLEIKGSTSGSGKPGSGEGSTK | |
| | GQIQLVQSGPELKKPGETVKISCKASGYTFTDYSINWVKRAPGKGLKW | |
| | MGWINTETREPAYAYDFRGRFAFSLETSASTAYLQINNLKYEDTATYF | |
| | CALDYSYAMDYWGQGTSVTVSSAAAFVPVFLPAKPTTTPAPRPPTPAP | |
| | TIASQPLSLRPEACRPAAGGAVHTRGLDFACDIYIWAPLAGTCGVLLLS | |
| | LVITLYCNHRNRSKRSRLLHSDYMNMTPRRPGPTRKHYQPYAPPRDFA | |
| | AYRSRVKFSRSADAPAYQQGQNQLYNELNLGRREEYDVLDKRRGRDP | |
| | EMGGKPRRKNPQEGLYNELQKDKMAEAYSEIGMKGERRRGKGHDGL | |
| | YQGLSTATKDTYDALHMQALPPR | |
| scFv | MALPVTALLLPLALLLHAARPDIVLTQSPPSLAMSLGKRATISCRASES | 87 |
| BCMA5 | VTILGSHLIHWYQQKPGQPPTLLIQLASNVQTGVPARFSGSGSRTDFTL | |
| CAR | TIDPVEEDDVAVYYCLQSRTIPRTFGGGTKLEIKGSTSGSGKPGSGEGST | |
| | KGQIQLVQSGPELKKPGETVKISCKASGYTFTDYSINWVKRAPGKGLK | |
| | WMGWINTETREPAYAYDFRGRFAFSLETSASTAYLQINNLKYEDTATY | |
| | FCALDYSYAMDYWGQGTSVTVSSAAAFVPVFLPAKPTTTPAPRPPTPA | |
| | PTIASQPLSLRPEACRPAAGGAVHTRGLDFACDIYIWAPLAGTCGVLLL | |
| | SLVITLYCNHRNRSKRSRLLHSDYMNMTPRRPGPTRKHYQPYAPPRDF | |
| | AAYRSRVKFSRSADAPAYQQGQNQLYNELNLGRREEYDVLDKRRGR | |
| | DPEMGGKPRRKNPQEGLYNELQKDKMAEAYSEIGMKGERRRGKGHD | |
| | GLYQGLSTATKDTYDALHMQALPPR | |
| scFv | MALPVTALLLPLALLLHAARPDIVLTQSPPSLAMSLGKRATISCRASES | 88 |
| BCMA2-BB | VTILGSHLIHWYQQKPGQPPTLLIQLASNVQTGVPARFSGSGSRTDFTL | |
| CAR | TIDPVEEDDVAVYYCLQSRTIPRTFGGGTKLEIKGSTSGSGKPGSGEGST | |
| | KGQIQLVQSGPELKKPGETVKISCKASGYTFTDYSINWVKRAPGKGLK | |
| | WMGWINTETREPAYAYDFRGRFAFSLETSASTAYLQINNLKYEDTATY | |
| | FCALDYSYAMDYWGQGTSVTVSSAAAFVPVFLPAKPTTTPAPRPPTPA | |
| | PTIASQPLSLRPEACRPAAGGAVHTRGLDFACDIYIWAPLAGTCGVLLL | |
| | SLVITLYCNHRNRFSVVKRGRKKLLYIFKQPFMRPVQTTQEEDGCSCRF | |
| | PEEEEGGCELRVKFSRSADAPAYQQGQNQLYNELNLGRREEYDVLDK | |
| | RRGRDPEMGGKPRRKNPQEGLYNELQKDKMAEAYSEIGMKGERRRG | |
| | KGHDGLYQGLSTATKDTYDALHMQALPPR | |
| scFv | MALPVTALLLPLALLLHAARPDIVLTQSPPSLAMSLGKRATISCRASES | 89 |
| BCMA2 | VTILGSHLIHWYQQKPGQPPTLLIQLASNVQTGVPARFSGSGSRTDFTL | |
| OX40 CAR | TIDPVEEDDVAVYYCLQSRTIPRTFGGGTKLEIKGSTSGSGKPGSGEGST | |
| | KGQIQLVQSGPELKKPGETVKISCKASGYTFTDYSINWVKRAPGKGLK | |
| | WMGWINTETREPAYAYDFRGRFAFSLETSASTAYLQINNLKYEDTATY | |
| | FCALDYSYAMDYWGQGTSVTVSSAAAFVPVFLPAKPTTTPAPRPPTPA | |
| | PTIASQPLSLRPEACRPAAGGAVHTRGLDFACDIYIWAPLAGTCGVLLL | |
| | SLVITLYCNHRNRRDQRLPPDAHKPPGGGSFRTPIQEEQADAHSTLAKI | |
| | RVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQG | |
| | LSTATKDTYDALHMQALPPR | |
| scFv | MALPVTALLLPLALLLHAARPDIVLTQSPPSLAMSLGKRATISCRASES | 90 |
| BCMA2 | VTILGSHLIHWYQQKPGQPPTLLIQLASNVQTGVPARFSGSGSRTDFTL | |
| BBOX40 | TIDPVEEDDVAVYYCLQSRTIPRTFGGGTKLEIKGSTSGSGKPGSGEGST | |
| CAR | KGQIQLVQSGPELKKPGETVKISCKASGYTFTDYSINWVKRAPGKGLK | |
| | WMGWINTETREPAYAYDFRGRFAFSLETSASTAYLQINNLKYEDTATY | |
| | FCALDYSYAMDYWGQGTSVTVSSAAAFVPVFLPAKPTTTPAPRPPTPA | |
| | PTIASQPLSLRPEACRPAAGGAVHTRGLDFACDIYIWAPLAGTCGVLLL | |
| | SLVITLYCNHRNRFSVVKRGRKKLLYIFKQPFMRPVQTTQEEDGCCRF | |
| | PEEEEGGCELRRDQRLPPDAHKPPGGGSFRTPIQEEQADAHSTLAKIRV | |
| | KFSRSADAPAYQQGQNQLYNELNLGRREEYDVLDKRRGRDPEMGGK | |
| | PRRKNPQEGLYNELQKDKMAEAYSEIGMKGERRRGKGHDGLYQGLS | |
| | TATKDTYDALHMQALPPR | |
| BCMA | MALPVTALLLPLALLLHAARPQVKLEESGGGLVQAGRSLRLSCAASEH | 91 |
| VHH | TFSSHVMGWFRQAPGKERESVAVIGWRDISTSYADSVKGRFTISRDNA | |
| CAR | KKTLYLQMNSLKPEDTAVYYCAARRIDAADFDSWGQGTQVTVSSGGG | |
| | GSEVOLVESGGGLVQAGGSLRLSCAASGRTFTMGWFRQAPGKEREFV | |
| | AAISLSPTLAYYAESVKGRFTISRDNAKNTVVLQMNSLKPEDTALYYC | |
| | AADRKSVMSIRPDYWGQGTQVTVSSTSTTTPAPRPPTPAPTIASQPLSLR | |
| | PEACRPAAGGAVHTRGLDFACDIYIWAPLAGTCGVLLLSLVITLYCKR | |
| | GRKKLLYIFKQPFMRPVQTTQEEDGCSCRFPEEEEGGCELRVKFSRSAD | |
| | APAYQQGQNQLYNELNLGRREEYDVLDKRRGRDPEMGGKPRRKNPQ | |
| | EGLYNELQKDKMAEAYSEIGMKGERRRGKGHDGLYQGLSTATKDTY | |
| | DALHMQALPPR | |
| BCMA | MAFLWLLSCWALLGTTFGDYKDDDDKGGGGSGGGGSMGSWSEFWV | 92 |
| CAR | RLGAIRERLDALGGSEAELAAFEKEIAAFESELQAYKGKGNPEVEKLRY | |
| | TAATIRRFLQAYRHNGGGGSGGGGSGTTTPAPRPPTPAPTIASQPLSLRP | |
| | EACRPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRSADA | |
| | PAYKQGQNQLYNELNLGRREEYDVLDKRRGRDPEMGGKPRRKNPQE | |
| | GLYNELQKDKMAEAYSEIGMKGERRRGKGHDGLYQGLSTATKDTYD | |
| | ALHMQALPPR | |

In some embodiments, an anti-BCMA CAR construct of the present disclosure may comprise one or more components comprising any of the amino acid sequences of SEQ ID NOs: 93-104.

In some embodiments, the sequences of components of anti-BCMA CAR constructs of the present disclosure are selected from TABLE 2C.

TABLE 2C

| | | SEQ |
|---|---|---|
| | | ID |
| Component | Amino Acid Sequence | NO: |
| scFv linker | GSTSGSGKPGSGEGSTKG | 93 |
| Linker | GGGGS | 94 |
| CD8-alpha | MALPVTALLLPLALLLHAARP | 95 |
| signal | | |
| peptide | | |
| CD8-alpha | FVPVFLPAKPTTTPAPRPPTPAPT | 96 |
| | IASQPLSLRPEACRPAAGGAVHTR | |
| hinge 1 | GLDFACD | |
| CD8-alpha | TTTPAPRPPTPAPTIASQPLSLRP | 97 |
| hinge 2 | EACRPAAGGAVHTRGLDFACD | |
| CD8-alpha | IYIWAPLAGTCGVLLLSLVITLYCN | 98 |
| TM 1 | | |
| CD8-alpha | IYIWAPLAGTCGVLLLSLVITLYC | 99 |
| TM 2 | | |
| 4-1BB | RFSVVKRGRKKLLYIFKQPFMRPVQ | 100 |
| ICD 1 | TTQEEDGCSCRFPEEEEGGCEL | |
| 4-1BB | KRGRKKLLYIFKQPFMRPVQTTQEE | 101 |
| ICD 2 | DGCSCRFPEEEEGGCEL | |
| CD28 ICD | RSKRSRLLHSDYMNMTPRRPGPTRK | 102 |
| | HYQPYAPPRDFAAYRS | |
| OX40 ICD | RRDQRLPPDAHKPPGGGSFRTPIQE | 103 |
| | EQADAHSTLAKI | |
| CD3-zeta | RVKFSRSADAPAYQQGQNQLYNELN | 104 |
| ICD | LGRREEYDVLDKRRGRDPEMGGKPR | |
| | RKNPQEGLYNELQKDKMAEAYSEIG | |
| | MKGERRRGKGHDGLYQGLSTATKD | |
| | TYDALHMQALPPR | |

In some embodiments, a CAR of the present disclosure may be idecabtagene vicleucel or ciltacabtagene autoleucel.

Included in the scope of the present disclosure are functional portions of the inventive CARs described herein. The term “functional portion” when used in reference to a CAR refers to any part or fragment of the CAR of the present disclosure, which part or fragment retains the biological activity of the CAR of which it is a part (the parent CAR). Functional portions encompass, for example, those parts of a CAR that retain the ability to recognize target cells, or detect, treat, or prevent cancer, to a similar extent, the same extent, or to a higher extent, as the parent CAR. In reference to the parent CAR, the functional portion can comprise, for instance, about 10%, 25%, 30%, 50%, 68%, 80%, 90%, 95%, or more, of the parent CAR.

The functional portion can comprise additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent CAR.

Included in the scope of the present disclosure are functional variants of the inventive CARs described herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, about 50%, about 75%, about 80%, about 90%, about 91%, about 92%, about 93%, about 94%, about 95%, about 96%, about 97%, about 98%, about 99% or more identical in amino acid sequence to the parent CAR.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the inventive CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The CAR can consist essentially of the specified amino acid sequence or sequences described herein, such that other components, e.g., other amino acids, do not materially change the biological activity of the functional variant.

The CARs of embodiments of the present disclosure (including functional portions and functional variants) can be of any length, i.e., can comprise any number of amino acids, provided that the CARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CARs of embodiments of the present disclosure (including functional portions and functional variants of the present disclosure) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, a-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,β-diaminopropionic acid, homophenylalanine, and α-tert-butylglycine.

The CARs of embodiments of the present disclosure (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CARs of embodiments of the present disclosure (including functional portions and functional variants thereof) can be obtained by methods known in the art. The CARs may be made by any suitable method of making polypeptides or proteins, including de nova synthesis. Also, the CARs can be recombinantly produced using the nucleic acids described herein using standard recombinant methods. See, for instance, Green et al., Molecular Cloning: A Laboratory Manual, 4th ed., Cold Spring Harbor Press, Cold Spring Harbor, NY 2012. Further, portions of some of the CARs of the present disclosure (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies, such as Synpep (Dublin, CA), Peptide Technologies Corp. (Gaithersburg, MD), and Multiple Peptide Systems (San Diego, CA). In this respect, the inventive CARs can be synthetic, recombinant, isolated, and/or purified.

Further provided by an embodiment of the present disclosure is a nucleic acid comprising a nucleotide sequence encoding any of the CARs described herein (including functional portions and functional variants thereof). The nucleic acids of the present disclosure may comprise a nucleotide sequence encoding any of the leader sequences, antigen binding domains, transmembrane domains, linkers, and/or intracellular T cell signaling domains described herein.

In an embodiment, the nucleic acid comprises a nucleotide sequence that encodes a leader sequence, anti-CD22 and anti-CD19 antigen binding domains (including a light chain variable region and a heavy chain variable region joined by linkers), a CD8 hinge domain, a CD8 transmembrane domain, a 4-1BB intracellular T cell signaling domain, and a CD3ζ intracellular T cell signaling domain. For example, the nucleic acid may comprise a nucleotide sequence that encodes anti-CD22 and anti-CD19 antigen binding domains (including a light chain variable region and a heavy chain variable region joined by linkers), a CD8 hinge domain (SEQ ID NO: 34), a CD8 transmembrane domain (SEQ ID NO: 35), a 4-1BB intracellular T cell signaling domain (SEQ ID NO: 36), and a CD3ζ intracellular T cell signaling domain (SEQ ID NO: 37). In an embodiment of the present disclosure, the nucleic acid comprises a nucleotide sequence that encodes a CD8 hinge domain, a CD8 transmembrane domain, a 4-1BB intracellular T cell signaling domain, and a CD32 intracellular T cell signaling domain comprising the nucleotide sequence of SEQ ID NO: 38. In an embodiment of the present disclosure, the nucleic acid may comprise, consist of, or consist essentially of, the nucleotide sequence of SEQ ID NO: 31 or 32, which encodes a full-length CAR comprising the amino acid sequence of SEQ ID NO: 29 or 30, respectively. The nucleotide sequences for SEQ ID NOs: 31, 32, 34-38 are as in TABLE 3.

TABLE 3

| SEQ ID NO | Nucleic Acid Sequence |
|---|---|
| 31 | GCCACCATGCTGCTGCTCGTGACAAGCCTGCTGCTGTGCG |
| | AGCTGCCCCACCCTGCCTTTCTGCTGATCCCCGACATCCA |
| | GATGACCCAGACCACCAGCAGCCTGAGCGCCAGCCTGGGC |
| | GATAGAGTGACCATCAGCTGCAGAGCCAGCCAGGACATCA |
| | GCAAGTACCTGAACTGGTATCAGCAGAAACCCGACGGCAC |
| | CGTGAAGCTGCTGATCTACCACACCAGCAGACTGCACAGC |
| | GGCGTGCCCAGCAGATTTTCTGGCAGCGGCTCCGGCACCG |
| | ACTACAGCCTGACCATCTCCAACCTGGAACAGGAAGATAT |
| | CGCTACCTACTTCTGTCAGCAAGGCAACACCCTGCCCTAC |
| | ACCTTCGGCGGAGGCACCAAGCTGGAAATCACAGGCGGCG |
| | GAGGATCCCAGGTGCAGCTGCAGCAGTCTGGACCCGGCCT |
| | CGTGAAGCCTAGCCAGACCCTGTCTCTGACCTGCGCCATC |
| | AGCGGCGATAGCGTGTCCAGCAATAGCGCCGCCTGGAACT |
| | GGATCCGGCAGAGCCCTTCTAGAGGCCTGGAATGGCTGGG |
| | CCGGACCTACTACCGGTCCAAGTGGTACAACGACTACGCC |
| | GTGTCCGTGAAGTCCCGGATCACCATCAACCCCGACACCA |
| | GCAAGAACCAGTTCTCCCTGCAGCTGAACAGCGTGACCCC |
| | CGAGGATACCGCCGTGTACTACTGCGCCAGAGAAGTGACC |
| | GGCGACCTGGAAGATGCCTTCGACATCTGGGGCCAGGGCA |
| | CAATGGTCACCGTGTCTAGCGGCAGCACAAGCGGCTCTGG |
| | CAAGCCTGGATCTGGCGAGGGCTCTACCAAGGGCGATATT |
| | CAGATGACACAGAGCCCCTCCAGCCTGTCCGCCTCTGTGG |
| | GAGACAGAGTGACAATCACCTGTCGGGCCTCCCAGACCAT |
| | CTGGTCCTATCTGAATTGGTATCAGCAGCGGCCTGGCAAG |
| | GCCCCCAACCTGCTGATCTATGCCGCCAGCTCTCTGCAGT |
| | CCGGCGTGCCATCTAGATTCAGCGGCAGAGGCAGCGGCAC |
| | CGATTTCACCCTGACAATTAGCAGTCTGCAGGCCGAGGAC |
| | TTCGCCACCTACTATTGCCAGCAGAGCTACAGCATCCCCC |
| | AGACCTTCGGCCAGGGAACAAAACTGGAAATCAAAGGGGG |
| | AGGCGGCAGCGAAGTGAAACTGCAGGAATCTGGCCCTGGC |
| | CTGGTGGCCCCAAGCCAGTCTCTGAGCGTGACCTGTACCG |
| | TGTCTGGCGTGTCCCTGCCCGATTACGGCGTGTCCTGGAT |
| | CAGACAGCCCCCCAGAAAGGGACTGGAATGGCTGGGAGTG |
| | ATCTGGGGCAGCGAGACAACCTACTACAACAGCGCCCTGA |
| | AGTCCAGGCTGACCATCATCAAGGACAACTCCAAGAGCCA |
| | GGTGTTCCTGAAGATGAATTCCCTGCAGACCGACGACACC |
| | GCCATCTATTACTGTGCCAAGCACTACTACTACGGCGGCA |
| | GCTACGCCATGGACTACTGGGGACAGGGAACCTCCGTGAC |
| | CGTGTCCTCTTCCGGAACCACGACGCCAGCGCCGCGACCA |
| | CCAACACCGGCGCCCACCATCGCGTCGCAGCCCCTGTCCC |
| | TGCGCCCAGAGGCGTGCCGGCCAGCGGCGGGGGGCGCAGT |
| | GCACACGAGGGGGCTGGACTTCGCCTGTGATATCTACATC |
| | TGGGCGCCCTTGGCCGGGACTTGTGGGGTCCTTCTCCTGT |
| | CACTGGTTATCACCCTTTACTGCAAACGGGGCAGAAAGAA |
| | ACTCCTGTATATATTCAAACAACCATTTATGAGACCAGTA |
| | CAAACTACTCAAGAGGAAGATGGCTGTAGCTGCCGATTTC |
| | CAGAAGAAGAAGAAGGAGGATGTGAACTGAGAGTGAAGTT |
| | CAGCAGGAGCGCAGACGCCCCCGCGTACAAGCAGGGCCAG |
| | AACCAGCTCTATAACGAGCTCAATCTAGGACGAAGAGAGG |
| | AGTACGATGTTTTGGACAAGAGACGTGGCCGGGACCCTGA |
| | GATGGGGGGAAAGCCGAGAAGGAAGAACCCTCAGGAAGGC |
| | CTGTACAATGAACTGCAGAAAGATAAGATGGCGGAGGCCT |
| | ACAGTGAGATTGGGATGAAAGGCGAGCGCCGGAGGGGCAA |
| | GGGGCACGATGGCCTTTACCAGGGTCTCAGTACAGCCACC |
| | AAGGACACCTACGACGCCCTTCACATGCAGGCCCTGCCCC |
| | CTCGCTAA |
| 32 | GCCACCATGCTGCTGCTCGTGACATCTCTGCTGCTGTGCG |
| | AGCTGCCCCACCCCGCCTTTCTGCTGATTCCTCAGGTGCA |
| | GCTGCAGCAGTCTGGCCCTGGCCTCGTGAAGCCTAGCCAG |
| | ACCCTGAGCCTGACCTGTGCCATCAGCGGCGATAGCGTGT |
| | CCAGCAATAGCGCCGCCTGGAACTGGATCAGACAGAGCCC |
| | TAGCAGAGGCCTGGAATGGCTGGGCCGGACCTACTACCGG |
| | TCCAAGTGGTACAACGACTACGCCGTGTCCGTGAAGTCCC |
| | GGATCACCATCAACCCCGACACCAGCAAGAACCAGTTCTC |
| | CCTGCAGCTGAACAGCGTGACCCCCGAGGATACCGCCGTG |
| | TACTACTGCGCCAGAGAAGTGACCGGCGACCTGGAAGATG |
| | CCTTCGACATCTGGGGCCAGGGCACAATGGTCACCGTGTC |
| | TAGCGGAGGCGGCGGAAGCGACATCCAGATGACACAGAGC |
| | CCCAGCTCCCTGAGCGCCAGCGTGGGAGACAGAGTGACCA |
| | TCACCTGTCGGGCCAGCCAGACCATCTGGTCCTACCTGAA |
| | CTGGTATCAGCAGCGGCCTGGCAAGGCCCCCAACCTGCTG |
| | ATCTATGCCGCCAGCTCACTGCAGAGCGGCGTGCCCAGCA |
| | GATTTTCCGGCAGAGGCAGCGGCACCGACTTCACCCTGAC |
| | AATCAGTTCCCTGCAGGCCGAGGACTTCGCCACCTACTAC |
| | TGCCAGCAGAGCTACAGCATCCCCCAGACCTTCGGCCAGG |
| | GGACCAAGCTGGAAATCAAGGGCGGAGGGGGATCTGGCGG |
| | CGGAGGATCTGGGGGAGGCGGCAGTGGGGGCGGAGGAAGT |
| | GGCGGGGGAGGCTCTGATATTCAGATGACCCAGACCACCT |
| | CCAGCCTGTCCGCCAGCCTGGGCGATCGCGTGACCATCTC |
| | TTGCAGAGCCAGCCAGGACATCAGCAAGTATCTGAATTGG |
| | TATCAGCAGAAACCCGACGGCACCGTGAAGCTGCTGATCT |
| | ACCACACCAGCAGACTGCACTCCGGCGTGCCATCCAGATT |
| | CAGCGGCTCTGGCTCCGGCACCGATTATAGCCTGACCATC |
| | AGCAACCTGGAACAGGAAGATATCGCTACCTACTTTTGTC |
| | AGCAAGGCAACACCCTGCCCTACACCTTCGGCGGAGGCAC |
| | AAAACTGGAAATTACCGGCAGCACCAGCGGCAGCGGAAAG |
| | CCTGGAAGCGGCGAGGGAAGCACCAAGGGCGAAGTGAAAC |
| | TGCAGGAAAGCGGACCCGGACTGGTGGCCCCAAGCCAGTC |
| | TCTGAGCGTGACATGTACCGTGTCCGGCGTGTCCCTGCCC |
| | GACTATGGCGTGTCCTGGATCAGGCAGCCCCCCAGAAAGG |
| | GACTGGAATGGCTGGGAGTGATCTGGGGCAGCGAGACAAC |
| | CTACTACAACAGCGCCCTGAAGTCCAGGCTGACCATTATC |
| | AAGGACAACTCCAAGAGCCAGGTGTTCCTGAAGATGAACA |
| | GCCTGCAGACCGACGACACAGCCATCTACTATTGCGCCAA |
| | GCACTACTACTACGGCGGCAGCTACGCCATGGACTACTGG |
| | GGACAGGGAACCTCCGTGACCGTGTCCTCTACCACGACGC |
| | CAGCGCCGCGACCACCAACACCGGCGCCCACCATCGCGTC |
| | GCAGCCCCTGTCCCTGCGCCCAGAGGCGTGCCGGCCAGCG |
| | GCGGGGGGCGCAGTGCACACGAGGGGGCTGGACTTCGCCT |
| | GTGATATCTACATCTGGGCGCCCTTGGCCGGGACTTGTGG |
| | GGTCCTTCTCCTGTCACTGGTTATCACCCTTTACTGCAAA |
| | CGGGGCAGAAAGAAACTCCTGTATATATTCAAACAACCAT |
| | TTATGAGACCAGTACAAACTACTCAAGAGGAAGATGGCTG |
| | TAGCTGCCGATTTCCAGAAGAAGAAGAAGGAGGATGTGAA |
| | CTGAGAGTGAAGTTCAGCAGGAGCGCAGACGCCCCCGCGT |
| | ACAAGCAGGGCCAGAACCAGCTCTATAACGAGCTCAATCT |
| | AGGACGAAGAGAGGAGTACGATGTTTTGGACAAGAGACGT |
| | GGCCGGGACCCTGAGATGGGGGGAAAGCCGAGAAGGAAGA |
| | ACCCTCAGGAAGGCCTGTACAATGAACTGCAGAAAGATAA |
| | GATGGCGGAGGCCTACAGTGAGATTGGGATGAAAGGCGAG |
| | CGCCGGAGGGGCAAGGGGCACGATGGCCTTTACCAGGGTC |
| | TCAGTACAGCCACCAAGGACACCTACGACGCCCTTCACAT |
| | GCAGGCCCTGCCCCCTCGCTAA |
| 34 | ACCACGACGCCAGCGCCGCGACCACCAACACCGGCGCCCA |
| | CCATCGCGTCGCAGCCCCTGTCCCTGCGCCCAGAGGCGTG |
| | CCGGCCAGCGGCGGGGGGCGCAGTGCACACGAGGGGGCTG |
| | GACTTCGCCTGTGAT |
| 35 | ATCTACATCTGGGCGCCCTTGGCCGGGACTTGTGGGGTCC |
| | TTCTCCTGTCACTGGTTATCACCCTTTACTGC |
| 36 | AAACGGGGCAGAAAGAAACTCCTGTATATATTCAAACAAC |
| | CATTTATGAGACCAGTACAAACTACTCAAGAGGAAGATGG |
| | CTGTAGCTGCCGATTTCCAGAAGAAGAAGAAGGAGGATGT |
| | GAACTG |
| 37 | AGAGTGAAGTTCAGCAGGAGCGCAGACGCCCCCGCGTACA |
| | AGCAGGGCCAGAACCAGCTCTATAACGAGCTCAATCTAGG |
| | ACGAAGAGAGGAGTACGATGTTTTGGACAAGAGACGTGGC |
| | CGGGACCCTGAGATGGGGGGAAAGCCGAGAAGGAAGAACC |
| | CTCAGGAAGGCCTGTACAATGAACTGCAGAAAGATAAGAT |
| | GGCGGAGGCCTACAGTGAGATTGGGATGAAAGGCGAGCGC |
| | CGGAGGGGCAAGGGGCACGATGGCCTTTACCAGGGTCTCA |
| | GTACAGCCACCAAGGACACCTACGACGCCCTTCACATGCA |
| | GGCCCTGCCCCCTCGCTAA |
| 38 | ACCACGACGCCAGCGCCGCGACCACCAACACCGGCGCCCA |
| | CCATCGCGTCGCAGCCCCTGTCCCTGCGCCCAGAGGCGTG |
| | CCGGCCAGCGGCGGGGGGCGCAGTGCACACGAGGGGGCTG |
| | GACTTCGCCTGTGATATCTACATCTGGGCGCCCTTGGCCG |
| | GGACTTGTGGGGTCCTTCTCCTGTCACTGGTTATCACCCT |
| | TTACTGCAAACGGGGCAGAAAGAAACTCCTGTATATATTC |
| | AAACAACCATTTATGAGACCAGTACAAACTACTCAAGAGG |
| | AAGATGGCTGTAGCTGCCGATTTCCAGAAGAAGAAGAAGG |
| | AGGATGTGAACTGAGAGTGAAGTTCAGCAGGAGCGCAGAC |
| | GCCCCCGCGTACAAGCAGGGCCAGAACCAGCTCTATAACG |
| | AGCTCAATCTAGGACGAAGAGAGGAGTACGATGTTTTGGA |
| | CAAGAGACGTGGCCGGGACCCTGAGATGGGGGGAAAGCCG |
| | AGAAGGAAGAACCCTCAGGAAGGCCTGTACAATGAACTGC |
| | AGAAAGATAAGATGGCGGAGGCCTACAGTGAGATTGGGAT |
| | GAAAGGCGAGCGCCGGAGGGGCAAGGGGCACGATGGCCTT |
| | TACCAGGGTCTCAGTACAGCCACCAAGGACACCTACGACG |
| | CCCTTCACATGCAGGCCCTGCCCCCTCGCTAA |

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some embodiments, the nucleic acid may encode additional amino acid sequences that do not affect the function of the CAR and which may or may not be translated upon expression of the nucleic acid by a host cell.

The nucleic acids of an embodiment of the present disclosure may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green et al., supra. The nucleic acids can be constructed based on chemical synthesis and/or enzymatic ligation reactions using procedures known in the art. See, for example, Green et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the present disclosure can be purchased from companies, such as Macromolecular Resources (Fort Collins, CO) and Synthegen (Houston, TX).

The nucleic acid can comprise any isolated or purified nucleotide sequence which encodes any of the CARs or functional portions or functional variants thereof. Alternatively, the nucleotide sequence can comprise a nucleotide sequence which is degenerate to any of the sequences or a combination of degenerate sequences.

An embodiment of the present disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive CARs. Itis generally appreciated that conditions can be rendered more stringent by the addition of increasing amounts of formamide.

The present disclosure also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In an embodiment, the nucleic acids of the present disclosure can be incorporated into a recombinant expression vector. In this regard, an embodiment of the present disclosure provides recombinant expression vectors comprising any of the nucleic acids of the present disclosure. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally-occurring as a whole.

However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector of the present disclosure can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, MD), the pBluescript series (Stratagene, LaJolla, CA), the pET series (Novagen, Madison, WI), the pGEX series (Phannacia Biotech, Uppsala, Sweden), and the pEX series (Clontech, Palo Alto, CA).

Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM, and pMAMneo (Clontech). The recombinant expression vector may be a viral vector, e.g., a retroviral vector or a lentiviral vector, an oncolytic viral vector, an adenoviral vector, an adeno-associated viral (AAV) vector, and a virus-like particle (VLP).

In an embodiment, the recombinant expression vectors of the present disclosure can be prepared using standard recombinant DNA techniques described in, for example, Green et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may also comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR (including functional portions and functional variants thereof), or to the nucleotide sequence which is complementary to or which hybridizes to the nucleotide sequence encoding the CAR. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

Included in the scope of the present disclosure are conjugates, e.g., bioconjugates, comprising any of the inventive CARs (including any of the functional portions or variants thereof), nucleic acids, recombinant expression vectors, host cells, or populations of host cells. Conjugates, as well as methods of synthesizing conjugates in general, are known in the art.

An embodiment of the present disclosure further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant CAR, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). In some embodiments, the host cell may be a T cell. For purposes herein, the T cell can be any T cell, such as a cultured T cell, e.g., a primary T cell, or a T cell from a cultured T cell line, e.g., Jurkat, SupTl, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CDS⁺ double positive T cells, CD4+ helper T cells, e.g., Th₁ and Th₂ cells, CDS⁺ T cells (e.g., cytotoxic T cells), tumor infiltrating cells, memory T cells, naïve T cells, and the like. The T cell may be a CDS⁺ T cell or a CD4⁺ T cell.

In other embodiments, the host cell may be a Natural Killer (NK) cell. In other embodiments, the host cell may be a cytotoxic T lymphocyte (CTL). In other embodiments, the host cell may be a regulatory T cell. In other embodiments, the host cell may be a Natural Killer T cell (NKT).

Also provided by an embodiment of the present disclosure is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the present disclosure, the population of cells is a clonal population comprising host cells comprising a recombinant expression vector as described herein.

CARs (including functional portions and variants thereof), nucleic acids, recombinant expression vectors, and host cells (including populations thereof), all of which are collectively referred to as “CAR materials” hereinafter, can be isolated and/or purified. The term “isolated” as used herein means having been removed from its natural environment. The term “purified” or “isolated” does not require absolute purity or isolation; rather, it is intended as a relative term. Thus, for example, a purified (or isolated) host cell preparation is one in which the host cell is more pure than cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

The CAR materials can be formulated into a composition, such as a pharmaceutical composition. In this regard, an embodiment of the present disclosure provides a pharmaceutical composition comprising any of the CAR materials described herein, and a pharmaceutically acceptable carrier. The inventive pharmaceutical compositions containing any of the CAR materials can comprise more than one inventive CAR material, e.g.; a CAR and a nucleic acid, or two or more different CARs. Alternatively, the pharmaceutical composition can comprise an inventive CAR material in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In a preferred embodiment, the pharmaceutical composition comprises the inventive host cell or populations thereof.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which has no detrimental side effects or toxicity under the conditions of use.

The choice of carrier will be determined in part by the particular inventive CAR material, as well as by the particular method used to administer the inventive CAR material. Accordingly, there are a variety of suitable formulations of the pharmaceutical composition of the present disclosure. Methods for preparing administrable (e.g., parenterally administrable) compositions are known or apparent to those skilled in the art and are described in more detail in, for example, Remington: The Science and Practice of Pharmacy, Pharmaceutical Press; 22nd ed. (2012).

The CAR materials may be administered in any suitable manner. Preferably, the CAR materials are administered by injection, (e.g., subcutaneously, intravenously, intratumorally, intraarterially, intramuscularly, intradermally, interperitoneally, or intrathecally). Preferably, the CAR materials are administered intravenously.

A suitable pharmaceutically acceptable carrier for the inventive CAR material for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an embodiment, the pharmaceutically acceptable carrier is supplemented with human serum albumen.

An “effective amount” or “an amount effective to treat” refers to a dose that is adequate to prevent or treat cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the CAR materials in each or various rounds of administration. By way of example and not intending to limit the present disclosure, when the inventive CAR material is a host cell, an exemplary dose of host cells may be a minimum of one million cells (1×10⁶ cells/dose). In some embodiments, a subject of the present disclosure (e.g., a human subject) is administered a dose of CAR⁺ cells that ranges from approximately 1.0×10⁵ to approximately 5.0×10⁶ CAR⁺ cells per kilogram of body weight of the subject, or from approximately 4.0×10⁵ to 1.0×10⁹ total CAR⁺ cells.

For purposes of the present disclosure, the amount or dose of the CAR material administered should be sufficient to effect a therapeutic or prophylactic response in the subject or animal over a reasonable time frame. For example, the dose of the inventive CAR material should be sufficient to bind to antigen, or detect, treat or prevent cancer in a period of from about 2 hours or longer, e.g., about 12 to about 24 or more hours, from the time of administration. In certain embodiments, the time period could be even longer. The dose will be determined by the efficacy of the particular inventive CAR material and the condition of the animal (e.g., human), as well as the body weight of the animal (e.g., human) to be treated.

For purposes of the present disclosure, an assay, which comprises, for example, comparing the extent to which target cells are lysed and/or IFN-γ is secreted by T cells expressing the inventive CAR upon administration of a given dose of such T cells to a mammal, among a set of mammals of which is each given a different dose of the T cells, could be used to determine a starting dose to be administered to a mammal. The extent to which target cells are lysed and/or IFN-γ is secreted upon administration of a certain dose can be assayed by methods known in the art.

When the CAR materials are administered with one or more additional therapeutic agents, one or more additional therapeutic agents can be coadministered to the mammal. By “coadministering” is meant administering one or more additional therapeutic agents and the CAR materials sufficiently close in time such that the CAR materials can enhance the effect of one or more additional therapeutic agents, or vice versa. In this regard, the CAR materials can be administered first and the one or more additional therapeutic agents can be administered second, or vice versa. Alternatively, the CAR materials and the one or more additional therapeutic agents can be administered simultaneously. An exemplary therapeutic agent that can be co-administered with the CAR materials is IL-2. It is believed that IL-2 enhances the therapeutic effect of the CAR materials. As described herein, administration of allogeneic cells that includes a chimeric receptor, which can include the CAR materials described herein, can be administered together in a therapy (e.g., as part of a therapeutic treatment regimen or multi-component cellular therapy product) with an immune-modulating cell therapy product (e.g., any of the a regulatory T cell therapies and/or alloHSCTs described herein), including but not limited to co-administration, sequential administration, concomitant administration of (a) allogeneic cells that includes a chimeric receptor and (b) an immune-modulating cell therapy product. As an illustrative non-limiting example, any of the Tregs described herein and any of the HSPCs described herein can be administered on day 0 of a treatment regimen, then cells that include a chimeric receptor (e.g., any of the CAR materials described herein) can be administered on day 2. In a further illustration, cells that include a chimeric receptor can be administered together with any of the Tcons described herein on the same day (e.g., Day 2), such as separate administration of the cells that include the chimeric receptor after administration of the Tcons.

It is contemplated that the CAR materials can be used in methods of treating or preventing a disease in a mammal. Without being bound to a particular theory or mechanism, the CAR materials have biological activity, e.g., ability to recognize antigen, e.g., BCMA or one or both of CDI9 and CD22, such that the CAR when expressed by a cell is able to mediate an immune response against the cell expressing the antigen, e.g., BCMA or one or both of CD19 and CD22, for which the CAR has dual specificity. In this regard, an embodiment of the present disclosure provides a method of treating or preventing cancer in a mammal, comprising administering to the mammal any of the CARs, the nucleic acids, the recombinant expression vectors, the host cells, the population of cells, and/or the pharmaceutical compositions of the present disclosure in an amount effective to treat or prevent cancer in the mammal.

An embodiment of the present disclosure further comprises lymphodepleting the mammal prior to administering the CAR materials. Examples of lymphodepletion include, but may not be limited to, nonmyeloablative lymphodepleting chemotherapy, myeloablative lymphodepleting chemotherapy, total body irradiation, etc. Conditioning regimens, including myeloablative conditioning, is described further herein.

For purposes of the inventive methods, wherein host cells or populations of cells are administered, the cells can be cells that are allogeneic or autologous to the mammal. Preferably, the cells are autologous to the mammal.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the inventive methods, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia (CLL), chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma), lymphoma, malignant mesothelioma, mastocytoma, melanoma, multiple myeloma, nasopharynx cancer, non-Hodgkin lymphoma, B-chronic lymphocytic leukemia, B-precursor acute lymphoblastic leukemia (B-ALL), pre-B cell precursor acute lymphoblastic leukemia (BCP-ALL), B cell lymphoma, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, and ureter cancer. Preferably, the cancer is a hematological malignancy (e.g., leukemia or lymphoma, including but not limited to Hodgkin lymphoma, non-Hodgkin lymphoma, CLL, acute lymphocytic cancer, acute myeloid leukemia, B-chronic lymphocytic leukemia, hairy cell leukemia, acute lymphocytic leukemia (ALL) (also referred to as “acute lymphoblastic leukemia”), B-ALL, BCP-ALL, B cell lymphoma, Burkitt's lymphoma, and myeloma, including but not limited to multiple myeloma (MM)). In some embodiments, the cancer may be characterized by the expression of BCMA or one or both of CD22 and CD19, and may be a hematological malignancy that is characterized by the expression of BCMA or one or both of CD19 and CD22.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment of the present disclosure provides a use of the inventive CARs, nucleic acids, recombinant expression vectors, host cells, populations of cells, or pharmaceutical compositions, for the treatment or prevention of cancer in a mammal.

Methods of testing a CAR for the ability to recognize target cells and for antigen specificity are known in the art. For instance, Clay et al., J. Immunol., 163:507-513 (1999), teaches methods of measuring the release of cytokines (e.g., interferon-y, granulocyte/monocyte colony stimulating factor (GM-CSF), tumor necrosis factor a (TNF-a) or interleukin 2 (IL-2)). In addition, CAR function can be evaluated by measurement of cellular cytoxicity, as described in Zhao et al., J. Immunol., 174:4415-4423 (2005).

### Allogeneic Hematopoietic Stem Cell Transplantation

Various embodiments of the present disclosure provide compositions, multi-component pharmaceutical treatments, multi-component cellular therapy products, cell populations, solutions, formulations, kits, and/or methods relating to improved allogeneic hematopoietic stem cell transplantation (alloHSCT) that includes transplantation of distinct cell populations that are enriched for hematopoietic stem and progenitor cells (HSPCs), regulatory T-cells (Tregs), and conventional T-cells (Tcons), e.g., for combined use with allogeneic cells that include a chimeric receptor, e.g., a TCR or CAR-T cell (“CAR materials” described herein).

Standard alloHSCT is the transplantation of multipotent hematopoietic stem and progenitor cells (HSPCs), usually derived from donor bone marrow, peripheral blood, or umbilical cord blood, into a recipient. The recipient can be subjected to myeloablative conditioning, which kills hematopoietic cells including tumor cells and host immune cells. The HSPCs transplanted into the recipient then reconstitutes the hematopoietic compartment. HSCT can be useful as a treatment for cancer due to the ability of donor T cells to exert anti-tumor effects, referred to as graft versus tumor (GVT). In patients with hematologic malignancies that are refractory to chemotherapy, HSCT is associated with improved survival.

Surprisingly, recipients of the improved alloHSCT of the present disclosure who received a single agent graft versus host disease (GVHD) prophylactic agent, such as tacrolimus, had significantly better clinical outcomes than existing standard alloHSCT regimens and standards of care. These recipients experience improved clinical outcomes including, for example, increased overall survival, increased relapse-free survival, increased GVHD- and relapse-free survival (GRFS), more rapid and/or complete engraftment of various hematopoietic components (e.g., neutrophils, platelets, T cells, B cells), improved donor chimerism (e.g., T cell chimerism), decreased relapse, decreased primary graft failure, decreased secondary graft failure, decreased treatment-associated mortality, reduced acute and/or chronic GVHD, and shorter time to discharge from hospital following the single agent GVHD prophylactic agent, such as tacrolimus

Although alloHSCT is associated with improved survival in patients with hematologic malignancies that are refractory to chemotherapy, some subjects treated with existing standard alloHSCT regimens exhibit cancer relapse, and a number of complications can limit the efficacy of standard alloHSCT. The effectiveness of standard alloHSCT can be limited by, for example, primary graft failure, secondary graft failure, limited or slow engraftment of various hematopoietic components (e.g., neutrophils, platelets, T cells, or B cells), and limited donor chimerism (e.g., T cell chimerism). Additionally, standard alloHSCT can cause treatment-associated morality or toxicity, for example, However, donor T cells can also attack non-tumor host cells, resulting in graft versus host disease (GVHD). GVHD is a major source of post-HCT complications and can be fatal. Management of GVHD can require immunosuppressive therapy or cytotoxic mediations, which can cause toxicity, increase susceptibility to infection, and/or blunt anti-tumor immunity. The early morbidity and mortality associated with acute graft versus host disease (aGVHD; which occurs within the first 100 days post-transplant) is a major factor limiting the success of HCT, as is the long-term morbidity associated with chronic GVHD (cGVHD). GVHD is a risk for both HLA-matched and HLA-mismatched transplantations. GVHD can occur even if the donor and recipient are HLA-matched, because the immune system can still recognize other differences between in the donor tissues.

Both GVT and GVHD are largely mediated by conventional T cells (Tcons), which mount immune responses upon recognition of cognate antigen by T cell receptors. Depleting T cells from hematopoietic stem cell transplantation (HCT) grafts can reduce GVHD, but can also result in reduced GVT and increased likelihood of cancer relapse. Besides Tcons, Tregs are an additional subset of T cells that negatively regulate inflammation and that promote immune tolerance. Tregs can prevent or reduce GVHD through their negative regulation of inflammation, including, for example, inflammation elicited by donor Tcons when they recognize recipient antigens.

Provided herein are compositions and methods for improved alloHSCT, comprising administering to a subject certain cell populations that comprise populations of cells, including a first population of CD45⁺ cells that comprise, at least, HSPCs, a second population of CD45⁺ cells that comprise, at least Tregs, and a third population of CD45⁺ cells that comprises, at least, Tcons. In some embodiments, the second population of CD45⁺ cells is also referred to a cell population enriched for Tregs. Without wishing to be bound by theory, administering the second population of CD45⁺ cells reduces the incidence and/or severity of GVHD, while administering the third population of CD45⁺ cells, which comprises Tcons, enhances GVT. Thus, embodiments of the present disclosure provide a provides compositions, multi-component pharmaceutical treatments, multi-component cellular therapy products, cell populations, solutions, formulations, kits, and/or methods for administering, at least, both populations of T cells, to enhance GVT while minimizing GVHD. Accordingly, the compositions, multi-component pharmaceutical treatments, multi-component cellular therapy products, cell populations, solutions, formulations, kits, and/or methods disclosed herein can retain the graft-versus-tumor (GVT) effects of alloHSCT administered to a subject having a cancer (e.g., a hematologic cancer), while preventing or reducing graft versus host disease (GVHD) in the subject. In some embodiments, two or more populations of cells are administered at different times, for example, first population of CD45⁺ cells that comprises, at least, HSPCs and the cell population enriched for Tregs can be administered prior to the third population of CD45⁺ cells that comprises, at least, Tcons.

### Cell Populations

Embodiments of the present disclosure provide a multi-component pharmaceutical treatment or multi-component cellular therapy product to be administered to a human subject in need thereof. In some embodiments, the multi-component treatment comprises (a) a solution comprising a first population of CD45⁺ cells comprising hematopoietic stem and progenitor cells (HSPCs) and granulocytes wherein at most about 10% of the first population of CD45⁺ cells comprise granulocytes; (b) a solution comprising a population of cells enriched for regulatory T cells (Tregs); (c) a solution comprising a third population of CD45⁺ cells wherein the third population of CD45⁺ cells comprise at least about 20% CD3⁺ conventional T cells (Tcons), at least about 10% monocytes, and at least about 10% granulocytes; and (d) a solution comprising one or more doses of a graft vs host disease (GVHD) prophylactic agent, e.g., tacrolimus. In various embodiments, the HSPCs are CD34⁺. In some embodiments, the multi-component cellular therapy product comprises a) a first single dose transfer bag comprising a first population of isolated CD45⁺ cells comprising a dose of approximately 1.0×10⁵ to approximately 1.0×10⁸ CD34⁺ hematopoietic stem and progenitor cells (HSPCs) per kilogram of body weight of a human subject receiving the product, from approximately 1.5×10⁷ to approximately 1.5×10¹⁰ HSPCs, or from approximately 5.0×10⁵ to approximately 5.0×10⁸ HSPCs, wherein the first population of CD45⁺ cells is formulated with an excipient at a neutral pH; b) a second single dose transfer bag comprising a second population of isolated CD45⁺ cells comprising a dose of approximately 1.0×10⁵ to approximately 2.0×10⁷ fresh CD4⁺CD25⁺CD127^{dim} regulatory T cells (Tregs) per kilogram of body weight of the human subject receiving the product, from approximately 1.5×10⁷ to approximately 3.0×10⁹ fresh Tregs, or from approximately 5.0×10⁵ to approximately 1.0×10⁸ fresh Tregs, wherein the second population of isolated CD45⁺ cells is formulated with an excipient at a neutral pH; and c) a third single dose transfer bag comprising a third population of isolated CD45⁺ cells comprising a dose of approximately 1.0×10⁵ to approximately 4.0×10⁷ conventional CD3⁺ T cells (Tcons) per kilogram of body weight of the human subject receiving the product, from approximately 1.5×10⁷ to approximately 6.0×10⁹ Tcons, or from approximately 5.0×10⁵ to approximately 2.0×10⁸ Tcons, wherein the third population of isolated CD45⁺ cells is formulated with an excipient at a neutral pH, wherein the excipient comprises one more cryoprotectants.

In some embodiments, the first population of CD45⁺ cells comprises CD34⁺ hematopoietic stem and progenitor cells (HSPCs). As used herein, a first population of CD45⁺ cells and a population of CD34⁺ hematopoietic stem and progenitor cells (HSPCs) may be used interchangeably. In other embodiments, the first population of CD45⁺ cells comprises at least one dose of HSPCs. In some embodiments, the first population of CD45⁺ cells comprising HSPCs, or comprising at least one dose of HSPCs, comprises from approximately 1.0×10⁵ to approximately 5.0×10⁸ HSPCs per kilogram of body weight of the human subject, from approximately 1.0×10⁵ to approximately 1.0×10⁸ HSPCs per kilogram of body weight of the human subject, or from approximately 5.0×10⁵ to approximately 2.0×10⁷ HSPCs per kilogram of body weight of the human subject. In some embodiments, the first population of CD45⁺ cells comprising HSPCs, or comprising at least one dose of HSPCs, comprises approximately 1.0×10⁵ or more HSPCs per kilogram of body weight of the human subject, approximately 1.5×10⁵ or more HSPCs per kilogram of body weight of the human subject, approximately 2.0×10⁵ or more HSPCs per kilogram of body weight of the human subject, approximately 2.5×10⁵ or more HSPCs per kilogram of body weight of the human subject, approximately 3.0×10⁵ or more HSPCs per kilogram of body weight of the human subject, approximately 3.5×10⁵ or more HSPCs per kilogram of body weight of the human subject, approximately 4.0×10⁵ or more HSPCs per kilogram of body weight of the human subject, approximately 4.5×10⁵ or more HSPCs per kilogram of body weight of the human subject, approximately 5.0×10⁵ or more HSPCs per kilogram of body weight of the human subject, approximately 5.5×10⁵ or more HSPCs per kilogram of body weight of the human subject, approximately 6.0×10⁵ or more HSPCs per kilogram of body weight of the human subject, approximately 6.5×10⁵ or more HSPCs per kilogram of body weight of the human subject, approximately 7.0×10⁵ or more HSPCs per kilogram of body weight of the human subject, approximately 7.5×10⁵ or more HSPCs per kilogram of body weight of the human subject, approximately 8.0×10⁵ or more HSPCs per kilogram of body weight of the human subject, approximately 8.5×10⁵ or more HSPCs per kilogram of body weight of the human subject, approximately 9.0×10⁵ or more HSPCs per kilogram of body weight of the human subject, approximately 9.5×10⁵ or more HSPCs per kilogram of body weight of the human subject, approximately 1.0×10⁶ or more HSPCs per kilogram of body weight of the human subject, approximately 1.25×10⁶ or more HSPCs per kilogram of body weight of the human subject, approximately 1.5×10⁶ or more HSPCs per kilogram of body weight of the human subject, approximately 1.75×10⁶ or more HSPCs per kilogram of body weight of the human subject, approximately 2.0×10⁶ or more HSPCs per kilogram of body weight of the human subject, approximately 2.25×10⁶ or more HSPCs per kilogram of body weight of the human subject, approximately 2.5×10⁶ or more HSPCs per kilogram of body weight of the human subject, approximately 2.75×10⁶ or more HSPCs per kilogram of body weight of the human subject, approximately 3.0×10⁶ or more HSPCs per kilogram of body weight of the human subject, approximately 3.25×10⁶ or more HSPCs per kilogram of body weight of the human subject, approximately 3.5×10⁶ or more HSPCs per kilogram of body weight of the human subject, approximately 3.75×10⁶ or more HSPCs per kilogram of body weight of the human subject, approximately 4.0×10⁶ or more HSPCs per kilogram of body weight of the human subject, approximately 4.25×10⁶ or more HSPCs per kilogram of body weight of the human subject, approximately 4.5×10⁶ or more HSPCs per kilogram of body weight of the human subject, approximately 4.75×10⁶ or more HSPCs per kilogram of body weight of the human subject, approximately 5.0×10⁶ or more HSPCs per kilogram of body weight of the human subject, approximately 5.25×10⁶ or more HSPCs per kilogram of body weight of the human subject, approximately 5.5×10⁶ or more HSPCs per kilogram of body weight of the human subject, approximately 5.75×10⁶ or more HSPCs per kilogram of body weight of the human subject, approximately 6.0×10⁶ or more HSPCs per kilogram of body weight of the human subject, approximately 6.25×10⁶ or more HSPCs per kilogram of body weight of the human subject, approximately 6.5×10⁶ or more HSPCs per kilogram of body weight of the human subject, approximately 6.75×10⁶ or more HSPCs per kilogram of body weight of the human subject, approximately 7.0×10⁶ or more HSPCs per kilogram of body weight of the human subject, approximately 7.25×10⁶ or more HSPCs per kilogram of body weight of the human subject, approximately 7.5×10⁶ or more HSPCs per kilogram of body weight of the human subject, approximately 7.75×10⁶ or more HSPCs per kilogram of body weight of the human subject, approximately 8.0×10⁶ or more HSPCs per kilogram of body weight of the human subject, approximately 8.25×10⁶ or more HSPCs per kilogram of body weight of the human subject, approximately 8.5×10⁶ or more HSPCs per kilogram of body weight of the human subject, approximately 8.75×10⁶ or more HSPCs per kilogram of body weight of the human subject, approximately 9.0×10⁶ or more HSPCs per kilogram of body weight of the human subject, approximately 9.25×10⁶ or more HSPCs per kilogram of body weight of the human subject, approximately 9.5×10⁶ or more HSPCs per kilogram of body weight of the human subject, approximately 9.75×10⁶ or more HSPCs per kilogram of body weight of the human subject, approximately 1.0×10⁷ or more HSPCs per kilogram of body weight of the human subject, approximately 1.25×10⁷ or more HSPCs per kilogram of body weight of the human subject, approximately 1.5×10⁷ or more HSPCs per kilogram of body weight of the human subject, approximately 1.75×10⁷ or more HSPCs per kilogram of body weight of the human subject, approximately 2.0×10⁷ or more HSPCs per kilogram of body weight of the human subject, approximately 2.25×10⁷ or more HSPCs per kilogram of body weight of the human subject, approximately 2.5×10⁷ or more HSPCs per kilogram of body weight of the human subject, approximately 2.75×10⁷ or more HSPCs per kilogram of body weight of the human subject, approximately 3.0×10⁷ or more HSPCs per kilogram of body weight of the human subject, approximately 3.25×10⁷ or more HSPCs per kilogram of body weight of the human subject, approximately 3.5×10⁷ or more HSPCs per kilogram of body weight of the human subject, approximately 3.75×10⁷ or more HSPCs per kilogram of body weight of the human subject, approximately 4.0×10⁷ or more HSPCs per kilogram of body weight of the human subject, approximately 4.25×10⁷ or more HSPCs per kilogram of body weight of the human subject, approximately 4.5×10⁷ or more HSPCs per kilogram of body weight of the human subject, approximately 4.75×10⁷ or more HSPCs per kilogram of body weight of the human subject, approximately 5.0×10⁷ or more HSPCs per kilogram of body weight of the human subject, approximately 5.25×10⁷ or more HSPCs per kilogram of body weight of the human subject, approximately 5.5×10⁷ or more HSPCs per kilogram of body weight of the human subject, approximately 5.75×10⁷ or more HSPCs per kilogram of body weight of the human subject, approximately 6.0×10⁷ or more HSPCs per kilogram of body weight of the human subject, approximately 6.25×10⁷ or more HSPCs per kilogram of body weight of the human subject, approximately 6.5×10⁷ or more HSPCs per kilogram of body weight of the human subject, approximately 6.75×10⁷ or more HSPCs per kilogram of body weight of the human subject, approximately 7.0×10⁷ or more HSPCs per kilogram of body weight of the human subject, approximately 7.25×10⁷ or more HSPCs per kilogram of body weight of the human subject, approximately 7.5×10⁷ or more HSPCs per kilogram of body weight of the human subject, approximately 7.75×10⁷ or more HSPCs per kilogram of body weight of the human subject, approximately 8.0×10⁷ or more HSPCs per kilogram of body weight of the human subject, approximately 8.25×10⁷ or more HSPCs per kilogram of body weight of the human subject, approximately 8.5×10⁷ or more HSPCs per kilogram of body weight of the human subject, approximately 8.75×10⁷ or more HSPCs per kilogram of body weight of the human subject, approximately 9.0×10⁷ or more HSPCs per kilogram of body weight of the human subject, approximately 9.25×10⁷ or more HSPCs per kilogram of body weight of the human subject, approximately 9.5×10⁷ or more HSPCs per kilogram of body weight of the human subject, approximately 9.75×10⁷ or more HSPCs per kilogram of body weight of the human subject, approximately 1.0×10⁸ or more HSPCs per kilogram of body weight of the human subject, approximately 1.25×10⁸ or more HSPCs per kilogram of body weight of the human subject, approximately 1.5×10⁸ or more HSPCs per kilogram of body weight of the human subject, approximately 1.75×10⁸ or more HSPCs per kilogram of body weight of the human subject, approximately 2.0×10⁸ or more HSPCs per kilogram of body weight of the human subject, approximately 2.25×10⁸ or more HSPCs per kilogram of body weight of the human subject, approximately 2.5×10⁸ or more HSPCs per kilogram of body weight of the human subject, approximately 2.75×10⁸ or more HSPCs per kilogram of body weight of the human subject, approximately 3.0×10⁸ or more HSPCs per kilogram of body weight of the human subject, approximately 3.25×10⁸ or more HSPCs per kilogram of body weight of the human subject, approximately 3.5×10⁸ or more HSPCs per kilogram of body weight of the human subject, approximately 3.75×10⁸ or more HSPCs per kilogram of body weight of the human subject, approximately 4.0×10⁸ or more HSPCs per kilogram of body weight of the human subject, approximately 4.25×10⁸ or more HSPCs per kilogram of body weight of the human subject, approximately 4.5×10⁸ or more HSPCs per kilogram of body weight of the human subject, approximately 4.75×10⁸ or more HSPCs per kilogram of body weight of the human subject, or approximately 5.0×10⁸ or more HSPCs per kilogram of body weight of the human subject. In some embodiments, the body weight of the human subject is actual body weight of the human subject. In other embodiments, the body weight of the human subject is ideal actual body weight of the human subject.

In embodiments, the first population of CD45⁺ cells comprises at least about 0.5% granulocytes, at least about 1% granulocytes, at most about 5% granulocytes, at most about 3% granulocytes, at most about 3% monocytes, at most about 2% monocytes, at most about 0.5% lymphocytes, at most about 2% lymphocytes, at least about 15% granulocytes, at least about 20% granulocytes, at most about 35% granulocytes, at most about 30% granulocytes, at most about 25% granulocytes, at least about 15% monocytes, at least about 20% monocytes, at most about 35% monocytes, at most about 30% monocytes, at most about 25% monocytes, at least about 0.5% NK cells, and or at least about 2% NK cells. In various embodiments, the first population of CD45⁺ cells, the population of cells enriched for Tregs, and the third population of CD45⁺ cells are obtained from a single donor. In some embodiments, the first population of CD45⁺ cells, the population of cells enriched for Tregs, and/or the third population of CD45⁺ cells is allogeneic relative to the human subject. In embodiments, the first population of CD45⁺ cells, the population of cells enriched for Tregs, and/or the third population of CD45⁺ cells is obtained from a donor that is HLA-matched relative to the human subject. In various embodiments, the first population of CD45⁺ cells, the population of cells enriched for Tregs, and/or the third population of CD45⁺ cells is obtained from a donor that is HLA-mismatched relative to the human subject. In some embodiments, the first population of CD45⁺ cells, the population of cells enriched for Tregs, and/or the third population of CD45⁺ cells is obtained from a donor that is haploidentical relative to the human subject.

In some embodiments, the Tregs are CD4⁺ CD25⁺ CD127^{dim} or CD4⁺ FOXP3⁺. In some embodiments, the Tregs are CD4⁺CD25⁺CD127^{dim} and are also FOXP3⁺. In some cases, the population of cells enriched for Tregs comprises CD45⁺ cells, e.g., more than about 90% of the CD45⁺ cells are Tregs. In some embodiments, the second population of CD45⁺ cells comprises CD4⁺CD25⁺CD127^{dim} regulatory T cells (Tregs). As used herein, a second population of CD45⁺ cells, a population of cells enriched for Tregs, and a population of CD4⁺CD25⁺CD127^{dim} regulatory T cells (Tregs) may be used interchangeably. In other embodiments, the second population of CD45⁺ cells comprises one or more doses of Tregs. In various embodiments, the population of cells enriched for Tregs, or the second population of CD45⁺ cells comprising at least one dose of Tregs, comprises from approximately 1.0×10⁵ to approximately 1.0×10⁸ Tregs per kilogram of body weight of the human subject, from approximately 1.0×10⁵ to approximately 2.0×10⁷ Tregs per kilogram of body weight of the human subject, from approximately 1.0×10⁵ to approximately 1.0×10⁷ Tregs per kilogram of body weight of the human subject, or from approximately 5.0×10⁵ to approximately 4.0×10⁶ Tregs per kilogram of body weight of the human subject. In some embodiments, the population of cells enriched for Tregs, or the second population of CD45⁺ cells comprising at least one dose of Tregs, comprises approximately 1.0×10⁵ or more Tregs per kilogram of body weight of the human subject, approximately 1.5×10⁵ or more Tregs per kilogram of body weight of the human subject, approximately 2.0×10⁵ or more Tregs per kilogram of body weight of the human subject, approximately 2.5×10⁵ or more Tregs per kilogram of body weight of the human subject, approximately 3.0×10⁵ or more Tregs per kilogram of body weight of the human subject, approximately 3.5×10⁵ or more Tregs per kilogram of body weight of the human subject, approximately 4.0×10⁵ or more Tregs per kilogram of body weight of the human subject, approximately 4.5×10⁵ or more Tregs per kilogram of body weight of the human subject, approximately 5.0×10⁵ or more Tregs per kilogram of body weight of the human subject, approximately 5.5×10⁵ or more Tregs per kilogram of body weight of the human subject, approximately 6.0×10⁵ or more Tregs per kilogram of body weight of the human subject, approximately 6.5×10⁵ or more Tregs per kilogram of body weight of the human subject, approximately 7.0×10⁵ or more Tregs per kilogram of body weight of the human subject, approximately 7.5×10⁵ or more Tregs per kilogram of body weight of the human subject, approximately 8.0×10⁵ or more Tregs per kilogram of body weight of the human subject, approximately 8.5×10⁵ or more Tregs per kilogram of body weight of the human subject, approximately 9.0×10⁵ or more Tregs per kilogram of body weight of the human subject, approximately 9.5×10⁵ or more Tregs per kilogram of body weight of the human subject, approximately 1.0×10⁶ or more Tregs per kilogram of body weight of the human subject, approximately 1.25×10⁶ or more Tregs per kilogram of body weight of the human subject, approximately 1.5×10⁶ or more Tregs per kilogram of body weight of the human subject, approximately 1.75×10⁶ or more Tregs per kilogram of body weight of the human subject, approximately 2.0×10⁶ or more Tregs per kilogram of body weight of the human subject, approximately 2.25×10⁶ or more Tregs per kilogram of body weight of the human subject, approximately 2.5×10⁶ or more Tregs per kilogram of body weight of the human subject, approximately 2.75×10⁶ or more Tregs per kilogram of body weight of the human subject, approximately 3.0×10⁶ or more Tregs per kilogram of body weight of the human subject, approximately 3.25×10⁶ or more Tregs per kilogram of body weight of the human subject, approximately 3.5×10⁶ or more Tregs per kilogram of body weight of the human subject, approximately 3.75×10⁶ or more Tregs per kilogram of body weight of the human subject, approximately 4.0×10⁶ or more Tregs per kilogram of body weight of the human subject, approximately 4.25×10⁶ or more Tregs per kilogram of body weight of the human subject, approximately 4.5×10⁶ or more Tregs per kilogram of body weight of the human subject, approximately 4.75×10⁶ or more Tregs per kilogram of body weight of the human subject, approximately 5.0×10⁶ or more Tregs per kilogram of body weight of the human subject, approximately 5.25×10⁶ or more Tregs per kilogram of body weight of the human subject, approximately 5.5×10⁶ or more Tregs per kilogram of body weight of the human subject, approximately 5.75×10⁶ or more Tregs per kilogram of body weight of the human subject, approximately 6.0×10⁶ or more Tregs per kilogram of body weight of the human subject, approximately 6.25×10⁶ or more Tregs per kilogram of body weight of the human subject, approximately 6.5×10⁶ or more Tregs per kilogram of body weight of the human subject, approximately 6.75×10⁶ or more Tregs per kilogram of body weight of the human subject, approximately 7.0×10⁶ or more Tregs per kilogram of body weight of the human subject, approximately 7.25×10⁶ or more Tregs per kilogram of body weight of the human subject, approximately 7.5×10⁶ or more Tregs per kilogram of body weight of the human subject, approximately 7.75×10⁶ or more Tregs per kilogram of body weight of the human subject, approximately 8.0×10⁶ or more Tregs per kilogram of body weight of the human subject, approximately 8.25×10⁶ or more Tregs per kilogram of body weight of the human subject, approximately 8.5×10⁶ or more Tregs per kilogram of body weight of the human subject, approximately 8.75×10⁶ or more Tregs per kilogram of body weight of the human subject, approximately 9.0×10⁶ or more Tregs per kilogram of body weight of the human subject, approximately 9.25×10⁶ or more Tregs per kilogram of body weight of the human subject, approximately 9.5×10⁶ or more Tregs per kilogram of body weight of the human subject, approximately 9.75×10⁶ or more Tregs per kilogram of body weight of the human subject, approximately 1.0×10⁷ or more Tregs per kilogram of body weight of the human subject, approximately 1.25×10⁷ or more Tregs per kilogram of body weight of the human subject, approximately 1.5×10⁷ or more Tregs per kilogram of body weight of the human subject, approximately 1.75×10⁷ or more Tregs per kilogram of body weight of the human subject, approximately 2.0×10⁷ or more Tregs per kilogram of body weight of the human subject, approximately 2.25×10⁷ or more Tregs per kilogram of body weight of the human subject, approximately 2.5×10⁷ or more Tregs per kilogram of body weight of the human subject, approximately 2.75×10⁷ or more Tregs per kilogram of body weight of the human subject, approximately 3.0×10⁷ or more Tregs per kilogram of body weight of the human subject, approximately 3.25×10⁷ or more Tregs per kilogram of body weight of the human subject, approximately 3.5×10⁷ or more Tregs per kilogram of body weight of the human subject, approximately 3.75×10⁷ or more Tregs per kilogram of body weight of the human subject, approximately 4.0×10⁷ or more Tregs per kilogram of body weight of the human subject, approximately 4.25×10⁷ or more Tregs per kilogram of body weight of the human subject, approximately 4.5×10⁷ or more Tregs per kilogram of body weight of the human subject, approximately 4.75×10⁷ or more Tregs per kilogram of body weight of the human subject, approximately 5.0×10⁷ or more Tregs per kilogram of body weight of the human subject, approximately 5.25×10⁷ or more Tregs per kilogram of body weight of the human subject, approximately 5.5×10⁷ or more Tregs per kilogram of body weight of the human subject, approximately 5.75×10⁷ or more Tregs per kilogram of body weight of the human subject, approximately 6.0×10⁷ or more Tregs per kilogram of body weight of the human subject, approximately 6.25×10⁷ or more Tregs per kilogram of body weight of the human subject, approximately 6.5×10⁷ or more Tregs per kilogram of body weight of the human subject, approximately 6.75×10⁷ or more Tregs per kilogram of body weight of the human subject, approximately 7.0×10⁷ or more Tregs per kilogram of body weight of the human subject, approximately 7.25×10⁷ or more Tregs per kilogram of body weight of the human subject, approximately 7.5×10⁷ or more Tregs per kilogram of body weight of the human subject, approximately 7.75×10⁷ or more Tregs per kilogram of body weight of the human subject, approximately 8.0×10⁷ or more Tregs per kilogram of body weight of the human subject, approximately 8.25×10⁷ or more Tregs per kilogram of body weight of the human subject, approximately 8.5×10⁷ or more Tregs per kilogram of body weight of the human subject, approximately 8.75×10⁷ or more Tregs per kilogram of body weight of the human subject, approximately 9.0×10⁷ or more Tregs per kilogram of body weight of the human subject, approximately 9.25×10⁷ or more Tregs per kilogram of body weight of the human subject, approximately 9.5×10⁷ or more Tregs per kilogram of body weight of the human subject, approximately 9.75×10⁷ or more Tregs per kilogram of body weight of the human subject, approximately 1.0×10⁸ or more Tregs per kilogram of body weight of the human subject. In some embodiments, the body weight of the human subject is actual body weight of the human subject. In other embodiments, the body weight of the human subject is ideal actual body weight of the human subject.

In some embodiments, the third population of CD45⁺ cells comprises conventional CD3⁺ T cells (Tcons). In other embodiments, the third population of CD45⁺ comprises at least one dose of Tcons. As used herein, a third population of CD45⁺ cells and a population of conventional CD3⁺ T cells (Tcons) may be used interchangeably. In some embodiments, the third population of CD45⁺ cells comprising Tcons, or comprising at least one dose of Tcons, comprises from approximately 1.0×10⁵ to approximately 1.0×10⁸ Tcons per kilogram of body weight of the human subject, from approximately 1.0×10⁵ to approximately 4.0×10⁷ Tcons per kilogram of body weight of the human subject, from approximately 1.0×10⁵ to approximately 1.0×10⁷ Tcons per kilogram of body weight of the human subject, or from approximately 5.0×10⁵ to approximately 5×10⁶ Tcons per kilogram of body weight of the human subject. In some embodiments, the third population of CD45⁺ cells comprising Tcons, or comprising at least one dose of Tcons, comprises approximately 1.0×10⁵ or more Tcons per kilogram of body weight of the human subject, approximately 1.5×10⁵ or more Tcons per kilogram of body weight of the human subject, approximately 2.0×10⁵ or more Tcons per kilogram of body weight of the human subject, approximately 2.5×10⁵ or more Tcons per kilogram of body weight of the human subject, approximately 3.0×10⁵ or more Tcons per kilogram of body weight of the human subject, approximately 3.5×10⁵ or more Tcons per kilogram of body weight of the human subject, approximately 4.0×10⁵ or more Tcons per kilogram of body weight of the human subject, approximately 4.5×10⁵ or more Tcons per kilogram of body weight of the human subject, approximately 5.0×10⁵ or more Tcons per kilogram of body weight of the human subject, approximately 5.5×10⁵ or more Tcons per kilogram of body weight of the human subject, approximately 6.0×10⁵ or more Tcons per kilogram of body weight of the human subject, approximately 6.5×10⁵ or more Tcons per kilogram of body weight of the human subject, approximately 7.0×10⁵ or more Tcons per kilogram of body weight of the human subject, approximately 7.5×10⁵ or more Tcons per kilogram of body weight of the human subject, approximately 8.0×10⁵ or more Tcons per kilogram of body weight of the human subject, approximately 8.5×10⁵ or more Tcons per kilogram of body weight of the human subject, approximately 9.0×10⁵ or more Tcons per kilogram of body weight of the human subject, approximately 9.5×10⁵ or more Tcons per kilogram of body weight of the human subject, approximately 1.0×10⁶ or more Tcons per kilogram of body weight of the human subject, approximately 1.25×10⁶ or more Tcons per kilogram of body weight of the human subject, approximately 1.5×10⁶ or more Tcons per kilogram of body weight of the human subject, approximately 1.75×10⁶ or more Tcons per kilogram of body weight of the human subject, approximately 2.0×10⁶ or more Tcons per kilogram of body weight of the human subject, approximately 2.25×10⁶ or more Tcons per kilogram of body weight of the human subject, approximately 2.5×10⁶ or more Tcons per kilogram of body weight of the human subject, approximately 2.75×10⁶ or more Tcons per kilogram of body weight of the human subject, approximately 3.0×10⁶ or more Tcons per kilogram of body weight of the human subject, approximately 3.25×10⁶ or more Tcons per kilogram of body weight of the human subject, approximately 3.5×10⁶ or more Tcons per kilogram of body weight of the human subject, approximately 3.75×10⁶ or more Tcons per kilogram of body weight of the human subject, approximately 4.0×10⁶ or more Tcons per kilogram of body weight of the human subject, approximately 4.25×10⁶ or more Tcons per kilogram of body weight of the human subject, approximately 4.5×10⁶ or more Tcons per kilogram of body weight of the human subject, approximately 4.75×10⁶ or more Tcons per kilogram of body weight of the human subject, approximately 5.0×10⁶ or more Tcons per kilogram of body weight of the human subject, approximately 5.25×10⁶ or more Tcons per kilogram of body weight of the human subject, approximately 5.5×10⁶ or more Tcons per kilogram of body weight of the human subject, approximately 5.75×10⁶ or more Tcons per kilogram of body weight of the human subject, approximately 6.0×10⁶ or more Tcons per kilogram of body weight of the human subject, approximately 6.25×10⁶ or more Tcons per kilogram of body weight of the human subject, approximately 6.5×10⁶ or more Tcons per kilogram of body weight of the human subject, approximately 6.75×10⁶ or more Tcons per kilogram of body weight of the human subject, approximately 7.0×10⁶ or more Tcons per kilogram of body weight of the human subject, approximately 7.25×10⁶ or more Tcons per kilogram of body weight of the human subject, approximately 7.5×10⁶ or more Tcons per kilogram of body weight of the human subject, approximately 7.75×10⁶ or more Tcons per kilogram of body weight of the human subject, approximately 8.0×10⁶ or more Tcons per kilogram of body weight of the human subject, approximately 8.25×10⁶ or more Tcons per kilogram of body weight of the human subject, approximately 8.5×10⁶ or more Tcons per kilogram of body weight of the human subject, approximately 8.75×10⁶ or more Tcons per kilogram of body weight of the human subject, approximately 9.0×10⁶ or more Tcons per kilogram of body weight of the human subject, approximately 9.25×10⁶ or more Tcons per kilogram of body weight of the human subject, approximately 9.5×10⁶ or more Tcons per kilogram of body weight of the human subject, approximately 9.75×10⁶ or more Tcons per kilogram of body weight of the human subject, approximately 1.0×10⁷ or more Tcons per kilogram of body weight of the human subject, approximately 1.25×10⁷ or more Tcons per kilogram of body weight of the human subject, approximately 1.5×10⁷ or more Tcons per kilogram of body weight of the human subject, approximately 1.75×10⁷ or more Tcons per kilogram of body weight of the human subject, approximately 2.0×10⁷ or more Tcons per kilogram of body weight of the human subject, approximately 2.25×10⁷ or more Tcons per kilogram of body weight of the human subject, approximately 2.5×10⁷ or more Tcons per kilogram of body weight of the human subject, approximately 2.75×10⁷ or more Tcons per kilogram of body weight of the human subject, approximately 3.0×10⁷ or more Tcons per kilogram of body weight of the human subject, approximately 3.25×10⁷ or more Tcons per kilogram of body weight of the human subject, approximately 3.5×10⁷ or more Tcons per kilogram of body weight of the human subject, approximately 3.75×10⁷ or more Tcons per kilogram of body weight of the human subject, approximately 4.0×10⁷ or more Tcons per kilogram of body weight of the human subject, approximately 4.25×10⁷ or more Tcons per kilogram of body weight of the human subject, approximately 4.5×10⁷ or more Tcons per kilogram of body weight of the human subject, approximately 4.75×10⁷ or more Tcons per kilogram of body weight of the human subject, approximately 5.0×10⁷ or more Tcons per kilogram of body weight of the human subject, approximately 5.25×10⁷ or more Tcons per kilogram of body weight of the human subject, approximately 5.5×10⁷ or more Tcons per kilogram of body weight of the human subject, approximately 5.75×10⁷ or more Tcons per kilogram of body weight of the human subject, approximately 6.0×10⁷ or more Tcons per kilogram of body weight of the human subject, approximately 6.25×10⁷ or more Tcons per kilogram of body weight of the human subject, approximately 6.5×10⁷ or more Tcons per kilogram of body weight of the human subject, approximately 6.75×10⁷ or more Tcons per kilogram of body weight of the human subject, approximately 7.0×10⁷ or more Tcons per kilogram of body weight of the human subject, approximately 7.25×10⁷ or more Tcons per kilogram of body weight of the human subject, approximately 7.5×10⁷ or more Tcons per kilogram of body weight of the human subject, approximately 7.75×10⁷ or more Tcons per kilogram of body weight of the human subject, approximately 8.0×10⁷ or more Tcons per kilogram of body weight of the human subject, approximately 8.25×10⁷ or more Tcons per kilogram of body weight of the human subject, approximately 8.5×10⁷ or more Tcons per kilogram of body weight of the human subject, approximately 8.75×10⁷ or more Tcons per kilogram of body weight of the human subject, approximately 9.0×10⁷ or more Tcons per kilogram of body weight of the human subject, approximately 9.25×10⁷ or more Tcons per kilogram of body weight of the human subject, approximately 9.5×10⁷ or more Tcons per kilogram of body weight of the human subject, approximately 9.75×10⁷ or more Tcons per kilogram of body weight of the human subject, approximately 1.0×10⁸ or more Tcons per kilogram of body weight of the human subject. In some embodiments, the body weight of the human subject is actual body weight of the human subject. In other embodiments, the body weight of the human subject is ideal actual body weight of the human subject.

In embodiments, the third population of CD45⁺ cells comprises at least about 0.1% CD34⁺ cells or from approximately 0.2% to approximately 20% CD34⁺ cells and/or at least about 0.1% Tregs. In various embodiments, the third population of CD45⁺ cells comprises a population of memory T cells (Tmems), e.g., Tmems that are CD3⁺ CD45RA− CD45RO⁺. In some embodiments, the population of Tmems comprises more than about 3×10⁵ Tmems per kilogram of ideal body actual or ideal body weight of the human subject. In embodiments, the population of Tmems comprises from approximately 3×10⁵ to approximately 1×10⁹ Tmems per kilogram of ideal body actual or ideal body weight of the human subject. In various embodiments, the third population of CD45⁺ cells comprises a population of invariant natural killer T cells (iNKTs), e.g., iNKTs that are CD3⁺ Vα24Jα18⁺. In some embodiments, the population of iNKTs comprises more than about 5×10² iNKTs per kilogram of ideal body actual or ideal body weight of the human subject. In embodiments, the population of iNKTs comprises from approximately 5×10² to approximately 1×10⁷ iNKTs per kilogram of ideal body actual or ideal body weight of the human subject. In some embodiments, the third population of CD45⁺ cells is co-cultured with donor cancer antigens/peptides and/or antigen-presenting cells.

In some embodiments, a cell population of the present disclosure comprises from approximately 1.0×10⁵ to approximately 5.0×10⁸ Tmem per kilogram of body weight of the human subject, from approximately 1.0×10⁵ to approximately 1.0×10⁸ Tmem per kilogram of body weight of the human subject, or from approximately 5.0×10⁵ to approximately 2.0×10⁷ Tmem per kilogram of body weight of the human subject. In some embodiments, the Tmem are present in a composition, cellular therapy product, and/or administered to a subject in an amount or dose that comprises approximately 1.0×10⁵ or more Tmem per kilogram of body weight of the human subject, approximately 1.5×10⁵ or more Tmem per kilogram of body weight of the human subject, approximately 2.0×10⁵ or more Tmem per kilogram of body weight of the human subject, approximately 2.5×10⁵ or more Tmem per kilogram of body weight of the human subject, approximately 3.0×10⁵ or more Tmem per kilogram of body weight of the human subject, approximately 3.5×10⁵ or more Tmem per kilogram of body weight of the human subject, approximately 4.0×10⁵ or more Tmem per kilogram of body weight of the human subject, approximately 4.5×10⁵ or more Tmem per kilogram of body weight of the human subject, approximately 5.0×10⁵ or more Tmem per kilogram of body weight of the human subject, approximately 5.5×10⁵ or more Tmem per kilogram of body weight of the human subject, approximately 6.0×10⁵ or more Tmem per kilogram of body weight of the human subject, approximately 6.5×10⁵ or more Tmem per kilogram of body weight of the human subject, approximately 7.0×10⁵ or more Tmem per kilogram of body weight of the human subject, approximately 7.5×10⁵ or more Tmem per kilogram of body weight of the human subject, approximately 8.0×10⁵ or more Tmem per kilogram of body weight of the human subject, approximately 8.5×10⁵ or more Tmem per kilogram of body weight of the human subject, approximately 9.0×10⁵ or more Tmem per kilogram of body weight of the human subject, approximately 9.5×10⁵ or more Tmem per kilogram of body weight of the human subject, approximately 1.0×10⁶ or more Tmem per kilogram of body weight of the human subject, approximately 1.25×10⁶ or more Tmem per kilogram of body weight of the human subject, approximately 1.5×10⁶ or more Tmem per kilogram of body weight of the human subject, approximately 1.75×10⁶ or more Tmem per kilogram of body weight of the human subject, approximately 2.0×10⁶ or more Tmem per kilogram of body weight of the human subject, approximately 2.25×10⁶ or more Tmem per kilogram of body weight of the human subject, approximately 2.5×10⁶ or more Tmem per kilogram of body weight of the human subject, approximately 2.75×10⁶ or more Tmem per kilogram of body weight of the human subject, approximately 3.0×10⁶ or more Tmem per kilogram of body weight of the human subject, approximately 3.25×10⁶ or more Tmem per kilogram of body weight of the human subject, approximately 3.5×10⁶ or more Tmem per kilogram of body weight of the human subject, approximately 3.75×10⁶ or more Tmem per kilogram of body weight of the human subject, approximately 4.0×10⁶ or more Tmem per kilogram of body weight of the human subject, approximately 4.25×10⁶ or more Tmem per kilogram of body weight of the human subject, approximately 4.5×10⁶ or more Tmem per kilogram of body weight of the human subject, approximately 4.75×10⁶ or more Tmem per kilogram of body weight of the human subject, approximately 5.0×10⁶ or more Tmem per kilogram of body weight of the human subject, approximately 5.25×10⁶ or more Tmem per kilogram of body weight of the human subject, approximately 5.5×10⁶ or more Tmem per kilogram of body weight of the human subject, approximately 5.75×10⁶ or more Tmem per kilogram of body weight of the human subject, approximately 6.0×10⁶ or more Tmem per kilogram of body weight of the human subject, approximately 6.25×10⁶ or more Tmem per kilogram of body weight of the human subject, approximately 6.5×10⁶ or more Tmem per kilogram of body weight of the human subject, approximately 6.75×10⁶ or more Tmem per kilogram of body weight of the human subject, approximately 7.0×10⁶ or more Tmem per kilogram of body weight of the human subject, approximately 7.25×10⁶ or more Tmem per kilogram of body weight of the human subject, approximately 7.5×10⁶ or more Tmem per kilogram of body weight of the human subject, approximately 7.75×10⁶ or more Tmem per kilogram of body weight of the human subject, approximately 8.0×10⁶ or more Tmem per kilogram of body weight of the human subject, approximately 8.25×10⁶ or more Tmem per kilogram of body weight of the human subject, approximately 8.5×10⁶ or more Tmem per kilogram of body weight of the human subject, approximately 8.75×10⁶ or more Tmem per kilogram of body weight of the human subject, approximately 9.0×10⁶ or more Tmem per kilogram of body weight of the human subject, approximately 9.25×10⁶ or more Tmem per kilogram of body weight of the human subject, approximately 9.5×10⁶ or more Tmem per kilogram of body weight of the human subject, approximately 9.75×10⁶ or more Tmem per kilogram of body weight of the human subject, approximately 1.0×10⁷ or more Tmem per kilogram of body weight of the human subject, approximately 1.25×10⁷ or more Tmem per kilogram of body weight of the human subject, approximately 1.5×10⁷ or more Tmem per kilogram of body weight of the human subject, approximately 1.75×10⁷ or more Tmem per kilogram of body weight of the human subject, approximately 2.0×10⁷ or more Tmem per kilogram of body weight of the human subject, approximately 2.25×10⁷ or more Tmem per kilogram of body weight of the human subject, approximately 2.5×10⁷ or more Tmem per kilogram of body weight of the human subject, approximately 2.75×10⁷ or more Tmem per kilogram of body weight of the human subject, approximately 3.0×10⁷ or more Tmem per kilogram of body weight of the human subject, approximately 3.25×10⁷ or more Tmem per kilogram of body weight of the human subject, approximately 3.5×10⁷ or more Tmem per kilogram of body weight of the human subject, approximately 3.75×10⁷ or more Tmem per kilogram of body weight of the human subject, approximately 4.0×10⁷ or more Tmem per kilogram of body weight of the human subject, approximately 4.25×10⁷ or more Tmem per kilogram of body weight of the human subject, approximately 4.5×10⁷ or more Tmem per kilogram of body weight of the human subject, approximately 4.75×10⁷ or more Tmem per kilogram of body weight of the human subject, approximately 5.0×10⁷ or more Tmem per kilogram of body weight of the human subject, approximately 5.25×10⁷ or more Tmem per kilogram of body weight of the human subject, approximately 5.5×10⁷ or more Tmem per kilogram of body weight of the human subject, approximately 5.75×10⁷ or more Tmem per kilogram of body weight of the human subject, approximately 6.0×10⁷ or more Tmem per kilogram of body weight of the human subject, approximately 6.25×10⁷ or more Tmem per kilogram of body weight of the human subject, approximately 6.5×10⁷ or more Tmem per kilogram of body weight of the human subject, approximately 6.75×10⁷ or more Tmem per kilogram of body weight of the human subject, approximately 7.0×10⁷ or more Tmem per kilogram of body weight of the human subject, approximately 7.25×10⁷ or more Tmem per kilogram of body weight of the human subject, approximately 7.5×10⁷ or more Tmem per kilogram of body weight of the human subject, approximately 7.75×10⁷ or more Tmem per kilogram of body weight of the human subject, approximately 8.0×10⁷ or more Tmem per kilogram of body weight of the human subject, approximately 8.25×10⁷ or more Tmem per kilogram of body weight of the human subject, approximately 8.5×10⁷ or more Tmem per kilogram of body weight of the human subject, approximately 8.75×10⁷ or more Tmem per kilogram of body weight of the human subject, approximately 9.0×10⁷ or more Tmem per kilogram of body weight of the human subject, approximately 9.25×10⁷ or more Tmem per kilogram of body weight of the human subject, approximately 9.5×10⁷ or more Tmem per kilogram of body weight of the human subject, approximately 9.75×10⁷ or more Tmem per kilogram of body weight of the human subject, approximately 1.0×10⁸ or more Tmem per kilogram of body weight of the human subject, approximately 1.25×10⁸ or more Tmem per kilogram of body weight of the human subject, approximately 1.5×10⁸ or more Tmem per kilogram of body weight of the human subject, approximately 1.75×10⁸ or more Tmem per kilogram of body weight of the human subject, approximately 2.0×10⁸ or more Tmem per kilogram of body weight of the human subject, approximately 2.25×10⁸ or more Tmem per kilogram of body weight of the human subject, approximately 2.5×10⁸ or more Tmem per kilogram of body weight of the human subject, approximately 2.75×10⁸ or more Tmem per kilogram of body weight of the human subject, approximately 3.0×10⁸ or more Tmem per kilogram of body weight of the human subject, approximately 3.25×10⁸ or more Tmem per kilogram of body weight of the human subject, approximately 3.5×10⁸ or more Tmem per kilogram of body weight of the human subject, approximately 3.75×10⁸ or more Tmem per kilogram of body weight of the human subject, approximately 4.0×10⁸ or more Tmem per kilogram of body weight of the human subject, approximately 4.25×10⁸ or more Tmem per kilogram of body weight of the human subject, approximately 4.5×10⁸ or more Tmem per kilogram of body weight of the human subject, approximately 4.75×10⁸ or more Tmem per kilogram of body weight of the human subject, or approximately 5.0×10⁸ or more Tmem per kilogram of body weight of the human subject. In some embodiments, the body weight of the human subject is actual body weight of the human subject. In other embodiments, the body weight of the human subject is ideal actual body weight of the human subject.

In some embodiments, a cell population of the present disclosure, for example the first population of CD45⁺ cells, the second population of CD45⁺ cells, and/or the third population of CD45⁺ cells, may comprise one or more cells comprising one or more chimeric receptors. In some embodiments, the one or more cells may be T cells, NK cells, CTLs, Tregs, and/or NKT cells. In some embodiments, the one or more chimeric receptors comprise one or more extracellular antigen-binding domains that bind to one or more cancer-associated antigens. Any suitable cancer-associated antigen known in the art may be targeted. In some embodiments, the chimeric receptor is a chimeric antigen receptor or a T cell receptor.

Provided herein are compositions, multi-component pharmaceutical treatments, multi-component cellular therapy products, cell populations, solutions, formulations, kits, and/or methods for improved hematopoietic stem cell transplantation (HCT), for example, allogeneic hematopoietic stem cell transplantation (alloHSCT). Compositions, multi-component pharmaceutical treatments, multi-component cellular therapy products, cell populations, solutions, formulations, kits, and/or methods disclosed herein can comprise one or more cell populations that can be administered in combination with a GVHD prophylactic agent to achieve positive clinical outcomes. A cell population can comprise one or more types of cells, for example, hematopoietic stem and progenitor cells (HSPCs), conventional T cells (Tcons), regulatory T cells (Tregs), invariant natural killer T cells (iNKTs), memory T cells (Tmems), and combinations thereof.

The disclosure provides parameters for cell populations and methods of administering cell populations that can contribute to successful clinical outcomes in HCT recipient subjects. Without wishing to be bound by theory, parameters that can contribute to successful clinical outcomes in HCT recipient subjects include, for example, co-administration of a GVHD prophylactic agent as described herein (e.g., tacrolimus), populations administered, order and timing for the administration of different populations, purity standards for populations, methods for obtaining populations, methods of handling or storing populations, dosages of populations administered, methods for obtaining populations, and combinations thereof.

In various embodiments, administering comprises infusing into the human subject the first population of CD45⁺ cells, the population of cells enriched for Tregs (e.g., the second population of CD45⁺ cells), and the third population of CD45⁺ cells.

In some embodiments, the population of cells enriched for Tregs or the second population of CD45⁺ cells (as disclosed herein) is administered from approximately 5 minutes to approximately 5 hours after administration of the first population of CD45⁺ cells (as disclosed herein). In some embodiments, the population of cells enriched for Tregs or the second population of CD45⁺ cells is administered approximately 5 minutes, approximately 6 minutes, approximately 7 minutes, approximately 8 minutes, approximately 9 minutes, approximately 10 minutes, approximately 11 minutes, approximately 12 minutes, approximately 13 minutes, approximately 14 minutes, approximately 15 minutes, approximately 16 minutes, approximately 17 minutes, approximately 18 minutes, approximately 19 minutes, approximately 20 minutes, approximately 21 minutes, approximately 22 minutes, approximately 23 minutes, approximately 24 minutes, approximately 25 minutes, approximately 26 minutes, approximately 27 minutes, approximately 28 minutes, approximately 29 minutes, approximately 30 minutes, approximately 31 minutes, approximately 32 minutes, approximately 33 minutes, approximately 34 minutes, approximately 35 minutes, approximately 36 minutes, approximately 37 minutes, approximately 38 minutes, approximately 39 minutes, approximately 40 minutes, approximately 41 minutes, approximately 42 minutes, approximately 43 minutes, approximately 44 minutes, approximately 45 minutes, approximately 46 minutes, approximately 47 minutes, approximately 48 minutes, approximately 49 minutes, approximately 50 minutes, approximately 51 minutes, approximately 52 minutes, approximately 53 minutes, approximately 54 minutes, approximately 55 minutes, approximately 56 minutes, approximately 57 minutes, approximately 58 minutes, approximately 59 minutes, approximately 1 hour, approximately 1.10 hours, approximately 1.20 hours, approximately 1.30 hours, approximately 1.40 hours, approximately 1.50 hours, approximately 1.60 hours, approximately 1.70 hours, approximately 1.80 hours, approximately 1.90 hours, approximately 2 hours, approximately 2.10 hours, approximately 2.20 hours, approximately 2.30 hours, approximately 2.40 hours, approximately 2.50 hours, approximately 2.60 hours, approximately 2.70 hours, approximately 2.80 hours, approximately 2.90 hours, approximately 3 hours, approximately 3.10 hours, approximately 3.20 hours, approximately 3.30 hours, approximately 3.40 hours, approximately 3.50 hours, approximately 3.60 hours, approximately 3.70 hours, approximately 3.80 hours, approximately 3.90 hours, approximately 4 hours, approximately 4.10 hours, approximately 4.20 hours, approximately 4.30 hours, approximately 4.40 hours, approximately 4.50 hours, approximately 4.60 hours, approximately 4.70 hours, approximately 4.80 hours, approximately 4.90 hours, or approximately 5 hours after administration of the first population of CD45⁺ cells.

In some embodiments, the third population of CD45⁺ cells (as disclosed herein) is administered at least about 12 hours after the first population of CD45⁺ cells (as disclosed herein), the third population of CD45⁺ cells is administered from approximately 24 to approximately 96 hours after the first population of CD45⁺ cells, the third population of CD45⁺ cells is administered from approximately 36 to approximately 60 hours after the first population of CD45⁺ cells, the third population of CD45⁺ cells is administered at least about 12 hours after the population of cells enriched for Tregs (as disclosed herein), the third population of CD45⁺ cells is administered from approximately 24 to approximately 96 hours after the population of cells enriched for Tregs, and/or the third population of CD45⁺ cells is administered from approximately 36 to approximately 60 hours after the population of cells enriched for Tregs or the second population of CD45⁺ cells.

In some embodiments, the third population of CD45⁺ cells (as disclosed herein) is administered from approximately 12 hours to approximately 120 hours after administration of the first population of isolated CD45⁺ cells (as disclosed herein). In some embodiments, the third population of CD45⁺ cells is administered approximately 12 hours, approximately 13 hours, approximately 14 hours, approximately 15 hours, approximately 16 hours, approximately 17 hours, approximately 18 hours, approximately 19 hours, approximately 20 hours, approximately 21 hours, approximately 22 hours, approximately 23 hours, approximately, 24 hours, approximately 25 hours, approximately 26 hours, approximately 27 hours, approximately 28 hours, approximately 29 hours, approximately 30 hours, approximately 31 hours, approximately 32 hours, approximately 33 hours, approximately, 34 hours, approximately 35 hours, approximately 36 hours, approximately 37 hours, approximately 38 hours, approximately 39 hours, approximately 40 hours, approximately 41 hours, approximately 42 hours, approximately 43 hours, approximately, 44 hours, approximately 45 hours, approximately 46 hours, approximately 47 hours, approximately 48 hours, approximately 49 hours, approximately 50 hours, approximately 51 hours, approximately 52 hours, approximately 53 hours, approximately, 54 hours, approximately 55 hours, approximately 56 hours, approximately 57 hours, approximately 58 hours, approximately 59 hours, approximately 60 hours, approximately 61 hours, approximately 62 hours, approximately 63 hours, approximately, 64 hours, approximately 65 hours, approximately 66 hours, approximately 67 hours, approximately 68 hours, approximately 69 hours, approximately 70 hours, approximately 71 hours, approximately 72 hours, approximately 73 hours, approximately, 74 hours, approximately 75 hours, approximately 76 hours, approximately 77 hours, approximately 78 hours, approximately 79 hours, approximately 80 hours, approximately 81 hours, approximately 82 hours, approximately 83 hours, approximately, 84 hours, approximately 85 hours, approximately 86 hours, approximately 87 hours, approximately 88 hours, approximately 89 hours, approximately 90 hours, approximately 91 hours, approximately 92 hours, approximately 93 hours, approximately, 94 hours, approximately 95 hours, approximately 96 hours, approximately 97 hours, approximately 98 hours, approximately 99 hours, approximately 100 hours, approximately 101 hours, approximately 102 hours, approximately 103 hours, approximately, 104 hours, approximately 105 hours, approximately 106 hours, approximately 107 hours, approximately 108 hours, approximately 109 hours, approximately 110 hours, approximately 111 hours, approximately 112 hours, approximately 113 hours, approximately, 114 hours, approximately 115 hours, approximately 116 hours, approximately 117 hours, approximately 118 hours, approximately 119 hours, or approximately 120 hours after administration of the first population of CD45⁺ cells.

In some embodiments, the third population of CD45⁺ cells (as disclosed herein) is administered from approximately 12 hours to approximately 120 hours after administration of the population of cells enriched for Tregs or the second population of CD45⁺ cells (as disclosed herein). In some embodiments, the third population of CD45⁺ cells is administered approximately 12 hours, approximately 13 hours, approximately 14 hours, approximately 15 hours, approximately 16 hours, approximately 17 hours, approximately 18 hours, approximately 19 hours, approximately 20 hours, approximately 21 hours, approximately 22 hours, approximately 23 hours, approximately, 24 hours, approximately 25 hours, approximately 26 hours, approximately 27 hours, approximately 28 hours, approximately 29 hours, approximately 30 hours, approximately 31 hours, approximately 32 hours, approximately 33 hours, approximately, 34 hours, approximately 35 hours, approximately 36 hours, approximately 37 hours, approximately 38 hours, approximately 39 hours, approximately 40 hours, approximately 41 hours, approximately 42 hours, approximately 43 hours, approximately, 44 hours, approximately 45 hours, approximately 46 hours, approximately 47 hours, approximately 48 hours, approximately 49 hours, approximately 50 hours, approximately 51 hours, approximately 52 hours, approximately 53 hours, approximately, 54 hours, approximately 55 hours, approximately 56 hours, approximately 57 hours, approximately 58 hours, approximately 59 hours, approximately 60 hours, approximately 61 hours, approximately 62 hours, approximately 63 hours, approximately, 64 hours, approximately 65 hours, approximately 66 hours, approximately 67 hours, approximately 68 hours, approximately 69 hours, approximately 70 hours, approximately 71 hours, approximately 72 hours, approximately 73 hours, approximately, 74 hours, approximately 75 hours, approximately 76 hours, approximately 77 hours, approximately 78 hours, approximately 79 hours, approximately 80 hours, approximately 81 hours, approximately 82 hours, approximately 83 hours, approximately, 84 hours, approximately 85 hours, approximately 86 hours, approximately 87 hours, approximately 88 hours, approximately 89 hours, approximately 90 hours, approximately 91 hours, approximately 92 hours, approximately 93 hours, approximately, 94 hours, approximately 95 hours, approximately 96 hours, approximately 97 hours, approximately 98 hours, approximately 99 hours, approximately 100 hours, approximately 101 hours, approximately 102 hours, approximately 103 hours, approximately, 104 hours, approximately 105 hours, approximately 106 hours, approximately 107 hours, approximately 108 hours, approximately 109 hours, approximately 110 hours, approximately 111 hours, approximately 112 hours, approximately 113 hours, approximately, 114 hours, approximately 115 hours, approximately 116 hours, approximately 117 hours, approximately 118 hours, approximately 119 hours, or approximately 120 hours after administration of the population of cells enriched for Tregs or the second population of CD45⁺ cells.

HSPCs can have extensive self-renewal capacity, and an ability to differentiate into specialized cell types, for example, an ability to reconstitute all hematopoietic cell lineages. HSPCs can undergo asynchronous replication, where two daughter cells are produced with different phenotypes. HSPCs cells can exist in a mitotically quiescent form. HSPCs can be derived from bone marrow, peripheral blood, and/or umbilical cord blood.

Subsets of immune cells, such as conventional T cells (Tcons), regulatory T cells (Tregs), invariant natural killer T cells (iNKTs), and memory T cells (Tmems) can contribute to aspects of GVHD following HCT, and can also contribute to, for example, GVT immune responses, immune reconstitution, infection susceptibility, and patient survival.

GVHD can be mediated in large part by donor T cells, which can elicit inflammatory responses upon recognition of recipient antigens. T cell depletion (TCD) of cell populations for transplantation to a subject can be undertaken to decrease the likelihood of acute and/or chronic GVHD. T cells can be depleted using methods including, but not limited to, physical adsorption of T cells to protein ligands such as lectins, immunodepletion with T cell specific antibodies, and immunoaffinity techniques (for example, use of T cell or lymphocyte-specific antibodies in immunoadsorption columns, magnetic activated cell sorting (MACS), or fluorescent activated cell sorting (FACS)). Applying TCD techniques to donor grafts can result in, for example, 10-fold to 10⁵-fold depletion of T cells, and reduced incidence of GVHD. However, TCD can also result in increased incidence of cancer relapse, as the lack of T cells can reduce a graft-versus-tumor (GVT) immune response. Additionally, TCD can result in impaired immune recovery, and increased susceptibility to infections.

Both GVT and GVHD can be largely mediated by conventional T cells (Tcons), which mount immune responses upon recognition of cognate antigen by T cell receptors (tumor antigens for GVT, non-tumor recipient antigens for GVHD). Tcons can, for example, contribute to GVT, GVHD, or a combination thereof. In some embodiments, administration of Tcons after administration of Tregs can enhance GVT immunity, and/or reduce susceptibility to infection.

Tcons can broadly refer to all CD3⁺ T cells, cells expressing CD3 and CD4 or cells expressing CD3 and CD8, cells expressing medium to high levels of CD127, cells expressing CD3 and medium to high levels of CD127, cells expressing CD3, cells expressing medium to high levels of CD127, and cells expressing CD4 or CD8. In some embodiments, Tcons do not express Vα24Jα18 TCR. Tcons and Regulatory T cells (“Tregs”) can be non-mutually-exclusive cell populations. In some embodiments, Tcons and Tregs are mutually exclusive cell populations.

Regulatory T cells (“Tregs”) are a specialized subpopulation of T cells that negatively regulate (e.g., suppress) activation of the immune system and thereby promote immune tolerance. Without wishing to be bound by theory, cell populations of the disclosure enriched for Tregs contribute to positive clinical outcomes by, for example, reducing the incidence and/or severity of GVHD in a transplant recipient subject, and/or improving immune reconstitution in a transplant recipient. Administering cell population enriched for Tregs with a population of CD45⁺ cells that comprises, at least, HSPCs can, for example, facilitate retention of graft versus tumor (GVT) and reduced incidence and/or severity of GVHD. Without wishing to be bound by theory, administering population of cells enriched for Tregs can prevent GVHD, and administering third population of CD45⁺ cells that comprises, at least, Tcons can promote GVT effects, for example, relative to alternate hematopoietic stem cell transplantation (HCT) methods, i.e., methods that are distinct from the compositions, multi-component pharmaceutical treatments, multi-component cellular therapy products, cell populations, solutions, formulations, kits, and/or methods disclosed herein. In some embodiments, administering a population of cells enriched for Tregs reduces the risk of developing GVHD, and administering third population of CD45⁺ cells that comprises, at least, Tcons promotes GVT effects relative to alternate HCT methods, i.e., methods that are distinct from the compositions, multi-component pharmaceutical treatments, multi-component cellular therapy products, cell populations, solutions, formulations, kits, and/or methods disclosed herein.

As used herein, an alternate composition lacks one or more cell populations and/or prophylactic agent that are disclosed herein. As examples, an alternate composition lacks one or more of a cell population comprising HSPCs, a cell population comprising Tregs, a cell population comprising Tcons, and a prophylactic agent. In some embodiments, an alternate composition or treatment regimen comprises an additional cell population or agent compared to a composition or treatment regimen of the disclosure, e.g., a an additional or different GVHD prophylactic agent.

There are a number of subsets of Tregs, for example, TCRαβ⁺CD4⁺ regulatory T cells, which include natural regulatory T cells (nTregs) and induced regulatory T cells (iTregs). nTregs can be T cells produced in the thymus and delivered to the periphery as a long-lived lineage of self-antigen-specific lymphocytes. iTregs can be recruited from circulating lymphocytes and acquire regulatory properties under particular conditions of stimulation in the periphery. nTregs and iTregs are CD4⁺CD25⁺; both can inhibit proliferation of CD4⁺CD25− T cells in a dose-dependent manner. In some embodiments, Tregs are anergic and do not proliferate upon TCR stimulation. In addition to being positive for CD4 and CD25, Tregs can be positive for the transcription factor FOXP3, an intracellular marker. Tregs can be identified or selected based on various marker expression profiles. Non-limiting examples of marker expression profiles that can be used to select Tregs include (1) CD4⁺CD25⁺CD127^{dim}, (2) CD4⁺FOXP3⁺, (3) CD3⁺CD4⁺CD25⁺, (4) CD3⁺CD4⁺CD25⁺CD127^{dim}, (5) CD3⁺CD4⁺CD25⁺CD127^{dim}FOXP3⁺, (6) CD3⁺FOXP3⁺, (7) CD3⁺CD4⁺FOXP3⁺, (8) CD3⁺ CD4⁺CD25⁺FOXP3⁺, (9) CD3⁺CD25⁺FOXP3⁺, (10) CD3⁺CD25⁺CD127^{dim}, (11) CD4⁺CD25⁺, (12) CD4⁺CD25⁺CD127^{dim}FOXP3⁺, (13) FOXP3⁺, (14) CD4⁺FOXP3⁺, (15) CD4⁺CD25⁺FOXP3⁺, (16) CD25⁺FOXP3⁺, and (17) CD25⁺CD127^{dim}.

Selection based on certain expression profiles can be achieved based on extracellular markers and without requiring cell permeabilization, for example, selection based on CD4⁺CD25⁺CD127^{dim}.

A cell population that comprises Tregs can, for example, reduce the incidence of graft rejection, reduce the incidence and/or severity of GVHD, promote hematopoietic reconstitution, promote immune reconstitution, promote mixed chimerism, or a combination thereof.

A cell population of the disclosure can comprise invariant natural killer T cells (INKTs). iNKTs are subclass of CD1d-restricted Natural Killer T (NKT) cells that express a highly conserved αβ-T cell receptor that comprises of Vα24Jα18 TCRα chain in humans (referred to herein as “Vα24Jα18⁺”). iNKT cells can be identified by binding with CD1d-multimers like that are loaded with α-galactosylceramide (GalCer), PBS-57, PBS-44 or other natural or synthetic glycolipids. Another method of identification is an antibody or combination of antibodies that specifically recognize the Vα24Jα18 region. Examples include a Vα24 antibody, a Jα18 antibody, or the monoclonal antibody clone 6B11 which binds specifically to a unique region of the Vα24Jα18 TCR and can be used to identify iNKT cells. iNKTs can be CD3⁺Vα24Jα18⁺.

In some embodiments, iNKTs can promote engraftment, promote GVT, reduce incidence and/or severity of GVHD, decrease susceptibility to cancer relapse, decrease susceptibility to infection, or a combination thereof. In some embodiments, iNKTs promote the activity of Tregs. In some embodiments, iNKTs promote the activity of HSPCs.

A cell population of the disclosure can comprise memory T cells (Tmems). Tmems can refer to antigen-experienced T cells that express, for example, the phenotypic markers CD45RO, TCRα, TCRβ, CD3, CD4, CD95, and IL-2Rβ or the phenotypic markers CD45RO, TCRα, TCRβ, CD3, CD8, CD95, and IL-2Rβ. Tmems provide immunity and are capable of persisting for a long period of time in an inactive state. Tmems are able to rapidly acquire effector functions upon re-challenge with antigen. A population of Tmems can include any combination of the subclasses T central memory cells and T effector memory cells. In some embodiments, Tmems are CD3⁺CD45RA⁻CD45RO⁺. In various methods, Tmems administered to a subject receiving HCT can, for example, promote GVT, reduce GVHD, decrease susceptibility to cancer relapse, decrease susceptibility to infection, or a combination thereof.

### Acquisition, Processing, and Preparation of Cells

Certain aspects of the present disclosure relate to methods for preparing the compositions, multi-component pharmaceutical treatments, multi-component cellular therapy products, cell populations, solutions, formulations, and/or kits of the present disclosure.

In some embodiments, at least one mobilized peripheral blood donation is collected from a donor or at most two mobilized peripheral blood donations are collected from the donor. In some embodiments, the mobilized peripheral blood donation is an HSPC-mobilized peripheral blood apheresis donation. In some embodiments, prior to peripheral blood donation, the donor may be vaccinated with a tumor antigen and/or a pathogen to enhance graft versus infection.

In embodiments, at least one of the mobilized peripheral blood donations is processed and sorted to enrich CD34⁺ cells and Tregs. In some embodiments, the peripheral blood donation is further processed and sorted or alternatively processed and sorted to enrich CD3⁺ cells (e.g., Tcons). In embodiments, at least one of the mobilized peripheral blood donations is processed and sorted to enrich CD34⁺ cells, Tregs, and/or Tcons. The processing and sorting for the CD34⁺ cells, Tregs, and Tcons maybe done in any odder. For example, at least one of the mobilized peripheral blood donations may be processed and sorted to first enrich CD34⁺ cells, then Tcons, and then Tregs. Alternatively, at least one of the mobilized peripheral blood donations may be processed and sorted to first enrich CD34⁺ cells, then Tregs, and then Tcons. Alternatively, at least one of the mobilized peripheral blood donations may be processed and sorted to first enrich Tregs, then CD34⁺ cells, and then Tcons. Alternatively, at least one of the mobilized peripheral blood donations may be processed and sorted to first enrich Tregs, then Tcons, and then CD34⁺ cells. Alternatively, at least one of the mobilized peripheral blood donations may be processed and sorted to first enrich Tcons, then CD34⁺ cells, and then Tregs. Alternatively, at least one of the mobilized peripheral blood donations may be processed and sorted to first enrich Tcons, then Tregs, and then CD34⁺ cells. In some embodiments, at least one of the mobilized peripheral blood donations may be processed and sorted to first enrich Tregs and then CD34⁺ cells, without enrichment for Tcons. In other embodiments, at least one of the mobilized peripheral blood donations may be processed and sorted to first enrich CD34⁺ cells and then Tregs without enrichment for Tcons.

In some embodiments, the processing and sorting time of the one or more of the mobilized peripheral blood donations is less than about 40 hours, the processing and sorting time of the one or more of the mobilized peripheral blood donations is less than about 35 hours, the processing and sorting time of the one or more of the mobilized peripheral blood donations is less than about 30 hours, the processing and sorting time of the one or more of the mobilized peripheral blood donations is less than about 25 hours, the processing and sorting time of the one or more of the mobilized peripheral blood donations is less than about 20 hours, the processing and sorting time of the one or more of the mobilized peripheral blood donations is less than about 15 hours, and/or the processing and sorting time of the one or more of the mobilized peripheral blood donations is at most about 35 hours, the processing and sorting time of the one or more of the mobilized peripheral blood donations is at most about 30 hours, the processing and sorting time of the one or more of the mobilized peripheral blood donations is at most about 25 hours, the processing and sorting time of the one or more of the mobilized peripheral blood donations is at most about 20 hours, or the processing and sorting time of the one or more of the mobilized peripheral blood donations is at most about 15 hours.

In various embodiments, the one or more of the mobilized peripheral blood donations is processed and sorted using one or more immune-separation particles (ISPs), e.g., ISPs comprise affinity reagents such as immuno-magnetic separation particles which may be antibodies each conjugated to an iron-containing particle, and/or immuno-fluorescent separation particles which may be antibodies each conjugated to a fluorophore or other fluorescent particle.

In some embodiments, a cell population of the disclosure is obtained from whole blood. A cell population of the disclosure can be obtained from a peripheral blood apheresis product, for example, a mobilized peripheral blood apheresis product, e.g., mobilized by administration of GCSF, GM-CSF, MOZOBIL® (plerixafor), and combinations thereof, to a donor. A cell population of the disclosure can be obtained from at least one apheresis product, two apheresis products, three apheresis products, four apheresis products, five apheresis products, six apheresis products, or more. In some embodiments, a cell population of the disclosure is obtained from one apheresis product. In some embodiments, a cell population of the disclosure is obtained from two apheresis products. In some embodiments, a cell population of the disclosure is obtained from an apheresis product from one donor and an apheresis product from an at least second donor.

In some embodiments, a cell population of the disclosure is obtained from bone marrow sample. In some embodiments, a cell population of the disclosure can be obtained from a bone marrow ample, for example, a mobilized bone marrow sample, e.g., mobilized by administration of GCSF, GM-CSF, MOZOBIL® (plerixafor), and combinations thereof, to a donor.

In some embodiments, a cell population of the disclosure is obtained from umbilical cord blood.

A cell population of the disclosure can be refined by selection from a population of cells, for example, peripheral blood or a peripheral blood apheresis product. Selection methods for cell populations can comprise methods involving positive or negative selection of a cell population of interest. Selection methods for cell populations can comprise affinity reagents, including but not limited to an antibody, a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a full-length DARPin, a fragment of a DARPin, a full-length fynomer, a fragment of a fynomer, a full-length kunitz domain peptide, a fragment of a kunitz domain peptide, a full-length monobody, a fragment of a monobody, a peptide, or a polyaminoacid. In some embodiments, the affinity reagent is directly conjugated to a detection reagent and/or purification reagent. In some cases, the detection reagent and purification reagent are the same. In some cases, the detection reagent and purification reagent are different. For example, the detection reagent and/or purification reagent is fluorescent, magnetic, or the like. In some cases, the detection reagent and/or purification reagent is a magnetic particle for column purification. For example, magnetic column purification may be performed using the Miltenyi system (CliniMACs) of columns, antibodies, buffers, preparation materials and reagents.

In various embodiments, at least one of the cell populations have a plurality of immuno-separation particles (ISPs) attached to receptors on the cells of the cell population. In some cases, the plurality of ISPs are immuno-magnetic separation particles. In some embodiments, the plurality of ISPs comprise an antibody conjugated to an iron containing particle. In some cases, at least a portion of the plurality of ISPs are attached to CD34⁺ receptors on the HPSC's of the HSPC cell population; optionally, an average number of ISPs per HSPC in the HSPC cell population is less than about 6,000, an average number of ISPs per HSPC in the HSPC cell population is equal to or less than about 3,000, and/or an average number of ISPs per HSPC in the HSPC cell population is from approximately 1700 to approximately 3,000. In some cases, at least a portion of the plurality of ISPs are attached to CD25⁺ receptors on the cells of the Treg cell population; optionally, an average number of ISPs per T-reg cell in the Treg population is equal or less than about 1700 or an average number of ISPs per T-reg cell in the Treg population is from approximately 1400 to approximately 1700. In some cases, at least a portion of the plurality of ISPs are attached to CD3⁺ receptors on the cells of the heterogenous cell population; optionally, an average number of ISPs per cell in population of T heterogenous is less than about 1,000.

Affinity reagents can comprise immunoaffinity reagents, utilizing the binding specificity of antibodies or fragments or derivatives thereof to positively or negatively select for a cell population of interest. Selection methods for cell populations can comprise an affinity agent and a column, such as magnetic activated cell sorting (MACS) with specific antibodies and microbeads. Selection methods for cell populations can comprise fluorescent activated cell sorting (FACS), with cell populations sorted based on staining profiles with one or more fluorescently-conjugated antibodies. Selection methods for cell populations can comprise physical adsorption, for example, physical adsorption of T cells to protein ligands such as lectins.

HSPCs can be obtained by harvesting from bone marrow or from peripheral blood. Bone marrow can be aspirated from the posterior iliac crest or the anterior iliac crest while the donor is under either local or general anesthesia. HSPCs can be obtained by harvesting from peripheral blood, for example, by peripheral blood apheresis. The number of stem cells harvested can be increased by treating the donor with a mobilization agent, i.e., an agent that mobilizes stem cells from the bone marrow into peripheral blood. Non-limiting examples of mobilization agents include granulocyte colony-stimulating factor (G-CSF), granulocyte macrophage colony-stimulating factor (GM-CSF), stem cell factor (SCF), a SDF-1 antagonist, a CXCR4 antagonist (e.g., POL6326, BKT-140, TG-0054, NOX-A12), MOZOBIL® (plerixafor), a CXCR2 ligand (e.g., GROβ), a sphingosine-1-phosphatase (S1P) agonist. (e.g., SEW2871), a VCAM/VLA-4 inhibitor (e.g., BIO5192), a proteosome inhibitor (e.g., Bortezomib), parathyroid hormone, a hypoxia inducible factor (HIF) stabilizer (e.g., FG-4497), and combinations thereof. Techniques to mobilize stem cells into peripheral blood can comprise administering to a donor, for example, 10 to 40 μ/kg/day of a mobilization agent. A mobilization agent can be administered to the donor in, for example, 1, 2, 3, 4, 5, 6, 7, 8, 9, or 10 doses. An apheresis product can be isolated from a donor about, for example, 1, 2, 3, 4, 5, 6, 7, 8, 9, 10, 11, 12, 13, 14, 15, 16, 17, 18, 19, 20, 21, 22, 23, 24, 26, 28, or 30 hour(s) after a dose of mobilization agent.

A population of CD45⁺ cells of the disclosure can comprise a HSPCs. The HSPCs can be selected based on expression of CD34. For example, the HSPCs of the disclosure can be selected using anti-CD34 antibodies as part of a magnetic activated cell sorting (MACS) or fluorescent activated cell sorting (FACS) system.

The number of HPSCs in a population of CD45⁺ cells can be determined, for example, by quantifying CD34⁺ cells via flow cytometry. In some embodiments, dose calculations are adjusted based on measures of cell viability measurements, e.g., viability determined via flow cytometry with propidium iodide or 7-AAD, or via trypan blue exclusion.

A cell population of the disclosure can be enriched for Tregs (e.g., the second population of CD45⁺ cells). Tregs can be selected based on expression of markers including CD3, CD4, CD25, CD127, FOXP3, and combinations thereof.

Tregs can be selected using magnetic activated cell sorting (MACS). Tregs can be selected using fluorescent activated cell sorting (FACS). Tregs can be selected using multiple procedures, for example, multiple MACS selections, multiple FACS selections, or a combination of MACS and FACS selections. For example, a first selection may be performed for expression of CD25, isolating CD25⁺ cells from a hematopoietic cell sample, for example with MACS. A second selection may be performed by contacting the CD25⁺ cells with antibodies specific for CD4 and for CD127, where FACS is used to isolate cells that are CD4⁺CD127^{dim}.

Tregs can be isolated from whole blood. Tregs can be isolated from a peripheral blood apheresis product. Tregs can be isolated from a population of cells previously enriched and/or depleted for one or more other cell types, e.g., isolated from a population of cells depleted of CD34⁺ cells. In some embodiments, Tregs are isolated from the flow-through fraction of a CD34⁺ MACS selection.

The number of Tregs in a population of cells can be determined, for example, by flow cytometry, where Tregs can be identified as, for example, CD4⁺CD25⁺CD127^{dim} or CD4⁺FOXP3⁺. Dose calculations can be adjusted based on measures of cell viability measurements, e.g., viability determined via flow cytometry with propidium iodide or 7-AAD, or via trypan blue exclusion.

In some embodiments, the third population of CD45⁺ cells can comprise a population of Tcons. The third population of CD45⁺ cells that comprises, at least, Tcons can be sourced from peripheral blood. The third population of CD45⁺ cells can be sourced from a peripheral blood apheresis product.

In some embodiments, no selection steps are carried out, and a population of CD45⁺ cells that comprises, at least, Tcons is sourced directly from an aliquot of peripheral blood or apheresis product. In some embodiments, a population of cells can be enriched for Tcons, for example, by sorting based on the expression of various markers using MACS, FACS, or a combination thereof. In some embodiments, a third population of CD45⁺ cells can be enriched by sorting for CD3⁺ cells. In some embodiments, a third population of CD45⁺ cells can be enriched by sorting for CD4⁺ and CD8⁺ cells. In some embodiments, a third population of CD45⁺ cells can be enriched by negative selection, where non-Tcon cells are removed, for example, by MACS depletion of cells expressing CD34, CD19, CD25, or a combination thereof.

The number of Tcons present in a third population of CD45⁺ cells can be quantified, for example, by quantifying CD3⁺ cells via flow cytometry. The number of CD3⁺ cells in an aliquot can be determined and a volume comprising an appropriate dose of CD3 cells administered to the recipient. Dose calculations can be adjusted based on measures of cell viability, e.g., viability determined via flow cytometry with propidium iodide or 7-AAD, or via trypan blue exclusion.

An apheresis product of the disclosure can be split into two portions, one portion used to provide the third population of CD45⁺ cells that comprises, at least, Tcons and the other portion to isolate and purify the population of CD45⁺ cells that comprises, at least, HSPCs, and the cell population enriched for Tregs. In alternate embodiments, CD34⁺ cells are isolated and purified from the apheresis product, creating a CD34-negative cell fraction from which the cell Treg are then isolated to help provide the cell population enriched for Tregs.

A cell population of the disclosure can comprise a population of iNKTs. A population of iNKTs can be sourced from peripheral blood. A population of iNKTs can be sourced from a peripheral blood apheresis product.

A population of cells can be enriched for iNKTs, for example, by sorting based on the expression of various markers using MACS, FACS, or a combination thereof. A population of iNKTs can be enriched, for example, by sorting for CD3⁺Vα24Jα18⁺ cells.

The number of iNKTs present in a population can be quantified, for example, by quantifying CD3⁺Vα24Jα18⁺ cells via flow cytometry. The number of CD3⁺Vα24Jα18⁺ cells in an aliquot can be determined and a volume comprising an appropriate dose of iNKTs administered to the recipient. In some embodiments, dose calculations are adjusted based on measures of cell viability measurements, e.g., viability determined via flow cytometry with propidium iodide or 7-AAD, or via trypan blue exclusion.

A cell population of the disclosure can comprise a population of Tmems. A population of Tmems can be sourced from peripheral blood. A population of Tmems can be sourced from a peripheral blood apheresis product.

A population of cells can be enriched for Tmems, for example, by sorting based on the expression of various markers using MACS, FACS, or a combination thereof. A population of Tmems can be enriched, for example, by sorting for CD3⁺CD45RA−CD45RO⁺ cells.

The number of Tmems present in a population can be quantified, for example, by quantifying CD3⁺CD45RA−CD45RO⁺ cells via flow cytometry. The number of CD3⁺CD45RA−CD45RO⁺ cells in an aliquot can be determined and a volume comprising an appropriate dose of Tmems administered to the recipient. Dose calculations can be adjusted based on measures of cell viability measurements, e.g., viability determined via flow cytometry with propidium iodide or 7-AAD, or via trypan blue exclusion.

A cell population of the disclosure can be administered freshly after isolation, or after cryopreservation and subsequent thawing.

Cells freshly isolated from a donor (“fresh cells”) can be administered to a recipient subject. Fresh cells can be stored in a buffer, for example, CliniMACs PBS-EDTA Buffer with 0.5% human serum albumin, or Plasma-Lyte-A, pH 7.4 supplemented with 2% human serum albumin. Fresh cells can be stored at a reduced temperature (e.g., 2-8° C.), and without being cryopreserved/frozen.

After acquiring a fresh population of cells from a donor, the fresh cells can be stored for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 44 hours, at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to administration to a subject.

After acquiring a fresh population of cells from a donor, the fresh cells can be stored for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 12 hours, at most about 14 hours, at most about 16 hours, at most about 18 hours, at most about 20, at most 22 hours, at most about 24 hours, at most about 30 hours, at most about 36 hours, at most about 40 hours, at most about 48 hours, at most about 60 hours, at most about 70 hours, at most about 72 hours, at most about 80 hours, at most about 90 hours, at most about 96 hours, at most about 120 hours, at most about 150 hours, at most about 200 hours, or at most about 300 hours prior to administration to a subject.

In some embodiments, after processing, one or more cell populations of the present disclosure, e.g., the first population of CD45⁺ cells, the second population of CD45⁺ cells, and/or the third population of CD45⁺ cells, may be formulated with one or more excipients and/or cryoprotectants. In some embodiments, the one or more excipients comprise buffers, such as transport or infusion buffers. In some embodiments, the cell populations are formulated at a neutral pH. In some embodiments, the HSPCs of the present disclosure are formulated with one or more excipients at a neutral pH. In some embodiments, the Tregs of the present disclosure are formulated with one or more excipients at a neutral pH. In some embodiments, the Tcons of the present disclosure are formulated with one or more excipients at a neutral pH. In some embodiments, the HSPCs and Tregs of the present disclosure are formulated together with one or more excipients at a neutral pH. In some embodiments, the HSPCs and Tcons of the present disclosure are formulated together with one or more excipients at a neutral pH. In some embodiments, the Tregs and Tcons of the present disclosure are formulated together with one or more excipients at a neutral pH. In some embodiments, the HSPCs, Tregs, and Tcons of the present disclosure are formulated together with one or more excipients at a neutral pH. In some embodiments, the one or more excipients comprise one or more transport buffers.

In some embodiments, a neutral pH ranges from approximately 6.8 to approximately 7.6. In some embodiments, a neutral pH is approximately 6.8, approximately 6.85, approximately 6.9, approximately 6.95, approximately 7, approximately 7.05, approximately 7.1, approximately 7.15, approximately 7.2, approximately 7.25, approximately 7.3, approximately 7.35, approximately 7.4, approximately 7.45, approximately 7.5, approximately 7.55, or approximately 7.6.

### Donors

In some embodiments, one or more cell populations of the present disclosure, e.g., a first population of CD45⁺ cells of the present disclosure, a second population of CD45⁺ cells of the present disclosure, a third population of CD45⁺ cells of the present disclosure, and/or one or more cells comprising a chimeric receptor are derived from a single human blood donor. In embodiments, the respective cell populations are provided as separate cell populations and are derived from a single human blood donor.

A cell population and/or one or more cells comprising a chimeric receptor can comprise cells that are from one or more donors that have each been HLA typed, for example, to determine a degree of HLA matching to a subject that will receive the cell population. In some embodiments, the donor is unrelated to the recipient subject. In some embodiments, the donor is related to a recipient subject, for example, the donor is a parent, child, sibling, grandparent, grandchild, aunt, uncle, or cousin. In some embodiments, the donor is a first-degree blood relative of the recipient subject. In some embodiments, the donor is a second-degree blood relative of the recipient subject. In some embodiments, the related or unrelated donor is HLA matched to a recipient subject. In some embodiments, the related or unrelated donor is HLA mismatched to a recipient subject. In some embodiments, the related or unrelated donor is haploidentical to a recipient subject. In some embodiments, the related or unrelated donor is at least 16 years old. In some embodiments, the related or unrelated donor is at least 18 years old.

Human leukocyte antigens (HLA), also broadly referred to as Major histocompatibility complex (MHC) antigens, can be protein molecules expressed on the surface of a cell that can confer an antigenic identity to that cell. HLA/MHC antigens are target molecules that can be recognized by T cells and natural killer (NK) cells as being derived from the same source of hematopoietic stem cells as the immune effector cells (“self”), or as being derived from another source of hematopoietic cells (“non-self”). HLA class I antigens (A, B, and C in humans) can be expressed by the vast majority of cells, while HLA class II antigens (DR, DP, and DQ in humans) can be expressed primarily on professional antigen presenting cells. Both HLA classes can be implicated in GVHD.

HLA antigens are encoded by highly polymorphic genes; a range of alleles exist for each HLA class I and II gene. Allelic gene products can differ in one or more amino acids in the α and/or β domain(s). Panels of specific antibodies or nucleic acid reagents can be used to determine HLA haplotypes of individuals, for example, using leukocytes that express class I and class II molecules. HLA alleles can be described at various levels of detail. Most designations begin with HLA- and the locus name, then * and some (even) number of digits specifying the allele. The first two digits can specify a group of alleles. The third through fourth digits, when present, can specify a synonymous allele. Digits five through six, when present, can denote any synonymous mutations within the coding frame of the gene. The seventh and eighth digits, when present, can distinguish mutations outside the coding region. Letters such as L, N, Q, or S may follow an allele's designation to specify an expression level or other non-genomic data known about it. Thus, a completely described allele may be up to 9 digits long, not including the HLA-prefix and locus notation.

The set of HLA alleles inherited from one parent forms a haplotype. HLA haploidentical can refer to a donor-recipient pair where one chromosome is matched at least at HLA-A; HLA-B, and HLA-DR between the donor and recipient. The haploidentical pair may or may not be matched at other alleles, e.g., other HLA genes on the other chromosome, or additional histocompatibility loci on either chromosome. Such donors can frequently occur in families, e.g., a parent can be haploidentical to a child; and siblings may be haploidentical.

A cell population can be from a related or unrelated donor that has been HLA-typed at any number of HLA alleles. A donor and a subject can be HLA matched, e.g., matched at all typed HLA alleles. A donor and a subject can be HLA mismatched, e.g., at least one HLA antigen can be mismatched between the donor and recipient.

In some embodiments, a related or unrelated donor and a subject can be HLA-typed at six alleles, for example, HLA-A, HLA-B, and HLA-DR alleles. The donor and subject can be matched at, for example 3/6 4/6, 5/6, or 6/6 of the alleles. In some embodiments, the donor and subject are matched at least at 5/6 alleles. In some embodiments, the donor and subject are matched at 6/6 alleles.

In some embodiments, a related or unrelated donor and a subject can be HLA-typed at eight alleles, for example, HLA-A, HLA-B, HLA-C, and HLA-DR alleles (e.g., HLA-DRB1 alleles). The donor and subject can be matched at, for example 4/8, 5/8, 6/8, 7/8, or 8/8 of the alleles. In some embodiments, the donor and subject are matched at least at 6/8 alleles. In some embodiments, the donor and subject are matched at least at 7/8 alleles. In some embodiments, the donor and subject are matched at 8/8 alleles.

In some embodiments, a related or unrelated donor and a subject can be HLA-typed at ten alleles, for example, HLA-A, HLA-B, HLA-C, and HLA-DR alleles (e.g., HLA-DRB1 alleles). The donor and subject can be matched at, for example 5/10, 6/10, 7/10, 8/10, 9/10, or 10/10 of the alleles. In some embodiments, the donor and subject are matched at least at 7/10 alleles. In some embodiments, the donor and subject are matched at least at 8/10 alleles. In some embodiments, the donor and subject are matched at least at 9/10 alleles. In some embodiments, the donor and subject are matched at 10/10 alleles.

In some embodiments, a related or unrelated donor and a subject can be HLA-typed at twelve alleles, for example, HLA-A, HLA-B, HLA-C, HLA-DR alleles (e.g., HLA-DRB1 alleles), and HLA-DP alleles (e.g., HLA-DPB1 alleles). The donor and subject can be matched at, for example 6/12, 7/12, 8/12, 9/12, 10/12, 11/12, or 12/12 of the alleles. In some embodiments, the donor and subject are matched at least at 9/12 alleles. In some embodiments, the donor and subject are matched at least at 10/12 alleles. In some embodiments, the donor and subject are matched at least at 11/12 alleles. In some embodiments, the donor and subject are matched at 12/12 alleles.

A cell population and/or one or more cells comprising a chimeric receptor can be generated from a matched unrelated donor that is an 8/8 match for HLA-A, -B, -C, and -DRB1, all typed using DNA-based high-resolution methods. A cell population can be generated from a matched unrelated donor that is an 7/8 match for HLA-A, -B, -C, and -DRB1, all typed using DNA-based high-resolution methods. A cell population can be generated from a matched unrelated donor that is an 6/8 match for HLA-A, -B, -C, and -DRB1, all typed using DNA-based high-resolution methods. A cell population can be generated from a matched unrelated donor that is an 10/10 match for HLA-A, -B, -C, -DQB1 and -DRB1, all typed using DNA-based high-resolution methods. A cell population can be generated from a matched unrelated donor that is an 9/10 match for HLA-A, -B, -C, -DQB1 and -DRB1, all typed using DNA-based high-resolution methods. A cell population can be generated from a matched unrelated donor that is an 8/10 match for HLA-A, -B, -C, -DQB1 and -DRB1, all typed using DNA-based high-resolution methods. A cell population can be generated from a matched unrelated donor that is an 7/10 match for HLA-A, -B, -C, -DQB1 and -DRB1, all typed using DNA-based high-resolution methods. A cell population can be generated from a matched unrelated donor that is an 12/12 match for HLA-A, -B, -C, -DQB1, -DRB1, and -DPB1, all typed using DNA-based high-resolution methods. A cell population can be generated from a matched unrelated donor that is an 11/12 match for HLA-A, -B, -C, -DQB1, -DRB1, and -DPB1, all typed using DNA-based high-resolution methods. A cell population can be generated from a matched unrelated donor that is an 10/12 match for HLA-A, -B, -C, -DQB1, -DRB1, and -DPB1, all typed using DNA-based high-resolution methods. A cell population can be generated from a matched unrelated donor that is an 9/12 match for HLA-A, -B, -C, -DQB1, -DRB1, and -DPB1, all typed using DNA-based high-resolution methods.

A cell population and/or one or more cells comprising a chimeric receptor can be generated from a matched related donor that is an 8/8 match for HLA-A, -B, -C, and -DRB1, all typed using DNA-based high-resolution methods. A cell population can be generated from a matched related donor that is an 7/8 match for HLA-A, -B, -C, and -DRB1, all typed using DNA-based high-resolution methods. A cell population can be generated from a matched related donor that is an 6/8 match for HLA-A, -B, -C, and -DRB1, all typed using DNA-based high-resolution methods. A cell population can be generated from a matched related donor that is an 10/10 match for HLA-A, -B, -C, -DQB1, and -DRB1, all typed using DNA-based high-resolution methods. A cell population can be generated from a matched related donor that is an 9/10 match for HLA-A, -B, -C, -DQB1, and -DRB1, all typed using DNA-based high-resolution methods. A cell population can be generated from a matched related donor that is an 8/10 match for HLA-A, -B, -C, -DQB1, and -DRB1, all typed using DNA-based high-resolution methods. A cell population can be generated from a matched related donor that is an 7/10 match for HLA-A, -B, -C, -DQB1, and -DRB1, all typed using DNA-based high-resolution methods. A cell population can be generated from a matched related donor that is an 12/12 match for HLA-A, -B, -C, -DQB1, -DRB1, and -DPB1, all typed using DNA-based high-resolution methods. A cell population can be generated from a matched related donor that is an 11/12 match for HLA-A, -B, -C, -DQB1, -DRB1, and -DPB1, all typed using DNA-based high-resolution methods. A cell population can be generated from a matched related donor that is an 10/12 match for HLA-A, -B, -C, -DQB1, -DRB1, and -DPB1, all typed using DNA-based high-resolution methods. A cell population can be generated from a matched related donor that is an 9/12 match for HLA-A, -B, -C, -DQB1, -DRB1, and -DPB1, all typed using DNA-based high-resolution methods.

A cell population and/or one or more cells comprising a chimeric receptor can be generated from a matched parent donor that is an 8/8 match for HLA-A, -B, -C, and -DRB1, all typed using DNA-based high-resolution methods. A cell population can be generated from a matched parent donor that is an 7/8 match for HLA-A, -B, -C, and -DRB1, all typed using DNA-based high-resolution methods. A cell population can be generated from a matched parent donor that is an 6/8 match for HLA-A, -B, -C, and -DRB1, all typed using DNA-based high-resolution methods. A cell population can be generated from a matched parent donor that is an 10/10 match for HLA-A, -B, -C, -DQB1, and -DRB1, all typed using DNA-based high-resolution methods. A cell population can be generated from a matched parent donor that is an 9/10 match for HLA-A, -B, -C, -DQB1, and -DRB1, all typed using DNA-based high-resolution methods. A cell population can be generated from a matched parent donor that is an 8/10 match for HLA-A, -B, -C, -DQB1, and -DRB1, all typed using DNA-based high-resolution methods. A cell population can be generated from a matched parent donor that is an 7/10 match for HLA-A, -B, -C, -DQB1, and -DRB1, all typed using DNA-based high-resolution methods. A cell population can be generated from a matched parent donor that is an 12/12 match for HLA-A, -B, -C, -DQB1, -DRB1, and -DPB1, all typed using DNA-based high-resolution methods. A cell population can be generated from a matched parent donor that is an 11/12 match for HLA-A, -B, -C, -DQB1, -DRB1, and -DPB1, all typed using DNA-based high-resolution methods. A cell population can be generated from a matched parent donor that is an 10/12 match for HLA-A, -B, -C, -DQB1, -DRB1, and -DPB1, all typed using DNA-based high-resolution methods. A cell population can be generated from a matched parent donor that is an 9/12 match for HLA-A, -B, -C, -DQB1, -DRB1, and -DPB1, all typed using DNA-based high-resolution methods.

A cell population and/or one or more cells comprising a chimeric receptor can be generated from a matched child donor that is an 8/8 match for HLA-A, -B, -C, and -DRB1, all typed using DNA-based high-resolution methods. A cell population can be generated from a matched child donor that is an 7/8 match for HLA-A, -B, -C, and -DRB1, all typed using DNA-based high-resolution methods. A cell population can be generated from a matched child donor that is an 6/8 match for HLA-A, -B, -C, and -DRB1, all typed using DNA-based high-resolution methods. A cell population can be generated from a matched child donor that is an 10/10 match for HLA-A, -B, -C, -DQB1, and -DRB1, all typed using DNA-based high-resolution methods. A cell population can be generated from a matched child donor that is an 9/10 match for HLA-A, -B, -C, -DQB1, and -DRB1, all typed using DNA-based high-resolution methods. A cell population can be generated from a matched child donor that is an 8/10 match for HLA-A, -B, -C, -DQB1, and -DRB1, all typed using DNA-based high-resolution methods. A cell population can be generated from a matched child donor that is an 7/10 match for HLA-A, -B, -C, -DQB1, and -DRB1, all typed using DNA-based high-resolution methods. A cell population can be generated from a matched child donor that is an 12/12 match for HLA-A, -B, -C, -DQB1, -DRB1, and -DPB1, all typed using DNA-based high-resolution methods. A cell population can be generated from a matched child donor that is an 11/12 match for HLA-A, -B, -C, -DQB1, -DRB1, and -DPB1, all typed using DNA-based high-resolution methods. A cell population can be generated from a matched child donor that is an 10/12 match for HLA-A, -B, -C, -DQB1, -DRB1, and -DPB1, all typed using DNA-based high-resolution methods. A cell population can be generated from a matched child donor that is an 9/12 match for HLA-A, -B, -C, -DQB1, -DRB1, and -DPB1, all typed using DNA-based high-resolution methods.

A cell population and/or one or more cells comprising a chimeric receptor can be generated from a matched sibling donor that is an 8/8 match for HLA-A, -B, -C, and -DRB1, all typed using DNA-based high-resolution methods. A cell population can be generated from a matched sibling donor that is an 7/8 match for HLA-A, -B, -C, and -DRB1, all typed using DNA-based high-resolution methods. A cell population can be generated from a matched sibling donor that is an 6/8 match for HLA-A, -B, -C, and -DRB1, all typed using DNA-based high-resolution methods. A cell population can be generated from a matched sibling donor that is an 10/10 match for HLA-A, -B, -C, -DQB1 and -DRB1, all typed using DNA-based high-resolution methods. A cell population can be generated from a matched sibling donor that is an 9/10 match for HLA-A, -B, -C, -DQB1 and -DRB1, all typed using DNA-based high-resolution methods. A cell population can be generated from a matched sibling donor that is an 8/10 match for HLA-A, -B, -C, -DQB1 and -DRB1, all typed using DNA-based high-resolution methods. A cell population can be generated from a matched sibling donor that is an 7/10 match for HLA-A, -B, -C, -DQB1 and -DRB1, all typed using DNA-based high-resolution methods. A cell population can be generated from a matched child donor that is an 12/12 match for HLA-A, -B, -C, -DQB1, -DRB1, and -DPB1, all typed using DNA-based high-resolution methods. A cell population can be generated from a matched child donor that is an 11/12 match for HLA-A, -B, -C, -DQB1, -DRB1, and -DPB1, all typed using DNA-based high-resolution methods. A cell population can be generated from a matched child donor that is an 10/12 match for HLA-A, -B, -C, -DQB1, -DRB1, and -DPB1, all typed using DNA-based high-resolution methods. A cell population can be generated from a matched child donor that is an 9/12 match for HLA-A, -B, -C, -DQB1, -DRB1, and -DPB1, all typed using DNA-based high-resolution methods.

A cell population and/or one or more cells comprising a chimeric receptor can be generated from a matched grandparent donor that is an 8/8 match for HLA-A, -B, -C, and -DRB1, all typed using DNA-based high-resolution methods. A cell population can be generated from a matched grandparent donor that is an 7/8 match for HLA-A, -B, -C, and -DRB1, all typed using DNA-based high-resolution methods. A cell population can be generated from a matched grandparent donor that is an 6/8 match for HLA-A, -B, -C, and -DRB1, all typed using DNA-based high-resolution methods. A cell population can be generated from a matched grandparent donor that is an 10/10 match for HLA-A, -B, -C, -DQB1, and -DRB1, all typed using DNA-based high-resolution methods. A cell population can be generated from a matched grandparent donor that is an 9/10 match for HLA-A, -B, -C, -DQB1, and -DRB1, all typed using DNA-based high-resolution methods. A cell population can be generated from a matched grandparent donor that is an 8/10 match for HLA-A, -B, -C, -DQB1, and -DRB1, all typed using DNA-based high-resolution methods. A cell population can be generated from a matched grandparent donor that is an 7/10 match for HLA-A, -B, -C, -DQB1, and -DRB1, all typed using DNA-based high-resolution methods. A cell population can be generated from a matched grandparent donor that is an 12/12 match for HLA-A, -B, -C, -DQB1, -DRB1, and -DPB1, all typed using DNA-based high-resolution methods. A cell population can be generated from a matched grandparent donor that is an 11/12 match for HLA-A, -B, -C, -DQB1, -DRB1, and -DPB1, all typed using DNA-based high-resolution methods. A cell population can be generated from a matched grandparent donor that is an 10/12 match for HLA-A, -B, -C, -DQB1, -DRB1, and -DPB1, all typed using DNA-based high-resolution methods. A cell population can be generated from a matched grandparent donor that is an 9/12 match for HLA-A, -B, -C, -DQB1, -DRB1, and -DPB1, all typed using DNA-based high-resolution methods.

A cell population and/or one or more cells comprising a chimeric receptor can be generated from a matched grandchild donor that is an 8/8 match for HLA-A, -B, -C, and -DRB1, all typed using DNA-based high-resolution methods. A cell population can be generated from a matched grandchild donor that is an 7/8 match for HLA-A, -B, -C, and -DRB1, all typed using DNA-based high-resolution methods. A cell population can be generated from a matched grandchild donor that is an 6/8 match for HLA-A, -B, -C, and -DRB1, all typed using DNA-based high-resolution methods. A cell population can be generated from a matched grandchild donor that is an 10/10 match for HLA-A, -B, -C, -DQB1, and -DRB1, all typed using DNA-based high-resolution methods. A cell population can be generated from a matched grandchild donor that is an 9/10 match for HLA-A, -B, -C, -DQB1, and -DRB1, all typed using DNA-based high-resolution methods. A cell population can be generated from a matched grandchild donor that is an 8/10 match for HLA-A, -B, -C, -DQB1, and -DRB1, all typed using DNA-based high-resolution methods. A cell population can be generated from a matched grandchild donor that is an 7/10 match for HLA-A, -B, -C, -DQB1, and -DRB1, all typed using DNA-based high-resolution methods. A cell population can be generated from a matched grandchild donor that is an 12/12 match for HLA-A, -B, -C, -DQB1, -DRB1, and -DPB1, all typed using DNA-based high-resolution methods. A cell population can be generated from a matched grandchild donor that is an 11/12 match for HLA-A, -B, -C, -DQB1, -DRB1, and -DPB1, all typed using DNA-based high-resolution methods. A cell population can be generated from a matched grandchild donor that is an 10/12 match for HLA-A, -B, -C, -DQB1, -DRB1, and -DPB1, all typed using DNA-based high-resolution methods. A cell population can be generated from a matched grandchild donor that is an 9/12 match for HLA-A, -B, -C, -DQB1, -DRB1, and -DPB1, all typed using DNA-based high-resolution methods.

A cell population and/or one or more cells comprising a chimeric receptor can be generated from a matched aunt donor that is an 8/8 match for HLA-A, -B, -C, and -DRB1, all typed using DNA-based high-resolution methods. A cell population can be generated from a matched aunt donor that is an 7/8 match for HLA-A, -B, -C, and -DRB1, all typed using DNA-based high-resolution methods. A cell population can be generated from a matched aunt donor that is an 6/8 match for HLA-A, -B, -C, and -DRB1, all typed using DNA-based high-resolution methods. A cell population can be generated from a matched aunt donor that is an 10/10 match for HLA-A, -B, -C, -DQB1, and -DRB1, all typed using DNA-based high-resolution methods. A cell population can be generated from a matched aunt donor that is an 9/10 match for HLA-A, -B, -C, -DQB1, and -DRB1, all typed using DNA-based high-resolution methods. A cell population can be generated from a matched aunt donor that is an 8/10 match for HLA-A, -B, -C, -DQB1, and -DRB1, all typed using DNA-based high-resolution methods. A cell population can be generated from a matched aunt donor that is an 7/10 match for HLA-A, -B, -C, -DQB1, and -DRB1, all typed using DNA-based high-resolution methods. A cell population can be generated from a matched aunt donor that is an 12/12 match for HLA-A, -B, -C, -DQB1, -DRB1, and -DPB1, all typed using DNA-based high-resolution methods. A cell population can be generated from a matched aunt donor that is an 11/12 match for HLA-A, -B, -C, -DQB1, -DRB1, and -DPB1, all typed using DNA-based high-resolution methods. A cell population can be generated from a matched aunt donor that is an 10/12 match for HLA-A, -B, -C, -DQB1, -DRB1, and -DPB1, all typed using DNA-based high-resolution methods. A cell population can be generated from a matched aunt donor that is an 9/12 match for HLA-A, -B, -C, -DQB1, -DRB1, and -DPB1, all typed using DNA-based high-resolution methods.

A cell population and/or one or more cells comprising a chimeric receptor can be generated from a matched uncle donor that is an 8/8 match for HLA-A, -B, -C, and -DRB1, all typed using DNA-based high-resolution methods. A cell population can be generated from a matched uncle donor that is an 7/8 match for HLA-A, -B, -C, and -DRB1, all typed using DNA-based high-resolution methods. A cell population can be generated from a matched uncle donor that is an 6/8 match for HLA-A, -B, -C, and -DRB1, all typed using DNA-based high-resolution methods. A cell population can be generated from a matched uncle donor that is an 10/10 match for HLA-A, -B, -C, -DQB1, and -DRB1, all typed using DNA-based high-resolution methods. A cell population can be generated from a matched uncle donor that is an 9/10 match for HLA-A, -B, -C, -DQB1, and -DRB1, all typed using DNA-based high-resolution methods. A cell population can be generated from a matched uncle donor that is an 8/10 match for HLA-A, -B, -C, -DQB1, and -DRB1, all typed using DNA-based high-resolution methods. A cell population can be generated from a matched uncle donor that is an 7/10 match for HLA-A, -B, -C, -DQB1, and -DRB1, all typed using DNA-based high-resolution methods. A cell population can be generated from a matched uncle donor that is an 12/12 match for HLA-A, -B, -C, -DQB1, -DRB1, and -DPB1, all typed using DNA-based high-resolution methods. A cell population can be generated from a matched uncle donor that is an 11/12 match for HLA-A, -B, -C, -DQB1, -DRB1, and -DPB1, all typed using DNA-based high-resolution methods. A cell population can be generated from a matched uncle donor that is an 10/12 match for HLA-A, -B, -C, -DQB1, -DRB1, and -DPB1, all typed using DNA-based high-resolution methods. A cell population can be generated from a matched uncle donor that is an 9/12 match for HLA-A, -B, -C, -DQB1, -DRB1, and -DPB1, all typed using DNA-based high-resolution methods.

A cell population and/or one or more cells comprising a chimeric receptor can be generated from a matched cousin donor that is an 8/8 match for HLA-A, -B, -C, and -DRB1, all typed using DNA-based high-resolution methods. A cell population can be generated from a matched cousin donor that is an 7/8 match for HLA-A, -B, -C, and -DRB1, all typed using DNA-based high-resolution methods. A cell population can be generated from a matched cousin donor that is an 6/8 match for HLA-A, -B, -C, and -DRB1, all typed using DNA-based high-resolution methods. A cell population can be generated from a matched cousin donor that is an 10/10 match for HLA-A, -B, -C, -DQB1, and -DRB1, all typed using DNA-based high-resolution methods. A cell population can be generated from a matched cousin donor that is an 9/10 match for HLA-A, -B, -C, -DQB1, and -DRB1, all typed using DNA-based high-resolution methods. A cell population can be generated from a matched cousin donor that is an 8/10 match for HLA-A, -B, -C, -DQB1, and -DRB1, all typed using DNA-based high-resolution methods. A cell population can be generated from a matched cousin donor that is an 7/10 match for HLA-A, -B, -C, -DQB1, and -DRB1, all typed using DNA-based high-resolution methods. A cell population can be generated from a matched cousin donor that is an 12/12 match for HLA-A, -B, -C, -DQB1, -DRB1, and -DPB1, all typed using DNA-based high-resolution methods. A cell population can be generated from a matched cousin donor that is an 11/12 match for HLA-A, -B, -C, -DQB1, -DRB1, and -DPB1, all typed using DNA-based high-resolution methods. A cell population can be generated from a matched cousin donor that is an 10/12 match for HLA-A, -B, -C, -DQB1, -DRB1, and -DPB1, all typed using DNA-based high-resolution methods. A cell population can be generated from a matched cousin donor that is an 9/12 match for HLA-A, -B, -C, -DQB1, -DRB1, and -DPB1, all typed using DNA-based high-resolution methods.

In some embodiments, the HLA mismatch occurs as the result of the allogeneic related or unrelated donor being homozygous for the HLA allele while the recipient subject is heterogeneous for the HLA allele. In some embodiments, the HLA mismatch occurs as the result of the allogeneic related or unrelated donor being heterogeneous for the HLA allele while the recipient subject is homozygous for the HLA allele. In some embodiments, the HLA mismatch occurs as the result of both the allogeneic related or unrelated donor and the recipient subject being heterozygous for the HLA allele.

In various embodiments, the first population of CD45⁺ cells, the population of cells enriched for Tregs (e.g., the second population of CD45⁺ cells), the third population of CD45⁺ cells, and/or the one or more cells comprising a chimeric receptor is allogeneic relative to the human subject.

In some embodiments, the first population of CD45⁺ cells, the population of cells enriched for Tregs (e.g., the second population of CD45⁺ cells), the third population of CD45⁺ cells, and/or the one or more cells comprising a chimeric receptor is obtained from a donor that is HLA-matched relative to the human subject.

In embodiments, the first population of CD45⁺ cells, the population of cells enriched for Tregs (e.g., the second population of CD45⁺ cells), the third population of CD45⁺ cells, and/or the one or more cells comprising a chimeric receptor is obtained from a donor that is HLA-mismatched relative to the human subject.

In various embodiments, the first population of CD45⁺ cells, the population of cells enriched for Tregs (e.g., the second population of CD45⁺ cells), the third population of CD45⁺ cells, and/or the one or more cells comprising a chimeric receptor is obtained from a donor that is haploidentical relative to the human subject.

A cell population and/or one or more cells comprising a chimeric receptor can be derived from an allogeneic donor. A cell population and/or one or more cells comprising a chimeric receptor can be generated from a donor that is a first-degree blood relative of the subject. A cell population and/or one or more cells comprising a chimeric receptor can be generated from a donor that is a second-degree blood relative of the subject. A cell population and/or one or more cells comprising a chimeric receptor can be generated from a donor that is not related to the subject. A cell population and/or one or more cells comprising a chimeric receptor can be generated from a donor that is HLA matched to a recipient subject. A cell population and/or one or more cells comprising a chimeric receptor can be generated from a donor that is HLA mismatched to a recipient subject. A cell population and/or one or more cells comprising a chimeric receptor can be generated from a donor that is haploidentical to a recipient subject. A cell population and/or one or more cells comprising a chimeric receptor can be generated from a donor that is related to a recipient subject, for example, a parent, child, sibling, grandparent, grandchild, aunt, uncle, or cousin. A cell population and/or one or more cells comprising a chimeric receptor can be generated from a donor that is at least 16 years old. A cell population can be generated from a donor that is at least 18 years old.

A cell population and/or one or more cells comprising a chimeric receptor can be generated from a donor that meets eligibility criteria for donors of viable, leukocyte-rich cells or tissues as defined by 21 CFR § 1271 2018 and relevant FDA Guidance for Industry. For example, a cell population can be generated from a donor that meets eligibility criteria outlined in any one or more of the following: Eligibility Determination for Donors of Human Cells, Tissues, and Cellular and Tissue-Based Products, 2007; Use of Donor Screening Tests to Test Donors of Human Cells, Tissues and Cellular and Tissue-Based Products for Infection with Treponema pallidum (Syphilis), 2015; Use of Nucleic Acid Tests to Reduce the Risk of Transmission of Hepatitis B Virus from Donors of Human Cells, Tissues, and Cellular and Tissue-Based Products, 2016; Use of Nucleic Acid Tests to Reduce the Risk of Transmission of West Nile Virus from Living Donors of Human Cells, Tissues, and Cellular and Tissue-Based Products (HCT/Ps), 2016; and Donor Screening Recommendations to Reduce the Risk of Transmission of Zika Virus by Human Cells, Tissues, and Cellular and Tissue-Based Products, 2018). A cell population can be generated from a donor that meets any criteria for donation as specified by standard NMDP guidelines (NMDP donors).

A cell population and/or one or more cells comprising a chimeric receptor can be generated from a donor that does not exhibit evidence of active infection. A cell population can be generated from a donor that is not seropositive for HIV-1 or -2, HTLV-1 or -2. A cell population and/or one or more cells comprising a chimeric receptor can be generated from a donor that is not positive for anti-hepatitis C (HCV) antibody or HCV NAT. A cell population and/or one or more cells comprising a chimeric receptor can be generated from a donor that tests negative for chronic HBV infection. A cell population and/or one or more cells comprising a chimeric receptor can be generated from a donor that does not have high potential for Zika virus infection as defined as any of the following: (i) Medical diagnosis of Zika virus infection in the past 6 months; (ii) Residence in, or travel to, an area with active Zika virus transmission within the past 6 months; (iii) Unprotected sex within the past 6 months with a person who is known to have either of the risk factors (i) or (ii). A cell population and/or one or more cells comprising a chimeric receptor can be generated from a donor that does not have signs or symptoms consistent with active Zika virus infection.

One or more cell populations and/or one or more cells comprising a chimeric receptor of the disclosure can be obtained from a single donor, for example, obtained from mobilized peripheral blood apheresis of a single donor. HSPCs, Tregs, Tcons, iNKTs, Tmems, or any combination thereof can be obtained from a single donor.

One or more cell populations and/or one or more cells comprising a chimeric receptor of the disclosure can be obtained from one donor, and one or more additional cell populations and/or one or more cells comprising a chimeric receptor of the disclosure can be obtained from a second donor. One cell population and/or one or more cells comprising a chimeric receptor of the disclosure can be obtained from a single donor, and a second cell population and/or one or more cells comprising a chimeric receptor of the disclosure can be obtained from multiple donors. Populations of the disclosure and/or one or more cells comprising a chimeric receptor can be obtained from multiple donors, for example, obtained from mobilized peripheral blood apheresis of multiple donors. HSPCs can be obtained from multiple donors. Tregs can be obtained from multiple donors. Tcons can be obtained from multiple donors. One or more cells comprising a chimeric receptor can be obtained from multiple donors. iNKTs can be obtained from multiple donors. Tmems can be obtained from multiple donors.

### Doses of Cell Populations

Doses of cell populations and/or one or more cells comprising a chimeric receptor administered to a subject (e.g., human subject) may be based on the subject's body weight. In some cases, a subject's body weight may be used to determine a dose of one or more cell populations to be administered to the subject. In some cases, a cell dose may be based on the ideal body weight of the subject instead of their actual weight, e.g., actual body weight. Ideal body weight may be a preferable method of dose calculation to avoid erroneous cell doses due to excess body fat and/or muscle mass. A subject's ideal body weight may be calculated using their height and sex. Other methods that calculate a subject's ideal body weight may be used. For instance, other methods which determine a subject's body fat percentage. A dose of one or more cell populations of the present disclosure, e.g., the first population of CD45⁺ cells, the second population of CD45⁺ cells, and/or the third population of CD45⁺ cells, may be based on the subject's adjusted body weight (ABW), if the subject's actual body weight is greater than 120% of his/her ideal body weight (IBW).

In some embodiments the human subject has a body weight that ranges from approximately 4 kilograms (kg) to approximately 200 kilograms (kg). In some embodiments, the human subject has a body weight that is approximately 4 kilograms, approximately 5 kilograms, approximately 6 kilograms, approximately 7 kilograms, approximately 8 kilograms, approximately 9 kilograms, approximately 10 kilograms, approximately 11 kilograms, approximately 12 kilograms, approximately 13 kilograms, approximately 14 kilograms, approximately 15 kilograms, approximately 16 kilograms, approximately 17 kilograms, approximately 18 kilograms, approximately 19 kilograms, approximately 20 kilograms, approximately 21 kilograms, approximately 22 kilograms, approximately 23 kilograms, approximately 24 kilograms, approximately 25 kilograms, approximately 26 kilograms, approximately 27 kilograms, approximately 28 kilograms, approximately 29 kilograms, approximately 30 kilograms, approximately 31 kilograms, approximately 32 kilograms, approximately 33 kilograms, approximately 34 kilograms, approximately 35 kilograms, approximately 36 kilograms, approximately 37 kilograms, approximately 38 kilograms, approximately 39 kilograms, approximately 40 kilograms, approximately 41 kilograms, approximately 42 kilograms, approximately 43 kilograms, approximately 44 kilograms, approximately 45 kilograms, approximately 46 kilograms, approximately 47 kilograms, approximately 48 kilograms, approximately 49 kilograms, approximately 50 kilograms, approximately 51 kilograms, approximately 52 kilograms, approximately 53 kilograms, approximately 54 kilograms, approximately 55 kilograms, approximately 56 kilograms, approximately 57 kilograms, approximately 58 kilograms, approximately 59 kilograms, approximately 60 kilograms, approximately 61 kilograms, approximately 62 kilograms, approximately 63 kilograms, approximately 64 kilograms, approximately 65 kilograms, approximately 66 kilograms, approximately 67 kilograms, approximately 68 kilograms, approximately 69 kilograms, approximately 70 kilograms, approximately 71 kilograms, approximately 72 kilograms, approximately 73 kilograms, approximately 74 kilograms, approximately 75 kilograms, approximately 76 kilograms, approximately 77 kilograms, approximately 78 kilograms, approximately 79 kilograms, approximately 80 kilograms, approximately 81 kilograms, approximately 82 kilograms, approximately 83 kilograms, approximately 84 kilograms, approximately 85 kilograms, approximately 86 kilograms, approximately 87 kilograms, approximately 88 kilograms, approximately 89 kilograms, approximately 90 kilograms, approximately 91 kilograms, approximately 92 kilograms, approximately 93 kilograms, approximately 94 kilograms, approximately 95 kilograms, approximately 96 kilograms, approximately 97 kilograms, approximately 98 kilograms, approximately 99 kilograms, approximately 100 kilograms, approximately 105 kilograms, approximately 110 kilograms, approximately 115 kilograms, approximately 120 kilograms, approximately 125 kilograms, approximately 130 kilograms, approximately 135 kilograms, approximately 140 kilograms, approximately 145 kilograms, approximately 150 kilograms, approximately 155 kilograms, approximately 160 kilograms, approximately 165 kilograms, approximately 170 kilograms, approximately 175 kilograms, approximately 180 kilograms, approximately 185 kilograms, approximately 190 kilograms, approximately 195 kilograms, or approximately 200 kilograms.

### HSPCs

A first population of CD45⁺ cells which comprises, at least, HSPCs or at least one dose of HSPCs, or a population of CD34⁺ hematopoietic stem and progenitor cells (HSPCs), can comprise at least about 1×10⁴, at least about 2×10⁴, at least about 3×10⁴, at least about 4×10⁴, at least about 5×10⁴, at least about 6×10⁴, at least about 7×10⁴, at least about 8×10⁴, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.1×10⁶, at least about 6.2×10⁶, at least about 6.3×10⁶, at least about 6.4×10⁶, at least about 6.5×10⁶, at least about 6.6×10⁶, at least about 6.7×10⁶, at least about 6.8×10⁶, at least about 6.9×10⁶, at least about 7×10⁶, at least about 7.1×10⁶, at least about 7.2×10⁶, at least about 7.3×10⁶, at least about 7.4×10⁶, at least about 7.5×10⁶, at least about 7.6×10⁶, at least about 7.7×10⁶, at least about 7.8×10⁶, at least about 7.9×10⁶, at least about 8×10⁶, at least about 8.1×10⁶, at least about 8.2×10⁶, at least about 8.3×10⁶, at least about 8.4×10⁶, at least about 8.5×10⁶, at least about 8.6×10⁶, at least about 8.7×10⁶, at least about 8.8×10⁶, at least about 8.9×10⁶, at least about 9×10⁶, at least about 9.1×10⁶, at least about 9.2×10⁶, at least about 9.3×10⁶, at least about 9.4×10⁶, at least about 9.5×10⁶, at least about 9.6×10⁶, at least about 9.7×10⁶, at least about 9.8×10⁶, at least about 9.9×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more cells of the first population of CD45⁺ cells, the population of HSPCs, and/or HSPCs or doses of HSPCs (e.g., CD34⁺ cells) per kilogram (kg) of recipient subject's (e.g., human subject's) actual body weight or ideal body weight.

A first population of CD45⁺ cells or a population of CD34⁺ hematopoietic stem and progenitor cells (HSPCs) can comprise at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.1×10⁶, at most about 6.2×10⁶, at most about 6.3×10⁶, at most about 6.4×10⁶, at most about 6.5×10⁶, at most about 6.6×10⁶, at most about 6.7×10⁶, at most about 6.8×10⁶, at most about 6.9×10⁶, at most about 7×10⁶, at most about 7.1×10⁶, at most about 7.2×10⁶, at most about 7.3×10⁶, at most about 7.4×10⁶, at most about 7.5×10⁶, at most about 7.6×10⁶, at most about 7.7×10⁶, at most about 7.8×10⁶, at most about 7.9×10⁶, at most about 8×10⁶, at most about 8.1×10⁶, at most about 8.2×10⁶, at most about 8.3×10⁶, at most about 8.4×10⁶, at most about 8.5×10⁶, at most about 8.6×10⁶, at most about 8.7×10⁶, at most about 8.8×10⁶, at most about 8.9×10⁶, at most about 9×10⁶, at most about 9.1×10⁶, at most about 9.2×10⁶, at most about 9.3×10⁶, at most about 9.4×10⁶, at most about 9.5×10⁶, at most about 9.6×10⁶, at most about 9.7×10⁶, at most about 9.8×10⁶, at most about 9.9×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ cells of the first population of CD45⁺ cells, the population of HSPCs, and/or HSPCs or doses of HSPCs (e.g., CD34⁺ cells) per kg of recipient subject's actual body weight or ideal body weight.

For example, a first population of CD45⁺ cells or a population of CD34⁺ hematopoietic stem and progenitor cells (HSPCs) can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ cells of the first population of CD45⁺ cells, the population of HSPCs, and/or HSPCs or doses of HSPCs (e.g., CD34⁺ cells) per kg of recipient subject's actual body weight or ideal body weight.

In some embodiments, the first population of CD45⁺ cells comprising HSPCs, the population of CD34⁺ hematopoietic stem and progenitor cells (HSPCs), or comprising at least one dose of HSPCs, comprises from approximately 1.0×10⁵ to approximately 5.0×10¹⁰, from approximately 5.0×10⁵ to approximately 1.5×10¹⁰, from approximately 5.0×10⁵ to approximately 5.0×10⁸, or from approximately 1.5×10⁷ to approximately 1.5×10¹⁰ HSPCs, e.g., in the first population of CD45⁺ cells. In some embodiments, the first population of CD45⁺ cells comprising HSPCs, or comprising at least one dose of HSPCs, comprises approximately 1.0×10⁵ or more HSPCs, approximately 2.0×10⁵ or more HSPCs, approximately 3.0×10⁵ or more HSPCs, approximately 4.0×10⁵ or more HSPCs, approximately 5.0×10⁵ or more HSPCs, approximately 6.0×10⁵ or more HSPCs, approximately 7.0×10⁵ or more HSPCs, approximately 8.0×10⁵ or more HSPCs, approximately 9.0×10⁵ or more HSPCs, approximately 1.0×10⁶ or more HSPCs, approximately 1.0×10⁶ or more HSPCs, approximately 1.1×10⁶ or more HSPCs, approximately 1.2×10⁶ or more HSPCs, approximately 1.3×10⁶ or more HSPCs, approximately 1.4×10⁶ or more HSPCs, approximately 1.5×10⁶ or more HSPCs, approximately 1.6×10⁶ or more HSPCs, approximately 1.7×10⁶ or more HSPCs, approximately 1.8×10⁶ or more HSPCs, approximately 1.9×10⁶ or more HSPCs, approximately 2.0×10⁶ or more HSPCs, approximately 2.1×10⁶ or more HSPCs, approximately 2.2×10⁶ or more HSPCs, approximately 2.3×10⁶ or more HSPCs, approximately 2.4×10⁶ or more HSPCs, approximately 2.5×10⁶ or more HSPCs, approximately 2.6×10⁶ or more HSPCs, approximately 2.7×10⁶ or more HSPCs, approximately 2.8×10⁶ or more HSPCs, approximately 2.9×10⁶ or more HSPCs, approximately 3.0×10⁶ or more HSPCs, approximately 3.1×10⁶ or more HSPCs, approximately 3.2×10⁶ or more HSPCs, approximately 3.3×10⁶ or more HSPCs, approximately 3.4×10⁶ or more HSPCs, approximately 3.5×10⁶ or more HSPCs, approximately 3.6×10⁶ or more HSPCs, approximately 3.7×10⁶ or more HSPCs, approximately 3.8×10⁶ or more HSPCs, approximately 3.9×10⁶ or more HSPCs, approximately 4.0×10⁶ or more HSPCs, approximately 4.1×10⁶ or more HSPCs, approximately 4.2×10⁶ or more HSPCs, approximately 4.3×10⁶ or more HSPCs, approximately 4.4×10⁶ or more HSPCs, approximately 4.5×10⁶ or more HSPCs, approximately 4.6×10⁶ or more HSPCs, approximately 4.7×10⁶ or more HSPCs, approximately 4.8×10⁶ or more HSPCs, approximately 4.9×10⁶ or more HSPCs, approximately 5.0×10⁶ or more HSPCs, approximately 5.1×10⁶ or more HSPCs, approximately 5.2×10⁶ or more HSPCs, approximately 5.3×10⁶ or more HSPCs, approximately 5.4×10⁶ or more HSPCs, approximately 5.5×10⁶ or more HSPCs, approximately 5.6×10⁶ or more HSPCs, approximately 5.7×10⁶ or more HSPCs, approximately 5.8×10⁶ or more HSPCs, approximately 5.9×10⁶ or more HSPCs, approximately 6.0×10⁶ or more HSPCs, approximately 6.1×10⁶ or more HSPCs, approximately 6.2×10⁶ or more HSPCs, approximately 6.3×10⁶ or more HSPCs, approximately 6.4×10⁶ or more HSPCs, approximately 6.5×10⁶ or more HSPCs, approximately 6.6×10⁶ or more HSPCs, approximately 6.7×10⁶ or more HSPCs, approximately 6.8×10⁶ or more HSPCs, approximately 6.9×10⁶ or more HSPCs, approximately 7.0×10⁶ or more HSPCs, approximately 7.1×10⁶ or more HSPCs, approximately 7.2×10⁶ or more HSPCs, approximately 7.3×10⁶ or more HSPCs, approximately 7.4×10⁶ or more HSPCs, approximately 7.5×10⁶ or more HSPCs, approximately 7.6×10⁶ or more HSPCs, approximately 7.7×10⁶ or more HSPCs, approximately 7.8×10⁶ or more HSPCs, approximately 7.9×10⁶ or more HSPCs, approximately 8.0×10⁶ or more HSPCs, approximately 8.1×10⁶ or more HSPCs, approximately 8.2×10⁶ or more HSPCs, approximately 8.3×10⁶ or more HSPCs, approximately 8.4×10⁶ or more HSPCs, approximately 8.5×10⁶ or more HSPCs, approximately 8.6×10⁶ or more HSPCs, approximately 8.7×10⁶ or more HSPCs, approximately 8.8×10⁶ or more HSPCs, approximately 8.9×10⁶ or more HSPCs, approximately 9.0×10⁶ or more HSPCs, approximately 9.1×10⁶ or more HSPCs, approximately 9.2×10⁶ or more HSPCs, approximately 9.3×10⁶ or more HSPCs, approximately 9.4×10⁶ or more HSPCs, approximately 9.5×10⁶ or more HSPCs, approximately 9.6×10⁶ or more HSPCs, approximately 9.7×10⁶ or more HSPCs, approximately 9.8×10⁶ or more HSPCs, approximately 9.9×10⁶ or more HSPCs, approximately 1.0×10⁷ or more HSPCs, approximately 1.1×10⁷ or more HSPCs, approximately 1.2×10⁷ or more HSPCs, approximately 1.3×10⁷ or more HSPCs, approximately 1.4×10⁷ or more HSPCs, approximately 1.5×10⁷ or more HSPCs, approximately 1.6×10⁷ or more HSPCs, approximately 1.7×10⁷ or more HSPCs, approximately 1.8×10⁷ or more HSPCs, approximately 1.9×10⁷ or more HSPCs, approximately 2.0×10⁷ or more HSPCs, approximately 2.1×10⁷ or more HSPCs, approximately 2.2×10⁷ or more HSPCs, approximately 2.3×10⁷ or more HSPCs, approximately 2.4×10⁷ or more HSPCs, approximately 2.5×10⁷ or more HSPCs, approximately 2.6×10⁷ or more HSPCs, approximately 2.7×10⁷ or more HSPCs, approximately 2.8×10⁷ or more HSPCs, approximately 2.9×10⁷ or more HSPCs, approximately 3.0×10⁷ or more HSPCs, approximately 3.1×10⁷ or more HSPCs, approximately 3.2×10⁷ or more HSPCs, approximately 3.3×10⁷ or more HSPCs, approximately 3.4×10⁷ or more HSPCs, approximately 3.5×10⁷ or more HSPCs, approximately 3.6×10⁷ or more HSPCs, approximately 3.7×10⁷ or more HSPCs, approximately 3.8×10⁷ or more HSPCs, approximately 3.9×10⁷ or more HSPCs, approximately 4.0×10⁷ or more HSPCs, approximately 4.1×10⁷ or more HSPCs, approximately 4.2×10⁷ or more HSPCs, approximately 4.3×10⁷ or more HSPCs, approximately 4.4×10⁷ or more HSPCs, approximately 4.5×10⁷ or more HSPCs, approximately 4.6×10⁷ or more HSPCs, approximately 4.7×10⁷ or more HSPCs, approximately 4.8×10⁷ or more HSPCs, approximately 4.9×10⁷ or more HSPCs, approximately 5.0×10⁷ or more HSPCs, approximately 5.1×10⁷ or more HSPCs, approximately 5.2×10⁷ or more HSPCs, approximately 5.3×10⁷ or more HSPCs, approximately 5.4×10⁷ or more HSPCs, approximately 5.5×10⁷ or more HSPCs, approximately 5.6×10⁷ or more HSPCs, approximately 5.7×10⁷ or more HSPCs, approximately 5.8×10⁷ or more HSPCs, approximately 5.9×10⁷ or more HSPCs, approximately 6.0×10⁷ or more HSPCs, approximately 6.1×10⁷ or more HSPCs, approximately 6.2×10⁷ or more HSPCs, approximately 6.3×10⁷ or more HSPCs, approximately 6.4×10⁷ or more HSPCs, approximately 6.5×10⁷ or more HSPCs, approximately 6.6×10⁷ or more HSPCs, approximately 6.7×10⁷ or more HSPCs, approximately 6.8×10⁷ or more HSPCs, approximately 6.9×10⁷ or more HSPCs, approximately 7.0×10⁷ or more HSPCs, approximately 7.1×10⁷ or more HSPCs, approximately 7.2×10⁷ or more HSPCs, approximately 7.3×10⁷ or more HSPCs, approximately 7.4×10⁷ or more HSPCs, approximately 7.5×10⁷ or more HSPCs, approximately 7.6×10⁷ or more HSPCs, approximately 7.7×10⁷ or more HSPCs, approximately 7.8×10⁷ or more HSPCs, approximately 7.9×10⁷ or more HSPCs, approximately 8.0×10⁷ or more HSPCs, approximately 8.1×10⁷ or more HSPCs, approximately 8.2×10⁷ or more HSPCs, approximately 8.3×10⁷ or more HSPCs, approximately 8.4×10⁷ or more HSPCs, approximately 8.5×10⁷ or more HSPCs, approximately 8.6×10⁷ or more HSPCs, approximately 8.7×10⁷ or more HSPCs, approximately 8.8×10⁷ or more HSPCs, approximately 8.9×10⁷ or more HSPCs, approximately 9.0×10⁷ or more HSPCs, approximately 9.1×10⁷ or more HSPCs, approximately 9.2×10⁷ or more HSPCs, approximately 9.3×10⁷ or more HSPCs, approximately 9.4×10⁷ or more HSPCs, approximately 9.5×10⁷ or more HSPCs, approximately 9.6×10⁷ or more HSPCs, approximately 9.7×10⁷ or more HSPCs, approximately 9.8×10⁷ or more HSPCs, approximately 9.9×10⁷ or more HSPCs, approximately 1.0×10⁸ or more HSPCs, approximately 1.1×10⁸ or more HSPCs, approximately 1.2×10⁸ or more HSPCs, approximately 1.3×10⁸ or more HSPCs, approximately 1.4×10⁸ or more HSPCs, approximately 1.5×10⁸ or more HSPCs, approximately 1.6×10⁸ or more HSPCs, approximately 1.7×10⁸ or more HSPCs, approximately 1.8×10⁸ or more HSPCs, approximately 1.9×10⁸ or more HSPCs, approximately 2.0×10⁸ or more HSPCs, approximately 2.1×10⁸ or more HSPCs, approximately 2.2×10⁸ or more HSPCs, approximately 2.3×10⁸ or more HSPCs, approximately 2.4×10⁸ or more HSPCs, approximately 2.5×10⁸ or more HSPCs, approximately 2.6×10⁸ or more HSPCs, approximately 2.7×10⁸ or more HSPCs, approximately 2.8×10⁸ or more HSPCs, approximately 2.9×10⁸ or more HSPCs, approximately 3.0×10⁸ or more HSPCs, approximately 3.1×10⁸ or more HSPCs, approximately 3.2×10⁸ or more HSPCs, approximately 3.3×10⁸ or more HSPCs, approximately 3.4×10⁸ or more HSPCs, approximately 3.5×10⁸ or more HSPCs, approximately 3.6×10⁸ or more HSPCs, approximately 3.7×10⁸ or more HSPCs, approximately 3.8×10⁸ or more HSPCs, approximately 3.9×10⁸ or more HSPCs, approximately 4.0×10⁸ or more HSPCs, approximately 4.1×10⁸ or more HSPCs, approximately 4.2×10⁸ or more HSPCs, approximately 4.3×10⁸ or more HSPCs, approximately 4.4×10⁸ or more HSPCs, approximately 4.5×10⁸ or more HSPCs, approximately 4.6×10⁸ or more HSPCs, approximately 4.7×10⁸ or more HSPCs, approximately 4.8×10⁸ or more HSPCs, approximately 4.9×10⁸ or more HSPCs, approximately 5.0×10⁸ or more HSPCs, approximately 5.1×10⁸ or more HSPCs, approximately 5.2×10⁸ or more HSPCs, approximately 5.3×10⁸ or more HSPCs, approximately 5.4×10⁸ or more HSPCs, approximately 5.5×10⁸ or more HSPCs, approximately 5.6×10⁸ or more HSPCs, approximately 5.7×10⁸ or more HSPCs, approximately 5.8×10⁸ or more HSPCs, approximately 5.9×10⁸ or more HSPCs, approximately 6.0×10⁸ or more HSPCs, approximately 6.1×10⁸ or more HSPCs, approximately 6.2×10⁸ or more HSPCs, approximately 6.3×10⁸ or more HSPCs, approximately 6.4×10⁸ or more HSPCs, approximately 6.5×10⁸ or more HSPCs, approximately 6.6×10⁸ or more HSPCs, approximately 6.7×10⁸ or more HSPCs, approximately 6.8×10⁸ or more HSPCs, approximately 6.9×10⁸ or more HSPCs, approximately 7.0×10⁸ or more HSPCs, approximately 7.1×10⁸ or more HSPCs, approximately 7.2×10⁸ or more HSPCs, approximately 7.3×10⁸ or more HSPCs, approximately 7.4×10⁸ or more HSPCs, approximately 7.5×10⁸ or more HSPCs, approximately 7.6×10⁸ or more HSPCs, approximately 7.7×10⁸ or more HSPCs, approximately 7.8×10⁸ or more HSPCs, approximately 7.9×10⁸ or more HSPCs, approximately 8.0×10⁸ or more HSPCs, approximately 8.1×10⁸ or more HSPCs, approximately 8.2×10⁸ or more HSPCs, approximately 8.3×10⁸ or more HSPCs, approximately 8.4×10⁸ or more HSPCs, approximately 8.5×10⁸ or more HSPCs, approximately 8.6×10⁸ or more HSPCs, approximately 8.7×10⁸ or more HSPCs, approximately 8.8×10⁸ or more HSPCs, approximately 8.9×10⁸ or more HSPCs, approximately 9.0×10⁸ or more HSPCs, approximately 9.1×10⁸ or more HSPCs, approximately 9.2×10⁸ or more HSPCs, approximately 9.3×10⁸ or more HSPCs, approximately 9.4×10⁸ or more HSPCs, approximately 9.5×10⁸ or more HSPCs, approximately 9.6×10⁸ or more HSPCs, approximately 9.7×10⁸ or more HSPCs, approximately 9.8×10⁸ or more HSPCs, approximately 9.9×10⁸ or more HSPCs, approximately 1.0×10⁹ or more HSPCs, approximately 1.1×10⁹ or more HSPCs, approximately 1.2×10⁹ or more HSPCs, approximately 1.3×10⁹ or more HSPCs, approximately 1.4×10⁹ or more HSPCs, approximately 1.5×10⁹ or more HSPCs, approximately 1.6×10⁹ or more HSPCs, approximately 1.7×10⁹ or more HSPCs, approximately 1.8×10⁹ or more HSPCs, approximately 1.9×10⁹ or more HSPCs, approximately 2.0×10⁹ or more HSPCs, approximately 2.1×10⁹ or more HSPCs, approximately 2.2×10⁹ or more HSPCs, approximately 2.3×10⁹ or more HSPCs, approximately 2.4×10⁹ or more HSPCs, approximately 2.5×10⁹ or more HSPCs, approximately 2.6×10⁹ or more HSPCs, approximately 2.7×10⁹ or more HSPCs, approximately 2.8×10⁹ or more HSPCs, approximately 2.9×10⁹ or more HSPCs, approximately 3.0×10⁹ or more HSPCs, approximately 3.1×10⁹ or more HSPCs, approximately 3.2×10⁹ or more HSPCs, approximately 3.3×10⁹ or more HSPCs, approximately 3.4×10⁹ or more HSPCs, approximately 3.5×10⁹ or more HSPCs, approximately 3.6×10⁹ or more HSPCs, approximately 3.7×10⁹ or more HSPCs, approximately 3.8×10⁹ or more HSPCs, approximately 3.9×10⁹ or more HSPCs, approximately 4.0×10⁹ or more HSPCs, approximately 4.1×10⁹ or more HSPCs, approximately 4.2×10⁹ or more HSPCs, approximately 4.3×10⁹ or more HSPCs, approximately 4.4×10⁹ or more HSPCs, approximately 4.5×10⁹ or more HSPCs, approximately 4.6×10⁹ or more HSPCs, approximately 4.7×10⁹ or more HSPCs, approximately 4.8×10⁹ or more HSPCs, approximately 4.9×10⁹ or more HSPCs, approximately 5.0×10⁹ or more HSPCs, approximately 5.1×10⁹ or more HSPCs, approximately 5.2×10⁹ or more HSPCs, approximately 5.3×10⁹ or more HSPCs, approximately 5.4×10⁹ or more HSPCs, approximately 5.5×10⁹ or more HSPCs, approximately 5.6×10⁹ or more HSPCs, approximately 5.7×10⁹ or more HSPCs, approximately 5.8×10⁹ or more HSPCs, approximately 5.9×10⁹ or more HSPCs, approximately 6.0×10⁹ or more HSPCs, approximately 6.1×10⁹ or more HSPCs, approximately 6.2×10⁹ or more HSPCs, approximately 6.3×10⁹ or more HSPCs, approximately 6.4×10⁹ or more HSPCs, approximately 6.5×10⁹ or more HSPCs, approximately 6.6×10⁹ or more HSPCs, approximately 6.7×10⁹ or more HSPCs, approximately 6.8×10⁹ or more HSPCs, approximately 6.9×10⁹ or more HSPCs, approximately 7.0×10⁹ or more HSPCs, approximately 7.1×10⁹ or more HSPCs, approximately 7.2×10⁹ or more HSPCs, approximately 7.3×10⁹ or more HSPCs, approximately 7.4×10⁹ or more HSPCs, approximately 7.5×10⁹ or more HSPCs, approximately 7.6×10⁹ or more HSPCs, approximately 7.7×10⁹ or more HSPCs, approximately 7.8×10⁹ or more HSPCs, approximately 7.9×10⁹ or more HSPCs, approximately 8.0×10⁹ or more HSPCs, approximately 8.1×10⁹ or more HSPCs, approximately 8.2×10⁹ or more HSPCs, approximately 8.3×10⁹ or more HSPCs, approximately 8.4×10⁹ or more HSPCs, approximately 8.5×10⁹ or more HSPCs, approximately 8.6×10⁹ or more HSPCs, approximately 8.7×10⁹ or more HSPCs, approximately 8.8×10⁹ or more HSPCs, approximately 8.9×10⁹ or more HSPCs, approximately 9.0×10⁹ or more HSPCs, approximately 9.1×10⁹ or more HSPCs, approximately 9.2×10⁹ or more HSPCs, approximately 9.3×10⁹ or more HSPCs, approximately 9.4×10⁹ or more HSPCs, approximately 9.5×10⁹ or more HSPCs, approximately 9.6×10⁹ or more HSPCs, approximately 9.7×10⁹ or more HSPCs, approximately 9.8×10⁹ or more HSPCs, approximately 9.9×10⁹ or more HSPCs, approximately 1.0×10¹⁰ or more HSPCs, approximately 1.1×10¹⁰ or more HSPCs, approximately 1.2×10¹⁰ or more HSPCs, approximately 1.3×10¹⁰ or more HSPCs, approximately 1.4×10¹⁰ or more HSPCs, approximately 1.5×10¹⁰ or more HSPCs, approximately 1.6×10¹⁰ or more HSPCs, approximately 1.7×10¹⁰ or more HSPCs, approximately 1.8×10¹⁰ or more HSPCs, approximately 1.9×10¹⁰ or more HSPCs, approximately 2.0×10¹⁰ or more HSPCs, approximately 2.1×10¹⁰ or more HSPCs, approximately 2.2×10¹⁰ or more HSPCs, approximately 2.3×10¹⁰ or more HSPCs, approximately 2.4×10¹⁰ or more HSPCs, approximately 2.5×10¹⁰ or more HSPCs, approximately 2.6×10¹⁰ or more HSPCs, approximately 2.7×10¹⁰ or more HSPCs, approximately 2.8×10¹⁰ or more HSPCs, approximately 2.9×10¹⁰ or more HSPCs, approximately 3.0×10¹⁰ or more HSPCs, approximately 3.1×10¹⁰ or more HSPCs, approximately 3.2×10¹⁰ or more HSPCs, approximately 3.3×10¹⁰ or more HSPCs, approximately 3.4×10¹⁰ or more HSPCs, approximately 3.5×10¹⁰ or more HSPCs, approximately 3.6×10¹⁰ or more HSPCs, approximately 3.7×10¹⁰ or more HSPCs, approximately 3.8×10¹⁰ or more HSPCs, approximately 3.9×10¹⁰ or more HSPCs, approximately 4.0×10¹⁰ or more HSPCs, approximately 4.1×10¹⁰ or more HSPCs, approximately 4.2×10¹⁰ or more HSPCs, approximately 4.3×10¹⁰ or more HSPCs, approximately 4.4×10¹⁰ or more HSPCs, approximately 4.5×10¹⁰ or more HSPCs, approximately 4.6×10¹⁰ or more HSPCs, approximately 4.7×10¹⁰ or more HSPCs, approximately 4.8×110¹⁰ or more HSPCs, approximately 4.9×10¹⁰ or more HSPCs, or approximately 5.0×10¹⁰ or more HSPCs.

### Tregs

A population of cells enriched for Tregs or comprising at least one dose of Tregs, e.g., a second population of CD45⁺ cells comprising at least one dose of Tregs, or a population of CD4⁺CD25⁺CD127^{dim} regulatory T cells (Tregs) can comprise at least about 1×10⁴, at least about 1×10⁵, at least about 2×10⁴, at least about 3×10⁴, at least about 4×10⁴, at least about 5×10⁴, at least about 6×10⁴, at least about 7×10⁴, at least about 8×10⁴, least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.1×10⁶, at least about 6.2×10⁶, at least about 6.3×10⁶, at least about 6.4×10⁶, at least about 6.5×10⁶, at least about 6.6×10⁶, at least about 6.7×10⁶, at least about 6.8×10⁶, at least about 6.9×10⁶, at least about 7×10⁶, at least about 7.1×10⁶, at least about 7.2×10⁶, at least about 7.3×10⁶, at least about 7.4×10⁶, at least about 7.5×10⁶, at least about 7.6×10⁶, at least about 7.7×10⁶, at least about 7.8×10⁶, at least about 7.9×10⁶, at least about 8×10⁶, at least about 8.1×10⁶, at least about 8.2×10⁶, at least about 8.3×10⁶, at least about 8.4×10⁶, at least about 8.5×10⁶, at least about 8.6×10⁶, at least about 8.7×10⁶, at least about 8.8×10⁶, at least about 8.9×10⁶, at least about 9×10⁶, at least about 9.1×10⁶, at least about 9.2×10⁶, at least about 9.3×10⁶, at least about 9.4×10⁶, at least about 9.5×10⁶, at least about 9.6×10⁶, at least about 9.7×10⁶, at least about 9.8×10⁶, at least about 9.9×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more cells of the cell population enriched for Tregs (e.g., the second population of CD45⁺ cells) and/or Tregs or doses of Tregs per kg of recipient subject's actual body weight or ideal body weight (e.g., where Tregs are CD4⁺CD25⁺CD127^{dim}, CD3⁺CD4⁺CD25⁺, CD3⁺CD4⁺CD25⁺CD127^{dim}, CD3⁺CD4⁺CD25⁺CD127^{dim} FOXP3⁺, CD3⁺FOXP3⁺, CD3⁺CD4⁺FOXP3⁺, CD3⁺CD4⁺CD25⁺FOXP3⁺, CD3⁺CD25⁺FOXP3⁺, CD3⁺CD25⁺CD127^{dim}, CD4⁺CD25⁺, CD4⁺CD25⁺CD127^{dim} FOXP3⁺, FOXP3⁺, CD4⁺FOXP3⁺, CD4⁺CD25⁺FOXP3⁺, CD25⁺FOXP3⁺, or CD25⁺CD127^{dim}).

A population of cells enriched for Tregs or comprising at least one dose of Tregs, e.g., a second population of CD45⁺ cells comprising at least one dose of Tregs, or a population of CD4⁺CD25⁺CD127^{dim} regulatory T cells (Tregs) can comprise at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.1×10⁶, at most about 6.2×10⁶, at most about 6.3×10⁶, at most about 6.4×10⁶, at most about 6.5×10⁶, at most about 6.6×10⁶, at most about 6.7×10⁶, at most about 6.8×10⁶, at most about 6.9×10⁶, at most about 7×10⁶, at most about 7.1×10⁶, at most about 7.2×10⁶, at most about 7.3×10⁶, at most about 7.4×10⁶, at most about 7.5×10⁶, at most about 7.6×10⁶, at most about 7.7×10⁶, at most about 7.8×10⁶, at most about 7.9×10⁶, at most about 8×10⁶, at most about 8.1×10⁶, at most about 8.2×10⁶, at most about 8.3×10⁶, at most about 8.4×10⁶, at most about 8.5×10⁶, at most about 8.6×10⁶, at most about 8.7×10⁶, at most about 8.8×10⁶, at most about 8.9×10⁶, at most about 9×10⁶, at most about 9.1×10⁶, at most about 9.2×10⁶, at most about 9.3×10⁶, at most about 9.4×10⁶, at most about 9.5×10⁶, at most about 9.6×10⁶, at most about 9.7×10⁶, at most about 9.8×10⁶, at most about 9.9×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ cells of the cell population enriched for Tregs (e.g., the second population of CD45⁺ cells or the population of Tregs) and/or Tregs or doses of Tregs per kg of recipient subject's actual body weight or ideal body weight (e.g., where Tregs are CD4⁺CD25⁺CD127^{dim}, CD3⁺CD4⁺CD25⁺, CD3⁺CD4⁺CD25⁺CD127^{dim}, CD3⁺CD4⁺CD25⁺CD127^{dim} FOXP3⁺, CD3⁺FOXP3⁺, CD3⁺CD4⁺FOXP3⁺, CD3⁺CD4⁺CD25⁺FOXP3⁺, CD3⁺CD25⁺FOXP3⁺, CD3⁺CD25⁺CD127^{dim}, CD4⁺CD25⁺, CD4⁺CD25⁺CD127^{dim} FOXP3⁺, FOXP3⁺, CD4⁺FOXP3⁺, CD4⁺CD25⁺FOXP3⁺, CD25⁺FOXP3⁺, or CD25⁺ CD127^{dim}).

For example, a population of cells enriched for Tregs or comprising at least one dose of Tregs, e.g., a second population of CD45⁺ cells comprising at least one dose of Tregs, or a population of CD4⁺CD25⁺CD127^{dim} regulatory T cells (Tregs) can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ cells of the cell population enriched for Tregs (e.g., the second population of CD45⁺ cells or the population of Tregs) and/or Tregs or doses of Tregs per kg of recipient subject's actual body weight or ideal body weight (e.g., where Tregs are CD4⁺CD25⁺CD127^{dim}, CD3⁺CD4⁺CD25⁺, CD3⁺CD4⁺CD25⁺CD127^{dim}, CD3⁺CD4⁺CD25⁺CD127^{dim} FOXP3⁺, CD3⁺FOXP3⁺, CD3⁺CD4⁺FOXP3⁺, CD3⁺CD4⁺CD25⁺FOXP3⁺, CD3⁺CD25⁺FOXP3⁺, CD3⁺CD25⁺CD127^{dim}, CD4⁺CD25⁺, CD4⁺CD25⁺CD127^{dim} FOXP3⁺, FOXP3⁺, CD4⁺FOXP3⁺, CD4⁺CD25⁺FOXP3⁺, CD25⁺FOXP3⁺, or CD25⁺ CD127^{dim}).

In some embodiments, the population of cells enriched for Tregs or comprising at least one dose of Tregs, e.g., a second population of CD45⁺ cells comprising at least one dose of Tregs, or a population of CD4⁺CD25⁺CD127^{dim} regulatory T cells (Tregs) comprises from approximately 1.0×10⁵ to approximately 5.0×10⁹, from approximately 5.0×10⁵ to approximately 3.0×10⁹, from approximately 1.5×10⁷ to approximately 3.0×10⁹, or from approximately 5.0×10⁵ to approximately 1.0×10⁸ Tregs, e.g., fresh isolated Tregs in the population of cells enriched for Tregs. In some embodiments, the population of cells enriched for Tregs or comprising at least one dose of Tregs, e.g., a second population of CD45+ cells comprising at least one dose of Tregs, comprises approximately 1.0×10⁵ or more Tregs, approximately 2.0×10⁵ or more Tregs, approximately 3.0×10⁵ or more Tregs, approximately 4.0×10⁵ or more Tregs, approximately 5.0×10⁵ or more Tregs, approximately 6.0×10⁵ or more Tregs, approximately 7.0×10⁵ or more Tregs, approximately 8.0×10⁵ or more Tregs, approximately 9.0×10⁵ or more Tregs, approximately 1.0×10⁶ or more Tregs, approximately 1.0×10⁶ or more Tregs, approximately 1.1×10⁶ or more Tregs, approximately 1.2×10⁶ or more Tregs, approximately 1.3×10⁶ or more Tregs, approximately 1.4×10⁶ or more Tregs, approximately 1.5×10⁶ or more Tregs, approximately 1.6×10⁶ or more Tregs, approximately 1.7×10⁶ or more Tregs, approximately 1.8×10⁶ or more Tregs, approximately 1.9×10⁶ or more Tregs, approximately 2.0×10⁶ or more Tregs, approximately 2.1×10⁶ or more Tregs, approximately 2.2×10⁶ or more Tregs, approximately 2.3×10⁶ or more Tregs, approximately 2.4×10⁶ or more Tregs, approximately 2.5×10⁶ or more Tregs, approximately 2.6×10⁶ or more Tregs, approximately 2.7×10⁶ or more Tregs, approximately 2.8×10⁶ or more Tregs, approximately 2.9×10⁶ or more Tregs, approximately 3.0×10⁶ or more Tregs, approximately 3.1×10⁶ or more Tregs, approximately 3.2×10⁶ or more Tregs, approximately 3.3×10⁶ or more Tregs, approximately 3.4×10⁶ or more Tregs, approximately 3.5×10⁶ or more Tregs, approximately 3.6×10⁶ or more Tregs, approximately 3.7×10⁶ or more Tregs, approximately 3.8×10⁶ or more Tregs, approximately 3.9×10⁶ or more Tregs, approximately 4.0×10⁶ or more Tregs, approximately 4.1×10⁶ or more Tregs, approximately 4.2×10⁶ or more Tregs, approximately 4.3×10⁶ or more Tregs, approximately 4.4×10⁶ or more Tregs, approximately 4.5×10⁶ or more Tregs, approximately 4.6×10⁶ or more Tregs, approximately 4.7×10⁶ or more Tregs, approximately 4.8×10⁶ or more Tregs, approximately 4.9×10⁶ or more Tregs, approximately 5.0×10⁶ or more Tregs, approximately 5.1×10⁶ or more Tregs, approximately 5.2×10⁶ or more Tregs, approximately 5.3×10⁶ or more Tregs, approximately 5.4×10⁶ or more Tregs, approximately 5.5×10⁶ or more Tregs, approximately 5.6×10⁶ or more Tregs, approximately 5.7×10⁶ or more Tregs, approximately 5.8×10⁶ or more Tregs, approximately 5.9×10⁶ or more Tregs, approximately 6.0×10⁶ or more Tregs, approximately 6.1×10⁶ or more Tregs, approximately 6.2×10⁶ or more Tregs, approximately 6.3×10⁶ or more Tregs, approximately 6.4×10⁶ or more Tregs, approximately 6.5×10⁶ or more Tregs, approximately 6.6×10⁶ or more Tregs, approximately 6.7×10⁶ or more Tregs, approximately 6.8×10⁶ or more Tregs, approximately 6.9×10⁶ or more Tregs, approximately 7.0×10⁶ or more Tregs, approximately 7.1×10⁶ or more Tregs, approximately 7.2×10⁶ or more Tregs, approximately 7.3×10⁶ or more Tregs, approximately 7.4×10⁶ or more Tregs, approximately 7.5×10⁶ or more Tregs, approximately 7.6×10⁶ or more Tregs, approximately 7.7×10⁶ or more Tregs, approximately 7.8×10⁶ or more Tregs, approximately 7.9×10⁶ or more Tregs, approximately 8.0×10⁶ or more Tregs, approximately 8.1×10⁶ or more Tregs, approximately 8.2×10⁶ or more Tregs, approximately 8.3×10⁶ or more Tregs, approximately 8.4×10⁶ or more Tregs, approximately 8.5×10⁶ or more Tregs, approximately 8.6×10⁶ or more Tregs, approximately 8.7×10⁶ or more Tregs, approximately 8.8×10⁶ or more Tregs, approximately 8.9×10⁶ or more Tregs, approximately 9.0×10⁶ or more Tregs, approximately 9.1×10⁶ or more Tregs, approximately 9.2×10⁶ or more Tregs, approximately 9.3×10⁶ or more Tregs, approximately 9.4×10⁶ or more Tregs, approximately 9.5×10⁶ or more Tregs, approximately 9.6×10⁶ or more Tregs, approximately 9.7×10⁶ or more Tregs, approximately 9.8×10⁶ or more Tregs, approximately 9.9×10⁶ or more Tregs, approximately 1.0×10⁷ or more Tregs, approximately 1.1×10⁷ or more Tregs, approximately 1.2×10⁷ or more Tregs, approximately 1.3×10⁷ or more Tregs, approximately 1.4×10⁷ or more Tregs, approximately 1.5×10⁷ or more Tregs, approximately 1.6×10⁷ or more Tregs, approximately 1.7×10⁷ or more Tregs, approximately 1.8×10⁷ or more Tregs, approximately 1.9×10⁷ or more Tregs, approximately 2.0×10⁷ or more Tregs, approximately 2.1×10⁷ or more Tregs, approximately 2.2×10⁷ or more Tregs, approximately 2.3×10⁷ or more Tregs, approximately 2.4×10⁷ or more Tregs, approximately 2.5×10⁷ or more Tregs, approximately 2.6×10⁷ or more Tregs, approximately 2.7×10⁷ or more Tregs, approximately 2.8×10⁷ or more Tregs, approximately 2.9×10⁷ or more Tregs, approximately 3.0×10⁷ or more Tregs, approximately 3.1×10⁷ or more Tregs, approximately 3.2×10⁷ or more Tregs, approximately 3.3×10⁷ or more Tregs, approximately 3.4×10⁷ or more Tregs, approximately 3.5×10⁷ or more Tregs, approximately 3.6×10⁷ or more Tregs, approximately 3.7×10⁷ or more Tregs, approximately 3.8×10⁷ or more Tregs, approximately 3.9×10⁷ or more Tregs, approximately 4.0×10⁷ or more Tregs, approximately 4.1×10⁷ or more Tregs, approximately 4.2×10⁷ or more Tregs, approximately 4.3×10⁷ or more Tregs, approximately 4.4×10⁷ or more Tregs, approximately 4.5×10⁷ or more Tregs, approximately 4.6×10⁷ or more Tregs, approximately 4.7×10⁷ or more Tregs, approximately 4.8×10⁷ or more Tregs, approximately 4.9×10⁷ or more Tregs, approximately 5.0×10⁷ or more Tregs, approximately 5.1×10⁷ or more Tregs, approximately 5.2×10⁷ or more Tregs, approximately 5.3×10⁷ or more Tregs, approximately 5.4×10⁷ or more Tregs, approximately 5.5×10⁷ or more Tregs, approximately 5.6×10⁷ or more Tregs, approximately 5.7×10⁷ or more Tregs, approximately 5.8×10⁷ or more Tregs, approximately 5.9×10⁷ or more Tregs, approximately 6.0×10⁷ or more Tregs, approximately 6.1×10⁷ or more Tregs, approximately 6.2×10⁷ or more Tregs, approximately 6.3×10⁷ or more Tregs, approximately 6.4×10⁷ or more Tregs, approximately 6.5×10⁷ or more Tregs, approximately 6.6×10⁷ or more Tregs, approximately 6.7×10⁷ or more Tregs, approximately 6.8×10⁷ or more Tregs, approximately 6.9×10⁷ or more Tregs, approximately 7.0×10⁷ or more Tregs, approximately 7.1×10⁷ or more Tregs, approximately 7.2×10⁷ or more Tregs, approximately 7.3×10⁷ or more Tregs, approximately 7.4×10⁷ or more Tregs, approximately 7.5×10⁷ or more Tregs, approximately 7.6×10⁷ or more Tregs, approximately 7.7×10⁷ or more Tregs, approximately 7.8×10⁷ or more Tregs, approximately 7.9×10⁷ or more Tregs, approximately 8.0×10⁷ or more Tregs, approximately 8.1×10⁷ or more Tregs, approximately 8.2×10⁷ or more Tregs, approximately 8.3×10⁷ or more Tregs, approximately 8.4×10⁷ or more Tregs, approximately 8.5×10⁷ or more Tregs, approximately 8.6×10⁷ or more Tregs, approximately 8.7×10⁷ or more Tregs, approximately 8.8×10⁷ or more Tregs, approximately 8.9×10⁷ or more Tregs, approximately 9.0×10⁷ or more Tregs, approximately 9.1×10⁷ or more Tregs, approximately 9.2×10⁷ or more Tregs, approximately 9.3×10⁷ or more Tregs, approximately 9.4×10⁷ or more Tregs, approximately 9.5×10⁷ or more Tregs, approximately 9.6×10⁷ or more Tregs, approximately 9.7×10⁷ or more Tregs, approximately 9.8×10⁷ or more Tregs, approximately 9.9×10⁷ or more Tregs, approximately 1.0×10⁸ or more Tregs, approximately 1.1×10⁸ or more Tregs, approximately 1.2×10⁸ or more Tregs, approximately 1.3×10⁸ or more Tregs, approximately 1.4×10⁸ or more Tregs, approximately 1.5×10⁸ or more Tregs, approximately 1.6×10⁸ or more Tregs, approximately 1.7×10⁸ or more Tregs, approximately 1.8×10⁸ or more Tregs, approximately 1.9×10⁸ or more Tregs, approximately 2.0×10⁸ or more Tregs, approximately 2.1×10⁸ or more Tregs, approximately 2.2×10⁸ or more Tregs, approximately 2.3×10⁸ or more Tregs, approximately 2.4×10⁸ or more Tregs, approximately 2.5×10⁸ or more Tregs, approximately 2.6×10⁸ or more Tregs, approximately 2.7×10⁸ or more Tregs, approximately 2.8×10⁸ or more Tregs, approximately 2.9×10⁸ or more Tregs, approximately 3.0×10⁸ or more Tregs, approximately 3.1×10⁸ or more Tregs, approximately 3.2×10⁸ or more Tregs, approximately 3.3×10⁸ or more Tregs, approximately 3.4×10⁸ or more Tregs, approximately 3.5×10⁸ or more Tregs, approximately 3.6×10⁸ or more Tregs, approximately 3.7×10⁸ or more Tregs, approximately 3.8×10⁸ or more Tregs, approximately 3.9×10⁸ or more Tregs, approximately 4.0×10⁸ or more Tregs, approximately 4.1×10⁸ or more Tregs, approximately 4.2×10⁸ or more Tregs, approximately 4.3×10⁸ or more Tregs, approximately 4.4×10⁸ or more Tregs, approximately 4.5×10⁸ or more Tregs, approximately 4.6×10⁸ or more Tregs, approximately 4.7×10⁸ or more Tregs, approximately 4.8×10⁸ or more Tregs, approximately 4.9×10⁸ or more Tregs, approximately 5.0×10⁸ or more Tregs, approximately 5.1×10⁸ or more Tregs, approximately 5.2×10⁸ or more Tregs, approximately 5.3×10⁸ or more Tregs, approximately 5.4×10⁸ or more Tregs, approximately 5.5×10⁸ or more Tregs, approximately 5.6×10⁸ or more Tregs, approximately 5.7×10⁸ or more Tregs, approximately 5.8×10⁸ or more Tregs, approximately 5.9×10⁸ or more Tregs, approximately 6.0×10⁸ or more Tregs, approximately 6.1×10⁸ or more Tregs, approximately 6.2×10⁸ or more Tregs, approximately 6.3×10⁸ or more Tregs, approximately 6.4×10⁸ or more Tregs, approximately 6.5×10⁸ or more Tregs, approximately 6.6×10⁸ or more Tregs, approximately 6.7×10⁸ or more Tregs, approximately 6.8×10⁸ or more Tregs, approximately 6.9×10⁸ or more Tregs, approximately 7.0×10⁸ or more Tregs, approximately 7.1×10⁸ or more Tregs, approximately 7.2×10⁸ or more Tregs, approximately 7.3×10⁸ or more Tregs, approximately 7.4×10⁸ or more Tregs, approximately 7.5×10⁸ or more Tregs, approximately 7.6×10⁸ or more Tregs, approximately 7.7×10⁸ or more Tregs, approximately 7.8×10⁸ or more Tregs, approximately 7.9×10⁸ or more Tregs, approximately 8.0×10⁸ or more Tregs, approximately 8.1×10⁸ or more Tregs, approximately 8.2×10⁸ or more Tregs, approximately 8.3×10⁸ or more Tregs, approximately 8.4×10⁸ or more Tregs, approximately 8.5×10⁸ or more Tregs, approximately 8.6×10⁸ or more Tregs, approximately 8.7×10⁸ or more Tregs, approximately 8.8×10⁸ or more Tregs, approximately 8.9×10⁸ or more Tregs, approximately 9.0×10⁸ or more Tregs, approximately 9.1×10⁸ or more Tregs, approximately 9.2×10⁸ or more Tregs, approximately 9.3×10⁸ or more Tregs, approximately 9.4×10⁸ or more Tregs, approximately 9.5×10⁸ or more Tregs, approximately 9.6×10⁸ or more Tregs, approximately 9.7×10⁸ or more Tregs, approximately 9.8×10⁸ or more Tregs, approximately 9.9×10⁸ or more Tregs, approximately 1.0×10⁹ or more Tregs, approximately 1.1×10⁹ or more Tregs, approximately 1.2×10⁹ or more Tregs, approximately 1.3×10⁹ or more Tregs, approximately 1.4×10⁹ or more Tregs, approximately 1.5×10⁹ or more Tregs, approximately 1.6×10⁹ or more Tregs, approximately 1.7×10⁹ or more Tregs, approximately 1.8×10⁹ or more Tregs, approximately 1.9×10⁹ or more Tregs, approximately 2.0×10⁹ or more Tregs, approximately 2.1×10⁹ or more Tregs, approximately 2.2×10⁹ or more Tregs, approximately 2.3×10⁹ or more Tregs, approximately 2.4×10⁹ or more Tregs, approximately 2.5×10⁹ or more Tregs, approximately 2.6×10⁹ or more Tregs, approximately 2.7×10⁹ or more Tregs, approximately 2.8×10⁹ or more Tregs, approximately 2.9×10⁹ or more Tregs, approximately 3.0×10⁹ or more Tregs, approximately 3.1×10⁹ or more Tregs, approximately 3.2×10⁹ or more Tregs, approximately 3.3×10⁹ or more Tregs, approximately 3.4×10⁹ or more Tregs, approximately 3.5×10⁹ or more Tregs, approximately 3.6×10⁹ or more Tregs, approximately 3.7×10⁹ or more Tregs, approximately 3.8×10⁹ or more Tregs, approximately 3.9×10⁹ or more Tregs, approximately 4.0×10⁹ or more Tregs, approximately 4.1×10⁹ or more Tregs, approximately 4.2×10⁹ or more Tregs, approximately 4.3×10⁹ or more Tregs, approximately 4.4×10⁹ or more Tregs, approximately 4.5×10⁹ or more Tregs, approximately 4.6×10⁹ or more Tregs, approximately 4.7×10⁹ or more Tregs, approximately 4.8×10⁹ or more Tregs, approximately 4.9×10⁹ or more Tregs, or approximately 5.0×10⁹ or more Tregs. In some embodiments, the Tregs are CD4⁺CD25⁺CD127^{dim}, CD3⁺CD4⁺CD25⁺, CD3⁺CD4⁺CD25⁺CD127^{dim}, CD3⁺CD4⁺CD25⁺CD127^{dim} FOXP3⁺, CD3⁺FOXP3⁺, CD3⁺CD4⁺FOXP3⁺, CD3⁺CD4⁺CD25⁺FOXP3⁺, CD3⁺CD25⁺FOXP3⁺, CD3⁺CD25⁺CD127^{dim}, CD4⁺CD25⁺, CD4⁺CD25⁺CD127^{dim} FOXP3⁺, FOXP3⁺, CD4⁺FOXP3⁺, CD4⁺CD25⁺FOXP3⁺, CD25⁺FOXP3⁺, or CD25⁺CD127^{dim}.

### Tcons

A third population of CD45⁺ cells that comprises, at least, Tcons or at least one dose of Tcons, or a population of conventional CD3⁺ T cells (Tcons) can comprise at least about 1×10⁴, at least about 2×10⁴, at least about 3×10⁴, at least about 4×10⁴, at least about 5×10⁴, at least about 6×10⁴, at least about 7×10⁴, at least about 8×10⁴, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.1×10⁶, at least about 6.2×10⁶, at least about 6.3×10⁶, at least about 6.4×10⁶, at least about 6.5×10⁶, at least about 6.6×10⁶, at least about 6.7×10⁶, at least about 6.8×10⁶, at least about 6.9×10⁶, at least about 7×10⁶, at least about 7.1×10⁶, at least about 7.2×10⁶, at least about 7.3×10⁶, at least about 7.4×10⁶, at least about 7.5×10⁶, at least about 7.6×10⁶, at least about 7.7×10⁶, at least about 7.8×10⁶, at least about 7.9×10⁶, at least about 8×10⁶, at least about 8.1×10⁶, at least about 8.2×10⁶, at least about 8.3×10⁶, at least about 8.4×10⁶, at least about 8.5×10⁶, at least about 8.6×10⁶, at least about 8.7×10⁶, at least about 8.8×10⁶, at least about 8.9×10⁶, at least about 9×10⁶, at least about 9.1×10⁶, at least about 9.2×10⁶, at least about 9.3×10⁶, at least about 9.4×10⁶, at least about 9.5×10⁶, at least about 9.6×10⁶, at least about 9.7×10⁶, at least about 9.8×10⁶, at least about 9.9×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more cells of the third population of CD45⁺ cells, the population of Tcons, and/or Tcons or doses of Tcons per kg of recipient subject's actual body weight or ideal body weight (e.g., where Tcons are CD3⁺ or CD3⁺CD127^{+/bright}).

A third population of CD45⁺ cells that comprises, at least, Tcons, or a population of conventional CD3⁺ T cells (Tcons) can comprise at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.1×10⁶, at most about 6.2×10⁶, at most about 6.3×10⁶, at most about 6.4×10⁶, at most about 6.5×10⁶, at most about 6.6×10⁶, at most about 6.7×10⁶, at most about 6.8×10⁶, at most about 6.9×10⁶, at most about 7×10⁶, at most about 7.1×10⁶, at most about 7.2×10⁶, at most about 7.3×10⁶, at most about 7.4×10⁶, at most about 7.5×10⁶, at most about 7.6×10⁶, at most about 7.7×10⁶, at most about 7.8×10⁶, at most about 7.9×10⁶, at most about 8×10⁶, at most about 8.1×10⁶, at most about 8.2×10⁶, at most about 8.3×10⁶, at most about 8.4×10⁶, at most about 8.5×10⁶, at most about 8.6×10⁶, at most about 8.7×10⁶, at most about 8.8×10⁶, at most about 8.9×10⁶, at most about 9×10⁶, at most about 9.1×10⁶, at most about 9.2×10⁶, at most about 9.3×10⁶, at most about 9.4×10⁶, at most about 9.5×10⁶, at most about 9.6×10⁶, at most about 9.7×10⁶, at most about 9.8×10⁶, at most about 9.9×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ of the third population of CD45⁺ cells, the population of Tcons, and/or Tcons or doses of Tcons per kg of recipient subject's actual body weight or ideal body weight (e.g., where Tcons are CD3⁺ or CD3⁺CD127^{+/bright}).

For example, a third population of CD45⁺ cells that comprises, at least, Tcons or at least one dose of Tcons, or a population of conventional CD3⁺ T cells (Tcons) can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ cells of the third population of CD45⁺ cells, the population of Tcons, and/or Tcons or doses of Tcons per kg of recipient subject's actual body weight or ideal body weight (e.g., where Tcons are CD3⁺ or CD3⁺CD127^{+/bright}).

In some embodiments, the third population of CD45⁺ cells comprising Tcons, or comprising at least one does of Tcons, or a population of conventional CD3⁺ T cells (Tcons) comprises from approximately 1.0×10⁵ to approximately 1.0×10¹⁰, from approximately 5.0×10⁵ to approximately 6.0×10⁹, from approximately 5.0×10⁵ to approximately 1.0×10⁸, or from approximately 1.5×10⁷ to approximately 6.0×10⁹ Tcons, e.g., in the third population of CD45⁺ cells. In some embodiments, the third population of CD45⁺ cells comprising Tcons, or comprising at least one dose of Tcons, comprises approximately 1.0×10⁵ or more Tcons, approximately 2.0×10⁵ or more Tcons, approximately 3.0×10⁵ or more Tcons, approximately 4.0×10⁵ or more Tcons, approximately 5.0×10⁵ or more Tcons, approximately 6.0×10⁵ or more Tcons, approximately 7.0×10⁵ or more Tcons, approximately 8.0×10⁵ or more Tcons, approximately 9.0×10⁵ or more Tcons, approximately 1.0×10⁶ or more Tcons, approximately 1.0×10⁶ or more Tcons, approximately 1.1×10⁶ or more Tcons, approximately 1.2×10⁶ or more Tcons, approximately 1.3×10⁶ or more Tcons, approximately 1.4×10⁶ or more Tcons, approximately 1.5×10⁶ or more Tcons, approximately 1.6×10⁶ or more Tcons, approximately 1.7×10⁶ or more Tcons, approximately 1.8×10⁶ or more Tcons, approximately 1.9×10⁶ or more Tcons, approximately 2.0×10⁶ or more Tcons, approximately 2.1×10⁶ or more Tcons, approximately 2.2×10⁶ or more Tcons, approximately 2.3×10⁶ or more Tcons, approximately 2.4×10⁶ or more Tcons, approximately 2.5×10⁶ or more Tcons, approximately 2.6×10⁶ or more Tcons, approximately 2.7×10⁶ or more Tcons, approximately 2.8×10⁶ or more Tcons, approximately 2.9×10⁶ or more Tcons, approximately 3.0×10⁶ or more Tcons, approximately 3.1×10⁶ or more Tcons, approximately 3.2×10⁶ or more Tcons, approximately 3.3×10⁶ or more Tcons, approximately 3.4×10⁶ or more Tcons, approximately 3.5×10⁶ or more Tcons, approximately 3.6×10⁶ or more Tcons, approximately 3.7×10⁶ or more Tcons, approximately 3.8×10⁶ or more Tcons, approximately 3.9×10⁶ or more Tcons, approximately 4.0×10⁶ or more Tcons, approximately 4.1×10⁶ or more Tcons, approximately 4.2×10⁶ or more Tcons, approximately 4.3×10⁶ or more Tcons, approximately 4.4×10⁶ or more Tcons, approximately 4.5×10⁶ or more Tcons, approximately 4.6×10⁶ or more Tcons, approximately 4.7×10⁶ or more Tcons, approximately 4.8×10⁶ or more Tcons, approximately 4.9×10⁶ or more Tcons, approximately 5.0×10⁶ or more Tcons, approximately 5.1×10⁶ or more Tcons, approximately 5.2×10⁶ or more Tcons, approximately 5.3×10⁶ or more Tcons, approximately 5.4×10⁶ or more Tcons, approximately 5.5×10⁶ or more Tcons, approximately 5.6×10⁶ or more Tcons, approximately 5.7×10⁶ or more Tcons, approximately 5.8×10⁶ or more Tcons, approximately 5.9×10⁶ or more Tcons, approximately 6.0×10⁶ or more Tcons, approximately 6.1×10⁶ or more Tcons, approximately 6.2×10⁶ or more Tcons, approximately 6.3×10⁶ or more Tcons, approximately 6.4×10⁶ or more Tcons, approximately 6.5×10⁶ or more Tcons, approximately 6.6×10⁶ or more Tcons, approximately 6.7×10⁶ or more Tcons, approximately 6.8×10⁶ or more Tcons, approximately 6.9×10⁶ or more Tcons, approximately 7.0×10⁶ or more Tcons, approximately 7.1×10⁶ or more Tcons, approximately 7.2×10⁶ or more Tcons, approximately 7.3×10⁶ or more Tcons, approximately 7.4×10⁶ or more Tcons, approximately 7.5×10⁶ or more Tcons, approximately 7.6×10⁶ or more Tcons, approximately 7.7×10⁶ or more Tcons, approximately 7.8×10⁶ or more Tcons, approximately 7.9×10⁶ or more Tcons, approximately 8.0×10⁶ or more Tcons, approximately 8.1×10⁶ or more Tcons, approximately 8.2×10⁶ or more Tcons, approximately 8.3×10⁶ or more Tcons, approximately 8.4×10⁶ or more Tcons, approximately 8.5×10⁶ or more Tcons, approximately 8.6×10⁶ or more Tcons, approximately 8.7×10⁶ or more Tcons, approximately 8.8×10⁶ or more Tcons, approximately 8.9×10⁶ or more Tcons, approximately 9.0×10⁶ or more Tcons, approximately 9.1×10⁶ or more Tcons, approximately 9.2×10⁶ or more Tcons, approximately 9.3×10⁶ or more Tcons, approximately 9.4×10⁶ or more Tcons, approximately 9.5×10⁶ or more Tcons, approximately 9.6×10⁶ or more Tcons, approximately 9.7×10⁶ or more Tcons, approximately 9.8×10⁶ or more Tcons, approximately 9.9×10⁶ or more Tcons, approximately 1.0×10⁷ or more Tcons, approximately 1.1×10⁷ or more Tcons, approximately 1.2×10⁷ or more Tcons, approximately 1.3×10⁷ or more Tcons, approximately 1.4×10⁷ or more Tcons, approximately 1.5×10⁷ or more Tcons, approximately 1.6×10⁷ or more Tcons, approximately 1.7×10⁷ or more Tcons, approximately 1.8×10⁷ or more Tcons, approximately 1.9×10⁷ or more Tcons, approximately 2.0×10⁷ or more Tcons, approximately 2.1×10⁷ or more Tcons, approximately 2.2×10⁷ or more Tcons, approximately 2.3×10⁷ or more Tcons, approximately 2.4×10⁷ or more Tcons, approximately 2.5×10⁷ or more Tcons, approximately 2.6×10⁷ or more Tcons, approximately 2.7×10⁷ or more Tcons, approximately 2.8×10⁷ or more Tcons, approximately 2.9×10⁷ or more Tcons, approximately 3.0×10⁷ or more Tcons, approximately 3.1×10⁷ or more Tcons, approximately 3.2×10⁷ or more Tcons, approximately 3.3×10⁷ or more Tcons, approximately 3.4×10⁷ or more Tcons, approximately 3.5×10⁷ or more Tcons, approximately 3.6×10⁷ or more Tcons, approximately 3.7×10⁷ or more Tcons, approximately 3.8×10⁷ or more Tcons, approximately 3.9×10⁷ or more Tcons, approximately 4.0×10⁷ or more Tcons, approximately 4.1×10⁷ or more Tcons, approximately 4.2×10⁷ or more Tcons, approximately 4.3×10⁷ or more Tcons, approximately 4.4×10⁷ or more Tcons, approximately 4.5×10⁷ or more Tcons, approximately 4.6×10⁷ or more Tcons, approximately 4.7×10⁷ or more Tcons, approximately 4.8×10⁷ or more Tcons, approximately 4.9×10⁷ or more Tcons, approximately 5.0×10⁷ or more Tcons, approximately 5.1×10⁷ or more Tcons, approximately 5.2×10⁷ or more Tcons, approximately 5.3×10⁷ or more Tcons, approximately 5.4×10⁷ or more Tcons, approximately 5.5×10⁷ or more Tcons, approximately 5.6×10⁷ or more Tcons, approximately 5.7×10⁷ or more Tcons, approximately 5.8×10⁷ or more Tcons, approximately 5.9×10⁷ or more Tcons, approximately 6.0×10⁷ or more Tcons, approximately 6.1×10⁷ or more Tcons, approximately 6.2×10⁷ or more Tcons, approximately 6.3×10⁷ or more Tcons, approximately 6.4×10⁷ or more Tcons, approximately 6.5×10⁷ or more Tcons, approximately 6.6×10⁷ or more Tcons, approximately 6.7×10⁷ or more Tcons, approximately 6.8×10⁷ or more Tcons, approximately 6.9×10⁷ or more Tcons, approximately 7.0×10⁷ or more Tcons, approximately 7.1×10⁷ or more Tcons, approximately 7.2×10⁷ or more Tcons, approximately 7.3×10⁷ or more Tcons, approximately 7.4×10⁷ or more Tcons, approximately 7.5×10⁷ or more Tcons, approximately 7.6×10⁷ or more Tcons, approximately 7.7×10⁷ or more Tcons, approximately 7.8×10⁷ or more Tcons, approximately 7.9×10⁷ or more Tcons, approximately 8.0×10⁷ or more Tcons, approximately 8.1×10⁷ or more Tcons, approximately 8.2×10⁷ or more Tcons, approximately 8.3×10⁷ or more Tcons, approximately 8.4×10⁷ or more Tcons, approximately 8.5×10⁷ or more Tcons, approximately 8.6×10⁷ or more Tcons, approximately 8.7×10⁷ or more Tcons, approximately 8.8×10⁷ or more Tcons, approximately 8.9×10⁷ or more Tcons, approximately 9.0×10⁷ or more Tcons, approximately 9.1×10⁷ or more Tcons, approximately 9.2×10⁷ or more Tcons, approximately 9.3×10⁷ or more Tcons, approximately 9.4×10⁷ or more Tcons, approximately 9.5×10⁷ or more Tcons, approximately 9.6×10⁷ or more Tcons, approximately 9.7×10⁷ or more Tcons, approximately 9.8×10⁷ or more Tcons, approximately 9.9×10⁷ or more Tcons, approximately 1.0×10⁸ or more Tcons, approximately 1.1×10⁸ or more Tcons, approximately 1.2×10⁸ or more Tcons, approximately 1.3×10⁸ or more Tcons, approximately 1.4×10⁸ or more Tcons, approximately 1.5×10⁸ or more Tcons, approximately 1.6×10⁸ or more Tcons, approximately 1.7×10⁸ or more Tcons, approximately 1.8×10⁸ or more Tcons, approximately 1.9×10⁸ or more Tcons, approximately 2.0×10⁸ or more Tcons, approximately 2.1×10⁸ or more Tcons, approximately 2.2×10⁸ or more Tcons, approximately 2.3×10⁸ or more Tcons, approximately 2.4×10⁸ or more Tcons, approximately 2.5×10⁸ or more Tcons, approximately 2.6×10⁸ or more Tcons, approximately 2.7×10⁸ or more Tcons, approximately 2.8×10⁸ or more Tcons, approximately 2.9×10⁸ or more Tcons, approximately 3.0×10⁸ or more Tcons, approximately 3.1×10⁸ or more Tcons, approximately 3.2×10⁸ or more Tcons, approximately 3.3×10⁸ or more Tcons, approximately 3.4×10⁸ or more Tcons, approximately 3.5×10⁸ or more Tcons, approximately 3.6×10⁸ or more Tcons, approximately 3.7×10⁸ or more Tcons, approximately 3.8×10⁸ or more Tcons, approximately 3.9×10⁸ or more Tcons, approximately 4.0×10⁸ or more Tcons, approximately 4.1×10⁸ or more Tcons, approximately 4.2×10⁸ or more Tcons, approximately 4.3×10⁸ or more Tcons, approximately 4.4×10⁸ or more Tcons, approximately 4.5×10⁸ or more Tcons, approximately 4.6×10⁸ or more Tcons, approximately 4.7×10⁸ or more Tcons, approximately 4.8×10⁸ or more Tcons, approximately 4.9×10⁸ or more Tcons, approximately 5.0×10⁸ or more Tcons, approximately 5.1×10⁸ or more Tcons, approximately 5.2×10⁸ or more Tcons, approximately 5.3×10⁸ or more Tcons, approximately 5.4×10⁸ or more Tcons, approximately 5.5×10⁸ or more Tcons, approximately 5.6×10⁸ or more Tcons, approximately 5.7×10⁸ or more Tcons, approximately 5.8×10⁸ or more Tcons, approximately 5.9×10⁸ or more Tcons, approximately 6.0×10⁸ or more Tcons, approximately 6.1×10⁸ or more Tcons, approximately 6.2×10⁸ or more Tcons, approximately 6.3×10⁸ or more Tcons, approximately 6.4×10⁸ or more Tcons, approximately 6.5×10⁸ or more Tcons, approximately 6.6×10⁸ or more Tcons, approximately 6.7×10⁸ or more Tcons, approximately 6.8×10⁸ or more Tcons, approximately 6.9×10⁸ or more Tcons, approximately 7.0×10⁸ or more Tcons, approximately 7.1×10⁸ or more Tcons, approximately 7.2×10⁸ or more Tcons, approximately 7.3×10⁸ or more Tcons, approximately 7.4×10⁸ or more Tcons, approximately 7.5×10⁸ or more Tcons, approximately 7.6×10⁸ or more Tcons, approximately 7.7×10⁸ or more Tcons, approximately 7.8×10⁸ or more Tcons, approximately 7.9×10⁸ or more Tcons, approximately 8.0×10⁸ or more Tcons, approximately 8.1×10⁸ or more Tcons, approximately 8.2×10⁸ or more Tcons, approximately 8.3×10⁸ or more Tcons, approximately 8.4×10⁸ or more Tcons, approximately 8.5×10⁸ or more Tcons, approximately 8.6×10⁸ or more Tcons, approximately 8.7×10⁸ or more Tcons, approximately 8.8×10⁸ or more Tcons, approximately 8.9×10⁸ or more Tcons, approximately 9.0×10⁸ or more Tcons, approximately 9.1×10⁸ or more Tcons, approximately 9.2×10⁸ or more Tcons, approximately 9.3×10⁸ or more Tcons, approximately 9.4×10⁸ or more Tcons, approximately 9.5×10⁸ or more Tcons, approximately 9.6×10⁸ or more Tcons, approximately 9.7×10⁸ or more Tcons, approximately 9.8×10⁸ or more Tcons, approximately 9.9×10⁸ or more Tcons, approximately 1.0×10⁹ or more Tcons, approximately 1.1×10⁹ or more Tcons, approximately 1.2×10⁹ or more Tcons, approximately 1.3×10⁹ or more Tcons, approximately 1.4×10⁹ or more Tcons, approximately 1.5×10⁹ or more Tcons, approximately 1.6×10⁹ or more Tcons, approximately 1.7×10⁹ or more Tcons, approximately 1.8×10⁹ or more Tcons, approximately 1.9×10⁹ or more Tcons, approximately 2.0×10⁹ or more Tcons, approximately 2.1×10⁹ or more Tcons, approximately 2.2×10⁹ or more Tcons, approximately 2.3×10⁹ or more Tcons, approximately 2.4×10⁹ or more Tcons, approximately 2.5×10⁹ or more Tcons, approximately 2.6×10⁹ or more Tcons, approximately 2.7×10⁹ or more Tcons, approximately 2.8×10⁹ or more Tcons, approximately 2.9×10⁹ or more Tcons, approximately 3.0×10⁹ or more Tcons, approximately 3.1×10⁹ or more Tcons, approximately 3.2×10⁹ or more Tcons, approximately 3.3×10⁹ or more Tcons, approximately 3.4×10⁹ or more Tcons, approximately 3.5×10⁹ or more Tcons, approximately 3.6×10⁹ or more Tcons, approximately 3.7×10⁹ or more Tcons, approximately 3.8×10⁹ or more Tcons, approximately 3.9×10⁹ or more Tcons, approximately 4.0×10⁹ or more Tcons, approximately 4.1×10⁹ or more Tcons, approximately 4.2×10⁹ or more Tcons, approximately 4.3×10⁹ or more Tcons, approximately 4.4×10⁹ or more Tcons, approximately 4.5×10⁹ or more Tcons, approximately 4.6×10⁹ or more Tcons, approximately 4.7×10⁹ or more Tcons, approximately 4.8×10⁹ or more Tcons, approximately 4.9×10⁹ or more Tcons, approximately 5.0×10⁹ or more Tcons, approximately 5.1×10⁹ or more Tcons, approximately 5.2×10⁹ or more Tcons, approximately 5.3×10⁹ or more Tcons, approximately 5.4×10⁹ or more Tcons, approximately 5.5×10⁹ or more Tcons, approximately 5.6×10⁹ or more Tcons, approximately 5.7×10⁹ or more Tcons, approximately 5.8×10⁹ or more Tcons, approximately 5.9×10⁹ or more Tcons, approximately 6.0×10⁹ or more Tcons, approximately 6.1×10⁹ or more Tcons, approximately 6.2×10⁹ or more Tcons, approximately 6.3×10⁹ or more Tcons, approximately 6.4×10⁹ or more Tcons, approximately 6.5×10⁹ or more Tcons, approximately 6.6×10⁹ or more Tcons, approximately 6.7×10⁹ or more Tcons, approximately 6.8×10⁹ or more Tcons, approximately 6.9×10⁹ or more Tcons, approximately 7.0×10⁹ or more Tcons, approximately 7.1×10⁹ or more Tcons, approximately 7.2×10⁹ or more Tcons, approximately 7.3×10⁹ or more Tcons, approximately 7.4×10⁹ or more Tcons, approximately 7.5×10⁹ or more Tcons, approximately 7.6×10⁹ or more Tcons, approximately 7.7×10⁹ or more Tcons, approximately 7.8×10⁹ or more Tcons, approximately 7.9×10⁹ or more Tcons, approximately 8.0×10⁹ or more Tcons, approximately 8.1×10⁹ or more Tcons, approximately 8.2×10⁹ or more Tcons, approximately 8.3×10⁹ or more Tcons, approximately 8.4×10⁹ or more Tcons, approximately 8.5×10⁹ or more Tcons, approximately 8.6×10⁹ or more Tcons, approximately 8.7×10⁹ or more Tcons, approximately 8.8×10⁹ or more Tcons, approximately 8.9×10⁹ or more Tcons, approximately 9.0×10⁹ or more Tcons, approximately 9.1×10⁹ or more Tcons, approximately 9.2×10⁹ or more Tcons, approximately 9.3×10⁹ or more Tcons, approximately 9.4×10⁹ or more Tcons, approximately 9.5×10⁹ or more Tcons, approximately 9.6×10⁹ or more Tcons, approximately 9.7×10⁹ or more Tcons, approximately 9.8×10⁹ or more Tcons, approximately 9.9×10⁹ or more Tcons, or approximately 1.0×10¹⁰ or more Tcons. In some embodiments, the Tcons are CD3⁺ or CD3⁺CD127^{+/bright}.

In a third population of CD45⁺ cells or in a population of conventional CD3⁺ T cells (Tcons), the ratio of Tcons: Tregs administered to a subject can be, for example, about 1:100, 1:50, 1:25, 1:20, 1:15, 1:10, 1:9, 1:8, 1:7, 1:6, 1:5, 1:4, 1:3, 1:2.5, 1:2, 1.5:2, 1:1.5, 1:1, 1.5:1, 2:1, 2:1.5, 2.5:1, 3:1, 4:1, 5:1, 6:1, 7:1, 8:1, 9:1, 10:1, 15:1, 20:1, 25:1, 50:1, or 100:1.

Cells of the third population of CD45⁺ cells that comprises, at least, Tcons or a population of conventional CD3⁺ T cells (Tcons) can be cryopreserved for any amount of time. Cells of the third population of CD45⁺ cells or of the population of conventional CD3⁺ T cells (Tcons) may be cryopreserved for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 at least about 14 hours, at least about 16 hours, at least about 18 hours, at least about 20 hours, at least about 22 hours, at least about 24 hours, at least about 30 hours, at least about 36 hours at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to thawing and administration to a subject.

In some embodiments, cells of the third population of CD45⁺ cells that comprises, at least, Tcons or cells of a population of conventional CD3⁺ T cells (Tcons) are cryopreserved for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 at most about 14 hours, at most about 16 hours, at most about 18 hours, at most about 20 hours, at most about 22 hours, at most about 24 hours, at most about 30 hours, at most about 36 hours at most about 48 hours, at most about 50 hours, at most about 55 hours, at most about 60 hours, at most about 61 hours, at most about 62 hours, at most about 65 hours, at most about 70 hours, at most about 72 hours, at most about 80 hours, at most about 90 hours, at most about 96 hours, at most about 120 hours, at most about 150 hours, at most about 200 hours, or at most about 300 hours prior to thawing and administration to a subject.

In some embodiments, cells of the third population of CD45⁺ cells or cells of the population of conventional CD3⁺ T cells (Tcons) are cryopreserved for at least about 1 day, at least about 2 days, at least about 3 days, at least about 4 days, at least about 5 days, at least about 6 days, at least about 7 days, at least about 10 days, at least about 14 days, at least about 21 days, at least about 28 days, at least about 50 days, at least about 60 days, or at least about 96 days, or more prior to thawing and administration to a subject.

In some embodiments, cells of the third population of CD45⁺ cells or cells of the population of conventional CD3⁺ T cells (Tcons) are cryopreserved for at most about 1 day, at most about 2 days, at most about 3 days, at most about 4 days, at most about 5 days, at most about 6 days, at most about 7 days, at most about 10 days, at most about 14 days, at most about 21 days, at most about 28 days, at most about 50 days, at most about 60 days, or at most about 96 days prior to thawing and administration to a subject.

iNKTs

A cell population that comprises a population of iNKTs can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more iNKTs per kg of recipient subject's actual body weight or ideal body weight (e.g., where iNKTs are CD3⁺Vα24Jα18⁺).

A cell population that comprises a population of iNKTs can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, at most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ iNKTs per kg of recipient subject's actual body weight or ideal body weight (e.g., where iNKTs are CD3⁺Vα24Jα18⁺).

For example, a cell population that comprises a population of iNKTs can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ iNKTs per kg of recipient subject's actual body weight or ideal body weight (e.g., where iNKTs are CD3⁺Vα24Jα18⁺).

A population of iNKTs of the disclosure can have a defined level of purity for iNKT cells. For example, a population of iNKTs of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more iNKT cells as a percentage of total cells, as a percentage of nucleated cells, or as a percentage of CD45⁺ cells (e.g., where iNKTs are CD3⁺Vα24Jα18⁺).

A population of iNKTs of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, iNKTs as a percentage of total cells, nucleated cells, or CD45⁺ cells (e.g., where iNKTs are CD3⁺Vα24Jα18⁺).

A population of iNKTs of the disclosure can have a defined level of contaminating non-iNKT cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-iNKT cells per kg of recipient subject's actual body weight or ideal body weight are present in a population of iNKTs of the disclosure, (e.g., where non-iNKT cells are Vα24Jα18⁻).

In some embodiments, a population of iNKTs of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-iNKT cells, (e.g., where non-iNKT cells are Vα24Jα18⁻).

### Tmems

In some embodiments, a population of cells of the present disclosure comprising Tmem, or comprising at least one dose of Tmem, comprises from about 1×10⁴ to about 1×10⁹ Tmem per kilogram of actual body weight or ideal body weight of a human subject receiving a treatment or cellular therapy product of the present disclosure. In some embodiments, the population of cells comprising Tmem, or comprising at least one dose of Tmem, comprises from about 1×10⁴ to about 1×10⁹ Tmem per kilogram of IBW of the human subject (e.g., where Tmem are CD3+CD45RA−CD45RO+).

A population of cells of the present disclosure comprising Tmem, or comprising at least one dose of Tmem, can comprise at least about 1×10⁴, at least about 2×10⁴, at least about 3×10⁴, at least about 4×10⁴, at least about 5×10⁴, at least about 6×10⁴, at least about 7×10⁴, at least about 8×10⁴, at least about 9×10⁴, at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.1×10⁶, at least about 6.2×10⁶, at least about 6.3×10⁶, at least about 6.4×10⁶, at least about 6.5×10⁶, at least about 6.6×10⁶, at least about 6.7×10⁶, at least about 6.8×10⁶, at least about 6.9×10⁶, at least about 7×10⁶, at least about 7.1×10⁶, at least about 7.2×10⁶, at least about 7.3×10⁶, at least about 7.4×10⁶, at least about 7.5×10⁶, at least about 7.6×10⁶, at least about 7.7×10⁶, at least about 7.8×10⁶, at least about 7.9×10⁶, at least about 8×10⁶, at least about 8.1×10⁶, at least about 8.2×10⁶, at least about 8.3×10⁶, at least about 8.4×10⁶, at least about 8.5×10⁶, at least about 8.6×10⁶, at least about 8.7×10⁶, at least about 8.8×10⁶, at least about 8.9×10⁶, at least about 9×10⁶, at least about 9.1×10⁶, at least about 9.2×10⁶, at least about 9.3×10⁶, at least about 9.4×10⁶, at least about 9.5×10⁶, at least about 9.6×10⁶, at least about 9.7×10⁶, at least about 9.8×10⁶, at least about 9.9×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more Tmem per kilogram (kg) of the human subject's actual body weight or (adjusted) ideal body weight (e.g., where Tmem are CD3+CD45RA−CD45RO+).

A population of cells of the present disclosure comprising Tmem, or comprising at least one dose of Tmem, can comprise at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, at most about 1×10⁵, at most about 2×10⁵, at most about 3×10⁵, at most about 4×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, at most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, at most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.1×10⁶, at most about 6.2×10⁶, at most about 6.3×10⁶, at most about 6.4×10⁶, at most about 6.5×10⁶, at most about 6.6×10⁶, at most about 6.7×10⁶, at most about 6.8×10⁶, at most about 6.9×10⁶, at most about 7×10⁶, at most about 7.1×10⁶, at most about 7.2×10⁶, at most about 7.3×10⁶, at most about 7.4×10⁶, at most about 7.5×10⁶, at most about 7.6×10⁶, at most about 7.7×10⁶, at most about 7.8×10⁶, at most about 7.9×10⁶, at most about 8×10⁶, at most about 8.1×10⁶, at most about 8.2×10⁶, at most about 8.3×10⁶, at most about 8.4×10⁶, at most about 8.5×10⁶, at most about 8.6×10⁶, at most about 8.7×10⁶, at most about 8.8×10⁶, at most about 8.9×10⁶, at most about 9×10⁶, at most about 9.1×10⁶, at most about 9.2×10⁶, at most about 9.3×10⁶, at most about 9.4×10⁶, at most about 9.5×10⁶, at most about 9.6×10⁶, at most about 9.7×10⁶, at most about 9.8×10⁶, at most about 9.9×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ Tmem per kg of the human subject's actual body weight or (adjusted) ideal body weight (e.g., where Tmem are CD3+CD45RA−CD45RO+).

For example, a population of cells of the present disclosure comprising Tmem, or comprising at least one dose of Tmem, can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ Tmem per kg of the human subject's actual body weight or (adjusted) ideal body weight (e.g., where Tmem are CD3+CD45RA−CD45RO+).

In some embodiments, a population of cells of the present disclosure comprising Tmem, or comprising at least one dose of Tmem, can comprise from approximately 1.0×10⁵ to approximately 5.0×10¹⁰, from approximately 5.0×10⁵ to approximately 1.5×10¹⁰, from approximately 5.0×10⁵ to approximately 5.0×10⁸, or from approximately 1.5×10⁷ to approximately 1.5×10¹⁰ Tmem. In some embodiments, a population of Tmem, or a dose of Tmem, can comprise approximately 1.0×10⁵ or more Tmem, approximately 2.0×10⁵ or more Tmem, approximately 3.0×10⁵ or more Tmem, approximately 4.0×10⁵ or more Tmem, approximately 5.0×10⁵ or more Tmem, approximately 6.0×10⁵ or more Tmem, approximately 7.0×10⁵ or more Tmem, approximately 8.0×10⁵ or more Tmem, approximately 9.0×10⁵ or more Tmem, approximately 1.0×10⁶ or more Tmem, approximately 1.0×10⁶ or more Tmem, approximately 1.1×10⁶ or more Tmem, approximately 1.2×10⁶ or more Tmem, approximately 1.3×10⁶ or more Tmem, approximately 1.4×10⁶ or more Tmem, approximately 1.5×10⁶ or more Tmem, approximately 1.6×10⁶ or more Tmem, approximately 1.7×10⁶ or more Tmem, approximately 1.8×10⁶ or more Tmem, approximately 1.9×10⁶ or more Tmem, approximately 2.0×10⁶ or more Tmem, approximately 2.1×10⁶ or more Tmem, approximately 2.2×10⁶ or more Tmem, approximately 2.3×10⁶ or more Tmem, approximately 2.4×10⁶ or more Tmem, approximately 2.5×10⁶ or more Tmem, approximately 2.6×10⁶ or more Tmem, approximately 2.7×10⁶ or more Tmem, approximately 2.8×10⁶ or more Tmem, approximately 2.9×10⁶ or more Tmem, approximately 3.0×10⁶ or more Tmem, approximately 3.1×10⁶ or more Tmem, approximately 3.2×10⁶ or more Tmem, approximately 3.3×10⁶ or more Tmem, approximately 3.4×10⁶ or more Tmem, approximately 3.5×10⁶ or more Tmem, approximately 3.6×10⁶ or more Tmem, approximately 3.7×10⁶ or more Tmem, approximately 3.8×10⁶ or more Tmem, approximately 3.9×10⁶ or more Tmem, approximately 4.0×10⁶ or more Tmem, approximately 4.1×10⁶ or more Tmem, approximately 4.2×10⁶ or more Tmem, approximately 4.3×10⁶ or more Tmem, approximately 4.4×10⁶ or more Tmem, approximately 4.5×10⁶ or more Tmem, approximately 4.6×10⁶ or more Tmem, approximately 4.7×10⁶ or more Tmem, approximately 4.8×10⁶ or more Tmem, approximately 4.9×10⁶ or more Tmem, approximately 5.0×10⁶ or more Tmem, approximately 5.1×10⁶ or more Tmem, approximately 5.2×10⁶ or more Tmem, approximately 5.3×10⁶ or more Tmem, approximately 5.4×10⁶ or more Tmem, approximately 5.5×10⁶ or more Tmem, approximately 5.6×10⁶ or more Tmem, approximately 5.7×10⁶ or more Tmem, approximately 5.8×10⁶ or more Tmem, approximately 5.9×10⁶ or more Tmem, approximately 6.0×10⁶ or more Tmem, approximately 6.1×10⁶ or more Tmem, approximately 6.2×10⁶ or more Tmem, approximately 6.3×10⁶ or more Tmem, approximately 6.4×10⁶ or more Tmem, approximately 6.5×10⁶ or more Tmem, approximately 6.6×10⁶ or more Tmem, approximately 6.7×10⁶ or more Tmem, approximately 6.8×10⁶ or more Tmem, approximately 6.9×10⁶ or more Tmem, approximately 7.0×10⁶ or more Tmem, approximately 7.1×10⁶ or more Tmem, approximately 7.2×10⁶ or more Tmem, approximately 7.3×10⁶ or more Tmem, approximately 7.4×10⁶ or more Tmem, approximately 7.5×10⁶ or more Tmem, approximately 7.6×10⁶ or more Tmem, approximately 7.7×10⁶ or more Tmem, approximately 7.8×10⁶ or more Tmem, approximately 7.9×10⁶ or more Tmem, approximately 8.0×10⁶ or more Tmem, approximately 8.1×10⁶ or more Tmem, approximately 8.2×10⁶ or more Tmem, approximately 8.3×10⁶ or more Tmem, approximately 8.4×10⁶ or more Tmem, approximately 8.5×10⁶ or more Tmem, approximately 8.6×10⁶ or more Tmem, approximately 8.7×10⁶ or more Tmem, approximately 8.8×10⁶ or more Tmem, approximately 8.9×10⁶ or more Tmem, approximately 9.0×10⁶ or more Tmem, approximately 9.1×10⁶ or more Tmem, approximately 9.2×10⁶ or more Tmem, approximately 9.3×10⁶ or more Tmem, approximately 9.4×10⁶ or more Tmem, approximately 9.5×10⁶ or more Tmem, approximately 9.6×10⁶ or more Tmem, approximately 9.7×10⁶ or more Tmem, approximately 9.8×10⁶ or more Tmem, approximately 9.9×10⁶ or more Tmem, approximately 1.0×10⁷ or more Tmem, approximately 1.1×10⁷ or more Tmem, approximately 1.2×10⁷ or more Tmem, approximately 1.3×10⁷ or more Tmem, approximately 1.4×10⁷ or more Tmem, approximately 1.5×10⁷ or more Tmem, approximately 1.6×10⁷ or more Tmem, approximately 1.7×10⁷ or more Tmem, approximately 1.8×10⁷ or more Tmem, approximately 1.9×10⁷ or more Tmem, approximately 2.0×10⁷ or more Tmem, approximately 2.1×10⁷ or more Tmem, approximately 2.2×10⁷ or more Tmem, approximately 2.3×10⁷ or more Tmem, approximately 2.4×10⁷ or more Tmem, approximately 2.5×10⁷ or more Tmem, approximately 2.6×10⁷ or more Tmem, approximately 2.7×10⁷ or more Tmem, approximately 2.8×10⁷ or more Tmem, approximately 2.9×10⁷ or more Tmem, approximately 3.0×10⁷ or more Tmem, approximately 3.1×10⁷ or more Tmem, approximately 3.2×10⁷ or more Tmem, approximately 3.3×10⁷ or more Tmem, approximately 3.4×10⁷ or more Tmem, approximately 3.5×10⁷ or more Tmem, approximately 3.6×10⁷ or more Tmem, approximately 3.7×10⁷ or more Tmem, approximately 3.8×10⁷ or more Tmem, approximately 3.9×10⁷ or more Tmem, approximately 4.0×10⁷ or more Tmem, approximately 4.1×10⁷ or more Tmem, approximately 4.2×10⁷ or more Tmem, approximately 4.3×10⁷ or more Tmem, approximately 4.4×10⁷ or more Tmem, approximately 4.5×10⁷ or more Tmem, approximately 4.6×10⁷ or more Tmem, approximately 4.7×10⁷ or more Tmem, approximately 4.8×10⁷ or more Tmem, approximately 4.9×10⁷ or more Tmem, approximately 5.0×10⁷ or more Tmem, approximately 5.1×10⁷ or more Tmem, approximately 5.2×10⁷ or more Tmem, approximately 5.3×10⁷ or more Tmem, approximately 5.4×10⁷ or more Tmem, approximately 5.5×10⁷ or more Tmem, approximately 5.6×10⁷ or more Tmem, approximately 5.7×10⁷ or more Tmem, approximately 5.8×10⁷ or more Tmem, approximately 5.9×10⁷ or more Tmem, approximately 6.0×10⁷ or more Tmem, approximately 6.1×10⁷ or more Tmem, approximately 6.2×10⁷ or more Tmem, approximately 6.3×10⁷ or more Tmem, approximately 6.4×10⁷ or more Tmem, approximately 6.5×10⁷ or more Tmem, approximately 6.6×10⁷ or more Tmem, approximately 6.7×10⁷ or more Tmem, approximately 6.8×10⁷ or more Tmem, approximately 6.9×10⁷ or more Tmem, approximately 7.0×10⁷ or more Tmem, approximately 7.1×10⁷ or more Tmem, approximately 7.2×10⁷ or more Tmem, approximately 7.3×10⁷ or more Tmem, approximately 7.4×10⁷ or more Tmem, approximately 7.5×10⁷ or more Tmem, approximately 7.6×10⁷ or more Tmem, approximately 7.7×10⁷ or more Tmem, approximately 7.8×10⁷ or more Tmem, approximately 7.9×10⁷ or more Tmem, approximately 8.0×10⁷ or more Tmem, approximately 8.1×10⁷ or more Tmem, approximately 8.2×10⁷ or more Tmem, approximately 8.3×10⁷ or more Tmem, approximately 8.4×10⁷ or more Tmem, approximately 8.5×10⁷ or more Tmem, approximately 8.6×10⁷ or more Tmem, approximately 8.7×10⁷ or more Tmem, approximately 8.8×10⁷ or more Tmem, approximately 8.9×10⁷ or more Tmem, approximately 9.0×10⁷ or more Tmem, approximately 9.1×10⁷ or more Tmem, approximately 9.2×10⁷ or more Tmem, approximately 9.3×10⁷ or more Tmem, approximately 9.4×10⁷ or more Tmem, approximately 9.5×10⁷ or more Tmem, approximately 9.6×10⁷ or more Tmem, approximately 9.7×10⁷ or more Tmem, approximately 9.8×10⁷ or more Tmem, approximately 9.9×10⁷ or more Tmem, approximately 1.0×10⁸ or more Tmem, approximately 1.1×10⁸ or more Tmem, approximately 1.2×10⁸ or more Tmem, approximately 1.3×10⁸ or more Tmem, approximately 1.4×10⁸ or more Tmem, approximately 1.5×10⁸ or more Tmem, approximately 1.6×10⁸ or more Tmem, approximately 1.7×10⁸ or more Tmem, approximately 1.8×10⁸ or more Tmem, approximately 1.9×10⁸ or more Tmem, approximately 2.0×10⁸ or more Tmem, approximately 2.1×10⁸ or more Tmem, approximately 2.2×10⁸ or more Tmem, approximately 2.3×10⁸ or more Tmem, approximately 2.4×10⁸ or more Tmem, approximately 2.5×10⁸ or more Tmem, approximately 2.6×10⁸ or more Tmem, approximately 2.7×10⁸ or more Tmem, approximately 2.8×10⁸ or more Tmem, approximately 2.9×10⁸ or more Tmem, approximately 3.0×10⁸ or more Tmem, approximately 3.1×10⁸ or more Tmem, approximately 3.2×10⁸ or more Tmem, approximately 3.3×10⁸ or more Tmem, approximately 3.4×10⁸ or more Tmem, approximately 3.5×10⁸ or more Tmem, approximately 3.6×10⁸ or more Tmem, approximately 3.7×10⁸ or more Tmem, approximately 3.8×10⁸ or more Tmem, approximately 3.9×10⁸ or more Tmem, approximately 4.0×10⁸ or more Tmem, approximately 4.1×10⁸ or more Tmem, approximately 4.2×10⁸ or more Tmem, approximately 4.3×10⁸ or more Tmem, approximately 4.4×10⁸ or more Tmem, approximately 4.5×10⁸ or more Tmem, approximately 4.6×10⁸ or more Tmem, approximately 4.7×10⁸ or more Tmem, approximately 4.8×10⁸ or more Tmem, approximately 4.9×10⁸ or more Tmem, approximately 5.0×10⁸ or more Tmem, approximately 5.1×10⁸ or more Tmem, approximately 5.2×10⁸ or more Tmem, approximately 5.3×10⁸ or more Tmem, approximately 5.4×10⁸ or more Tmem, approximately 5.5×10⁸ or more Tmem, approximately 5.6×10⁸ or more Tmem, approximately 5.7×10⁸ or more Tmem, approximately 5.8×10⁸ or more Tmem, approximately 5.9×10⁸ or more Tmem, approximately 6.0×10⁸ or more Tmem, approximately 6.1×10⁸ or more Tmem, approximately 6.2×10⁸ or more Tmem, approximately 6.3×10⁸ or more Tmem, approximately 6.4×10⁸ or more Tmem, approximately 6.5×10⁸ or more Tmem, approximately 6.6×10⁸ or more Tmem, approximately 6.7×10⁸ or more Tmem, approximately 6.8×10⁸ or more Tmem, approximately 6.9×10⁸ or more Tmem, approximately 7.0×10⁸ or more Tmem, approximately 7.1×10⁸ or more Tmem, approximately 7.2×10⁸ or more Tmem, approximately 7.3×10⁸ or more Tmem, approximately 7.4×10⁸ or more Tmem, approximately 7.5×10⁸ or more Tmem, approximately 7.6×10⁸ or more Tmem, approximately 7.7×10⁸ or more Tmem, approximately 7.8×10⁸ or more Tmem, approximately 7.9×10⁸ or more Tmem, approximately 8.0×10⁸ or more Tmem, approximately 8.1×10⁸ or more Tmem, approximately 8.2×10⁸ or more Tmem, approximately 8.3×10⁸ or more Tmem, approximately 8.4×10⁸ or more Tmem, approximately 8.5×10⁸ or more Tmem, approximately 8.6×10⁸ or more Tmem, approximately 8.7×10⁸ or more Tmem, approximately 8.8×10⁸ or more Tmem, approximately 8.9×10⁸ or more Tmem, approximately 9.0×10⁸ or more Tmem, approximately 9.1×10⁸ or more Tmem, approximately 9.2×10⁸ or more Tmem, approximately 9.3×10⁸ or more Tmem, approximately 9.4×10⁸ or more Tmem, approximately 9.5×10⁸ or more Tmem, approximately 9.6×10⁸ or more Tmem, approximately 9.7×10⁸ or more Tmem, approximately 9.8×10⁸ or more Tmem, approximately 9.9×10⁸ or more Tmem, approximately 1.0×10⁹ or more Tmem, approximately 1.1×10⁹ or more Tmem, approximately 1.2×10⁹ or more Tmem, approximately 1.3×10⁹ or more Tmem, approximately 1.4×10⁹ or more Tmem, approximately 1.5×10⁹ or more Tmem, approximately 1.6×10⁹ or more Tmem, approximately 1.7×10⁹ or more Tmem, approximately 1.8×10⁹ or more Tmem, approximately 1.9×10⁹ or more Tmem, approximately 2.0×10⁹ or more Tmem, approximately 2.1×10⁹ or more Tmem, approximately 2.2×10⁹ or more Tmem, approximately 2.3×10⁹ or more Tmem, approximately 2.4×10⁹ or more Tmem, approximately 2.5×10⁹ or more Tmem, approximately 2.6×10⁹ or more Tmem, approximately 2.7×10⁹ or more Tmem, approximately 2.8×10⁹ or more Tmem, approximately 2.9×10⁹ or more Tmem, approximately 3.0×10⁹ or more Tmem, approximately 3.1×10⁹ or more Tmem, approximately 3.2×10⁹ or more Tmem, approximately 3.3×10⁹ or more Tmem, approximately 3.4×10⁹ or more Tmem, approximately 3.5×10⁹ or more Tmem, approximately 3.6×10⁹ or more Tmem, approximately 3.7×10⁹ or more Tmem, approximately 3.8×10⁹ or more Tmem, approximately 3.9×10⁹ or more Tmem, approximately 4.0×10⁹ or more Tmem, approximately 4.1×10⁹ or more Tmem, approximately 4.2×10⁹ or more Tmem, approximately 4.3×10⁹ or more Tmem, approximately 4.4×10⁹ or more Tmem, approximately 4.5×10⁹ or more Tmem, approximately 4.6×10⁹ or more Tmem, approximately 4.7×10⁹ or more Tmem, approximately 4.8×10⁹ or more Tmem, approximately 4.9×10⁹ or more Tmem, approximately 5.0×10⁹ or more Tmem, approximately 5.1×10⁹ or more Tmem, approximately 5.2×10⁹ or more Tmem, approximately 5.3×10⁹ or more Tmem, approximately 5.4×10⁹ or more Tmem, approximately 5.5×10⁹ or more Tmem, approximately 5.6×10⁹ or more Tmem, approximately 5.7×10⁹ or more Tmem, approximately 5.8×10⁹ or more Tmem, approximately 5.9×10⁹ or more Tmem, approximately 6.0×10⁹ or more Tmem, approximately 6.1×10⁹ or more Tmem, approximately 6.2×10⁹ or more Tmem, approximately 6.3×10⁹ or more Tmem, approximately 6.4×10⁹ or more Tmem, approximately 6.5×10⁹ or more Tmem, approximately 6.6×10⁹ or more Tmem, approximately 6.7×10⁹ or more Tmem, approximately 6.8×10⁹ or more Tmem, approximately 6.9×10⁹ or more Tmem, approximately 7.0×10⁹ or more Tmem, approximately 7.1×10⁹ or more Tmem, approximately 7.2×10⁹ or more Tmem, approximately 7.3×10⁹ or more Tmem, approximately 7.4×10⁹ or more Tmem, approximately 7.5×10⁹ or more Tmem, approximately 7.6×10⁹ or more Tmem, approximately 7.7×10⁹ or more Tmem, approximately 7.8×10⁹ or more Tmem, approximately 7.9×10⁹ or more Tmem, approximately 8.0×10⁹ or more Tmem, approximately 8.1×10⁹ or more Tmem, approximately 8.2×10⁹ or more Tmem, approximately 8.3×10⁹ or more Tmem, approximately 8.4×10⁹ or more Tmem, approximately 8.5×10⁹ or more Tmem, approximately 8.6×10⁹ or more Tmem, approximately 8.7×10⁹ or more Tmem, approximately 8.8×10⁹ or more Tmem, approximately 8.9×10⁹ or more Tmem, approximately 9.0×10⁹ or more Tmem, approximately 9.1×10⁹ or more Tmem, approximately 9.2×10⁹ or more Tmem, approximately 9.3×10⁹ or more Tmem, approximately 9.4×10⁹ or more Tmem, approximately 9.5×10⁹ or more Tmem, approximately 9.6×10⁹ or more Tmem, approximately 9.7×10⁹ or more Tmem, approximately 9.8×10⁹ or more Tmem, approximately 9.9×10¹⁰ or more Tmem, approximately 1.0×10¹⁰ or more Tmem, approximately 1.1×10¹⁰ or more Tmem, approximately 1.2×10¹⁰ or more Tmem, approximately 1.3×10¹⁰ or more Tmem, approximately 1.4×10¹⁰ or more Tmem, approximately 1.5×10¹⁰ or more Tmem, approximately 1.6×10¹⁰ or more Tmem, approximately 1.7×10¹⁰ or more Tmem, approximately 1.8×10¹⁰ or more Tmem, approximately 1.9×10¹⁰ or more Tmem, approximately 2.0×10¹⁰ or more Tmem, approximately 2.1×10¹⁰ or more Tmem, approximately 2.2×10¹⁰ or more Tmem, approximately 2.3×10¹⁰ or more Tmem, approximately 2.4×10¹⁰ or more Tmem, approximately 2.5×10¹⁰ or more Tmem, approximately 2.6×10¹⁰ or more Tmem, approximately 2.7×10¹⁰ or more Tmem, approximately 2.8×10¹⁰ or more Tmem, approximately 2.9×10¹⁰ or more Tmem, approximately 3.0×10¹⁰ or more Tmem, approximately 3.1×10¹⁰ or more Tmem, approximately 3.2×10¹⁰ or more Tmem, approximately 3.3×10¹⁰ or more Tmem, approximately 3.4×10¹⁰ or more Tmem, approximately 3.5×10¹⁰ or more Tmem, approximately 3.6×10¹⁰ or more Tmem, approximately 3.7×10¹⁰ or more Tmem, approximately 3.8×10¹⁰ or more Tmem, approximately 3.9×10¹⁰ or more Tmem, approximately 4.0×10¹⁰ or more Tmem, approximately 4.1×10¹⁰ or more Tmem, approximately 4.2×10¹⁰ or more Tmem, approximately 4.3×10¹⁰ or more Tmem, approximately 4.4×10¹⁰ or more Tmem, approximately 4.5×10¹⁰ or more Tmem, approximately 4.6×10¹⁰ or more Tmem, approximately 4.7×10¹⁰ or more Tmem, approximately 4.8×110¹⁰ or more Tmem, approximately 4.9×10¹⁰ or more Tmem, or approximately 5.0×10¹⁰ or more Tmem (e.g., where Tmem are CD3+CD45RA−CD45RO+).

### Sequence and Timing of Administration of Cell Populations in an Allogeneic Hematopoietic Stem Cell Transplant (HSCT)

An aspect provides a multi-component pharmaceutical treatment or multi-component cellular therapy product to be administered to a human subject in need thereof. In some embodiments, the multi-component treatment comprises (a) a solution comprising a first population of CD45⁺ cells comprising hematopoietic stem and progenitor cells (HSPCs) and granulocytes wherein at most about 10% of the first population of CD45⁺ cells comprise granulocytes; (b) a solution comprising a population of cells enriched for regulatory T cells (Tregs), e.g., a second population of CD45⁺ cells; (c) a solution comprising a third population of CD45⁺ cells wherein the third population of CD45⁺ cells comprise at least about 20% CD3⁺ conventional T cells (Tcons), at least about 10% monocytes, and at least about 10% granulocytes; and (d) a solution comprising one or more doses of a graft vs host disease (GVHD) prophylactic agent. In some embodiments, the multi-component cellular therapy product comprises (a) a first single dose transfer bag comprising a first population of isolated CD45⁺ cells formulated with an excipient at a neutral pH, and comprising at least one dose of CD34⁺ hematopoietic stem and progenitor cells (HSPCs); (b) a second single dose transfer bag comprising a second population of isolated CD45⁺ cells formulated with an excipient at a neutral pH, and comprising at least one dose of fresh CD4⁺CD25⁺CD127^{dim} regulatory T cells (Tregs); and (c) a third single dose transfer bag comprising a third population of isolated CD45⁺ cells formulated with an excipient at a neutral pH, and comprising at least one dose of conventional CD3⁺ T cells (Tcons). In some embodiments, the multi-component cellular therapy product comprises (a) a population of CD34⁺ hematopoietic stem and progenitor cells (HSPCs); and (b) a population of conventional CD3⁺ T cells (Tcons), wherein the population of HSPCs, the population of Tcons, or the population of HSPCs and the population of Tcons comprises one or more cells comprising a chimeric receptor.

Another aspect provides a method of treating a human subject having or suspected of having a malignancy (e.g., a hematologic malignancy). In some embodiments, the human subject is or has been diagnosed with the hematologic malignancy. In some embodiments, the method comprises administering to the human subject a solution comprising the first population of CD45⁺ cells, a solution comprising the population of cells enriched for regulatory T cells (Tregs), e.g., a second population of CD45⁺ cells, a solution comprising the third population of CD45⁺ cells, and a solution comprising one or more doses of the GVHD prophylactic agent. In this embodiment, the solution comprising the first population of CD45⁺ cells, the solution comprising the population of cells enriched for regulatory Tregs, the solution comprising the third population of CD45⁺ cells, and the solution comprising one or more doses of the GVHD prophylactic agent are as defined according to any herein disclosed multi-component pharmaceutical treatment or multi-component cellular therapy product. In some embodiments, the method comprises administering to the human subject a solution comprising a first population of isolated CD45⁺ cells comprising CD34⁺ hematopoietic stem and progenitor cells (HSPCs), a solution comprising a second population of isolated CD45⁺ cells comprising fresh CD4⁺CD25⁺CD127^{dim} regulatory T cells (Tregs), and a solution comprising a third population of isolated CD45⁺ cells comprising conventional CD3⁺ T cells (Tcons). This embodiment may further comprise administering a solution comprising one or more doses of a graft vs host disease (GVHD) prophylactic agent. In this embodiment, the solution comprising the first population of CD45⁺ cells, the solution comprising the second population of CD45⁺ cells, the solution comprising the third population of CD45⁺ cells, and the solution comprising one or more doses of the GVHD prophylactic agent are as defined according to any herein disclosed multi-component pharmaceutical treatment or multi-component cellular therapy product. In some embodiments, the method comprises administering to the human subject a cellular therapy wherein the cellular therapy comprises: (a) one or more allogeneic cells comprising a chimeric receptor; and (b) an immune-modulating cell therapy product, wherein the one or more allogeneic cells and/or one or more cells of the immune-modulating cell therapy product are from an HLA-compatible donor, relative to the human subject.

Disclosed herein are methods for enhanced allogeneic hematopoietic stem cell transplantation, comprising administering to a subject solutions that comprise populations of cells.

In some embodiments, cell populations that comprise a first population of CD45⁺ cells which comprises at least HSPCs, a population of cells enriched for regulatory T cells (Tregs), and a third population of CD45⁺ cells which comprises at least Tcons are administered to a subject.

The first population of CD45⁺ cells and the population of cells enriched for Tregs can be administered at the same or similar times, or at different times. In some embodiments, first population of CD45⁺ cells and the population of cells enriched for Tregs are administered on the same day.

In various embodiments, the first population of CD45⁺ cells and the population of cells enriched for Tregs are administered before the third population of CD45⁺ cells.

The first population of CD45⁺ cells and the population of cells enriched for Tregs can administered at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours apart.

The third population of CD45⁺ cells can be administered to the subject after the first population of CD45⁺ cells.

The third population of CD45⁺ cells can be administered to the subject at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells.

In some embodiments, the third population of CD45⁺ cells is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or 120 hours after the first population of CD45⁺ cells.

The third population of CD45⁺ cells can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45⁺ cells.

The third population of CD45⁺ cells can be administered to the subject after the population of cells enriched for Tregs.

The population Tcons can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

In some embodiments, the third population of CD45⁺ cells is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

The third population of CD45⁺ cells can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of cells enriched for Tregs.

The third population of CD45⁺ cells can be administered to the subject after the first population of CD45⁺ cells and the population of cells enriched for Tregs.

The third population of CD45⁺ cells can be administered to the subject, for example, greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells and the population of cells enriched for Tregs.

In some embodiments, the third population of CD45⁺ cells is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells and the population of cells enriched for Tregs.

The third population of CD45⁺ cells can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45⁺ cells and the population of cells enriched for Tregs.

In some embodiments, a population of hematopoietic stem and progenitor cells (HSPCs), a population of cells enriched for regulatory T cells (Tregs), a population of conventional T cells (Tcons), and a population of invariant natural killer T cells (INKTs) are administered to a subject.

The population of iNKTs can be administered to the subject at the same time or at a similar time as the first population of CD45⁺ cells. In some embodiments, the population of iNKTs is administered to the subject after the first population of CD45⁺ cells.

The population of iNKTs can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells.

In some embodiments, the population of iNKTs is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells.

The population of iNKTs can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45⁺ cells.

The population of iNKTs can be administered to the subject at the same time or at a similar time as the population of cells enriched for Tregs. In some embodiments, the population of iNKTs is administered to the subject after the population of cells enriched for Tregs.

A population of iNKTs can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

In some embodiments, the population of iNKTs is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

The population of iNKTs can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of cells enriched for Tregs.

In some embodiments, a population of hematopoietic stem and progenitor cells (HSPCs), a population of cells enriched for regulatory T cells (Tregs), a population of conventional T cells (Tcons), and a population of memory T cells (Tmems) are administered to a subject.

A population of Tmems can be administered to the subject at the same time or at a similar time as the first population of CD45⁺ cells. In some embodiments, the population of Tmems is administered to the subject after the first population of CD45⁺ cells.

The population of Tmems can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells.

In some embodiments, the population of Tmems is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the first population of CD45⁺ cells.

The population of Tmems can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the first population of CD45⁺ cells.

The population of Tmems can be administered to the subject at the same time or at a similar time as the population of cells enriched for Tregs. In some embodiments, the population of Tmems is administered to the subject after the population of cells enriched for Tregs.

The population of Tmems can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

In some embodiments, the population of Tmems is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of cells enriched for Tregs.

The population of Tmems can be administered to the subject, for example, between approximately 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of cells enriched for Tregs.

In some embodiments, the population of Tcons is administered at least about 12 hours after the population of HSPCs, e.g., the population of Tcons is administered from approximately 24 to approximately 96 hours after the population of HSPCs or the population of Tcons is administered from approximately 36 to approximately 60 hours after the population of HSPCs.

In embodiments, the population of Tcons is administered at least about 12 hours after the population of cells comprising Tregs, e.g., the population of Tcons is administered from approximately 24 to approximately 96 hours after the population of cells comprising Tregs or the population of Tcons is administered from approximately 36 to approximately 60 hours after the population of cells comprising Tregs.

### Heterogenous Cell Populations

A further aspect provides a method of transplanting a conventional T cell (Tcons) population as a part of a treatment regimen for a hematologic malignancy in which the method reduces a risk and/or severity of an adverse event associated with the treatment regimen. The method comprises administering to the patient a population of regulatory T cells (Tregs) comprising Tregs and a liquid suspending the Tregs; administering to the patient a heterogenous cell population comprising lymphocytes, granulocytes, monocytes, and a liquid suspending the cells. In this aspect, at least about 30% of the lymphocytes comprise Tcons, and after administration of the cell populations, the patient has a reduced risk and/or severity of the adverse event as compared to hematologic malignancy patients who received Tcons but did not receive Tregs.

A yet further aspect provides a method of transplanting cell populations into a human patient as a part of a treatment regimen for a hematologic malignancy in which the method reduces a risk and/or severity of an adverse event associated with the treatment regimen. The method comprises providing a population of hematopoietic stem and progenitor cells (HSPCs) to be administered to the patient; the population of HSPCs comprising HSPCs and a liquid suspending the HSPCs; providing a population of regulatory T cells (Tregs) to be administered to the patient, the population of Tregs comprising Tregs and a liquid suspending the Tregs; and providing a heterogenous cell population to be administered to the patient, the heterogenous cell population comprising lymphocytes, granulocytes, monocytes and a liquid suspending the cells. In this aspect, at least about 30% of the lymphocyte comprise conventional T cells (Tcons) and after administration of the cell populations, the patient has a reduced risk and/or severity of the adverse event as compared to hematologic malignancy patients who received a Tcon cell population but did not receive a T-reg cell population.

In various embodiments, the heterogenous cell population comprises from approximately 0.2 to approximately 2.0 percent hematopoietic stem and progenitor cells.

In some embodiments, the hematologic malignancy is leukemia, acute leukemia, acute myeloid leukemia (AML), acute lymphoid leukemia (ALL), mixed phenotype acute leukemia (MPAL), chronic myelogenous leukemia (CML), multiple myeloma, lymphoma, Hodgkin's lymphoma, non-Hodgkin lymphoma, myelodysplastic syndrome, myeloproliferative syndrome, myelofibrosis, and blastic plasmacytoid dendritic cell neoplasm (BPDCN), or any combinations thereof.

In embodiments, a genetic expression level of the T-reg cells correlates to cells that were harvested from the donor within about 60 hours prior to administration to the patient.

In various embodiments, the number of T-reg cells in the T-reg population is about equal to the number of T-con cells in the heterogenous cell population. In some cases, the T-reg cells in the T-reg population inhibit activation of conventional T cells in the heterogenous cell population by the patient's healthy tissue by an amount up to approximately 20 percent.

In some embodiments, the peripheral blood of the patient exhibits an elevated ratio of Tregs to CD4⁺ T cells up to approximately 100 days after administration of the cell populations as compared to a healthy human subject that was not administered the cells populations.

In embodiments, at least about 50% of the cells in the HSPC's cell population are colony forming units.

In various embodiments, at least one of the cell populations has an elevated amount of granulocyte colony-stimulating factor as compared to non-mobilized blood. In some cases, the at least one cell populations is the heterogenous cell population.

An aspect provides a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending the cells, wherein at least about 30% of the lymphocyte comprise conventional T cells (Tcons); and administering to the patient over a period of time up to approximately 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

In some embodiments, a heterogenous cell population may be administered to a subject. A heterogenous cell population may comprise many different cell types found in the peripheral blood of a human donor. A heterogenous cell population may comprise granulocytes, monocytes, and lymphocytes. A heterogenous cell population may comprise T cells (such as Tcons, Tregs, Tmems, naïve T cells, CD4⁺ T cells, NK-T cells), B cells, NK cells, HSPCs, dendritic cells (such as plasmacytoid dendritic cells and myeloid dendritic cells) and other cell populations found in peripheral blood. A heterogenous cell population may be administered to a subject in addition to the other populations described herein. For instance, a heterogenous cell population may be administered with HSPCs as described herein. In some cases, a heterogenous cell population may be administered with HSPCs and Tregs as described herein. In some cases, a heterogenous cell population may be administered with Tregs as described herein. In some cases, a heterogenous cell population may be administered with Tcons as described herein. In some cases, a heterogenous cell population may be administered instead of the Tcon population as described herein.

In some embodiments, a heterogenous cell population administered to a subject may comprise a combination of granulocytes and monocytes. A combination of granulocytes and monocytes may comprise from 30% to 80% of the heterogenous cell population. At least 30% of the heterogenous cell population may comprise a combination of granulocytes and monocytes. At most 80% of the heterogenous cell population may comprise a combination of granulocytes and monocytes. In some cases, 30% to 40%, 30% to 50%, 30% to 60%, 30% to 70%, 30% to 80%, 40% to 50%, 40% to 60%, 40% to 70%, 40% to 80%, 50% to 60%, 50% to 70%, 50% to 80%, 60% to 70%, 60% to 80%, or 70% to 80% of the heterogenous cell population may comprise a combination of granulocytes and monocytes. In some cases, 30%, 40%, 50%, 60%, 70%, or 80% of the heterogenous cell population may comprise a combination of granulocytes and monocytes. In some cases, at least 30%, 40%, 50%, 60% or 70% of the heterogenous cell population may comprise a combination of granulocytes and monocytes. In some cases, at most 40%, 50%, 60%, 70%, or 80% of the heterogenous cell population may comprise a combination of granulocytes and monocytes.

In some embodiments, a heterogenous cell population administered to the subject may comprise lymphocytes. Lymphocytes comprise CD45⁺ cells. Lymphocytes may comprise from 8% to 50% of the heterogenous cell population. In some cases, at least 8% of the heterogenous cell population may comprise lymphocytes. In some cases, at most 50% of the heterogenous cell population may comprise lymphocytes. In some cases, 8% to 10%, 8% to 20%, 8% to 25%, 8% to 30%, 8% to 40%, 8% to 45%, 8% to 50%, 10% to 20%, 10% to 25%, 10% to 30%, 10% to 40%, 10% to 45%, 10% to 50%, 20% to 25%, 20% to 30%, 20% to 40%, 20% to 45%, 20% to 50%, 25% to 30%, 25% to 40%, 25% to 45%, 25% to 50%, 30% to 40%, 30% to 45%, 30% to 50%, 40% to 45%, 40% to 50%, or 45% to 50% of the heterogenous cell population may comprise lymphocytes. In some cases, 8%, 10%, 20%, 25%, 30%, 40%, 45%, or 50% of the heterogenous cell population may comprise lymphocytes. In some cases, at least 8%, 10%, 20%, 25%, 30%, 40% or 45% of the heterogenous cell population may comprise lymphocytes. In some cases, at most 10%, 20%, 25%, 30%, 40%, 45%, or 50% of the heterogenous cell population may comprise lymphocytes.

In some embodiments, lymphocytes in the heterogenous cell population may comprise Tcons. Tcons may comprise from 40% to 85% of the lymphocyte subset of the heterogenous cell population. In some cases, at least 40% of the lymphocyte subset may comprise Tcons. In some cases, at most 85% of the lymphocyte subset may comprise Tcons. In some cases, 40% to 50%, 40% to 60%, 40% to 65%, 40% to 70%, 40% to 75%, 40% to 80%, 40% to 85%, 50% to 60%, 50% to 65%, 50% to 70%, 50% to 75%, 50% to 80%, 50% to 85%, 60% to 65%, 60% to 70%, 60% to 75%, 60% to 80%, 60% to 85%, 65% to 70%, 65% to 75%, 65% to 80%, 65% to 85%, 70% to 75%, 70% to 80%, 70% to 85%, 75% to 80%, 75% to 85%, or 80% to 85% of the lymphocyte subset may comprise Tcons. In some cases, 40%, 50%, 60%, 65%, 70%, 75%, 80%, or 85% of the lymphocyte subset may comprise Tcons. In some cases, at least 40%, 50%, 60%, 65%, 70%, 75% or 80% of the lymphocyte subset may comprise Tcons. In some cases, at most 50%, 60%, 65%, 70%, 75%, 80%, or 85% of the lymphocyte subset may comprise Tcons.

In some embodiments, CD3⁺ lymphocytes in the heterogenous cell population may comprise CD4⁺ T cells. In some cases, 30% to 70% of the CD3⁺ lymphocyte subset may comprise CD4⁺ T cells. In some cases, at least 30% of the CD3⁺ lymphocyte subset may comprise CD4⁺ T cells. In some cases, at most 70% of the CD3⁺ lymphocyte subset may comprise CD4⁺ T cells. In some cases, 30% to 40%, 30% to 50%, 30% to 60%, 30% to 70%, 40% to 50%, 40% to 60%, 40% to 70%, 50% to 60%, 50% to 70%, or 60% to 70% of the CD3⁺ lymphocyte subset may comprise CD4⁺ T cells. In some cases, 30%, 40%, 50%, 60%, or 70% of the CD3⁺ lymphocyte subset may comprise CD4⁺ T cells. In some cases, at least 30%, 40%, 50% or 60% of the CD3⁺ lymphocyte subset may comprise CD4⁺ T cells. In some cases, at most 40%, 50%, 60%, or 70% of the CD3⁺ lymphocyte subset may comprise CD4⁺ T cells.

In some embodiments, CD3⁺ lymphocytes in the heterogenous cell population may comprise CD8⁺ T cells. In some cases, 20% to 65% of the CD3⁺ lymphocyte subset may comprise CD8⁺ T cells. In some cases, at least 20% of the CD3⁺ lymphocyte subset may comprise CD8⁺ T cells. In some cases, at most 65% of the CD3⁺ lymphocyte subset may comprise CD8⁺ T cells. In some cases, 20% to 30%, 20% to 40%, 20% to 50%, 20% to 60%, 20% to 65%, 30% to 40%, 30% to 50%, 30% to 60%, 30% to 65%, 40% to 50%, 40% to 60%, 40% to 65%, 50% to 60%, 50% to 65%, or 60% to 65% of the CD3⁺ lymphocyte subset may comprise CD8⁺ T cells. In some cases, 20%, 30%, 40%, 50%, 60%, or 65% of the CD3⁺ lymphocyte subset may comprise CD8⁺ T cells. In some cases, at least 20%, 30%, 40%, 50% or 60% of the CD3⁺ lymphocyte subset may comprise CD8⁺ T cells. In some cases, at most 30%, 40%, 50%, 60%, or 65% of the CD3⁺ lymphocyte subset may comprise CD8⁺ T cells.

In some embodiments, lymphocytes in the heterogenous cell population may comprise B cells. In some cases, 4% to 35% of the lymphocyte subset may comprise B cells. In some cases, at least 4% of the lymphocyte subset may comprise B cells. In some cases, at most 35% of the lymphocyte subset may comprise B cells. In some cases, 4% to 5%, 4% to 10%, 4% to 20%, 4% to 30%, 4% to 35%, 5% to 10%, 5% to 20%, 5% to 30%, 5% to 35%, 10% to 20%, 10% to 30%, 10% to 35%, 20% to 30%, 20% to 35%, or 30% to 35% of the lymphocyte subset may comprise B cells. In some cases, 4%, 5%, 10%, 20%, 30%, or 35% of the lymphocyte subset may comprise B cells. In some cases, at least 4%, 5%, 10%, 20% or 30% of the lymphocyte subset may comprise B cells. In some cases, at most 5%, 10%, 20%, 30%, or 35% of the lymphocyte subset may comprise B cells. B cells may be CD45⁺ CD19⁺ or CD45⁺CD19⁺CD3− cells.

In some embodiments, lymphocytes in the heterogenous cell population may comprise NK cells. In some cases, 4% to 35% of the lymphocyte subset may comprise NK cells. In some cases, at least 4% of the lymphocyte subset may comprise NK cells. In some cases, at most 35% of the lymphocyte subset may comprise NK cells. In some cases, 4% to 5%, 4% to 10%, 4% to 20%, 4% to 30%, 4% to 35%, 5% to 10%, 5% to 20%, 5% to 30%, 5% to 35%, 10% to 20%, 10% to 30%, 10% to 35%, 20% to 30%, 20% to 35%, or 30% to 35% of the lymphocyte subset may comprise NK cells. In some cases, 4%, 5%, 10%, 20%, 30%, or 35% of the lymphocyte subset may comprise NK cells. In some cases, at least 4%, 5%, 10%, 20% or 30% of the lymphocyte subset may comprise NK cells. In some cases, at most 5%, 10%, 20%, 30%, or 35% of the lymphocyte subset may comprise NK cells. NK cells may be CD45⁺ CD56⁺ or CD45⁺CD56⁺CD3− cells.

In some embodiments, CD3⁺ lymphocytes in the heterogenous cell population may comprise NK-T cells. In some cases, 3% to 30% of the CD3⁺ lymphocyte subset may comprise NK-T cells. In some cases, at least 4% of the CD3⁺ lymphocyte subset may comprise NK-T cells. In some cases, at most 35% of the CD3⁺ lymphocyte subset may comprise NK-T cells. In some cases, 3% to 5%, 3% to 10%, 3% to 20%, 3% to 30%, 5% to 10%, 5% to 20%, 5% to 30%, 10% to 20%, 10% to 30%, 20% to 30%, of the CD3⁺ lymphocyte subset may comprise NK-T cells. In some cases, 3%, 5%, 10%, 20% or 30% of the CD3⁺ lymphocyte subset may comprise NK-T cells. In some cases, at least 3%, 5%, 10% or 20% of the CD3⁺ lymphocyte subset may comprise NK-T cells. In some cases, at most 10%, 20% or 30% of the CD3⁺ lymphocyte subset may comprise NK-T cells. NK-T cells may be CD45⁺ CD56⁺ or CD45⁺CD56⁺CD3⁺ cells.

In some embodiments, lymphocytes in the heterogenous cell population may comprise CD34⁺ cells. In some cases, 0.1% to 2% of the lymphocyte subset may comprise CD34⁺ cells. In some cases, at least 0.1% of the lymphocyte subset may comprise CD34⁺ cells. In some cases, at most 2% of the lymphocyte subset may comprise CD34⁺ cells. In some cases, 0.1% to 0.5%, 0.1% to 1%, 0.1% to 1.5%, 0.1% to 2%, 0.5% to 1%, 0.5% to 1.5%, 0.5% to 2%, 1% to 1.5%, 1% to 2%, or 1.5% to 2% of the lymphocyte subset may comprise CD34⁺ cells. In some cases, 0.1%, 0.5%, 1%, 1.5%, or 2% of the lymphocyte subset may comprise CD34⁺ cells. In some cases, at least 0.1%, 0.5%, 1% or 1.5% of the lymphocyte subset may comprise CD34⁺ cells. In some cases, at most 0.5%, 1%, 1.5%, or 2% of the lymphocyte subset may comprise CD34⁺ cells.

### GVHD Prophylactic Agents

Subjects administered a composition of the disclosure (e.g., a cell component comprising a populations of cells described herein) and a GVHD prophylactic agent (for example, a single GVHD prophylactic agent) exhibit a low incidence of ≥grade 1 aGVHD, for example, a lower incidence of ≥grade 1 aGVHD than subjects that are administered an alternate composition. A single GVHD prophylactic agent can be a calcineurin inhibitor such as tacrolimus or another agent which acts on the same targets as tacrolimus or comprises an active fragment of tacrolimus. In some embodiments, a low dose of a GVHD prophylactic agent is tacrolimus with a target trough level (e.g., trough blood level) of approximately 5 ng/mL to approximately 10 ng/mL. In some embodiments, a low dose of a GVHD prophylactic agent is tacrolimus with a target trough level of approximately 4 ng/mL to approximately 6 ng/mL. In some embodiments, a low dose of a GVHD prophylactic agent is tacrolimus with a target trough level (e.g., target trough blood level) of approximately 1 ng/mL or more, approximately 2 ng/mL or more, approximately 3 ng/mL or more, approximately 4 ng/mL or more, approximately 5 ng/mL or more, approximately 6 ng/mL or more, approximately 7 ng/mL or more, approximately 8 ng/mL or more, approximately 9 ng/mL or more, approximately 10 ng/mL or more, approximately 11 ng/mL or more, approximately 12 ng/mL or more, approximately 13 ng/mL or more, approximately 14 ng/mL or more, approximately 15 ng/mL or more, approximately 16 ng/mL or more, approximately 17 ng/mL or more, approximately 18 ng/mL or more, approximately 19 ng/mL or more, or approximately 20 ng/mL or more.

In some embodiments, the single GVHD prophylactic agent is tacrolimus, and the tacrolimus is provided at a dose that ranges from approximately 0.01 mg per kilogram of body weight of the subject to approximately 0.50 mg per kilogram of body weight of the subject twice per day. In some embodiments, the tacrolimus is provided at a dose of approximately 0.01 mg per kilogram of body weight of the subject twice per day, approximately 0.02 mg per kilogram of body weight of the subject twice per day, approximately 0.03 mg per kilogram of body weight of the subject twice per day, approximately 0.04 mg per kilogram of body weight of the subject twice per day, approximately 0.05 mg per kilogram of body weight of the subject twice per day, approximately 0.06 mg per kilogram of body weight of the subject twice per day, approximately 0.07 mg per kilogram of body weight of the subject twice per day, approximately 0.08 mg per kilogram of body weight of the subject twice per day, approximately 0.09 mg per kilogram of body weight of the subject twice per day, approximately 0.10 mg per kilogram of body weight of the subject twice per day, approximately 0.11 mg per kilogram of body weight of the subject twice per day, approximately 0.12 mg per kilogram of body weight of the subject twice per day, approximately 0.13 mg per kilogram of body weight of the subject twice per day, approximately 0.14 mg per kilogram of body weight of the subject twice per day, approximately 0.15 mg per kilogram of body weight of the subject twice per day, approximately 0.16 mg per kilogram of body weight of the subject twice per day, approximately 0.17 mg per kilogram of body weight of the subject twice per day, approximately 0.18 mg per kilogram of body weight of the subject twice per day, approximately 0.19 mg per kilogram of body weight of the subject twice per day, approximately 0.20 mg per kilogram of body weight of the subject twice per day, approximately 0.21 mg per kilogram of body weight of the subject twice per day, approximately 0.22 mg per kilogram of body weight of the subject twice per day, approximately 0.23 mg per kilogram of body weight of the subject twice per day, approximately 0.24 mg per kilogram of body weight of the subject twice per day, approximately 0.25 mg per kilogram of body weight of the subject twice per day, approximately 0.26 mg per kilogram of body weight of the subject twice per day, approximately 0.27 mg per kilogram of body weight of the subject twice per day, approximately 0.28 mg per kilogram of body weight of the subject twice per day, approximately 0.29 mg per kilogram of body weight of the subject twice per day, approximately 0.30 mg per kilogram of body weight of the subject twice per day, approximately 0.31 mg per kilogram of body weight of the subject twice per day, approximately 0.32 mg per kilogram of body weight of the subject twice per day, approximately 0.33 mg per kilogram of body weight of the subject twice per day, approximately 0.34 mg per kilogram of body weight of the subject twice per day, approximately 0.35 mg per kilogram of body weight of the subject twice per day, approximately 0.36 mg per kilogram of body weight of the subject twice per day, approximately 0.37 mg per kilogram of body weight of the subject twice per day, approximately 0.38 mg per kilogram of body weight of the subject twice per day, approximately 0.39 mg per kilogram of body weight of the subject twice per day, approximately 0.40 mg per kilogram of body weight of the subject twice per day, approximately 0.41 mg per kilogram of body weight of the subject twice per day, approximately 0.42 mg per kilogram of body weight of the subject twice per day, approximately 0.43 mg per kilogram of body weight of the subject twice per day, approximately 0.44 mg per kilogram of body weight of the subject twice per day, approximately 0.45 mg per kilogram of body weight of the subject twice per day, approximately 0.46 mg per kilogram of body weight of the subject twice per day, approximately 0.47 mg per kilogram of body weight of the subject twice per day, approximately 0.48 mg per kilogram of body weight of the subject twice per day, approximately 0.49 mg per kilogram of body weight of the subject twice per day, or approximately 0.50 mg per kilogram of body weight of the subject twice per day.

A single GVHD prophylactic agent can be sirolimus. In some embodiments, a low dose of a GVHD prophylactic agent is sirolimus with a target trough level (e.g., trough blood level) of approximately 3 ng/mL to approximately 8 ng/mL. In some embodiments, a low dose of a GVHD prophylactic agent is sirolimus with a target trough level of approximately 4 ng/mL to approximately 8 ng/mL. In some embodiments, a low dose of a GVHD prophylactic agent is sirolimus with a target trough level (e.g., target trough blood level) of approximately 1 ng/mL or more, approximately 2 ng/mL or more, approximately 3 ng/mL or more, approximately 4 ng/mL or more, approximately 5 ng/mL or more, approximately 6 ng/mL or more, approximately 7 ng/mL or more, approximately 8 ng/mL or more, approximately 9 ng/mL or more, approximately 10 ng/mL or more, approximately 11 ng/mL or more, approximately 12 ng/mL or more, approximately 13 ng/mL or more, approximately 14 ng/mL or more, approximately 15 ng/mL or more, approximately 16 ng/mL or more, approximately 17 ng/mL or more, approximately 18 ng/mL or more, approximately 19 ng/mL or more, or approximately 20 ng/mL or more.

In some embodiments, the single GVHD prophylactic agent is sirolimus, and the sirolimus is provided at a dose that ranges from approximately 0.01 mg per kilogram of body weight of the subject to approximately 0.50 mg per kilogram of body weight of the subject twice per day. In some embodiments, the sirolimus is provided at a dose of approximately 0.01 mg per kilogram of body weight of the subject twice per day, approximately 0.02 mg per kilogram of body weight of the subject twice per day, approximately 0.03 mg per kilogram of body weight of the subject twice per day, approximately 0.04 mg per kilogram of body weight of the subject twice per day, approximately 0.05 mg per kilogram of body weight of the subject twice per day, approximately 0.06 mg per kilogram of body weight of the subject twice per day, approximately 0.07 mg per kilogram of body weight of the subject twice per day, approximately 0.08 mg per kilogram of body weight of the subject twice per day, approximately 0.09 mg per kilogram of body weight of the subject twice per day, approximately 0.10 mg per kilogram of body weight of the subject twice per day, approximately 0.11 mg per kilogram of body weight of the subject twice per day, approximately 0.12 mg per kilogram of body weight of the subject twice per day, approximately 0.13 mg per kilogram of body weight of the subject twice per day, approximately 0.14 mg per kilogram of body weight of the subject twice per day, approximately 0.15 mg per kilogram of body weight of the subject twice per day, approximately 0.16 mg per kilogram of body weight of the subject twice per day, approximately 0.17 mg per kilogram of body weight of the subject twice per day, approximately 0.18 mg per kilogram of body weight of the subject twice per day, approximately 0.19 mg per kilogram of body weight of the subject twice per day, approximately 0.20 mg per kilogram of body weight of the subject twice per day, approximately 0.21 mg per kilogram of body weight of the subject twice per day, approximately 0.22 mg per kilogram of body weight of the subject twice per day, approximately 0.23 mg per kilogram of body weight of the subject twice per day, approximately 0.24 mg per kilogram of body weight of the subject twice per day, approximately 0.25 mg per kilogram of body weight of the subject twice per day, approximately 0.26 mg per kilogram of body weight of the subject twice per day, approximately 0.27 mg per kilogram of body weight of the subject twice per day, approximately 0.28 mg per kilogram of body weight of the subject twice per day, approximately 0.29 mg per kilogram of body weight of the subject twice per day, approximately 0.30 mg per kilogram of body weight of the subject twice per day, approximately 0.31 mg per kilogram of body weight of the subject twice per day, approximately 0.32 mg per kilogram of body weight of the subject twice per day, approximately 0.33 mg per kilogram of body weight of the subject twice per day, approximately 0.34 mg per kilogram of body weight of the subject twice per day, approximately 0.35 mg per kilogram of body weight of the subject twice per day, approximately 0.36 mg per kilogram of body weight of the subject twice per day, approximately 0.37 mg per kilogram of body weight of the subject twice per day, approximately 0.38 mg per kilogram of body weight of the subject twice per day, approximately 0.39 mg per kilogram of body weight of the subject twice per day, approximately 0.40 mg per kilogram of body weight of the subject twice per day, approximately 0.41 mg per kilogram of body weight of the subject twice per day, approximately 0.42 mg per kilogram of body weight of the subject twice per day, approximately 0.43 mg per kilogram of body weight of the subject twice per day, approximately 0.44 mg per kilogram of body weight of the subject twice per day, approximately 0.45 mg per kilogram of body weight of the subject twice per day, approximately 0.46 mg per kilogram of body weight of the subject twice per day, approximately 0.47 mg per kilogram of body weight of the subject twice per day, approximately 0.48 mg per kilogram of body weight of the subject twice per day, approximately 0.49 mg per kilogram of body weight of the subject twice per day, or approximately 0.50 mg per kilogram of body weight of the subject twice per day.’

Methods for alloHSCT of the disclosure utilize tacrolimus. Combining tacrolimus with one or more cell populations in an alloHSCT regimen as disclosed herein is shown to result in surprising improvements in clinical outcomes.

An aspect provides a multi-component pharmaceutical treatment or multi-component cellular therapy product to be administered to a human subject in need thereof. In some embodiments, the multi-component treatment comprises (a) a solution comprising a first population of CD45⁺ cells comprising hematopoietic stem and progenitor cells (HSPCs) and granulocytes wherein at most about 10% of the first population of CD45⁺ cells comprise granulocytes; (b) a solution comprising a population of cells enriched for regulatory T cells (Tregs); (c) a solution comprising a third population of CD45⁺ cells wherein the third population of CD45⁺ cells comprise at least about 20% CD3⁺ conventional T cells (Tcons), at least about 10% monocytes, and at least about 10% granulocytes; and (d) a solution comprising one or more doses of a graft vs host disease (GVHD) prophylactic agent, wherein the GVHD prophylactic agent is tacrolimus. In some embodiments, the multi-component cellular therapy product comprises a) a first single dose transfer bag comprising a first population of isolated CD45⁺ cells comprising a dose of CD34⁺ hematopoietic stem and progenitor cells (HSPCs) formulated with an excipient at a neutral pH; b) a second single dose transfer bag comprising a second population of isolated CD45⁺ cells comprising a dose of fresh CD4⁺CD25⁺CD127^{dim} regulatory T cells (Tregs) formulated with an excipient at a neutral pH; and c) a third single dose transfer bag comprising a third population of isolated CD45⁺ cells comprising a dose of conventional CD3⁺ T cells (Tcons) formulated with an excipient at a neutral pH, wherein the excipient comprises one more cryoprotectants. In some embodiments, the multi-component cellular therapy product comprises (a) one or more allogeneic cells comprising a chimeric receptor; and (b) an immune-modulating cell therapy product, wherein the one or more allogeneic cells and/or one or more cells of the immune-modulating cell therapy product are from an HLA-compatible donor, relative to a human subject receiving the multi-component cellular therapy product.

In various embodiments, the tacrolimus is administered or dosed in an amount to maintain or that maintains a target blood level of at least about 3 ng/mL for at least about 20 days after administering the third population of CD45⁺ cells, in an amount to maintain a target blood level of approximately 4 ng/mL or more for at least about 40 days after administering the third population of CD45⁺ cells, and/or in an amount that maintains a target blood level of approximately 4 ng/mL or more for at least about 40 days after administering the third population of CD45⁺ cells. In some cases, the tacrolimus is administered in an amount that maintains a target blood level of at most about 10 ng/mL for at least 30 days after administering the third population of CD45⁺ cells.

In some embodiments, the tacrolimus is administered or dosed in amount to maintain or that maintains a target blood level of approximately 1 ng/mL to approximately 10 ng/mL in the human subject receiving the product for approximately 5 days or more, approximately 10 days or more, approximately 15 days or more, approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, approximately 90 days or more, approximately 95 days or more, approximately 100 days or more, approximately 105 days or more, approximately 110 days or more, approximately 115 days or more, approximately 120 days or more, approximately 125 days or more, approximately 130 days or more, approximately 135 days or more, approximately 140 days or more, approximately 145 days or more, or approximately 150 days or more after administration of the third population of CD45⁺ cells.

In some embodiments, the tacrolimus is administered for at least about 60 days after administering the third population of CD45⁺ cells, for at least about 90 days after administering the third population of CD45⁺ cells, for at most about 150 days after administering the third population of CD45⁺ cells, for at most about 120 days after administering the third population of CD45⁺ cells.

In some embodiments, the tacrolimus is formulated for oral administration or for intravenous administration.

In embodiments, a herein-disclosed method further comprises administering to the patient over a period of time up to approximately 180 days a single graft versus host disease (GVHD) prophylactic agent (GVHDPA) comprising tacrolimus (tacrolimus GHVDPA); wherein the tacrolimus GHVDPA is administered to maintain a concentration of tacrolimus in the patient's blood above a threshold level during the period of time; and wherein a risk and/or severity of GHVD is significantly reduced. In various embodiments, the threshold level is above about 4 ng of tacrolimus per ml of patient blood or the threshold level is above about 5 ng of tacrolimus per ml of patient blood. In some embodiments, the tacrolimus GHVDPA is administered to maintain a concentration of tacrolimus in the patients' blood below an upper threshold level during the period of time. In some cases, the upper threshold level is below about 10 ng of tacrolimus per ml of patient blood.

In embodiments, the tacrolimus graft versus host disease (GVHD) prophylactic agent (GVHDPA) is intravenously administered or orally administered. In various embodiments, administration of the tacrolimus graft versus host disease (GVHD) prophylactic agent (GVHDPA) is started from approximately 12 to approximately 24 hours after administration of the T-cons. In some cases, the tacrolimus GHVDPA is administered for a period of time up to approximately 90 days, is administered for a period of time up to approximately 60 days. In some embodiments, the tacrolimus GHVDPA is initially administered to the patient at approximately 0.03 mg/kg patient's actual or ideal body weight/day. In some cases, a dose of the tacrolimus GVHDPA administered to the patient is tapered starting at approximately 90 days after a first dose is administered to the patient or is tapered starting at approximately 45 days after a first dose is administered to the patient.

Tacrolimus is a macrolide that can exhibit immunosuppressive activity in vivo, and can prevent or reduce the activation of T-lymphocytes in response to antigenic or mitogenic stimulation. Tacrolimus can therefore be used to reduce the risk of GVHD in alloHSCT recipient subjects. However, methods disclosed herein—that utilize tacrolimus as a single-agent prophylactic—achieve superior clinical outcomes to those observed in other alloHSCT methods that utilize tacrolimus GVHD prophylactic. For example, in some embodiments alloHSCT methods of the disclosure that utilize tacrolimus achieve superior relapse-free survival compared to a standard of care regimen that comprises more potent GVHD prophylaxis with methotrexate plus tacrolimus, and even compared to an alloHSCT method that utilizes a drug with a similar target and mechanism of action (sirolimus). Thus, although tacrolimus can be referred to as a GVHD prophylactic herein, it can also contribute to additional therapeutic effects beyond or not directly related to GVHD prophylaxis. Therefore, as used herein, the term “tacrolimus as a single-agent GVHD prophylactic” also “includes tacrolimus as a single-agent prophylactic for additional therapeutics effects.” In other words, the term “tacrolimus as a single-agent prophylactic” and “tacrolimus as a single-agent GVHD prophylactic” are synonyms.

Treatment with tacrolimus as a single-agent prophylactic can result in, for example, decreased cytokine production and decreased T cell signal transduction. Tacrolimus can bind to the FKBP-12 protein and form a complex with calcium-dependent proteins, thereby inhibiting calcineurin phosphatase activity. This prevents or reduces the dephosphorylation and translocation of nuclear factor of activated T-cells (NFAT), a nuclear component thought to initiate gene transcription for the expression of lymphokines. Tacrolimus also inhibits the transcription for genes which encode IL-3, IL-4, IL-5, GM-CSF, and TNF, all of which are involved in the early stages of T-cell activation.

In some embodiments, tacrolimus as a single-agent prophylactic is administered to a subject orally. Absorption of tacrolimus from the gastrointestinal tract after oral administration can be incomplete and variable. The absolute bioavailability of tacrolimus in healthy subjects after oral administration can be 18±5%. The rate and extent of absorption can vary based on whether tacrolimus is given with food. In some embodiments, tacrolimus is administered parenterally, for example, intravenously or subcutaneously. In some embodiments, tacrolimus is administered by a topical, intramuscular, intradermal, intraperitoneal, intraspinal, or epidural route. Tacrolimus can be administered as an extended-release formulation. In some embodiments, tacrolimus is used as a free base. In some embodiments, tacrolimus is used as a pharmaceutically acceptable salt.

In some embodiments, methods of the disclosure can allow low doses of tacrolimus to be used. In some embodiments, a low dose of tacrolimus can improve donor T cell chimerism in a subject. In some embodiments, a low dose of tacrolimus can improve alloHSCT engraftment as disclosed herein. In some embodiments, a low dose of tacrolimus can reduce the incidence or relative risk of adverse effects that can be associated with high doses of tacrolimus, such as blurred vision, liver and kidney toxicity, seizures, tremors, hypertension, hypomagnesemia, diabetes mellitus, hyperkalemia, itching, insomnia, and confusion.

In some embodiments, the tacrolimus is initially administered to the human subject at approximately 0.03 mg/kg human subject's actual or ideal body weight/day or the tacrolimus is initially administered from approximately 12 hours to approximately 24 hours after the administering of the third population of CD45⁺ cells, as disclosed herein. In some embodiments, the tacrolimus is initially administered to the human subject at a dose that ranges from approximately 0.01 mg/kg human subject's actual or ideal body weight/day to approximately 0.50 mg/kg human subject's actual or ideal body weight/day or the tacrolimus is initially administered from approximately 12 hours to approximately 24 hours after the administering of the third population of CD45⁺ cells, as disclosed herein. In some embodiments, the tacrolimus is initially administered to the human subject at a dose that ranges from approximately 0.01 mg/kg human subject's actual or ideal body weight twice per day to approximately 0.50 mg/kg human subject's actual or ideal body weight twice per day, or the tacrolimus is initially administered from approximately 12 hours to approximately 24 hours after the administering of the third population of CD45⁺ cells, as disclosed herein.

A circulating level of tacrolimus can be monitored, and doses adjusted accordingly to achieve a target concentration. For example, a whole blood concentration of tacrolimus can be monitored, and doses adjusted and administered to achieve a target trough level. A target trough level of tacrolimus can be, for example, less than about 25 ng/mL, less than about 24 ng/mL, less than about 23 ng/mL, less than about 22 ng/mL, less than about 21 ng/mL, less than about 20 ng/mL, less than about 19 ng/mL, less than about 18 ng/mL, less than about 17 ng/mL, less than about 16 ng/mL, less than about 15 ng/mL, less than about 14 ng/mL, less than about 13 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a target trough level (e.g., trough blood level) of tacrolimus is about 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a target trough level (e.g., trough blood level) of tacrolimus is about 6 ng/mL to approximately 10 ng/mL. In some embodiments, a target trough level of tacrolimus is about 6 ng/mL to approximately 9 ng/mL. In some embodiments, a target trough level of tacrolimus is about 6 ng/mL to approximately 8 ng/mL. In some embodiments, a target trough level of tacrolimus is about 5 ng/mL to approximately 10 ng/mL. In some embodiments, a target trough level of tacrolimus is about 5 ng/mL to approximately 9 ng/mL. In some embodiments, a target trough level of tacrolimus is about 5 ng/mL to approximately 8 ng/mL. In some embodiments, a target trough level of tacrolimus is about 4 ng/mL to approximately 10 ng/mL. In some embodiments, a target trough level of tacrolimus is about 4 ng/mL to approximately 9 ng/mL. In some embodiments, a target trough level of tacrolimus is about 4 ng/mL to approximately 8 ng/mL.

In some embodiments, a dose of tacrolimus as a single-agent prophylactic or a target trough level (e.g., trough blood level) of tacrolimus can be adjusted based on a clinical parameter disclosed herein. For example, in some cases, a dose or a target trough level of tacrolimus can be reduced if a subject exhibits lower donor T cell chimerism than desired, e.g., a percent of peripheral blood donor-derived CD3⁺ cells that is less than 95%, less than 90%, less than 85%, less than 80%, less than 75%, less than 70%, less than 65%, less than 60%, less than 55%, less than 50%, or less than 45% when evaluated after a suitable amount of time after administration of a cell population of the disclosure, for example, at approximately 14 days, 15 days, 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 110 days, 120 days, 130 days, 140 days, 150 days, 160 days, 170 days, 180 days, or 1 year after administration of a cell population of the disclosure (e.g., a first population of CD45⁺ cells). In some embodiments, a target trough level of tacrolimus can be increased if a subject exhibits signs of GVHD as disclosed herein.

In some embodiments, a subject achieves at least about 80% chimerism at approximately day 30. In some embodiments, a subject achieves at least about 80% chimerism at approximately day 30 and has a target trough level of tacrolimus of between approximately 6.5 ng/mL and approximately 9 ng/mL.

Administration of tacrolimus to a subject can commence before or after administration of a cell population disclosed herein (e.g., a first population of CD45⁺ cells). In some embodiments, administration of tacrolimus to a subject commences before administration of a cell population disclosed herein (e.g., a first population of CD45⁺ cells). In some embodiments, administration of tacrolimus to a subject commences after administration of a cell population disclosed herein (e.g., a first population of CD45⁺ cells). In some embodiments, administration of tacrolimus to a subject commences at approximately the same time as administration of a cell population disclosed herein (e.g., a first population of CD45⁺ cells).

In some embodiments, administration of tacrolimus to a subject commences at least 1 day, at least 2 days, at least 3 days, at least 4 days, at least 5 days, at least 6 days, at least 7 days, at least 10 days, at least 14 days, or at least 20 days before administration of a cell population disclosed herein (e.g., a population of CD45⁺ cells). In some embodiments, administration of tacrolimus to a subject commences at most 1 day, at most 2 days, at most 3 days, at most 4 days, at most 5 days, at most 6 days, at most 7 days, at most 10 days, at most 14 days, or at most 20 days before administration of a cell population disclosed herein (e.g., a population of CD45⁺ cells). In some embodiments, administration of tacrolimus to a subject commences 1 day, 2 days, 3 days, 4 days, 5 days, 6 days, 7 days, 10 days, 14 days, or 20 days before administration of a cell population disclosed herein (e.g., a population of CD45⁺ cells). In some embodiments, administration of tacrolimus to a subject commences 1 day before administration of a first population of CD45⁺ cells. In some embodiments, administration of tacrolimus to a subject commences 1 day before administration of a third population of CD45⁺ cells.

In some embodiments, the tacrolimus is initially administered to the human subject at approximately 0.03 mg/kg human subject's actual or ideal body weight/day or the tacrolimus is initially administered from approximately 12 hours to approximately 24 hours after the administering of the third population of CD45⁺ cells, as disclosed herein. In some embodiments, the tacrolimus is initially administered to the human subject at a dose that ranges from approximately 0.01 mg/kg human subject's actual or ideal body weight twice per day to approximately 0.50 mg/kg human subject's actual or ideal body weight twice per day, or the tacrolimus is initially administered from approximately 12 hours to approximately 24 hours after the administering of the third population of CD45⁺ cells, as disclosed herein.

In some embodiments, administration of tacrolimus to a subject commences at least 1 day, at least 2 days, at least 3 days, at least 4 days, at least 5 days, at least 6 days, at least 7 days, at least 10 days, at least 14 days, or at least 20 days after administration of a cell population disclosed herein (e.g., a population of CD45⁺ cells). In some embodiments, administration of tacrolimus to a subject commences at most 1 day, at most 2 days, at most 3 days, at most 4 days, at most 5 days, at most 6 days, at most 7 days, at most 10 days, at most 14 days, or at most 20 days after administration of a cell population disclosed herein (e.g., a population of CD45⁺ cells). In some embodiments, administration of tacrolimus to a subject commences 1 day, 2 days, 3 days, 4 days, 5 days, 6 days, 7 days, 10 days, 14 days, or 20 days after administration of a cell population disclosed herein (e.g., a population of CD45⁺ cells). In some embodiments, administration of tacrolimus to a subject commences 1 day after administration of a population of CD45⁺ cells. In some embodiments, administration of tacrolimus to a subject commences 1 day after administration of a third population of CD45⁺ cells. In some embodiments, administration of tacrolimus to a subject commences the same day as a cell population of the disclosure is administered.

Tacrolimus can be administered to a subject for any amount of time after administration of a cell population of the disclosure (e.g., a population of CD45⁺ cells). In some embodiments, tacrolimus is administered to a subject for the first 7 days, 14 days, first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years after administration of a cell population of the disclosure (e.g., a population of CD45⁺ cells).

In some embodiments, tacrolimus is administered to a subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years after administration of a cell population of the disclosure (e.g., a population of CD45⁺ cells).

In various embodiments, a dose of the tacrolimus is tapered starting at approximately 90 days after the first dose is administered to the human subject or a dose of the tacrolimus is tapered starting at approximately 45 days after the first dose is administered to the human subject.

In some embodiments, a subject achieves at least about 80% chimerism at approximately day 30. In some embodiments, a subject achieves at least about 80% chimerism at approximately day 30 and has a target trough level is of approximately 6.5 ng/mL and approximately 9 ng/mL.

### Subjects

Provided herein are compositions for administration to a recipient subject having a cancer, and methods of administering the same. The compositions and methods can be useful for treating or reducing cancer in the subject. In some embodiments, a third population of CD45⁺ cells that comprises, at least, Tcons is administered to the subject in order to elicit graft-versus-tumor (GVT) immune responses and with reduced graft versus host disease (GVHD).

In some embodiments, a subject is at least 3, at least 4, at least 5, at least 6, at least 7 at least 8 at least 9 at least 10, at least 11, or at least 12 months of age. In some embodiments, a subject is at least 1, least 2, least 3, least 4, least 5, least 6, at least 7, at least 8, at least 9, at least 10, at least 11, at least 12, least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, or at least 80 years of age. In some embodiments, a subject is approximately 18 years of age or older. In some embodiments, a subject is approximately 16 years of age or older. In some embodiments, a subject is approximately 13 years of age or older. In some embodiments, a subject is approximately 6 years of age or older. In some embodiments, a subject is approximately 3 years of age or older. In some embodiments, a subject is approximately 1 year of age or older. In some embodiments, a subject is approximately 9 months of age or older. In some embodiments, a subject is approximately 6 months of age or older. In some embodiments, a subject is approximately 3 months of age or older.

In some embodiments, a subject is at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, or at most 80 years of age. In some embodiments, a subject is at most 65 years of age. In some embodiments, a subject is at most 70 years of age. In some embodiments, a subject is at most 75 years of age.

In some embodiments, a subject is from between approximately 3 months to approximately 18 years of age. In some embodiments, a subject is from approximately 18 years to approximately 65 years of age. In some embodiments, a subject is from approximately 18 years to approximately 75 years of age. In some embodiments, a subject is from approximately 66 years to approximately 75 years of age.

In some embodiments, the subject has received from one to five previous lines of therapy prior to administration of a composition, treatment, and/or therapy of the present disclosure. In some embodiments, the subject has received five previous lines of therapy prior to administration of a composition, treatment, and/or therapy of the present disclosure. In some embodiments, the subject has received four previous lines of therapy prior to administration of a composition, treatment, and/or therapy of the present disclosure. In some embodiments, the subject has received three previous lines of therapy prior to administration of a composition, treatment, and/or therapy of the present disclosure. In some embodiments, the subject has received two previous lines of therapy prior to administration of a composition, treatment, and/or therapy of the present disclosure. In some embodiments, the subject has received one previous line of therapy prior to administration of a composition, treatment, and/or therapy of the present disclosure. In some embodiments, the subject is or has become refractory to one or more of the previous lines of therapy prior to administration of a composition, treatment, and/or therapy of the present disclosure. In some embodiments, the subject is or has become refractory to two or more of the previous lines of therapy prior to administration of a composition, treatment, and/or therapy of the present disclosure. In some embodiments, the subject is or has become refractory to three or more of the previous lines of therapy prior to administration of a composition, treatment, and/or therapy of the present disclosure. In some embodiments, the subject is or has become refractory to four or more of the previous lines of therapy prior to administration of a composition, treatment, and/or therapy of the present disclosure. In some embodiments, the subject is or has become refractory to all previous lines of therapy prior to administration of a composition, treatment, and/or therapy of the present disclosure.

### Conditions in an Allogeneic Hematopoietic Stem Cell Transplant

Another aspect provides a method of treating a human subject having (e.g., diagnosed with) or suspected of having a malignancy (e.g., a hematologic malignancy). The method comprises administering to the human subject a solution comprising the first population of CD45⁺ cells, a solution comprising the population of cells enriched for regulatory T cells (Tregs), a solution comprising the third population of CD45⁺ cells, and a solution comprising one or more doses of the GVHD prophylactic agent (e.g., tacrolimus). In this aspect, the solution comprising the first population of CD45⁺ cells, the solution comprising the population of cells enriched for regulatory Tregs, the solution comprising the third population of CD45⁺ cells, and the solution comprising one or more doses of the GVHD prophylactic agent are as defined according to any herein disclosed multi-component pharmaceutical treatment. Alternatively, the method comprises administering to the human subject a multi-component cellular therapy product of the present disclosure, a multi-component pharmaceutical treatment of the present disclosure, or a multi-component cellular therapy multi-component cellular therapy comprising: (a) a solution comprising a first population of isolated CD45⁺ cells comprising CD34⁺ hematopoietic stem and progenitor cells (HSPCs); (b) a solution comprising a second population of isolated CD45⁺ cells comprising fresh CD4⁺CD25⁺CD127^{dim} regulatory T cells (Tregs); and (c) a solution comprising a third population of isolated CD45⁺ cells comprising conventional CD3⁺ T cells (Tcons). In some embodiments, the method comprises administering to the human subject a multi-component cellular therapy product, wherein the multi-component cellular therapy product comprises (a) one or more allogeneic cells comprising a chimeric receptor; and (b) an immune-modulating cell therapy product, wherein the one or more allogeneic cells and/or one or more cells of the immune-modulating cell therapy product are from an HLA-compatible donor, relative to a human subject receiving the multi-component cellular therapy product.

In some embodiments, a hematologic malignancy that may be treating by a method of the present disclosure includes, without limitation, acute leukemia in complete remission (CR), active leukemia, primary refractory acute leukemia or acute leukemia with minimal residual disease, low-risk acute myeloid leukemia (AML), high-risk acute myeloid leukemia (AML) in complete remission, chronic myelogenous leukemia (CML), high-risk myelodysplastic syndrome, therapy-related myelodysplastic syndrome and/or secondary myelodysplastic syndrome, myeloproliferative syndrome, non-Hodgkin lymphoma with poor risk features not suitable for autologous hematopoietic cell transplant (HCT), acute lymphoblastic leukemia (ALL), multiple myeloma, and any combinations thereof. In some embodiments, the human subject has been diagnosed with a histopathologically confirmed hematologic malignancy of the present disclosure. In some embodiments, the hematologic malignancy risk category (e.g., low risk, intermediate risk, high risk, very high risk) may be determined as per the Center for International Blood & Marrow Transplant Research (CIBMTR) Disease Risk Index (DRI).

In some embodiments, a myelodysplastic syndrome (MDS) of the present disclosure is one that is indicated for allogeneic hematopoietic cell transplant (alloHCT) per the 2017 International Expert Panel recommendations (de Witte 2017) and/or is a therapy-related/secondary MDS as defined by the World Health Organization classification of myeloid malignancies (WHO 2017).

In some embodiments, the acute leukemia is in complete remission with incomplete hematologic recovery (CRi). In any of the preceding embodiments, minimal residual disease may or may not be present in the human subject. In some embodiments, the acute leukemia is categorized as intermediate-risk to high-risk acute leukemia or very high-risk leukemia. The acute leukemia may be acute myeloid leukemia (AML), acute lymphoid leukemia (ALL), and/or mixed phenotype acute leukemia (MPAL). In some embodiments, the ALL is B cell ALL. In some embodiments, the human subject is in complete response with a history of chemotherapy refractory disease. In some embodiments, the chemotherapy refractory disease comprises progression or stable disease after one line of chemotherapy. In some embodiments, the human subject is in complete response with CRLF2 rearrangement, Ph-like phenotype, MLL/KMT2a rearrangement, and/or hypodiploid karyotype. In some embodiments, the Ph-like phenotype comprises Ph+ ALL. In some embodiments, wherein the human subject has persistent or relapsed minimal residual disease (MRD). In some embodiments, the human subject has active B-cell ALL. In some embodiments, the human subject has approximately 5% or greater bone marrow blasts, circulating blasts, and/or extramedullary disease. In some embodiments, the B-cell ALL is CD19⁺.

In some embodiments, the human subject may have active disease at time of treatment. In some embodiments, the human subject may have approximately 20% or less, approximately 19% or less, approximately 18% or less, approximately 17% or less, approximately 16% or less, approximately 15% or less, approximately 14% or less, approximately 13% or less, approximately 12% or less, approximately 11% or less, approximately 10% or less, approximately 9% or less, approximately 8% or less, approximately 7% or less, approximately 6% or less, or approximately 5% or less blast burden in their bone marrow.

In some embodiments, for acute myeloid leukemia and mixed phenotype acute leukemia, a complete response/complete remission (CR) may be according to FDA draft guidance for industry (“Acute Myeloid Leukemia: Developing Drugs and Biological Products for Treatment (August 2020)”. In some embodiments, a CRi may comprise meeting all CR criteria except for residual neutropenia or thrombocytopenia.

In some embodiments, for acute lymphoblastic leukemia (ALL), a CR is defined per the Center for International Blood and Marrow Transplant Research (CIBMTR 2021). In some embodiments, a CRi may comprise meeting all CR criteria except for residual neutropenia or thrombocytopenia.

A further aspect provides a method of transplanting a conventional T cell (Tcons) population as a part of a treatment regimen for a hematologic malignancy in which the method reduces a risk and/or severity of an adverse event associated with the treatment regimen. The method comprises administering to the patient a population of regulatory T cells (Tregs) comprising Tregs and a liquid suspending the Tregs; administering to the patient a heterogenous cell population comprising lymphocytes, granulocytes, monocytes, and a liquid suspending the cells. In this aspect, at least about 30% of the lymphocytes comprise Tcons, and after administration of the cell populations, the patient has a reduced risk and/or severity of the adverse event as compared to hematologic malignancy patients who received Tcons but did not receive Tregs.

A yet further aspect provides a method of transplanting cell populations into a human patient as a part of a treatment regimen for a hematologic malignancy in which the method reduces a risk and/or severity of an adverse event associated with the treatment regimen. The method comprises providing a population of hematopoietic stem and progenitor cells (HSPCs) to be administered to the patient; the population of HSPCs comprising HSPCs and a liquid suspending the HSPCs; providing a population of regulatory T cells (Tregs) to be administered to the patient, the population of Tregs comprising Tregs and a liquid suspending the Tregs; and providing a heterogenous cell population to be administered to the patient, the heterogenous cell population comprising lymphocytes, granulocytes, monocytes and a liquid suspending the cells. In this aspect, at least about 30% of the lymphocytes comprise conventional T cells (Tcons) and after administration of the cell populations, the patient has a reduced risk and/or severity of the adverse event as compared to hematologic malignancy patients who received a Tcon cell population but did not receive a T-reg cell population.

Another aspect provides a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending the cells, wherein at least about 30% of the lymphocyte comprise conventional T cells (Tcons); and administering to the patient over a period of time up to approximately 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

The methods of the disclosure can be used for treating a subject (e.g., a human subject) with a cancer. In some embodiments, the subject has been treated for cancer, e.g., by treatment with a chemotherapeutic drug or with radiation. The methods of the disclosure can be useful for treating a hematologic malignancy, for example, leukemia or lymphoma. Examples of hematologic malignancies that can be treated by the methods of the disclosure include, but are not limited to, acute lymphocytic leukemia (ALL), acute myelogenous leukemia (AML), chronic myelogenous leukemia (CML), multiple myeloma, lymphomas such as Hodgkin and non-Hodgkin lymphomas, myelodysplastic syndrome, myelofibrosis, and blastic plasmacytoid dendritic cell neoplasm (BPDCN). A cancer can be a solid tumor. In some embodiments, the cancer is a primary or metastatic tumor.

The types of cancer that can be treated using the methods of the present disclosure include but are not limited to leukemia, lymphoma, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and pharyngeal cancer, acute lymphocytic leukemia, acute myeloid leukemia, children's leukemia, chronic lymphocytic leukemia, chronic myeloid leukemia, liver cancer, lung cancer, lung carcinoid tumors, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, nonmelanoma skin cancers, stomach cancer, testicular cancer; thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarcinoma, head and neck cancers, teratocarcinoma, and Waldenstrom's macroglobulinemia.

Patients with high-risk hematologic malignancies are rarely cured with standard chemotherapy. High-risk malignancies include, for example, leukemia or lymphoma that has progressed beyond first remission, or leukemia or lymphoma with refractory relapse.

In some embodiments, the human subject or patient has previously been or is concurrently treated for the hematologic malignancy.

A subject that receives a composition of the disclosure can have, for example, acute myeloid leukemia, acute lymphoid leukemia, mixed phenotype leukemia, myelofibrosis, high-risk myelodysplastic syndrome, very high-risk myelodysplastic syndrome, myelofibrosis (MF) that is eligible for transplant per National Comprehensive Cancer Network Guidelines, intermediate-2- or high-risk MF according to the IPSS, DIPSS or DIPSS-plus scoring systems, intermediate-1-risk MF associated with high-risk features such as high symptoms burden, low platelet counts, or complex cytogenetics, primary myelofibrosis, myelofibrosis evolved from another myeloproliferative neoplasm, myelodysplastic syndrome, non-Hodgkin lymphoma, a non-malignant indication for allogeneic hematopoietic stem cell transplantation (alloHSCT),

In some embodiments, a subject has acute myeloid leukemia. In some embodiments, a subject has acute lymphoid leukemia. In some embodiments, a subject has mixed phenotype leukemia. In some embodiments, a subject has high-risk myelodysplastic syndrome. In some embodiments, a subject has very high-risk myelodysplastic syndrome. In some embodiments, a subject has myelofibrosis (MF) that is eligible for transplant per National Comprehensive Cancer Network Guidelines. In some embodiments, a subject has intermediate-2- or high-risk myelofibrosis according to the IPSS, DIPSS or DIPSS-plus scoring systems. In some embodiments, a subject has intermediate-1-risk myelofibrosis associated with high-risk features such as high symptoms burden, low platelet counts, or complex cytogenetics. In some embodiments, a subject has primary myelofibrosis. In some embodiments, a subject has myelofibrosis. In some embodiments, a subject has myelofibrosis evolved from another myeloproliferative neoplasm. In some embodiments, a subject has myelodysplastic syndrome. In some embodiments, a subject has non-Hodgkin lymphoma. In some embodiments, a subject has a non-malignant indication for alloHSCT. In some embodiments, a subject has blastic plasmacytoid dendritic cell neoplasm (BPDCN).

A subject can be in complete remission (CR). A subject can be in complete remission with incomplete hematologic recovery (CRi), e.g., without the presence of known minimal residual disease. A subject can have minimal residual disease. A subject can have no evidence of minimal residual disease. A subject can have active disease. A subject can have a leukemia (e.g., acute myeloid, acute lymphoid, or mixed phenotype) that is not in morphologic CR with bone marrow infiltration by leukemic blasts of ≤10%. A subject can have a leukemia (e.g., acute myeloid, acute lymphoid, or mixed phenotype) that is in morphologic CR with evidence of minimal residual positivity by either multiparameter flow cytometric analysis or by a nucleic acid-based technique.

Complete remission (CR) for acute myeloid, lymphoid or mixed phenotype leukemia can be indicated by meeting all of the following criteria: (i) Bone marrow blasts <5%; (ii) Absence of circulating blasts and blasts with Auer rods; (ii) Absence of extramedullary disease 4. ANC≥1.0×10⁹/L (1,000/μL); (iii) Platelet count≥100×10⁹/L (100,000/μL); and (iv) Independence of red cell transfusions. Complete Response with Incomplete Hematologic Recovery (CRi) can be indicated by meeting all the CR criteria except for residual neutropenia (<1.0×10⁹/L) or thrombocytopenia (<100×10⁹/L).

Another aspect provides methods for treating a human subject having (e.g., diagnosed) or suspected of having multiple sclerosis with any of the compositions, treatments, or cellular therapy products of the present disclosure. In some embodiments, the multiple sclerosis is primary progressive multiple sclerosis, chronic progressive multiple sclerosis, secondary progressive multiple sclerosis, relapsing-remitting multiple sclerosis, acute relapsing multiple sclerosis, or any combination thereof.

In some embodiments, the methods of the present disclosure may further include administering one or more additional therapeutic agents. Examples, of such therapeutic agents include, without limitation, an inhibitor of an immunosuppressive enzymes, e.g., narginase II and indoleamine 2,3-dioxygenase 1 (IDO1); a proinflammatory cytokine, e.g., IL-2 or IL-15; a blocker of an anti-inflammatory cytokine, e.g., an antibody that binds to and/or blocks M-CSF, IL-4, or TGF-beta; a bispecific antibody, e.g., that targets a protein or proteins relating to Natural Killer (NK) function, such as an NKG2D-based or CD16-based bispecific antibody; a check-point inhibitor, e.g., a CTLA4 inhibitor, a PD1 inhibitor, a PD-L1 inhibitor, a CD47/SIRP-alpha inhibitor, a LAG-3 inhibitor, a TIM-3 inhibitor, a B7-H3 inhibitor, a B7-H4 inhibitor, an A2aR inhibitor, a CD73 inhibitor, an NKG2A inhibitor, a PVRIG/PVRL2 inhibitor, a CEACAM1 inhibitor, a CEACAM 5/6 inhibitor, a FAK inhibitor, a CCL2/CCR2 inhibitor, a LIF inhibitor, a CSF-1 inhibitor, an IL-8 inhibitor, a SEMA4D inhibitor, an Ang-2 inhibitor, a CLEVER-1 inhibitor, an Axl inhibitor, a phosphatidylserine inhibitor, and/or a TIGIT inhibitor; a JAK/STAT inhibitor, e.g., a Jak2 inhibitor; a FLT3 inhibitor.

### Sensitivities

In some embodiments, a subject does not have a known allergy or hypersensitivity to, or intolerance of, tacrolimus. In some embodiments, a subject does not have a known allergy or hypersensitivity to, or intolerance of, sirolimus.

In some embodiments, subjects are not sensitive to iron dextran (e.g., subjects with sensitivity to iron dextran are not eligible to receive a composition of the disclosure. In some cases, this may be because of the magnetic beads used in some embodiments to isolate, deplete, and/or purify cell types).

In some embodiments, subjects are not sensitive to products derived from cyanine dyes (e.g., subjects with sensitivity to products derived from cyanine dyes are not eligible to receive a composition of the disclosure).

In some embodiments, subjects are not sensitive to proteins products derived from murine sources (e.g., subjects with sensitivity to proteins products derived from murine sources are not eligible to receive a composition of the disclosure).

In some embodiments, subjects are not sensitive to proteins products derived from bovine sources (e.g., subjects with sensitivity to proteins products derived from bovine sources are not eligible to receive a composition of the disclosure).

In some embodiments, subjects are not sensitive to proteins products derived from algal sources (e.g., subjects with sensitivity to proteins products derived from algal sources are not eligible to receive a composition of the disclosure).

In some embodiments, subjects are not sensitive to proteins products derived from Streptomyces avidinii (e.g., subjects with sensitivity to proteins products derived from Streptomyces avidinii are not eligible to receive a composition of the disclosure).

### Organ Function and Biomarkers

A subject can have an estimated glomerular filtration rate (eGFR) >30 mL/minute. A subject can have an estimated glomerular filtration rate (eGFR) >40 mL/minute. A subject can have an eGCF of >50 mL/minute. A subject can have an estimated glomerular filtration rate (eGFR) >60 mL/minute.

A subject can have a cardiac ejection fraction at rest ≥45%, or shortening fraction of ≥27% by echocardiogram or radionuclide scan (MUGA).

A subject can have a diffusing capacity of the lung for carbon monoxide (DLCO) (adjusted for hemoglobin) of ≥50%.

A subject can have a negative serum or urine beta-HCG test, e.g., in females of childbearing potential within 3 weeks of registration.

A subject can have total bilirubin <2 times upper limit of normal (ULN).

A subject can have Gilbert's syndrome, wherein hemolysis has been excluded.

A subject can have an ALT reading within 3 times upper limit of normal (ULN). A subject can have an AST reading within 3 times upper limit of normal (ULN).

### Additional Therapies and Other Subject Characteristics

In addition to a subject receiving both allogeneic cells that include a chimeric receptor and an immune-modulating cell therapy product (e.g., alloHSCTs), subjects may receive additional therapies. In some embodiments, a subject has not received a prior alloHSCT. In some embodiments, a subject is not a candidate for autologous transplant. In some embodiments, a subject is not receiving corticosteroids or other immunosuppressive therapy. In some embodiments, a subject is receiving topical corticosteroids or oral systemic corticosteroid doses less than or equal to 10 mg/day. In some embodiments, a subject does not receive donor lymphocyte infusion (DLI). In some embodiments, a subject does not receive a T cell depleting pharmaceutical, e.g., post-transplant cyclophosphamide (Cy), peri-transplant anti-thymocyte globulin (ATG), or alemtuzumab. In some embodiments, a subject that has previously been exposed to a T cell-depleting agent has a 5-half-life washout of the agent prior to planned transplant day 0 (day of infusion of the Treg and HSPC components of the graft). In some embodiments, a subject is not positive for anti-donor HLA antibodies against a mismatched allele in the selected donor as determined by either: (a) A positive crossmatch test of any titer; or (b) The presence of anti-donor HLA antibody to any HLA locus. In some embodiments, the subject has a Karnofsky performance score ≥70%. In some embodiments, a subject does not have a hematopoietic cell transplantation-specific Comorbidity Index (HCT-CI) of >4. In some embodiments, a subject does not have an uncontrolled bacterial, viral, or fungal infection. In some embodiments, a subject is not taking antimicrobial therapy and with progression or no clinical improvement in infection. In some embodiments, a subject is not seropositive for HIV-1 or -2, HTLV-1 or -2, Hepatitis B sAg, or Hepatitis C antibody. In some embodiments, a subject does not have an uncontrolled autoimmune disease that requires active immunosuppressive treatment. In some embodiments, a subject does has not had concurrent malignancies or active disease within 1 year, for example, excluding non-melanoma skin cancers that have been curatively resected. In some embodiments, a subject does not exhibit psychosocial circumstances that preclude the patient being able to go through transplant or participate responsibly in follow up care. In some embodiments, a subject is not pregnant or breastfeeding. In some embodiments, a subject does not have a serious medical condition or abnormality in clinical laboratory tests that, in the medical professional's judgment, precludes the subject's safety upon receipt of a composition of the disclosure. In some embodiments, a subject is eligible for myeloablative alloHSCT.

In some embodiments, a subject receives a prophylactic agent to reduce the risk of bacterial, fungal, and/or viral infection, e.g., during the peri-transplant period.

In some embodiments, a subject receives a supportive therapy for HCT-related toxicity. In some embodiments, a subject does not receive a supportive therapy for HCT-related toxicity. In some embodiments, a subject receives a growth factor. In some embodiments, a subject does not receive a growth factor. In some embodiments, a subject receives intravenous immunoglobulin. In some embodiments, a subject does not receive intravenous immunoglobulin. In some embodiments, a subject receives an analgesic. In some embodiments, a subject does not receive an analgesic. In some embodiments, a subject receives an anti-emetic. In some embodiments, a subject does not receive an anti-emetic. In some embodiments, a subject receives electrolyte replacement. In some embodiments, a subject does not receive electrolyte replacement. In some embodiments, a subject receives a tyrosine kinase inhibitor (e.g., a FLT3 inhibitor). In some embodiments, a subject does not receive a tyrosine kinase inhibitor (e.g., a FLT3 inhibitor). In some embodiments, a subject receives prednisone or an equivalent thereof, e.g., at a dose of ≤10 mg/day. In some embodiments, a subject does not receive prednisone or an equivalent thereof. In some embodiments, a subject receives corticosteroid treatment to manage GVHD. In some embodiments, a subject does not receive corticosteroid treatment to manage GVHD. In some embodiments, a subject receives high-dose corticosteroid treatment to manage GVHD. In some embodiments, a subject does not receive high-dose corticosteroid treatment to manage GVHD. In some embodiments, a subject receives corticosteroid treatment to manage, for example, adrenal insufficiency, hypersensitivity reactions, or other non-cancer-related symptoms including premedication for known hypersensitivity reactions to contrast for scans. In some embodiments, a subject does not receive corticosteroid treatment. In some embodiments, a subject receives an immunosuppressive medication. In some embodiments, a subject does not receive an immunosuppressive medication. In some embodiments, a subject receives a donor lymphocyte infusion. In some embodiments, a subject does not receive a donor lymphocyte infusion.

### Conditioning Regimens

Conditioning regimens can be used as part of an alloHSCT regimen and/or regimens including administration of allogeneic cells comprising a chimeric receptor (e.g., a TCR or a CAR-T) of the disclosure. Chemotherapy and/or irradiation given soon before a transplant is called a conditioning regimen. Conditioning regimens can help eradicate a patient's disease prior to the infusion of HSPCs, suppress immune reactions, and allow a donor HSPCs to reconstitute the vacant hematopoietic compartment that results from the conditioning regimen. In some embodiments of the methods of the disclosure, a subject can be treated with myeloablative conditioning prior to infusion of cell populations described herein. In some embodiments of the methods of the disclosure, a subject can be treated with myeloreductive conditioning prior to infusion of cell populations described herein. In some embodiments of the methods of the disclosure, a subject can be treated with a reduced intensity myeloablative conditioning prior to infusion of cell populations described herein. In some embodiments of the methods of the disclosure, a subject can be treated with non-myeloablative conditioning prior to administering a cell population or cell populations described herein.

As used herein, the term conditioning regimen and the like applies to myeloablative conditioning, myeloreductive conditioning, reduced intensity myeloablative therapy/conditioning, and/or non-myeloablative conditioning. As used herein, the term myeloablative therapy/conditioning also includes myeloreductive conditioning and reduced intensity myeloablative conditioning.

In aspects and embodiments, a treatment and/or method further comprises a conditioning regimen, wherein the conditioning regimen is administered before administration of one of (a) a solution comprising a first population of CD45⁺ cells comprising hematopoietic stem and progenitor cells (HSPCs) and granulocytes wherein at most about 10% of the first population of CD45⁺ cells comprise granulocytes; (b) a solution comprising a population of cells enriched for regulatory T cells (Tregs); and (c) a solution comprising a third population of CD45⁺ cells wherein the third population of CD45⁺ cells comprise at least about 20% CD3⁺ conventional T cells (Tcons), at least about 10% monocytes, and at least about 10% granulocytes; and (d) a solution comprising one or more doses of a graft vs host disease (GVHD) prophylactic agent. In aspects and embodiments, a treatment and/or method further comprises a conditioning regimen, wherein the conditioning regimen is administered before administration of (a) a solution comprising a first population of isolated CD45⁺ cells comprising CD34⁺ hematopoietic stem and progenitor cells (HSPCs); (b) a solution comprising a second population of isolated CD45⁺ cells comprising fresh CD4⁺ CD25⁺ CD127^{dim} regulatory T cells (Tregs); and (c) a solution comprising a third population of isolated CD45⁺ cells comprising conventional CD3⁺ T cells (Tcons). In aspects and embodiments, a treatment and/or method further comprises a conditioning regimen, wherein the conditioning regimen is administered before administration of (a) one or more allogeneic cells comprising a chimeric receptor; and (b) an immune-modulating cell therapy product.

In embodiments, the conditioning regimen is a myeloablative conditioning regimen. In some cases, the conditioning regimen comprises at least three conditioning reagents, wherein at least one conditioning reagent is thiotepa. In various embodiments, the myeloablative conditioning regimen comprises at least one dose of thiotepa, e.g., at least about 5 milligrams thiotepa per kilogram of the human subject's actual or ideal body weight or at least about 10 milligrams thiotepa per kilogram of the human subject's actual or ideal body weight. In some embodiments, the conditioning regimen comprises one or more doses of busulfan, fludarabine and thiotepa. In embodiments, the one or more doses comprises from approximately 5 to approximately 12 mg of thiotepa per kg human subject's actual or ideal body weight, from approximately 7 to approximately 11 mg of busulfan per kg human subject actual or ideal body weight, and from approximately 100 to approximately 200 mg of fludarabine per meter² body surface area respectively.

In various embodiments, the method further comprises administering a myeloablative conditioning regimen to the patient prior to the administration of any cell population, the conditioning regimen comprising administration of at least one conditioning agent to the patient.

In some embodiments, the patient does not receive any irradiation as part of the myeloablative conditioning regimen.

In embodiments, the at least one conditioning agent is administered from approximately two to approximately ten days prior to the administration of any of the cell populations. In some cases, the at least one conditioning agent is administered about five days prior to the administration of any of the cell populations.

In various embodiments, the human subject has undergone myeloablative conditioning regimen before administration of any cell populations and the adverse event is associated with the myeloablative conditioning.

In some embodiments, the at least one conditioning agent comprises thiotepa. In some cases, a dose of thiotepa administered to the patient is in a range of from approximately 5 to approximately 10 mg per kilogram of actual or ideal body weight.

In embodiments, the at least one conditioning agent comprises busulfan and fludarabine. In some cases, doses of thiotepa, busulfan, and fludarabine administered to the patient comprise about 10 mg per kilogram of the patient's actual or ideal body weight, about 9.6 mg per kilogram of the patient's actual or ideal body weight, and approximately 150 mg per meter² body surface area respectively.

In some embodiments, the subject has been conditioned with radiation, chemotherapy, recombinant proteins, antibodies, or toxin-conjugated antibodies, or any combination thereof prior to administering a cell population or cell populations described herein. In some embodiments, the subject is conditioned for cellular graft therapy by first treating the subject with myeloablative therapy. Exemplary myeloablative therapies include chemotherapy or radiotherapy. Myeloablative therapies are thought to provide therapeutic benefit by debulking a tumor and/or reducing the number of cancer cells. Myeloablative regimens eradicate a sufficient number of HSCs that the patient would otherwise increase the chances of a patient developing GVHD. When HSPCs are subsequently administered to the myeloablated subject, the donor cells can further attack the cancer and/or and reconstitute the blood and the immune system of the subject.

In some embodiments, the myeloablative therapy comprises administration of thiotepa (TTP), busulfan, cyclophosphamide, Total Body Irradiation (TBI), Total Marrow and Lymphoid Irradiation (TMLI), fludarabine, etoposide, melphalan, anti-thymocyte globulin (ATG), or any combination thereof. In some embodiments, the myeloablative therapy comprises administration an anti-cKIT antibody. In some embodiments, the myeloablative therapy comprises administration an antibody drug conjugate. The antibody drug conjugate can be, for example, anti-CD45-saporin or anti-cKit-saporin therapeutic antibodies. In some embodiments, the myeloablative therapy is a reduced intensity conditioning therapy. Exemplary conditioning regimens are described in Table 15.

A conditioning regimen of this disclosure may comprise one or more doses of busulfan. A conditioning regimen of this disclosure may comprise fludarabine. A conditioning regimen of this disclosure may comprise one or more doses of Cyclophosphamide. A conditioning regimen of this disclosure may comprise one or more doses of Melphalan. A conditioning regimen of this disclosure may comprise one or more doses of Etoposide.

The methods of the disclosure can comprise administration of a combination of conditioning reagents prior to the administration of the cells. A conditioning regimen as described herein may comprise administering 1, 2, 3 or 4 different conditioning reagents. The conditioning reagents used herein may be alkylating agents. The conditioning reagents used herein may be myeloablative. The conditioning reagents used herein may be non-myeloablative. The conditioning reagents used herein may be myeloreductive. The conditioning reagents used herein may be a form of chemotherapy.

The conditioning regimen described herein may comprise administration of an alkylating agent such as thiotepa (TTP). A conditioning regimen of this disclosure comprising TTP may comprise at least one more conditioning reagent. The conditioning reagents administered to a subject in addition to TTP may comprise one or more reagents selected from busulfan, dimethyl myleran, prednisone, methyl prednisolone, azathioprine, cyclophosphamide, cyclosparine, monoclonal antibodies against T cells, antilymphocyte globulin and anti-thymocyte globulin, fludarabine, etoposide, radiation, total body irradiation (TBI), total marrow and lymphoid irradiation (TMLI). Aspects and embodiments include any combination of TTP with the one or more conditioning reagents. In some embodiments, a subject is administered a conditioning regimen comprising thiotepa, busulfan, and fludarabine. In some embodiments, a subject is administered a conditioning regimen comprising thiotepa, fludarabine, and TBI (e.g., HFTBI).

The conditioning regimen described herein may comprise administration of an alkylating agent such as TTP. In some cases, a conditioning regimen of the disclosure may comprise TTP administration on more than one day. A conditioning regimen of the disclosure may comprise administering 2 mg/kg to 14 mg/kg TTP to a subject. A conditioning regimen of this disclosure may comprise administering at least 3 mg/kg TTP to a subject. A conditioning regimen of this disclosure may comprise administering at most 14 mg/kg TTP to a subject. A conditioning regimen of this disclosure may comprise administering 2 mg/kg to 5 mg/kg, 2 mg/kg to 6 mg/kg, 2 mg/kg to 8 mg/kg, 2 mg/kg to 10 mg/kg, 2 mg/kg to 12 mg/kg, 2 mg/kg to 14 mg/kg, 5 mg/kg to 6 mg/kg, 5 mg/kg to 8 mg/kg, 5 mg/kg to 10 mg/kg, 5 mg/kg to 12 mg/kg, 5 mg/kg to 14 mg/kg, 6 mg/kg to 8 mg/kg, 6 mg/kg to 10 mg/kg, 6 mg/kg to 12 mg/kg, 6 mg/kg to 14 mg/kg, 8 mg/kg to 10 mg/kg, 8 mg/kg to 12 mg/kg, 8 mg/kg to 14 mg/kg, 10 mg/kg to 12 mg/kg, 10 mg/kg to 14 mg/kg, or 12 mg/kg to 14 mg/kg TTP to a subject. A conditioning regimen of this disclosure may comprise administering 2 mg/kg, 3 mg/kg, 4 mg/kg, 5 mg/kg, 6 mg/kg, 8 mg/kg, 10 mg/kg, 12 mg/kg, or 14 mg/kg TTP to a subject. A conditioning regimen of this disclosure may comprise administering at most 2 mg/kg, 3 mg/kg, 4 mg/kg, 5 mg/kg, 6 mg/kg, 8 mg/kg, 10 mg/kg, or 12 mg/kg TTP to a subject. A conditioning regimen of this disclosure may comprise administering at least 2 mg/kg, 3 mg/kg, 4 mg/kg, 5 mg/kg, 6 mg/kg, 8 mg/kg, 10 mg/kg, 12 mg/kg, or 14 mg/kg TTP to a subject.

As used herein, a recited dose, e.g., #mg/kg, may be relative to a subject's actual body weight (in kg) or relative to the subject's ideal body weight (in kg). Or the recited dose may be relative to a subject's adjusted body weight (ABW) if the subject's actual body weight is greater than 120% of the ideal body weight (IBW).

A subject administered one or more cell populations described herein may be administered one or more doses of TTP prior to the cell transplant. A subject receiving one or more cell populations described herein may be administered 1, 2, 3, 4, 5, 6, 7, 8, 9 or 10 doses of TTP prior to the cell transplant. In some cases, each dose of TTP has the same concentration. In some cases, one or more doses of TTP have different concentrations. A subject may be administered 1 mg/kg to 10 mg/kg TTP in a single dose. A subject may be administered at least 1 mg/kg TTP in a single dose. A subject may be administered at most 10 mg/kg TTP in a single dose. A subject may be administered 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 1 mg/kg to 10 mg/kg, 2 mg/kg to 3 mg/kg, 2 mg/kg to 4 mg/kg, 2 mg/kg to 5 mg/kg, 2 mg/kg to 6 mg/kg, 2 mg/kg to 7 mg/kg, 2 mg/kg to 8 mg/kg, 2 mg/kg to 9 mg/kg, 2 mg/kg to 10 mg/kg, 3 mg/kg to 4 mg/kg, 3 mg/kg to 5 mg/kg, 3 mg/kg to 6 mg/kg, 3 mg/kg to 7 mg/kg, 3 mg/kg to 8 mg/kg, 3 mg/kg to 9 mg/kg, 3 mg/kg to 10 mg/kg, 4 mg/kg to 5 mg/kg, 4 mg/kg to 6 mg/kg, 4 mg/kg to 7 mg/kg, 4 mg/kg to 8 mg/kg, 4 mg/kg to 9 mg/kg, 4 mg/kg to 10 mg/kg, 5 mg/kg to 6 mg/kg, 5 mg/kg to 7 mg/kg, 5 mg/kg to 8 mg/kg, 5 mg/kg to 9 mg/kg, 5 mg/kg to 10 mg/kg, 6 mg/kg to 7 mg/kg, 6 mg/kg to 8 mg/kg, 6 mg/kg to 9 mg/kg, 6 mg/kg to 10 mg/kg, 7 mg/kg to 8 mg/kg, 7 mg/kg to 9 mg/kg, 7 mg/kg to 10 mg/kg, 8 mg/kg to 9 mg/kg, 8 mg/kg to 10 mg/kg, or 9 mg/kg to 10 mg/kg TTP in a single dose. A subject may be administered 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose. A subject may be administered at most 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose. A subject may be administered at least 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose.

The methods of the disclosure can comprise administration of a combination of conditioning reagents prior to the administration of the cells. A conditioning regimen as described herein may comprise administering 1, 2, 3 or 4 different conditioning reagents. The conditioning reagents used herein may be alkylating agents. The conditioning reagents used herein may be myeloablative. The conditioning reagents used herein may be non-myeloablative. The conditioning reagents used herein may be myeloreductive. The conditioning reagents used herein may be a form of chemotherapy.

A conditioning regimen of this disclosure may comprise one or more doses of busulfan. One or more doses of busulfan may be administered to a subject before the administration of one or more doses of another conditioning reagent such as TTP. One or more doses of busulfan may be administered to a subject after the administration of one or more doses of another conditioning reagent such as TTP. One or more doses of busulfan may be administered to a subject along with the administration of one or more doses of another conditioning reagent such as TTP.

A conditioning regimen of this disclosure may comprise administering about 6 mg/kg to approximately 12 mg/kg busulfan to a subject. A conditioning regimen of this disclosure may comprise administering at least about 6 mg/kg busulfan to a subject. A conditioning regimen of this disclosure may comprise administering at most about 12 mg/kg busulfan to a subject. A conditioning regimen of this disclosure may comprise administering about 6 mg/kg to approximately 7 mg/kg, about 6 mg/kg to approximately 8 mg/kg, about 6 mg/kg to approximately 9 mg/kg, about 6 mg/kg to approximately 10 mg/kg, about 6 mg/kg to approximately 11 mg/kg, about 6 mg/kg to approximately 12 mg/kg, about 7 mg/kg to approximately 8 mg/kg, about 7 mg/kg to approximately 9 mg/kg, about 7 mg/kg to approximately 10 mg/kg, about 7 mg/kg to approximately 11 mg/kg, about 7 mg/kg to approximately 12 mg/kg, about 8 mg/kg to approximately 9 mg/kg, about 8 mg/kg to approximately 10 mg/kg, about 8 mg/kg to approximately 11 mg/kg, about 8 mg/kg to approximately 12 mg/kg, about 9 mg/kg to approximately 10 mg/kg, about 9 mg/kg to approximately 11 mg/kg, about 9 mg/kg to approximately 12 mg/kg, about 10 mg/kg to approximately 11 mg/kg, about 10 mg/kg to approximately 12 mg/kg, or about 11 mg/kg to approximately 12 mg/kg busulfan to a subject. A conditioning regimen of this disclosure may comprise administering 6 mg/kg, 7 mg/kg, 8 mg/kg, 9 mg/kg, 10 mg/kg, 11 mg/kg, or 12 mg/kg busulfan to a subject. A conditioning regimen of this disclosure may comprise administering at least 6 mg/kg, 7 mg/kg, 8 mg/kg, 9 mg/kg, 10 mg/kg, or 11 mg/kg busulfan to a subject. A conditioning regimen of this disclosure may comprise administering at most 7 mg/kg, 8 mg/kg, 9 mg/kg, 10 mg/kg, 11 mg/kg, or 12 mg/kg busulfan to a subject.

A subject receiving one or more cell populations described herein may be administered one or more doses of busulfan prior to the cell transplant. A subject receiving one or more cell components described herein may be administered 1, 2, 3, 4, 5, 6, 7, 8, 9 or 10 doses of busulfan prior to the cell transplant. In some cases, each dose of busulfan has the same concentration. In some cases, one or more doses of busulfan have different concentrations. A subject may be administered 1 mg/kg to 10 mg/kg busulfan in a single dose. A subject may be administered at least 1 mg/kg busulfan in a single dose. A subject may be administered at least 2 mg/kg busulfan in a single dose. A subject may be administered at least 3 mg/kg busulfan in a single dose.

A conditioning regimen of this disclosure may comprise one or more doses of fludarabine. One or more doses of fludarabine may be administered to a subject before the administration of one or more doses of another conditioning reagent such as TTP. One or more doses of fludarabine may be administered to a subject after the administration of one or more doses of another conditioning reagent such as TTP. One or more doses of fludarabine may be administered to a subject along with the administration of one or more doses of another conditioning reagent such as TTP.

A conditioning regimen of this disclosure may comprise administering 20 mg/m² to 180 mg/m² fludarabine to a subject based on the surface area of the subject. A conditioning regimen of this disclosure may comprise administering at least 20 mg/m² fludarabine to a subject. A conditioning regimen of this disclosure may comprise administering at most 180 mg/m² fludarabine to a subject. A conditioning regimen of this disclosure may comprise administering 20 mg/m² to 30 mg/m², 20 mg/m² to 40 mg/m², 20 mg/m² to 50 mg/m², 20 mg/m² to 60 mg/m², 20 mg/m² to 80 mg/m², 20 mg/m² to 100 mg/m², 20 mg/m² to 120 mg/m², 20 mg/m² to 150 mg/m², 20 mg/m² to 180 mg/m², 30 mg/m² to 40 mg/m², 30 mg/m² to 50 mg/m², 30 mg/m² to 60 mg/m², 30 mg/m² to 80 mg/m², 30 mg/m² to 100 mg/m², 30 mg/m² to 120 mg/m², 30 mg/m² to 150 mg/m², 30 mg/m² to 180 mg/m², 40 mg/m² to 50 mg/m², 40 mg/m² to 60 mg/m², 40 mg/m² to 80 mg/m², 40 mg/m² to 100 mg/m², 40 mg/m² to 120 mg/m², 40 mg/m² to 150 mg/m², 40 mg/m² to 180 mg/m², 50 mg/m² to 60 mg/m², 50 mg/m² to 80 mg/m², 50 mg/m² to 100 mg/m², 50 mg/m² to 120 mg/m², 50 mg/m² to 150 mg/m², 50 mg/m² to 180 mg/m², 60 mg/m² to 80 mg/m², 60 mg/m² to 100 mg/m², 60 mg/m² to 120 mg/m², 60 mg/m² to 150 mg/m², 60 mg/m² to 180 mg/m², 80 mg/m² to 100 mg/m², 80 mg/m² to 120 mg/m², 80 mg/m² to 150 mg/m², 80 mg/m² to 180 mg/m², 100 mg/m² to 120 mg/m², 100 mg/m² to 150 mg/m², 100 mg/m² to 180 mg/m², 120 mg/m² to 150 mg/m², 120 mg/m² to 180 mg/m², or 150 mg/m² to 180 mg/m² fludarabine to a subject. A conditioning regimen of this disclosure may comprise administering 20 mg/m², 30 mg/m², 40 mg/m², 50 mg/m², 60 mg/m², 80 mg/m², 100 mg/m², 120 mg/m², 150 mg/m², or 180 mg/m² fludarabine to a subject. A conditioning regimen of this disclosure may comprise administering at least 20 mg/m², 30 mg/m², 40 mg/m², 50 mg/m², 60 mg/m², 80 mg/m², 100 mg/m², 120 mg/m² or 150 mg/m² fludarabine to a subject. A conditioning regimen of this disclosure may comprise administering at most 30 mg/m², 40 mg/m², 50 mg/m², 60 mg/m², 80 mg/m², 100 mg/m², 120 mg/m², 150 mg/m², or 180 mg/m² fludarabine to a subject.

A subject receiving one or more cell components described herein may be administered one or more doses of fludarabine prior to the cell transplant. A subject receiving one or more cell components described herein may be administered 1, 2, 3, 4, 5, 6, 7, 8, 9 or 10 doses of fludarabine prior to the cell transplant. In some cases, each dose of fludarabine has the same concentration. In some cases, one or more doses of fludarabine have different concentrations. A subject may be administered 20 to 60 mg/m² dose of fludarabine in a single dose. A subject may be administered at least 30 mg/m² fludarabine in a single dose. A subject may be administered at least 40 mg/m² fludarabine in a single dose. A subject may be administered at least 50 mg/m² fludarabine in a single dose.

A conditioning regimen of this disclosure may comprise administering 20 mg/m² to 180 mg/m² melphalan to a subject based on the surface area of the subject. A conditioning regimen of this disclosure may comprise administering at least 20 mg/m² melphalan to a subject. A conditioning regimen of this disclosure may comprise administering at most 180 mg/m² melphalan to a subject. A conditioning regimen of this disclosure may comprise administering 20 mg/m² to 30 mg/m², 20 mg/m² to 40 mg/m², 20 mg/m² to 50 mg/m², 20 mg/m² to 60 mg/m², 20 mg/m² to 80 mg/m², 20 mg/m² to 100 mg/m², 20 mg/m² to 120 mg/m², 20 mg/m² to 150 mg/m², 20 mg/m² to 180 mg/m², 30 mg/m² to 40 mg/m², 30 mg/m² to 50 mg/m², 30 mg/m² to 60 mg/m², 30 mg/m² to 80 mg/m², 30 mg/m² to 100 mg/m², 30 mg/m² to 120 mg/m², 30 mg/m² to 150 mg/m², 30 mg/m² to 180 mg/m², 40 mg/m² to 50 mg/m², 40 mg/m² to 60 mg/m², 40 mg/m² to 80 mg/m², 40 mg/m² to 100 mg/m², 40 mg/m² to 120 mg/m², 40 mg/m² to 150 mg/m², 40 mg/m² to 180 mg/m², 50 mg/m² to 60 mg/m², 50 mg/m² to 80 mg/m², 50 mg/m² to 100 mg/m², 50 mg/m² to 120 mg/m², 50 mg/m² to 150 mg/m², 50 mg/m² to 180 mg/m², 60 mg/m² to 80 mg/m², 60 mg/m² to 100 mg/m², 60 mg/m² to 120 mg/m², 60 mg/m² to 150 mg/m², 60 mg/m² to 180 mg/m², 80 mg/m² to 100 mg/m², 80 mg/m² to 120 mg/m², 80 mg/m² to 150 mg/m², 80 mg/m² to 180 mg/m², 100 mg/m² to 120 mg/m², 100 mg/m² to 150 mg/m², 100 mg/m² to 180 mg/m², 120 mg/m² to 150 mg/m², 120 mg/m² to 180 mg/m², or 150 mg/m² to 180 mg/m² melphalan to a subject. A conditioning regimen of this disclosure may comprise administering 20 mg/m², 30 mg/m², 40 mg/m², 50 mg/m², 60 mg/m², 80 mg/m², 100 mg/m², 120 mg/m², 150 mg/m², or 180 mg/m² melphalan to a subject. A conditioning regimen of this disclosure may comprise administering at least 20 mg/m², 30 mg/m², 40 mg/m², 50 mg/m², 60 mg/m², 80 mg/m², 100 mg/m², 120 mg/m² or 150 mg/m² melphalan to a subject. A conditioning regimen of this disclosure may comprise administering at most 30 mg/m², 40 mg/m², 50 mg/m², 60 mg/m², 80 mg/m², 100 mg/m², 120 mg/m², 150 mg/m², or 180 mg/m² melphalan to a subject.

A subject receiving one or more cell components described herein may be administered one or more doses of melphalan prior to the cell transplant. A subject receiving one or more cell components described herein may be administered 1, 2, 3, 4, 5, 6, 7, 8, 9 or 10 doses of melphalan prior to the cell transplant. In some cases, each dose of melphalan has the same concentration. In some cases, one or more doses of melphalan have different concentrations.

A conditioning regimen of this disclosure may comprise administering 100 mg/kg to 140 mg/kg cyclophosphamide. A conditioning regimen of this disclosure may comprise administering at least 100 mg/kg cyclophosphamide. A conditioning regimen of this disclosure may comprise administering at most 140 mg/kg cyclophosphamide. A conditioning regimen of this disclosure may comprise administering 100 mg/kg to 110 mg/kg, 100 mg/kg to 120 mg/kg, 100 mg/kg to 130 mg/kg, 100 mg/kg to 140 mg/kg, 110 mg/kg to 120 mg/kg, 110 mg/kg to 130 mg/kg, 110 mg/kg to 140 mg/kg, 120 mg/kg to 130 mg/kg, 120 mg/kg to 140 mg/kg, or 130 mg/kg to 140 mg/kg cyclophosphamide. A conditioning regimen of this disclosure may comprise administering about 100 mg/kg, 110 mg/kg, 120 mg/kg, 130 mg/kg, or 140 mg/kg cyclophosphamide. A conditioning regimen of this disclosure may comprise administering at least 100 mg/kg, 110 mg/kg, 120 mg/kg, or 130 mg/kg cyclophosphamide. A conditioning regimen of this disclosure may comprise administering at most 110 mg/kg, 120 mg/kg, 130 mg/kg, or 140 mg/kg cyclophosphamide.

A subject receiving one or more cell components described herein may be administered one or more doses of cyclophosphamide prior to the cell transplant. A subject receiving one or more cell components described herein may be administered 1, 2, 3, 4, 5, 6, 7, 8, 9 or 10 doses of cyclophosphamide prior to the cell transplant. In some cases, each dose of cyclophosphamide has the same concentration. In some cases, one or more doses of cyclophosphamide have different concentrations.

A conditioning regimen of this disclosure may comprise administering 40 mg/kg to 80 mg/kg etoposide. A conditioning regimen of this disclosure may comprise administering at least 40 mg/kg etoposide. A conditioning regimen of this disclosure may comprise administering at most 80 mg/kg etoposide. A conditioning regimen of this disclosure may comprise administering 40 mg/kg to 50 mg/kg, 40 mg/kg to 60 mg/kg, 40 mg/kg to 70 mg/kg, 40 mg/kg to 80 mg/kg, 50 mg/kg to 60 mg/kg, 50 mg/kg to 70 mg/kg, 50 mg/kg to 80 mg/kg, 60 mg/kg to 70 mg/kg, 60 mg/kg to 80 mg/kg, or 70 mg/kg to 80 mg/kg etoposide. A conditioning regimen of this disclosure may comprise administering about 40 mg/kg, 50 mg/kg, 60 mg/kg, 70 mg/kg, or 80 mg/kg etoposide. A conditioning regimen of this disclosure may comprise administering at least 40 mg/kg, 50 mg/kg, 60 mg/kg, or 70 mg/kg etoposide. A conditioning regimen of this disclosure may comprise administering at most 50 mg/kg, 60 mg/kg, 70 mg/kg, or 80 mg/kg etoposide.

A subject receiving one or more cell components described herein may be administered one or more doses of etoposide prior to the cell transplant. A subject receiving one or more cell components described herein may be administered 1, 2, 3, 4, 5, 6, 7, 8, 9 or 10 doses of etoposide prior to the cell transplant. In some cases, each dose of etoposide has the same concentration. In some cases, one or more doses of etoposide have different concentrations.

A conditioning regimen of this disclosure comprising TTP may comprise one or more doses of total body irradiation (TBI), such as fractionated total body irradiation (fTBI) or hyperfractionated TBI (HFTBI). One or more doses of fTBI or HFTBI may be administered to a subject before the administration of one or more doses of another conditioning reagent such as TTP. One or more doses of fTBI or HFTBI may be administered to a subject after the administration of one or more doses of another conditioning reagent such as TTP. One or more doses of fTBI or HFTBI may be administered to a subject along with the administration of one or more doses of another conditioning reagent such as TTP.

A conditioning regimen of this disclosure may comprise administering 800 cGy to 1,500 cGy fTBI or HFTBI to a subject. A conditioning regimen of this disclosure may comprise administering at least 800 cGy fTBI or HFTBI to a subject. A conditioning regimen of this disclosure may comprise administering at most 1,500 cGy fTBI or HFTBI to a subject. A conditioning regimen of this disclosure may comprise administering 800 cGy to 900 cGy, 800 cGy to 1,000 cGy, 800 cGy to 1,100 cGy, 800 cGy to 1,200 cGy, 800 cGy to 1,300 cGy, 800 cGy to 1,375 cGy, 800 cGy to 1,400 cGy, 800 cGy to 1,500 cGy, 900 cGy to 1,000 cGy, 900 cGy to 1,100 cGy, 900 cGy to 1,200 cGy, 900 cGy to 1,300 cGy, 900 cGy to 1,375 cGy, 900 cGy to 1,400 cGy, 900 cGy to 1,500 cGy, 1,000 cGy to 1,100 cGy, 1,000 cGy to 1,200 cGy, 1,000 cGy to 1,300 cGy, 1,000 cGy to 1,375 cGy, 1,000 cGy to 1,400 cGy, 1,000 cGy to 1,500 cGy, 1,100 cGy to 1,200 cGy, 1,100 cGy to 1,300 cGy, 1,100 cGy to 1,375 cGy, 1,100 cGy to 1,400 cGy, 1,100 cGy to 1,500 cGy, 1,200 cGy to 1,300 cGy, 1,200 cGy to 1,375 cGy, 1,200 cGy to 1,400 cGy, 1,200 cGy to 1,500 cGy, 1,300 cGy to 1,375 cGy, 1,300 cGy to 1,400 cGy, 1,300 cGy to 1,500 cGy, 1,375 cGy to 1,400 cGy, 1,375 cGy to 1,500 cGy, or 1,400 cGy to 1,500 cGy fTBI or HFTBI to a subject. A conditioning regimen of this disclosure may comprise administering about 800 cGy, 900 cGy, 1,000 cGy, 1,100 cGy, 1,200 cGy, 1,300 cGy, 1,375 cGy, 1,400 cGy, or 1,500 cGy fTBI or HFTBI to a subject. A conditioning regimen of this disclosure may comprise administering at least 800 cGy, 900 cGy, 1,000 cGy, 1,100 cGy, 1,200 cGy, 1,300 cGy, 1,375 cGy or 1,400 cGy fTBI or HFTBI to a subject. A conditioning regimen of this disclosure may comprise administering at most 900 cGy, 1,000 cGy, 1,100 cGy, 1,200 cGy, 1,300 cGy, 1,375 cGy, 1,400 cGy, or 1,500 cGy fTBI or HFTBI to a subject.

A subject receiving one or more cell components described herein may be administered one or more doses of fTBI or HFTBI prior to the cell transplant. A subject receiving one or more cell components described herein may be administered 1, 2, 3, 4, 5, 6, 7, 8, 9 or 10 doses of fTBI or HFTBI prior to the cell transplant. In some cases, each dose of fTBI or HFTBI has the same concentration. In some cases, one or more doses of fTBI or HFTBI have different concentrations. A subject may be administered 75 to 150 cGys of fTBI or HFTBI in a single dose. A subject may be administered at least 75 cGys fTBI or HFTBI in a single dose. A subject may be administered at least 100 cGys fTBI or HFTBI in a single dose. A subject may be administered at least 125 cGys fTBI or HFTBI in a single dose.

A conditioning regimen of this disclosure may comprise administering low-dose TBI. In some embodiments, a subject may be administered 1, 2, 3, 4, 5, 6, 7, 8, 9 or 10 doses of low-dose TBI. In some embodiments, a subject may be administered 1 to 20 cGys of TBI in a single dose. A conditioning regimen of this disclosure may comprise administering about 1 cGys, about 2 cGys, about 3 cGys, about 4 cGys, about 5 cGys, about 6 cGys, about 7 cGys, about 8 cGys, about 9 cGys, about 10 cGys, about 11 cGys, about 12 cGys, about 13 cGys, about 14 cGys, about 15 cGys, about 16 cGys, about 17 cGys, about 18 cGys, about 19 cGys, about 20 cGys, about 25 cGys, about 30 cGys, about 35 cGys, about 40 cGys, about 45 cGys, about 50 cGys, about 55 cGys, about 60 cGys, about 65 cGys, about 70 cGys, about 75 cGys, about 80 cGys, about 85 cGys, about 90 cGys, about 100 cGys, about 125 cGys, about 150 cGys, about 175 cGys, about 200 cGys, about 225 cGys, about 250 cGys, about 275 cGys, about 300 cGys, about 325 cGys, about 350 cGys, about 375 cGys, about 400 cGys, about 425 cGys, about 450 cGys, about 475 cGys, about 500 cGys, about 525 cGys, about 550 cGys, about 575 cGys, about 600 cGys, about 625 cGys, about 650 cGys, about 675 cGys, about 700 cGys, about 725 cGys, about 750 cGys, about 775 cGys, about 800 cGys, about 825 cGys, about 850 cGys, about 875 cGys, about 900 cGys, about 925 cGys, about 950 cGys, about 975 cGys, or about 1000 cGys of TBI in a single dose to a subject.

Exemplary Conditioning Regimen 1: In some embodiments, a subject is administered a conditioning regimen comprising thiotepa, busulfan, and fludarabine. In some embodiments, a subject is administered thiotepa at 5 mg/kg actual body weight or ideal body weight. In some embodiments, a subject is administered thiotepa at approximately 5 mg/kg for two days (e.g., consecutive days). In some embodiments, a subject is administered busulfan at approximately 3.2 mg/kg actual body weight or ideal body weight. In some embodiments, a subject is administered busulfan at approximately 3.2 mg/kg daily for three days (e.g., consecutive days). In some embodiments, a subject is administered fludarabine at approximately 50 mg/m². (meters squared-body surface area). In some embodiments, a subject is administered fludarabine at approximately 50 mg/m² for three days (e.g., consecutive days). In some embodiments, a subject is administered thiotepa at 5 mg/kg actual body weight or ideal body weight, is administered busulfan at approximately 3.2 mg/kg actual body weight or ideal body weight, and is administered fludarabine at approximately 50 mg/m2. (meters squared-body surface area). In some embodiments, a subject is administered thiotepa at approximately 5 mg/kg for two days (e.g., consecutive days), is administered busulfan at approximately 3.2 mg/kg daily for three days (e.g., consecutive days), and is administered fludarabine at approximately 50 mg/m² for three days (e.g., consecutive days). In some embodiments, a subject is administered thiotepa at approximately 5 mg/kg on days −7 and −6, is administered busulfan at approximately 3.2 mg/kg daily on days −5 to −3, and is administered fludarabine at approximately 50 mg/m² on days −5 to −3.

Exemplary Conditioning Regimen 2: In some embodiments, a subject is administered a conditioning regimen comprising thiotepa, fludarabine, and TBI (e.g., HFTBI). In some embodiments, a subject is administered thiotepa at 5 mg/kg actual body weight or ideal body weight. In some embodiments, a subject is administered thiotepa at approximately 5 mg/kg for two days (e.g., consecutive days). In some embodiments, a subject is administered fludarabine at approximately 25 mg/m². (meters squared-body surface area). In some embodiments, a subject is administered fludarabine at approximately 25 mg/m² for three days (e.g., consecutive days). In some embodiments, a subject is administered HFTBI of 125 cGy (centigray). In some embodiments, a subject is administered HFTBI in 11 fractions of 125 cGy. In some embodiments, a subject is administered HFTBI in 11 fractions of 125 cGy over 4 days. In some embodiments, a subject is administered thiotepa at 5 mg/kg actual body weight or ideal body weight, is administered fludarabine at approximately 25 mg/m². (meters squared-body surface area), and is administered HFTBI of 125 cGy. In some embodiments, a subject is administered thiotepa at approximately 5 mg/kg for two days (e.g., consecutive days), is administered fludarabine at approximately 50 mg/m² for three days (e.g., consecutive days), and is administered HFTBI in 11 fractions of 125 cGy. In some embodiments, a subject is administered thiotepa at approximately 5 mg/kg on days −7 and −6, is administered fludarabine at approximately 50 mg/m² on days −5 to −3, and is administered HFTBI in 11 fractions of 125 cGy over 4 days.

A subject may receive the one or more cell populations described herein 1 day after completing a conditioning regimen. A subject may receive the one or more cell components described herein 2, 3, 4, 5, 6, 7, 8, 9, 10 days after completing a conditioning regimen. A subject may receive the one or more cell components described herein 1 day after receiving a final dose of a conditioning reagent such as TTP. A subject may receive the one or more cell components described herein 2, 3, 4, 5, 6, 7, 8, 9 or 10 days after receiving a final dose of a conditioning reagent such as TTP. A subject may receive the one or more cell components described herein 1 day after receiving a first dose of a conditioning reagent such as TTP. A subject may receive the one or more cell components described herein 2, 3, 4, 5, 6, 7, 8, 9 or 10 days after receiving a first dose of a conditioning reagent such as TTP.

### Graft Versus Host Disease (GVHD)

Graft versus host disease (GVHD) is a significant cause of morbidity and mortality in hematopoietic stem cell transplantation (HCT) recipients and/or may be a cause of morbidity and mortality in recipients of allogeneic cells comprising a chimeric receptor (e.g., a TCR or a CAR-T). Aspects and embodiments herein provide compositions and methods that reduce the incidence of GVHD, reduce the severity of GVHD, reduce the relative risk of GVHD, prevent GVHD, or a combination thereof in HCT recipients.

Graft-versus-host disease (GVHD) is an inflammatory disease that can occur in the allogeneic transplant setting. GVHD involves donor cells (graft) attacking recipient cells (host). GVHD can be life-threatening and can involve, for example, the skin, the intestines, and/or the liver. The morbidity and mortality associated GVHD can be a major factor limiting the success of HCT. GVHD can occur despite use of an HLA-matched sibling donor, and the use of various GVHD prophylactic/immunosuppressive agents, for example, use of two or more of tacrolimus, sirolimus, cyclosporine, methotrexate, mycophenolate, mycophenolate mofetil (MMF), anti-thymocyte globulin and corticosteroids.

In some embodiments, a multi-component pharmaceutical treatment or a multi-component cellular therapy product of the present disclosure further comprises from approximately 100 mg to approximately 5000 mg of mycophenolate mofetil (MMF). In some embodiments, a method of the present disclosure further comprises administering or otherwise providing from approximately 100 mg to approximately 5000 mg of mycophenolate mofetil (MMF). In some embodiments, the MMF is provided an amount or dose that is approximately 100 mg, approximately 150 mg, approximately 200 mg, approximately 250 mg, approximately 300 mg, approximately 350 mg, approximately 400 mg, approximately 450 mg, approximately 500 mg, approximately 550 mg, approximately 600 mg, approximately 650 mg, approximately 700 mg, approximately 750 mg, approximately 800 mg, approximately 850 mg, approximately 900 mg, approximately 950 mg, approximately 1000 mg, approximately 1050 mg, approximately 1100 mg, approximately 1150 mg, approximately 1200 mg, approximately 1250 mg, approximately 1300 mg, approximately 1350 mg, approximately 1400 mg, approximately 1450 mg, approximately 1500 mg, approximately 1550 mg, approximately 1600 mg, approximately 1650 mg, approximately 1700 mg, approximately 1750 mg, approximately 1800 mg, approximately 1850 mg, approximately 1900 mg, approximately 1950 mg, approximately 2000 mg, approximately 2050 mg, approximately 2100, approximately 2150 mg, approximately 2200 mg, approximately 2250 mg, approximately 2300 mg, approximately 2350 mg, approximately 2400 mg, approximately 2450 mg, approximately 2500 mg, approximately 2550 mg, approximately 2600 mg, approximately 2650 mg, approximately 2700 mg, approximately 2750 mg, approximately 2800 mg, approximately 2850 mg, approximately 2900 mg, approximately 2950 mg, approximately 3000 mg, approximately 3050 mg, approximately 3100, approximately 3150 mg, approximately 3200 mg, approximately 3250 mg, approximately 3300 mg, approximately 3350 mg, approximately 3400 mg, approximately 3450 mg, approximately 3500 mg, approximately 3550 mg, approximately 3600 mg, approximately 3650 mg, approximately 3700 mg, approximately 3750 mg, approximately 3800 mg, approximately 3850 mg, approximately 3900 mg, approximately 3950 mg, approximately 4000 mg, approximately 4050 mg, approximately 4100, approximately 4150 mg, approximately 4200 mg, approximately 4250 mg, approximately 4300 mg, approximately 4350 mg, approximately 4400 mg, approximately 4450 mg, approximately 4500 mg, approximately 4550 mg, approximately 4600 mg, approximately 4650 mg, approximately 4700 mg, approximately 4750 mg, approximately 4800 mg, approximately 4850 mg, approximately 4900 mg, approximately 4950 mg, or approximately 5000 mg.

### GVHD Classification and Grading

GVHD can be classified into acute GVHD (aGVHD) and chronic GVHD (cGVHD). In some embodiments, GVHD that occurs within the first 100 days post-transplant can be referred to as aGVHD, and GVHD after the first 100 days can be referred to as chronic GVHD (cGVHD). cGVHD is a major source of late treatment-related complications, and can be life-threatening. In addition to inflammation, cGVHD can lead to the development of fibrosis, which can result in functional disability.

Yet another aspect provides any herein-disclosed multi-component pharmaceutical treatment in which a risk and/or severity of an adverse event associated with the multi-component pharmaceutical treatment is reduced as compared to a similar pharmaceutical treatment in which a human subject receives Tcons but does not receive Tregs or is any herein-disclosed method in which a risk and/or severity of an adverse event associated with the method is reduced as compared to a similar method in which a human subject receives Tcons but does not receive Tregs.

In various embodiments, the adverse event is acute GVHD (aGVHD),

In some embodiments, the adverse event is stage two or greater aGVHD.

In embodiments, the adverse event is chronic GVHD (cGVHD).

In various embodiments, the human subject has no cGVHD about one year after being administered the cell populations.

In some embodiments, the adverse event is moderate to severe cGVHD.

In embodiments, the adverse event is acute graft vs host disease (aGVHD), e.g., stage two or greater aGVHD. In some cases, the patient has no stage two or higher aGVHD about 180 days after being administered the cell populations.

In various embodiments, the adverse event is chronic graft vs host disease (cGVHD). In some cases, the patient has no cGVHD about one year after being administered the cell populations.

In some embodiments, the adverse event is moderate to severe cGVHD. In some cases, the patient does not have moderate to severe cGVHD about one year after being administered the cell populations.

In embodiments, a patient does not develop GVHD within about 30 days of administration of the Tcons, does not develop GVHD within about 100 days of administration of the Tcons, does not develop GVHD within about 180 days of administration of the Tcons, and/or does not develop GVHD within about one year of administration of the Tcons.

In various embodiments, a human subject does not develop higher than stage 2 GVHD within about 100 days of the administering of the third population of CD45⁺ cells, the human subject does not develop higher than stage 2 GVHD) within about 180 days or within about 200 days of the administering of the third population of CD45⁺ cells, the human subject does not develop higher than stage 2 GVHD within about 1 year of the administering of the third population of CD45⁺ cells.

aGVHD and cGVHD can be graded using a system that first evaluates GVHD stages for the skin, liver, and gut, and then combines scoring from the organ staging to determine an overall GVHD grade. An example of a GVHD staging criteria that can be used for individual organs are provided in TABLE 4:

TABLE 4

| Organ | Stage | Description |
|---|---|---|
| Skin | 0 | No active (erythematous) GVHD rash |
| | 1 | Maculo-papular eruption over <25% of body area |
| | 2 | Maculo-papular eruption over >25 to 50% of body area |
| | 3 | Generalized erythroderma/maculo-papular eruption |
| | | over >50% of body area |
| | 4 | Generalized erythroderma/maculo-papular eruption |
| | | over >50% of body area plus bullous formation |
| | | and desquamation over >5% of body area |
| Liver | 0 | Bilirubin <2 mg/dL |
| | 1 | Bilirubin 2.0 to 3.0 mg/dL; serum glutamic oxaloacetic |
| | | transaminase (SGOT) SGOT 150 to 750IU |
| | 2 | Bilirubin 3.1 to 6.0 mg/dL |
| | 3 | Bilirubin 6.1 to 15.0 mg/dL |
| | 4 | Bilirubin >15.0 mg/dL |
| Gut | 0 | <500 mL/day or <3 episodes/day |
| | 1 | Diarrhea >30 mL/kg or >500 mL/day |
| | 2 | Diarrhea >60 mL/kg or >1000 mL/day |
| | 3 | Diarrhea >90 mL/kg or >1500 mL/day |
| | 4 | Diarrhea >90 mL/kg or >2000 mL/day; or severe |
| | | abdominal pain with or without ileus |

TABLE 5 provides one set of criteria for assessing overall GVHD grade.

TABLE 5

| | | | | | ECOG |
|---|---|---|---|---|---|
| Grade | Skin | Liver | | Gut | performance |
| 1 | 1 to 2 | 0 | | 0 | 0 |
| 2 | 1 to 3 | 1 | and/or | 1 | 0-1 |
| 3 | 2 to 3 | 2 to 4 | and/or | 2 to 3 | 2-3 |
| 4 | 2 to 4 | 2 to 4 | and/or | 2 to 3 | 3-4 |

aGVHD grade can also be determined based on most severe target organ involvement as defined in the MAGIC standardization criteria described by Harris et al., “International, multicenter standardization of acute graft-versus-host disease clinical data collection: a report from the Mount Sinai Acute GVHD International Consortium.” Biology of Blood and Marrow Transplantation 22.1 (2016): 4-10. For example, aGVHD organ staging can be evaluated according to TABLE 6, and overall aGVHD grade can be assessed as follows:
Grade 0: No Stage 1-4 of any organ.
Grade I: Stage 1-2 skin without liver, upper GI, or lower GI involvement.
Grade II: Stage 3 rash and/or Stage 1 liver and/or Stage 1 upper GI and/or Stage 1 lower GI.
Grade III: Stage 2-3 liver and/or Stage 2-3 lower GI, with Stage 0-3 skin and/or Stage 0-1 upper GI. Grade IV: Stage 4 skin, liver, or lower GI involvement, with Stage 0-1 upper GI.

TABLE 6

| | Skin (Active | Liver | | | Lower GI (stool |
|---|---|---|---|---|---|
| Stage | Erythema Only) | (Bilirubin) | | Upper GI | output/day) |
| 0 | No active | <2 | mg/dL | No or intermittent | Adult: <500 mL/day |
| | (erythematous) | | | nausea, vomiting | or <3 episodes/day |
| | GvHD rash | | | or anorexia | Child: <10 mL/kg/day |
| | | | | | or <4 episodes/day |
| 1 | Maculopapular | 2-3 | mg/dL | Persistent nausea, | Adult: 500-999 mL/day or 3-4 |
| | rash <25% | | | vomiting or | episodes/day |
| | BSA | | | anorexia | Child: 10-19.9 mL/kg/day or 5-7 |
| | | | | | episodes/day |
| 2 | Maculopapular | 3.1-6 | mg/dL | | Adult: 1000-1500 mL/day or 57 |
| | rash 25-50% | | | | episodes/day |
| | BSA | | | | Child: 20-30 mL/kg/day or 7- |
| | | | | | 10 episodes/day |
| 3 | Maculopapular | 6.1-15 | mg/dL | | Adult: >1500 mL/day or >7 |
| | rash >50% | | | | episodes/day |
| | BSA | | | | Child: >30 ml/kg/day or >10 |
| | | | | | episodes/day |
| 4 | Generalized | >15 | mg/dL | | Severe abdominal pain with or |

| | |
|---|---|
| erythroderma | without ileus or grossly bloody |
| (>50% BSA) plus | stool (regardless of stool volume) |
| bullous formation | |
| and desquamation | |
| >5% BSA | |

In some embodiments, GVHD stage and GVHD grade are synonyms.

cGVHD can also be assessed by the method described by Jagasia et al., “National Institutes of Health consensus development project on criteria for clinical trials in chronic graft-versus-host disease: I. The 2014 diagnosis and staging working group report.” Biology of Blood and Marrow Transplantation 21.3 (2015): 389-401. To establish a diagnosis of cGVHD, these criteria can require, for example, at least one diagnostic manifestation of chronic GVHD or at least one distinctive manifestation plus a pertinent biopsy, laboratory, or other test (e.g., PFTs, Schirmer's test), evaluation by a specialist (ophthalmologist, gynecologist) or radiographic imaging showing chronic GVHD in the same or another organ.

Organ systems can be scored as described in Jagasia et al., and Mild cGVHD can be present when one or two organs are involved with no more than score 1, plus a lung score of zero; moderate cGVHD can be present when three or more organs are involved with no more than score 1, or when at least one non-lung organ has a score of 2, or when the lungs have a score of 1; and severe cGVHD can be present when at least one organ has a score of 4, or the lungs have a score of 2 or 3.

TABLE 7 provides diagnostic and distinctive manifestations of cGVHD. infection, drug effect, malignancy, or other causes are excluded for distinctive manifestations. Bronchiolitis obliterans syndrome can be diagnostic for lung chronic GVHD only if distinctive sign or symptom present in another organ. Diagnosis of chronic GVHD based on myositis or polymyositis can require a biopsy.

TABLE 7

| | Diagnostic (Sufficient to | Distinctive (Seen in chronic |
|---|---|---|
| Organ or | Establish the Diagnosis | GVHD, but Insufficient Alone |
| Site | of chronic GVHD) | to Establish a Diagnosis) |
| Skin | Poikiloderma Lichen | Depigmentation Papulosquamous |
| | planus-like features | lesions |
| | Sclerotic features Morphea- | |
| | like features | |
| | Lichen sclerosus-like | |
| | features | |
| Nails | | Dystrophy |
| | | Longitudinal ridging, splitting or brittle features |
| | | Onycholysis |
| | | Pterygium unguis |
| | | Nail loss (usually symmetric, affects most nails) |
| Scalp and | | New onset of scarring or nonscarring scalp |
| body hair | | alopecia (after recovery from |
| | | chemoradiotherapy) |
| | | Loss of body hair |
| Mouth | Lichen planus-like changes | Xerostomia Mucoceles |
| | | Mucosal atrophy Ulcers |
| | | Pseudomembranes |
| Eyes | | New onset dry, gritty, or |
| | | painful eyes |
| | | Cicatricial conjunctivitis |
| | | Keratoconjunctivitis sicca |
| | | Confluent areas of punctate |
| | | keratopathy |
| Genitalia | Lichen planus-like features Lichen | Erosions Fissures |
| | sclerosus-like features | |
| Females | Vaginal scarring or clitoral/labial | Ulcers |
| | agglutination | |
| Males | Phimosis or urethral/meatus | |
| | scarring or stenosis | |
| GI Tract | Esophageal web Strictures or | |
| | stenosis in the upper to | |
| | mid third of the esophagus | |
| Lung | Bronchiolitis obliterans diagnosed | Air trapping and bronchiectasis |
| | with lung biopsy | on chest CT |
| | Bronchiolitis obliterans syndrome | |
| Muscles, | Joint stiffness or contractures | Myositis or polymyositis |
| fascia, joints | secondary to fasciitis or sclerosis | |

In some embodiments, GVHD severity can be graded using the Glucksberg grade (I-IV) or the International Bone Marrow Transplant Registry (IBMTR) grading system (A-D). The severity of acute GVHD can be determined by an assessment of the degree of involvement of the skin, liver, and gastrointestinal tract. The stages of individual organ involvement are combined with (Glucksberg) or without (IBMTR) the patient's performance status to produce an overall grade.

### GVHD Prophylaxis and Treatment

Immunosuppressive agents can be used to reduce the likelihood of GVHD (GVHD prophylactic agents), or to treat GVHD once it occurs (GVHD therapeutic agents). However, in some cases, the use of GVHD prophylactic agents, GVHD therapeutic agents, or both can be insufficient to effectively prevent or treat GVHD. For example, the incidence of GVHD in graft recipients can be high despite use of use of tacrolimus, sirolimus, cyclosporine, methotrexate, mycophenolate, anti-thymocyte globulin, corticosteroids, or a combination thereof (e.g., two or more of the agents).

Administering multiple GVHD prophylactic and/or therapeutic agents, high doses of GVHD prophylactic and/or therapeutic agents, or both can fail to effectively treat GVHD in many alloHSCT settings or can result in increased susceptibility to infection and decreased graft versus tumor therapeutic effects.

Non-limiting examples of GVHD prophylactic and/or GVHD therapeutic agents that can be used include calcineurin inhibitors (e.g., tacrolimus, cyclosporine A), sirolimus, monoclonal antibodies, methotrexate, mycophenolate, anti-thymocyte globulin, corticosteroids, azathioprine, and mycophenolate mofetil. Monoclonal antibodies useful as immunosuppressive agents include, for example, antagonist antibodies, (e.g., antibodies that antagonize IL-2R such as basiliximab and daclizumab), and antibodies that deplete an immune cell population by antibody dependent cellular cytotoxicity (e.g., anti-CD3 antibodies for T cell depletion such as muromonab-CD3).

Compositions and methods described herein may comprise administering one or more GVHD prophylactic agents to an HCT recipient. GVHD prophylaxis in such cases should be considered different from GVHD treatment such that the GVHD prophylactic agent will be administered to the HCT recipient before an incidence of GVHD is assessed. In some cases, an HCT recipient may be administered one or more GVHD prophylactic agents but not a GVHD therapeutic agent. In some cases, a HCT recipient does not require treatment for GVHD and/or does not receive treatment for GVHD.

Compositions and methods disclosed herein can reduce the incidence of GVHD, reduce the severity of GVHD, reduce the relative risk of GVHD, prevent GVHD, or a combination thereof in HCT recipients. In some embodiments, such benefits are achieved despite administering no GVHD prophylactic agents as disclosed herein. In some embodiments, such benefits are achieved despite administering a reduced number of GVHD prophylactic agents (e.g., a single GVHD prophylactic agents), a low dose of GVHD prophylactic agent(s), or a combination thereof as disclosed herein. In some embodiments, 1 GVHD prophylactic agent is administered to a subject. In some embodiments, no more than GVHD prophylactic agent is administered to a subject. In some embodiments, 2 GVHD prophylactic agents are administered to a subject. In some embodiments, no more than 2 GVHD prophylactic agents are administered to a subject.

In some embodiments, one or more GVHD prophylactic agents may be administered to a HCT recipient for a duration of 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 18 months, 24 months, 36 months post-transplant of one or more cell populations. One or more GVHD prophylactic agents may be administered to an HCT recipient starting the day of transplant of one or more cell populations. For instance, a GVHD prophylactic regimen may begin the day an HSPC cell population and/or a Treg cell population is administered to the recipient. Alternatively, a GVHD prophylactic regimen may begin the day a Tcon cell population is administered to the patient.

In some embodiments, tacrolimus is not administered to a subject. In some embodiments, sirolimus is not administered to a subject. In some embodiments, cyclosporine is not administered to a subject. In some embodiments, methotrexate is not administered to a subject. In some embodiments, mycophenolate is not administered to a subject. In some embodiments, anti-thymocyte globulin is not administered to a subject. In some embodiments, corticosteroids are not administered to a subject.

In some embodiments, the GVHD prophylactic agent is tacrolimus.

In embodiments, the tacrolimus graft versus host disease (GVHD) prophylactic agent (GVHDPA) is intravenously administered or orally administered. In various embodiments, administration of the tacrolimus graft versus host disease (GVHD) prophylactic agent (GVHDPA) is started from approximately 12 to approximately 24 hours after administration of the T-cons. In some cases, the tacrolimus GHVDPA is administered for a period of time up to approximately 90 days, is administered for a period of time up to approximately 60 days. In some embodiments, the tacrolimus GHVDPA is initially administered to the patient at approximately 0.03 mg/kg patient's actual or ideal body weight/day. In some cases, a dose of the tacrolimus GVHDPA administered to the patient is tapered starting at approximately 90 days after a first dose is administered to the patient or is tapered starting at approximately 45 days after a first dose is administered to the patient.

Aspects and embodiments herein provide a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending the cells, wherein at least about 30% of the lymphocyte comprise conventional T cells (Tcons); and administering to the patient over a period of time up to approximately 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

In cases where aGVHD occurs, responsiveness of aGVHD to GVHD therapeutic agents (e.g., corticosteroids) can be assessed by the criteria of TABLE 8.

TABLE 8

| | |
|---|---|
| Complete response | Complete resolution of acute GvHD symptoms in all |
| (CR) | organs, without secondary GvHD therapy |
| Partial | Improvement in GvHD stage in all initial GvHD |
| response (PR) | target organs without complete resolution and |
| | without worsening in any other GvHD target |
| | organs, without secondary GvHD therapy |
| Very good partial | Improvement in GvHD in all initial GvHD target |
| response (VGPR) | organs, with maximum Stage I involvement in |
| | one or more organs (except upper gastrointestinal |
| | tract), without secondary GvHD therapy |
| No response | Same grade of GvHD or progression of GvHD in |
| (NR) | any organ or death, or the addition of secondary |
| | GvHD therapy |
| Progression | Worsening GvHD in at least 1 organ with or without |
| | amelioration in any organ |

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a complete response to GVHD therapeutic agents.

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a partial response to GVHD therapeutic agents.

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a very good partial response to GVHD therapeutic agents.

In cases where cGVHD occurs, responsiveness of cGVHD to GVHD therapeutic agents (e.g., corticosteroids) can be assessed by the criteria of TABLE 9. Abbreviations used: ALT, alanine transaminase; FEV1, forced expiratory volume in the first second; OMRS, Oral Mucosa Rating Scale; PFTs, pulmonary function tests; P-ROM, photographic range of motion; ULN, upper limit of normal.

TABLE 9

| Organ | Complete Response | Partial Response | Progression |
|---|---|---|---|
| Skin | NIH Skin Score 0 after | Decrease in NIH Skin | Increase in NIH Skin Score by |
| | previous involvement | Score by 1 or more points | 1 or more points, except 0 to 1 |
| Eyes | NIH Eye Score 0 after | Decrease in NIH Eye | Increase in NIH Eye Score by 1 |
| | previous involvement | Score by 1 or more points | or more points, except 0 to 1 |
| Mouth | NIH Modified OMRS 0 | Decrease in NIH Modified | Increase in NIH Modified |
| | after previous involvement | OMRS of 2 or more points | OMRS of 2 or more points |
| Esophagus | NIH Esophagus Score 0 | Decrease in NIH | Increase in NIH Esophagus |
| | after previous involvement | Esophagus Score by 1 or | Score by 1 or more points, |
| | | more points | except 0 to 1 |
| Upper GI | NIH Upper GI Score 0 after | Decrease in NIH Upper | Increase in NIH Upper GI |
| | previous involvement | GI Score by 1 or more | Score by 1 or more points, |
| | | points | except 0 to 1 |
| Lower GI | NIH Lower GI Score 0 after | Decrease in NIH Lower | Increase in NIH Lower GI |
| | previous involvement | GI Score by 1 or more | Score by 1 or more points, |
| | | points | except from 0 to 1 |
| Liver | Normal ALT, alkaline | Decrease by 50% | Increase by 2 × ULN |
| | phosphatase, and Total | | |
| | bilirubin after previous | | |
| | elevation of 1 or more | | |
| Lungs | Normal % FEV1 after | Increase by 10% | Decrease by 10% predicted |
| | previous involvement | predicted absolute value | absolute value of % FEV1 |
| | If PFTs not available, | of % FEV1 | If PFTs not available, |
| | NIH Lung Symptom | If PFTs not available, | increase in NIH Lung - |
| | Score 0 after previous | decrease in NIH Lung | Symptom Score by 1 |
| | involvement | Symptom Score by 1 or | or more points, except |
| | | more points | 0 to 1 |
| Joints and | Both NIH Joint and Fascia | Decrease in NIH Joint and | Increase in NIH Joint and Fascia |
| fascia | Score 0 and P-ROM score 25 | Fascia Score by 1 or more | Score by 1 or more points or |
| | after previous involvement | points or increase in P- | decrease in P-ROM score by 1 |
| | by at least | ROM score by 1 point for | point for any site |
| | 1 measure | any site | |
| Global | Clinician overall severity | Clinician overall severity | Clinician overall severity score |
| | score 0 | score decreases by 2 or | increases by 2 or more points on |
| | | more points on a 0-10 scale | a 0-10 scale |

In some embodiments, in cases where cGVHD occurs in subjects that receive a composition of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a complete response to GVHD therapeutic agents.

In some embodiments, in cases where cGVHD occurs in subjects that receive a composition of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a partial response to GVHD therapeutic agents.

### Acute GVHD Incidence

Subjects administered a composition of the disclosure (e.g., a cell population as described herein) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop ≥grade 1 aGVHD, for example, within 30 days post-transplant, 100 days post-transplant, or within another suitable amount of time post-transplant as disclosed herein.

As used herein, an alternate composition lacks one or more cell populations and/or prophylactic agent that are disclosed herein. As examples, an alternate composition lacks one or more of a first population of CD45⁺ cells that comprises, at least, HSPCs, a cell population enriched for Tregs, a third population of CD45⁺ cells that comprises, at least, Tcons, and a prophylactic agent.

Subjects administered a composition of the disclosure (e.g., a cell population as described herein) exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure develop ≥grade 2 aGVHD. In some embodiments, less than about 15% of subjects that are administered a composition of the disclosure develop ≥grade 2 aGVHD. In some embodiments, less than about 10% of subjects that are administered a composition of the disclosure develop ≥grade 2 aGVHD. In some embodiments, less than about 8% of subjects that are administered a composition of the disclosure develop ≥grade 2 aGVHD. In some embodiments, less than about 7% of subjects that are administered a composition of the disclosure develop ≥grade 2 aGVHD.

Subjects administered a composition of the disclosure (e.g., a cell population as described herein) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure develop ≥grade 3 aGVHD. In some embodiments, less than about 15% of subjects that are administered a composition of the disclosure develop ≥grade 3 aGVHD. In some embodiments, less than about 10% of subjects that are administered a composition of the disclosure develop ≥grade 3 aGVHD. In some embodiments, less than about 5% of subjects that are administered a composition of the disclosure develop ≥grade 3 aGVHD. In some embodiments, less than about 3% of subjects that are administered a composition of the disclosure develop ≥grade 3 aGVHD. In some embodiments, less than about 2% of subjects that are administered a composition of the disclosure develop ≥grade 3 aGVHD. In some embodiments, less than about 1% of subjects that are administered a composition of the disclosure develop ≥grade 3 aGVHD.

Subjects administered a composition of the disclosure (e.g., a cell population as described herein) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure develop ≥grade 4 aGVHD. In some embodiments, less than about 5% of subjects that are administered a composition of the disclosure develop ≥grade 4 aGVHD. In some embodiments, less than about 3% of subjects that are administered a composition of the disclosure develop ≥grade 4 aGVHD. In some embodiments, less than about 2% of subjects that are administered a composition of the disclosure develop ≥grade 4 aGVHD. In some embodiments, less than about 1% of subjects that are administered a composition of the disclosure develop ≥grade 4 aGVHD.

The incidence of aGVHD can be assessed after a suitable amount of time elapses post-transplant, for example, about 20 days, about 21 days, about 25 days, about 28 days, about 30 days, about 35 days, about 40 days, about 42 days, about 45 days, about 49 days, about 50 days, about 55 days, about 56 days, about 60 days, about 63 days, about 65 days, about 70 days, about 75 days, about 77 days, about 80 days, about 84 days, about 85 days, about 90 days, about 91 days, about 95 days, about 98 days, about 100 days, about 105 days, about 110 days, about 112 days, about 115 days, about 119 days, about 120 days post-transplant.

The incidence of aGVHD can be calculated based on a population of at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, subjects administered a composition of the disclosure (e.g., a cell population as described herein) have a decreased wherein incidence, severity, timing, or any combination thereof of Grade I to Grade IV acute GVHD (aGVHD), relative to a corresponding human subject that has been administered a standard myeloablative allogeneic hematopoietic stem cell transplant (alloHSCT). In some embodiments, the subject is free of Grade I to Grade IV aGVHD for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

In some embodiments, subjects administered a composition of the disclosure (e.g., a cell population as described herein) have increased aGVHD survival, relative to a corresponding human subject that has been administered a standard myeloablative allogeneic hematopoietic stem cell transplant (alloHSCT). In some embodiments, the subject experiences aGHVD-free survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

In some embodiments, subjects administered a composition of the disclosure (e.g., a cell population as described herein) have decreased incidence of steroid-refractory aGVHD, relative to a corresponding human subject that has been administered a standard myeloablative allogeneic hematopoietic stem cell transplant (alloHSCT). In some embodiments, the subject is free of steroid-refractory aGVHD for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

### No GVHD Prophylaxis

Subjects administered a composition of the disclosure (e.g., a cell population as described herein) in the absence of GVHD prophylactic agents exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) in the absence of GVHD prophylactic agents exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 2 aGVHD, for example, within 30 days post-transplant, 100 days post-transplant, or within another suitable amount of time post-transplant as disclosed herein.

In some embodiments, less than about 50% of subjects that are administered a composition of the disclosure in the absence of GVHD prophylactic agents develop ≥grade 2 aGVHD. In some embodiments, less than about 40% of subjects that are administered a composition of the disclosure in the absence of GVHD prophylactic agents develop ≥grade 2 aGVHD. In some embodiments, less than about 30% of subjects that are administered a composition of the disclosure in the absence of GVHD prophylactic agents develop ≥grade 2 aGVHD. In some embodiments, less than about 25% of subjects that are administered a composition of the disclosure in the absence of GVHD prophylactic agents develop ≥grade 2 aGVHD. In some embodiments, less than about 20% of subjects that are administered a composition of the disclosure in the absence of GVHD prophylactic agents develop ≥grade 2 aGVHD. In some embodiments, less than about 10% of subjects that are administered a composition of the disclosure in the absence of GVHD prophylactic agents develop ≥grade 2 aGVHD.

Subjects administered a composition of the disclosure (e.g., a cell population as described herein) in the absence of GVHD prophylactic agents exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 3 aGVHD, for example, within 30 days post-transplant, 100 days post-transplant, or within another suitable amount of time post-transplant as disclosed herein.

In some embodiments, less than about 40% of subjects that are administered a composition of the disclosure in the absence of GVHD prophylactic agents develop ≥grade 3 aGVHD. In some embodiments, less than about 30% of subjects that are administered a composition of the disclosure in the absence of GVHD prophylactic agents develop ≥grade 3 aGVHD. In some embodiments, less than about 20% of subjects that are administered a composition of the disclosure in the absence of GVHD prophylactic agents develop ≥grade 3 aGVHD. In some embodiments, less than about 10% of subjects that are administered a composition of the disclosure in the absence of GVHD prophylactic agents develop ≥grade 3 aGVHD. In some embodiments, less than about 5% of subjects that are administered a composition of the disclosure in the absence of GVHD prophylactic agents develop ≥grade 3 aGVHD. In some embodiments, less than about 3% of subjects that are administered a composition of the disclosure in the absence of GVHD prophylactic agents develop ≥grade 3 aGVHD. In some embodiments, less than about 2% of subjects that are administered a composition of the disclosure in the absence of GVHD prophylactic agents develop ≥grade 3 aGVHD. In some embodiments, less than about 1% of subjects that are administered a composition of the disclosure in the absence of GVHD prophylactic agents develop ≥grade 3 aGVHD.

Subjects administered a composition of the disclosure (e.g., a cell population as described herein) in the absence of GVHD prophylactic agents exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure in the absence of GVHD prophylactic agents develop ≥grade 4 aGVHD, for example, within 30 days post-transplant, 100 days post-transplant, or within another suitable amount of time post-transplant as disclosed herein.

In some embodiments, less than about 20% of subjects that are administered a composition of the disclosure in the absence of GVHD prophylactic agents develop ≥grade 4 aGVHD. In some embodiments, less than about 15% of subjects that are administered a composition of the disclosure in the absence of GVHD prophylactic agents develop ≥grade 4 aGVHD. In some embodiments, less than about 10% of subjects that are administered a composition of the disclosure in the absence of GVHD prophylactic agents develop ≥grade 4 aGVHD. In some embodiments, less than about 5% of subjects that are administered a composition of the disclosure in the absence of GVHD prophylactic agents develop ≥grade 4 aGVHD. In some embodiments, less than about 3% of subjects that are administered a composition of the disclosure in the absence of GVHD prophylactic agents develop ≥grade 4 aGVHD. In some embodiments, less than about 2% of subjects that are administered a composition of the disclosure in the absence of GVHD prophylactic agents develop ≥grade 4 aGVHD. In some embodiments, less than about 1% of subjects that are administered a composition of the disclosure in the absence of GVHD prophylactic agents develop ≥grade 4 aGVHD.

The absence of GVHD prophylactic agents can refer to cases where no GVHD prophylactic agents are administered to the subject for the first 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 105 days, 110 days, 112 days, 115 days, 119 days, or 120 days post-transplant.

### Single Agent GVHD Prophylaxis

Subjects administered a composition of the disclosure (e.g., a cell population as described herein) and no more than one GVHD prophylactic agent (for example, a single GVHD prophylactic agent) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure and no more than one GVHD prophylactic agent (for example, a single GVHD prophylactic agent) develop ≥grade 2 aGVHD. In some embodiments, less than about 15% of subjects that are administered a composition of the disclosure and no more than one GVHD prophylactic agent (for example, a single GVHD prophylactic agent) develop ≥grade 2 aGVHD. In some embodiments, less than about 10% of subjects that are administered a composition of the disclosure and no more than one GVHD prophylactic agent (for example, a single GVHD prophylactic agent) develop ≥grade 2 aGVHD. In some embodiments, less than about 8% of subjects that are administered a composition of the disclosure and no more than one GVHD prophylactic agent (for example, a single GVHD prophylactic agent) develop ≥grade 2 aGVHD. In some embodiments, less than about 7% of subjects that are administered a composition of the disclosure and no more than one GVHD prophylactic agent (for example, a single GVHD prophylactic agent) develop ≥grade 2 aGVHD.

Subjects administered a composition of the disclosure (e.g., a cell population as described herein) and no more than one GVHD prophylactic agent (for example, a single GVHD prophylactic agent) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure and no more than one GVHD prophylactic agent (for example, a single GVHD prophylactic agent) develop ≥grade 3 aGVHD. In some embodiments, less than about 15% of subjects that are administered a composition of the disclosure and no more than one GVHD prophylactic agent (for example, a single GVHD prophylactic agent) develop ≥grade 3 aGVHD. In some embodiments, less than about 10% of subjects that are administered a composition of the disclosure and no more than one GVHD prophylactic agent (for example, a single GVHD prophylactic agent) develop ≥grade 3 aGVHD. In some embodiments, less than about 5% of subjects that are administered a composition of the disclosure and no more than one GVHD prophylactic agent (for example, a single GVHD prophylactic agent) develop ≥grade 3 aGVHD. In some embodiments, less than about 3% of subjects that are administered a composition of the disclosure and no more than one GVHD prophylactic agent (for example, a single GVHD prophylactic agent) develop ≥grade 3 aGVHD. In some embodiments, less than about 2% of subjects that are administered a composition of the disclosure and no more than one GVHD prophylactic agent (for example, a single GVHD prophylactic agent) develop ≥grade 3 aGVHD. In some embodiments, less than about 1% of subjects that are administered a composition of the disclosure and no more than one GVHD prophylactic agent (for example, a single GVHD prophylactic agent) develop ≥grade 3 aGVHD.

Subjects administered a composition of the disclosure (e.g., a cell population as described herein) and no more than one GVHD prophylactic agent (for example, a single GVHD prophylactic agent) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and no more than one GVHD prophylactic agent (for example, a single GVHD prophylactic agent) develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure and no more than one GVHD prophylactic agent (for example, a single GVHD prophylactic agent) develop ≥grade 4 aGVHD. In some embodiments, less than about 5% of subjects that are administered a composition of the disclosure and no more than one GVHD prophylactic agent (for example, a single GVHD prophylactic agent) develop ≥grade 4 aGVHD. In some embodiments, less than about 3% of subjects that are administered a composition of the disclosure and no more than one GVHD prophylactic agent (for example, a single GVHD prophylactic agent) develop ≥grade 4 aGVHD. In some embodiments, less than about 2% of subjects that are administered a composition of the disclosure and no more than one GVHD prophylactic agent (for example, a single GVHD prophylactic agent) develop ≥grade 4 aGVHD. In some embodiments, less than about 1% of subjects that are administered a composition of the disclosure and no more than one GVHD prophylactic agent (for example, a single GVHD prophylactic agent) develop ≥grade 4 aGVHD.

A single GVHD prophylactic agent can be tacrolimus.

A single GVHD prophylactic agent can be sirolimus.

The no more than one GVHD prophylactic agent (for example, a single GVHD prophylactic agent) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the no more than one GVHD prophylactic agent (for example, a single GVHD prophylactic agent) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Low Dose GVHD Prophylaxis

Subjects administered a composition of the disclosure (e.g., a cell population as described herein) and a low dose of a GVHD prophylactic agent (for example, a single GVHD prophylactic agent at a low dose) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop ≥grade 1 aGVHD, for example, within 30 days post-transplant, 100 days post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure and a low dose of a GVHD prophylactic agent (for example, a single GVHD prophylactic agent at a low dose) develop ≥grade 2 aGVHD. In some embodiments, less than about 15% of subjects that are administered a composition of the disclosure and a low dose of a GVHD prophylactic agent (for example, a single GVHD prophylactic agent at a low dose) develop ≥grade 2 aGVHD. In some embodiments, less than about 10% of subjects that are administered a composition of the disclosure and a low dose of a GVHD prophylactic agent (for example, a single GVHD prophylactic agent at a low dose) develop ≥grade 2 aGVHD. In some embodiments, less than about 8% of subjects that are administered a composition of the disclosure and a low dose of a GVHD prophylactic agent (for example, a single GVHD prophylactic agent at a low dose) develop ≥grade 2 aGVHD. In some embodiments, less than about 7% of subjects that are administered a composition of the disclosure and a low dose of a GVHD prophylactic agent (for example, a single GVHD prophylactic agent at a low dose) develop ≥grade 2 aGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure and a low dose of a GVHD prophylactic agent (for example, a single GVHD prophylactic agent at a low dose) develop ≥grade 3 aGVHD. In some embodiments, less than about 15% of subjects that are administered a composition of the disclosure and a low dose of a GVHD prophylactic agent (for example, a single GVHD prophylactic agent at a low dose) develop ≥grade 3 aGVHD. In some embodiments, less than about 10% of subjects that are administered a composition of the disclosure and a low dose of a GVHD prophylactic agent (for example, a single GVHD prophylactic agent at a low dose) develop ≥grade 3 aGVHD. In some embodiments, less than about 5% of subjects that are administered a composition of the disclosure and a low dose of a GVHD prophylactic agent (for example, a single GVHD prophylactic agent at a low dose) develop ≥grade 3 aGVHD. In some embodiments, less than about 3% of subjects that are administered a composition of the disclosure and a low dose of a GVHD prophylactic agent (for example, a single GVHD prophylactic agent at a low dose) develop ≥grade 3 aGVHD. In some embodiments, less than about 2% of subjects that are administered a composition of the disclosure and a low dose of a GVHD prophylactic agent (for example, a single GVHD prophylactic agent at a low dose) develop ≥grade 3 aGVHD. In some embodiments, less than about 1% of subjects that are administered a composition of the disclosure and a low dose of a GVHD prophylactic agent (for example, a single GVHD prophylactic agent at a low dose) develop ≥grade 3 aGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and a low dose of a GVHD prophylactic agent (for example, a single GVHD prophylactic agent at a low dose) develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure and a low dose of a GVHD prophylactic agent (for example, a single GVHD prophylactic agent at a low dose) develop ≥grade 4 aGVHD. In some embodiments, less than about 5% of subjects that are administered a composition of the disclosure and a low dose of a GVHD prophylactic agent (for example, a single GVHD prophylactic agent at a low dose) develop ≥grade 4 aGVHD. In some embodiments, less than about 3% of subjects that are administered a composition of the disclosure and a low dose of a GVHD prophylactic agent (for example, a single GVHD prophylactic agent at a low dose) develop ≥grade 4 aGVHD. In some embodiments, less than about 2% of subjects that are administered a composition of the disclosure and a low dose of a GVHD prophylactic agent (for example, a single GVHD prophylactic agent at a low dose) develop ≥grade 4 aGVHD. In some embodiments, less than about 1% of subjects that are administered a composition of the disclosure and a low dose of a GVHD prophylactic agent (for example, a single GVHD prophylactic agent at a low dose) develop ≥grade 4 aGVHD.

A low dose of a GVHD prophylactic agent can be, for example a target trough level of less than about 25 ng/mL, less than about 20 ng/mL, less than about 15 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a low dose of a GVHD prophylactic is a target trough level of approximately 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a low dose of a GVHD prophylactic agent is tacrolimus with a target trough level of approximately 5 ng/mL to approximately 10 ng/mL. In some embodiments, a low dose of a GVHD prophylactic agent is tacrolimus with a target trough level of approximately 4 ng/mL to approximately 6 ng/mL.

In some embodiments, a low dose of a GVHD prophylactic agent is sirolimus with a target trough level of approximately 3 ng/mL to approximately 8 ng/mL. In some embodiments, a low dose of a GVHD prophylactic agent is sirolimus with a target trough level of approximately 4 ng/mL to approximately 8 ng/mL.

The low dose GVHD prophylactic agent (for example, single GVHD prophylactic agent at a low dose) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the low dose GVHD prophylactic agent (for example, single GVHD prophylactic agent at a low dose) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Chronic GVHD Incidence

Subjects administered a composition of the disclosure (e.g., a cell population as described herein) exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure develop ≥grade 2 cGVHD. In some embodiments, less than about 40% of subjects that are administered a composition of the disclosure develop ≥grade 2 cGVHD. In some embodiments, less than about 30% of subjects that are administered a composition of the disclosure develop ≥grade 2 cGVHD. In some embodiments, less than about 20% of subjects that are administered a composition of the disclosure develop ≥grade 2 cGVHD. In some embodiments, less than about 10% of subjects that are administered a composition of the disclosure develop ≥grade 2 cGVHD. In some embodiments, less than about 5% of subjects that are administered a composition of the disclosure develop ≥grade 2 cGVHD.

Subjects administered a composition of the disclosure (e.g., a cell population as described herein) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure develop ≥grade 3 cGVHD. In some embodiments, less than about 40% of subjects that are administered a composition of the disclosure develop ≥grade 3 cGVHD. In some embodiments, less than about 30% of subjects that are administered a composition of the disclosure develop ≥grade 3 cGVHD. In some embodiments, less than about 20% of subjects that are administered a composition of the disclosure develop ≥grade 3 cGVHD. In some embodiments, less than about 15% of subjects that are administered a composition of the disclosure develop ≥grade 3 cGVHD. In some embodiments, less than about 10% of subjects that are administered a composition of the disclosure develop ≥grade 3 cGVHD. In some embodiments, less than about 5% of subjects that are administered a composition of the disclosure develop ≥grade 3 cGVHD. In some embodiments, less than about 3% of subjects that are administered a composition of the disclosure develop ≥grade 3 cGVHD. In some embodiments, less than about 2% of subjects that are administered a composition of the disclosure develop ≥grade 3 cGVHD. In some embodiments, less than about 1% of subjects that are administered a composition of the disclosure develop ≥grade 3 cGVHD.

Subjects administered a composition of the disclosure (e.g., a cell population as described herein) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure develop ≥grade 4 cGVHD. In some embodiments, less than about 30% of subjects that are administered a composition of the disclosure develop ≥grade 4 cGVHD. In some embodiments, less than about 20% of subjects that are administered a composition of the disclosure develop ≥grade 4 cGVHD. In some embodiments, less than about 10% of subjects that are administered a composition of the disclosure develop ≥grade 4 cGVHD. In some embodiments, less than about 5% of subjects that are administered a composition of the disclosure develop ≥grade 4 cGVHD. In some embodiments, less than about 3% of subjects that are administered a composition of the disclosure develop ≥grade 4 cGVHD. In some embodiments, less than about 2% of subjects that are administered a composition of the disclosure develop ≥grade 4 cGVHD. In some embodiments, less than about 1% of subjects that are administered a composition of the disclosure develop ≥grade 4 cGVHD.

Subjects administered a composition of the disclosure (e.g., a cell population as described herein) exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop mild to severe cGVHD, for example, within 365 days post-transplant, two years post-transplant, or within another suitable amount of time post-transplant as disclosed herein.

Subjects administered a composition of the disclosure (e.g., a cell population as described herein) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure develop moderate to severe cGVHD. In some embodiments, less than about 40% of subjects that are administered a composition of the disclosure develop moderate to severe cGVHD. In some embodiments, less than about 30% of subjects that are administered a composition of the disclosure develop moderate to severe cGVHD. In some embodiments, less than about 20% of subjects that are administered a composition of the disclosure develop moderate to severe cGVHD. In some embodiments, less than about 10% of subjects that are administered a composition of the disclosure develop moderate to severe cGVHD. In some embodiments, less than about 5% of subjects that are administered a composition of the disclosure develop moderate to severe cGVHD.

Subjects administered a composition of the disclosure (e.g., a cell population as described herein) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop severe cGVHD, for example, within 365 days post-transplant, two years post-transplant, or within another suitable amount of time post-transplant as disclosed herein.

In some embodiments, less than about 50% of subjects that are administered a composition of the disclosure develop severe cGVHD. In some embodiments, less than about 40% of subjects that are administered a composition of the disclosure develop severe cGVHD. In some embodiments, less than about 30% of subjects that are administered a composition of the disclosure develop severe cGVHD. In some embodiments, less than about 20% of subjects that are administered a composition of the disclosure develop severe cGVHD. In some embodiments, less than about 15% of subjects that are administered a composition of the disclosure develop severe cGVHD. In some embodiments, less than about 10% of subjects that are administered a composition of the disclosure develop severe cGVHD. In some embodiments, less than about 5% of subjects that are administered a composition of the disclosure develop severe cGVHD. In some embodiments, less than about 3% of subjects that are administered a composition of the disclosure develop severe cGVHD. In some embodiments, less than about 2% of subjects that are administered a composition of the disclosure develop severe cGVHD. In some embodiments, less than about 1% of subjects that are administered a composition of the disclosure develop severe cGVHD.

The incidence of cGVHD can be assessed after a suitable amount of time elapses post-transplant, for example, about 150 days, about 200 days, about 365 days, about 1.5 years, about 2 years, about 2.5 years, about 3 years, about 3.5 years, about 4 years, about 4.5 years, or about 5 years post-transplant.

The incidence of cGVHD can be calculated based on a population of at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, subjects administered a composition of the disclosure (e.g., a cell population as described herein) have a decreased wherein incidence, severity, timing, or any combination thereof of Grade I to Grade IV chronic GVHD (cGVHD) or severe cGVHD, relative to a corresponding human subject that has been administered a standard myeloablative allogeneic hematopoietic stem cell transplant (alloHSCT). In some embodiments, the subject is free of Grade I to Grade IV cGVHD or severe cGVHD for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

In some embodiments, subjects administered a composition of the disclosure (e.g., a cell population as described herein) have increased cGVHD survival, relative to a corresponding human subject that has been administered a standard myeloablative allogeneic hematopoietic stem cell transplant (alloHSCT). In some embodiments, the subject experiences cGHVD-free survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.10 years or more, approximately 1.15 years or more, approximately 1.20 years or more, approximately 1.25 years or more, approximately 1.30 years or more, approximately 1.35 years or more, approximately 1.40 years or more, approximately 1.45 years or more, approximately 1.50 years or more, approximately 1.55 years or more, approximately 1.60 years or more, approximately 1.65 years or more, approximately 1.70 years or more, approximately 1.75 years or more, approximately 1.80 years or more, approximately 1.85 years or more, approximately 1.90 years or more, approximately 1.95 years or more, approximately 2 years or more, approximately 2.10 years or more, approximately 2.15 years or more, approximately 2.20 years or more, approximately 2.25 years or more, approximately 2.30 years or more, approximately 2.35 years or more, approximately 2.40 years or more, approximately 2.45 years or more, approximately 2.50 years or more, approximately 2.55 years or more, approximately 2.60 years or more, approximately 2.65 years or more, approximately 2.70 years or more, approximately 2.75 years or more, approximately 2.80 years or more, approximately 2.85 years or more, approximately 2.90 years or more, approximately 2.95 years or more, approximately 3 years or more, approximately 3.10 years or more, approximately 3.15 years or more, approximately 3.20 years or more, approximately 3.25 years or more, approximately 3.30 years or more, approximately 3.35 years or more, approximately 3.40 years or more, approximately 3.45 years or more, approximately 3.50 years or more, approximately 3.55 years or more, approximately 3.60 years or more, approximately 3.65 years or more, approximately 3.70 years or more, approximately 3.75 years or more, approximately 3.80 years or more, approximately 3.85 years or more, approximately 3.90 years or more, approximately 3.95 years or more, approximately 4.10 years or more, approximately 4.15 years or more, approximately 4.20 years or more, approximately 4.25 years or more, approximately 4.30 years or more, approximately 4.35 years or more, approximately 4.40 years or more, approximately 4.45 years or more, approximately 4.50 years or more, approximately 4.55 years or more, approximately 4.60 years or more, approximately 4.65 years or more, approximately 4.70 years or more, approximately 4.75 years or more, approximately 4.80 years or more, approximately 4.85 years or more, approximately 4.90 years or more, approximately 4.95 years or more, or approximately 5 years or more after administration of the composition of the disclosure.

### No GVHD Prophylaxis

Subjects administered a composition of the disclosure (e.g., a cell population as described herein) in the absence of GVHD prophylactic agents exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) in the absence of GVHD prophylactic agents exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop ≥grade 2 cGVHD. In some embodiments, less than about 40% of subjects that are administered a composition of the disclosure in the absence of GVHD prophylactic agents develop ≥grade 2 cGVHD. In some embodiments, less than about 30% of subjects that are administered a composition of the disclosure in the absence of GVHD prophylactic agents develop ≥grade 2 cGVHD. In some embodiments, less than about 25% of subjects that are administered a composition of the disclosure in the absence of GVHD prophylactic agents develop ≥grade 2 cGVHD. In some embodiments, less than about 20% of subjects that are administered a composition of the disclosure in the absence of GVHD prophylactic agents develop ≥grade 2 cGVHD. In some embodiments, less than about 10% of subjects that are administered a composition of the disclosure in the absence of GVHD prophylactic agents develop ≥grade 2 cGVHD. In some embodiments, less than about 5% of subjects that are administered a composition of the disclosure in the absence of GVHD prophylactic agents develop ≥grade 2 cGVHD.

Subjects administered a composition of the disclosure (e.g., a cell population as described herein) in the absence of GVHD prophylactic agents exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop ≥grade 3 cGVHD. In some embodiments, less than about 40% of subjects that are administered a composition of the disclosure in the absence of GVHD prophylactic agents develop ≥grade 3 cGVHD. In some embodiments, less than about 30% of subjects that are administered a composition of the disclosure in the absence of GVHD prophylactic agents develop ≥grade 3 cGVHD. In some embodiments, less than about 20% of subjects that are administered a composition of the disclosure in the absence of GVHD prophylactic agents develop ≥grade 3 cGVHD. In some embodiments, less than about 10% of subjects that are administered a composition of the disclosure in the absence of GVHD prophylactic agents develop ≥grade 3 cGVHD. In some embodiments, less than about 5% of subjects that are administered a composition of the disclosure in the absence of GVHD prophylactic agents develop ≥grade 3 cGVHD. In some embodiments, less than about 3% of subjects that are administered a composition of the disclosure in the absence of GVHD prophylactic agents develop ≥grade 3 cGVHD. In some embodiments, less than about 2% of subjects that are administered a composition of the disclosure in the absence of GVHD prophylactic agents develop ≥grade 3 cGVHD. In some embodiments, less than about 1% of subjects that are administered a composition of the disclosure in the absence of GVHD prophylactic agents develop ≥grade 3 cGVHD.

Subjects administered a composition of the disclosure (e.g., a cell population as described herein) in the absence of GVHD prophylactic agents exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 40%, or less than about 50% of subjects that are administered a composition of the disclosure in the absence of GVHD prophylactic agents develop ≥grade 4 cGVHD, for example, within 365 days post-transplant, two years post-transplant, or within another suitable amount of time post-transplant as disclosed herein.

In some embodiments, less than about 30% of subjects that are administered a composition of the disclosure in the absence of GVHD prophylactic agents develop ≥grade 4 cGVHD. In some embodiments, less than about 20% of subjects that are administered a composition of the disclosure in the absence of GVHD prophylactic agents develop ≥grade 4 cGVHD. In some embodiments, less than about 15% of subjects that are administered a composition of the disclosure in the absence of GVHD prophylactic agents develop ≥grade 4 cGVHD. In some embodiments, less than about 10% of subjects that are administered a composition of the disclosure in the absence of GVHD prophylactic agents develop ≥grade 4 cGVHD. In some embodiments, less than about 5% of subjects that are administered a composition of the disclosure in the absence of GVHD prophylactic agents develop ≥grade 4 cGVHD. In some embodiments, less than about 3% of subjects that are administered a composition of the disclosure in the absence of GVHD prophylactic agents develop ≥grade 4 cGVHD. In some embodiments, less than about 2% of subjects that are administered a composition of the disclosure in the absence of GVHD prophylactic agents develop ≥grade 4 cGVHD. In some embodiments, less than about 1% of subjects that are administered a composition of the disclosure in the absence of GVHD prophylactic agents develop ≥grade 4 cGVHD.

Subjects administered a composition of the disclosure (e.g., a cell population as described herein) in the absence of GVHD prophylactic agents exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop mild to severe cGVHD, for example, within 365 days post-transplant, two years post-transplant, or within another suitable amount of time post-transplant as disclosed herein.

Subjects administered a composition of the disclosure (e.g., a cell population as described herein) in the absence of GVHD prophylactic agents exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop moderate to severe cGVHD. In some embodiments, less than about 40% of subjects that are administered a composition of the disclosure in the absence of GVHD prophylactic agents develop moderate to severe cGVHD. In some embodiments, less than about 30% of subjects that are administered a composition of the disclosure in the absence of GVHD prophylactic agents develop moderate to severe cGVHD. In some embodiments, less than about 25% of subjects that are administered a composition of the disclosure in the absence of GVHD prophylactic agents develop moderate to severe cGVHD. In some embodiments, less than about 20% of subjects that are administered a composition of the disclosure in the absence of GVHD prophylactic agents develop moderate to severe cGVHD. In some embodiments, less than about 10% of subjects that are administered a composition of the disclosure in the absence of GVHD prophylactic agents develop moderate to severe cGVHD. In some embodiments, less than about 5% of subjects that are administered a composition of the disclosure in the absence of GVHD prophylactic agents develop moderate to severe cGVHD.

Subjects administered a composition of the disclosure (e.g., a cell population as described herein) in the absence of GVHD prophylactic agents exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop severe cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop severe cGVHD. In some embodiments, less than about 40% of subjects that are administered a composition of the disclosure in the absence of GVHD prophylactic agents develop severe cGVHD. In some embodiments, less than about 30% of subjects that are administered a composition of the disclosure in the absence of GVHD prophylactic agents develop severe cGVHD. In some embodiments, less than about 20% of subjects that are administered a composition of the disclosure in the absence of GVHD prophylactic agents develop severe cGVHD. In some embodiments, less than about 10% of subjects that are administered a composition of the disclosure in the absence of GVHD prophylactic agents develop severe cGVHD. In some embodiments, less than about 5% of subjects that are administered a composition of the disclosure in the absence of GVHD prophylactic agents develop severe cGVHD. In some embodiments, less than about 3% of subjects that are administered a composition of the disclosure in the absence of GVHD prophylactic agents develop severe cGVHD. In some embodiments, less than about 2% of subjects that are administered a composition of the disclosure in the absence of GVHD prophylactic agents develop severe cGVHD. In some embodiments, less than about 1% of subjects that are administered a composition of the disclosure in the absence of GVHD prophylactic agents develop severe cGVHD.

The absence of GVHD prophylactic agents can refer to cases where no GVHD prophylactic agents are administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant.

### Single Agent GVHD Prophylaxis

Subjects administered a composition of the disclosure (e.g., a cell population as described herein) and no more than one GVHD prophylactic agent (for example, a single GVHD prophylactic agent) exhibit a low incidence of ≥grade 1 cGVHD, for example, a lower incidence of ≥grade 1 cGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop ≥grade 2 cGVHD. In some embodiments, less than about 40% of subjects that are administered a composition of the disclosure and no more than one GVHD prophylactic agent (for example, a single GVHD prophylactic agent) develop ≥grade 2 cGVHD. In some embodiments, less than about 30% of subjects that are administered a composition of the disclosure and no more than one GVHD prophylactic agent (for example, a single GVHD prophylactic agent) develop ≥grade 2 cGVHD. In some embodiments, less than about 20% of subjects that are administered a composition of the disclosure and no more than one GVHD prophylactic agent (for example, a single GVHD prophylactic agent) develop ≥grade 2 cGVHD. In some embodiments, less than about 10% of subjects that are administered a composition of the disclosure and no more than one GVHD prophylactic agent (for example, a single GVHD prophylactic agent) develop ≥grade 2 cGVHD. In some embodiments, less than about 5% of subjects that are administered a composition of the disclosure and no more than one GVHD prophylactic agent (for example, a single GVHD prophylactic agent) develop ≥grade 2 cGVHD.

Subjects administered a composition of the disclosure (e.g., a cell population as described herein) and no more than one GVHD prophylactic agent (for example, a single GVHD prophylactic agent) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop ≥grade 3 cGVHD. In some embodiments, less than about 40% of subjects that are administered a composition of the disclosure and no more than one GVHD prophylactic agent (for example, a single GVHD prophylactic agent) develop ≥grade 3 cGVHD. In some embodiments, less than about 30% of subjects that are administered a composition of the disclosure and no more than one GVHD prophylactic agent (for example, a single GVHD prophylactic agent) develop ≥grade 3 cGVHD. In some embodiments, less than about 20% of subjects that are administered a composition of the disclosure and no more than one GVHD prophylactic agent (for example, a single GVHD prophylactic agent) develop ≥grade 3 cGVHD. In some embodiments, less than about 15% of subjects that are administered a composition of the disclosure and no more than one GVHD prophylactic agent (for example, a single GVHD prophylactic agent) develop ≥grade 3 cGVHD. In some embodiments, less than about 10% of subjects that are administered a composition of the disclosure and no more than one GVHD prophylactic agent (for example, a single GVHD prophylactic agent) develop ≥grade 3 cGVHD. In some embodiments, less than about 5% of subjects that are administered a composition of the disclosure and no more than one GVHD prophylactic agent (for example, a single GVHD prophylactic agent) develop ≥grade 3 cGVHD. In some embodiments, less than about 3% of subjects that are administered a composition of the disclosure and no more than one GVHD prophylactic agent (for example, a single GVHD prophylactic agent) develop ≥grade 3 cGVHD. In some embodiments, less than about 2% of subjects that are administered a composition of the disclosure and no more than one GVHD prophylactic agent (for example, a single GVHD prophylactic agent) develop ≥grade 3 cGVHD. In some embodiments, less than about 1% of subjects that are administered a composition of the disclosure and no more than one GVHD prophylactic agent (for example, a single GVHD prophylactic agent) develop ≥grade 3 cGVHD.

Subjects administered a composition of the disclosure (e.g., a cell population as described herein) and no more than one GVHD prophylactic agent (for example, a single GVHD prophylactic agent) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and no more than one GVHD prophylactic agent (for example, a single GVHD prophylactic agent) develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure and no more than one GVHD prophylactic agent (for example, a single GVHD prophylactic agent) develop ≥grade 4 cGVHD. In some embodiments, less than about 30% of subjects that are administered a composition of the disclosure and no more than one GVHD prophylactic agent (for example, a single GVHD prophylactic agent) develop ≥grade 4 cGVHD. In some embodiments, less than about 20% of subjects that are administered a composition of the disclosure and no more than one GVHD prophylactic agent (for example, a single GVHD prophylactic agent) develop ≥grade 4 cGVHD. In some embodiments, less than about 10% of subjects that are administered a composition of the disclosure and no more than one GVHD prophylactic agent (for example, a single GVHD prophylactic agent) develop ≥grade 4 cGVHD. In some embodiments, less than about 5% of subjects that are administered a composition of the disclosure and no more than one GVHD prophylactic agent (for example, a single GVHD prophylactic agent) develop ≥grade 4 cGVHD. In some embodiments, less than about 3% of subjects that are administered a composition of the disclosure and no more than one GVHD prophylactic agent (for example, a single GVHD prophylactic agent) develop ≥grade 4 cGVHD. In some embodiments, less than about 2% of subjects that are administered a composition of the disclosure and no more than one GVHD prophylactic agent (for example, a single GVHD prophylactic agent) develop ≥grade 4 cGVHD. In some embodiments, less than about 1% of subjects that are administered a composition of the disclosure and no more than one GVHD prophylactic agent (for example, a single GVHD prophylactic agent) develop ≥grade 4 cGVHD.

Subjects administered a composition of the disclosure (e.g., a cell population as described herein) and no more than one GVHD prophylactic agent (for example, a single GVHD prophylactic agent) exhibit a low incidence of mild to severe cGVHD, for example, a lower incidence of mild to severe cGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop mild to severe cGVHD, for example, within 365 days post-transplant, two years post-transplant, or within another suitable amount of time post-transplant as disclosed herein.

Subjects administered a composition of the disclosure (e.g., a cell population as described herein) and no more than one GVHD prophylactic agent (for example, a single GVHD prophylactic agent) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop moderate to severe cGVHD. In some embodiments, less than about 40% of subjects that are administered a composition of the disclosure and no more than one GVHD prophylactic agent (for example, a single GVHD prophylactic agent) develop moderate to severe cGVHD. In some embodiments, less than about 30% of subjects that are administered a composition of the disclosure and no more than one GVHD prophylactic agent (for example, a single GVHD prophylactic agent) develop moderate to severe cGVHD. In some embodiments, less than about 20% of subjects that are administered a composition of the disclosure and no more than one GVHD prophylactic agent (for example, a single GVHD prophylactic agent) develop moderate to severe cGVHD. In some embodiments, less than about 10% of subjects that are administered a composition of the disclosure and no more than one GVHD prophylactic agent (for example, a single GVHD prophylactic agent) develop moderate to severe cGVHD. In some embodiments, less than about 5% of subjects that are administered a composition of the disclosure and no more than one GVHD prophylactic agent (for example, a single GVHD prophylactic agent) develop moderate to severe cGVHD.

Subjects administered a composition of the disclosure (e.g., a cell population as described herein) and no more than one GVHD prophylactic agent (for example, a single GVHD prophylactic agent) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop severe cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop severe cGVHD. In some embodiments, less than about 40% of subjects that are administered a composition of the disclosure and no more than one GVHD prophylactic agent (for example, a single GVHD prophylactic agent) develop severe cGVHD. In some embodiments, less than about 30% of subjects that are administered a composition of the disclosure and no more than one GVHD prophylactic agent (for example, a single GVHD prophylactic agent) develop severe cGVHD. In some embodiments, less than about 20% of subjects that are administered a composition of the disclosure and no more than one GVHD prophylactic agent (for example, a single GVHD prophylactic agent) develop severe cGVHD. In some embodiments, less than about 15% of subjects that are administered a composition of the disclosure and no more than one GVHD prophylactic agent (for example, a single GVHD prophylactic agent) develop severe cGVHD. In some embodiments, less than about 10% of subjects that are administered a composition of the disclosure and no more than one GVHD prophylactic agent (for example, a single GVHD prophylactic agent) develop severe cGVHD. In some embodiments, less than about 5% of subjects that are administered a composition of the disclosure and no more than one GVHD prophylactic agent (for example, a single GVHD prophylactic agent) develop severe cGVHD. In some embodiments, less than about 3% of subjects that are administered a composition of the disclosure and no more than one GVHD prophylactic agent (for example, a single GVHD prophylactic agent) develop severe cGVHD. In some embodiments, less than about 2% of subjects that are administered a composition of the disclosure and no more than one GVHD prophylactic agent (for example, a single GVHD prophylactic agent) develop severe cGVHD. In some embodiments, less than about 1% of subjects that are administered a composition of the disclosure and no more than one GVHD prophylactic agent (for example, a single GVHD prophylactic agent) develop severe cGVHD.

A single GVHD prophylactic agent can be tacrolimus.

A single GVHD prophylactic agent can be sirolimus.

The no more than one GVHD prophylactic agent (for example, a single GVHD prophylactic agent) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the no more than one GVHD prophylactic agent (for example, a single GVHD prophylactic agent) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Low Dose GVHD Prophylaxis

Subjects administered a composition of the disclosure (e.g., a cell population as described herein) and a low dose of a GVHD prophylactic agent (for example, a single GVHD prophylactic agent at a low dose) exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop ≥grade 1 cGVHD, for example, within 365 days post-transplant, two years post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop ≥grade 2 cGVHD. In some embodiments, less than about 40% of subjects that are administered a composition of the disclosure and a low dose of a GVHD prophylactic agent (for example, a single GVHD prophylactic agent at a low dose) develop ≥grade 2 cGVHD. In some embodiments, less than about 30% of subjects that are administered a composition of the disclosure and a low dose of a GVHD prophylactic agent (for example, a single GVHD prophylactic agent at a low dose) develop ≥grade 2 cGVHD. In some embodiments, less than about 20% of subjects that are administered a composition of the disclosure and a low dose of a GVHD prophylactic agent (for example, a single GVHD prophylactic agent at a low dose) develop ≥grade 2 cGVHD. In some embodiments, less than about 15% of subjects that are administered a composition of the disclosure and a low dose of a GVHD prophylactic agent (for example, a single GVHD prophylactic agent at a low dose) develop ≥grade 2 cGVHD. In some embodiments, less than about 10% of subjects that are administered a composition of the disclosure and a low dose of a GVHD prophylactic agent (for example, a single GVHD prophylactic agent at a low dose) develop ≥grade 2 cGVHD. In some embodiments, less than about 5% of subjects that are administered a composition of the disclosure and a low dose of a GVHD prophylactic agent (for example, a single GVHD prophylactic agent at a low dose) develop ≥grade 2 cGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop ≥grade 3 cGVHD. In some embodiments, less than about 40% of subjects that are administered a composition of the disclosure and a low dose of a GVHD prophylactic agent (for example, a single GVHD prophylactic agent at a low dose) develop ≥grade 3 cGVHD. In some embodiments, less than about 30% of subjects that are administered a composition of the disclosure and a low dose of a GVHD prophylactic agent (for example, a single GVHD prophylactic agent at a low dose) develop ≥grade 3 cGVHD. In some embodiments, less than about 20% of subjects that are administered a composition of the disclosure and a low dose of a GVHD prophylactic agent (for example, a single GVHD prophylactic agent at a low dose) develop ≥grade 3 cGVHD. In some embodiments, less than about 15% of subjects that are administered a composition of the disclosure and a low dose of a GVHD prophylactic agent (for example, a single GVHD prophylactic agent at a low dose) develop ≥grade 3 cGVHD. In some embodiments, less than about 10% of subjects that are administered a composition of the disclosure and a low dose of a GVHD prophylactic agent (for example, a single GVHD prophylactic agent at a low dose) develop ≥grade 3 cGVHD. In some embodiments, less than about 5% of subjects that are administered a composition of the disclosure and a low dose of a GVHD prophylactic agent (for example, a single GVHD prophylactic agent at a low dose) develop ≥grade 3 cGVHD. In some embodiments, less than about 3% of subjects that are administered a composition of the disclosure and a low dose of a GVHD prophylactic agent (for example, a single GVHD prophylactic agent at a low dose) develop ≥grade 3 cGVHD. In some embodiments, less than about 2% of subjects that are administered a composition of the disclosure and a low dose of a GVHD prophylactic agent (for example, a single GVHD prophylactic agent at a low dose) develop ≥grade 3 cGVHD. In some embodiments, less than about 1% of subjects that are administered a composition of the disclosure and a low dose of a GVHD prophylactic agent (for example, a single GVHD prophylactic agent at a low dose) develop ≥grade 3 cGVHD.

Subjects administered a composition of the disclosure (e.g., a cell population as described herein) and a low dose of a GVHD prophylactic agent (for example, a single GVHD prophylactic agent at a low dose) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and a low dose of a GVHD prophylactic agent (for example, a single GVHD prophylactic agent at a low dose) develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure and a low dose of a GVHD prophylactic agent (for example, a single GVHD prophylactic agent at a low dose) develop ≥grade 4 cGVHD. In some embodiments, less than about 30% of subjects that are administered a composition of the disclosure and a low dose of a GVHD prophylactic agent (for example, a single GVHD prophylactic agent at a low dose) develop ≥grade 4 cGVHD. In some embodiments, less than about 20% of subjects that are administered a composition of the disclosure and a low dose of a GVHD prophylactic agent (for example, a single GVHD prophylactic agent at a low dose) develop ≥grade 4 cGVHD. In some embodiments, less than about 10% of subjects that are administered a composition of the disclosure and a low dose of a GVHD prophylactic agent (for example, a single GVHD prophylactic agent at a low dose) develop ≥grade 4 cGVHD. In some embodiments, less than about 5% of subjects that are administered a composition of the disclosure and a low dose of a GVHD prophylactic agent (for example, a single GVHD prophylactic agent at a low dose) develop ≥grade 4 cGVHD. In some embodiments, less than about 3% of subjects that are administered a composition of the disclosure and a low dose of a GVHD prophylactic agent (for example, a single GVHD prophylactic agent at a low dose) develop ≥grade 4 cGVHD. In some embodiments, less than about 2% of subjects that are administered a composition of the disclosure and a low dose of a GVHD prophylactic agent (for example, a single GVHD prophylactic agent at a low dose) develop ≥grade 4 cGVHD. In some embodiments, less than about 1% of subjects that are administered a composition of the disclosure and a low dose of a GVHD prophylactic agent (for example, a single GVHD prophylactic agent at a low dose) develop ≥grade 4 cGVHD.

Subjects administered a composition of the disclosure (e.g., a cell population as described herein) and a low dose of a GVHD prophylactic agent (for example, a single GVHD prophylactic agent at a low dose) exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop mild to severe cGVHD, for example, within 365 days post-transplant, two years post-transplant, or within another suitable amount of time post-transplant as disclosed herein.

Subjects administered a composition of the disclosure (e.g., a cell population as described herein) and a low dose of a GVHD prophylactic agent (for example, a single GVHD prophylactic agent at a low dose) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop moderate to severe cGVHD. In some embodiments, less than about 40% of subjects that are administered a composition of the disclosure and a low dose of a GVHD prophylactic agent (for example, a single GVHD prophylactic agent at a low dose) develop moderate to severe cGVHD. In some embodiments, less than about 30% of subjects that are administered a composition of the disclosure and a low dose of a GVHD prophylactic agent (for example, a single GVHD prophylactic agent at a low dose) develop moderate to severe cGVHD. In some embodiments, less than about 20% of subjects that are administered a composition of the disclosure and a low dose of a GVHD prophylactic agent (for example, a single GVHD prophylactic agent at a low dose) develop moderate to severe cGVHD. In some embodiments, less than about 15% of subjects that are administered a composition of the disclosure and a low dose of a GVHD prophylactic agent (for example, a single GVHD prophylactic agent at a low dose) develop moderate to severe cGVHD. In some embodiments, less than about 10% of subjects that are administered a composition of the disclosure and a low dose of a GVHD prophylactic agent (for example, a single GVHD prophylactic agent at a low dose) develop moderate to severe cGVHD. In some embodiments, less than about 5% of subjects that are administered a composition of the disclosure and a low dose of a GVHD prophylactic agent (for example, a single GVHD prophylactic agent at a low dose) develop moderate to severe cGVHD.

Subjects administered a composition of the disclosure (e.g., a cell population as described herein) and a low dose of a GVHD prophylactic agent (for example, a single GVHD prophylactic agent at a low dose) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop severe cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop severe cGVHD. In some embodiments, less than about 40% of subjects that are administered a composition of the disclosure and a low dose of a GVHD prophylactic agent (for example, a single GVHD prophylactic agent at a low dose) develop severe cGVHD. In some embodiments, less than about 30% of subjects that are administered a composition of the disclosure and a low dose of a GVHD prophylactic agent (for example, a single GVHD prophylactic agent at a low dose) develop severe cGVHD. In some embodiments, less than about 20% of subjects that are administered a composition of the disclosure and a low dose of a GVHD prophylactic agent (for example, a single GVHD prophylactic agent at a low dose) develop severe cGVHD. In some embodiments, less than about 15% of subjects that are administered a composition of the disclosure and a low dose of a GVHD prophylactic agent (for example, a single GVHD prophylactic agent at a low dose) develop severe cGVHD. In some embodiments, less than about 10% of subjects that are administered a composition of the disclosure and a low dose of a GVHD prophylactic agent (for example, a single GVHD prophylactic agent at a low dose) develop severe cGVHD. In some embodiments, less than about 5% of subjects that are administered a composition of the disclosure and a low dose of a GVHD prophylactic agent (for example, a single GVHD prophylactic agent at a low dose) develop severe cGVHD. In some embodiments, less than about 3% of subjects that are administered a composition of the disclosure and a low dose of a GVHD prophylactic agent (for example, a single GVHD prophylactic agent at a low dose) develop severe cGVHD. In some embodiments, less than about 2% of subjects that are administered a composition of the disclosure and a low dose of a GVHD prophylactic agent (for example, a single GVHD prophylactic agent at a low dose) develop severe cGVHD. In some embodiments, less than about 1% of subjects that are administered a composition of the disclosure and a low dose of a GVHD prophylactic agent (for example, a single GVHD prophylactic agent at a low dose) develop severe cGVHD.

A low dose of a GVHD prophylactic agent can be, for example a target trough level of less than about 25 ng/mL, less than about 20 ng/mL, less than about 15 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a low dose of a GVHD prophylactic is a target trough level of approximately 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a low dose of a GVHD prophylactic agent is tacrolimus with a target trough level of approximately 5 ng/mL to approximately 10 ng/mL. In some embodiments, a low dose of a GVHD prophylactic agent is tacrolimus with a target trough level of approximately 4 ng/mL to approximately 6 ng/mL.

In some embodiments, a low dose of a GVHD prophylactic agent is sirolimus with a target trough level of approximately 3 ng/mL to approximately 8 ng/mL. In some embodiments, a low dose of a GVHD prophylactic agent is sirolimus with a target trough level of approximately 4 ng/mL to approximately 8 ng/mL.

The low dose GVHD prophylactic agent (for example, single GVHD prophylactic agent at a low dose) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the low dose GVHD prophylactic agent (for example, single GVHD prophylactic agent at a low dose) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### Organ-Specific GVHD Stage Incidence

Subjects administered a composition of the disclosure (e.g., a cell population as described herein) exhibit a low incidence of ≥stage 1 GVHD signs for the skin, liver, gut, or a combination thereof, for example, a lower incidence compared to subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure exhibit ≥stage 1 GVHD signs for the skin, liver, gut, or a combination thereof.

Subjects administered a composition of the disclosure (e.g., a cell population as described herein) exhibit a low incidence of ≥stage 2 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 1 GVHD signs for the skin, liver, gut, or a combination thereof.

Subjects administered a composition of the disclosure (e.g., a cell population as described herein) exhibit a low incidence of ≥stage 3 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 3 GVHD signs for the skin, liver, gut, or a combination thereof.

Subjects administered a composition of the disclosure (e.g., a cell population as described herein) exhibit a low incidence of ≥stage 4 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 4 GVHD signs for the skin, liver, gut, or a combination thereof.

The incidence of the organ-specific GVHD signs can be assessed after a suitable amount of time elapses post-transplant, for example, about 20 days, about 21 days, about 25 days, about 28 days, about 30 days, about 35 days, about 40 days, about 42 days, about 45 days, about 49 days, about 50 days, about 55 days, about 56 days, about 60 days, about 63 days, about 65 days, about 70 days, about 75 days, about 77 days, about 80 days, about 84 days, about 85 days, about 90 days, about 91 days, about 95 days, about 98 days, about 100 days, about 105 days, about 110 days, about 112 days, about 115 days, about 119 days, about 120 days, about 150 days, about 200 days, about 365 days, about 1.5 years, about 2 years, about 2.5 years, about 3 years, about 3.5 years, about 4 years, about 4.5 years, or about 5 years post-transplant.

The incidence of the organ-specific GVHD signs can be calculated based on a population of, for example, at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.5 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

### Other Clinical Outcomes

### Survival, Hospitalization, and Relapse

In some embodiments, patient treated or provided with compositions, multi-component pharmaceutical treatments, multi-component cellular therapy products, cell populations, solutions, formulations, kits, and/or methods has a reduced risk of at least one of malignancy relapse, infection or renal failure.

A population of subjects treated or provided with compositions, multi-component pharmaceutical treatments, multi-component cellular therapy products, cell populations, solutions, formulations, kits, and/or methods of the disclosure (e.g., a cell population comprising a population of cells described herein) exhibit a high overall survival rate, for example, a higher overall survival rate compared to subjects that are administered an alternate composition. In some embodiments, at least about 40%, at least about 50%, at least about 55%, at least about 60%, at least about 65%, at least about 70%, at least about 75%, at least about 80%, at least about 85%, at least about 90%, or at least about 95% of subjects that are administered a composition of the disclosure are alive after about 1 year, about 1.5 years, about 2 years, about 2.5 years, about 3 years, about 3.5 years, about 4 years, about 4.5 years, about 5 years, about 7 years, about 10 years, about 15 years, about 20 years, about 30 years post-transplant.

A population of subjects treated or provided with compositions, multi-component pharmaceutical treatments, multi-component cellular therapy products, cell populations, solutions, formulations, kits, and/or methods of the disclosure (e.g., a cell population comprising a population of cells described herein) exhibit a low treatment-associated mortality rate, for example, a lower treatment-associated mortality rate compared to subjects that are administered an alternate composition. In some embodiments, a treatment-associated mortality rate of a population of subjects administered a composition of the disclosure is less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 25%, less than about 30%, less than about 35%, less than about 40%, or less than about 50% when evaluated after a suitable amount of time post-transplant, for example at approximately 6 months, about 1 year, about 1.5 years, about 2 years, about 2.5 years, about 3 years, about 3.5 years, about 4 years, about 4.5 years, or about 5 years post-transplant. In some embodiments a treatment-associated mortality rate of a population of subjects administered a composition of the disclosure is less than about 5% when evaluated at 1-year post-transplant. In some embodiments a treatment-associated mortality rate of a population of subjects administered a composition of the disclosure is less than about 1% when evaluated at 1-year post-transplant.

A population of subjects treated or provided with compositions, multi-component pharmaceutical treatments, multi-component cellular therapy products, cell populations, solutions, formulations, kits, and/or methods of the disclosure (e.g., a cell population comprising a population of cells described herein) exhibit GVHD free and relapse free survival (GRFS) rate, for example, a higher GRFS rate compared to subjects that are administered an alternate composition. In some embodiments, GRFS can refer to survival without relapse or Grade ≥3 aGVHD or extensive (e.g., severe) cGVHD. In some embodiments, GRFS can refer to survival without relapse or Grade ≥2 aGVHD or extensive (e.g., moderate to severe) cGVHD. In some embodiments, GRFS can refer to survival with no GVHD symptoms. In some embodiments, a GRFS rate of a population of subjects administered a composition of the disclosure is at least about 20%, at least about 30%, at least about 40%, at least about 50%, at least about 55%, at least about 60%, at least about 65%, at least about 70%, at least about 75%, at least about 80%, at least about 85%, at least about 90%, or at least about 95% when evaluated after a suitable amount of time post-transplant, for example at approximately 6 months, about 1 year, about 1.5 years, about 2 years, about 2.5 years, about 3 years, about 3.5 years, about 4 years, about 4.5 years, or about 5 years post-transplant. In some embodiments, a GRFS rate of a population of subjects administered a composition of the disclosure is at least about 60% when evaluated at 1-year post-transplant. In some embodiments, a GRFS rate of a population of subjects administered a composition of the disclosure is at least about 75% when evaluated at 1-year post-transplant. In some embodiments, a GRFS rate of a population of subjects administered a composition of the disclosure is more than 75% when evaluated at 3 years post-transplant. In some embodiments, a GRFS rate of a population of subjects administered a composition of the disclosure is more than 75% when evaluated at 5 years post-transplant. In some embodiments, a GRFS rate of a population of subjects administered a composition of the disclosure is more than 75% when evaluated at 7 years post-transplant. In some embodiments, a GRFS rate of a population of subjects administered a composition of the disclosure is more than 75% when evaluated at 10 years post-transplant.

A population of subjects treated or provided with compositions, multi-component pharmaceutical treatments, multi-component cellular therapy products, cell populations, solutions, formulations, kits, and/or methods of the disclosure (e.g., a cell population comprising a population of cells described herein) exhibit a short time to discharge from hospital, for example, a shorter time to discharge from hospital compared to subjects that are administered an alternate composition. In some embodiments, the average time to discharge after day 0 of a transplantation regimen is less than about 20 days, less than about 19 days, less than about 18 days, less than about 17 days, less than about 16 days, less than about 15 days, less than about 14 days, less than about 13 days, less than about 12 days, less than about 11 days, less than about 10 days, less than about 9 days, or less than about 8 days. In some embodiments, the average time to discharge after day 0 of a transplantation regimen is less than about 17 days. In some embodiments, the average time to discharge after day 0 of a transplantation regimen is less than about 18 days. In embodiments, the human subject treated or provided with compositions, multi-component pharmaceutical treatments, multi-component cellular therapy products, cell populations, solutions, formulations, kits, and/or methods as disclosed herein have no relapse of their malignancy about one year after being administered the pharmaceutical dosing regimen.

A population of subjects treated or provided with compositions, multi-component pharmaceutical treatments, multi-component cellular therapy products, cell populations, solutions, formulations, kits, and/or methods of the disclosure (e.g., a cell population comprising a population of cells described herein) exhibit a low relapse rate, for example, a lower relapse rate compared to subjects that are administered an alternate composition. In some embodiments, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, less than about 40%, less than about 45%, less than about 50%, less than about 55%, or less than about 60% of subjects that are administered a composition of the disclosure relapse within a suitable amount of time post-transplant, for example at approximately 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant. In some embodiments, less than about 35% of subjects that are administered a composition of the disclosure relapse within 1-year post-transplant. In some embodiments, less than about 25% of subjects that are administered a composition of the disclosure relapse within 1-year post-transplant.

A population of subjects that do not have active disease when treated or provided with compositions, multi-component pharmaceutical treatments, multi-component cellular therapy products, cell populations, solutions, formulations, kits, and/or methods of the disclosure (e.g., a cell population as described herein) exhibit a low relapse rate, for example, a lower relapse rate compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, less than about 40%, less than about 45%, less than about 50%, less than about 55%, or less than about 60% of subjects that do not have active disease when administered a composition of the disclosure relapse within a suitable amount of time post-transplant, for example at approximately 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant.

In some cases, the patient treated or provided with compositions, multi-component pharmaceutical treatments, multi-component cellular therapy products, cell populations, solutions, formulations, kits, and/or methods has no relapse of their malignancy about one year after being administered the cell populations.

A population of subjects that are in complete remission when administered a composition of the disclosure (e.g., a cell population as described herein) exhibit a low relapse rate, for example, a lower relapse rate compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, or less than about 40% of subjects that are in complete remission when administered a composition of the disclosure relapse within a suitable amount of time post-transplant, for example at approximately 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant.

In some cases, the patient has no GHVD or relapse of their malignancy one year after being administered the cell populations.

In some embodiments, subjects administered a composition of the disclosure (e.g., a cell population as described herein) the subject experiences GFRS for approximately 11 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, GFRS can refer to survival free of death from any cause, relapse, Grade 3-4 aGVHD (e.g., graded per MAGIC), and moderate to severe cGVHD (e.g., graded per NIH consensus criteria). In some embodiments, relapse for acute leukemia can refer to ≥5% blasts in the bone marrow or peripheral blood, reappearance of pre-transplant cytogenetic abnormality, and/or new evidence or redevelopment of extramedullary disease. In some embodiments, relapse for MDS can refer to satisfying criteria for evolution into acute leukemia, reappearance of pre-transplant morphologic abnormalities, detected in bone marrow specimens, and/or reappearance of pre-transplant cytogenetic abnormality in at least one metaphase on each of two separate consecutive examinations at least one month apart, regardless of the number of metaphases analyzed.

In some embodiments, relapse for CML can refer to the emergence of BCR-ABL positivity and/or cytogenetic relapse that requires the institution of secondary therapy for CML. In some embodiments, this can include the use of donor lymphocyte infusion, tyrosine kinase inhibitors (TKIs), or chemotherapy in response to molecular and/or cytogenetic progression.

In some embodiments, subjects administered a composition of the disclosure (e.g., a cell population as described herein) experience increased relapse-free survival, relative to a corresponding human subject that has been administered a standard myeloablative allogeneic hematopoietic stem cell transplant (alloHSCT). In some embodiments, the subject experiences relapse-free survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, subjects administered a composition of the disclosure (e.g., a cell population as described herein) experience increased overall survival, relative to a corresponding human subject that has been administered a standard myeloablative allogeneic hematopoietic stem cell transplant (alloHSCT). In some embodiments, the subject experiences overall survival for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, the subject does not experience non-relapse mortality for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, the subject does not experience Grade 3 or higher infection for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

In some embodiments, the Grade 3 or higher infection is graded using the BMT-CTN MOP V4.0 grading scale.

In some embodiments, subjects administered a composition of the disclosure (e.g., a cell population as described herein) have a decreased incidence of re-hospitalization, relative to a corresponding human subject that has been administered a standard myeloablative allogeneic hematopoietic stem cell transplant (alloHSCT). In some embodiments, the subject does not require re-hospitalization for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

### Engraftment and Immune Reconstitution

### Graft Failure

A population of subjects that are administered a composition of the disclosure (e.g., a cell population as described herein) exhibit a low rate of primary graft failure, for example, a lower rate of primary graft failure compared to subjects that are administered an alternate composition. Primary graft failure can be a failure to achieve an absolute neutrophil count of >500 cells/μL after Day 30 post-transplant. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 30%, or less than about 40% of subjects administered a composition of the disclosure exhibit primary graft failure. In some embodiments, less than about 1% of subjects administered a composition of the disclosure exhibit primary graft failure. In some embodiments, less than about 3% of subjects administered a composition of the disclosure exhibit primary graft failure.

A population of subjects that are administered a composition of the disclosure (e.g., a cell population as described herein) exhibit a low rate of secondary graft failure, for example, a lower rate of secondary graft failure compared to subjects that are administered an alternate composition. Secondary graft failure can be a sustained loss of hematopoiesis after engraftment. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 30%, or less than about 40% of subjects administered a composition of the disclosure exhibit secondary graft failure when evaluated a suitable amount of time post-transplant, for example at approximately 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant. In some embodiments, less than about 1% of subjects administered a composition of the disclosure exhibit secondary graft failure within 1-year post-transplant. In some embodiments, less than about 5% of subjects administered a composition of the disclosure exhibit secondary graft failure within 1-year post-transplant.

In some embodiments, subjects administered a composition of the disclosure (e.g., a cell population as described herein) have a decreased wherein incidence of primary graft failure or secondary graft failure, relative to a corresponding human subject that has been administered a standard myeloablative allogeneic hematopoietic stem cell transplant (alloHSCT). In some embodiments, the subject is free of primary graft failure or secondary graft failure for approximately 1 month or more, approximately 2 months or more, approximately 3 months or more, approximately 4 months or more, approximately 5 months or more, approximately 6 months or more, approximately 7 months or more, approximately 8 months or more, approximately 9 months or more, approximately 10 months or more, approximately 11 months or more, approximately 1 year or more, approximately 1.25 years or more, approximately 1.50 years or more, approximately 1.75 years or more, approximately 2 years or more, approximately 2.25 years or more, approximately 2.50 years or more, approximately 2.75 years or more, approximately 3 years or more, approximately 3.25 years or more, approximately 3.50 years or more, approximately 3.75 years or more, approximately 4 years or more, approximately 4.25 years or more, approximately 4.50 years or more, approximately 4.75 years or more, or approximately 5 years or more after administration of the composition of the disclosure.

### Neutrophil Engraftment

A population of subjects that are administered a composition of the disclosure (e.g., a cell population as described herein) exhibit fast neutrophil engraftment, for example, faster neutrophil engraftment compared to subjects that are administered an alternate composition. Neutrophil engraftment can be indicated by a sustained neutrophil count of >500 cells/μL in the peripheral blood of the recipient. In some embodiments, a population of subjects administered a composition of the disclosure achieve neutrophil engraftment by a median of approximately 7 days, about 8 days, about 9 days, about 10 days, about 11 days, about 12 days, about 12 days, about 14 days, or about 15 days post-transplant. In some embodiments, a population of subjects administered a composition of the disclosure achieve neutrophil engraftment by a median of 13 days post-transplant. In some embodiments, a population of subjects administered a composition of the disclosure achieve neutrophil engraftment by a median of 12 days post-transplant. In some embodiments, a population of subjects administered a composition of the disclosure achieve neutrophil engraftment by a median of 11 days post-transplant.

In some embodiments, subjects administered a composition of the disclosure (e.g., a cell population as described herein) have increased incidence, timing, or both incidence and timing of neutrophil engraftment, relative to a corresponding human subject that has been administered a standard myeloablative allogeneic hematopoietic stem cell transplant (alloHSCT). In some embodiments, neutrophil engraftment occurs approximately 1 day or more, approximately 2 days or more, approximately 3 days or more, approximately 4 days or more, approximately 5 days or more, approximately 6 days or more, approximately 7 days or more, approximately 8 days or more, approximately 9 days or more, approximately 10 days or more, approximately 15 days or more, approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, or approximately 90 days or more after administration of the composition of the disclosure.

### Platelet Engraftment

A population of subjects that are administered a composition of the disclosure (e.g., a cell population comprising a population of cells described herein) exhibit fast platelet engraftment, for example, faster platelet engraftment compared to subjects that are administered an alternate composition. Platelet engraftment can be indicated by a platelet count >20,000/mm³ for 3 consecutive days without platelet transfusion in the peripheral blood of the recipient. In some embodiments, a population of subjects administered a composition of the disclosure achieve platelet engraftment by a median of approximately 7 days, about 8 days, about 9 days, about 10 days, about 11 days, about 12 days, about 12 days, about 14 days, or about 15 days post-transplant. In some embodiments, a population of subjects administered a composition of the disclosure achieve platelet engraftment by a median of 13 days post-transplant. In some embodiments, a population of subjects administered a composition of the disclosure achieve platelet engraftment by a median of 12 days post-transplant. In some embodiments, a population of subjects administered a composition of the disclosure achieve platelet engraftment by a median of 11 days post-transplant.

In some embodiments, subjects administered a composition of the disclosure (e.g., a cell population as described herein) have increased incidence, timing, or both incidence and timing of platelet engraftment, relative to a corresponding human subject that has been administered a standard myeloablative allogeneic hematopoietic stem cell transplant (alloHSCT). In some embodiments, platelet engraftment occurs approximately 1 day or more, approximately 2 days or more, approximately 3 days or more, approximately 4 days or more, approximately 5 days or more, approximately 6 days or more, approximately 7 days or more, approximately 8 days or more, approximately 9 days or more, approximately 10 days or more, approximately 15 days or more, approximately 20 days or more, approximately 25 days or more, approximately 30 days or more, approximately 35 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, or approximately 90 days or more after administration of the composition of the disclosure.

### T Cell Engraftment

A population of subjects that are administered a composition of the disclosure (e.g., a cell population as described herein) exhibit a high proportion of circulating Tregs, for example, a higher proportion of circulating Tregs compared to subjects that are administered an alternate composition. In some embodiments, an average of at least about 5%, at least about 7.5%, at least about 10%, at least about 12.5%, at least about 15%, at least about 16%, at least about 17%, at least about 18%, at least about 19%, at least about 20%, at least about 21%, at least about 22%, at least about 23%, at least about 24%, or at least about 25% of circulating CD4⁺ T cells are Tregs when subjects are evaluated a suitable amount of time post-transplant, for example, at approximately 14 days, 15 days, 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 110 days, 120 days, 130 days, 140 days, 150 days, 160 days, 170 days, or 180 days post-transplant. In some embodiments, an average of at least about 15% of circulating CD4⁺ T cells are Tregs when subjects are evaluated 28 days post-transplant.

A population of subjects that are administered a composition of the disclosure (e.g., a cell population as described herein) exhibit a normal CD4:CD8 T cell ratio, for example, a higher CD4:CD8 T cell ratio compared to subjects that are administered an alternate composition or normal healthy control subjects.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥0.8 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant. In some embodiments, at least 50% of subjects have a CD4:CD8 T cell ratio of ≥1 when evaluated 28 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.2 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.5 when evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

A population of subjects that are administered a composition of the disclosure (e.g., a cell population as described herein) exhibit a high proportion of donor-derived circulating T cells at an early timepoint after transplant, for example, a higher proportion of donor-derived circulating T cells compared to subjects that are administered an alternate composition.

In some embodiments, more than 50%, more than 60%, more than 70%, more than 80%, or more than 90% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

### B Cell Engraftment

A population of subjects that are administered a composition of the disclosure (e.g., a cell population as described herein) exhibit a high concentration of circulating B cells at an early timepoint after transplant, for example, a higher concentration of circulating B cells compared to subjects that are administered an alternate composition. Achieving a high concentration of circulating B cells at an early timepoint after transplant can be important for immunocompetence, and can allow vaccines to elicit protective immune responses at an earlier timepoint post-transplant.

In some embodiments, more than about 50 B cells per μL, more than about 60 B cells per μL, more than about 70 B cells per μL, more than about 80 B cells per μL, more than about 90 B cells per μL, more than about 100 B cells per μL, or more than about 110 B cells per μL are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 50 B cells per μL, more than 60 B cells per μL, more than 70 B cells per μL, more than 80 B cells per μL, more than 90 B cells per μL, more than 100 B cells per μL, or more than 110 B cells per μL are present in the blood of at least 75% of subjects evaluated about 60 days post-transplant.

A population of subjects that are administered a composition of the disclosure (e.g., a cell population as described herein) exhibit a high proportion of mature B cells at an early timepoint after transplant, for example, a higher proportion of circulating B cells that are mature B cells (e.g., IgD⁺ and/or CD27⁺) compared to subjects that are administered an alternate composition. Achieving a high proportion of mature B cells at an early timepoint after transplant can be important for immunocompetence, and can allow vaccines to elicit protective immune responses at an earlier timepoint post-transplant.

In some embodiments, at least about 50%, at least about 60%, at least about 70%, at least about 80%, or at least about 90% of circulating CD19⁺ B cells are IgD⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 25%, at least about 30%, at least about 35%, at least about 40%, or at least about 45% of circulating CD19⁺ B cells are CD27⁺ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at approximately 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

Clinical outcomes disclosed herein can be calculated based on a population of, for example, at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

### Kits

Another aspect provides a kit that comprises a solution comprising a first container comprising a first population of CD45⁺ cells (e.g., HSPCc), a second container comprising a solution comprising a third population of CD45⁺ cells (e.g., Tregs), a third container comprising a solution comprising a population of cells enriched for regulatory T cells (Tregs), and a fourth container comprising a solution comprising one or more cells comprising a chimeric receptor. In this aspect, the solution comprising the first population of CD45⁺ cells, the solution comprising the third population of CD45⁺ cells, the solution comprising the population of cells enriched for Tregs, and the solution comprising the one or more cells comprising a chimeric receptor are as defined according to any herein disclosed multi-component pharmaceutical treatment or method. In various embodiments, the kit further comprises a fourth container comprising the GVHD prophylactic agent. In some cases, the further comprising instructions for performing any herein-disclosed method.

A further aspect provides a kit comprising: (a) one or more reagents to sort CD34⁺ cells from a mobilized peripheral blood composition; (b) one or more reagents to sort regulatory T cells (Tregs) from the mobilized peripheral blood composition; (c) one or more reagents to detect a number of CD3⁺ conventional T cells in the mobilized peripheral blood; and (d) a solution comprising one or more doses of a graft vs host disease (GVHD) prophylactic agent. In some embodiments, the kit further comprises instructions for performing any herein-disclosed method.

Various embodiments provide a kit comprising one or more reagents for sorting HSPCs, for instance, a kit may comprise reagents to enrich a CD34⁺ cell population from a mobilized peripheral blood donation.

Some embodiments provide a kit comprising one or more reagents for sorting Tregs, for instance, a kit may comprise reagents to enrich a Treg cell population (using markers as described elsewhere herein) from a mobilized peripheral blood donation.

Embodiments provides a kit comprising one or more conditioning reagents for a conditioning regimen, for instance, a kit may comprise reagents for myeloablation or myeloreduction of a recipient.

Some embodiments provide a kit comprising one or more GVHD prophylactic agents.

In any herein-disclosed method, the method further comprises providing instructions for use (IFU), the IFU including instructions for administering the cell populations to the patient. In some cases, the IFU also include instructions for administering one or more pharmaceutical agents or compositions to the patient.

### Definitions

The terminology used herein is for the purpose of describing particular cases only and is not intended to be limiting.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used in either the present Specification and/or in the claims, are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

By “one or more” or “at least one” is meant at least one, e.g., one, two, three, four, five, six, seven, eight, nine, ten or more.

The term “similar” is understood to be resembling up to and including identical. Therefore two (or more) items may be identical, substantially identical, comprise equivalent components, comprise substitutable components, comprise analogous components, comprise comparable components, comprise complementary components, comprise related components, comprise like components, and/or the differences between the two (or more items) are insubstantial and/or result in a composition having equivalent properties, identical properties, substantially identical properties, and the like.

The term “treatment” (and grammatical variations thereof such as “treat” or “treating”) can refer to complete or partial amelioration or reduction of a disease or condition or disorder, or a symptom, adverse effect or outcome, or phenotype associated therewith. Desirable effects of treatment may include, without limitation, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

The term “preventing,” includes providing prophylaxis with respect to the occurrence or recurrence of a disease in a subject that may be predisposed to the disease but has not yet been diagnosed with the disease. In some embodiments, the provided compositions, treatments, and therapies are used to delay development of a disease or to slow the progression of a disease.

The terms “patient” and “subject” are synonyms.

Any aspect or embodiment described herein can be combined with any other aspect or embodiment as disclosed herein.

### Additional Aspects

A further aspect provides a method of treating a human subject comprising a step (a) of administering a plurality of populations of cells, in which the plurality of populations of cells comprises: (i). a population of hematopoietic stem and progenitor cells (HSPCs); (ii) a population of cells comprising regulatory T cells (Tregs); and (iii) a population of conventional T cells (Tcons); and a step (b) of administering no more than one graft versus host disease (GVHD) prophylactic agent for less than about 120 days. In this method, the population of HSPCs comprises less than about 2% CD3⁺ cells.

An additional aspect is a method of transplanting a conventional T cell (Tcon) population into a human subject without eliciting a stage 2 or higher graft versus host disease (GVHD) response up to approximately 100 days after transplanting. The method comprising: (i). administering a solution comprising a population of conventional T cells (Tcons); and (ii). administering a solution comprising a population of regulatory T cells (Tregs). In this method, the population of Tcons is cryopreserved for at least about 4 hours.

A yet another aspect provides a method of treating a hematologic malignancy in a human subject in need thereof, the method comprising administering to the human subject: (a) a population of hematopoietic stem and progenitor cells (HSPCs); (b) a population of regulatory T cells (Tregs); and (c) a population of conventional T cells (Tcons). In the method, the population of HSPCs and the population of Tregs are administered prior to the population of Tcons; and peripheral blood of the human subject exhibits an elevated Treg count until about 100 days after the administering the three populations of cells as compared to a healthy human subject that was not administered the three populations of cells.

A further aspect provides a method of transplanting a conventional T cell (Tcon) population into a human subject without eliciting a stage 2 or higher graft versus host disease (GVHD) response up to approximately 100 days after transplanting. The method comprising (i). administering a population of conventional T cells (Tcons); and (ii). administering a population of regulatory T cells (Tregs). In this method, the population of Tcons is administered at least about 12 hours after the population of Tregs is administered.

### ENUMERATED EMBODIMENTS

The following enumerated embodiments describe further non-limiting aspects of the present disclosure.

Embodiment 1. A method of treating a human subject having or suspected of having a malignancy, comprising administering to the human subject a cellular therapy,
wherein the cellular therapy comprises:
(a) one or more allogeneic cells comprising a chimeric receptor; and
(b) an immune-modulating cell therapy product,
wherein the one or more allogeneic cells and/or one or more cells of the immune-modulating cell therapy product are from an HLA-compatible donor, relative to the human subject.

Embodiment 2. The method of embodiment 1, wherein the HLA-compatible donor is an allogeneic HLA-matched donor, relative to the human subject.

Embodiment 3. The method of embodiment 1, wherein the HLA-compatible donor is an allogeneic HLA-mismatched donor, relative to the human subject.

Embodiment 4. The method of any one of embodiments 1-3, wherein the one or more allogeneic cells have not been genetically modified to reduce risk of graft versus host disease (GVHD) and/or to prevent allogeneic rejection with respect to the human subject.

Embodiment 5. The method of any one of embodiments 1-4, wherein the immune-modulating cell therapy product comprises a regulatory T cell therapy.

Embodiment 6. The method of any one of embodiments 1-4, wherein the immune-modulating cell therapy product comprises an allogeneic hematopoietic stem cell transplant (HSCT).

Embodiment 7. The method of embodiment 6, wherein the allogeneic HSCT comprises a population of CD34+ hematopoietic stem and progenitor cells (HSPCs).

Embodiment 8. The method of embodiment of embodiment 6 or embodiment 7, wherein the allogeneic HSCT comprises a population of CD4+CD25+CD127dim regulatory T cells (Tregs).

Embodiment 9. The method of any one of embodiments 6-8, wherein the allogeneic HSCT comprises a population of HSPCs and a population of Tregs.

Embodiment 10. The method of any one of embodiments 6-9, wherein the allogeneic HSCT comprises a population of CD45RA− memory T cells (Tmems).

Embodiment 11. The method of any one of embodiments 6-10, wherein the allogeneic HSCT comprises a population of HSPCs and a population of Tmems.

Embodiment 12. The method of any one of embodiments 6-10, wherein the allogeneic HSCT comprises a population of Treg and a population of Tmems.

Embodiment 13. The method of any one of embodiments 6-12, wherein the allogeneic HSCT comprises a population of HSPCs, a population of Treg, and a population of Tmems.

Embodiment 14. The method of any one of embodiments 10-13, wherein the allogeneic HSCT has been depleted of naïve conventional CD3+CD25−CD45RA+ T cells.

Embodiment 15. The method of any one of embodiments 6-14, wherein the allogeneic HSCT comprises a population of conventional CD3+ T cells (Tcons).

Embodiment 16. The method of any one of embodiments 6-15, wherein allogeneic HSCT comprises a population of HSPCs and a population of Tcons.

Embodiment 17. The method of any one of embodiments 6-15 wherein allogeneic HSCT comprises a population of Tregs and a population of Tcons.

Embodiment 18. The method of any one of embodiments 6-15 wherein allogeneic HSCT comprises a population of Tmems and a population of Tcons.

Embodiment 19. The method of any one of embodiments 6-17, wherein allogeneic HSCT comprises a population of HSPCs, a population of Tregs, and a population of Tcons.

Embodiment 20. The method of any one of embodiments 6-19, wherein allogeneic HSCT comprises a population of HSPCs, a population of Tregs, a population of Tmems, and a population of Tcons.

Embodiment 21. The method of embodiment 6, wherein the allogeneic HSCT is a T cell-depleted allogeneic HSCT.

Embodiment 22. The method of any one of embodiments 1-21, wherein the cellular therapy reduces incidence of transplant related mortality (TRM), as compared to a human subject administered only the one or more allogeneic cells comprising a chimeric receptor.

Embodiment 23. The method of any one of embodiments 1-22, wherein the cellular therapy reduces incidence and/or severity of graft vs host disease (GVHD), as compared to a human subject administered only the one or more allogeneic cells comprising a chimeric receptor.

Embodiment 24. The method of any one of embodiments 1-23, wherein the cellular therapy reduces incidence and/or severity of cytokine release syndrome (CRS), as compared to a human subject administered only the one or more allogeneic cells comprising a chimeric receptor.

Embodiment 25. The method of any one of embodiments 1-24, wherein the cellular therapy reduces incidence and/or severity of immune effector cell-associated neurotoxicity syndrome (ICANS), as compared to a human subject administered only the one or more allogeneic cells comprising a chimeric receptor.

Embodiment 26. The method of any one of embodiments 1-25, wherein the one or more cells comprising the chimeric receptor comprise a pharmaceutical composition comprising an isolated nucleic acid encoding the chimeric receptor.

Embodiment 27. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs); and
(b) one or more cells comprising a chimeric receptor or a pharmaceutical composition comprising an isolated nucleic acid encoding the chimeric receptor.

Embodiment 28. The method of embodiment 27, wherein the multi-component cellular therapy further comprises a population of CD4+CD25+CD127dim regulatory T cells (Tregs).

Embodiment 29. The method of embodiment 28, wherein the population of Tregs comprises the one or more cells comprising a chimeric receptor.

Embodiment 30. The method of any one of embodiments 27-29, wherein the multi-component cellular therapy further comprises a population of CD45RA− memory T cells (Tmems).

Embodiment 31. The method of embodiment 30, wherein the population of Tmems comprises the one or more cells comprising a chimeric receptor.

Embodiment 32. The method of any one of embodiments 27-31, wherein the multi-component cellular therapy further comprises a population of conventional CD3+ T cells (Tcons).

Embodiment 33. The method of embodiment 32, wherein the population of Tcons comprises the one or more cells comprising a chimeric receptor.

Embodiment 34. A method of treating a human subject having or suspected of having a hematologic malignancy, comprising administering to the human subject a multi-component cellular therapy, wherein the multi-component cellular therapy comprises a population of CD34+ hematopoietic stem and progenitor cells (HSPCs), and wherein the population of HSPCs comprises one or more cells comprising a chimeric receptor.

Embodiment 35. The method of embodiment 34, wherein the multi-component cellular therapy further comprises a population of CD4+CD25+CD127dim regulatory T cells (Tregs).

Embodiment 36. The method of embodiment 34 or embodiment 35, wherein the multi-component cellular therapy further comprises a population of CD45RA− memory T cells (Tmems).

Embodiment 37. The method of any one of embodiments 34-36, wherein the multi-component cellular therapy further comprises a population of conventional CD3+ T cells (Tcons).

Embodiment 38. A method of treating a human subject having or suspected of having a hematologic malignancy, comprising administering to the human subject a multi-component cellular therapy, wherein the multi-component cellular therapy comprises:
(a) a population of CD4+CD25+CD127dim regulatory T cells (Tregs); and
(b) one or more cells comprising a chimeric receptor or a pharmaceutical composition comprising an isolated nucleic acid encoding the chimeric receptor.

Embodiment 39. The method of embodiment 38, wherein the multi-component cellular therapy further comprises a population of CD34+ hematopoietic stem and progenitor cells (HSPCs).

Embodiment 40. The method of embodiment 39, wherein the population of HSPCs comprises the one or more cells comprising a chimeric receptor.

Embodiment 41. The method of any one of embodiments 38-40, wherein the multi-component cellular therapy further comprises a population of CD45RA− memory T cells (Tmems).

Embodiment 42. The method of embodiment 41, wherein the population of Tmems comprises the one or more cells comprising a chimeric receptor.

Embodiment 43. The method of any one of embodiments 38-42, wherein the multi-component cellular therapy further comprises a population of conventional CD3+ T cells (Tcons).

Embodiment 44. The method of embodiment 43, wherein the population of Tcons comprises the one or more cells comprising a chimeric receptor.

Embodiment 45. A method of treating a human subject having or suspected of having a hematologic malignancy, comprising administering to the human subject a multi-component cellular therapy, wherein the multi-component cellular therapy comprises a population of CD4+CD25+CD127dim regulatory T cells (Tregs), and wherein the population of Tregs comprises one or more cells comprising a chimeric receptor.

Embodiment 46. The method of embodiment 45, wherein the multi-component cellular therapy further comprises a population of CD34+ hematopoietic stem and progenitor cells (HSPCs).

Embodiment 47. The method of embodiment 45 or embodiment 46, wherein the multi-component cellular therapy further comprises a population of CD45RA− memory T cells (Tmems).

Embodiment 48. The method of any one of embodiments 45-47, wherein the multi-component cellular therapy further comprises a population of conventional CD3+ T cells (Tcons).

Embodiment 49. A method of treating a human subject having or suspected of having a hematologic malignancy, comprising administering to the human subject a multi-component cellular therapy, wherein the multi-component cellular therapy comprises:
(a) a population of CD45RA− memory T cells (Tmems); and
(b) one or more cells comprising a chimeric receptor or a pharmaceutical composition comprising an isolated nucleic acid encoding the chimeric receptor.

Embodiment 50. The method of embodiment 49, wherein the multi-component cellular therapy further comprises a population of CD34+ hematopoietic stem and progenitor cells (HSPCs).

Embodiment 51. The method of embodiment 50, wherein the population of HSPCs comprises the one or more cells comprising a chimeric receptor.

Embodiment 52. The method of any one of embodiments 49-51, wherein the multi-component cellular therapy further comprises a population of CD4+CD25+CD127dim regulatory T cells (Tregs).

Embodiment 53. The method of embodiment 52 wherein the population of Tregs comprises the one or more cells comprising a chimeric receptor.

Embodiment 54. The method of any one of embodiments 49-53, wherein the multi-component cellular therapy further comprises a population of conventional CD3+ T cells (Tcons).

Embodiment 55. The method of embodiment 54, wherein the population of Tcons comprises the one or more cells comprising a chimeric receptor.

Embodiment 56. A method of treating a human subject having or suspected of having a hematologic malignancy, comprising administering to the human subject a multi-component cellular therapy, wherein the multi-component cellular therapy comprises a population of CD45RA− memory T cells (Tmems), and wherein the population of Tmems comprises one or more cells comprising a chimeric receptor.

Embodiment 57. The method of embodiment 56, wherein the multi-component cellular therapy further comprises a population of CD34+ hematopoietic stem and progenitor cells (HSPCs).

Embodiment 58. The method of embodiment 56 or embodiment 57 wherein the multi-component cellular therapy further comprises a population of CD4+CD25+CD127dim regulatory T cells (Tregs).

Embodiment 59. The method of any one of embodiments 56-58, wherein the multi-component cellular therapy further comprises a population of conventional CD3+ T cells (Tcons).

Embodiment 60. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs);
(b) a population of CD4+CD25+CD127dim regulatory T cells (Tregs); and
(c) one or more cells comprising a chimeric receptor or a pharmaceutical composition comprising an isolated nucleic acid encoding the chimeric receptor.

Embodiment 61. The method of embodiment 49, wherein the population of HSPCs comprises the one or more cells comprising a chimeric receptor.

Embodiment 62. The method of embodiment 49, wherein the population of Tregs comprises the one or more cells comprising a chimeric receptor.

Embodiment 63. The method of any one of embodiments 49-62, wherein the multi-component cellular therapy further comprises a population of CD45RA− memory T cells (Tmems).

Embodiment 64. The method of embodiment 63, wherein the population of Tmems comprises the one or more cells comprising a chimeric receptor.

Embodiment 65. The method of any one of embodiments 49-64, wherein the multi-component cellular therapy further comprises a population of conventional CD3+ T cells (Tcons).

Embodiment 66. The method of embodiment 65, wherein the population of Tcons comprises the one or more cells comprising a chimeric receptor.

Embodiment 67. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs); and
(b) a population of CD4+CD25+CD127dim regulatory T cells (Tregs),
wherein the population of HSPCs, the population of Tregs, or the population of HSPCs and the population of Tregs comprises one or more cells comprising a chimeric receptor.

Embodiment 68. The method of embodiment 67, wherein the multi-component cellular therapy further comprises a population of CD45RA− memory T cells (Tmems).

Embodiment 69. The method of embodiment 67 or embodiment 68, wherein the multi-component cellular therapy further comprises a population of conventional CD3+ T cells (Tcons).

Embodiment 70. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs);
(b) a population of CD45RA− memory T cells (Tmems); and
(c) one or more cells comprising a chimeric receptor or a pharmaceutical composition comprising an isolated nucleic acid encoding the chimeric receptor.

Embodiment 71. The method of embodiment 70, wherein the population of HSPCs comprises the one or more cells comprising a chimeric receptor.

Embodiment 72. The method of embodiment 70, wherein the population of Tmems comprises the one or more cells comprising a chimeric receptor.

Embodiment 73. The method of any one of embodiments 70-72, wherein the multi-component cellular therapy further comprises a population of CD4+CD25+CD127dim regulatory T cells (Tregs).

Embodiment 74. The method of embodiment 73, wherein the population of Tregs comprises the one or more cells comprising a chimeric receptor.

Embodiment 75. The method of any one of embodiments 70-74, wherein the multi-component cellular therapy further comprises a population of conventional CD3+ T cells (Tcons).

Embodiment 76. The method of embodiment 75, wherein the population of Tcons comprises the one or more cells comprising a chimeric receptor.

Embodiment 77. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs); and
(b) a population of CD45RA− memory T cells (Tmems),
wherein the population of HSPCs, the population of Tmems, or the population of HSPCs and the population of Tmems comprises one or more cells comprising a chimeric receptor.

Embodiment 78. The method of embodiment 77, wherein the multi-component cellular therapy further comprises a population of CD4+CD25+CD127dim regulatory T cells (Tregs).

Embodiment 79. The method of embodiment 77 or embodiment 78, wherein the multi-component cellular therapy further comprises a population of conventional CD3+ T cells (Tcons).

Embodiment 80. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs);
(b) a population of conventional CD3+ T cells (Tcons); and
(c) one or more cells comprising a chimeric receptor or a pharmaceutical composition comprising an isolated nucleic acid encoding the chimeric receptor.

Embodiment 81. The method of embodiment 80, wherein the population of HSPCs comprises the one or more cells comprising a chimeric receptor.

Embodiment 82. The method of embodiment 80, wherein the population of Tcons comprises the one or more cells comprising a chimeric receptor.

Embodiment 83. The method of any one of embodiments 80-82, wherein the multi-component cellular therapy further comprises a population of CD4+CD25+CD127dim regulatory T cells (Tregs).

Embodiment 84. The method of embodiment 83, wherein the population of Tregs comprises the one or more cells comprising a chimeric receptor.

Embodiment 85. The method of any one of embodiments 80-84, wherein the multi-component cellular therapy further comprises a population of CD45RA− memory T cells (Tmems).

Embodiment 86. The method of embodiment 85, wherein the population of Tmems comprises the one or more cells comprising a chimeric receptor.

Embodiment 87. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs); and
(b) a population of conventional CD3+ T cells (Tcons),
wherein the population of HSPCs, the population of Tcons, or the population of HSPCs and the population of Tcons comprises one or more cells comprising a chimeric receptor.

Embodiment 88. The method of embodiment 87, wherein the multi-component cellular therapy further comprises a population of CD4+CD25+CD127dim regulatory T cells (Tregs).

Embodiment 89. The method of embodiment 87 or embodiment 88, wherein the multi-component cellular therapy further comprises a population of CD45RA− memory T cells (Tmems).

Embodiment 90. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs);
(b) a population of CD4+CD25+CD127dim regulatory T cells (Tregs);
(c) a population of conventional CD3+ T cells (Tcons); and
(d) one or more cells comprising a chimeric receptor or a pharmaceutical composition comprising an isolated nucleic acid encoding the chimeric receptor.

Embodiment 91. The method of embodiment 90, wherein the population of HSPCs comprises the one or more cells comprising a chimeric receptor.

Embodiment 92. The method of embodiment 90, wherein the population of Tregs comprises the one or more cells comprising a chimeric receptor.

Embodiment 93. The method of embodiment 90, wherein the population of Tcons comprises the one or more cells comprising a chimeric receptor.

Embodiment 94. The method of any one of embodiments 90-93, wherein the multi-component cellular therapy further comprises a population of CD45RA− memory T cells (Tmems).

Embodiment 95. The method of embodiment 94, wherein the population of Tmems comprises the one or more cells comprising a chimeric receptor.

Embodiment 96. A method of treating a human subject having or suspected of having a hematologic malignancy, comprising administering to the human subject a multi-component cellular therapy,
wherein the multi-component cellular therapy comprises:
(a) a population of CD34+ hematopoietic stem and progenitor cells (HSPCs);
(b) a population of CD4+CD25+CD127dim regulatory T cells (Tregs); and
(c) a population of conventional CD3+ T cells (Tcons),
wherein the population of HSPCs, the population of Tregs, and/or the population of Tcons comprises one or more cells comprising a chimeric receptor.

Embodiment 97. The method of embodiment 96, wherein the multi-component cellular therapy further comprises a population of CD45RA− memory T cells (Tmems).

Embodiment 98. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs);
(b) a population of CD4+CD25+CD127dim regulatory T cells (Tregs);
(c) a population of CD45RA− memory T cells (Tmems);
(d) a population of conventional CD3+ T cells (Tcons); and
(e) one or more cells comprising a chimeric receptor or a pharmaceutical composition comprising an isolated nucleic acid encoding the chimeric receptor.

Embodiment 99. The method of embodiment 98, wherein the population of HSPCs comprises the one or more cells comprising a chimeric receptor.

Embodiment 100. The method of embodiment 98, wherein the population of Tregs comprises the one or more cells comprising a chimeric receptor.

Embodiment 101. The method of embodiment 98, wherein the population of Tmems comprises the one or more cells comprising a chimeric receptor.

Embodiment 102. The method of embodiment 98, wherein the population of Tcons comprises the one or more cells comprising a chimeric receptor.

Embodiment 103. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs);
(b) a population of CD4+CD25+CD127dim regulatory T cells (Tregs);
(c) a population of CD45RA− memory T cells (Tmems); and
(d) a population of conventional CD3+ T cells (Tcons),
wherein the population of HSPCs, the population of Tregs, the population of Tmems, and/or the population of Tcons comprises one or more cells comprising a chimeric receptor.

Embodiment 104. The method of any one of embodiments 22, 26, 38, 49, 60, 70, 80, 90, and 98, wherein the pharmaceutical composition comprises a vector comprising the nucleic acid.

Embodiment 105. The method of embodiment 104, wherein the vector is a viral vector.

Embodiment 106. The method of embodiment 105, wherein the viral vector is selected from the group consisting of: a lentiviral vector, a retroviral vector, an oncolytic viral vector, an adenoviral vector, an adeno-associated viral (AAV) vector, and a virus-like particle (VLP).

Embodiment 107. The method of any one of embodiments 22, 26, 38, 49, 60, 70, 80, 90, and 98, wherein the pharmaceutical composition comprises a nanoparticle comprising the nucleic acid.

Embodiment 108. The method of embodiment 107, wherein the nanoparticle is a lipid nanoparticle (LNP).

Embodiment 109. The method of any one of embodiments 22, 26, 38, 49, 60, 70, 80, 90, and 98-108, wherein the pharmaceutical composition further comprises a pharmaceutically acceptable carrier, pharmaceutically acceptable excipient, or a combination thereof.

Embodiment 110. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs);
(b) a population of CD4+CD25+CD127dim regulatory T cells (Tregs); and
(c) a population of conventional CD3+ T cells (Tcons),
wherein the population of Tcons comprises one or more cells comprising a chimeric receptor.

Embodiment 111. The method of embodiment 110, wherein the multi-component cellular therapy further comprises a population of CD45RA− memory T cells (Tmems).

Embodiment 112. The method of embodiment 110 or embodiment 111, wherein the method further comprises genetically modifying one or more cells of the population of Tcons to express the chimeric receptor prior to administering the multi-component cellular therapy.

Embodiment 113. The method of embodiment 112, wherein the one or more cells are genetically modified via transfection with a vector comprising a nucleic acid encoding the chimeric receptor.

Embodiment 114. The method of embodiment 113, wherein the vector is a viral vector selected from the group consisting of a lentiviral vector, a retroviral vector, an oncolytic viral vector, an adenoviral vector, an adeno-associated viral (AAV) vector, and a virus-like particle (VLP).

Embodiment 115. The method of embodiment 112, wherein the one or more cells are genetically modified via introduction of a nanoparticle comprising a nucleic acid encoding the chimeric receptor.

Embodiment 116. The method of embodiment 115, wherein the nanoparticle is a lipid nanoparticle (LNP).

Embodiment 117. The method of embodiment 112, wherein the one or more cells are genetically modified via electroporation of a nucleic acid encoding the chimeric receptor.

Embodiment 118. The method of any one of embodiments 112-117, wherein the Tcons are genetically modified from approximately two days to approximately 45 days prior to administration to the human subject.

Embodiment 119. The method of any one of embodiments 112-117, wherein the Tcons are genetically modified approximately 5 days, approximately 10 days, approximately 15 days, approximately 20 days, approximately 25 days, approximately 30 days, approximately 35 days, approximately 40 days, approximately 45 days, approximately 50 days, approximately 55 days, approximately 60 days, approximately 65 days, approximately 70 days, approximately 75 days, approximately 80 days, approximately 85 days, approximately 90 days, approximately 95 days, approximately 100 days, approximately 105 days, approximately 110 days, approximately 115 days, approximately 120 days, approximately 125 days, approximately 130 days, approximately 135 days, approximately 140 days, approximately 145 days, approximately 150 days, approximately 155 days, approximately 160 days, approximately 165 days, approximately 170 days, approximately 175 days, approximately 180 days or more days prior to administration to the human subject.

Embodiment 120. The method of embodiment 118 or embodiment 119, wherein after genetic modification the Tcons are cryogenically preserved until administration to the human subject.

Embodiment 121. The method of any one of embodiments 26-120, wherein the multi-component cellular therapy reduces incidence of transplant related mortality (TRM), as compared to a human subject administered only one or more cells expressing a chimeric receptor.

Embodiment 122. The method of any one of embodiments 26-121, wherein the multi-component cellular therapy reduces incidence and/or severity of graft vs host disease (GVHD), as compared to a human subject administered only one or more cells expressing a chimeric receptor.

Embodiment 123. The method of any one of embodiments 26-122, wherein the multi-component cellular therapy reduces incidence and/or severity of cytokine release syndrome (CRS), as compared to a human subject administered only one or more cells expressing a chimeric receptor.

Embodiment 124. The method of any one of embodiments 26-123, wherein the multi-component cellular therapy reduces incidence and/or severity of immune effector cell-associated neurotoxicity syndrome (ICANS), as compared to a human subject administered only one or more cells expressing a chimeric receptor.

Embodiment 125. A method of treating a human subject having or suspected of having a hematologic malignancy, comprising administering to the human subject a multi-component cellular therapy, wherein the multi-component cellular therapy comprises:
(a) a population of CD34+ hematopoietic stem and progenitor cells (HSPCs), a population of CD4+CD25+CD127dim regulatory T cells (Tregs), a population of conventional CD3+ T cells (Tcons), or any combination thereof; and
(b) a population of tumor-infiltrating lymphocytes (TILs).

Embodiment 126. The method of embodiment 125, wherein the population of Tcons comprises the population of TILs.

Embodiment 127. The method of embodiment 125 or embodiment 126, wherein the multi-component cellular therapy further comprises a population of CD45RA− memory T cells (Tmems).

Embodiment 128. The method of any one of embodiments 1-127, wherein the Tcons are administered approximately 12 hours or more after the HSPCs, after the Tregs, after the Tmems, or after any combination of the HSPCs, Tregs, and Tmems.

Embodiment 129. The method of any one of embodiments 1-128, wherein the Tcons are administered from approximately 24 to approximately 120 hours after the HSPCs, after the Tregs, after the Tmems, or after any combination of the HSPCs, Tregs, and Tmems.

Embodiment 130. The method of any one of embodiments 1-129, wherein the Tcons are administered from approximately 36 to approximately 72 hours after the HSPCs, after the Tregs, after the Tmems, or after any combination of the HSPCs, Tregs, and Tmems.

Embodiment 131. The method of any one of embodiments 1-130, wherein the Tregs, the Tmems, or the Tregs and the Tmems are administered from approximately 5 minutes to approximately 5 hours after administration of the HSPCs.

Embodiment 132. The method of any one of embodiments 1-130, wherein the Tregs and the HSPCs are administered on day 0.

Embodiment 133. The method of embodiment 132, wherein the Tcons are administered on day 2.

Embodiment 134. The method of embodiment 132 or 133, wherein the one or more cells comprising a chimeric receptor are administered on day 2.

Embodiment 135. The method of embodiment 134, wherein the one or more cells comprising a chimeric receptor are administered after the administration of the Tcons.

Embodiment 136. The method of any one of embodiments 1-130, wherein the Tregs and the HSPCs are administered on day 0 and wherein the Tcons and the one or more cells comprising a chimeric receptor are administered on day 2, and wherein the one or more cells comprising a chimeric receptor are administered after the administration of the Tcons.

Embodiment 137. The method of any one of embodiments 1-131, wherein the one or more cells comprising a chimeric receptor are administered approximately 12 hours or more after the HSPCs, after the Tregs, after the Tmems, or after any combination of the HSPCs, Tregs, and Tmems.

Embodiment 138. The method of any one of embodiments 1-137, wherein the one or more cells comprising a chimeric receptor are administered from approximately 24 to approximately 120 hours after the HSPCs, after the Tregs, after the Tmems, or after any combination of the HSPCs, Tregs, and Tmems.

Embodiment 139. The method of any one of embodiments 1-138, wherein the one or more cells comprising a chimeric receptor are administered from approximately 36 to approximately 72 hours after the HSPCs, after the Tregs, after the Tmems, or after any combination of the HSPCs, Tregs, and Tmems.

Embodiment 140. The method of any one of embodiments 1-139, wherein the one or more cells comprising a chimeric receptor are administered from approximately 5 minutes to approximately 5 hours after administration of the Tcons.

Embodiment 141. The method of any one of embodiments 1-140, wherein the method further comprises administering a solution comprising one or more doses of a graft vs host disease (GVHD) prophylactic agent.

Embodiment 142. The method of embodiment 141, wherein the GVHD prophylactic agent is tacrolimus.

Embodiment 143. The method of embodiment 142, wherein the tacrolimus is provided in an amount sufficient to maintain a trough blood level of approximately 5 ng/mL to approximately 15 ng/mL in the human subject.

Embodiment 144. The method of embodiment 142 or embodiment 143, wherein the tacrolimus is provided at a dose that ranges from approximately 0.01 mg per kilogram of body weight of the human subject to approximately 0.50 mg per kilogram of body weight of the human subject twice per day.

Embodiment 145. The method of any one of embodiments 142-144, wherein the tacrolimus is dosed in an amount to maintain or that maintains a target blood level of approximately 1 ng/mL to approximately 10 ng/mL in the human subject for approximately 20 days or more, approximately 25 days or more, approximately 30 days or more, approximately 35 days or more, approximately 40 days or more, approximately 55 days or more, approximately 50 days or more, approximately 55 days or more, approximately 60 days or more, approximately 65 days or more, approximately 770 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days, after administration of the Tcons, after administration of the one or more cells comprising a chimeric receptor, or, after administration of the Tcons and the one or more cells comprising a chimeric receptor.

Embodiment 146. The method of any one of embodiments 142-145, wherein the tacrolimus is initially administered from approximately 12 hours to approximately 24 hours after the Tcons, after the one or more cells comprising a chimeric receptor, or after the Tcons and the one or more cells comprising a chimeric receptor.

Embodiment 147. The method of any one of embodiments 142-146, wherein administration of the tacrolimus is tapered starting at approximately 90 days after initial administration of the tacrolimus.

Embodiment 148. The method of any one of embodiments 1-147, wherein the human subject is administered a dose of HSPCs that ranges from approximately 1.0×105 to approximately 1.0×108 HSPCs per kilogram of body weight of the human subject, or from approximately 5.0×105 to approximately 1.5×1010 HSPCs.

Embodiment 149. The method of any one of embodiments 1-148, wherein the Tregs are FOXP3+.

Embodiment 150. The method of any one of embodiments 1-149, wherein the human subject is administered a dose of Tregs that ranges from approximately 1.0×105 to approximately 2.0×107 Tregs per kilogram of body weight of the human subject, or from approximately 5.0×105 to approximately 3.0×109 Tregs.

Embodiment 151. The method of any one of embodiments 1-150, wherein the human subject is administered a dose of Tmems that ranges from approximately 1.0×105 to approximately 1.0×108 Tmems per kilogram of body weight of the human subject, or from approximately 5.0×105 to approximately 1.5×1010 Tmems.

Embodiment 152. The method of any one of embodiments 1-151, wherein the human subject is administered a dose of Tcons that ranges from approximately 1.0×105 to approximately 4.0×107 Tcons per kilogram of body weight of the human subject, or from approximately 5.0×105 to approximately 6.0×109 Tcons.

Embodiment 153. The method of any one of embodiments 1-152, wherein the one or more cells comprising a chimeric receptor are autologous with respect to the human subject.

Embodiment 154. The method of any one of embodiments 1-152, wherein the one or more cells comprising a chimeric receptor are allogeneic with respect to the human subject.

Embodiment 155. The method of embodiment 154, wherein the one or more cells comprising a chimeric receptor comprise one or more genetic modifications to reduce risk of graft versus host disease (GVHD) and/or to prevent allogeneic rejection with respect to the human subject.

Embodiment 156. The method of embodiment 154, wherein the one or more cells comprising a chimeric receptor have not been modified to reduce risk of graft versus host disease (GVHD) and/or to prevent allogeneic rejection with respect to the human subject.

Embodiment 157. The method of embodiment 156, wherein the one or more cells comprising a chimeric receptor are derived from an allogeneic donor.

Embodiment 158. The method of any one of embodiments 1-157, wherein the HSPCs, the Tregs, Tmems, the Tcons, or any combination thereof are isolated from a donor.

Embodiment 159. The method of embodiment 158, wherein the donor is an allogeneic donor.

Embodiment 160. The method of any one of embodiments 154-159, wherein the HSPCs, the Tregs, Tmems, the Tcons, or any combination thereof, and the one or more cells comprising a chimeric receptor are derived from the same allogeneic donor.

Embodiment 161. The method of embodiment 159 or embodiment 160, wherein the allogeneic donor is unrelated to the human subject.

Embodiment 162. The method of embodiment 159 or embodiment 160, wherein the allogeneic donor is related to the human subject.

Embodiment 163. The method of any one of embodiments 154-162, wherein the allogeneic donor is HLA-matched, relative to the human subject, at one or more alleles selected from the group consisting of: HLA-A, HLA-B, HLA-C, and HLA-DRB1.

Embodiment 164. The method of any one of embodiments 154-163, wherein the allogeneic donor is 8/8 HLA-matched, relative to the human subject, for alleles HLA-A, HLA-B, HLA-C, and HLA-DRB1.

Embodiment 165. The method of any one of embodiments 154-162, wherein the allogeneic donor has at least one HLA mismatch, relative to the human subject.

Embodiment 166. The method of embodiment 165, wherein the at least one HLA mismatch is at an allele selected from the group consisting of: HLA-A, HLA-B, HLA-C, HLA-DRB1, and any combination thereof.

Embodiment 167. The method of any one of embodiments 154-162, wherein the allogeneic donor is an allogeneic haploidentical donor, relative to the human subject.

Embodiment 168. The method of embodiment 167, wherein the allogeneic haploidentical donor is at least 4/8 HLA-mismatched, at least 5/8 HLA-mismatched, or at least 6/8 HLA-mismatched at an allele selected from the group consisting of: HLA-A, HLA-B, HLA-C, HLA-DRB1, and any combination thereof.

Embodiment 169. The method of any one of embodiments 1-168, wherein the method further comprises obtaining one or more cord blood samples from a donor.

Embodiment 170. The method of any one of embodiments 1-168, wherein the method further comprises collecting one or more bone marrow samples from a donor.

Embodiment 171. The method of any one of embodiments 1-168, wherein the method further comprises collecting two or more peripheral blood donations from a donor.

Embodiment 172. The method of embodiment 170 or embodiment 171, wherein at least one of the two or more peripheral blood donations are mobilized peripheral blood donations.

Embodiment 173. The method of any one of embodiments 170-172, wherein at least one of the two or more peripheral blood donations are non-mobilized peripheral blood donations.

Embodiment 174. The method of any one of embodiments 170-173, wherein one or more non-mobilized peripheral blood donations are collected from the donor.

Embodiment 175. The method of embodiment 174, wherein one or more cells obtained from the one or more non-mobilized peripheral blood donations are used to produce the one or more cells comprising a chimeric receptor.

Embodiment 176. The method of any one of embodiments 170-175, wherein one or more mobilized peripheral blood donations are collected from the donor.

Embodiment 177. The method of embodiment 176, wherein two or more mobilized peripheral blood donations are collected from the donor.

Embodiment 178. The method of embodiment 176 or embodiment 177, wherein the HSPCs, the Tregs, Tmems, the Tcons, or any combination thereof are obtained from the mobilized peripheral blood donations.

Embodiment 179. The method of any one of embodiments 172-178, wherein at least one mobilized peripheral blood donation is mobilized by granulocyte colony-stimulating factor (G-CSF), granulocyte macrophage colony-stimulating factor (GM-CSF), mozobil, or any combination thereof.

Embodiment 180. The method of any one of embodiments 172-179, wherein at least one mobilized peripheral blood donation is processed and sorted using one or more immune-separation particles (ISPs) to enrich for HSPCs, Tmems, Tregs, or any combination thereof.

Embodiment 181. The method of embodiment 180, wherein the one or more ISPs comprise affinity reagents, optionally wherein the affinity reagents are immuno-magnetic separation particles, optionally wherein the immuno-magnetic separation particles are antibodies each conjugated to an iron-containing particle.

Embodiment 182. The method of any one of embodiments 1-181, wherein the method further comprises a conditioning regimen, wherein the conditioning regimen is administered before administration of the cellular therapy.

Embodiment 183. The method of embodiment 182, wherein the conditioning regimen is administered from approximately two days to approximately ten days before administration of the multi-component cellular therapy.

Embodiment 184. The method of embodiment 182 or embodiment 183, wherein the conditioning regimen is a total body irradiation-based (TBI-based) regimen or a total marrow and lymphoid irradiation-based (TMLI-based) regimen.

Embodiment 185. The method of embodiment 184, wherein the TBI-based regimen comprises fractionated total body irradiation (fTBI).

Embodiment 186. The method of embodiment 185, wherein the fTBI comprises 2 or more, 3 or more, 4 or more, 5 or more, 6 or more, 7 or more, 8 or more, 9 or more, or 10 or more doses.

Embodiment 187. The method of embodiment 185 or embodiment 186, wherein the fTBI comprises a total dose that ranges from approximately 500 to approximately 1600 cGy.

Embodiment 188. The method of any one of embodiments 185-187, wherein the fTBI comprises a total dose of approximately 1200 cGy, approximately 1210 cGy, approximately 1220, approximately 1240 cGy, approximately 1250 cGy approximately 1260 cGy, approximately 1270 cGy, approximately 1280 cGy, approximately 1290 cGy, approximately 1300 cGy, approximately 1310 cGy, approximately 1320, approximately 1340 cGy, approximately 1350 cGy approximately 1360 cGy, approximately 1370 cGy, approximately 1380 cGy, approximately 1390 cGy, approximately 1400 cGy, approximately 1410 cGy, approximately 1420, approximately 1440 cGy, approximately 1450 cGy approximately 1460 cGy, approximately 1470 cGy, approximately 1480 cGy, approximately 1490 cGy, or approximately 1500 cGy.

Embodiment 189. The method of any one of embodiments 185-188, wherein the fTBI-based regimen comprises 4 doses of TBI.

Embodiment 190. The method of any one of embodiments 184-189, wherein the TBI-based or TMLI-based regimen further comprises one or more doses of a conditioning reagent.

Embodiment 191. The method of embodiment 190, wherein the conditioning reagent comprises one or more conditioning reagents selected from the group consisting of: cyclophosphamide, etoposide, thiotepa, busulfan, melphalan, fludarabine, and any combination thereof.

Embodiment 192. The method of embodiment 190 or embodiment 191, wherein the TBI-based or TMLI-based regimen further comprises one or more doses of cyclophosphamide.

Embodiment 193. The method of embodiment 192, wherein each of the one or more doses of cyclophosphamide comprise from approximately 10 to approximately 100 mg of cyclophosphamide per kilogram of body weight of the human subject.

Embodiment 194. The method of embodiment 192 or embodiment 193, wherein the one or more doses of cyclophosphamide comprise approximately 10 mg of cyclophosphamide per kilogram of body weight of the human subject, approximately 15 mg of cyclophosphamide per kilogram of body weight of the human subject, approximately 20 mg of cyclophosphamide per kilogram of body weight of the human subject, approximately 25 mg of cyclophosphamide per kilogram of body weight of the human subject, approximately 30 mg of cyclophosphamide per kilogram of body weight of the human subject, approximately 35 mg of cyclophosphamide per kilogram of body weight of the human subject, approximately 40 mg of cyclophosphamide per kilogram of body weight of the human subject, approximately 45 mg of cyclophosphamide per kilogram of body weight of the human subject, approximately 50 mg of cyclophosphamide per kilogram of body weight of the human subject, approximately 55 mg of cyclophosphamide per kilogram of body weight of the human subject, or approximately 60 mg of cyclophosphamide per kilogram of body weight of the human subject.

Embodiment 195. The method of any one of embodiments 185-194, wherein the fTBI-based regimen further comprises two doses of cyclophosphamide.

Embodiment 196. The method of any one of embodiments 185-195, wherein a first dose of TBI or TMLI is administered approximately 8 days prior to administration of the HSPCs, prior to administration of the Tregs, prior to administration of the Tmems, or prior to administration of any combination of HSPCs, Tregs, and Tmems.

Embodiment 197. The method of any one of embodiments 185-196, wherein a second dose of TBI is administered approximately 7 days prior to administration of the HSPCs, prior to administration of the Tregs, prior to administration of the Tmems, or prior to administration of any combination of HSPCs, Tregs, and Tmems.

Embodiment 198. The method of any one of embodiments 185-197, wherein a third dose of TBI is administered approximately 6 days prior to administration of the HSPCs, prior to administration of the Tregs, prior to administration of the Tmems, or prior to administration of any combination of HSPCs, Tregs, and Tmems.

Embodiment 199. The method of any one of embodiments 185-198, wherein a fourth dose of TBI is administered approximately 5 days prior to administration of the HSPCs, prior to administration of the Tregs, prior to administration of the Tmems, or prior to administration of any combination of HSPCs, Tregs, and Tmems.

Embodiment 200. The method of any one of embodiments 185-199, wherein a first dose of cyclophosphamide is administered approximately 4 days prior to administration of the HSPCs, prior to administration of the Tregs, prior to administration of the Tmems, or prior to administration of any combination of HSPCs, Tregs, and Tmems.

Embodiment 201. The method of any one of embodiments 185-200, wherein a second dose of cyclophosphamide is administered approximately 4 days prior to administration of the HSPCs, prior to administration of the Tregs, prior to administration of the Tmems, or prior to administration of any combination of HSPCs, Tregs, and Tmems.

Embodiment 202. The method of embodiment 182 or embodiment 183, wherein the conditioning regimen is a myeloablative conditioning regimen or a reduced-intensity conditioning (RIC) regimen.

Embodiment 203. The method of embodiment 202, wherein the myeloablative conditioning regimen or RIC regimen comprises one or more conditioning reagents.

Embodiment 204. The method of embodiment 203, wherein the one or more conditioning reagents are selected from the group consisting of: thiotepa, busulfan, melphalan, fludarabine, cyclophosphamide, anti-thymocyte globulin (ATG), and any combination thereof.

Embodiment 205. The method of any one of embodiments 202-204, wherein the myeloablative conditioning regimen comprises three or more conditioning reagents, wherein at least one conditioning reagent comprises thiotepa.

Embodiment 206. The method of any one of embodiments 202-205, wherein the myeloablative conditioning regimen comprises one or more doses of busulfan, one or more doses of fludarabine, and one or more doses of thiotepa.

Embodiment 207. The method of embodiment 206, wherein each of the one or more doses of busulfan comprise from approximately 2 to approximately 11 mg of busulfan per kilogram of body weight of the human subject.

Embodiment 208. The method of embodiment 206 or embodiment 207, wherein each of the one or more doses of busulfan comprise approximately 2.0 mg of busulfan per kilogram of body weight of the human subject, approximately 2.1 mg of busulfan per kilogram of body weight of the human subject, approximately 2.2 mg of busulfan per kilogram of body weight of the human subject, approximately 2.3 mg of busulfan per kilogram of body weight of the human subject, approximately 2.4 mg of busulfan per kilogram of body weight of the human subject, approximately 2.5 mg of busulfan per kilogram of body weight of the human subject, approximately 2.6 mg of busulfan per kilogram of body weight of the human subject, approximately 2.7 mg of busulfan per kilogram of body weight of the human subject, approximately 2.8 mg of busulfan per kilogram of body weight of the human subject, approximately 2.9 mg of busulfan per kilogram of body weight of the human subject, approximately 3.0 mg of busulfan per kilogram of body weight of the human subject, approximately 3.1 mg of busulfan per kilogram of body weight of the human subject, approximately 3.2 mg of busulfan per kilogram of body weight of the human subject, approximately 3.3 mg of busulfan per kilogram of body weight of the human subject, approximately 3.4 mg of busulfan per kilogram of body weight of the human subject, approximately 3.5 mg of busulfan per kilogram of body weight of the human subject, approximately 3.6 mg of busulfan per kilogram of body weight of the human subject, approximately 3.7 mg of busulfan per kilogram of body weight of the human subject, approximately 3.8 mg of busulfan per kilogram of body weight of the human subject, approximately 3.9 mg of busulfan per kilogram of body weight of the human subject, approximately 4.0 mg of busulfan per kilogram of body weight of the human subject, approximately 4.1 mg of busulfan per kilogram of body weight of the human subject, approximately 4.2 mg of busulfan per kilogram of body weight of the human subject, approximately 4.3 mg of busulfan per kilogram of body weight of the human subject, approximately 4.4 mg of busulfan per kilogram of body weight of the human subject, approximately 4.5 mg of busulfan per kilogram of body weight of the human subject, approximately 4.6 mg of busulfan per kilogram of body weight of the human subject, approximately 4.7 mg of busulfan per kilogram of body weight of the human subject, approximately 4.8 mg of busulfan per kilogram of body weight of the human subject, approximately 4.9 mg of busulfan per kilogram of body weight of the human subject, approximately 5.0 mg of busulfan per kilogram of body weight of the human subject, approximately 5.1 mg of busulfan per kilogram of body weight of the human subject, approximately 5.2 mg of busulfan per kilogram of body weight of the human subject, approximately 5.3 mg of busulfan per kilogram of body weight of the human subject, approximately 5.4 mg of busulfan per kilogram of body weight of the human subject, approximately 5.5 mg of busulfan per kilogram of body weight of the human subject, approximately 5.6 mg of busulfan per kilogram of body weight of the human subject, approximately 5.7 mg of busulfan per kilogram of body weight of the human subject, approximately 5.8 mg of busulfan per kilogram of body weight of the human subject, approximately 5.9 mg of busulfan per kilogram of body weight of the human subject, or approximately 6.0 mg of busulfan per kilogram of body weight of the human subject.

Embodiment 209. The method of embodiment 83 or embodiment 208, wherein the myeloablative conditioning regimen comprises three doses of busulfan.

Embodiment 210. The method of any one of embodiments 206-209, wherein each of the one or more doses of fludarabine comprise from approximately 30 to approximately 200 mg of fludarabine per meter2 body surface area.

Embodiment 211. The method of any one of embodiments 206-210, wherein each of the one or more doses of fludarabine comprise 40 mg of fludarabine per meter2 body surface area, 41 mg of fludarabine per meter2 body surface area, 42 mg of fludarabine per meter2 body surface area, 43 mg of fludarabine per meter2 body surface area, 44 mg of fludarabine per meter2 body surface area, 44 mg of fludarabine per meter2 body surface area, 46 mg of fludarabine per meter2 body surface area, 47 mg of fludarabine per meter2 body surface area, 48 mg of fludarabine per meter2 body surface area, 49 mg of fludarabine per meter2 body surface area, approximately 50 mg of fludarabine per meter2 body surface area, 51 mg of fludarabine per meter2 body surface area, 52 mg of fludarabine per meter2 body surface area, 53 mg of fludarabine per meter2 body surface area, 54 mg of fludarabine per meter2 body surface area, 55 mg of fludarabine per meter2 body surface area, 56 mg of fludarabine per meter2 body surface area, 57 mg of fludarabine per meter2 body surface area, 58 mg of fludarabine per meter2 body surface area, 59 mg of fludarabine per meter2 body surface area, approximately 60 mg of fludarabine per meter2 body surface area, 61 mg of fludarabine per meter2 body surface area, 62 mg of fludarabine per meter2 body surface area, 63 mg of fludarabine per meter2 body surface area, 64 mg of fludarabine per meter2 body surface area, 65 mg of fludarabine per meter2 body surface area, 66 mg of fludarabine per meter2 body surface area, 67 mg of fludarabine per meter2 body surface area, 68 mg of fludarabine per meter2 body surface area, 69 mg of fludarabine per meter2 body surface area, approximately 70 mg of fludarabine per meter2 body surface area, 71 mg of fludarabine per meter2 body surface area, 72 mg of fludarabine per meter2 body surface area, 73 mg of fludarabine per meter2 body surface area, 74 mg of fludarabine per meter2 body surface area, or 75 mg of fludarabine per meter2 body surface area.

Embodiment 212. The method of embodiment 210 or embodiment 211, wherein the myeloablative conditioning regimen comprises three doses of fludarabine.

Embodiment 213. The method of any one of embodiments 206-211, wherein each of the one or more doses of thiotepa comprise from approximately 3 to approximately 12 mg of thiotepa per kilogram of body weight of the human subject.

Embodiment 214. The method of any one of embodiments 206-210, wherein each of the one or more doses of thiotepa comprise approximately 3.0 mg of thiotepa per kilogram of body weight of the human subject, 3.1 mg of thiotepa per kilogram of body weight of the human subject, 3.2 mg of thiotepa per kilogram of body weight of the human subject, 3.3 mg of thiotepa per kilogram of body weight of the human subject, 3.4 mg of thiotepa per kilogram of body weight of the human subject, 3.5 mg of thiotepa per kilogram of body weight of the human subject, 3.6 mg of thiotepa per kilogram of body weight of the human subject, 3.7 mg of thiotepa per kilogram of body weight of the human subject, 3.8 mg of thiotepa per kilogram of body weight of the human subject, 3.9 mg of thiotepa per kilogram of body weight of the human subject, approximately 4.0 mg of thiotepa per kilogram of body weight of the human subject, 4.1 mg of thiotepa per kilogram of body weight of the human subject, 4.2 mg of thiotepa per kilogram of body weight of the human subject, 4.3 mg of thiotepa per kilogram of body weight of the human subject, 4.4 mg of thiotepa per kilogram of body weight of the human subject, 4.5 mg of thiotepa per kilogram of body weight of the human subject, 4.6 mg of thiotepa per kilogram of body weight of the human subject, 4.7 mg of thiotepa per kilogram of body weight of the human subject, 4.8 mg of thiotepa per kilogram of body weight of the human subject, 4.9 mg of thiotepa per kilogram of body weight of the human subject, approximately 5.0 mg of thiotepa per kilogram of body weight of the human subject, 5.1 mg of thiotepa per kilogram of body weight of the human subject, 5.2 mg of thiotepa per kilogram of body weight of the human subject, 5.3 mg of thiotepa per kilogram of body weight of the human subject, 5.4 mg of thiotepa per kilogram of body weight of the human subject, 5.5 mg of thiotepa per kilogram of body weight of the human subject, 5.6 mg of thiotepa per kilogram of body weight of the human subject, 5.7 mg of thiotepa per kilogram of body weight of the human subject, 5.8 mg of thiotepa per kilogram of body weight of the human subject, 5.9 mg of thiotepa per kilogram of body weight of the human subject, approximately 6.0 mg of thiotepa per kilogram of body weight of the human subject, 6.1 mg of thiotepa per kilogram of body weight of the human subject, 6.2 mg of thiotepa per kilogram of body weight of the human subject, 6.3 mg of thiotepa per kilogram of body weight of the human subject, 6.4 mg of thiotepa per kilogram of body weight of the human subject, 6.5 mg of thiotepa per kilogram of body weight of the human subject, 6.6 mg of thiotepa per kilogram of body weight of the human subject, 6.7 mg of thiotepa per kilogram of body weight of the human subject, 6.8 mg of thiotepa per kilogram of body weight of the human subject, 6.9 mg of thiotepa per kilogram of body weight of the human subject, or 7.0 mg of thiotepa per kilogram of body weight of the human subject.

Embodiment 215. The method of embodiment 213 or embodiment 214, wherein the myeloablative conditioning regimen comprises two doses of thiotepa.

Embodiment 216. The method of any one of embodiments 206-215, wherein a first dose of busulfan and a first dose of fludarabine are administered approximately 6 days prior to administration of the HSPCs, prior to administration of the Tregs, prior to administration of the Tmems, or prior to administration of any combination of HSPCs, Tregs, and Tmems.

Embodiment 217. The method of any one of embodiments 206-216, wherein a second dose of busulfan and a second dose of fludarabine are administered approximately 5 days prior to administration of the HSPCs, prior to administration of the Tregs, prior to administration of the Tmems, or prior to administration of any combination of HSPCs, Tregs, and Tmems.

Embodiment 218. The method of any one of embodiments 206-217, wherein a third dose of busulfan and a third dose of fludarabine are administered approximately 4 days prior to administration of the HSPCs, prior to administration of the Tregs, prior to administration of the Tmems, or prior to administration of any combination of HSPCs, Tregs, and Tmems.

Embodiment 219. The method of any one of embodiments 206-218, wherein a first dose of thiotepa is administered approximately 3 days prior to administration of the HSPCs, prior to administration of the Tregs, prior to administration of the Tmems, or prior to administration of any combination of HSPCs, Tregs, and Tmems.

Embodiment 220. The method of any one of embodiments 206-219, wherein a second dose of thiotepa is administered approximately 2 days prior to administration of the HSPCs, prior to administration of the Tregs, prior to administration of the Tmems, or prior to administration of any combination of HSPCs, Tregs, and Tmems.

Embodiment 221. The method of any one of embodiments 1-220, wherein the chimeric receptor comprises one or more extracellular antigen-binding domains that bind to one or more antigens selected from the group consisting of: BCMA, PSMA, CD8, CD10, CD19, CD20, CD21, CD22, CD25, CD30, CD33, CD34, CD37, CD44v6, CD45, CD52, CSF1R (CD115), c-Kit (CD117), CD123, CD133, Fms-like tyrosine kinase 3 (FLT-3, CD135), PDGFR-alpha (CD140a), PDGFR-beta (CD140b), CD186, CD195, VEGFR3 (FLT4, CD309), GPRC5D, TMEFF2, ROR1, B7-H6, B7-H3, HM1.24, SLAMF7, chondroitin sulfate proteoglycan 4 (CSPG4, melanoma-associated chondroitin sulfate proteoglycan), epidermal growth factor receptor (EGFR), Her2, Her3, IGFR, IL3R, fibroblast activating protein (FAP), CDCP1, Derlin1, Tenascin, frizzled 1-10, VEGFR2 (KDR/FLK1), endoglin, CLEC14, Tem1-8, Tie2, A33, CAMPATH-1 (CDw52), CEA, Carbonic anhydrase IX (MN/CA IX), de2-7, EGFRvIII, EpCAM, Ep-CAM, folate-binding protein, G250, HLA-DR, IGFR, IL-2 receptor, IL3R, MCSP (melanoma-associated cell surface chondroitin sulphate proteoglycane), Muc-1, prostate stem cell antigen (PSCA), prostate specific antigen (PSA), hK2, TAG-72, KI2L4, NKG2E, NKG2D, NKG2F, BTNL3, BTNL8, PD-1, NKG2, a tumor cell neoantigen, and any combination thereof.

Embodiment 222. The method of embodiment 221, wherein the chimeric receptor is a chimeric antigen receptor (CAR) or T cell receptor (TCR).

Embodiment 223. The method of embodiment 222, wherein the CAR further comprises a transmembrane domain and one or more intracellular signaling domains.

Embodiment 224. The method of embodiment 223, wherein the transmembrane domain is selected from the group consisting of: a CD8 transmembrane domain, a CD28 transmembrane domain a CD3zeta-chain transmembrane domain, a CD4 transmembrane domain, a 4-IBB transmembrane domain, an 0X40 transmembrane domain, an ICOS transmembrane domain, a CTLA-4 transmembrane domain, aPD-1 transmembrane domain, a LAG-3 transmembrane domain, a 2B4 transmembrane domain, a BTLA transmembrane domain, an 0X40 transmembrane domain, a DAP 10 transmembrane domain, a DAP 12 transmembrane domain, a CD16a transmembrane domain, a DNAM-1 transmembrane domain, a KIR2DSl transmembrane domain, aKIR3DSl transmembrane domain, an NKp44 transmembrane domain, an NKp46 transmembrane domain, an FceRlg transmembrane domain, and an NKG2D transmembrane domain.

Embodiment 225. The method of embodiment 223 or embodiment 224, wherein the one or more intracellular signaling domains are each selected from the group consisting of: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CDS intracellular signaling domain, an 0X40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP 10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceRlg intracellular signaling domain, a NKG2D intracellular signaling domain, and an EAT-2 intracellular signaling domain.

Embodiment 226. The method of any one of embodiments 222-225, wherein the CAR is a bispecific CAR that comprises two or more extracellular antigen-binding domains.

Embodiment 227. The method of any one of embodiments 222-226, wherein the CAR comprises a first antigen-binding domain specific for CD22 and a second antigen-binding domain specific for CD19.

Embodiment 228. The method of embodiment 227, wherein the first antigen-binding domain comprises a heavy chain variable (VH) region and a light chain variable (VL) region,
wherein the VH comprises: a heavy chain complementarity determining region 1 (CDR-H1) having the amino acid sequence of GDSVSSNSAA (SEQ ID NO: 1), a heavy chain complementarity determining region 2 (CDR-H2) having the amino acid sequence of TYYRSKWYN (SEQ ID NO: 2), and a heavy chain complementarity determining region 3 (CDR-H3) having the amino acid sequence of AREVTGDLEDAFDI (SEQ ID NO: 3), and
wherein the VL comprises: a light chain complementarity determining region 1 (CDR-L1) having the amino acid sequence of QTIWSY (SEQ ID NO: 4), a light chain complementarity determining region 2 (CDR-L2) having the amino acid sequence of AAS (SEQ ID NO: 5), and a light chain complementarity determining region 3 (CDR-L3) having the amino acid sequence of QQSYSIPQT (SEQ ID NO: 6).

Embodiment 229. The method of embodiment 227 or embodiment 228, wherein the second antigen-binding domain comprises a heavy chain variable (VH) region and a light chain variable (VL) region, wherein the VH comprises: a heavy chain complementarity determining region 1 (CDR-H1) having the amino acid sequence of GVSLPDYG (SEQ ID NO: 7), a heavy chain complementarity determining region 2 (CDR-H2) having the amino acid sequence of IWGSETT (SEQ ID NO: 8), and a heavy chain complementarity determining region 3 (CDR-H3) having the amino acid sequence of AKHYYYGGSYAMDY (SEQ ID NO: 9), and
wherein the VL comprises: a light chain complementarity determining region 1 (CDR-L1) having the amino acid sequence of QDISKY (SEQ ID NO: 10), a light chain complementarity determining region 2 (CDR-L2) having the amino acid sequence of HTS (SEQ ID NO: 11), and a light chain complementarity determining region 3 (CDR-L3) having the amino acid sequence of QQGNTLPYT (SEQ ID NO: 12).

Embodiment 230. The method of any one of embodiments 227-229 wherein the first antigen-binding domain comprises a VH having amino acid sequence of

(SEQ ID NO: 13)
QVQLQQSGPGLVKPSQTLSLTCAISGDSVSSNSAAWNWIRQSPS
RGLEWLGRTYYRSKWYNDYAVSVKSRITINPDTSKNQFSLQLNS
VTPEDTAVYYCAREVTGDLEDAFDIWGQGTMVTVSS.

Embodiment 231. The method of any one of embodiments 227-230, wherein the first antigen-binding domain comprises a VL having amino acid sequence of

(SEQ ID NO: 14)
DIQMTQSPSSLSASVGDRVTITCRASQTIWSYLNWYQQRPGKAPNLLI
YAASSLQSGVPSRFSGRGSGTDFTLTISSLQAEDFATYYCQQSYSIPQ
TFGQGTKLEIK.

Embodiment 232. The method of any one of embodiments 227-231, wherein the second antigen-binding domain comprises a VH having amino acid sequence of

(SEQ ID NO: 15)
EVKLQESGPGLVAPSQSLSVTCTVSGVSLPDYGVSWIRQPPRKGLEWL
GVIWGSETTYYNSALKSRLTIIKDNSKSQVFLKMNSLQTDDTAIYYCA
KHYYYGGSYAMDYWGQGTSVTVSS.

Embodiment 233. The method of any one of embodiments 227-232, wherein the second antigen-binding domain comprises a VL having the amino acid sequence of

(SEQ ID NO: 16)
DIQMTQTTSSLSASLGDRVTISCRASQDISKYLNWYQQKPDGTVKLLI
YHTSRLHSGVPSRFSGSGSGTDYSLTISNLEQEDIATYFCQQGNTLPY
TFGGGTKLEIT.

Embodiment 234. The method of any one of embodiments 227-233, wherein the first antigen-binding domain comprises a CD22 scFv having the amino acid sequence of:

(a)
(SEQ ID NO: 20)
QVQLQQSGPGLVKPSQTLSLTCAISGDSVSSNSAAWNWIRQSPSRGLE
WLGRTYYRSKWYNDYAVSVKSRITINPDTSKNQFSLQLNSVTPEDTAV
YYCAREVTGDLEDAFDIWGQGTMVTVSSGSTSGSGKPGSGEGSTKGDI
QMTQSPSSLSASVGDRVTITCRASQTIWSYLNWYQQRPGKAPNLLIYA
ASSLQSGVPSRFSGRGSGTDFTLTISSLQAEDFATYYCQQSYSIPQTF
GQGTKLEIK;
or
(b)
(SEQ ID NO: 21)
QVQLQQSGPGLVKPSQTLSLTCAISGDSVSSNSAAWNWIRQSPSRGLE
WLGRTYYRSKWYNDYAVSVKSRITINPDTSKNQFSLQLNSVTPEDTAV
YYCAREVTGDLEDAFDIWGQGTMVTVSSGGGGSDIQMTQSPSSLSAS
VGDRVTITCRASQTIWSYLNWYQQRPGKAPNLLIYAASSLQSGVPSRF
SGRGSGTDFTLTISSLQAEDFATYYCQQSYSIPQTFGQGTKLEIK.

Embodiment 235. The method of any one of embodiment 227-234, wherein the second antigen-binding domain comprises a CD19 scFv having the amino acid sequence of:

(a)
(SEQ ID NO: 22)
DIQMTQTTSSLSASLGDRVTISCRASQDISKYLNWYQQKPDGTVKLLI
YHTSRLHSGVPSRFSGSGSGTDYSLTISNLEQEDIATYFCQQGNTLPY
TFGGGTKLEITGSTSGSGKPGSGEGSTKGEVKLQESGPGLVAPSQSLS
VTCTVSGVSLPDYGVSWIRQPPRKGLEWLGVIWGSETTYYNSALKSRL
TIIKDNSKSQVFLKMNSLQTDDTAIYYCAKHYYYGGSYAMDYWGQGTS
VTVSS;
or
(b)
(SEQ ID NO: 41)
DIQMTQTTSSLSASLGDRVTISCRASQDISKYLNWYQQKPDGTVKLLI
YHTSRLHSGVPSRFSGSGSGTDYSLTISNLEQEDIATYFCQQGNTLPY
TFGGGTKLEITGGGGSEVKLQESGPGLVAPSQSLSVTCTVSGVSLPDY
GVSWIRQPPRKGLEWLGVIWGSETTYYNSALKSRLTIIKDNSKSQVFL
KMNSLQTDDTAIYYCAKHYYYGGSYAMDYWGQGTSVTVSS.

Embodiment 236. The method of any one of embodiments 227-235, wherein the CAR comprises the amino acid sequence of:

(a)
(SEQ ID NO: 23)
DIQMTQTTSSLSASLGDRVTISCRASQDISKYLNWYQQKPDGTVKLLIYHTSRLHSGVPSRFSG
SGSGTDYSLTISNLEQEDIATYFCQQGNTLPYTFGGGTKLEITGGGGSQVQLQQSGPGLVKPSQTL
SLTCAISGDSVSSNSAAWNWIRQSPSRGLEWLGRTYYRSKWYNDYAVSVKSRITINPDTSKNQFS
LQLNSVTPEDTAVYYCAREVTGDLEDAFDIWGQGTMVTVSSGSTSGSGKPGSGEGSTKGDIQMT
QSPSSLSASVGDRVTITCRASQTIWSYLNWYQQRPGKAPNLLIYAASSLQSGVPSRFSGRGSGTDF
TLTISSLQAEDFATYYCQQSYSIPQTFGQGTKLEIKGGGGSEVKLQESGPGLVAPSQSLSVTCTVS
GVSLPDYGVSWIRQPPRKGLEWLGVIWGSETTYYNSALKSRLTIIKDNSKSQVFLKMNSLQTDDT
AIYYCAKHYYYGGSYAMDYWGQGTSVTVSSS;
(b)
(SEQ ID NO: 24)
QVQLQQSGPGLVKPSQTLSLTCAISGDSVSSNSAAWNWIRQSPSRGLEWLGRTYYRSKWYND
YAVSVKSRITINPDTSKNQFSLQLNSVTPEDTAVYYCAREVTGDLEDAFDIWGQGTMVTVSSGG
GGSDIQMTQSPSSLSASVGDRVTITCRASQTIWSYLNWYQQRPGKAPNLLIYAASSLQSGVPSRFS
GRGSGTDFTLTISSLQAEDFATYYCQQSYSIPQTFGQGTKLEIKGGGGSGGGGSGGGGSGGGGSG
GGGSDIQMTQTTSSLSASLGDRVTISCRASQDISKYLNWYQQKPDGTVKLLIYHTSRLHSGVPSR
FSGSGSGTDYSLTISNLEQEDIATYFCQQGNTLPYTFGGGTKLEITGSTSGSGKPGSGEGSTKGEV
KLQESGPGLVAPSQSLSVTCTVSGVSLPDYGVSWIRQPPRKGLEWLGVIWGSETTYYNSALKSRL
TIIKDNSKSQVFLKMNSLQTDDTAIYYCAKHYYYGGSYAMDYWGQGTSVTVSS
(c)
(SEQ ID NO: 29)
DIQMTQTTSSLSASLGDRVTISCRASQDISKYLNWYQQKPDGTVKLLIYHTSRLHSGVPSRFSG
SGSGTDYSLTISNLEQEDIATYFCQQGNTLPYTFGGGTKLEITGGGGSQVQLQQSGPGLVKPSQTL
SLTCAISGDSVSSNSAAWNWIRQSPSRGLEWLGRTYYRSKWYNDYAVSVKSRITINPDTSKNQFS
LQLNSVTPEDTAVYYCAREVTGDLEDAFDIWGQGTMVTVSSGSTSGSGKPGSGEGSTKGDIQMT
QSPSSLSASVGDRVTITCRASQTIWSYLNWYQQRPGKAPNLLIYAASSLQSGVPSRFSGRGSGTDF
TLTISSLQAEDFATYYCQQSYSIPQTFGQGTKLEIKGGGGSEVKLQESGPGLVAPSQSLSVTCTVS
GVSLPDYGVSWIRQPPRKGLEWLGVIWGSETTYYNSALKSRLTIIKDNSKSQVFLKMNSLQTDDT
AIYYCAKHYYYGGSYAMDYWGQGTSVTVSSSGTTTPAPRPPTPAPTIASQPLSLRPEACRPAAGG
AVHTRGLDFACDIYIWAPLAGTCGVLLLSLVITLYCKRGRKKLLYIFKQPFMRPVQTTQEEDGCS
CRFPEEEEGGCELRVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDTYDALHMQALPPR;
or
(d)
(SEQ ID NO: 30)
QVQLQQSGPGLVKPSQTLSLTCAISGDSVSSNSAAWNWIRQSPSRGLEWLGRTYYRSKWYND
YAVSVKSRITINPDTSKNQFSLQLNSVTPEDTAVYYCAREVTGDLEDAFDIWGQGTMVTVSSGG
GGSDIQMTQSPSSLSASVGDRVTITCRASQTIWSYLNWYQQRPGKAPNLLIYAASSLQSGVPSRFS
GRGSGTDFTLTISSLQAEDFATYYCQQSYSIPQTFGQGTKLEIKGGGGSGGGGSGGGGSGGGGSG
GGGSDIQMTQTTSSLSASLGDRVTISCRASQDISKYLNWYQQKPDGTVKLLIYHTSRLHSGVPSR
FSGSGSGTDYSLTISNLEQEDIATYFCQQGNTLPYTFGGGTKLEITGSTSGSGKPGSGEGSTKGEV
KLQESGPGLVAPSQSLSVTCTVSGVSLPDYGVSWIRQPPRKGLEWLGVIWGSETTYYNSALKSRL
TIIKDNSKSQVFLKMNSLQTDDTAIYYCAKHYYYGGSYAMDYWGQGTSVTVSSTTTPAPRPPTP
APTIASQPLSLRPEACRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYKQGQNQLYNELNLGRR
EEYDVLDKRRGRDPEMGGKPRRKNPQEGLYNELQKDKMAEAYSEIGMKGERRRGKGHDGLYQ
GLSTATKDTYDALHMQALPPR.

Embodiment 237. The method of any one of embodiments 222-225, wherein the CAR comprises an antigen-binding domain specific for B-cell maturation antigen (BCMA).

Embodiment 238. The method of embodiment 237, wherein the antigen-binding domain comprises a heavy chain variable (VH) region comprising a heavy chain complementarity determining region 1 (CDR-H1) a complementarity determining region 2 (CDR-H2), and a complementarity determining region 3 (CDR-H3),
wherein the VH comprises CDR-H1, CDR-H2, and CDR-H3 amino acid sequences selected from the group consisting of:

a)
(SEQ ID NO: 42)
GFTFTNHA,
(SEQ ID NO: 43)
ISGNGRTT,
and
(SEQ ID NO: 44)
AKDGGETLVDS;
b)
(SEQ ID NO: 45)
GFTFSSHA;
(SEQ ID NO: 46)
ISGSGDFT,
and
(SEQ ID NO: 47)
AKDEDGGSLLGY;
c)
(SEQ ID NO: 48)
GFTFSSYA;
(SEQ ID NO: 49)
ISGSGDYI,
and
(SEQ ID NO: 50)
AKEGTGANSSLADY;
and
d)
(SEQ ID NO: 51)
GFTFSSHA,
(SEQ ID NO: 52)
ISGSGDYT,
and
(SEQ ID NO: 53)
AKDEDGGSLLGH.

Embodiment 239. The method of embodiment 237 or embodiment 238, wherein the antigen-binding domain comprises a VH having amino acid sequence selected from the group consisting of:

a)
(SEQ ID NO: 54)
QVQLVESGGGLVQPGGSLRLSCAASGFTFTNHAMSWVRQAPGKGLELVS
SISGNGRTTYYADSVKGRFTISRDISKNTLDLQMNSLRAEDTAVYYCAK
DGGETLVDSRGQGTLVTVSS;
b)
(SEQ ID NO: 55)
QVQLVESGGGLVQPGGSLRLSCAASGFTFSSHAMTWVRQAPGKGLEWVA
AISGSGDFTHYADSVKGRFTISRDNSKNTVSLQMNNLRAEDTAVYYCAK
DEDGGSLLGYRGQGTLVTVSS;
c)
(SEQ ID NO: 56)
EVQLLESGGGLVQPGGSLRLSCAASGFTFSSYAMSWVRQAPGKGLEWVS
SISGSGDYIYYADSVKGRFTISRDISKNTLYLQMNSLRAEDTAVYYCAK
EGTGANSSLADYRGQGTLVTVSS;
and
d)
(SEQ ID NO: 57)
EVQLLESGGGLIQPGGSLRLSCAASGFTFSSHAMTWVRQAPGKGLEWVS
AISGSGDYTHYADSVKGRFTISRDNSKNTVYLQMNSLRAEDSAVYYCAK
DEDGGSLLGHRGQGTLVTVSS.

Embodiment 240. The method of embodiment 237, wherein the antigen-binding domain comprises a heavy chain variable (VH) region and a light chain variable (VL) region,
wherein the VH comprises an amino acid sequence selected from the group consisting of:

a)
(SEQ ID NO: 58)
QIQLVQSGPELKKPGETVKISCKASGYTFRHYSMNWVKQAPGKGLKWMG
RINTESGVPIYADDFKGRFAFSVETSASTAYLVINNLKDEDTASYFCSN
DYLYSLDFWGQGTALTVSS;
and
b)
(SEQ ID NO: 60)
QIQLVQSGPELKKPGETVKISCKASGYTFTDYSINWVKRAPGKGLKWMG
WINTETREPAYAYDFRGRFAFSLETSASTAYLQINNLKYEDTATYFCAL
DYSYAMDYWGQGTSVTVSS,

and
wherein the VL comprises an amino acid sequence selected from the group consisting of:

a)
(SEQ ID NO: 59)
DIVLTQSPPSLAMSLGKRATISCRASESVTILGSHLIYWYQQKPGQPPT
LLIQLASNVQTGVPARFSGSGSRTDFTLTIDPVEEDDVAVYYCLQSRTI
PRTFGGGTKLEIK;
b)
(SEQ ID NO: 61)
DIVLTQSPPSLAMSLGKRATISCRASESVTILGSHLIHWYQQKPGQPP
TLLIQLASNVQTGVPARFSGSGSRTDFTLTIDPVEEDDVAVYYCLQSRT
IPRTFGGGTKLEIK;
c)
(SEQ ID NO: 62)
DIVLTQSPASLAVSLGQRATISCRASESVDNYGFSFMHWYQQKPGQPPK
LLIYRASNLESGIPARFSGSGSRTDFALTINPVETDDVATYYCQQSNKD
PRTFGGGTKLEIK;
and
d)
(SEQ ID NO: 63)
DIVLTQSPASLAVSLGQRATISCRASESVDNYGFSFMHWYQQKPGQPPK
LLIYRASNLESGIPARFSGSGSRTDFALTINPVETDDVATYYCQQSNKD
PRTFGGGTKLEIK.

Embodiment 241. The method of embodiment 240, wherein the antigen-binding domain comprises a BCMA scFv having an amino acid sequence selected from the group consisting of:

a)
(SEQ ID NO: 64)
DIVLTQSPPSLAMSLGKRATISCRASESVTILGSHLIYWYQQKPGQPPTLLIQLASNVQTGVPAR
FSGSGSRTDFTLTIDPVEEDDVAVYYCLQSRTIPRTFGGGTKLEIKGSTSGSGKPGSGEGSTKGQIQ
LVQSGPELKKPGETVKISCKASGYTFRHYSMNWVKQAPGKGLKWMGRINTESGVPIYADDFKG
RFAFSVETSASTAYLVINNLKDEDTASYFCSNDYLYSLDFWGQGTALTVSS;
b)
(SEQ ID NO: 65)
DIVLTQSPPSLAMSLGKRATISCRASESVTILGSHLIHWYQQKPGQPPTLLIQLASNVQTGVPAR
FSGSGSRTDFTLTIDPVEEDDVAVYYCLQSRTIPRTFGGGTKLEIKGSTSGSGKPGSGEGSTKGQIQ
LVQSGPELKKPGETVKISCKASGYTFTDYSINWVKRAPGKGLKWMGWINTETREPAYAYDFRG
RFAFSLETSASTAYLQINNLKYEDTATYFCALDYSYAMDYWGQGTSVTVSS;
c)
(SEQ ID NO: 66)
DIVLTQSPASLAVSLGQRATISCRASESVDNYGFSFMHWYQQKPGQPPKLLIYRASNLESGIPAR
FSGSGSRTDFALTINPVETDDVATYYCQQSNKDPRTFGGGTKLEIKGSTSGSGKPGSGEGSTKGQI
QLVQSGPELKKPGETVKISCKASGYTFTDYSINWVKRAPGKGLKWMGWINTETREPAYAYDFR
GRFAFSLETSASTAYLQINNLKYEDTATYFCALDYSYAMDYWGQGTSVTVSS;
and
d)
(SEQ ID NO: 67)
DIVLTQSPASLAMSLGKRATISCRASESVSVIGAHLIHWYQQKPGQPPKLLIYLASNLETGVPAR
FSGSGSGTDFTLTIDPVEEDDVAIYSCLQSRIFPRTFGGGTKLEIKGSTSGSGKPGSGEGSTKGQIQL
VQSGPELKKPGETVKISCKASGYTFTDYSINWVKRAPGKGLKWMGWINTETREPAYAYDFRGR
FAFSLETSASTAYLQINNLKYEDTATYFCALDYSYAMDYWGQGTSVTVSS.

Embodiment 242. The method of any one of embodiments 222-225, wherein the CAR comprises one or more single-domain binding regions specific for B-cell maturation antigen (BCMA), wherein each single-domain binding region comprises an amino acid sequence selected from the group consisting of:

a)
(SEQ ID NO: 68)
QVKLEESGGGLVQAGRSLRLSCAASEHTFSSHVMGWFRQAPGKERESV
AVIGWRDISTSYADSVKGRFTISRDNAKKTLYLQMNSLKPEDTAVYYC
AARRIDAADFDSWGQGTQVTVSS;
and
b)
(SEQ ID NO: 69)
EVQLVESGGGLVQAGGSLRLSCAASGRTFTMGWFRQAPGKEREFVAAI
SLSPTLAYYAESVKGRFTISRDNAKNTVVLQMNSLKPEDTALYYCAAD
RKSVMSIRPDYWGQGTQVTVSS.

Embodiment 243. The method of any one of embodiments 222-225, wherein the CAR comprises an antigen-binding domain specific for B-cell maturation antigen (BCMA), wherein the binding domain comprises the amino acid sequence of

(SEQ ID NO: 70)
MGSWSEFWVRLGAIRERLDALGGSEAELAAFEKEIAAFESELQAYKGKG
NPEVEKLRYTAATIRRFLQAYRHN.

Embodiment 244. The method of any one of embodiments 237-243, wherein the CAR comprises an amino acid sequence selected from the group consisting of:

a)
(SEQ ID NO: 71)
MALPVTALLLPLALLLHAARPQVQLVESGGGLVQPGGSLRLSCAASGFTFTNHAMSWVRQAP
GKGLELVSSISGNGRTTYYADSVKGRFTISRDISKNTLDLQMNSLRAEDTAVYYCAKDGGETLV
DSRGQGTLVTVSSFVPVFLPAKPTTTPAPRPPTPAPTIASQPLSLRPEACRPAAGGAVHTRGLDFA
CDIYIWAPLAGTCGVLLLSLVITLYCNHRNRSKRSRLLHSDYMNMTPRRPGPTRKHYQPYAPPR
DFAAYRSRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR;
b)
(SEQ ID NO: 72)
MALPVTALLLPLALLLHAARPQVQLVESGGGLVQPGGSLRLSCAASGFTFSSHAMTWVRQAP
GKGLEWVAAISGSGDFTHYADSVKGRFTISRDNSKNTVSLQMNNLRAEDTAVYYCAKDEDGGS
LLGYRGQGTLVTVSSFVPVFLPAKPTTTPAPRPPTPAPTIASQPLSLRPEACRPAAGGAVHTRGLD
FACDIYIWAPLAGTCGVLLLSLVITLYCNHRNRSKRSRLLHSDYMNMTPRRPGPTRKHYQPYAPP
RDFAAYRSRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR;
c)
(SEQ ID NO: 73)
MALPVTALLLPLALLLHAARPEVQLLESGGGLVQPGGSLRLSCAASGFTFSSYAMSWVRQAPG
KGLEWVSSISGSGDYIYYADSVKGRFTISRDISKNTLYLQMNSLRAEDTAVYYCAKEGTGANSSL
ADYRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNRSKRSRLLHSDYMNMTPRRPGPTRKHYQPYAPP
RDFAAYRSRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR;
d)
(SEQ ID NO: 74)
MALPVTALLLPLALLLHAARPEVQLLESGGGLIQPGGSLRLSCAASGFTFSSHAMTWVRQAPG
KGLEWVSAISGSGDYTHYADSVKGRFTISRDNSKNTVYLQMNSLRAEDSAVYYCAKDEDGGSL
LGHRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNRSKRSRLLHSDYMNMTPRRPGPTRKHYQPYAPP
RDFAAYRSRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR;
e)
(SEQ ID NO: 75)
MALPVTALLLPLALLLHAARPQVQLVESGGGLVQPGGSLRLSCAASGFTFTNHAMSWVRQAP
GKGLELVSSISGNGRTTYYADSVKGRFTISRDISKNTLDLQMNSLRAEDTAVYYCAKDGGETLV
DSRGQGTLVTVSSFVPVFLPAKPTTTPAPRPPTPAPTIASQPLSLRPEACRPAAGGAVHTRGLDFA
CDIYIWAPLAGTCGVLLLSLVITLYCNHRNKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR;
f)
(SEQ ID NO: 76)
MALPVTALLLPLALLLHAARPQVQLVESGGGLVQPGGSLRLSCAASGFTFSSHAMTWVRQAP
GKGLEWVAAISGSGDFTHYADSVKGRFTISRDNSKNTVSLQMNNLRAEDTAVYYCAKDEDGGS
LLGYRGQGTLVTVSSFVPVFLPAKPTTTPAPRPPTPAPTIASQPLSLRPEACRPAAGGAVHTRGLD
FACDIYIWAPLAGTCGVLLLSLVITLYCNHRNKRGRKKLLYIFKQPFMRPVQTTQEEDGCSCRFP
EEEEGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR;
g)
(SEQ ID NO: 77)
MALPVTALLLPLALLLHAARPEVQLLESGGGLVQPGGSLRLSCAASGFTFSSYAMSWVRQAPG
KGLEWVSSISGSGDYIYYADSVKGRFTISRDISKNTLYLQMNSLRAEDTAVYYCAKEGTGANSSL
ADYRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNKRGRKKLLYIFKQPFMRPVQTTQEEDGCSCRFPE
EEEGGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR;
h)
(SEQ ID NO: 78)
MALPVTALLLPLALLLHAARPEVQLLESGGGLIQPGGSLRLSCAASGFTFSSHAMTWVRQAPG
KGLEWVSAISGSGDYTHYADSVKGRFTISRDNSKNTVYLQMNSLRAEDSAVYYCAKDEDGGSL
LGHRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNKRGRKKLLYIFKQPFMRPVQTTQEEDGCSCRFPE
EEEGGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR;
i)
(SEQ ID NO: 79)
MALPVTALLLPLALLLHAARPQVQLVESGGGLVQPGGSLRLSCAASGFTFTNHAMSWVRQAP
GKGLELVSSISGNGRTTYYADSVKGRFTISRDISKNTLDLQMNSLRAEDTAVYYCAKDGGETLV
DSRGQGTLVTVSSFVPVFLPAKPTTTPAPRPPTPAPTIASQPLSLRPEACRPAAGGAVHTRGLDFA
CDIYIWAPLAGTCGVLLLSLVITLYCNHRNCWLTKKKYSSSVHDPNGEYMFMRAVNTAKKSRL
TDVTLRVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR;
j)
(SEQ ID NO: 80)
MALPVTALLLPLALLLHAARPQVQLVESGGGLVQPGGSLRLSCAASGFTFSSHAMTWVRQAPG
KGLEWVAAISGSGDFTHYADSVKGRFTISRDNSKNTVSLQMNNLRAEDTAVYYCAKDEDGGSL
LGYRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNCWLTKKKYSSSVHDPNGEYMFMRAVNTAKKSR
LTDVTLRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR;
k)
(SEQ ID NO: 81)
MALPVTALLLPLALLLHAARPEVQLLESGGGLVQPGGSLRLSCAASGFTFSSYAMSWVRQAPG
KGLEWVSSISGSGDYIYYADSVKGRFTISRDISKNTLYLQMNSLRAEDTAVYYCAKEGTGANSSL
ADYRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNCWLTKKKYSSSVHDPNGEYMFMRAVNTAKKSR
LTDVTLRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR;
l)
(SEQ ID NO: 82)
MALPVTALLLPLALLLHAARPEVQLLESGGGLIQPGGSLRLSCAASGFTFSSHAMTWVRQAPG
KGLEWVSAISGSGDYTHYADSVKGRFTISRDNSKNTVYLQMNSLRAEDSAVYYCAKDEDGGSL
LGHRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNCWLTKKKYSSSVHDPNGEYMFMRAVNTAKKSR
LTDVTLRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR;
m)
(SEQ ID NO: 83)
MALPVTALLLPLALLLHAARPDIVLTQSPPSLAMSLGKRATISCRASESVTILGSHLIYWYQQK
PGQPPTLLIQLASNVQTGVPARFSGSGSRTDFTLTIDPVEEDDVAVYYCLQSRTIPRTFGGGTKLEI
KGSTSGSGKPGSGEGSTKGQIQLVQSGPELKKPGETVKISCKASGYTFRHYSMNWVKQAPGKGL
KWMGRINTESGVPIYADDFKGRFAFSVETSASTAYLVINNLKDEDTASYFCSNDYLYSLDFWGQ
GTALTVSSFVPVFLPAKPTTTPAPRPPTPAPTIASQPLSLRPEACRPAAGGAVHTRGLDFACDIYIW
APLAGTCGVLLLSLVITLYCNHRNRSKRSRLLHSDYMNMTPRRPGPTRKHYQPYAPPRDFAAYR
SRVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR;
n)
(SEQ ID NO: 84)
MALPVTALLLPLALLLHAARPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSFVPVFLPAKPTTTPAPRPPTPAPTIASQPLSLRPEACRPAAGGAVHTRGLDFACDIYIW
APLAGTCGVLLLSLVITLYCNHRNRSKRSRLLHSDYMNMTPRRPGPTRKHYQPYAPPRDFAAYR
SRVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR;
o)
(SEQ ID NO: 85)
MALPVTALLLPLALLLHAARPDIVLTQSPPSLAMSLGKRATISCRASESVTILGSHLIYWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTHYSMNWVKQAPGKGLK
WMGRINTETGEPLYADDFKGRFAFSLETSASTAYLVINNLKNEDTATFFCSNDYLYSCDYWGQG
TTLTVSSFVPVFLPAKPTTTPAPRPPTPAPTIASQPLSLRPEACRPAAGGAVHTRGLDFACDIYIWA
PLAGTCGVLLLSLVITLYCNHRNRSKRSRLLHSDYMNMTPRRPGPTRKHYQPYAPPRDFAAYRS
RVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR;
p)
(SEQ ID NO: 86)
MLLLVTSLLLCELPHPAFLLIPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSAAAFVPVFLPAKPTTTPAPRPPTPAPTIASQPLSLRPEACRPAAGGAVHTRGLDFACDI
YIWAPLAGTCGVLLLSLVITLYCNHRNRSKRSRLLHSDYMNMTPRRPGPTRKHYQPYAPPRDFA
AYRSRVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR;
q)
(SEQ ID NO: 87)
MALPVTALLLPLALLLHAARPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSAAAFVPVFLPAKPTTTPAPRPPTPAPTIASQPLSLRPEACRPAAGGAVHTRGLDFACDI
YIWAPLAGTCGVLLLSLVITLYCNHRNRSKRSRLLHSDYMNMTPRRPGPTRKHYQPYAPPRDFA
AYRSRVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR;
r)
(SEQ ID NO: 88)
MALPVTALLLPLALLLHAARPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSAAAFVPVFLPAKPTTTPAPRPPTPAPTIASQPLSLRPEACRPAAGGAVHTRGLDFACDI
YIWAPLAGTCGVLLLSLVITLYCNHRNRFSVVKRGRKKLLYIFKQPFMRPVQTTQEEDGCSCRFP
EEEEGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR;
s)
(SEQ ID NO: 89)
MALPVTALLLPLALLLHAARPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSAAAFVPVFLPAKPTTTPAPRPPTPAPTIASQPLSLRPEACRPAAGGAVHTRGLDFACDI
YIWAPLAGTCGVLLLSLVITLYCNHRNRRDQRLPPDAHKPPGGGSFRTPIQEEQADAHSTLAKIR
VKFSRSADAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR;
t)
(SEQ ID NO: 90)
MALPVTALLLPLALLLHAARPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSAAAFVPVFLPAKPTTTPAPRPPTPAPTIASQPLSLRPEACRPAAGGAVHTRGLDFACDI
YIWAPLAGTCGVLLLSLVITLYCNHRNRFSVVKRGRKKLLYIFKQPFMRPVQTTQEEDGCSCRFP
EEEEGGCELRRDQRLPPDAHKPPGGGSFRTPIQEEQADAHSTLAKIRVKFSRSADAPAYQQGQNQ
LYNELNLGRREEYDVLDKRRGRDPEMGGKPRRKNPQEGLYNELQKDKMAEAYSEIGMKGERR
RGKGHDGLYQGLSTATKDTYDALHMQALPPR;
u)
(SEQ ID NO: 91)
MALPVTALLLPLALLLHAARPQVKLEESGGGLVQAGRSLRLSCAASEHTFSSHVMGWFRQAP
GKERESVAVIGWRDISTSYADSVKGRFTISRDNAKKTLYLQMNSLKPEDTAVYYCAARRIDAAD
FDSWGQGTQVTVSSGGGGSEVQLVESGGGLVQAGGSLRLSCAASGRTFTMGWFRQAPGKEREF
VAAISLSPTLAYYAESVKGRFTISRDNAKNTVVLQMNSLKPEDTALYYCAADRKSVMSIRPDYW
GQGTQVTVSSTSTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR;
and
v)
(SEQ ID NO: 92)
MAFLWLLSCWALLGTTFGDYKDDDDKGGGGSGGGGSMGSWSEFWVRLGAIRERLDALGGS
EAELAAFEKEIAAFESELQAYKGKGNPEVEKLRYTAATIRRFLQAYRHNGGGGSGGGGSGTTTP
APRPPTPAPTIASQPLSLRPEACRPAAGGAVHTRGLDFACDIYIWAPLAGTCGVLLLSLVITLYCK
RGRKKLLYIFKQPFMRPVQTTQEEDGCSCRFPEEEEGGCELRVKFSRSADAPAYKQGQNQLYNE
LNLGRREEYDVLDKRRGRDPEMGGKPRRKNPQEGLYNELQKDKMAEAYSEIGMKGERRRGKG
HDGLYQGLSTATKDTYDALHMQALPPR.

Embodiment 245. The method of any one of embodiments 227-244, wherein the human subject is administered a dose of CAR+ cells that ranges from approximately 1.0×105 to approximately 5.0×106 CAR+ cells per kilogram of body weight of the human subject, or from approximately 4.0×105 to 1.0×109 CAR+ cells.

Embodiment 246. The method of any one of embodiments 1-245, wherein the one or more cells comprising a chimeric receptor are selected from the group consisting of: T cells, Natural Killer (NK) cells, cytotoxic T lymphocytes (CTLs), regulatory T cells, and Natural Killer T (NKT) cells.

Embodiment 247. The method of embodiment 246, wherein the one or more cells comprising a chimeric receptor are from an allogeneic donor and have been genetically modified to reduce risk of graft versus host disease (GVHD) and/or to prevent allogeneic rejection.

Embodiment 248. The method of any one of embodiments 1-247, wherein the hematologic malignancy is selected from the group consisting of: acute lymphoblastic leukemia (ALL), acute myelogenous leukemia (AML), chronic myelogenous leukemia (CML), multiple myeloma, lymphoma, Hodgkin's lymphoma, non-Hodgkin lymphoma, myelodysplastic syndrome, myelofibrosis, and blastic plasmacytoid dendritic cell neoplasm (BPDCN).

Embodiment 249. The method of embodiment 248, wherein the ALL is B-cell ALL.

Embodiment 250. The method of embodiment 249, wherein the human subject is in complete response with a history of chemotherapy refractory disease.

Embodiment 251. The method of embodiment 250, wherein the chemotherapy refractory disease comprises progression or stable disease after one line of chemotherapy.

Embodiment 252. The method of embodiment 249, wherein the human subject is in complete response with CRLF2 rearrangement, Ph-like phenotype, MLL/KMT2a rearrangement, and/or hypodiploid karyotype.

Embodiment 253. The method of embodiment 252, wherein the Ph-like phenotype comprises Ph+ ALL.

Embodiment 254. The method of embodiment 249, wherein the human subject has persistent or relapsed minimal residual disease (MRD).

Embodiment 255. The method of embodiment 249, wherein the human subject has active B-cell ALL.

Embodiment 256. The method of embodiment 255, wherein the human subject has approximately 5% or greater bone marrow blasts, circulating blasts, and/or extramedullary disease.

Embodiment 257. The method of any one of embodiments 249-256, wherein the B-cell ALL is CD19+.

Embodiment 258. The method of any one of embodiments 1-257, wherein incidence of immune effector cell-associated neurotoxicity syndrome (ICANS) decreases after administration of the multi-component cellular therapy, relative to a corresponding human subject that has been administered only a CAR-T monotherapy.

Embodiment 259. The method of any one of embodiments 1-258, wherein the multi-component cellular therapy prevents development of immune effector cell-associated neurotoxicity syndrome (ICANS) for a period that ranges from approximately 1 month to approximately 6 months, or longer.

Embodiment 260. The method of any one of embodiments 1-259, wherein incidence of cytokine release syndrome (CRS) decreases after administration of the multi-component cellular therapy, relative to a corresponding human subject that has been administered only a CAR-T monotherapy.

Embodiment 261. The method of any one of embodiments 1-260, wherein severity of cytokine release syndrome (CRS) decreases after administration of the multi-component cellular therapy, relative to a corresponding human subject that has been administered only a CAR-T monotherapy.

Embodiment 262. The method of any one of embodiments 1-261, wherein incidence of graft vs host disease (GVHD) decreases after administration of the multi-component cellular therapy, relative to a corresponding human subject that has been administered only an allogeneic CAR-T monotherapy, or relative to a corresponding human subject that has been administered a standard myeloablative allogeneic hematopoietic stem cell transplant (alloHSCT) without a CAR-T therapy.

Embodiment 263. The method of any one of embodiments 1-262, wherein the multi-component cellular therapy prevents development of graft vs host disease (GVHD) for a period that ranges from approximately 1 month to approximately 6 months, or longer.

Embodiment 264. The method of any one of embodiments 1-263, wherein incidence of complete response increases after administration of the multi-component cellular therapy, relative to a corresponding human subject that has been administered only a CAR-T monotherapy.

Embodiment 265. The method of any one of embodiment 1-264, wherein incidence of transplant related mortality (TRM) decreases after administration of the multi-component cellular therapy, relative to a corresponding human subject that has been administered only a CAR-T monotherapy.

Embodiment 266. The method of any one of embodiment 1-265, wherein incidence of minimal residual disease (MRD) decreases after administration of the multi-component cellular therapy, relative to a corresponding human subject that has been administered only a CAR-T monotherapy.

Embodiment 267. The method of any one of embodiments 1-266, wherein the multi-component cellular therapy prevents development of minimal residual disease (MRD) for a period that ranges from approximately 1 month to approximately 6 months, or longer.

Embodiment 268. The method of any one of embodiments 1-267, wherein incidence of Grade III-IV acute GVHD (aGVHD) 42 or more days after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 269. The method of any one of embodiments 1-268, wherein incidence of relapse decreases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 270. The method of any one of embodiments 1-269, wherein incidence of disease progression decreases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 271. The method of any one of embodiments 1-270, wherein incidence of non-relapse mortality (NRM) decreases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 272. The method of any one of embodiments 1-271, wherein incidence of secondary graft failure decreases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 273. The method of any one of embodiments 1-272, wherein progression-free survival (PRS) increases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 274. The method of any one of embodiments 1-273, wherein overall survival increases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 275. The method of any one of embodiments 1-274, wherein incidence of Grade 3 or higher infectious disease complications decreases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 276. The method of any one of embodiments 1-275, wherein the human subject is approximately 3 months of age or older, approximately 6 months of age or older, approximately 9 months of age or older, approximately 12 months of age or older, approximately 18 months of age or older, approximately 2 years of age or older, approximately 3 years of age or older, approximately 4 years of age or older, approximately 5 years of age or older, approximately 6 years of age or older, approximately 7 years of age or older, approximately 8 years of age or older, approximately 9 years of age or older, approximately 10 years of age or older, approximately 11 years of age or older, approximately 12 years of age or older, approximately 13 years of age or older, approximately 14 years of age or older, approximately 15 years of age or older, approximately 16 years of age or older, approximately 17 years of age or older, approximately 18 years of age or older, approximately 19 years of age or older, approximately 20 years of age or older, or approximately 21 years of age or older.

Embodiment 277. The method of any one of embodiments 1-276, wherein the human subject is between approximately 18 years of age to approximately 65 years of age.

Embodiment 278. The method of any one of embodiments 1-276, wherein the human subject is between approximately 18 years of age to approximately 75 years of age.

Embodiment 279. A method of treating a human subject having or suspected of having a hematologic condition, the method comprising:
(a) administering a single conditioning regimen; and
(b) administering two or more cell therapies.

Embodiment 280. The method of embodiment 279, wherein the single condition regimen comprises any one of the conditioning regimens of any one of embodiments 182-220.

Embodiment 281. The method of embodiment 279 or embodiment 280, wherein the two or more cell therapies are selected from the group consisting of: a hematopoietic stem cell transplant (HSCT), a chimeric antigen receptor (CAR) cell therapy, an engineered T cell receptor (TCR) therapy, a tumor-infiltrating lymphocyte (TIL) therapy, a natural killer (NK) cell therapy, a regenerative cell therapy, and any combination thereof.

Embodiment 282. The method of embodiment 281, wherein the HSCT therapy is the HSCT therapy of any one of embodiments 1-278.

Embodiment 283. The method of embodiment 281 or embodiment 282, wherein the CAR cell therapy is the CAR cell therapy of any one of embodiments 1-246.

Embodiment 284. A multi-component cellular therapy product comprising:
(a) one or more allogeneic cells comprising a chimeric receptor; and
(b) an immune-modulating cell therapy product,
wherein the one or more allogeneic cells and/or one or more cells of the immune-modulating cell therapy product are from an HLA-compatible donor, relative to a human subject receiving the multi-component cellular therapy product.

Embodiment 285. The multi-component cellular therapy product of embodiment 284, wherein the HLA-compatible donor is an allogeneic HLA-matched donor, relative to the human subject.

Embodiment 286. The cellular therapy product of embodiment 284, wherein the HLA-compatible donor is an allogeneic HLA-mismatched donor, relative to the human subject.

Embodiment 287. The multi-component cellular therapy product of any one of embodiments 284-286, wherein the one or more allogeneic cells have not been genetically modified to reduce risk of graft versus host disease (GVHD) and/or to prevent allogeneic rejection with respect to the human subject.

Embodiment 288. The multi-component cellular therapy product of any one of embodiments 284-287, wherein the immune-modulating cell therapy product comprises a regulatory T cell therapy.

Embodiment 289. The multi-component cellular therapy product of any one of embodiments 284-287, wherein the immune-modulating cell therapy product comprises an allogeneic hematopoietic stem cell transplant (HSCT).

Embodiment 290. The multi-component cellular therapy product of embodiment 289, wherein the allogeneic HSCT comprises a population of CD34+ hematopoietic stem and progenitor cells (HSPCs).

Embodiment 291. The multi-component cellular therapy product of embodiment 289 or embodiment 290, wherein the allogeneic HSCT comprises a population of CD4+CD25+CD127dim regulatory T cells (Tregs).

Embodiment 292. The multi-component cellular therapy product of any one of embodiments 289-291, wherein the allogeneic HSCT comprises a population of HSPCs and a population of Tregs.

Embodiment 293. The multi-component cellular therapy product of any one of embodiments 289-292, wherein the allogeneic HSCT comprises a population of CD45RA− memory T cells (Tmems).

Embodiment 294. The multi-component cellular therapy product of any one of embodiments 289-293, wherein the allogeneic HSCT comprises a population of HSPCs and a population of Tmems.

Embodiment 295. The multi-component cellular therapy product of any one of embodiments 289-293, wherein the allogeneic HSCT comprises a population of Treg and a population of Tmems.

Embodiment 296. The multi-component cellular therapy product of any one of embodiments 289-295, wherein the allogeneic HSCT comprises a population of HSPCs, a population of Treg, and a population of Tmems.

Embodiment 297. The multi-component cellular therapy product of any one of embodiments 289-296, wherein the allogeneic HSCT has been depleted of naïve conventional CD3+CD25−CD45RA+ T cells.

Embodiment 298. The multi-component cellular therapy product of any one of embodiments 289-297, wherein the allogeneic HSCT comprises a population of conventional CD3⁺ T cells (Tcons), or any combination thereof.

Embodiment 299. The multi-component cellular therapy product of any one of embodiments 289-298, wherein allogeneic HSCT comprises a population of HSPCs and a population of Tcons.

Embodiment 300. The multi-component cellular therapy product of any one of embodiments 289-298, wherein allogeneic HSCT comprises a population of Tregs and a population of Tcons.

Embodiment 301. The multi-component cellular therapy product of any one of embodiments 289-298, wherein allogeneic HSCT comprises a population of Tmems and a population of Tcons.

Embodiment 302. The multi-component cellular therapy product of any one of embodiments 289-300, wherein allogeneic HSCT comprises a population of HSPCs, a population of Tregs, and a population of Tcons.

Embodiment 303. The multi-component cellular therapy product of any one of embodiments 289-302, wherein allogeneic HSCT comprises a population of HSPCs, a population of Tregs, a population of Tmems, and a population of Tcons.

Embodiment 304. The multi-component cellular therapy product of embodiment 289, wherein the allogeneic HSCT is a T cell-depleted allogeneic HSCT.

Embodiment 305. The multi-component cellular therapy product of any one of embodiments 284-304, wherein the one or more cells comprising the chimeric receptor comprise a pharmaceutical composition comprising an isolated nucleic acid encoding the chimeric receptor.

Embodiment 306. A multi-component cellular therapy product comprising:
(a) a population of CD34+ hematopoietic stem and progenitor cells (HSPCs); and
(b) one or more cells comprising a chimeric receptor or a pharmaceutical composition comprising an isolated nucleic acid encoding the chimeric receptor.

Embodiment 307. The multi-component cellular therapy product of embodiment 306, further comprising a population of CD4+CD25+CD127dim regulatory T cells (Tregs).

Embodiment 308. The multi-component cellular therapy product of embodiment 307, wherein the population of Tregs comprises the one or more cells comprising a chimeric receptor.

Embodiment 309. The multi-component cellular therapy product of any one of embodiments 305-308, further comprising a population of CD45RA− memory T cells (Tmems).

Embodiment 310. The multi-component cellular therapy product of embodiment 309, wherein the population of Tmems comprises the one or more cells comprising a chimeric receptor.

Embodiment 311. The multi-component cellular therapy product of any one of embodiments 306-310, further comprising a population of conventional CD3+ T cells (Tcons).

Embodiment 312. The multi-component cellular therapy product of embodiment 311, wherein the population of Tcons comprises the one or more cells comprising a chimeric receptor.

Embodiment 313. A multi-component cellular therapy product comprising a population of CD34+ hematopoietic stem and progenitor cells (HSPCs), wherein the population HSPCs comprises one or more cells comprising a chimeric receptor.

Embodiment 314. The multi-component cellular therapy product of embodiment 313, further comprising a population of CD4+CD25+CD127dim regulatory T cells (Tregs).

Embodiment 315. The multi-component cellular therapy product of embodiment 313 or embodiment 314, further comprising a population of CD45RA− memory T cells (Tmems).

Embodiment 316. The multi-component cellular therapy product of any one of embodiments 313-315, wherein the product further comprises a population of conventional CD3+ T cells (Tcons).

Embodiment 317. A multi-component cellular therapy product comprising:
(a) a population of CD4+CD25+CD127dim regulatory T cells (Tregs); and
(b) one or more cells comprising a chimeric receptor or a pharmaceutical composition comprising an isolated nucleic acid encoding the chimeric receptor.

Embodiment 318. The multi-component cellular therapy product of embodiment 317, further comprising a population of CD34+ hematopoietic stem and progenitor cells (HSPCs).

Embodiment 319. The multi-component cellular therapy product of embodiment 318, wherein the population of HSPCs comprises the one or more cells comprising a chimeric receptor.

Embodiment 320. The multi-component cellular therapy product of any one of embodiments 317-319, further comprising a population of CD45RA− memory T cells (Tmems).

Embodiment 321. The multi-component cellular therapy product of embodiment 320, wherein the population of Tmems comprises the one or more cells comprising a chimeric receptor.

Embodiment 322. The multi-component cellular therapy product of any one of embodiments 317-321, further comprising a population of conventional CD3+ T cells (Tcons).

Embodiment 323. The multi-component cellular therapy product of embodiment 322, wherein the population of Tcons comprises the one or more cells comprising a chimeric receptor.

Embodiment 324. A multi-component cellular therapy product comprising a population of CD4+CD25+CD127dim regulatory T cells (Tregs),
wherein the population of Tregs comprises one or more cells comprising a chimeric receptor.

Embodiment 325. The multi-component cellular therapy product of embodiment 324, further comprising a population of CD34+ hematopoietic stem and progenitor cells (HSPCs).

Embodiment 326. The multi-component cellular therapy product of embodiment 324 or embodiment 325, further comprising a population of CD45RA− memory T cells (Tmems).

Embodiment 327. The multi-component cellular therapy product of any one of embodiments 324-326, further comprising a population of conventional CD3+ T cells (Tcons).

Embodiment 328. A multi-component cellular therapy product comprising:
(a) a population of CD45RA− memory T cells (Tmems); and
(b) one or more cells comprising a chimeric receptor or a pharmaceutical composition comprising an isolated nucleic acid encoding the chimeric receptor.

Embodiment 329. The multi-component cellular therapy product of embodiment 328, wherein the multi-component cellular therapy further comprises a population of CD34+ hematopoietic stem and progenitor cells (HSPCs).

Embodiment 330. The multi-component cellular therapy product of embodiment 329, wherein the population of HSPCs comprises the one or more cells comprising a chimeric receptor.

Embodiment 331. The multi-component cellular therapy product of any one of embodiments 328-330, wherein the multi-component cellular therapy further comprises a population of CD4+CD25+CD127dim regulatory T cells (Tregs).

Embodiment 332. The multi-component cellular therapy product of embodiment 331, wherein the population of Tregs comprises the one or more cells comprising a chimeric receptor.

Embodiment 333. The multi-component cellular therapy product of any one of embodiments 328-332, wherein the multi-component cellular therapy further comprises a population of conventional CD3+ T cells (Tcons).

Embodiment 334. The multi-component cellular therapy product of embodiment 333, wherein the population of Tcons comprises the one or more cells comprising a chimeric receptor.

Embodiment 335. A multi-component cellular therapy product comprising a population of CD45RA− memory T cells (Tmems),
wherein the population of Tmems comprises one or more cells comprising a chimeric receptor.

Embodiment 336. The multi-component cellular therapy product of embodiment 335, further comprising a population of CD34+ hematopoietic stem and progenitor cells (HSPCs).

Embodiment 337. The multi-component cellular therapy product of embodiment 335 or embodiment 336, further comprising a population of CD4+CD25+CD127dim regulatory T cells (Tregs).

Embodiment 338. The multi-component cellular therapy product of any one of embodiments 335-337, further comprising a population of conventional CD3⁺ T cells (Tcons).

Embodiment 339. A multi-component cellular therapy product comprising:
(a) a population of CD34+ hematopoietic stem and progenitor cells (HSPCs);
(b) a population of CD4+CD25+CD127dim regulatory T cells (Tregs); and
(c) one or more cells comprising a chimeric receptor or a pharmaceutical composition comprising an isolated nucleic acid encoding the chimeric receptor.

Embodiment 340. The multi-component cellular therapy product of embodiment 339, wherein the population of HSPCs comprises the one or more cells comprising a chimeric receptor.

Embodiment 341. The multi-component cellular therapy product of embodiment 339, wherein the population of Tregs comprises the one or more cells comprising a chimeric receptor.

Embodiment 342. The multi-component cellular therapy product of any one of embodiments 339-341, further comprising a population of CD45RA− memory T cells (Tmems).

Embodiment 343. The multi-component cellular therapy product of embodiment 342, wherein the population of Tmems comprises the one or more cells comprising a chimeric receptor.

Embodiment 344. The multi-component cellular therapy product of any one of embodiments 339-343, further comprising a population of conventional CD3+ T cells (Tcons).

Embodiment 345. The multi-component cellular therapy product of embodiment 344, wherein the population of Tcons comprises the one or more cells comprising a chimeric receptor.

Embodiment 346. A multi-component cellular therapy product comprising:
(a) a population of CD34+ hematopoietic stem and progenitor cells (HSPCs); and
(b) a population of CD4+CD25+CD127dim regulatory T cells (Tregs),
wherein the population of HSPCs, the population of Tregs, or the population of HSPCs and the population of Tregs comprises one or more cells comprising a chimeric receptor.

Embodiment 347. The multi-component cellular therapy product of embodiment 346, further comprising a population of CD45RA− memory T cells (Tmems).

Embodiment 348. The multi-component cellular therapy product of embodiment 346 or embodiment 347, further comprising a population of conventional CD3+ T cells (Tcons).

Embodiment 349. A multi-component cellular therapy product comprising:
(a) a population of CD34+ hematopoietic stem and progenitor cells (HSPCs);
(b) a population of CD45RA− memory T cells (Tmems); and
(c) one or more cells comprising a chimeric receptor or a pharmaceutical composition comprising an isolated nucleic acid encoding the chimeric receptor.

Embodiment 350. The multi-component cellular therapy product of embodiment 349, wherein the population of HSPCs comprises the one or more cells comprising a chimeric receptor.

Embodiment 351. The multi-component cellular therapy product of embodiment 349, wherein the population of Tmems comprises the one or more cells comprising a chimeric receptor.

Embodiment 352. The multi-component cellular therapy product of any one of embodiments 349-351, further comprising a population of CD4+CD25+CD127dim regulatory T cells (Tregs).

Embodiment 353. The multi-component cellular therapy product of embodiment 352, wherein the population of Tregs comprises the one or more cells comprising a chimeric receptor.

Embodiment 354. The multi-component cellular therapy product of any one of embodiments 349-353, further comprising a population of conventional CD3+ T cells (Tcons).

Embodiment 355. The multi-component cellular therapy product of embodiment 354, wherein the population of Tcons comprises the one or more cells comprising a chimeric receptor.

Embodiment 356. A multi-component cellular therapy product comprising:
(a) a population of CD34+ hematopoietic stem and progenitor cells (HSPCs); and
(b) a population of CD45RA− memory T cells (Tmems); and
wherein the population of HSPCs, the population of Tmems, or the population of HSPCs and the population of Tmems comprises one or more cells comprising a chimeric receptor.

Embodiment 357. The multi-component cellular therapy product of embodiment 356, further comprising a population of CD4+CD25+CD127dim regulatory T cells (Tregs).

Embodiment 358. The multi-component cellular therapy product of embodiment 356 or embodiment 357, further comprising a population of conventional CD3⁺ T cells (Tcons).

Embodiment 359. A multi-component cellular therapy product comprising:
(a) a population of CD34+ hematopoietic stem and progenitor cells (HSPCs);
(b) a population of conventional CD3+ T cells (Tcons); and
(c) one or more cells comprising a chimeric receptor or a pharmaceutical composition comprising an isolated nucleic acid encoding the chimeric receptor.

Embodiment 360. The multi-component cellular therapy product of embodiment 359, wherein the population of HSPCs comprises the one or more cells comprising a chimeric receptor.

Embodiment 361. The multi-component cellular therapy product of embodiment 359, wherein the population of Tcons comprises the one or more cells comprising a chimeric receptor.

Embodiment 362. The multi-component cellular therapy product of any one of embodiments 359-361, further comprising a population of CD4+CD25+CD127dim regulatory T cells (Tregs).

Embodiment 363. The multi-component cellular therapy product of embodiment 362, wherein the population of Tregs comprises the one or more cells comprising a chimeric receptor.

Embodiment 364. The multi-component cellular therapy product of any one of embodiments 359-363, further comprising a population of CD45RA− memory T cells (Tmems).

Embodiment 365. The multi-component cellular therapy product of embodiment 364, wherein the population of Tmems comprises the one or more cells comprising a chimeric receptor.

Embodiment 366. A multi-component cellular therapy product comprising:
(a) a population of CD34+ hematopoietic stem and progenitor cells (HSPCs); and
(b) a population of conventional CD3+ T cells (Tcons),
wherein the population of HSPCs, the population of Tcons, or the population of HSPCs and the population of Tcons comprises one or more cells comprising a chimeric receptor.

Embodiment 367. The multi-component cellular therapy product of embodiment 366, further comprising a population of CD4+CD25+CD127dim regulatory T cells (Tregs).

Embodiment 368. The multi-component cellular therapy product of embodiment 366 or embodiment 367, further comprising a population of CD45RA− memory T cells (Tmems).

Embodiment 369. A multi-component cellular therapy product comprising:
(a) a population of CD34+ hematopoietic stem and progenitor cells (HSPCs);
(b) a population of CD4+CD25+CD127dim regulatory T cells (Tregs);
(c) a population of conventional CD3+ T cells (Tcons); and
(d) one or more cells comprising a chimeric receptor or a pharmaceutical composition comprising an isolated nucleic acid encoding the chimeric receptor.

Embodiment 370. The multi-component cellular therapy product of embodiment 369, wherein the population of HSPCs comprises the one or more cells comprising a chimeric receptor.

Embodiment 371. The multi-component cellular therapy product of embodiment 369, wherein the population of Tregs comprises the one or more cells comprising a chimeric receptor.

Embodiment 372. The multi-component cellular therapy product of embodiment 369, wherein the population of Tcons comprises the one or more cells comprising a chimeric receptor.

Embodiment 373. The multi-component cellular therapy product of any one of embodiments 369-372, further comprising a population of CD45RA− memory T cells (Tmems).

Embodiment 374. The multi-component cellular therapy product of embodiment 373, wherein the population of Tmems comprises the one or more cells comprising a chimeric receptor.

Embodiment 375. A multi-component cellular therapy product comprising:
(a) a population of CD34+ hematopoietic stem and progenitor cells (HSPCs);
(b) a population of CD4+CD25+CD127dim regulatory T cells (Tregs); and
(c) a population of conventional CD3+ T cells (Tcons),
wherein the population of HSPCs, the population of Tregs, and/or the population of Tcons comprises one or more cells comprising a chimeric receptor.

Embodiment 376. The multi-component cellular therapy product of embodiment 375, further comprising a population of CD45RA− memory T cells (Tmems).

Embodiment 377. A multi-component cellular therapy product comprising:
(a) a population of CD34+ hematopoietic stem and progenitor cells (HSPCs);
(b) a population of CD4+CD25+CD127dim regulatory T cells (Tregs);
(c) a population of CD45RA− memory T cells (Tmems);
(d) a population of conventional CD3+ T cells (Tcons); and
(e) one or more cells comprising a chimeric receptor or a pharmaceutical composition comprising an isolated nucleic acid encoding the chimeric receptor.

Embodiment 378. The multi-component cellular therapy product of embodiment 377, wherein the population of HSPCs comprises the one or more cells comprising a chimeric receptor.

Embodiment 379. The multi-component cellular therapy product of embodiment 377, wherein the population of Tregs comprises the one or more cells comprising a chimeric receptor.

Embodiment 380. The multi-component cellular therapy product of embodiment 377, wherein the population of Tmems comprises the one or more cells comprising a chimeric receptor.

Embodiment 381. The multi-component cellular therapy product of embodiment 377, wherein the population of Tcons comprises the one or more cells comprising a chimeric receptor.

Embodiment 382. A multi-component cellular therapy product comprising:
(a) a population of CD34+ hematopoietic stem and progenitor cells (HSPCs);
(b) a population of CD4+CD25+CD127dim regulatory T cells (Tregs);
(c) a population of CD45RA− memory T cells (Tmems); and
(d) a population of conventional CD3+ T cells (Tcons),
wherein the population of HSPCs, the population of Tregs, the population of Tmems, and/or the population of Tcons comprises one or more cells comprising a chimeric receptor.

Embodiment 383. The multi-component cellular therapy product of any one of embodiments 305, 306, 317, 328, 339, 349, 359, 369, and 377, wherein the pharmaceutical composition comprises a vector comprising the nucleic acid.

Embodiment 384. The multi-component cellular therapy product of embodiment 383, wherein the vector is a viral vector.

Embodiment 385. The multi-component cellular therapy product of embodiment 384, wherein the viral vector is selected from the group consisting of: a lentiviral vector, a retroviral vector, an oncolytic viral vector, an adenoviral vector, an adeno-associated viral (AAV) vector, and a virus-like particle (VLP).

Embodiment 386. The multi-component cellular therapy product of any one of embodiments 305, 306, 317, 328, 339, 349, 359, 369, and 377, wherein the pharmaceutical composition comprises a nanoparticle comprising the nucleic acid.

Embodiment 387. The multi-component cellular therapy product of embodiment 386, wherein the nanoparticle is a lipid nanoparticle (LNP).

Embodiment 388. The multi-component cellular therapy product of any one of embodiments 305, 306, 317, 328, 339, 349, 359, 369, and 377-387, wherein the pharmaceutical composition further comprises a pharmaceutically acceptable carrier, pharmaceutically acceptable excipient, or a combination thereof.

Embodiment 389. A multi-component cellular therapy product comprising:
(a) a population of CD34+ hematopoietic stem and progenitor cells (HSPCs);
(b) a population of CD4+CD25+CD127dim regulatory T cells (Tregs); and
(c) a population of conventional CD3+ T cells (Tcons), wherein the population of Tcons comprises one or more cells comprising a chimeric receptor.

Embodiment 390. The multi-component cellular therapy product of 389, further comprising a population of CD45RA− memory T cells (Tmems).

Embodiment 391. The multi-component cellular therapy product of embodiment 389 or embodiment 390, wherein the population of Tcons has been genetically modified to express the chimeric receptor prior.

Embodiment 392. The multi-component cellular therapy product of embodiment 391, wherein the one or more cells were genetically modified via transfection with a vector comprising a nucleic acid encoding the chimeric receptor.

Embodiment 393. The multi-component cellular therapy product of embodiment 392, wherein the vector is a viral vector selected from the group consisting of a lentiviral vector, a retroviral vector, an oncolytic viral vector, an adenoviral vector, an adeno-associated viral (AAV) vector, and a virus-like particle (VLP).

Embodiment 394. The multi-component cellular therapy product of embodiment 391, wherein the one or more cells were genetically modified via introduction of a nanoparticle comprising a nucleic acid encoding the chimeric receptor.

Embodiment 395. The multi-component cellular therapy product of embodiment 394, wherein the nanoparticle is a lipid nanoparticle (LNP).

Embodiment 396. The multi-component cellular therapy product of embodiment 391, wherein the one or more cells were genetically modified via electroporation of a nucleic acid encoding the chimeric receptor.

Embodiment 397. The multi-component cellular therapy product of any one of embodiments 391-396, wherein the genetically modified Tcons are cryogenically preserved.

Embodiment 398. A multi-component cellular therapy product comprising:
(a) a population of CD34+ hematopoietic stem and progenitor cells (HSPCs), a population of CD4+CD25+CD127dim regulatory T cells (Tregs), a population of conventional CD3+ T cells (Tcons), or any combination thereof; and
(b) a population of tumor-infiltrating lymphocytes (TILs).

Embodiment 399. The multi-component cellular therapy product of embodiment 398, wherein the population of Tcons comprises the population of TILs.

Embodiment 400. The multi-component cellular therapy product of embodiment 398 or embodiment 399, further comprising a population of CD45RA− memory T cells (Tmems).

Embodiment 401. The multi-component cellular therapy product of any one of embodiments 284-400, wherein the Tcons are formulated for administration approximately 12 hours or more after administration of the HSPCs, after administration of the Tregs, after administration of the Tmems, or after administration of any combination of the HSPCs, the Tregs, and the Tmems.

Embodiment 402. The multi-component cellular therapy product of any one of embodiments 284-401, wherein the Tcons are formulated for administration from approximately 24 to approximately 120 hours after administration of the HSPCs, after administration of the Tregs, after administration of the Tmems, or after administration of any combination of the HSPCs, the Tregs, and the Tmems.

Embodiment 403. The multi-component cellular therapy product of any one of embodiments 284-402, wherein the Tcons are formulated for administration from approximately 36 to approximately 72 hours after administration of the HSPCs, after administration of the Tregs, after administration of the Tmems, or after administration of any combination of the HSPCs, the Tregs, and the Tmems.

Embodiment 404. The multi-component cellular therapy product of any one of embodiments 284-403, wherein the Tregs, the Tmems, or the Tregs and the Tmems are formulated for administration from approximately 5 minutes to approximately 5 hours after administration of the HSPCs.

Embodiment 405. The multi-component cellular therapy product of any one of embodiments 284-404, wherein the one or more cells comprising a chimeric receptor are formulated for administration approximately 12 hours or more after administration of the HSPCs, after administration of the Tregs, after administration of the Tmems, or after administration of any combination of the HSPCs, the Tregs, and the Tmems.

Embodiment 406. The multi-component cellular therapy product of any one of embodiments 284-405, wherein the one or more cells comprising a chimeric receptor are formulated for administration from approximately 24 to approximately 120 hours after administration of the HSPCs, after administration of the Tregs, after administration of the Tmems, or after administration of any combination of the HSPCs, the Tregs, and the Tmems.

Embodiment 407. The multi-component cellular therapy product of any one of embodiments 284-406, wherein the one or more cells comprising a chimeric receptor are formulated for administration from approximately 36 to approximately 72 hours after administration of the HSPCs, after administration of the Tregs, after administration of the Tmems, or after administration of any combination of the HSPCs, the Tregs, and the Tmems.

Embodiment 408. The multi-component cellular therapy product of any one of embodiments 284-407, wherein the one or more cells comprising a chimeric receptor are formulated for administration from approximately 5 minutes to approximately 5 hours after administration of the Tcons.

Embodiment 409. The multi-component cellular therapy product of any one of embodiments 284-408, further comprising a pharmaceutical composition comprising one or more doses of a graft vs host disease (GVHD) prophylactic agent.

Embodiment 410. The multi-component cellular therapy product of embodiment 409, wherein the GVHD prophylactic agent is tacrolimus.

Embodiment 411. The multi-component cellular therapy product of embodiment 410, wherein the pharmaceutical composition comprises an amount of tacrolimus sufficient to maintain a trough blood level of approximately 5 ng/mL to approximately 15 ng/mL in a human subject receiving the product.

Embodiment 412. The multi-component cellular therapy product of embodiment 410 or embodiment 411, wherein the pharmaceutical composition comprises tacrolimus in an amount that ranges from approximately 0.01 mg per kilogram of body weight of a human subject receiving the product to approximately 0.50 mg per kilogram of body weight of a human subject receiving the product.

Embodiment 413. The multi-component cellular therapy product of any one of embodiments 410-412, wherein the pharmaceutical composition comprises tacrolimus an amount to maintain or that maintains a target blood level of approximately 1 ng/mL to approximately 10 ng/mL in a human subject receiving the product for approximately 20 days or more, approximately 25 days or more, approximately 30 days or more, approximately 35 days or more, approximately 40 days or more, approximately 55 days or more, approximately 50 days or more, approximately 55 days or more, approximately 60 days or more, approximately 65 days or more, approximately 770 days or more, approximately 75 days or more, approximately 80 days or more, approximately 85 days or more, approximately 90 days or more, approximately 95 days or more, approximately 100 days or more, approximately 110 days or more, approximately 120 days or more, approximately 130 days or more, approximately 140 days or more, or approximately 150 days after administration of the Tcons, after administration of the one or more cells comprising a chimeric receptor, or after administration of the Tcons and the one or more cells comprising a chimeric receptor.

Embodiment 414. The multi-component cellular therapy product of any one of embodiments 284-413, wherein the product comprises a dose of HSPCs that ranges from approximately 1.0×105 to approximately 1.0×108 HSPCs per kilogram of body weight of a human subject receiving the product, or from approximately 5.0×105 to approximately 1.5×1010 HSPCs.

Embodiment 415. The multi-component cellular therapy product of any one of embodiments 284-414, wherein the Tregs are FOXP3+.

Embodiment 416. The multi-component cellular therapy product of any one of embodiments 284-415, wherein the product comprises a dose of Tregs that ranges from approximately 1.0×105 to approximately 2.0×107 Tregs per kilogram of body weight of a human subject receiving the product, or from approximately 5.0×105 to approximately 3.0×109 Tregs.

Embodiment 417. The multi-component cellular therapy product of any one of embodiments 284-416, wherein the product comprises a dose of Tmems that ranges from approximately 1.0×105 to approximately 1.0×108 Tmems per kilogram of body weight of a human subject receiving the product, or from approximately 5.0×105 to approximately 1.5×1010 Tmems.

Embodiment 418. The multi-component cellular therapy product of any one of embodiments 284-417, wherein the product comprises a dose of Tcons that ranges from approximately 1.0×105 to approximately 4.0×107 Tcons per kilogram of body weight of a human subject receiving the product, or from approximately 5.0×105 to approximately 6.0×109 Tcons.

Embodiment 419. The multi-component cellular therapy product of any one of embodiments 284-418, wherein the one or more cells comprising a chimeric receptor are autologous with respect to a human subject receiving the product.

Embodiment 420. The multi-component cellular therapy product of any one of embodiments 284-418, wherein the one or more cells comprising a chimeric receptor are allogeneic with respect to a human subject receiving the product.

Embodiment 421. The multi-component cellular therapy product of embodiment 420, wherein the one or more cells comprising a chimeric receptor comprise one or more genetic modifications that increase immune tolerance with respect to a human subject receiving the product.

Embodiment 422. The multi-component cellular therapy product of embodiment 420, wherein the one or more cells comprising a chimeric receptor have not been modified to increase immune tolerance with respect to a human subject receiving the product.

Embodiment 423. The multi-component cellular therapy product of embodiment 422, wherein the one or more cells comprising a chimeric receptor are donor-derived from an allogeneic donor with respect to a human subject receiving the product.

Embodiment 424. The multi-component cellular therapy product of any one of embodiments 284-423, wherein the HSPCs, the Tregs, the Tmems, the Tcons, or any combination thereof are donor-isolated.

Embodiment 425. The multi-component cellular therapy product of embodiment 424, wherein the donor is allogeneic with respect to a human subject receiving the product.

Embodiment 426. The multi-component cellular therapy product of any one of embodiments 420-425, wherein the HSPCs, the Tregs, the Tmems, the Tcons, or any combination thereof, and the one or more cells comprising a chimeric receptor were derived from the same allogeneic donor.

Embodiment 427. The multi-component cellular therapy product of embodiment 425 or embodiment 426, wherein the allogeneic donor is unrelated to a human subject receiving the product.

Embodiment 428. The multi-component cellular therapy product of embodiment 425 or embodiment 426, wherein the allogeneic donor is related to a human subject receiving the product.

Embodiment 429. The multi-component cellular therapy product of any one of embodiments 420-428, wherein the allogeneic donor is HLA-matched, relative to a human subject receiving the product, at one or more alleles selected from the group consisting of: HLA-A, HLA-B, HLA-C, and HLA-DRB1.

Embodiment 430. The multi-component cellular therapy product of any one of embodiments 420-429, wherein the allogeneic donor is 8/8 HLA-matched, relative to a human subject receiving the product, for alleles HLA-A, HLA-B, HLA-C, and HLA-DRB1.

Embodiment 431. The multi-component cellular therapy product of any one of embodiments 420-428, wherein the allogeneic donor has at least one HLA mismatch, relative to a human subject receiving the product.

Embodiment 432. The multi-component cellular therapy product of embodiment 431, wherein the at least one HLA mismatch is at an allele selected from the group consisting of: HLA-A, HLA-B, HLA-C, HLA-DRB1, and any combination thereof.

Embodiment 433. The multi-component cellular therapy product of any one of embodiments 420-428, wherein the allogeneic donor is an allogeneic haploidentical donor, relative to a human subject receiving the product.

Embodiment 434. The multi-component cellular therapy product of embodiment 433, wherein the allogeneic haploidentical donor is at least 4/8 HLA-mismatched, at least 5/8 HLA-mismatched, or at least 6/8 HLA-mismatched at an allele selected from the group consisting of: HLA-A, HLA-B, HLA-C, HLA-DRB1, and any combination thereof.

Embodiment 435. The multi-component cellular therapy product of any one of embodiments 423-434, wherein the HSPCs, the Tregs, the Tmems, the Tcons, or any combination thereof, and/or the one or more cells comprising a chimeric receptor were obtained from one or more cord blood samples from the donor.

Embodiment 436. The multi-component cellular therapy product of any one of embodiments 423-434, wherein the HSPCs, the Tregs, the Tmems, the Tcons, or any combination thereof, and/or the one or more cells comprising a chimeric receptor were collected from one or more bone marrow samples from the donor.

Embodiment 437. The multi-component cellular therapy product of any one of embodiments 423-434, wherein the HSPCs, the Tregs, the Tmems, the Tcons, or any combination thereof, and/or the one or more cells comprising a chimeric receptor were collected from two or more peripheral blood samples from the donor.

Embodiment 438. The multi-component cellular therapy product of embodiment 436 or embodiment 437, wherein at least one of the two or more samples are mobilized samples.

Embodiment 439. The multi-component cellular therapy product of any one of embodiments 436-438, wherein at least one of the two or more samples are non-mobilized peripheral blood donations.

Embodiment 440. The multi-component cellular therapy product of any one of embodiments 436-439, wherein one or more non-mobilized samples are collected from the donor.

Embodiment 441. The multi-component cellular therapy product of embodiment 440, wherein one or more cells obtained from the one or more non-mobilized samples were used to produce the one or more cells comprising a chimeric receptor.

Embodiment 442. The multi-component cellular therapy product of any one of embodiments 436-441, wherein one or more mobilized samples are collected from the donor.

Embodiment 443. The multi-component cellular therapy product of embodiment 442, wherein two or more mobilized samples are collected from the donor.

Embodiment 444. The multi-component cellular therapy product of embodiment 442 or embodiment 443, wherein the HSPCs, the Tregs, the Tmems, the Tcons, or any combination thereof are obtainable from the mobilized samples.

Embodiment 445. The multi-component cellular therapy product of any one of embodiments 438-444, wherein at least one mobilized sample is mobilizable by granulocyte colony-stimulating factor (G-CSF), granulocyte macrophage colony-stimulating factor (GM-CSF), mozobil, or any combination thereof.

Embodiment 446. The multi-component cellular therapy product of any one of embodiments 438-445, wherein at least one mobilized sample is processed and sorted using one or more immune-separation particles (ISPs) to enrich for HSPCs, Tmems, Tregs, or any combination thereof.

Embodiment 447. The multi-component cellular therapy product of embodiment 446, wherein the one or more ISPs comprise affinity reagents, optionally wherein the affinity reagents are immuno-magnetic separation particles, optionally wherein the immuno-magnetic separation particles are antibodies each conjugated to an iron-containing particle.

Embodiment 448. The multi-component cellular therapy product of any one of embodiments 284-447, further comprising a conditioning regimen, wherein the conditioning regimen is formulated for administration before administration of the cellular therapy.

Embodiment 449. The multi-component cellular therapy product of embodiment 448, wherein the conditioning regimen is formulated for administration from approximately two days to approximately ten days before administration of the multi-component cellular therapy.

Embodiment 450. The multi-component cellular therapy product of embodiment 448 or embodiment 449, wherein the conditioning regimen is a total body irradiation-based (TBI-based) regimen or a total marrow and lymphoid irradiation-based (TMLI-based) regimen.

Embodiment 451. The multi-component cellular therapy product of embodiment 450, wherein the TBI-based regimen comprises fractionated total body irradiation (fTBI).

Embodiment 452. The multi-component cellular therapy product of embodiment 451, wherein the fTBI comprises 2 or more, 3 or more, 4 or more, 5 or more, 6 or more, 7 or more, 8 or more, 9 or more, or 10 or more doses.

Embodiment 453. The multi-component cellular therapy product of embodiment 451 or embodiment 452, wherein the fTBI comprises a total dose that ranges from approximately 500 to approximately 1600 cGy.

Embodiment 454. The multi-component cellular therapy product of any one of embodiments 451-453, wherein the fTBI comprises a total dose of approximately 1200 cGy, approximately 1210 cGy, approximately 1220, approximately 1240 cGy, approximately 1250 cGy approximately 1260 cGy, approximately 1270 cGy, approximately 1280 cGy, approximately 1290 cGy, approximately 1300 cGy, approximately 1310 cGy, approximately 1320 cGy, proximately 1340 cGy, approximately 1350 cGy approximately 1360 cGy, approximately 1370 cGy, approximately 1380 cGy, approximately 1390 cGy, approximately 1400 cGy, approximately 1410 cGy, approximately 1420, approximately 1440 cGy, approximately 1450 cGy approximately 1460 cGy, approximately 1470 cGy, approximately 1480 cGy, approximately 1490 cGy, or approximately 1500 cGy.

Embodiment 455. The multi-component cellular therapy product of any one of embodiments 451-454, wherein the fTBI-based regimen comprises 4 doses of TBI.

Embodiment 456. The multi-component cellular therapy product of any one of embodiments 450-455, wherein the TBI-based or TMLI-based regimen further comprises one or more doses of a conditioning reagent.

Embodiment 457. The multi-component cellular therapy product of embodiment 456, wherein the conditioning reagent comprises one or more conditioning reagents selected from the group consisting of: cyclophosphamide, etoposide, thiotepa, busulfan, melphalan, fludarabine, and any combination thereof.

Embodiment 458. The multi-component cellular therapy product of embodiment 456 or embodiment 457, wherein the TBI-based or TMLI-based regimen further comprises one or more doses of cyclophosphamide.

Embodiment 459. The multi-component cellular therapy product of embodiment 458, wherein each of the one or more doses of cyclophosphamide comprise from approximately 10 to approximately 100 mg of cyclophosphamide per kilogram of body weight of a human subject receiving the product.

Embodiment 460. The multi-component cellular therapy product of embodiment 458 or embodiment 459, wherein the one or more doses of cyclophosphamide comprise approximately 10 mg of cyclophosphamide per kilogram of body weight of a human subject receiving the product, approximately 15 mg of cyclophosphamide per kilogram of body weight of a human subject receiving the product, approximately 20 mg of cyclophosphamide per kilogram of body weight of a human subject receiving the product, approximately 25 mg of cyclophosphamide per kilogram of body weight of a human subject receiving the product, approximately 30 mg of cyclophosphamide per kilogram of body weight of a human subject receiving the product, approximately 35 mg of cyclophosphamide per kilogram of body weight of a human subject receiving the product, approximately 40 mg of cyclophosphamide per kilogram of body weight of a human subject receiving the product, approximately 45 mg of cyclophosphamide per kilogram of body weight of a human subject receiving the product, approximately 50 mg of cyclophosphamide per kilogram of body weight of a human subject receiving the product, approximately 55 mg of cyclophosphamide per kilogram of body weight of a human subject receiving the product, or approximately 60 mg of cyclophosphamide per kilogram of body weight of a human subject receiving the product.

Embodiment 461. The multi-component cellular therapy product of any one of embodiments 451-460, wherein the fTBI-based regimen further comprises two doses of cyclophosphamide.

Embodiment 462. The multi-component cellular therapy product of any one of embodiments 451-461, wherein a first dose of TBI or TMLI is formulated for administration approximately 8 days prior to administration of the HSPCs, prior to administration of the Tregs, prior to administration of the Tmems, or prior to administration of any combination of the HSPCs, the Tregs, and the Tmems.

Embodiment 463. The multi-component cellular therapy product of any one of embodiments 451-462, wherein a second dose of TBI is formulated for administration approximately 7 days prior to administration of the HSPCs, prior to administration of the Tregs, prior to administration of the Tmems, or prior to administration of any combination of the HSPCs, the Tregs, and the Tmems.

Embodiment 464. The multi-component cellular therapy product of any one of embodiments 451-463, wherein a third dose of TBI is formulated for administration approximately 6 days prior to administration of the HSPCs, prior to administration of the Tregs, prior to administration of the Tmems, or prior to administration of any combination of the HSPCs, the Tregs, and the Tmems.

Embodiment 465. The multi-component cellular therapy product of any one of embodiments 451-464, wherein a fourth dose of TBI is formulated for administration approximately 5 days prior to administration of the HSPCs, prior to administration of the Tregs, prior to administration of the Tmems, or prior to administration of any combination of the HSPCs, the Tregs, and the Tmems.

Embodiment 466. The multi-component cellular therapy product of any one of embodiments 451-465, wherein a first dose of cyclophosphamide is formulated for administration approximately 4 days prior to administration of the HSPCs, prior to administration of the Tregs, prior to administration of the Tmems, or prior to administration of any combination of the HSPCs, the Tregs, and the Tmems.

Embodiment 467. The multi-component cellular therapy product of any one of embodiments 451-466, wherein a second dose of cyclophosphamide is formulated for administration approximately 4 days prior to administration of the HSPCs, prior to administration of the Tregs, prior to administration of the Tmems, or prior to administration of any combination of the HSPCs, the Tregs, and the Tmems.

Embodiment 468. The multi-component cellular therapy product of embodiment 448 or embodiment 449, wherein the conditioning regimen is a myeloablative conditioning regimen or a reduced-intensity conditioning (RIC) regimen.

Embodiment 469. The multi-component cellular therapy product of embodiment 468, wherein the myeloablative conditioning regimen or RIC regimen comprises one or more conditioning reagents.

Embodiment 470. The multi-component cellular therapy product of embodiment 469, wherein the one or more conditioning reagents are selected from the group consisting of: thiotepa, busulfan, melphalan, fludarabine, cyclophosphamide, anti-thymocyte globulin (ATG), and any combination thereof.

Embodiment 471. The multi-component cellular therapy product of any one of embodiments 468-470, wherein the myeloablative conditioning regimen comprises three or more conditioning reagents, wherein at least one conditioning reagent comprises thiotepa.

Embodiment 472. The multi-component cellular therapy product of any one of embodiments 468-471, wherein the myeloablative conditioning regimen comprises one or more doses of busulfan, one or more doses of fludarabine, and one or more doses of thiotepa.

Embodiment 473. The multi-component cellular therapy product of embodiment 472, wherein each of the one or more doses of busulfan comprises from approximately 2 to approximately 11 mg of busulfan per kilogram of body weight of a human subject receiving the product.

Embodiment 474. The multi-component cellular therapy product of embodiment 472 or embodiment 473, wherein each of the one or more doses of busulfan comprises approximately 2.0 mg of busulfan per kilogram of body weight of a human subject receiving the product, approximately 2.1 mg of busulfan per kilogram of body weight of a human subject receiving the product, approximately 2.2 mg of busulfan per kilogram of body weight of a human subject receiving the product, approximately 2.3 mg of busulfan per kilogram of body weight of a human subject receiving the product, approximately 2.4 mg of busulfan per kilogram of body weight of a human subject receiving the product, approximately 2.5 mg of busulfan per kilogram of body weight of a human subject receiving the product, approximately 2.6 mg of busulfan per kilogram of body weight of a human subject receiving the product, approximately 2.7 mg of busulfan per kilogram of body weight of a human subject receiving the product, approximately 2.8 mg of busulfan per kilogram of body weight of a human subject receiving the product, approximately 2.9 mg of busulfan per kilogram of body weight of a human subject receiving the product, approximately 3.0 mg of busulfan per kilogram of body weight of a human subject receiving the product, approximately 3.1 mg of busulfan per kilogram of body weight of a human subject receiving the product, approximately 3.2 mg of busulfan per kilogram of body weight of a human subject receiving the product, approximately 3.3 mg of busulfan per kilogram of body weight of a human subject receiving the product, approximately 3.4 mg of busulfan per kilogram of body weight of a human subject receiving the product, approximately 3.5 mg of busulfan per kilogram of body weight of a human subject receiving the product, approximately 3.6 mg of busulfan per kilogram of body weight of a human subject receiving the product, approximately 3.7 mg of busulfan per kilogram of body weight of a human subject receiving the product, approximately 3.8 mg of busulfan per kilogram of body weight of a human subject receiving the product, approximately 3.9 mg of busulfan per kilogram of body weight of a human subject receiving the product, approximately 4.0 mg of busulfan per kilogram of body weight of a human subject receiving the product, approximately 4.1 mg of busulfan per kilogram of body weight of a human subject receiving the product, approximately 4.2 mg of busulfan per kilogram of body weight of a human subject receiving the product, approximately 4.3 mg of busulfan per kilogram of body weight of a human subject receiving the product, approximately 4.4 mg of busulfan per kilogram of body weight of a human subject receiving the product, approximately 4.5 mg of busulfan per kilogram of body weight of a human subject receiving the product, approximately 4.6 mg of busulfan per kilogram of body weight of a human subject receiving the product, approximately 4.7 mg of busulfan per kilogram of body weight of a human subject receiving the product, approximately 4.8 mg of busulfan per kilogram of body weight of a human subject receiving the product, approximately 4.9 mg of busulfan per kilogram of body weight of a human subject receiving the product, approximately 5.0 mg of busulfan per kilogram of body weight of a human subject receiving the product, approximately 5.1 mg of busulfan per kilogram of body weight of a human subject receiving the product, approximately 5.2 mg of busulfan per kilogram of body weight of a human subject receiving the product, approximately 5.3 mg of busulfan per kilogram of body weight of a human subject receiving the product, approximately 5.4 mg of busulfan per kilogram of body weight of a human subject receiving the product, approximately 5.5 mg of busulfan per kilogram of body weight of a human subject receiving the product, approximately 5.6 mg of busulfan per kilogram of body weight of a human subject receiving the product, approximately 5.7 mg of busulfan per kilogram of body weight of a human subject receiving the product, approximately 5.8 mg of busulfan per kilogram of body weight of a human subject receiving the product, approximately 5.9 mg of busulfan per kilogram of body weight of a human subject receiving the product, or approximately 6.0 mg of busulfan per kilogram of body weight of a human subject receiving the product.

Embodiment 475. The multi-component cellular therapy product of any one of embodiments 472-474, wherein the myeloablative conditioning regimen comprises three doses of busulfan.

Embodiment 476. The multi-component cellular therapy product of any one of embodiments 472-475, wherein each of the one or more doses of fludarabine comprises from approximately 30 to approximately 200 mg of fludarabine per meter2 body surface area.

Embodiment 477. The multi-component cellular therapy product of any one of embodiments 472-476, wherein each of the one or more doses of fludarabine comprises 40 mg of fludarabine per meter2 body surface area, 41 mg of fludarabine per meter2 body surface area, 42 mg of fludarabine per meter2 body surface area, 43 mg of fludarabine per meter2 body surface area, 44 mg of fludarabine per meter2 body surface area, 44 mg of fludarabine per meter2 body surface area, 46 mg of fludarabine per meter2 body surface area, 47 mg of fludarabine per meter2 body surface area, 48 mg of fludarabine per meter2 body surface area, 49 mg of fludarabine per meter2 body surface area, approximately 50 mg of fludarabine per meter2 body surface area, 51 mg of fludarabine per meter2 body surface area, 52 mg of fludarabine per meter2 body surface area, 53 mg of fludarabine per meter2 body surface area, 54 mg of fludarabine per meter2 body surface area, 55 mg of fludarabine per meter2 body surface area, 56 mg of fludarabine per meter2 body surface area, 57 mg of fludarabine per meter2 body surface area, 58 mg of fludarabine per meter2 body surface area, 59 mg of fludarabine per meter2 body surface area, approximately 60 mg of fludarabine per meter2 body surface area, 61 mg of fludarabine per meter2 body surface area, 62 mg of fludarabine per meter2 body surface area, 63 mg of fludarabine per meter2 body surface area, 64 mg of fludarabine per meter2 body surface area, 65 mg of fludarabine per meter2 body surface area, 66 mg of fludarabine per meter2 body surface area, 67 mg of fludarabine per meter2 body surface area, 68 mg of fludarabine per meter2 body surface area, 69 mg of fludarabine per meter2 body surface area, approximately 70 mg of fludarabine per meter2 body surface area, 71 mg of fludarabine per meter2 body surface area, 72 mg of fludarabine per meter2 body surface area, 73 mg of fludarabine per meter2 body surface area, 74 mg of fludarabine per meter2 body surface area, or 75 mg of fludarabine per meter2 body surface area.

Embodiment 478. The multi-component cellular therapy product of embodiment 476 or embodiment 477, wherein the myeloablative conditioning regimen comprises three doses of fludarabine.

Embodiment 479. The multi-component cellular therapy product of any one of embodiments 472-477, wherein each of the one or more doses of thiotepa comprises from approximately 3 to approximately 12 mg of thiotepa per kilogram of body weight of a human subject receiving the product.

Embodiment 480. The multi-component cellular therapy product of any one of embodiments 472-479, wherein each of the one or more doses of thiotepa comprises approximately 3.0 mg of thiotepa per kilogram of body weight of a human subject receiving the product, 3.1 mg of thiotepa per kilogram of body weight of a human subject receiving the product, 3.2 mg of thiotepa per kilogram of body weight of a human subject receiving the product, 3.3 mg of thiotepa per kilogram of body weight of a human subject receiving the product, 3.4 mg of thiotepa per kilogram of body weight of a human subject receiving the product, 3.5 mg of thiotepa per kilogram of body weight of a human subject receiving the product, 3.6 mg of thiotepa per kilogram of body weight of a human subject receiving the product, 3.7 mg of thiotepa per kilogram of body weight of a human subject receiving the product, 3.8 mg of thiotepa per kilogram of body weight of a human subject receiving the product, 3.9 mg of thiotepa per kilogram of body weight of a human subject receiving the product, approximately 4.0 mg of thiotepa per kilogram of body weight of a human subject receiving the product, 4.1 mg of thiotepa per kilogram of body weight of a human subject receiving the product, 4.2 mg of thiotepa per kilogram of body weight of a human subject receiving the product, 4.3 mg of thiotepa per kilogram of body weight of a human subject receiving the product, 4.4 mg of thiotepa per kilogram of body weight of a human subject receiving the product, 4.5 mg of thiotepa per kilogram of body weight of a human subject receiving the product, 4.6 mg of thiotepa per kilogram of body weight of a human subject receiving the product, 4.7 mg of thiotepa per kilogram of body weight of a human subject receiving the product, 4.8 mg of thiotepa per kilogram of body weight of a human subject receiving the product, 4.9 mg of thiotepa per kilogram of body weight of a human subject receiving the product, approximately 5.0 mg of thiotepa per kilogram of body weight of a human subject receiving the product, 5.1 mg of thiotepa per kilogram of body weight of a human subject receiving the product, 5.2 mg of thiotepa per kilogram of body weight of a human subject receiving the product, 5.3 mg of thiotepa per kilogram of body weight of a human subject receiving the product, 5.4 mg of thiotepa per kilogram of body weight of a human subject receiving the product, 5.5 mg of thiotepa per kilogram of body weight of a human subject receiving the product, 5.6 mg of thiotepa per kilogram of body weight of a human subject receiving the product, 5.7 mg of thiotepa per kilogram of body weight of a human subject receiving the product, 5.8 mg of thiotepa per kilogram of body weight of a human subject receiving the product, 5.9 mg of thiotepa per kilogram of body weight of a human subject receiving the product, approximately 6.0 mg of thiotepa per kilogram of body weight of a human subject receiving the product, 6.1 mg of thiotepa per kilogram of body weight of a human subject receiving the product, 6.2 mg of thiotepa per kilogram of body weight of a human subject receiving the product, 6.3 mg of thiotepa per kilogram of body weight of a human subject receiving the product, 6.4 mg of thiotepa per kilogram of body weight of a human subject receiving the product, 6.5 mg of thiotepa per kilogram of body weight of a human subject receiving the product, 6.6 mg of thiotepa per kilogram of body weight of a human subject receiving the product, 6.7 mg of thiotepa per kilogram of body weight of a human subject receiving the product, 6.8 mg of thiotepa per kilogram of body weight of a human subject receiving the product, 6.9 mg of thiotepa per kilogram of body weight of a human subject receiving the product, or 7.0 mg of thiotepa per kilogram of body weight of a human subject receiving the product.

Embodiment 481. The multi-component cellular therapy product of embodiment 479 or embodiment 480, wherein the myeloablative conditioning regimen comprises two doses of thiotepa.

Embodiment 482. The multi-component cellular therapy product of any one of embodiments 472-481, wherein a first dose of busulfan and a first dose of fludarabine are formulated for administration approximately 6 days prior to administration of the HSPCs, prior to administration of the Tregs, prior to administration of the Tmems, or prior to administration of any combination of HSPCs, Tregs, and Tmems.

Embodiment 483. The multi-component cellular therapy product of any one of embodiments 472-482, wherein a second dose of busulfan and a second dose of fludarabine are formulated for administration approximately 5 days prior to administration of the HSPCs, prior to administration of the Tregs, prior to administration of the Tmems, or prior to administration of any combination of HSPCs, Tregs, and Tmems.

Embodiment 484. The multi-component cellular therapy product of any one of embodiments 472-483, wherein a third dose of busulfan and a third dose of fludarabine are formulated for administration approximately 4 days prior to administration of the HSPCs, prior to administration of the Tregs, prior to administration of the Tmems, or prior to administration of any combination of HSPCs, Tregs, and Tmems.

Embodiment 485. The multi-component cellular therapy product of any one of embodiments 472-484, wherein a first dose of thiotepa is formulated for administration approximately 3 days prior to administration of the HSPCs, prior to administration of the Tregs, prior to administration of the Tmems, or prior to administration of any combination of HSPCs, Tregs, and Tmems.

Embodiment 486. The multi-component cellular therapy product of any one of embodiments 472-485, wherein a second dose of thiotepa is formulated for administration approximately 2 days prior to administration of the HSPCs, prior to administration of the Tregs, prior to administration of the Tmems, or prior to administration of any combination of HSPCs, Tregs, and Tmems.

Embodiment 487. The multi-component cellular therapy product of any one of embodiments 284-486, wherein the chimeric receptor comprises one or more extracellular antigen-binding domains that bind to one or more antigens selected from the group consisting of: BCMA, PSMA, CD8, CD10, CD19, CD20, CD21, CD22, CD25, CD30, CD33, CD34, CD37, CD44v6, CD45, CD52, CSF1R (CD115), c-Kit (CD117), CD123, CD133, Fms-like tyrosine kinase 3 (FLT-3, CD135), PDGFR-alpha (CD140a), PDGFR-beta (CD140b), CD186, CD195, VEGFR3 (FLT4, CD309), GPRC5D, TMEFF2, ROR1, B7-H6, B7-H3, HM1.24, SLAMF7, chondroitin sulfate proteoglycan 4 (CSPG4, melanoma-associated chondroitin sulfate proteoglycan), epidermal growth factor receptor (EGFR), Her2, Her3, IGFR, IL3R, fibroblast activating protein (FAP), CDCP1, Derlin1, Tenascin, frizzled 1-10, VEGFR2 (KDR/FLK1), endoglin, CLEC14, Tem1-8, Tie2, A33, CAMPATH-1 (CDw52), CEA, Carbonic anhydrase IX (MN/CA IX), de2-7, EGFRvIII, EpCAM, Ep-CAM, folate-binding protein, G250, HLA-DR, IGFR, IL-2 receptor, IL3R, MCSP (melanoma-associated cell surface chondroitin sulphate proteoglycane), Muc-1, prostate stem cell antigen (PSCA), prostate specific antigen (PSA), hK2, TAG-72, KI2L4, NKG2E, NKG2D, NKG2F, BTNL3, BTNL8, PD-1, NKG2, a tumor cell neoantigen, and any combination thereof.

Embodiment 488. The multi-component cellular therapy product of embodiment 487, wherein the chimeric receptor is a chimeric antigen receptor (CAR) or T cell receptor (TCR).

Embodiment 489. The multi-component cellular therapy product of embodiment 488, wherein the CAR further comprises a transmembrane domain and one or more intracellular signaling domains.

Embodiment 490. The multi-component cellular therapy product of embodiment 489, wherein the transmembrane domain is selected from the group consisting of: a CD8 transmembrane domain, a CD28 transmembrane domain a CD3zeta-chain transmembrane domain, a CD4 transmembrane domain, a 4-IBB transmembrane domain, an 0X40 transmembrane domain, an ICOS transmembrane domain, a CTLA-4 transmembrane domain, aPD-1 transmembrane domain, a LAG-3 transmembrane domain, a 2B4 transmembrane domain, a BTLA transmembrane domain, an 0X40 transmembrane domain, a DAP 10 transmembrane domain, a DAP 12 transmembrane domain, a CD16a transmembrane domain, a DNAM-1 transmembrane domain, a KIR2DSl transmembrane domain, aKIR3DSl transmembrane domain, an NKp44 transmembrane domain, an NKp46 transmembrane domain, an FceRlg transmembrane domain, and an NKG2D transmembrane domain.

Embodiment 491. The multi-component cellular therapy product of embodiment 489 or embodiment 490, wherein the one or more intracellular signaling domains are each selected from the group consisting of: a CD3zeta-chain intracellular signaling domain, a CD97 intracellular signaling domain, a CD1 la-CD18 intracellular signaling domain, a CD2 intracellular signaling domain, an ICOS intracellular signaling domain, a CD27 intracellular signaling domain, a CD154 intracellular signaling domain, a CDS intracellular signaling domain, an 0X40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP 10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceRlg intracellular signaling domain, a NKG2D intracellular signaling domain, and an EAT-2 intracellular signaling domain.

Embodiment 492. The multi-component cellular therapy product of any one of embodiments 488-491, wherein the CAR is a bispecific CAR that comprises two or more extracellular antigen-binding domains.

Embodiment 493. The multi-component cellular therapy product of any one of embodiments 488-492, wherein the CAR comprises a first antigen-binding domain specific for CD22 and a second antigen-binding domain specific for CD19.

Embodiment 494. The multi-component cellular therapy product of embodiment 493, wherein the first antigen-binding domain comprises a heavy chain variable (VH) region and a light chain variable (VL) region,
wherein the VH comprises: a heavy chain complementarity determining region 1 (CDR-H1) having the amino acid sequence of GDSVSSNSAA (SEQ ID NO: 1), a heavy chain complementarity determining region 2 (CDR-H2) having the amino acid sequence of TYYRSKWYN (SEQ ID NO: 2), and a heavy chain complementarity determining region 3 (CDR-H3) having the amino acid sequence of AREVTGDLEDAFDI (SEQ ID NO: 3), and
wherein the VL comprises: a light chain complementarity determining region 1 (CDR-L1) having the amino acid sequence of QTIWSY (SEQ ID NO: 4), a light chain complementarity determining region 2 (CDR-L2) having the amino acid sequence of AAS (SEQ ID NO: 5), and a light chain complementarity determining region 3 (CDR-L3) having the amino acid sequence of QQSYSIPQT (SEQ ID NO: 6).

Embodiment 495. The multi-component cellular therapy product of embodiment 493 or embodiment 494, wherein the second antigen-binding domain comprises a heavy chain variable (VH) region and a light chain variable (VL) region,
wherein the VH comprises: a heavy chain complementarity determining region 1 (CDR-H1) having the amino acid sequence of GVSLPDYG (SEQ ID NO: 7), a heavy chain complementarity determining region 2 (CDR-H2) having the amino acid sequence of IWGSETT (SEQ ID NO: 8), and a heavy chain complementarity determining region 3 (CDR-H3) having the amino acid sequence of AKHYYYGGSYAMDY (SEQ ID NO: 9), and
wherein the VL comprises: a light chain complementarity determining region 1 (CDR-L1) having the amino acid sequence of QDISKY (SEQ ID NO: 10), a light chain complementarity determining region 2 (CDR-L2) having the amino acid sequence of HTS (SEQ ID NO: 11), and a light chain complementarity determining region 3 (CDR-L3) having the amino acid sequence of QQGNTLPYT (SEQ ID NO: 12).

Embodiment 496. The multi-component cellular therapy product of any one of embodiments 493-495 wherein the first antigen-binding domain comprises a VH having amino acid sequence of

(SEQ ID NO: 13)
QVQLQQSGPGLVKPSQTLSLTCAISGDSVSSNSAAWNWIRQSPSRGLEW
LGRTYYRSKWYNDYAVSVKSRITINPDTSKNQFSLQLNSVTPEDTAVYY
CAREVTGDLEDAFDIWGQGTMVTVSS.

Embodiment 497. The multi-component cellular therapy product of any one of embodiments 493-496, wherein the first antigen-binding domain comprises a VL having amino acid sequence of

(SEQ ID NO: 14)
DIQMTQSPSSLSASVGDRVTITCRASQTIWSYLNWYQQRPGKAPNLLIY
AASSLQSGVPSRFSGRGSGTDFTLTISSLQAEDFATYYCQQSYSIPQTF
GQGTKLEIK.

Embodiment 498. The multi-component cellular therapy product of any one of embodiments 493-497, wherein the second antigen-binding domain comprises a VH having amino acid sequence of

(SEQ ID NO: 15)
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSS.

Embodiment 499. The multi-component cellular therapy product of any one of embodiments 493-498, wherein the second antigen-binding domain comprises a VL having the amino acid sequence of

(SEQ ID NO: 16)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEIT.

Embodiment 500. The multi-component cellular therapy product of any one of embodiments 493-499, wherein the first antigen-binding domain comprises a CD22 scFv having the amino acid sequence of:

(a)
(SEQ ID NO: 20)
QVQLQQSGPGLVKPSQTLSLTCAISGDSVSSNSAAWNWIRQSPSRGLEWLGRTYYRSKWYND
YAVSVKSRITINPDTSKNQFSLQLNSVTPEDTAVYYCAREVTGDLEDAFDIWGQGTMVTVSSGST
SGSGKPGSGEGSTKGDIQMTQSPSSLSASVGDRVTITCRASQTIWSYLNWYQQRPGKAPNLLIYA
ASSLQSGVPSRFSGRGSGTDFTLTISSLQAEDFATYYCQQSYSIPQTFGQGTKLEIK;
or
(b)
(SEQ ID NO: 21)
QVQLQQSGPGLVKPSQTLSLTCAISGDSVSSNSAAWNWIRQSPSRGLEWLGRTYYRSKWYND
YAVSVKSRITINPDTSKNQFSLQLNSVTPEDTAVYYCAREVTGDLEDAFDIWGQGTMVTVSSGG
GGSDIQMTQSPSSLSASVGDRVTITCRASQTIWSYLNWYQQRPGKAPNLLIYAASSLQSGVPSRFS
GRGSGTDFTLTISSLQAEDFATYYCQQSYSIPQTFGQGTKLEIK.

Embodiment 501. The multi-component cellular therapy product of any one of embodiment 493-500, wherein the second antigen-binding domain comprises a CD19 scFv having the amino acid sequence of:

(a)
(SEQ ID NO: 22)
DIQMTQTTSSLSASLGDRVTISCRASQDISKYLNWYQQKPDGTVKLLIYHTSRLHSGVPSRFSG
SGSGTDYSLTISNLEQEDIATYFCQQGNTLPYTFGGGTKLEITGSTSGSGKPGSGEGSTKGEVKLQ
ESGPGLVAPSQSLSVTCTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSS;
or
(b)
(SEQ ID NO: 41)
DIQMTQTTSSLSASLGDRVTISCRASQDISKYLNWYQQKPDGTVKLLIYHTSRLHSGVPSRFSG
SGSGTDYSLTISNLEQEDIATYFCQQGNTLPYTFGGGTKLEITGGGGSEVKLQESGPGLVAPSQSL
SVTCTVSGVSLPDYGVSWIRQPPRKGLEWLGVIWGSETTYYNSALKSRLTIIKDNSKSQVFLKMN
SLQTDDTAIYYCAKHYYYGGSYAMDYWGQGTSVTVSS.

Embodiment 502. The multi-component cellular therapy product of any one of embodiments 493-501, wherein the CAR comprises the amino acid sequence of:

(a)
(SEQ ID NO: 23)
DIQMTQTTSSLSASLGDRVTISCRASQDISKYLNWYQQKPDGTVKLLIYHTSRLHSGVPSRFSG
SGSGTDYSLTISNLEQEDIATYFCQQGNTLPYTFGGGTKLEITGGGGSQVQLQQSGPGLVKPSQTL
SLTCAISGDSVSSNSAAWNWIRQSPSRGLEWLGRTYYRSKWYNDYAVSVKSRITINPDTSKNQFS
LQLNSVTPEDTAVYYCAREVTGDLEDAFDIWGQGTMVTVSSGSTSGSGKPGSGEGSTKGDIQMT
QSPSSLSASVGDRVTITCRASQTIWSYLNWYQQRPGKAPNLLIYAASSLQSGVPSRFSGRGSGTDF
TLTISSLQAEDFATYYCQQSYSIPQTFGQGTKLEIKGGGGSEVKLQESGPGLVAPSQSLSVTCTVS
GVSLPDYGVSWIRQPPRKGLEWLGVIWGSETTYYNSALKSRLTIIKDNSKSQVFLKMNSLQTDDT
AIYYCAKHYYYGGSYAMDYWGQGTSVTVSSS;
(b)
(SEQ ID NO: 24)
QVQLQQSGPGLVKPSQTLSLTCAISGDSVSSNSAAWNWIRQSPSRGLEWLGRTYYRSKWYND
YAVSVKSRITINPDTSKNQFSLQLNSVTPEDTAVYYCAREVTGDLEDAFDIWGQGTMVTVSSGG
GGSDIQMTQSPSSLSASVGDRVTITCRASQTIWSYLNWYQQRPGKAPNLLIYAASSLQSGVPSRFS
GRGSGTDFTLTISSLQAEDFATYYCQQSYSIPQTFGQGTKLEIKGGGGSGGGGSGGGGSGGGGSG
GGGSDIQMTQTTSSLSASLGDRVTISCRASQDISKYLNWYQQKPDGTVKLLIYHTSRLHSGVPSR
FSGSGSGTDYSLTISNLEQEDIATYFCQQGNTLPYTFGGGTKLEITGSTSGSGKPGSGEGSTKGEV
KLQESGPGLVAPSQSLSVTCTVSGVSLPDYGVSWIRQPPRKGLEWLGVIWGSETTYYNSALKSRL
TIIKDNSKSQVFLKMNSLQTDDTAIYYCAKHYYYGGSYAMDYWGQGTSVTVSS;
(c)
(SEQ ID NO: 29)
DIQMTQTTSSLSASLGDRVTISCRASQDISKYLNWYQQKPDGTVKLLIYHTSRLHSGVPSRFSG
SGSGTDYSLTISNLEQEDIATYFCQQGNTLPYTFGGGTKLEITGGGGSQVQLQQSGPGLVKPSQTL
SLTCAISGDSVSSNSAAWNWIRQSPSRGLEWLGRTYYRSKWYNDYAVSVKSRITINPDTSKNQFS
LQLNSVTPEDTAVYYCAREVTGDLEDAFDIWGQGTMVTVSSGSTSGSGKPGSGEGSTKGDIQMT
QSPSSLSASVGDRVTITCRASQTIWSYLNWYQQRPGKAPNLLIYAASSLQSGVPSRFSGRGSGTDF
TLTISSLQAEDFATYYCQQSYSIPQTFGQGTKLEIKGGGGSEVKLQESGPGLVAPSQSLSVTCTVS
GVSLPDYGVSWIRQPPRKGLEWLGVIWGSETTYYNSALKSRLTIIKDNSKSQVFLKMNSLQTDDT
AIYYCAKHYYYGGSYAMDYWGQGTSVTVSSSGTTTPAPRPPTPAPTIASQPLSLRPEACRPAAGG
AVHTRGLDFACDIYIWAPLAGTCGVLLLSLVITLYCKRGRKKLLYIFKQPFMRPVQTTQEEDGCS
CRFPEEEEGGCELRVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDTYDALHMQALPPR;
or
(d)
(SEQ ID NO: 30)
QVQLQQSGPGLVKPSQTLSLTCAISGDSVSSNSAAWNWIRQSPSRGLEWLGRTYYRSKWYND
YAVSVKSRITINPDTSKNQFSLQLNSVTPEDTAVYYCAREVTGDLEDAFDIWGQGTMVTVSSGG
GGSDIQMTQSPSSLSASVGDRVTITCRASQTIWSYLNWYQQRPGKAPNLLIYAASSLQSGVPSRFS
GRGSGTDFTLTISSLQAEDFATYYCQQSYSIPQTFGQGTKLEIKGGGGSGGGGSGGGGSGGGGSG
GGGSDIQMTQTTSSLSASLGDRVTISCRASQDISKYLNWYQQKPDGTVKLLIYHTSRLHSGVPSR
FSGSGSGTDYSLTISNLEQEDIATYFCQQGNTLPYTFGGGTKLEITGSTSGSGKPGSGEGSTKGEV
KLQESGPGLVAPSQSLSVTCTVSGVSLPDYGVSWIRQPPRKGLEWLGVIWGSETTYYNSALKSRL
TIIKDNSKSQVFLKMNSLQTDDTAIYYCAKHYYYGGSYAMDYWGQGTSVTVSSTTTPAPRPPTP
APTIASQPLSLRPEACRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYKQGQNQLYNELNLGRR
EEYDVLDKRRGRDPEMGGKPRRKNPQEGLYNELQKDKMAEAYSEIGMKGERRRGKGHDGLYQ
GLSTATKDTYDALHMQALPPR.

Embodiment 503. The multi-component cellular therapy product of any one of embodiments 488-491, wherein the CAR comprises an antigen-binding domain specific for B-cell maturation antigen (BCMA).

Embodiment 504. The multi-component cellular therapy product of embodiment 503, wherein the antigen-binding domain comprises a heavy chain variable (VH) region comprising a heavy chain complementarity determining region 1 (CDR-H1) a complementarity determining region 2 (CDR-H2), and a complementarity determining region 3 (CDR-H3),
wherein the VH comprises CDR-H1, CDR-H2, and CDR-H3 amino acid sequences selected from the group consisting of:

a)
(SEQ ID NO: 42)
GFTFTNHA,
(SEQ ID NO: 43)
ISGNGRTT,
and
(SEQ ID NO: 44)
AKDGGETLVDS;
b)
(SEQ ID NO: 45)
GFTFSSHA;
(SEQ ID NO: 46)
ISGSGDFT,
and
(SEQ ID NO: 47)
AKDEDGGSLLGY;
c)
(SEQ ID NO: 48)
GFTFSSYA;
(SEQ ID NO: 49)
ISGSGDYI,
and
(SEQ ID NO: 50)
AKEGTGANSSLADY;
and
d)
(SEQ ID NO: 51)
GFTFSSHA,
(SEQ ID NO: 52)
ISGSGDYT,
and
(SEQ ID NO: 53)
AKDEDGGSLLGH.

Embodiment 505. The multi-component cellular therapy product of embodiment 503 or embodiment 504, wherein the antigen-binding domain comprises a VH having amino acid sequence selected from the group consisting of:

a)
(SEQ ID NO: 54)
QVQLVESGGGLVQPGGSLRLSCAASGFTFTNHAMSWVRQAPGKGLELVS
SISGNGRTTYYADSVKGRFTISRDISKNTLDLQMNSLRAEDTAVYYCAK
DGGETLVDSRGQGTLVTVSS;
b)
(SEQ ID NO: 55)
QVQLVESGGGLVQPGGSLRLSCAASGFTFSSHAMTWVRQAPGKGLEWVA
AISGSGDFTHYADSVKGRFTISRDNSKNTVSLQMNNLRAEDTAVYYCAK
DEDGGSLLGYRGQGTLVTVSS;
c)
(SEQ ID NO: 56)
EVQLLESGGGLVQPGGSLRLSCAASGFTFSSYAMSWVRQAPGKGLEWVS
SISGSGDYIYYADSVKGRFTISRDISKNTLYLQMNSLRAEDTAVYYCAK
EGTGANSSLADYRGQGTLVTVSS;
and
d)
(SEQ ID NO: 57)
EVQLLESGGGLIQPGGSLRLSCAASGFTFSSHAMTWVRQAPGKGLEWVS
AISGSGDYTHYADSVKGRFTISRDNSKNTVYLQMNSLRAEDSAVYYCAK
DEDGGSLLGHRGQGTLVTVSS.

Embodiment 506. The multi-component cellular therapy product of embodiment 503, wherein the antigen-binding domain comprises a heavy chain variable (VH) region and a light chain variable (VL) region,
wherein the VH comprises an amino acid sequence selected from the group consisting of:

a)
(SEQ ID NO: 58)
QIQLVQSGPELKKPGETVKISCKASGYTFRHYSMNWVKQAPGKGLKWMG
RINTESGVPIYADDFKGRFAFSVETSASTAYLVINNLKDEDTASYFCSN
DYLYSLDFWGQGTALTVSS;
and
b)
(SEQ ID NO: 60)
QIQLVQSGPELKKPGETVKISCKASGYTFTDYSINWVKRAPGKGLKWMG
WINTETREPAYAYDFRGRFAFSLETSASTAYLQINNLKYEDTATYFCAL
DYSYAMDYWGQGTSVTVSS,

and
wherein the VL comprises an amino acid sequence selected from the group consisting of:

a)
(SEQ ID NO: 59)
DIVLTQSPPSLAMSLGKRATISCRASESVTILGSHLIYWYQQKPGQPPT
LLIQLASNVQTGVPARFSGSGSRTDFTLTIDPVEEDDVAVYYCLQSRTI
PRTFGGGTKLEIK;
b)
(SEQ ID NO: 61)
DIVLTQSPPSLAMSLGKRATISCRASESVTILGSHLIHWYQQKPGQPP
TLLIQLASNVQTGVPARFSGSGSRTDFTLTIDPVEEDDVAVYYCLQSRT
IPRTFGGGTKLEIK;
c)
(SEQ ID NO: 62)
DIVLTQSPASLAVSLGQRATISCRASESVDNYGFSFMHWYQQKPGQPPK
LLIYRASNLESGIPARFSGSGSRTDFALTINPVETDDVATYYCQQSNKD
PRTFGGGTKLEIK;
and
d)
(SEQ ID NO: 63)
DIVLTQSPASLAVSLGQRATISCRASESVDNYGFSFMHWYQQKPGQPPK
LLIYRASNLESGIPARFSGSGSRTDFALTINPVETDDVATYYCQQSNKD
PRTFGGGTKLEIK.

Embodiment 507. The multi-component cellular therapy product of embodiment 506, wherein the antigen-binding domain comprises a BCMA scFv having an amino acid sequence selected from the group consisting of:

a)
(SEQ ID NO: 64)
DIVLTQSPPSLAMSLGKRATISCRASESVTILGSHLIYWYQQKPGQPPTLLIQLASNVQTGVPAR
FSGSGSRTDFTLTIDPVEEDDVAVYYCLQSRTIPRTFGGGTKLEIKGSTSGSGKPGSGEGSTKGQIQ
LVQSGPELKKPGETVKISCKASGYTFRHYSMNWVKQAPGKGLKWMGRINTESGVPIYADDFKG
RFAFSVETSASTAYLVINNLKDEDTASYFCSNDYLYSLDFWGQGTALTVSS;
b)
(SEQ ID NO: 65)
DIVLTQSPPSLAMSLGKRATISCRASESVTILGSHLIHWYQQKPGQPPTLLIQLASNVQTGVPAR
FSGSGSRTDFTLTIDPVEEDDVAVYYCLQSRTIPRTFGGGTKLEIKGSTSGSGKPGSGEGSTKGQIQ
LVQSGPELKKPGETVKISCKASGYTFTDYSINWVKRAPGKGLKWMGWINTETREPAYAYDFRG
RFAFSLETSASTAYLQINNLKYEDTATYFCALDYSYAMDYWGQGTSVTVSS;
c)
(SEQ ID NO: 66)
DIVLTQSPASLAVSLGQRATISCRASESVDNYGFSFMHWYQQKPGQPPKLLIYRASNLESGIPAR
FSGSGSRTDFALTINPVETDDVATYYCQQSNKDPRTFGGGTKLEIKGSTSGSGKPGSGEGSTKGQI
QLVQSGPELKKPGETVKISCKASGYTFTDYSINWVKRAPGKGLKWMGWINTETREPAYAYDFR
GRFAFSLETSASTAYLQINNLKYEDTATYFCALDYSYAMDYWGQGTSVTVSS;
and
d)
(SEQ ID NO: 67)
DIVLTQSPASLAMSLGKRATISCRASESVSVIGAHLIHWYQQKPGQPPKLLIYLASNLETGVPAR
FSGSGSGTDFTLTIDPVEEDDVAIYSCLQSRIFPRTFGGGTKLEIKGSTSGSGKPGSGEGSTKGQIQL
VQSGPELKKPGETVKISCKASGYTFTDYSINWVKRAPGKGLKWMGWINTETREPAYAYDFRGR
FAFSLETSASTAYLQINNLKYEDTATYFCALDYSYAMDYWGQGTSVTVSS.

Embodiment 508. The multi-component cellular therapy product of any one of embodiments 488-491, wherein the CAR comprises one or more single-domain binding regions specific for B-cell maturation antigen (BCMA), wherein each single-domain binding region comprises an amino acid sequence selected from the group consisting of:

a)
(SEQ ID NO: 68)
QVKLEESGGGLVQAGRSLRLSCAASEHTFSSHVMGWFRQAPGKERESV
AVIGWRDISTSYADSVKGRFTISRDNAKKTLYLQMNSLKPEDTAVYYC
AARRIDAADFDSWGQGTQVTVSS;
and
b)
(SEQ ID NO: 69)
EVQLVESGGGLVQAGGSLRLSCAASGRTFTMGWFRQAPGKEREFVAAI
SLSPTLAYYAESVKGRFTISRDNAKNTVVLQMNSLKPEDTALYYCAAD
RKSVMSIRPDYWGQGTQVTVSS.

Embodiment 509. The multi-component cellular therapy product of any one of embodiments 488-491, wherein the CAR comprises an antigen-binding domain specific for B-cell maturation antigen (BCMA), wherein the binding domain comprises the amino acid sequence of

(SEQ ID NO: 70)
MGSWSEFWVRLGAIRERLDALGGSEAELAAFEKEIAAFESELQAYKGKG
NPEVEKLRYTAATIRRFLQAYRHN.

Embodiment 510. The multi-component cellular therapy product of any one of embodiments 503-509, wherein the CAR comprises an amino acid sequence selected from the group consisting of:

a)
(SEQ ID NO: 71)
MALPVTALLLPLALLLHAARPQVQLVESGGGLVQPGGSLRLSCAASGFTFTNHAMSWVRQAP
GKGLELVSSISGNGRTTYYADSVKGRFTISRDISKNTLDLQMNSLRAEDTAVYYCAKDGGETLV
DSRGQGTLVTVSSFVPVFLPAKPTTTPAPRPPTPAPTIASQPLSLRPEACRPAAGGAVHTRGLDFA
CDIYIWAPLAGTCGVLLLSLVITLYCNHRNRSKRSRLLHSDYMNMTPRRPGPTRKHYQPYAPPR
DFAAYRSRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR;
b)
(SEQ ID NO: 72)
MALPVTALLLPLALLLHAARPQVQLVESGGGLVQPGGSLRLSCAASGFTFSSHAMTWVRQAP
GKGLEWVAAISGSGDFTHYADSVKGRFTISRDNSKNTVSLQMNNLRAEDTAVYYCAKDEDGGS
LLGYRGQGTLVTVSSFVPVFLPAKPTTTPAPRPPTPAPTIASQPLSLRPEACRPAAGGAVHTRGLD
FACDIYIWAPLAGTCGVLLLSLVITLYCNHRNRSKRSRLLHSDYMNMTPRRPGPTRKHYQPYAPP
RDFAAYRSRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR;
c)
(SEQ ID NO: 73)
MALPVTALLLPLALLLHAARPEVQLLESGGGLVQPGGSLRLSCAASGFTFSSYAMSWVRQAPG
KGLEWVSSISGSGDYIYYADSVKGRFTISRDISKNTLYLQMNSLRAEDTAVYYCAKEGTGANSSL
ADYRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNRSKRSRLLHSDYMNMTPRRPGPTRKHYQPYAPP
RDFAAYRSRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR;
d)
(SEQ ID NO: 74)
MALPVTALLLPLALLLHAARPEVQLLESGGGLIQPGGSLRLSCAASGFTFSSHAMTWVRQAPG
KGLEWVSAISGSGDYTHYADSVKGRFTISRDNSKNTVYLQMNSLRAEDSAVYYCAKDEDGGSL
LGHRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNRSKRSRLLHSDYMNMTPRRPGPTRKHYQPYAPP
RDFAAYRSRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR;
e)
(SEQ ID NO: 75)
MALPVTALLLPLALLLHAARPQVQLVESGGGLVQPGGSLRLSCAASGFTFTNHAMSWVRQAP
GKGLELVSSISGNGRTTYYADSVKGRFTISRDISKNTLDLQMNSLRAEDTAVYYCAKDGGETLV
DSRGQGTLVTVSSFVPVFLPAKPTTTPAPRPPTPAPTIASQPLSLRPEACRPAAGGAVHTRGLDFA
CDIYIWAPLAGTCGVLLLSLVITLYCNHRNKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR;
f)
(SEQ ID NO: 76)
MALPVTALLLPLALLLHAARPQVQLVESGGGLVQPGGSLRLSCAASGFTFSSHAMTWVRQAP
GKGLEWVAAISGSGDFTHYADSVKGRFTISRDNSKNTVSLQMNNLRAEDTAVYYCAKDEDGGS
LLGYRGQGTLVTVSSFVPVFLPAKPTTTPAPRPPTPAPTIASQPLSLRPEACRPAAGGAVHTRGLD
FACDIYIWAPLAGTCGVLLLSLVITLYCNHRNKRGRKKLLYIFKQPFMRPVQTTQEEDGCSCRFP
EEEEGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR;
g)
(SEQ ID NO: 77)
MALPVTALLLPLALLLHAARPEVQLLESGGGLVQPGGSLRLSCAASGFTFSSYAMSWVRQAPG
KGLEWVSSISGSGDYIYYADSVKGRFTISRDISKNTLYLQMNSLRAEDTAVYYCAKEGTGANSSL
ADYRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNKRGRKKLLYIFKQPFMRPVQTTQEEDGCSCRFPE
EEEGGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR;
h)
(SEQ ID NO: 78)
MALPVTALLLPLALLLHAARPEVQLLESGGGLIQPGGSLRLSCAASGFTFSSHAMTWVRQAPG
KGLEWVSAISGSGDYTHYADSVKGRFTISRDNSKNTVYLQMNSLRAEDSAVYYCAKDEDGGSL
LGHRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNKRGRKKLLYIFKQPFMRPVQTTQEEDGCSCRFPE
EEEGGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR;
i)
(SEQ ID NO: 79)
MALPVTALLLPLALLLHAARPQVQLVESGGGLVQPGGSLRLSCAASGFTFTNHAMSWVRQAP
GKGLELVSSISGNGRTTYYADSVKGRFTISRDISKNTLDLQMNSLRAEDTAVYYCAKDGGETLV
DSRGQGTLVTVSSFVPVFLPAKPTTTPAPRPPTPAPTIASQPLSLRPEACRPAAGGAVHTRGLDFA
CDIYIWAPLAGTCGVLLLSLVITLYCNHRNCWLTKKKYSSSVHDPNGEYMFMRAVNTAKKSRL
TDVTLRVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR;
j)
(SEQ ID NO: 80)
MALPVTALLLPLALLLHAARPQVQLVESGGGLVQPGGSLRLSCAASGFTFSSHAMTWVRQAPG
KGLEWVAAISGSGDFTHYADSVKGRFTISRDNSKNTVSLQMNNLRAEDTAVYYCAKDEDGGSL
LGYRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNCWLTKKKYSSSVHDPNGEYMFMRAVNTAKKSR
LTDVTLRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR;
k)
(SEQ ID NO: 81)
MALPVTALLLPLALLLHAARPEVQLLESGGGLVQPGGSLRLSCAASGFTFSSYAMSWVRQAPG
KGLEWVSSISGSGDYIYYADSVKGRFTISRDISKNTLYLQMNSLRAEDTAVYYCAKEGTGANSSL
ADYRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNCWLTKKKYSSSVHDPNGEYMFMRAVNTAKKSR
LTDVTLRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR;
l)
(SEQ ID NO: 82)
MALPVTALLLPLALLLHAARPEVQLLESGGGLIQPGGSLRLSCAASGFTFSSHAMTWVRQAPG
KGLEWVSAISGSGDYTHYADSVKGRFTISRDNSKNTVYLQMNSLRAEDSAVYYCAKDEDGGSL
LGHRGQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNCWLTKKKYSSSVHDPNGEYMFMRAVNTAKKSR
LTDVTLRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR;
m)
(SEQ ID NO: 83)
MALPVTALLLPLALLLHAARPDIVLTQSPPSLAMSLGKRATISCRASESVTILGSHLIYWYQQK
PGQPPTLLIQLASNVQTGVPARFSGSGSRTDFTLTIDPVEEDDVAVYYCLQSRTIPRTFGGGTKLEI
KGSTSGSGKPGSGEGSTKGQIQLVQSGPELKKPGETVKISCKASGYTFRHYSMNWVKQAPGKGL
KWMGRINTESGVPIYADDFKGRFAFSVETSASTAYLVINNLKDEDTASYFCSNDYLYSLDFWGQ
GTALTVSSFVPVFLPAKPTTTPAPRPPTPAPTIASQPLSLRPEACRPAAGGAVHTRGLDFACDIYIW
APLAGTCGVLLLSLVITLYCNHRNRSKRSRLLHSDYMNMTPRRPGPTRKHYQPYAPPRDFAAYR
SRVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR;
n)
(SEQ ID NO: 84)
MALPVTALLLPLALLLHAARPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSFVPVFLPAKPTTTPAPRPPTPAPTIASQPLSLRPEACRPAAGGAVHTRGLDFACDIYIW
APLAGTCGVLLLSLVITLYCNHRNRSKRSRLLHSDYMNMTPRRPGPTRKHYQPYAPPRDFAAYR
SRVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR;
o)
(SEQ ID NO: 85)
MALPVTALLLPLALLLHAARPDIVLTQSPPSLAMSLGKRATISCRASESVTILGSHLIYWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTHYSMNWVKQAPGKGLK
WMGRINTETGEPLYADDFKGRFAFSLETSASTAYLVINNLKNEDTATFFCSNDYLYSCDYWGQG
TTLTVSSFVPVFLPAKPTTTPAPRPPTPAPTIASQPLSLRPEACRPAAGGAVHTRGLDFACDIYIWA
PLAGTCGVLLLSLVITLYCNHRNRSKRSRLLHSDYMNMTPRRPGPTRKHYQPYAPPRDFAAYRS
RVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR;
p)
(SEQ ID NO: 86)
MLLLVTSLLLCELPHPAFLLIPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSAAAFVPVFLPAKPTTTPAPRPPTPAPTIASQPLSLRPEACRPAAGGAVHTRGLDFACDI
YIWAPLAGTCGVLLLSLVITLYCNHRNRSKRSRLLHSDYMNMTPRRPGPTRKHYQPYAPPRDFA
AYRSRVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR;
q)
(SEQ ID NO: 87)
MALPVTALLLPLALLLHAARPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSAAAFVPVFLPAKPTTTPAPRPPTPAPTIASQPLSLRPEACRPAAGGAVHTRGLDFACDI
YIWAPLAGTCGVLLLSLVITLYCNHRNRSKRSRLLHSDYMNMTPRRPGPTRKHYQPYAPPRDFA
AYRSRVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR;
r)
(SEQ ID NO: 88)
MALPVTALLLPLALLLHAARPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSAAAFVPVFLPAKPTTTPAPRPPTPAPTIASQPLSLRPEACRPAAGGAVHTRGLDFACDI
YIWAPLAGTCGVLLLSLVITLYCNHRNRFSVVKRGRKKLLYIFKQPFMRPVQTTQEEDGCSCRFP
EEEEGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR;
s)
(SEQ ID NO: 89)
MALPVTALLLPLALLLHAARPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSAAAFVPVFLPAKPTTTPAPRPPTPAPTIASQPLSLRPEACRPAAGGAVHTRGLDFACDI
YIWAPLAGTCGVLLLSLVITLYCNHRNRRDQRLPPDAHKPPGGGSFRTPIQEEQADAHSTLAKIR
VKFSRSADAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR;
t)
(SEQ ID NO: 90)
MALPVTALLLPLALLLHAARPDIVLTQSPPSLAMSLGKRATISCRASESVTILGSHLIHWYQQKP
GQPPTLLIQLASNVQTGVPARFSGSGSRTDFTLTIDPVEEDDVAVYYCLQSRTIPRTFGGGTKLEIK
GSTSGSGKPGSGEGSTKGQIQLVQSGPELKKPGETVKISCKASGYTFTDYSINWVKRAPGKGLK
WMGWINTETREPAYAYDFRGRFAFSLETSASTAYLQINNLKYEDTATYFCALDYSYAMDYWGQ
GTSVTVSSAAAFVPVFLPAKPTTTPAPRPPTPAPTIASQPLSLRPEACRPAAGGAVHTRGLDFACDI
YIWAPLAGTCGVLLLSLVITLYCNHRNRFSVVKRGRKKLLYIFKQPFMRPVQTTQEEDGCSCRFP
EEEEGGCELRRDQRLPPDAHKPPGGGSFRTPIQEEQADAHSTLAKIRVKFSRSADAPAYQQGQNQ
LYNELNLGRREEYDVLDKRRGRDPEMGGKPRRKNPQEGLYNELQKDKMAEAYSEIGMKGERR
RGKGHDGLYQGLSTATKDTYDALHMQALPPR;
u)
(SEQ ID NO: 91)
MALPVTALLLPLALLLHAARPQVKLEESGGGLVQAGRSLRLSCAASEHTFSSHVMGWFRQAP
GKERESVAVIGWRDISTSYADSVKGRFTISRDNAKKTLYLQMNSLKPEDTAVYYCAARRIDAAD
FDSWGQGTQVTVSSGGGGSEVQLVESGGGLVQAGGSLRLSCAASGRTFTMGWFRQAPGKEREF
VAAISLSPTLAYYAESVKGRFTISRDNAKNTVVLQMNSLKPEDTALYYCAADRKSVMSIRPDYW
GQGTQVTVSSTSTTTPAPRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR;
and
v)
(SEQ ID NO: 92)
MAFLWLLSCWALLGTTFGDYKDDDDKGGGGSGGGGSMGSWSEFWVRLGAIRERLDALGGS
EAELAAFEKEIAAFESELQAYKGKGNPEVEKLRYTAATIRRFLQAYRHNGGGGSGGGGSGTTTP
APRPPTPAPTIASQPLSLRPEACRPAAGGAVHTRGLDFACDIYIWAPLAGTCGVLLLSLVITLYCK
RGRKKLLYIFKQPFMRPVQTTQEEDGCSCRFPEEEEGGCELRVKFSRSADAPAYKQGQNQLYNE
LNLGRREEYDVLDKRRGRDPEMGGKPRRKNPQEGLYNELQKDKMAEAYSEIGMKGERRRGKG
HDGLYQGLSTATKDTYDALHMQALPPR.

Embodiment 511. The multi-component cellular therapy product of any one of embodiments 493-510, wherein the product comprises a dose of CAR+ cells that ranges from approximately 1.0×105 to approximately 5.0×106 CAR+ cells per kilogram of body weight of a human subject receiving the product, or from approximately 4.0×105 to 1.0×109 CAR+ cells.

Embodiment 512. The multi-component cellular therapy product of any one of embodiments 284-511, wherein the one or more cells comprising a chimeric receptor are selected from the group consisting of: T cells, Natural Killer (NK) cells, cytotoxic T lymphocytes (CTLs), regulatory T cells, and Natural Killer T (NKT) cells.

Embodiment 513. The multi-component cellular therapy product of embodiment 512, wherein the one or more cells comprising a chimeric receptor are from an allogeneic donor and have been genetically modified to be to risk of graft versus host disease (GVHD) and/or to prevent allogeneic rejection.

Embodiment 514. A pharmaceutical composition comprising the multi-component cellular therapy product of any one of embodiments 284-513.

Embodiment 515. A method of treating a human subject having or suspected of having a malignancy, comprising administering to the human subject the multi-component cellular therapy product of any one of embodiments 284-513.

Embodiment 516. The method of embodiment 515, wherein the hematologic malignancy is selected from the group consisting of: acute lymphoblastic leukemia (ALL), acute myelogenous leukemia (AML), chronic myelogenous leukemia (CML), multiple myeloma, lymphoma, Hodgkin's lymphoma, non-Hodgkin lymphoma, myelodysplastic syndrome, myelofibrosis, and blastic plasmacytoid dendritic cell neoplasm (BPDCN).

Embodiment 517. The method of embodiment 516, wherein the ALL is B-cell ALL.

Embodiment 518. The method of embodiment 517, wherein the human subject is in complete response with a history of chemotherapy refractory disease.

Embodiment 519. The method of embodiment 518, wherein the chemotherapy refractory disease comprises progression or stable disease after one line of chemotherapy.

Embodiment 520. The method of embodiment 517, wherein the human subject is in complete response with CRLF2 rearrangement, Ph-like phenotype, MLL/KMT2a rearrangement, and/or hypodiploid karyotype.

Embodiment 521. The method of embodiment 520, wherein the Ph-like phenotype comprises Ph+ ALL.

Embodiment 522. The method of embodiment 517, wherein the human subject has persistent or relapsed minimal residual disease (MRD).

Embodiment 523. The method of embodiment 517, wherein the human subject has active B-cell ALL.

Embodiment 524. The method of embodiment 523, wherein the human subject has approximately 5% or greater bone marrow blasts, circulating blasts, and/or extramedullary disease.

Embodiment 525. The method of any one of embodiments 517-524, wherein the B-cell ALL is CD19+.

Embodiment 526. The method of any one of embodiments 514-525, wherein incidence of immune effector cell-associated neurotoxicity syndrome (ICANS) decreases after administration of the multi-component cellular therapy, relative to a corresponding human subject that has been administered only a CAR-T monotherapy.

Embodiment 527. The method of any one of embodiments 514-526, wherein the multi-component cellular therapy prevents development of immune effector cell-associated neurotoxicity syndrome (ICANS) for a period that ranges from approximately 1 month to approximately 6 months, or longer.

Embodiment 528. The method of any one of embodiments 514-527, wherein incidence of cytokine release syndrome (CRS) decreases after administration of the multi-component cellular therapy, relative to a corresponding human subject that has been administered only a CAR-T monotherapy.

Embodiment 529. The method of any one of embodiments 514-528, wherein severity of cytokine release syndrome (CRS) decreases after administration of the multi-component cellular therapy, relative to a corresponding human subject that has been administered only a CAR-T monotherapy.

Embodiment 530. The method of any one of embodiments 514-529, wherein incidence of graft vs host disease (GVHD) decreases after administration of the multi-component cellular therapy, relative to a corresponding human subject that has been administered only an allogeneic CAR-T monotherapy, or relative to a corresponding human subject that has been administered a standard myeloablative allogeneic hematopoietic stem cell transplant (alloHSCT) without a CAR-T therapy.

Embodiment 531. The method of any one of embodiments 514-530, wherein the multi-component cellular therapy prevents development of graft vs host disease (GVHD) for a period that ranges from approximately 1 month to approximately 6 months, or longer.

Embodiment 532. The method of any one of embodiments 514-531, wherein incidence of complete response increases after administration of the multi-component cellular therapy, relative to a corresponding human subject that has been administered a CAR-T therapy without administering any population of isolated CD45+ cells.

Embodiment 533. The method of any one of embodiment 514-532, wherein incidence of minimal residual disease (MRD) decreases after administration of the multi-component cellular therapy, relative to a corresponding human subject that has been administered only a CAR-T monotherapy.

Embodiment 534. The method of any one of embodiments 514-533, wherein the multi-component cellular therapy prevents development of minimal residual disease (MRD) for a period that ranges from approximately 1 month to approximately 6 months, or longer.

Embodiment 535. The method of any one of embodiments 514-534, wherein incidence of Grade III-IV acute GVHD (aGVHD) 42 or more days after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 536. The method of any one of embodiments 514-535, wherein incidence of relapse decreases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 537. The method of any one of embodiments 514-536, wherein incidence of disease progression decreases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 538. The method of any one of embodiments 514-537, wherein incidence of non-relapse mortality (NRM) decreases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 539. The method of any one of embodiments 514-538, wherein incidence of secondary graft failure decreases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 540. The method of any one of embodiments 514-539, wherein incidence of progression-free survival (PRS) increases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 541. The method of any one of embodiments 514-540, wherein incidence of overall survival increases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 542. The method of any one of embodiments 514-541, wherein incidence of Grade 3 or higher infectious disease complications decreases after administration of the multi-component cellular therapy decreases, relative to a corresponding human subject that has been administered only a CAR-T monotherapy, or relative to a corresponding human subject that has been administered a standard myeloablative allogeneic hematopoietic stem cell transplant (alloHSCT) without a CAR-T therapy.

Embodiment 543. The method of any one of embodiments 514-542, wherein the human subject is approximately 3 months of age or older, approximately 6 months of age or older, approximately 9 months of age or older, approximately 12 months of age or older, approximately 18 months of age or older, approximately 2 years of age or older, approximately 3 years of age or older, approximately 4 years of age or older, approximately 5 years of age or older, approximately 6 years of age or older, approximately 7 years of age or older, approximately 8 years of age or older, approximately 9 years of age or older, approximately 10 years of age or older, approximately 11 years of age or older, approximately 12 years of age or older, approximately 13 years of age or older, approximately 14 years of age or older, approximately 15 years of age or older, approximately 16 years of age or older, approximately 17 years of age or older, approximately 18 years of age or older, approximately 19 years of age or older, approximately 20 years of age or older, or approximately 21 years of age or older.

Embodiment 544. The method of any one of embodiments 514-543, wherein the human subject is between approximately 18 years of age to approximately 65 years of age.

Embodiment 545. The method of any one of embodiments 514-543, wherein the human subject is between approximately 18 years of age to approximately 75 years of age.

Embodiment 546. A pharmaceutical composition comprising a multi-component cellular therapy product, wherein the multi-component cellular therapy product comprises:
(a) one or more allogeneic cells comprising a chimeric receptor; and
(b) an immune-modulating cell therapy product,
wherein the one or more allogeneic cells and/or one or more cells of the immune-modulating cell therapy product are from an HLA-compatible donor, relative to a human subject receiving the multi-component cellular therapy product.

Embodiment 547. An engineered cellular graft comprising:
(a) one or more allogeneic cells comprising a chimeric receptor; and
(b) an immune-modulating cell therapy product,
wherein the one or more allogeneic cells and/or one or more cells of the immune-modulating cell therapy product are from an HLA-compatible donor, relative to a human subject receiving the multi-component cellular therapy product.

Embodiment 548. A cellular therapy kit comprising the multi-component cellular therapy product of any one of embodiments 284-513.

Embodiment 549. The cellular therapy kit of embodiment 548, wherein the kit further comprises written instructions for using the cellular therapy for treating a hematologic malignancy in a human subject.

Embodiment 550. A kit for use in preparation of a cellular therapy product, the kit comprising:
(a) an anti-human CD34 affinity reagent;
(b) an anti-human CD25 affinity reagent;
(c) a vector comprising a nucleic acid encoding a chimeric receptor; and
(d) a transfection reagent.

Embodiment 551. The kit of embodiment 550, wherein the kit further comprises written instructions for using the anti-human CD34 affinity reagent to isolate HSPCs from a donor blood sample or a donor bone marrow sample.

Embodiment 552. The kit of embodiment 551, wherein the written instructions further comprise instructions for using the anti-human CD25 affinity reagent to isolate Tregs from the donor blood sample or the donor bone marrow sample.

Embodiment 553. The kit of embodiment 551 or embodiment 552, wherein the written instructions further comprise instructions for using the transfection reagent to introduce the vector into one or more cells from the donor blood sample or the donor bone marrow sample.

Embodiment 554. A method of preparing a multi-component cellular therapy product comprising:
a) obtaining one or more peripheral blood samples or bone marrow samples from a donor that is allogeneic with respect to a human subject receiving the product;
b) collecting a first population of cells from the one or more peripheral blood samples or bone marrow samples and transfecting the population of cells with a vector comprising a nucleic acid encoding the chimeric receptor of any one of embodiments 284-513;
c) combining the transfected population of cells with one or more cryoprotectants and cryopreserving the transfected population of cells;
d) collecting a second population of cells from the one or more peripheral blood samples or bone marrow samples and combining the second population of cells with one or more cryoprotectants and cryopreserving the second population of cells,
wherein the second population of cells comprises conventional CD3+ T cells (Tcons);
e) sorting a third population of cells from the one or more peripheral blood samples or bone marrow samples and with one or more immune-separation particles (ISPs) specific for CD34 to obtain a CD34-enriched cell population and a CD34-depleted cell population,
wherein the CD34-enriched cell population comprises CD34+ hematopoietic stem and progenitor cells (HSPCs);
f) sorting the CD34-depleted cell population with one or more ISPs specific for CD25 to obtain a CD25-enriched cell population and a CD25-depleted cell population; and
g) sorting the CD25-enriched cell population with one or more ISPs specific for CD4 and one or more ISPs specific for CD127, and selecting a CD4+ and CD127dim cell population,
wherein the CD4+ and CD127dim cell population comprises CD4+CD25+CD127dim regulatory T cells (Tregs),
wherein the multi-component cellular therapy product comprises the transfected cell population, the Tcons, the HSPCs, and the Tregs.

Embodiment 555. The method of embodiment 554, wherein the first population of cells comprises the Tcons.

Embodiment 556. The method of embodiment 554, wherein the first population of cells comprises the HSPCs.

Embodiment 557. The method of embodiment 554, wherein the CD4+ and CD127dim cell population is the population of cells transfected with the vector.

Embodiment 558. The method of any of the preceding embodiments, wherein the chimeric receptor comprises the CAR of tisagenlecleucel, axicabtagene ciloleucel, brexucabtagene autoleucel, lisocabtagene maraleucel, idecabtagene vicleucel, or ciltacabtagene autoleucel.

Embodiment 559. The multi-component cellular therapy product of any of the preceding embodiments, wherein the chimeric receptor comprises the CAR of tisagenlecleucel, axicabtagene ciloleucel, brexucabtagene autoleucel, lisocabtagene maraleucel, idecabtagene vicleucel, or ciltacabtagene autoleucel.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Clinical Study

### Background

Rationale to Administer the Engineered Graft Followed by Donor 19-22-BBz CAR T Cells for Patient with ALL

Retrospective analysis of adults with B-cell ALL treated in prior studies found that 102 patients received their first allo-HCT with myeloablative conditioning from 2014 to 2021. For the entire cohort, outcomes at 4 years found an event-free survival of 54.9% (95% confidence interval (CI), 44.5-67.6), GVHD- and relapse-free survival of 22.3% (95% CI, 14.5-34.3), and overall survival of 66.2% (95% CI, 56-78.3).

These data suggest that novel approaches in combination with myeloablative conditioning followed by allo-HCT may be needed to improve patient outcomes. Given that need, a trial was designed where patients received a myeloablative conditioning regimen and the Tcon+Treg+CD34+ HSPC engineered graft followed by the addition of donor CD19/CD22-CAR T cells as an approach to improve outcomes in patients with high-risk B-cell ALL. TABLE 10 contains exemplary sequences of CD19/CD22 CAR constructs for use in this trial.

TABLE 10

| SEQ | | |
|---|---|---|
| ID NO | Name | Sequence |
| 1 | CD22_HC-CDR 1 | GDSVSSNSAA |
| 2 | CD22_HC-CDR 2 | TYYRSKWYN |
| 3 | CD22_HC-CDR 3 | AREVTGDLEDAFDI |
| 4 | CD22_LC-CDR 1 | QTIWSY |
| 5 | CD22_LC-CDR 2 | AAS |
| 6 | CD22_LC-CDR 3 | QQSYSIPQT |
| 7 | CD19_HC-CDR1 | GVSLPDYG |
| 8 | CD19_HC-CDR2 | IWGSETT |
| 9 | CD19_HC-CDR3 | AKHYYYGGSYAMDY |
| 10 | CD19_LC-CDR1 | QDISKY |
| 11 | CD19_LC-CDR2 | HTS |
| 12 | CD19_LC-CDR3 | QQGNTLPYT |
| 13 | CD22 HCVR | QVQLQQSGPGLVKPSQTLSLTCAISGDSVSSNSAAWN |
| | | WIRQSPSRGLEWLGRTYYRSKWYNDYAVSVKSRITIN |
| | | PDTSKNQFSLQLNSVTPEDTAVYYCAREVTGDLEDAF |
| | | DIWGQGTMVTVSS |
| 14 | CD22 LCVR | DIQMTQSPSSLSASVGDRVTITCRASQTIWSYLNWYQQ |
| | | RPGKAPNLLIYAASSLQSGVPSRFSGRGSGTDFTLTISS |
| | | LQAEDFATYYCQQSYSIPQTFGQGTKLEIK |
| 15 | CD19 HCVR | EVKLQESGPGLVAPSQSLSVTCTVSGVSLPDYGVSWIR |
| | | QPPRKGLEWLGVIWGSETTYYNSALKSRLTIIKDNSKS |
| | | QVFLKMNSLQTDDTAIYYCAKHYYYGGSYAMDYWG |
| | | QGTSVTVSS |
| 16 | CD19 LCVR | DIQMTQTTSSLSASLGDRVTISCRASQDISKYLNWYQQ |
| | | KPDGTVKLLIYHTSRLHSGVPSRFSGSGSGTDYSLTISN |
| | | LEQEDIATYFCQQGNTLPYTFGGGTKLEIT |
| 17 | HCVR/LCVR 218 Linker | GSTSGSGKPGSGEGSTKG |
| | Sequence | |
| 18 | CD19 scFv/CD22scFv | GGGGS GGGGS GGGGS GGGGS GGGGS |
| | Linker | |
| 19 | Short Linker Sequence | GGGGS |
| 20 | CD22 scFv with 218 | QVQLQQSGPGLVKPSQTLSLTCAISGDSVSSNSAAWN |
| | Linker | WIRQSPSRGLEWLGRTYYRSKWYNDYAVSVKSRITIN |
| | | PDTSKNQFSLQLNSVTPEDTAVYYCAREVTGDLEDAF |
| | | DIWGQGTMVTVSSGSTSGSGKPGSGEGSTKGDIQMTQ |
| | | SPSSLSASVGDRVTITCRASQTIWSYLNWYQQRPGKAP |
| | | NLLIYAASSLQSGVPSRFSGRGSGTDFTLTISSLQAEDF |
| | | ATYYCQQSYSIPQTFGQGTKLEIK |
| 21 | CD22 scFV with Short | QVQLQQSGPGLVKPSQTLSLTCAISGDSVSSNSAAWN |
| | Linker | WIRQSPSRGLEWLGRTYYRSKWYNDYAVSVKSRITIN |
| | | PDTSKNQFSLQLNSVTPEDTAVYYCAREVTGDLEDAF |
| | | DIWGQGTMVTVSSGGGGSDIQMTQSPSSLSASVGDRV |
| | | TITCRASQTIWSYLNWYQQRPGKAPNLLIYAASSLQSG |
| | | VPSRFSGRGSGTDFTLTISSLQAEDFATYYCQQSYSIPQ |
| | | TFGQGTKLEIK |
| 22 | CD19 scFV with 218 | DIQMTQTTSSLSASLGDRVTISCRASQDISKYLNWYQQ |
| | Linker | KPDGTVKLLIYHTSRLHSGVPSRFSGSGSGTDYSLTISN |
| | | LEQEDIATYFCQQGNTLPYTFGGGTKLEITGSTSGSGK |
| | | PGSGEGSTKGEVKLQESGPGLVAPSQSLSVTCTVSGVS |
| | | LPDYGVSWIRQPPRKGLEWLGVIWGSETTYYNSALKS |
| | | RLTIIKDNSKSQVFLKMNSLQTDDTAIYYCAKHYYYG |
| | | GSYAMDYWGQGTSVTVSS |
| 23 | CD19 LCVR + short | DIQMTQTTSSLSASLGDRVTISCRASQDISKYLNWYQQ |
| | linker + CD22 HCVR + | KPDGTVKLLIYHTSRLHSGVPSRFSGSGSGTDYSLTISN |
| | 218 linker + CD22 LCVR + | LEQEDIATYFCQQGNTLPYTFGGGTKLEITGGGGSQVQ |
| | short linker + | LQQSGPGLVKPSQTLSLTCAISGDSVSSNSAAWNWIRQ |
| | CD19 HCVR + S | SPSRGLEWLGRTYYRSKWYNDYAVSVKSRITINPDTS |
| | | KNQFSLQLNSVTPEDTAVYYCAREVTGDLEDAFDIWG |
| | | QGTMVTVSSGSTSGSGKPGSGEGSTKGDIQMTQSPSSL |
| | | SASVGDRVTITCRASQTIWSYLNWYQQRPGKAPNLLI |
| | | YAASSLQSGVPSRFSGRGSGTDFTLTISSLQAEDFATY |
| | | YCQQSYSIPQTFGQGTKLEIKGGGGSEVKLQESGPGLV |
| | | APSQSLSVTCTVSGVSLPDYGVSWIRQPPRKGLEWLG |
| | | VIWGSETTYYNSALKSRLTIIKDNSKSQVFLKMNSLQT |
| | | DDTAIYYCAKHYYYGGSYAMDYWGQGTSVTVSSS |
| 24 | CD22 HCVR + CD22 | QVQLQQSGPGLVKPSQTLSLTCAISGDSVSSNSAAWN |
| | LCVR + (GGGGS)5 [SEQ | WIRQSPSRGLEWLGRTYYRSKWYNDYAVSVKSRITIN |
| | ID NO: 106] linker + CD19 | PDTSKNQFSLQLNSVTPEDTAVYYCAREVTGDLEDAF |
| | LCVR + 218 linker + CD19 | DIWGQGTMVTVSSGGGGSDIQMTQSPSSLSASVGDRV |
| | HCVR | TITCRASQTIWSYLNWYQQRPGKAPNLLIYAASSLQSG |
| | | VPSRFSGRGSGTDFTLTISSLQAEDFATYYCQQSYSIPQ |
| | | TFGQGTKLEIKGGGGSGGGGSGGGGSGGGGSGGGGS |
| | | DIQMTQTTSSLSASLGDRVTISCRASQDISKYLNWYQQ |
| | | KPDGTVKLLIYHTSRLHSGVPSRFSGSGSGTDYSLTISN |
| | | LEQEDIATYFCQQGNTLPYTFGGGTKLEITGSTSGSGK |
| | | PGSGEGSTKGEVKLQESGPGLVAPSQSLSVTCTVSGVS |
| | | LPDYGVSWIRQPPRKGLEWLGVIWGSETTYYNSALKS |
| | | RLTIIKDNSKSQVFLKMNSLQTDDTAIYYCAKHYYYG |
| | | GSYAMDYWGQGTSVTVSS |
| 25 | Leader sequence | MLLLVTSLLLCELPHPAFLLIP |
| 26 | CD8 transmembrane domain | IYIWAPLAGTCGVLLLSLVITLYC |
| 27 | 4-1BB signaling domain | KRGRKKLLYIFKQPFMRPVQTTQEEDGCSCRFPEEEEG |
| | | GCEL |
| 28 | CD3-zeta signaling domain | RVKFSRSADAPAYXQGQNQLYNELNLGRREEYDVLD |
| | X = Gln (Q) or Lys (K) | KRRGRDPEMGGKPRRKNPQEGLYNELQKDKMAEAYS |
| | | EIGMKGERRRGKGHDGLYQGLSTATKDTYDALHMQA |
| | | LPPR |
| 29 | Full CAR sequence- | DIQMTQTTSSLSASLGDRVTISCRASQDISKYLNWYQQ |
| | CD19LCVR/CD22HCVR/ | KPDGTVKLLIYHTSRLHSGVPSRFSGSGSGTDYSLTISN |
| | CD22 LCVR/CD19 HCVR | LEQEDIATYFCQQGNTLPYTFGGGTKLEITGGGGSQVQ |
| | and CD3-zeta K | LQQSGPGLVKPSQTLSLTCAISGDSVSSNSAAWNWIRQ |
| | | SPSRGLEWLGRTYYRSKWYNDYAVSVKSRITINPDTS |
| | | KNQFSLQLNSVTPEDTAVYYCAREVTGDLEDAFDIWG |
| | | QGTMVTVSSGSTSGSGKPGSGEGSTKGDIQMTQSPSSL |
| | | SASVGDRVTITCRASQTIWSYLNWYQQRPGKAPNLLI |
| | | YAASSLQSGVPSRFSGRGSGTDFTLTISSLQAEDFATY |
| | | YCQQSYSIPQTFGQGTKLEIKGGGGSEVKLQESGPGLV |
| | | APSQSLSVTCTVSGVSLPDYGVSWIRQPPRKGLEWLG |
| | | VIWGSETTYYNSALKSRLTIIKDNSKSQVFLKMNSLQT |
| | | DDTAIYYCAKHYYYGGSYAMDYWGQGTSVTVSSSGT |
| | | TTPAPRPPTPAPTIASQPLSLRPEACRPAAGGAVHTRGL |
| | | DFACDIYIWAPLAGTCGVLLLSLVITLYCKRGRKKLLY |
| | | IFKQPFMRPVQTTQEEDGCSCRFPEEEEGGCELRVKFS |
| | | RSADAPAYKQGQNQLYNELNLGRREEYDVLDKRRGR |
| | | DPEMGGKPRRKNPQEGLYNELQKDKMAEAYSEIGMK |
| | | GERRRGKGHDGLYQGLSTATKDTYDALHMQALPPR |
| 30 | Full CAR sequence- | QVQLQQSGPGLVKPSQTLSLTCAISGDSVSSNSAAWN |
| | CD22HCVR/CD22LCVR/ | WIRQSPSRGLEWLGRTYYRSKWYNDYAVSVKSRITIN |
| | CD19LCVR/CD19HCVR | PDTSKNQFSLQLNSVTPEDTAVYYCAREVTGDLEDAF |
| | and CD3-zeta K | DIWGQGTMVTVSSGGGGSDIQMTQSPSSLSASVGDRV |
| | | TITCRASQTIWSYLNWYQQRPGKAPNLLIYAASSLQSG |
| | | VPSRFSGRGSGTDFTLTISSLQAEDFATYYCQQSYSIPQ |
| | | TFGQGTKLEIKGGGGSGGGGSGGGGSGGGGSGGGGS |
| | | DIQMTQTTSSLSASLGDRVTISCRASQDISKYLNWYQQ |
| | | KPDGTVKLLIYHTSRLHSGVPSRFSGSGSGTDYSLTISN |
| | | LEQEDIATYFCQQGNTLPYTFGGGTKLEITGSTSGSGK |
| | | PGSGEGSTKGEVKLQESGPGLVAPSQSLSVTCTVSGVS |
| | | LPDYGVSWIRQPPRKGLEWLGVIWGSETTYYNSALKS |
| | | RLTIIKDNSKSQVFLKMNSLQTDDTAIYYCAKHYYYG |
| | | GSYAMDYWGQGTSVTVSSTTTPAPRPPTPAPTIASQPL |
| | | SLRPEACRPAAGGAVHTRGLDFACDIYIWAPLAGTCG |
| | | VLLLSLVITLYCKRGRKKLLYIFKQPFMRPVQTTQEED |
| | | GCSCRFPEEEEGGCELRVKFSRSADAPAYKQGQNQLY |
| | | NELNLGRREEYDVLDKRRGRDPEMGGKPRRKNPQEG |
| | | LYNELQKDKMAEAYSEIGMKGERRRGKGHDGLYQGL |
| | | STATKDTYDALHMQALPPR |
| 33 | CD8 hinge domain | TTTPAPRPPTPAPTIASQPLSLRPEACRPAAGGAVHTRG |
| | | LDFACD |
| 39 | CD19 LCVR + short | DIQMTQTTSSLSASLGDRVTISCRASQDISKYLNWYQQ |
| | linker + CD22 HCVR + | KPDGTVKLLIYHTSRLHSGVPSRFSGSGSGTDYSLTISN |
| | 218 linker + CD22 LCVR + | LEQEDIATYFCQQGNTLPYTFGGGTKLEITGGGGSQVQ |
| | CD19 HCVR + SG + | LQQSGPGLVKPSQTLSLTCAISGDSVSSNSAAWNWIRQ |
| | CD8 hinge + CD8 TM + | SPSRGLEWLGRTYYRSKWYNDYAVSVKSRITINPDTS |
| | 4-1BB +CD3-zeta with Q | KNQFSLQLNSVTPEDTAVYYCAREVTGDLEDAFDIWG |
| | | QGTMVTVSSGSTSGSGKPGSGEGSTKGDIQMTQSPSSL |
| | | SASVGDRVTITCRASQTIWSYLNWYQQRPGKAPNLLI |
| | | YAASSLQSGVPSRFSGRGSGTDFTLTISSLQAEDFATY |
| | | YCQQSYSIPQTFGQGTKLEIKGGGGSEVKLQESGPGLV |
| | | APSQSLSVTCTVSGVSLPDYGVSWIRQPPRKGLEWLG |
| | | VIWGSETTYYNSALKSRLTIIKDNSKSQVFLKMNSLQT |
| | | DDTAIYYCAKHYYYGGSYAMDYWGQGTSVTVSSSGT |
| | | TTPAPRPPTPAPTIASQPLSLRPEACRPAAGGAVHTRGL |
| | | DFACDIYIWAPLAGTCGVLLLSLVITLYCKRGRKKLLY |
| | | IFKQPFMRPVQTTQEEDGCSCRFPEEEEGGCELRVKFS |
| | | RSADAPAYQQGQNQLYNELNLGRREEYDVLDKRRGR |
| | | DPEMGGKPRRKNPQEGLYNELQKDKMAEAYSEIGMK |
| | | GERRRGKGHDGLYQGLSTATKDTYDALHMQALPPR |
| 40 | Full CAR sequence- | QVQLQQSGPGLVKPSQTLSLTCAISGDSVSSNSAAWN |
| | CD22HCVR/CD22LCVR/ | WIRQSPSRGLEWLGRTYYRSKWYNDYAVSVKSRITIN |
| | CD19LCVR/CD19HCVR | PDTSKNQFSLQLNSVTPEDTAVYYCAREVTGDLEDAF |
| | and CD3-zeta Q | DIWGQGTMVTVSSGGGGSDIQMTQSPSSLSASVGDRV |
| | | TITCRASQTIWSYLNWYQQRPGKAPNLLIYAASSLQSG |
| | | VPSRFSGRGSGTDFTLTISSLQAEDFATYYCQQSYSIPQ |
| | | TFGQGTKLEIKGGGGSGGGGSGGGGSGGGGSGGGGS |
| | | DIQMTQTTSSLSASLGDRVTISCRASQDISKYLNWYQQ |
| | | KPDGTVKLLIYHTSRLHSGVPSRFSGSGSGTDYSLTISN |
| | | LEQEDIATYFCQQGNTLPYTFGGGTKLEITGSTSGSGK |
| | | PGSGEGSTKGEVKLQESGPGLVAPSQSLSVTCTVSGVS |
| | | LPDYGVSWIRQPPRKGLEWLGVIWGSETTYYNSALKS |
| | | RLTIIKDNSKSQVFLKMNSLQTDDTAIYYCAKHYYYG |
| | | GSYAMDYWGQGTSVTVSSTTTPAPRPPTPAPTIASQPL |
| | | SLRPEACRPAAGGAVHTRGLDFACDIYIWAPLAGTCG |
| | | VLLLSLVITLYCKRGRKKLLYIFKQPFMRPVQTTQEED |
| | | GCSCRFPEEEEGGCELRVKFSRSADAPAYQQGQNQLY |
| | | NELNLGRREEYDVLDKRRGRDPEMGGKPRRKNPQEG |
| | | LYNELQKDKMAEAYSEIGMKGERRRGKGHDGLYQGL |
| | | STATKDTYDALHMQALPPR |
| 41 | CD19 scFV with Short | DIQMTQTTSSLSASLGDRVTISCRASQDISKYLNWYQQ |
| | Linker | KPDGTVKLLIYHTSRLHSGVPSRFSGSGSGTDYSLTISN |
| | | LEQEDIATYFCQQGNTLPYTFGGGTKLEITGGGGSEVK |
| | | LQESGPGLVAPSQSLSVTCTVSGVSLPDYGVSWIRQPP |
| | | RKGLEWLGVIWGSETTYYNSALKSRLTIIKDNSKSQVF |
| | | LKMNSLQTDDTAIYYCAKHYYYGGSYAMDYWGQGT |
| | | SVTVSS |

### Study Design

The primary purpose of the study was designed to assess the safety of administering allogenic, donor-derived CD19/CD22-CAR T cells that meet established release specifications in adults with B-cell ALL following a myeloablative conditioning regimen and the alloHSCT (an Tcon+ Treg+ CD34+ HSPC engineered graft) to determine if the alloHSCT treatment augments graft versus leukemia without increasing acute GVHD or graft failure.

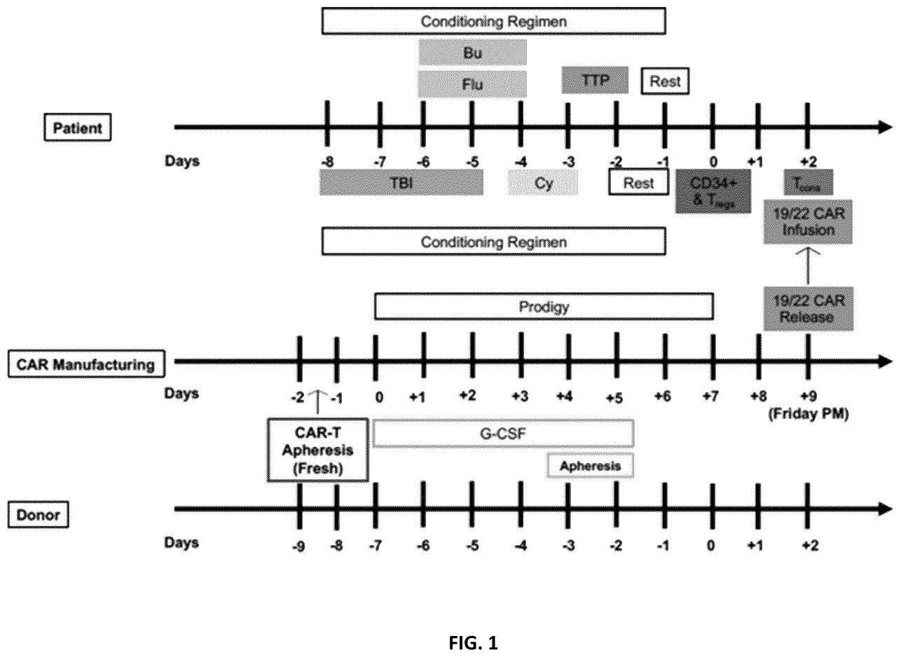

Initial study patients received a myeloablative preparative regimen consisting of chemotherapy+/−total body irradiation. On Day 0, patients received a Treg CD34+ HSPC infusion. On Day +2, patients received an infusion of conventional T cells (Tcon) followed by infusion of donor CD19/CD22-CAR T cells (FIG. 1). The addition of donor CD19/CD22-CAR T cells as a strategy to decrease relapse in patients with B-cell ALL is assessed.

The study includes three cohorts of 3 to 9 patients per cohort. A Bayesian dose escalation design is used for the dosing of the donor CD19/CD22-CAR T cells. There are three dose levels: 1×10⁶, 2×10⁶ and 3×10⁶ donor CD19/CD22 CAR positive T cells per kg (±20%). The starting point is Dose Level 1. If toxicity is noted at Dose Level 1, then the Dose Level −1 is as follows: 0.5×10⁶ CAR+ cells/kg. The Bayesian dose escalation design was generated by allowing for the three dose levels (1, 2, 3) and the step-down level (−1).

The scientific justification for this study design is noted below:
Preclinical and clinical findings demonstrate that donor CAR T cells can be administered following allo-HCT without inducing increased GVHD.
Retrospective analysis of patients with B-cell ALL treated at our center found that 102 patients received their first allo-HCT with myeloablative conditioning from 2014 to 2021. For the entire cohort, outcomes at 4 years found an event-free survival of 54.9% (95% confidence interval (CI), 44.5-67.6), GVHD- and relapse-free survival of 22.3% (95% CI, 14.5-34.3), and overall survival of 66.2% (95% CI, 56-78.3).
These data suggest that novel approaches in combination with myeloablative conditioning followed by allo-HCT may be needed to improve patient outcomes.

This is an open-label Phase I dose escalation study to assess the safety of donor CD19/CD22CAR T cells plus an infusion of an engineered graft of Treg plus CD34+ HSPC plus Tcon (alloHSCT). Patients with B-cell ALL at high risk for relapse or progression who have a matched-related donor are candidates for enrollment on the study.

### Dosing

The doses in TABLE 11 below are used for the donor derived CD19/CD22CAR T cells (±20%). A Bayesian dose escalation design is used.

TABLE 11

| Dose Level | CD3+ Tcon Dose | CAR-T cell Dose |
|---|---|---|
| −1 | 3 × 10⁶ cells/kg | 0.5 × 10⁶ CAR+ cells/kg |
| 1 | 3 × 10⁶ cells/kg | 1 × 10⁶ CAR+ cells/kg |
| 2 | 3 × 10⁶ cells/kg | 2 × 10⁶ CAR+ cells/kg |
| 3 | 3 × 10⁶ cells/kg | 3 × 10⁶ CAR+ cells/kg |

Dose and timing of the administration of DLI are both important factors to consider in regard to their GVHD potential. To minimize the potential for GVHD, the study design administers a single dose of donor CD19/CD22-CAR T cells at 10⁶ cell/kg in the setting of Tregs and tacrolimus for GVHD prophylaxis.

Based on data from a previous autologous CD19/CD22 study, the recommended Phase II dose (RP2D) for CD19/22-BBz-CAR T cells in adult B-ALL was defined as 3×10⁶ CAR T cells/kg and selected as the target for the Dose 3 Cohort. Dose 1 and Dose 2 Cohorts receive lower doses of donor CD19/CD22-CAR T cells so that the risk GVHD and graft failure can be monitored.

### Protocol Synopsis

Primary Objective: Assess the feasibility of manufacturing and the safety of administering allogeneic, donor CD19/CD22-CAR T cells that meet established release specifications and determine the MTD in adults with B-cell acute lymphoblastic leukemia (ALL) following a myeloablative conditioning regimen and the engineered graft of Treg plus CD34+ HSPC plus Tcons.

### Hypothesis

For patients with ALL receiving a myeloablative conditioning regimen and the engineered graft from a matched-related donor, the infusion of donor conventional CD19/CD22-CAR T cells will augment graft versus leukemia without increasing acute graft versus host disease (GVHD) or graft failure.

### Primary Endpoints:

1. Ability to manufacture donor CD19/CD22-CAR T cells that meet established release specifications as assessed by the number of product successes—with a goal of 2 of 3 products, 3 of 6 products, 4 of 12 products, or 5 of 18 products.
2. Safety of the donor CD19/CD22-CAR T cells plus the engineered graft as assessed by the incidence of engraftment without Grade III to IV acute GVHD at Day 42 to determine the MTD.

Secondary Objectives: Determine the cumulative incidence of relapse or progression of disease as well as non-relapse mortality (NRM) following donor CD19/CD22-CAR T cells plus engineered graft infusion; assess the rate of secondary graft failure following donor CD19/CD22-CAR T cells plus engineered graft infusion; analyze progression-free (PFS) and overall (OS) survival following a donor CD19/CD22-CAR T cell infusion plus engineered graft infusion; and evaluate the incidence of greater than Grade 3 infectious disease complications.

### Secondary Endpoints:

1. Relapse, disease progression and NRM at 1 year.
2. Frequency of secondary graft failure within 1 year.
3. PFS and OS at 1 year following engineered graft infusion plus donor CD19/CD22-CAR T cell infusion.
4. Greater than Grade 3 infectious disease complication within 1 year.

Sample Size: The study consists of 12-18 patients in three cohorts consisting of 3 to 9 patients per cohort.

### Patient Inclusion Criteria

Disease Status of B-cell ALL: Subjects in CR must have a history of chemotherapy refractory disease defined as progression or stable disease after one line of chemotherapy, or relapsed disease after achieving prior CR OR must have other high risk ALL features including: CRLF2 rearrangement, Ph-like phenotype, MLL/KMT2a rearrangement, or hypodiploid karyotype. Subjects with persistent or relapsed minimal residual disease (MRD) (by flow cytometry, PCR, FISH, or next generation sequencing) require verification of MRD in the peripheral blood or bone marrow on two occasions at least 2 weeks apart. Subjects with active ALL (defined as >=5% bone marrow blasts, circulating blasts, or extramedullary disease) are eligible. Age ≥18 and ≤65 years (i.e., from age 18 to <66 years old) at the time of enrollment. Eastern cooperative oncology group (ECOG) performance status of 0, 1, or 2; or Karnofsky ≥60%.

CD19 expression is required any time since diagnosis. CD19 expression is detected by immunohistochemistry or by flow cytometry. The choice of whether to use flow cytometry or immunohistochemistry is determined by what is the most easily available tissue sample in each subject. In general, immunohistochemistry is used for lymph node biopsies, flow cytometry is used for peripheral blood and bone marrow samples. Patients receiving prior CD19 CAR T cell or blinatumomab are eligible if there is no documented history of CD19 negativity on the malignant cells.

Subjects must have an HLA matched related donor willing to undergo unstimulated apheresis for T cell collection for CAR T cell generation followed by GCSF mobilized apheresis for HSC/Treg graft. Matched related donor who is defined as an 8/8 match for HLA-A, -B, -C, and -DRB1, all typed using DNA-based high-resolution methods.

### Treatment Summary

Following enrollment, the patient's allograft donor has a leukapheresis product obtained from peripheral blood before stem cell mobilization to generate the CAR T cells (FIG. 1). T cells are enriched from the leukapheresis product, activated and genetically modified (using a lentiviral vector) to express a CAR targeting CD19/CD22 (19-22-BBz). These T cells are expanded, and after the appropriate number of cells are generated, the modified T cells can be cryopreserved according to standard operating procedures for later infusion. A second G-CSF mobilized leukapheresis is obtained for the allograft. The second leukapheresis is the source of the following engineered graft infusion product: conventional T-cells (Tcon), regulatory T cells (Treg), and CD34+ hematopoietic progenitor/stem cells (FIG. 1). Patients receive a myeloablative preparative regimen consisting of chemotherapy+/−total body irradiation (FIG. 1). On Transplant Day 0, patients receive the engineered graft infusions of CD34+ HSPC and Treg (FIG. 1). On Day +2, patients receive an infusion of conventional T cells (Tcon) followed by infusion of donor CD19/CD22-CAR T cells (FIG. 1). Graft-versus-host disease prophylaxis initially commences on Day +3 with single agent tacrolimus (recommended target level 6). Serial sampling of blood and bone marrow is performed following treatment to assess toxicity, therapeutic effects, and persistence of the genetically modified T cells.

Initial subjects were administered the treatment protocol described above.

### Study Design

### Overall Design

The intervention model is a Phase 1 open-label, dose-escalation, single group, non-randomized, non-controlled single center trial.

Purpose: Assess the safety of administering allogenic, donor-derived CD19/CD22-CAR T cells that meet established release specifications in adults with B-cell ALL following a myeloablative conditioning regimen and Engineered graft determining if this will augment graft versus leukemia without increasing acute GVHD or graft failure.

Treatment: In this study patients receive a myeloablative preparative regimen consisting of chemotherapy+/−total body irradiation. On Day 0, patients receive an engineered graft (Treg+CD34+ HSPC) infusion. On Day +2, patients receive an infusion of conventional T cells followed by infusion of donor CD19/CD22-CAR T cells. We are investigating the addition of donor CD19/CD22-CAR T cells as a strategy to decrease relapse in patients with B-cell ALL.

Study Cohorts/Arms/Groups: The study consists of three cohorts consisting of 3 to 9 patients per cohort. We employ a Bayesian dose escalation design for the dosing of the donor CD19/CD22-CAR T cells. There are three dose levels: 1×10⁶, 2×10⁶ and 3×10⁶ donor CD19/CD22 CAR positive T cells per kg (±20%). The starting point is Dose Level 1. If toxicity is noted at Dose Level 1, then the Dose Level −1 is as follows: 0.5×10⁶ CAR+ cells/kg. The Bayesian dose escalation design was generated by allowing for the three dose levels (1, 2, 3) and the step-down level (−1).

### Justification for Dose(s)

Targeted therapy with tumor specific T cells has been widely used in cancer therapy. The ability of tumor-specific T cells to eradicate tumor is best illustrated in patients with relapsed chronic myelogenous leukemia (CML) after allo-HCT. In this setting, the adoptive transfer of donor T cells alone is often sufficient to induce a second remission through a graft versus leukemia (GVL) mechanism. Investigators have administered various dose levels of donor leukocyte infusions (DLI), and the DLI has been infused at varying time points post-allo-HCT. In one notable study, investigators treated 38 patients with various hematologic malignancies with a T-cell depleted transplant. Of the 38 patients in the study, 26 patients received DLI of 2×10⁶ cells/kg on Day 30 and 5×10⁷ cells/kg on Day 45 (schedule 1). The overall probability of grade ≥II aGVHD developing was 31.5%, with a 15.5% probability of aGVHD occurring after DLI. The remaining 12 patients received DLI at 107 T cells/kg on Day 30 (schedule 2), and the probability of grade ≥II aGVHD was 100% 21. These data highlight that the dose and timing of the administration of DLI are both important factors to consider in regard to their GVHD potential. To minimize the potential for GVHD, we propose to administer a single dose of donor CD19/CD22-CAR T cells at 10⁶ cell/kg in the setting of Tregs and tacrolimus for GVHD prophylaxis.

Based on data from the autologous CD19/CD22 study, the recommended Phase II dose (RP2D) for CD19/22-BBz-CAR T cells in adult B-ALL was defined as 3×10⁶ CAR T cells/kg. Hence, we selected this dose as our target for the Dose 3 Cohort. Dose 1 and Dose 2 Cohorts receive lower doses of donor CD19/CD22-CAR T cells so that we can monitor for the risk GVHD and graft failure. Investigational Drug: CD19/CD22-CAR T cells

Allogeneic donor-derived T-cells transduced with bivalent lentiviral vector (CD19/CD22-BBz) chimeric antigen receptor (CAR); following preparative chemotherapy regimen.

CD19/C22CAR T cells are administered at a dose of CAR+ cells/kg body weight via IV administration as in TABLE 11.

Donor CD19/CD22CAR T cells are generated from fresh or cryopreserved PBMCs under GMP conditions in a cell manufacturing facility using Miltenyi CliniMACS Prodigy® system. Apheresis products from participating healthy donors are sent to the manufacturing facility through Stanford Bone Marrow Transplant Department's Cellular Therapeutics Facility (CTF); final cell products are be received, and the final formulated product is distributed by the CTF.

Any prepared cells not required for the first infusion or for research or regulatory purposes (including sufficient QC retention vials for testing cryopreserved product stability at time of additional infusions) are cryopreserved by standard techniques. Additional product in excess of that needed for infusion can be utilized for research.

Fresh or cryopreserved PBMCs (depending on the timing of apheresis relative to cell culture, subject condition, and scheduling availability) are used for cell manufacturing. Cells are required to meet the release criteria as outlined in TABLE 12, as well as no evidence for replication competent lentivirus. All procedures take place using good manufacturing process guidelines.

Additional information on the manufacturer facility information, package label, container system, and formulation are provided in the IND Chemistry and Manufacturing Controls (CMC) section.

TABLE 12

| Test | Criteria |
|---|---|
| Cell viability | ≥70% |
| Total Cell Count | within 20% of target dose level |
| CD3+ % | ≥70% |
| CAR+ % | ≥10% |
| Endotoxin | <5 EU/kg |
| Mycoplasma by PCR | Negative |
| RCL | Negative |
| Sterility testing | Negative |

Clinical doses are frozen in cryobags in a controlled rate freezer. Clinical product is then transported to and stored in temperature monitored liquid nitrogen freezer, monitored by an alarm system. Up to 2 clinical doses are be frozen per patient, based on cell expansion. The product expiration is within 6 hours post-thaw.

Cryopreserved cells are thawed at bedside by trained CTF staff and administered over 10 to 20 min+/−5 minutes.

### Investigational Drug: Engineered Graft of Tcon, Treg, and CD34+ HSPC

Purified donor-derived regulatory T-cell (Treg) plus CD34+ hematopoietic progenitor cells. Dose consisting of ≥2×10⁶ HSPC and ≥2×10⁶/kg Treg is based on recipient actual body weight rounded to the nearest kilogram (as assessed during screening). Per institutional standard practice, an appropriate long-term central venous access is placed for administration.

TABLE 13 describes the release tests and specifications for the CD34+ HSPC final formulated drug product.

TABLE 13

| CD34 Post-selection | | | |
|---|---|---|---|
| Treatment Component | | | |
| Release Testing | Assay Type | Method | Specification |
| Purity | Viability | Dye exclusion (7AAD or | ≥70%* |
| | | Trypan Blue) | |
| Purity/Identity | CD34+ cells | Flow Cytometry | ≥50% CD34+ cells* |
| Potency | CD3+ cell | Flow Cytometry | ≤5 × 104 cells/kg |
| | number | | |
| | CD3+ cell | Flow Cytometry | ≤5 × 104 cells/kg |
| | number | | |
| Safety | Sterility | Gram-stain | Negative* |
| Safety | Sterility | Bactec Method | Negative/no growth |
| | | | observed |
| Safety | Endotoxin | Endosafe-PTS Assay or | 5 EU/kg/hour* |
| | | Limulus amoebocyte | |
| | | assay (LAL) | |

*Release specification for engineered graft Drug Product

After cell selection and sampling, the CD34-enriched products are securely stored at 2 to 8° C. in a continuously monitored refrigerator until released for infusion. Product labeling is compliant with ISBT 128 specifications.

TABLE 14 describes the release tests and specifications for the Treg final formulated drug product.

TABLE 14

| Treg Cells Drug | | | |
|---|---|---|---|
| Product Release | | | |
| Testing | Assay Type | Method | Specification |
| Identity | FoxP3+ Cells | Flow Cytometry | ≥80% FoxP3+ of CD45+ |
| | | | events* |
| Potency | Cell count | Automated Cell | To meet cohort dose* |
| | | Count | (2-3 ± 1 × 10⁶ cells//kg) |
| Purity | Viability | Dye exclusion (7AAD | ≥70%* |
| | | or Trypan Blue) | |
| Safety | Sterility | Gram Stain | Negative* |
| Safety | Sterility | 14-day sterility | Negative |
| Safety | Endotoxin | Endosafe-PTS Assay | 5 EU/kg/hour* |
| | | or LAL | |

*Release specification for Treg Drug Product

Treg can be infused at cell dose per PI discretion if the cell dose is out of range.

After processing, the cellular components (drug products), are transferred via validated transport methods to BMT-CTF, which is the “cell pharmacy” for the Stanford Health Care. Product labeling is compliant with ISBT 128 specifications. The BMT-CTF dispenses the cellular components to the respective subjects.

Formulation, Packaging, and Labeling: Components were provided as single dose transfer bags, with an approximate fill volume of 100 mL each for both the Treg and the HSPC components. The Tcon component was provided frozen and stored in the vapor phase of liquid nitrogen, in an approximate volume of 15 mL. Each product consisted of selected cells, Plasma-Lyte A, and human serum albumin, at a pH of approximately 7.4. The bags and/or primary bag container had labels bearing the appropriate label text as required by governing regulatory agencies. Additionally, the Tcon drug product contained 10% DMSO.

Preparation: All cryopreserved products for infusion were thawed either in the cell processing facility or bedside on the day of infusion per CTF SOPs. For Treg, only the volume of suspension containing the prescribed cell dose is released for infusion. Extra cells, if any, could be used for additional assays or discarded according to institutional guidelines. No washing of the thawed cells was performed.

### Treatment Plan

Potential donors are approached for the study after the decision to proceed with transplantation is made and a suitable HLA-matched related donor (8/8) is identified.

Donor study procedures, including informed consent, is overseen at the investigator's site (e.g., related donors who reside near the investigator's site).

Donors need to be available and willing to donate for two separate apheresis collections (for manufacturing of CD19/CD22-CAR T cells and for the engineered graft).

### Donor Premedication

Apheresis procedure, including the machine, collection procedure and supportive care is in accordance with standardized procedures of Stanford Blood and marrow transplantation (BMT)/Apheresis program.

For the mononuclear cell (MNC) collection, typically process 12-25 L of participant's whole blood. For the hematopoietic progenitor cell assay (HPCA), usually process 3.5-4.5 hours.

### G-CSF Mobilization

All donors receive mobilization therapy per institutional guidelines with daily G-CSF. The recommended dose is 10 μg/kg/day SQ (rounded off to the nearest vial size of either 300 or 480 μg). The Mobilization Phase starts on the first day (Day −7±1 day) of administration of G-CSF and continues until Day −2±1 day before the second leukapheresis (Day −3±1 day).

Per institutional standards, plerixafor (e.g., 0.24 mg/kg SC, once) can be administered if recommended by the investigator and/or attending physician to achieve the HSPC dose targets.

While not required per protocol, monitoring donor peripheral blood CD34+ cell counts prior to donation is recommended to identify doors that are mobilizing poorly.

### Cell Collection for Treg CD34+ HSPC

All cellular components are obtained from matched donors mobilized with G-CSF [filgrastim (Neupogen) or equivalent] and collected by apheresis at Stanford Health Care (SHC) as per current institutional allogeneic transplant protocols.

The first leukapheresis is performed on a continuous flow cell separator according to institutional standards and starts on Day −9±1 day. The second leukapheresis of the donor starts on Day −3±1 day with a target of ≥3×10⁶ CD34+ cells/kg recipient body weight post-selection and a maximum dose of 10×10⁶ CD34+ cells/kg.

In order to have sufficient cells available for CD34 enrichment and Treg sorting procedures, donors either undergo large volume apheresis collection on 1 day (Day −2±1 day) or on 2 consecutive days (Day −3±1); additional apheresis is preformed if needed to ensure a sufficient T-cell count for above stated reasons; an additional mobilizing agent such as plerixafor (Mozobil) is used under these circumstances at the discretion of the attending physician.

At the conclusion of each collection, cellular products are transferred to the cell processing facility. For collection occurring outside of study center, products are transferred on the same day, with the previous days product stored at 4° C. or according to institutional practice but not frozen.

The first day's collection (Day −9±1 day) is kept under refrigeration as a quarantined product in a continuously monitored, secure refrigerator in accordance with apheresis collection storage standards. Prior to being placed in storage, samples for cell count, flow cytometric analysis, and collection sterility testing is aseptically removed by the cell processing facility staff and submitted to the designated group for assessment. After collection, the cellular harvest is transferred via validated transport methods to a gene editing facility.

The 2nd day collection (Day −2±1 day) is transferred to the cell processing facility upon completion of apheresis.

There is a risk of graft failure with allogeneic hematopoietic cell transplant procedures. The donors are notified in the event of graft failure to propose recollection for a second (unmanipulated) transplant procedure.

### Cell Processing

The donor mobilized leukapheresis collections are the source of the 3 infusions products: conventional T-cells (Tcon), regulatory T-cells (Treg), and CD34+ hematopoietic progenitor/stem cells. CD34+ cell enrichment is performed according to procedures given in the CliniMACS Users Operating Manual and institution SOPs. Upon completion of the cell selection process, samples of the CD34-enriched product are aseptically removed for release testing to assess the cellular content, the cell doses and viability, sterility by Gram staining, and endotoxin content, along with additional quality assessments including standard 14-day sterility cultures.

After processing, the cellular components (drug products) are transferred via validated transport methods to dispense the cellular components to the respective subjects.

### Treatment Schedule

The conditioning regimen is one of two options (TABLES 15 and 16).

### First Option:

Doses:
Fractionated total body irradiation (fTBI): 1320 cGy
Cyclophosphamide (Cy): 50 mg/kg×2 doses

TABLE 15
Treatment Schedule I

| Day | Day | Day | Day | Day | Day | Day | Day | Day | Day | Day |
|---|---|---|---|---|---|---|---|---|---|---|
| −8 | −7 | −6 | −5 | −4 | −3 | −2 | −1 | 0 | +1 | +2 |
| fTBI | fTBI | fTBI | fTBI | Cy | Cy | Rest | Rest | CD34+ | Rest | Tcon |
| | | | | | | | | HSPC | | infusion, then |
| | | | | | | | | and Treg | | donor |
| | | | | | | | | infusion | | CD19/CD22- |
| | | | | | | | | | | CAR T |
| | | | | | | | | | | infusion |

### Second Option:

Doses:
Thiotepa: 5 mg/kg×2 doses
Busulfan: 3.2 mg/kg×3 doses
Fludarabine: 60 mg/m², 50 mg/m²×2 doses

TABLE 16
Treatment Schedule II

| Day | Day | Day | Day | Day | Day | Day | Day | Day |
|---|---|---|---|---|---|---|---|---|
| −6 | −5 | −4 | −3 | −2 | −1 | 0 | +1 | +2 |
| Bu/Flu | Bu/Flu | Bu/Flu | TTP | TTP | Rest* | CD34+ | Rest | Tcon |
| | | | | | | HSPC and | | infusion, then |
| | | | | | | Treg infusion | | donor |
| | | | | | | | | CD19/CD22- |
| | | | | | | | | CAR T |
| | | | | | | | | infusion |

*Two days of rest are allowed at the discretion of the treating physician.

Routine anti-emetic prophylaxis and treatment is employed. Corticosteroids are not used (except for physiologic replacement or after discussing with the protocol PI).

Subjects must meet all the following criteria before initiating the conditioning chemotherapy regimen:
Must be at least 14 days, or 5 half-lives, since the last dose of standard chemotherapy, or 7 days, or 5 half-lives, since the last dose of ALL maintenance therapy; with the exception of corticosteroids, which can continue until starting the conditioning chemotherapy regimen on Day −5,
No evidence of uncontrolled infection,
No clinically significant cardiac dysfunction,
Serum creatinine must be <2×ULN,
No acute neurological toxicity >Grade 1 (with the exception of peripheral sensory neuropathy),
Negative pregnancy test within 7 days prior to starting chemotherapy (females of child-bearing potential only) and,
Repeat disease evaluation, at the discretion of the investigator, if the subject received any antineoplastic therapy after enrollment if greater than 28 days has elapsed between date of enrollment and start of conditioning lymphodepletion chemotherapy regimen, or if investigator believes disease may have significantly advanced since enrollment.

Treg CD34+ HSPC infusion: Premedication with acetaminophen (e.g., 500-1000 mg) and diphenhydramine (e.g., 25-50 mg) can be administered prior to administration of each component of the Tcon+Treg+CD34+ HSPC engineered graft, unless contraindicated. Acetaminophen is omitted if necessary or required by institutional guidelines.

Donor CD19/CD22-CAR T cell Infusion: Given the possibility that a subject could develop CRS or neurotoxicity after discharge from the hospital, subjects are asked to remain within commuting distance to the clinic for the first 28 days post CD19/CD22 CAR T cell infusion. Subjects and their family members/caregivers are educated on potential symptoms such as fever, dyspnea, confusion, aphasia, dysphasia, somnolence, encephalopathy, ataxia, or tremor. If subjects develop these symptoms, they are instructed to immediately contact the investigator or seek immediate medical attention.

Subjects must meet the following criteria in order for cells to be infused (based on labs obtained within 24 hours of cell infusion):
CD19/CD22-CAR T cells must have met release criteria,
No evidence of hemodynamic instability,
No new requirement for continuous (>24 hours) supplemental oxygen therapy,
No symptoms concerning for new condition that in the opinion of the PI may pose an unacceptable risk to the subject,
No evidence of clinically significant cardiac dysfunction, uncontrolled, significant tumor lysis syndrome, serum creatinine >2×ULN, and no acute neurological toxicity >Grade 1 (with the exception of peripheral sensory neuropathy).

Corticosteroid use may not exceed >=5 mg/day of prednisone or equivalent for 3 days prior CAR-T infusion.

Other immune suppression medication use is not allowed up to, and during the day of CAR-T cell infusion unless specific exception is granted by the study PI.

All subjects receive levetiracetam (Keppra) 500 mg rally twice a day beginning the day before donor CD19/CD22 CAR T cell infusion. At the onset of ≥Grade 2 neurotoxicity, levetiracetam (Keppra) can be increased from a dose of 500 mg (PO or IV) BID to a dose of 1000 mg (PO or IV) BID. Levetiracetam (Keppra) can continue through day 28 once neurotoxicity develops. This agent is provided by the institution's Pharmacy Department. The package insert can be referred to for complete pharmaceutical information on this product.

Subjects at high risk of TLS, per investigator's discretion, receive allopurinol 300 mg orally once daily beginning the day prior to CD19/CD22 CAR T cell infusion.

Subjects receive the following medications 30-120 minutes prior to cell infusion:
Diphenhydramine 25-50 mg/dose PO or IV;
Acetaminophen 500-1000 mg/dose PO or IV.

### Treatment Dose

The administration of the CD3+ Tcon dose occurs as the first infusion followed by the CD19/CD22-CAR T cell infusion.

At least 3 patients are treated at each dose level (TABLE 1). There is 4 weeks staggering between each patient within a given cohort. The 4-week staggering is from the time of CD19/CD22-CAR T cell infusion to the time of the Tcon+Treg+CD34+ HSPC engineered graft infusion for the next patient. All patients treated at the preceding dose level are observed a minimum of 6 weeks post T cell infusion before dose escalation occurs.

The doses in TABLE 1 are used for the donor derived CD19/CD22CAR T cells (±20%). We employ a Bayesian dose escalation design.

At least 3 patients are treated at the currently selected dose level. There are 4 weeks staggering between each patient within a given cohort. The 4-week staggering is from the time of CD19/CD22-CAR T cell infusion to the time of the Tcon+Treg+CD34+ HSPC engineered graft infusion for the next patient. All patients treated at the preceding dose level are observed a minimum of 6 weeks post T cell infusion before dose escalation occurs.

### GVHD Prophylaxis

All recipients receive single-agent GVHD prophylaxis consisting of single-agent tacrolimus. GVHD prophylaxis begins on the day following donor CD19/CD22-CAR T cells infusion (typically Day +2), starting 12-18 hours after the start of the donor CD19/CD22-CAR T cells infusion.

Tacrolimus is be initiated at 0.015 mg/kg/day for a target range level of 5 to 7 ng/mL. If a trough level of <5 ng/mL is noted prior to the start of tapering of GVHD prophylaxis, tacrolimus dosing is be increased to target the recommended trough level unless clinically contraindicated due to toxicity or after discussion with the PI and/or designee. Per os (PO) administration is permissible when the patient can tolerate food; tacrolimus dosing is initiated via PO route if tolerated.

Tacrolimus taper is initiated at a minimum of 90 days post-HSCT if there is no evidence of active GVHD, with a suggested taper of the prophylaxis regimen by approximately 20% of the dose per month.

Apart from dose modifications, all modifications to the GVHD prophylaxis regimens must be discussed with the Principal Investigator and/or designee, including in cases of intolerance. The reason(s) for modification is be recorded in the CRF.

### Antimicrobial Prophylaxis

All patients receive prophylaxis against bacterial, fungal, and viral infections during the peri-transplant period according to institutional practices.

Of note, all CMV-seropositive recipients receive letermovir prophylaxis beginning between day 0 and day +28; therapy continues through day +100. Letermovir can be omitted for CMV-seropositive recipients only if contraindicated (e.g., known hypersensitivity to letermovir) with PI or PI designee approval.

### Example 2: Tacrolimus Reduces CAR T Cytotoxicity in a Dose-Dependent Manner

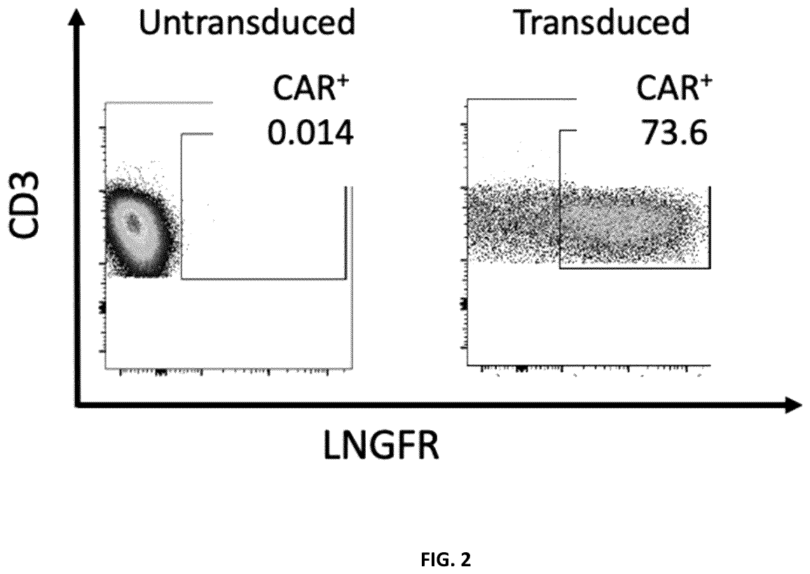

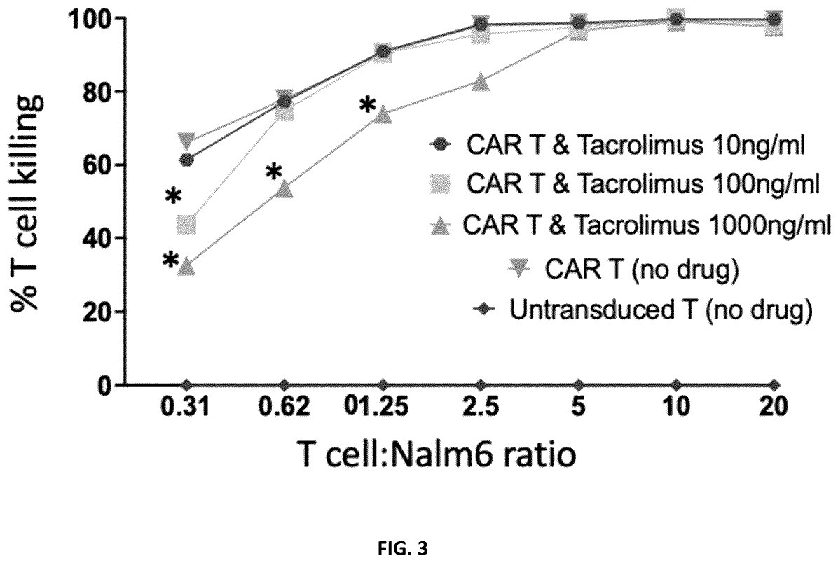

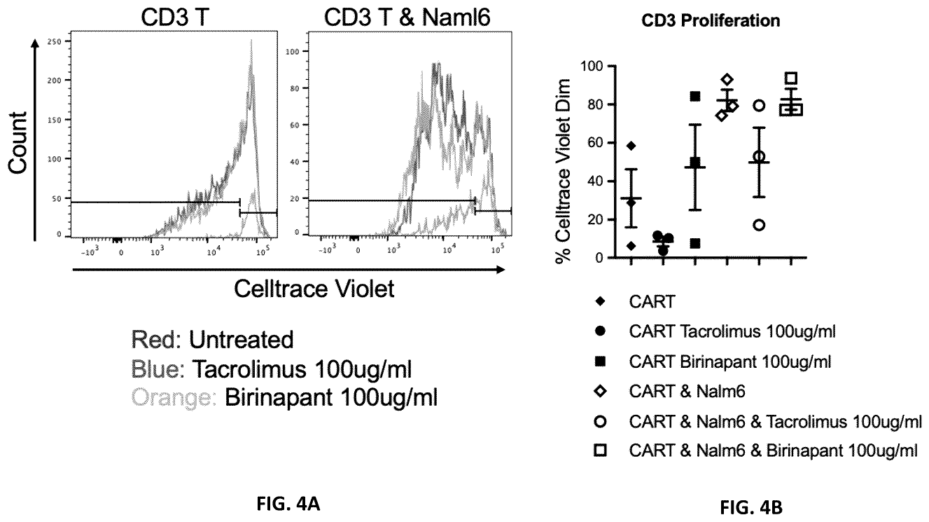

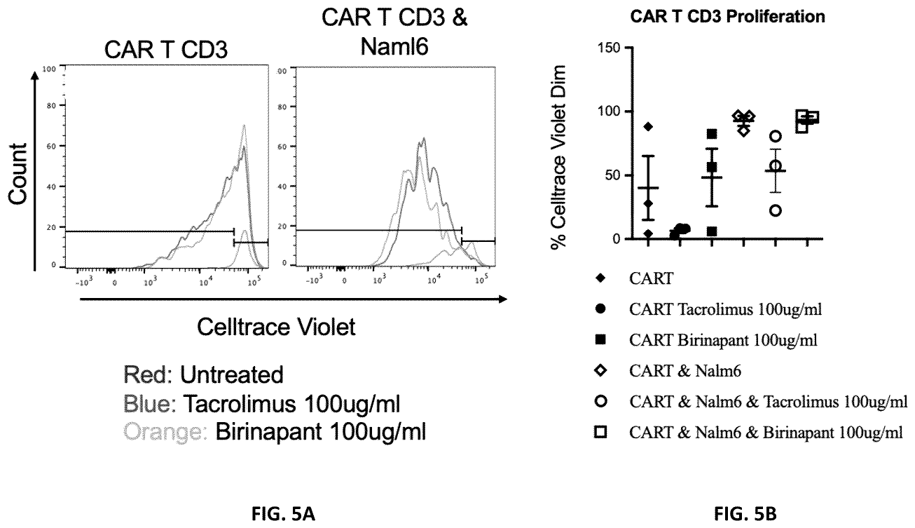

CAR T cells used in the clinical study described in Example 1 were assessed according to standard in vitro methods; CD19 CAR expression was assessed (FIG. 2); CAR T cell cytotoxicity was assessed (FIG. 3); CD3 T cell proliferation was assessed (FIG. 4); and CAR T cell proliferation was assessed (FIG. 4). The results in FIGS. 2-5 indicate that higher concentrations of tacrolimus reduced CAR T cytotoxicity. The results also indicate that higher concentrations of tacrolimus reduced CAR T cell proliferation in the presence or absence of tumor.

### Example 3: Preliminary Clinical Results

Using the treatment protocol described in Example 1, initial subjects were administered the therapy and preliminary results were obtained from two Ph+ B-ALL patients at 3 months and 6 months post treatment (TABLE 17). The first patient (378_001) was evaluated at 6 months, and the second patient (378_0002) was evaluated at 3 months. The status of patient 378_001 at time of treatment was complete response (CR) and measurable residual disease (MRD) negative. The status of patient 378_002 at time of treatment was CR and MRD positive. Engraftment of cell infusion occurred at 16 days post-transplant (D+16) for patient 378_001 and at 14 days post-transplant (D+14) for patient 378_002.

As shown in TABLE 17, neither patient experienced ICANS or GVHD. Both patients had Grade 1 CRS, with patient 378_001 having recurrent Grade 1 CRS. The Grade 3+ adverse events (AEs) were minimal, and expected post-HSPC transplant, for both patients. At time of evaluation, both patients had a CR and were MRD negative.

TABLE 17

| Patient | | | | | Dx |
|---|---|---|---|---|---|
| ID | Gr3 + AEs | CRS | ICANS | GVHD | Status |
| 378_001 | Neutropenia, | Grade 1, | No | None | CR, |
| | Thrombocytopenia, | recurrent | | | MRD− |
| | Anemia | | | | |
| 378_002 | Syncope, Neutro- | Grade 1 | No | None | CR, |
| | penia ,Anorexia, | | | | MRD− |
| | Thrombocytopenia, | | | | |
| | Nausea | | | | |

### Example 4: Further Clinical Study Features

An ongoing clinical study was started with the following features.

### Myeloablative Conditioning (MAC)

Patients that successfully completed the screening procedures and had a match related donor were given a conditioning regimen from approximately 10 to 2 days prior to receiving the T graft and the donor conventional CD19/CD22 CAR-T cell infusion.

The conditioning procedures include one of the study regimens listed below:
Total Body Irradiation, Cyclophosphamide (TBI/Cy)
Busulfan, Fludarabine, and Thiotepa (Bu/Flu/TT)

These conditioning regimens were administered over the course of approximately 6-8 days. Adverse events were monitored. Adverse events are defined as any unfavorable and unintended sign or symptom, including abnormal laboratory findings, or disease, that is temporally associated with the use of a drug.

### Cell Infusions

### CD34+ HSPC Treg Infusion

### Treatment (Day 0 and Day 2)

On day 0, the day of transplant, the following procedures were performed:
Physical exam, including weight
Vital signs
Performance score
Blood tests
All cytomegalovirus-seropositive recipients received letermovir prophylaxis beginning between day 0 and day +28; therapy continued through day +100. Other subjects received letermovir per institutional practice.
A combination of stem cells and Treg were infused through central venous catheter.

On day 1, the day between infusions, the following procedures were performed:
Premedications
   Keppra, an antiseizure medication
   Participants that were high-risk for tumor lysis syndrome (TLS), per the Protocol Director's or Sub-Investigator's discretion, received allopurinol (uric acid reducer) orally once daily
Physical exam, including weight
Vital signs
Neurologic examination
Blood tests and laboratory tests
Brain MRI or other appropriate imaging, if it was necessary, based on disease location in the body

On day 2, the following procedures were performed:
Premedications
   Keppra, an antiseizure medication
Physical exam, including weight
Vital signs
Blood tests.
The Tcon cells were infused through the central venous catheter prior infusion of CD19/CD22 CAR-T cells
The CD19/CD22-CAR T cells are given intravenously over 10-30 minutes. Participants were closely monitored (taking your temperature, blood pressure, heart rate and breathing rate) during and after the infusion and treated immediately if side effects were observed.

Participants were monitored for adverse events throughout the time period.

### Post-Transplant Assessment Period

Participants were given daily safety tests from Day 0 to Day 21, then twice per week until Day 30. The following assessments and procedures were also performed:
Brief Physical exam
Neurological exam
Vital signs and a questionnaire designed to test mental status and ability to complete tasks (ICE assessment), performed every 12 hours while the participants were still in the hospital and then during clinic visits until Day 21
Patients were given tacrolimus to prevent the development of GVHD, starting on Day +3. Typically, it was given IV first, but later be changed to an oral route in the case of participants able to tolerate food. Tacrolimus administration was continued until at least Day +60 after transplant, at which time the dose was slowly tapered under doctor guidance.
GVHD assessment
Viral testing to for active Epstein-Barr Virus (EBV) and/or cytomegalovirus (CMV) infections.
Keppra, an antiseizure medication
All cytomegalovirus-seropositive recipients received letermovir prophylaxis beginning between day 0 and day 28; therapy continued through day 100. Some subjects may have received letermovir per institutional practice.
Blood sample collection for routine and research testing.

### Example 5: Further Clinical Results

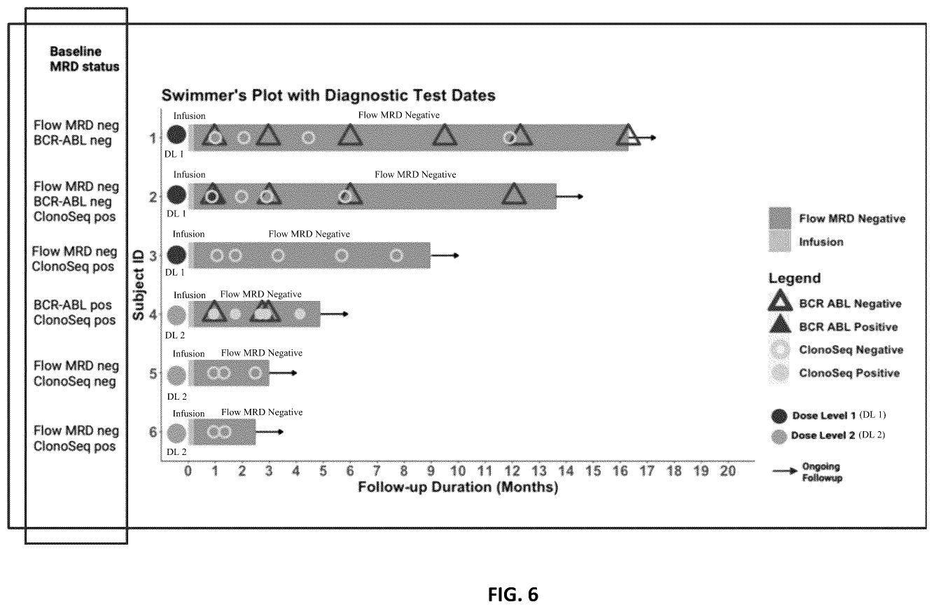

Using the treatment protocol described in Example 1, additional subjects were administered the therapy and results were obtained from six patients at the months indicated in FIG. 6. Patient baseline characteristics are described in TABLE 18.

TABLE 18

| Baseline Characteristics | N = 6 | |
|---|---|---|
| Age (median, range) | 26.5 | (21-37) |
| Female | 4 | (67%) |
| Hispanic | 5 | (83%) |
| High Risk Feature: | | |
| Ph-like | 2 | (33%) |
| MRD+ post-induction | 6 | (100%) |
| Relapsed | 1 | (17%) |
| ALL Status at HCT: | | |
| CR MRD− | 2 | (33%) |
| CR MRD+ | 3 | (50%) |
| Active Disease | 1 | (17%) |
| HLA Matched Sib Donor | 6 | (100%) |
| TBI/CY Conditioning | 6 | (100%) |
| CAR T Dose Level: | | |
| DL 1 | 3 | (50%) |
| DL2 | 3 | (50%) |

Safety and efficacy outcomes of the six patients are depicted in TABLE 19 and FIG. 6.

TABLE 19

| Safety and Efficacy | N = 6 |
|---|---|
| Engraftment: | |
| Median Days - Neutrophil | 14 |
| Median Days - Platelet | 15 |
| Primary Graft Failure | 0 |
| GVHD: | |
| Acute GVHD | 0 |
| Chronic GVHD | 0 |
| CRS: | |
| Grade 1 | 6 (100%) |
| ICANS | 0 |
| Day 42 Response: | |
| MRD− | 5 (80%) |
| MRD+ | 1 (20%) |

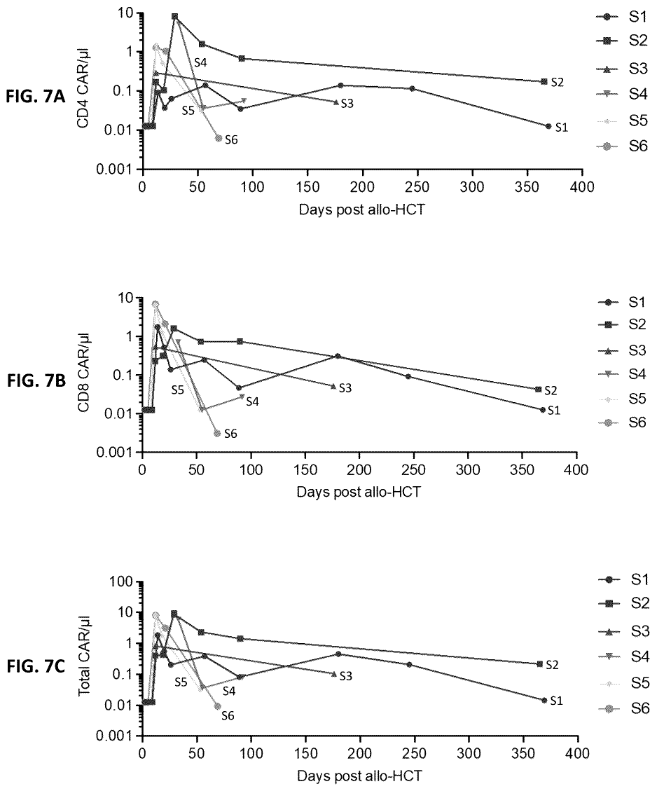

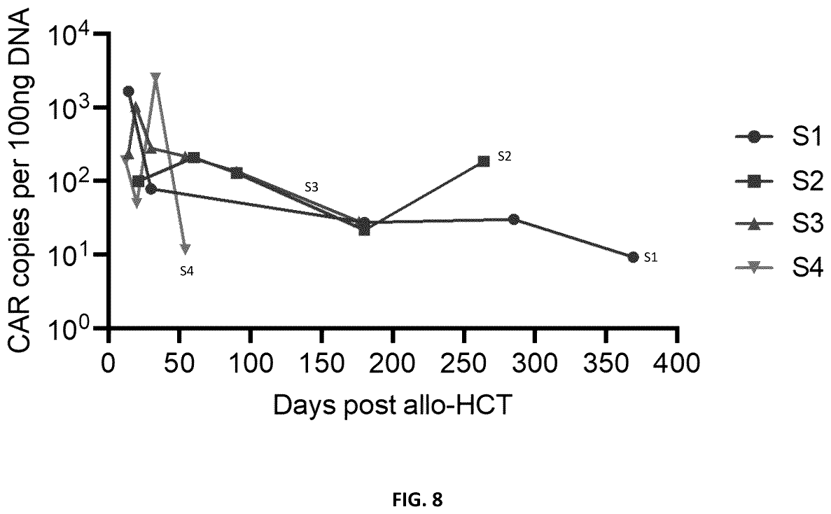

The absolute number of circulating CD4⁺ CD19/22.BB.z CAR T cells (FIG. 7A), CD8⁺ CD19/22.BB.z CAR T cells (FIG. 7B), and total CD19/22.BB.z CAR T cells (FIG. 7C) after infusion into patients was measured by flow cytometry (FACS). The number of circulating CD19/22.BB.z CAR T cell copies per 100 ng of genomic DNA in patients was also measured by qPCR (FIG. 8). The results in FIG. 8 show the initial expansion and persistence of the donor CD19/22 CAR T cells.

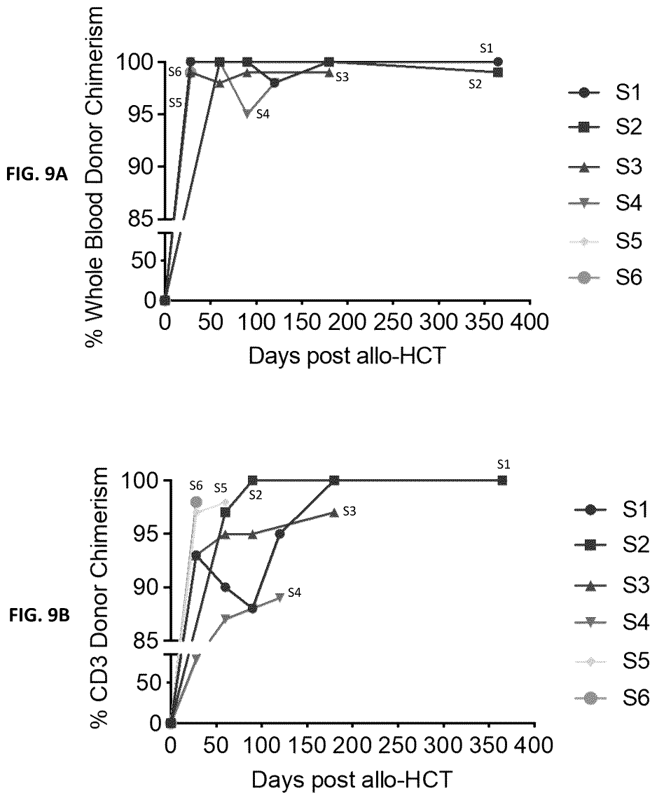

The percent chimerism of whole blood (FIG. 9A) and CD3⁺ cells (FIG. 9B) was also measured after infusion of the therapy into the patients.

The results from the six patients treated with the therapy indicate that all 6 patients experienced prompt donor engraftment, and all achieved flow/BCR-ABL MRD-negative CR (one patient remains positive by NGS only). In addition, to date none of the 6 patients have developed acute or chronic GVHD. Further, the results in FIG. 7 and FIG. 8 demonstrate the persistence of donor CD19/22 CAR T cells beyond 1 year.

### Example 6: Additional Clinical Results

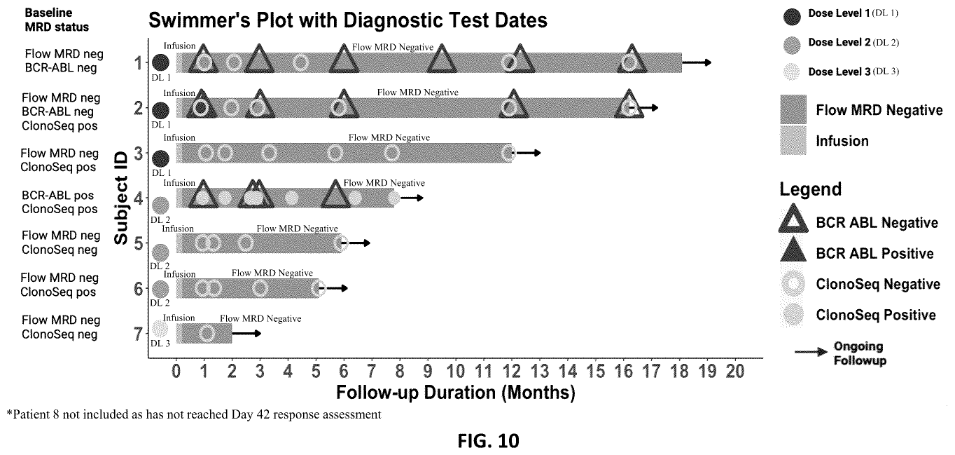

Using the treatment protocol described in Example 1, additional subjects were administered the therapy and results were obtained from eight patients at the months indicated in FIG. 10.

The median age of the treated patients was 28 (range, 21-52). Six patients (75%) were Hispanic. Regarding high-risk disease features, two patients had Ph-like ALL, one had TP53-mutated ALL, seven were MRD-positive post-induction, and one had relapsed disease. At the time of enrollment in the study, 3 patients (37%) were in an MRD-negative CR, 4 patients (50%) were in an MRD-positive CR, and 1 patient (13%) had active disease. Seven patients (88%) received an allograft from an HLA matched sibling donor, while 1 patient (12%) received an allograft from a matched unrelated donor.

Safety and efficacy outcomes of the six patients are depicted in TABLE 20.

TABLE 20

| Safety and Efficacy | N = 8 | |
|---|---|---|
| Engraftment | | |
| Median Days- Neutrophil | 14 | |
| Median Days- Platelet | 11 | |
| Primary Graft Failure | 0 | |
| GVHD | | |
| Acute GVHD | 0 | |
| Chronic GVHD | 0 | |
| CRS | | |
| Grade 1 | 7 | (88%) 1 |
| Grade 2 | | (12%) |
| ICANS | 0 | |
| Day 42 Response* | | |
| MRD− | 6 | (86%) |
| MRD+ | 1 | (14%) |

*Patient 8 not included as has not reached Day 42 response assessment

At Day +42, post-treatment assessment, all patients demonstrated an MRD-negative CR as measured by flow cytometry and BCR::ABL1 PCR (Ph+ ALL, while seven out of eight patients were MRD-negative by NGS (conoSEQ™). With a median follow-up of 190 days (range, 78-502, including 6 patients with >6 months of follow-up), all patients have an ongoing MRD-negative CR. At last assessment, whole blood and CD3 chimerism exceeds 95% in all patients.

All patients had good expansion of allogeneic CAR in the presence of immunosuppression with tacrolimus. Although the assessment of CAR T cell persistence is ongoing, 3/3 patients, all of whom were treated on DL1, have allogeneic CAR T cells detectable beyond one year.

What is claimed is:

1. A multi-component cellular therapy product comprising:
(a) one or more cells comprising a chimeric receptor or a pharmaceutical composition comprising an isolated nucleic acid encoding the chimeric receptor; and
(b) an immune-modulating cell therapy product,
optionally wherein the one or more cells comprising the chimeric receptor or the pharmaceutical composition and/or one or more cells of the immune-modulating cell therapy product are allogeneic cells from an HLA-compatible donor, relative to a human subject receiving the multi-component cellular therapy product.

2. The multi-component cellular therapy product of claim 1, wherein:
a) the HLA-compatible donor is an allogeneic HLA-matched donor, relative to the human subject, or
the HLA-compatible donor is an allogeneic HLA-mismatched donor, relative to the human subject; and/or
b) the one or more allogeneic cells have not been genetically modified to reduce risk of graft versus host disease (GVHD) and/or to prevent allogeneic rejection with respect to the human subject; and/or
c) the immune-modulating cell therapy product comprises a regulatory T cell therapy, or the immune-modulating cell therapy product comprises an allogeneic hematopoietic stem cell transplant (HSCT),
optionally wherein the allogeneic HSCT comprises a population of CD34⁺ hematopoietic stem and progenitor cells (HSPCs), or the allogeneic HSCT comprises a population of CD4⁺CD25⁺CD127^{dim} regulatory T cells (Tregs), or the allogeneic HSCT comprises HSPCs and Tregs, and/or
optionally wherein the allogeneic HSCT comprises a population of CD45RA⁻ memory T cells (Tmems), or the allogeneic HSCT comprises a population of HSPCs and a population of Tmems, or the allogeneic HSCT comprises a population of Treg and a population of Tmems, or the allogeneic HSCT comprises a population of HSPCs, a population of Treg, and a population of Tmems, and/or
optionally wherein the allogeneic HSCT has been depleted of naïve conventional CD3⁺CD25⁻ CD45RA⁺ T cells, and/or
optionally wherein the allogeneic HSCT comprises a population of conventional CD3⁺ T cells (Tcons), or the allogeneic HSCT comprises a population of HSPCs and a population of Tcons, or the allogeneic HSCT comprises a population of Tregs and a population of Tcons, or the allogeneic HSCT comprises a population of Tmems and a population of Tcons, or the allogeneic HSCT comprises a population of HSPCs, a population of Tregs, and a population of Tcons, or the allogeneic HSCT comprises a population of HSPCs, a population of Tregs, a population of Tmems, and a population of Tcons, and/or
optionally wherein the allogeneic HSCT is a T cell-depleted allogeneic HSCT; and/or
d) the immune-modulating cell therapy product comprises a population of CD34⁺ hematopoietic stem and progenitor cells (HSPCs), optionally wherein the population of HSPCs comprises the one or more cells comprising the chimeric receptor or the pharmaceutical composition,
optionally wherein the immune-modulating cell therapy product further comprises a population of CD4⁺CD25⁺CD127^{dim} regulatory T cells (Tregs), optionally wherein the population of Tregs comprises the one or more cells comprising the chimeric receptor or the pharmaceutical composition,
and/or the immune-modulating cell therapy product further comprises a population of CD45RA⁻ memory T cells (Tmems), optionally wherein the population of Tmems comprises the one or more cells comprising the chimeric receptor or the pharmaceutical composition, and/or the immune-modulating cell therapy product further comprises a population of conventional CD3⁺ T cells (Tcons), optionally wherein the population of Tcons comprises the one or more cells comprising the chimeric receptor or the pharmaceutical composition; and or
e) the immune-modulating cell therapy product comprises a population of CD4⁺CD25⁺CD127^{dim} regulatory T cells (Tregs), optionally wherein the population of Tregs comprises the one or more cells comprising the chimeric receptor or the pharmaceutical composition,
optionally wherein the immune-modulating cell therapy product further comprises a population of CD34⁺ hematopoietic stem and progenitor cells (HSPCs), optionally wherein the population of HSPCs comprises the one or more cells comprising the chimeric receptor or the pharmaceutical composition,
and/or the immune-modulating cell therapy product further comprises a population of CD45RA⁻ memory T cells (Tmems), optionally wherein the population of Tmems comprises the one or more cells comprising the chimeric receptor or the pharmaceutical composition, and/or the immune-modulating cell therapy product further comprises a population of conventional CD3⁺ T cells (Tcons), optionally wherein the population of Tcons comprises the one or more cells comprising the chimeric receptor or the pharmaceutical composition; and/or
f) the immune-modulating cell therapy product comprises a population of CD45RA⁻ memory T cells (Tmems), optionally wherein the population of Tmems comprises the one or more cells comprising the chimeric receptor or the pharmaceutical composition,
optionally wherein the immune-modulating cell therapy product further comprises a population of CD34⁺ hematopoietic stem and progenitor cells (HSPCs), optionally wherein the population of HSPCs comprises the one or more cells comprising the chimeric receptor or the pharmaceutical composition,
and/or the immune-modulating cell therapy product further comprises a population of CD4⁺CD25⁺CD127^{dim} regulatory T cells (Tregs), optionally wherein the population of Tregs comprises the one or more cells comprising the chimeric receptor or the pharmaceutical composition,
and/or the immune-modulating cell therapy product further comprises a population of conventional CD3⁺ T cells (Tcons), optionally wherein the population of Tcons comprises the one or more cells comprising the chimeric receptor or the pharmaceutical composition; and/or
g) the immune-modulating cell therapy product comprises a population of conventional CD3⁺ T cells (Tcons), optionally wherein the population of Tcons comprises the one or more cells comprising a chimeric receptor
optionally wherein the immune-modulating cell therapy product further comprises a population of CD34⁺ hematopoietic stem and progenitor cells (HSPCs), optionally wherein the population of HSPCs comprises the one or more cells comprising the chimeric receptor or the pharmaceutical composition,
and/or the immune-modulating cell therapy product further comprises a population of CD4⁺CD25⁺CD127^{dim} regulatory T cells (Tregs), optionally wherein the population of Tregs comprises the one or more cells comprising the chimeric receptor or the pharmaceutical composition,
and/or the immune-modulating cell therapy product further comprises a population of CD45RA⁻ memory T cells (Tmems), optionally wherein the population of Tmems comprises the one or more cells comprising the chimeric receptor or the pharmaceutical composition; and/or
h) the immune-modulating cell therapy product further comprises a population of tumor-infiltrating lymphocytes (TILs), optionally wherein the population of Tcons comprises the population of TILs.

3. The multi-component cellular therapy product of claim 1 or claim 2, wherein the pharmaceutical composition comprises a vector comprising the nucleic acid, optionally wherein the vector is a viral vector, optionally wherein the viral vector is selected from the group consisting of: a lentiviral vector, a retroviral vector, an oncolytic viral vector, an adenoviral vector, an adeno-associated viral (AAV) vector, and a virus-like particle (VLP), or
the pharmaceutical composition comprises a nanoparticle comprising the nucleic acid, optionally wherein the nanoparticle is a lipid nanoparticle (LNP),
optionally wherein the pharmaceutical composition further comprises a pharmaceutically acceptable carrier, pharmaceutically acceptable excipient, or a combination thereof.

4. The multi-component cellular therapy product of any one of claims 1-3, wherein:
a) the Tcons are formulated for administration approximately 12 hours or more after administration of the HSPCs, after administration of the Tregs, after administration of the Tmems, or after administration of any combination of the HSPCs, the Tregs, and the Tmems, or the Tcons are formulated for administration from approximately 24 to approximately 120 hours after administration of the HSPCs, after administration of the Tregs, after administration of the Tmems, or after administration of any combination of the HSPCs, the Tregs, and the Tmems, or the Tcons are formulated for administration from approximately 36 to approximately 72 hours after administration of the HSPCs, after administration of the Tregs, after administration of the Tmems, or after administration of any combination of the HSPCs, the Tregs, and the Tmems; and/or
b) the Tregs, the Tmems, or the Tregs and the Tmems are formulated for administration from approximately 5 minutes to approximately 5 hours after administration of the HSPCs; and/or
c) the one or more cells comprising the chimeric receptor or the pharmaceutical composition are formulated for administration approximately 12 hours or more after administration of the HSPCs, after administration of the Tregs, after administration of the Tmems, or after administration of any combination of the HSPCs, the Tregs, and the Tmems, or
the one or more cells comprising the chimeric receptor or the pharmaceutical composition are formulated for administration from approximately 24 to approximately 120 hours after administration of the HSPCs, after administration of the Tregs, after administration of the Tmems, or after administration of any combination of the HSPCs, the Tregs, and the Tmems, or
the one or more cells comprising the chimeric receptor or the pharmaceutical composition are formulated for administration from approximately 36 to approximately 72 hours after administration of the HSPCs, after administration of the Tregs, after administration of the Tmems, or after administration of any combination of the HSPCs, the Tregs, and the Tmems, or
the one or more cells comprising the chimeric receptor or the pharmaceutical composition are formulated for administration from approximately 5 minutes to approximately 5 hours after administration of the Tcons.

5. The multi-component cellular therapy product of any one of claims 1-4, wherein:
a) the multi-component cellular therapy product further comprises a pharmaceutical composition comprising one or more doses of a graft vs host disease (GVHD) prophylactic agent, optionally wherein the GVHD prophylactic agent comprises tacrolimus; and/or
b) the multi-component cellular therapy product comprises a dose of HSPCs that ranges from approximately 1.0×10⁵ to approximately 1.0×10⁸ HSPCs per kilogram of body weight of a human subject receiving the product, or from approximately 5.0×10⁵ to approximately 1.5×10¹⁰ HSPCs; and/or
c) the multi-component cellular therapy product comprises a dose of Tregs that ranges from approximately 1.0×10⁵ to approximately 2.0×10⁷ Tregs per kilogram of body weight of a human subject receiving the product, or from approximately 5.0×10⁵ to approximately 3.0×10⁹ Tregs, optionally wherein the Tregs are FOXP3⁺; and/or
d) the multi-component cellular therapy product comprises a dose of Tmems that ranges from approximately 1.0×10⁵ to approximately 1.0×10⁸ Tmems per kilogram of body weight of a human subject receiving the product, or from approximately 5.0×10⁵ to approximately 1.5×10¹⁰ Tmems; and/or
e) the multi-component cellular therapy product comprises a dose of Tcons that ranges from approximately 1.0×10⁵ to approximately 4.0×10⁷ Tcons per kilogram of body weight of a human subject receiving the product, or from approximately 5.0×10⁵ to approximately 6.0×10⁹ Tcons.

6. The multi-component cellular therapy product of any one of claims 1-5, wherein:
a) the one or more cells comprising the chimeric receptor or the pharmaceutical composition comprise one or more genetic modifications that increase immune tolerance with respect to a human subject receiving the product; and/or
b) the one or more cells comprising the chimeric receptor or the pharmaceutical composition have not been modified to increase immune tolerance with respect to a human subject receiving the product; and/or
c) the one or more cells comprising the chimeric receptor or the pharmaceutical composition are donor-derived from an allogeneic donor with respect to a human subject receiving the product; and/or
d) the HSPCs, the Tregs, the Tmems, the Tcons, or any combination thereof are donor-isolated,
optionally wherein the donor is allogeneic with respect to a human subject receiving the product; and/or
e) the HSPCs, the Tregs, the Tmems, the Tcons, or any combination thereof, and the one or more cells comprising the chimeric receptor or the pharmaceutical composition were derived from the same allogeneic donor,
optionally wherein the allogeneic donor is HLA-matched, relative to a human subject receiving the product, at one or more alleles selected from the group consisting of: HLA-A, HLA-B, HLA-C, and HLA-DRB1,
optionally wherein the allogeneic donor is 8/8 HLA-matched, relative to a human subject receiving the product, for alleles HLA-A, HLA-B, HLA-C, and HLA-DRB1,
optionally wherein the allogeneic donor has at least one HLA mismatch, relative to a human subject receiving the product, optionally wherein the at least one HLA mismatch is at an allele selected from the group consisting of: HLA-A, HLA-B, HLA-C, HLA-DRB1, and any combination thereof,
optionally wherein the allogeneic donor is an allogeneic haploidentical donor, relative to a human subject receiving the product, optionally wherein the allogeneic haploidentical donor is at least 4/8 HLA-mismatched, at least 5/8 HLA-mismatched, or at least 6/8 HLA-mismatched at an allele selected from the group consisting of: HLA-A, HLA-B, HLA-C, HLA-DRB1, and any combination thereof; and/or
f) the HSPCs, the Tregs, the Tmems, the Tcons, or any combination thereof, and/or the one or more cells comprising the chimeric receptor or the pharmaceutical composition were collected from one or more cord blood samples from the donor, or were collected from one or more bone marrow samples from the donor, or were collected from two or more peripheral blood samples from the donor, optionally wherein at least one of the two or more samples are mobilized samples, optionally wherein the HSPCs, the Tregs, the Tmems, the Tcons, or any combination thereof are obtainable from the mobilized samples, optionally wherein at least one mobilized sample is mobilizable by granulocyte colony-stimulating factor (G-CSF), granulocyte macrophage colony-stimulating factor (GM-CSF), mozobil, or any combination thereof.

7. The multi-component cellular therapy product of any one of claims 1-6, further comprising a conditioning regimen, wherein the conditioning regimen is formulated for administration before administration of the cellular therapy,
optionally wherein the conditioning regimen is formulated for administration from approximately two days to approximately ten days before administration of the multi-component cellular therapy,
optionally wherein the conditioning regimen is a total body irradiation-based (TBI-based) regimen or a total marrow and lymphoid irradiation-based (TMLI-based) regimen,
optionally wherein the TBI-based regimen comprises fractionated total body irradiation (fTBI),
optionally wherein the fTBI comprises 2 or more, 3 or more, 4 or more, 5 or more, 6 or more, 7 or more, 8 or more, 9 or more, or 10 or more doses,
optionally wherein the fTBI comprises a total dose that ranges from approximately 500 to approximately 1600 cGy,
optionally wherein the TBI-based or TMLI-based regimen further comprises one or more doses of a conditioning reagent, optionally wherein the conditioning reagent comprises one or more conditioning reagents selected from the group consisting of: cyclophosphamide, etoposide, thiotepa, busulfan, melphalan, fludarabine, and any combination thereof,
optionally wherein the conditioning regimen is a myeloablative conditioning regimen or a reduced-intensity conditioning (RIC) regimen, optionally wherein the myeloablative conditioning regimen or RIC regimen comprises one or more conditioning reagents, optionally wherein the one or more conditioning reagents are selected from the group consisting of: thiotepa, busulfan, melphalan, fludarabine, cyclophosphamide, anti-thymocyte globulin (ATG), and any combination thereof,
optionally wherein the myeloablative conditioning regimen comprises three or more conditioning reagents, wherein at least one conditioning reagent comprises thiotepa, optionally wherein the myeloablative conditioning regimen comprises one or more doses of busulfan, one or more doses of fludarabine, and one or more doses of thiotepa.

8. The multi-component cellular therapy product of any one of claims 1-7, wherein:
a) the chimeric receptor comprises one or more extracellular antigen-binding domains that bind to one or more antigens selected from the group consisting of: BCMA, PSMA, CD8, CD10, CD19, CD20, CD21, CD22, CD25, CD30, CD33, CD34, CD37, CD44v6, CD45, CD52, CSF1R (CD115), c-Kit (CD117), CD123, CD133, Fms-like tyrosine kinase 3 (FLT-3, CD135), PDGFR-alpha (CD140a), PDGFR-beta (CD140b), CD186, CD195, VEGFR3 (FLT4, CD309), GPRC5D, TMEFF2, ROR1, B7-H6, B7-H3, HM1.24, SLAMF7, chondroitin sulfate proteoglycan 4 (CSPG4, melanoma-associated chondroitin sulfate proteoglycan), epidermal growth factor receptor (EGFR), Her2, Her3, IGFR, IL3R, fibroblast activating protein (FAP), CDCP1, Derlin1, Tenascin, frizzled 1-10, VEGFR2 (KDR/FLK1), endoglin, CLEC14, Tem1-8, Tie2, A33, CAMPATH-1 (CDw52), CEA, Carbonic anhydrase IX (MN/CA IX), de2-7, EGFRvIII, EpCAM, Ep-CAM, folate-binding protein, G250, HLA-DR, IGFR, IL-2 receptor, IL3R, MCSP (melanoma-associated cell surface chondroitin sulphate proteoglycane), Muc-1, prostate stem cell antigen (PSCA), prostate specific antigen (PSA), hK2, TAG-72, KI2L4, NKG2E, NKG2D, NKG2F, BTNL3, BTNL8, PD-1, NKG2, a tumor cell neoantigen, and any combination thereof; and/or
b) the chimeric receptor is a chimeric antigen receptor (CAR) or T cell receptor (TCR).

9. The multi-component cellular therapy product of any one of claims 1-8, wherein:
a) the chimeric receptor is a chimeric antigen receptor (CAR), and the CAR further comprises a transmembrane domain and one or more intracellular signaling domains; and/or
b) the transmembrane domain is selected from the group consisting of: a CD8 transmembrane domain, a CD28 transmembrane domain a CD3zeta-chain transmembrane domain, a CD4 transmembrane domain, a 4-IBB transmembrane domain, an 0X40 transmembrane domain, an ICOS transmembrane domain, a CTLA-4 transmembrane domain, aPD-1 transmembrane domain, a LAG-3 transmembrane domain, a 2B4 transmembrane domain, a BTLA transmembrane domain, an 0X40 transmembrane domain, a DAP 10 transmembrane domain, a DAP 12 transmembrane domain, a CD16a transmembrane domain, a DNAM-1 transmembrane domain, a KIR2DSl transmembrane domain, aKIR3DSl transmembrane domain, an NKp44 transmembrane domain, an NKp46 transmembrane domain, an FceRlg transmembrane domain, and an NKG2D transmembrane domain; and/or
c) the one or more intracellular signaling domains are each selected from the group consisting of: a CD3zeta-chain intracellular signaling domain, a CD97 intracellular signaling domain, a CD1 la-CD18 intracellular signaling domain, a CD2 intracellular signaling domain, an ICOS intracellular signaling domain, a CD27 intracellular signaling domain, a CD154 intracellular signaling domain, a CDS intracellular signaling domain, an 0X40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP 10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceRlg intracellular signaling domain, a NKG2D intracellular signaling domain, and an EAT-2 intracellular signaling domain.

10. The multi-component cellular therapy product of any one of claims 1-9, wherein:
a) the CAR is a bispecific CAR that comprises two or more extracellular antigen-binding domains, optionally wherein the CAR comprises a first antigen-binding domain specific for CD22 and a second antigen-binding domain specific for CD19,
optionally wherein the first antigen-binding domain comprises a heavy chain variable (VH) region and a light chain variable (VL) region, wherein the VH comprises: a heavy chain complementarity determining region 1 (CDR-H1) having the amino acid sequence of GDSVSSNSAA (SEQ ID NO: 1), a heavy chain complementarity determining region 2 (CDR-H2) having the amino acid sequence of TYYRSKWYN (SEQ ID NO: 2), and a heavy chain complementarity determining region 3 (CDR-H3) having the amino acid sequence of AREVTGDLEDAFDI (SEQ ID NO: 3), and wherein the VL comprises: a light chain complementarity determining region 1 (CDR-L1) having the amino acid sequence of QTIWSY (SEQ ID NO: 4), a light chain complementarity determining region 2 (CDR-L2) having the amino acid sequence of AAS (SEQ ID NO: 5), and a light chain complementarity determining region 3 (CDR-L3) having the amino acid sequence of QQSYSIPQT (SEQ ID NO: 6),
optionally wherein the second antigen-binding domain comprises a heavy chain variable (VH) region and a light chain variable (VL) region, wherein the VH comprises: a heavy chain complementarity determining region 1 (CDR-H1) having the amino acid sequence of GVSLPDYG (SEQ ID NO: 7), a heavy chain complementarity determining region 2 (CDR-H2) having the amino acid sequence of IWGSETT (SEQ ID NO: 8), and a heavy chain complementarity determining region 3 (CDR-H3) having the amino acid sequence of AKHYYYGGSYAMDY (SEQ ID NO: 9), and wherein the VL comprises: a light chain complementarity determining region 1 (CDR-L1) having the amino acid sequence of QDISKY (SEQ ID NO: 10), a light chain complementarity determining region 2 (CDR-L2) having the amino acid sequence of HTS (SEQ ID NO: 11), and a light chain complementarity determining region 3 (CDR-L3) having the amino acid sequence of QQGNTLPYT (SEQ ID NO: 12),
optionally wherein the first antigen-binding domain comprises a VH having amino acid sequence of
(SEQ ID NO: 13)
QVQLQQSGPGLVKPSQTLSLTCAISGDSVSSNSAAWNWIRQSPSRGLEW
LGRTYYRSKWYNDYAVSVKSRITINPDTSKNQFSLQLNSVTPEDTAVYY
CAREVTGDLEDAFDIWGQGTMVTVSS,
optionally wherein the first antigen-binding domain comprises a VL having amino acid sequence of
(SEQ ID NO: 14)
DIQMTQSPSSLSASVGDRVTITCRASQTIWSYLNWYQQRPGKAPNLLIY
AASSLQSGVPSRFSGRGSGTDFTLTISSLQAEDFATYYCQQSYSIPQTF
GQGTKLEIK,
optionally wherein the second antigen-binding domain comprises a VH having amino acid sequence of
(SEQ ID NO: 15)
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSS,
optionally wherein the second antigen-binding domain comprises a VL having the amino acid sequence of
(SEQ ID NO: 16)
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEIT,
optionally wherein the first antigen-binding domain comprises a CD22 scFv having the amino acid sequence of:
```
  (SEQ ID NO: 20)
QVQLQQSGPGLVKPSQTLSLTCAISGDSVSSNSAAWNWIRQSPSRGLEW
LGRTYYRSKWYNDYAVSVKSRITINPDTSKNQFSLQLNSVTPEDTAVYY
CAREVTGDLEDAFDIWGQGTMVTVSSGSTSGSGKPGSGEGSTKGDIQMT
QSPSSLSASVGDRVTITCRASQTIWSYLNWYQQRPGKAPNLLIYAASSL
QSGVPSRFSGRGSGTDFTLTISSLQAEDFATYYCQQSYSIPQTFGQGTK
LEIK
or
  (SEQ ID NO: 21)
QVQLQQSGPGLVKPSQTLSLTCAISGDSVSSNSAAWNWIRQSPSRGLEW
LGRTYYRSKWYNDYAVSVKSRITINPDTSKNQFSLQLNSVTPEDTAVYY
CAREVTGDLEDAFDIWGQGTMVTVSSGGGGSDIQMTQSPSSLSASVGDR
VTITCRASQTIWSYLNWYQQRPGKAPNLLIYAASSLQSGVPSRFSGRGS
GTDFTLTISSLQAEDFATYYCQQSYSIPQTFGQGTKLEIK,
```
optionally wherein the second antigen-binding domain comprises a CD19 scFv having the amino acid sequence of:
(SEQ ID NO: 22)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITGSTSGSGKPGSGEGSTKGEVKLQESGPGLVAPSQSLSVTC
TVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSS
or
(SEQ ID NO: 41)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITGGGGSEVKLQESGPGLVAPSQSLSVTCTVSGVSLPDYGVS
WIRQPPRKGLEWLGVIWGSETTYYNSALKSRLTIIKDNSKSQVFLKMNS
LQTDDTAIYYCAKHYYYGGSYAMDYWGQGTSVTVSS
and/or
b) wherein the CAR comprises the amino acid sequence of:
```
i.
  (SEQ ID NO: 23)
DIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGG
SQVQLQQSGPGLVKPSQTLSLTCAISGDSVSSNSAAWNWIRQSPSRGLEWLGRTY
YRSKWYNDYAVSVKSRITINPDTSKNQFSLQLNSVTPEDTAVYYCAREVTGDLE
DAFDIWGQGTMVTVSSGSTSGSGKPGSGEGSTKGDIQMTQSPSSLSASVGDRVTI
TCRASQTIWSYLNWYQQRPGKAPNLLIYAASSLQSGVPSRFSGRGSGTDFTLTISS
LQAEDFATYYCQQSYSIPQTFGQGTKLEIKGGGGSEVKLQESGPGLVAPSQSLSV
TCTVSGVSLPDYGVSWIRQPPRKGLEWLGVIWGSETTYYNSALKSRLTIIKDNSK
SQVFLKMNSLQTDDTAIYYCAKHYYYGGSYAMDYWGQGTSVTVSSS,
ii.
  (SEQ ID NO: 24)
QVQLQQSGPGLVKPSQTLSLTCAISGDSVSSNSAAWNWIRQSPSRGLEWLGRTY
YRSKWYNDYAVSVKSRITINPDTSKNQFSLQLNSVTPEDTAVYYCAREVTGDLE
DAFDIWGQGTMVTVSSGGGGSDIQMTQSPSSLSASVGDRVTITCRASQTIWSYLN
WYQQRPGKAPNLLIYAASSLQSGVPSRFSGRGSGTDFTLTISSLQAEDFATYYCQ
QSYSIPQTFGQGTKLEIKGGGGSGGGGSGGGGSGGGGSGGGGSDIQMTQTTSSLS
ASLGDRVTISCRASQDISKYLNWYQQKPDGTVKLLIYHTSRLHSGVPSRFSGSGS
GTDYSLTISNLEQEDIATYFCQQGNTLPYTFGGGTKLEITGSTSGSGKPGSGEGST
KGEVKLQESGPGLVAPSQSLSVTCTVSGVSLPDYGVSWIRQPPRKGLEWLGVIW
GSETTYYNSALKSRLTIIKDNSKSQVFLKMNSLQTDDTAIYYCAKHYYYGGSYA
MDYWGQGTSVTVSS
iii.
  (SEQ ID NO: 29)
DIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGG
SQVQLQQSGPGLVKPSQTLSLTCAISGDSVSSNSAAWNWIRQSPSRGLEWLGRTY
YRSKWYNDYAVSVKSRITINPDTSKNQFSLQLNSVTPEDTAVYYCAREVTGDLE
DAFDIWGQGTMVTVSSGSTSGSGKPGSGEGSTKGDIQMTQSPSSLSASVGDRVTI
TCRASQTIWSYLNWYQQRPGKAPNLLIYAASSLQSGVPSRFSGRGSGTDFTLTISS
LQAEDFATYYCQQSYSIPQTFGQGTKLEIKGGGGSEVKLQESGPGLVAPSQSLSV
TCTVSGVSLPDYGVSWIRQPPRKGLEWLGVIWGSETTYYNSALKSRLTIIKDNSK
SQVFLKMNSLQTDDTAIYYCAKHYYYGGSYAMDYWGQGTSVTVSSSGTTTPAP
RPPTPAPTIASQPLSLRPEACRPAAGGAVHTRGLDFACDIYIWAPLAGTCGVLLLS
LVITLYCKRGRKKLLYIFKQPFMRPVQTTQEEDGCSCRFPEEEEGGCELRVKFSRS
ADAPAYKQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR,
or
iv.
  (SEQ ID NO: 30)
QVQLQQSGPGLVKPSQTLSLTCAISGDSVSSNSAAWNWIRQSPSRGLEWLGRTY
YRSKWYNDYAVSVKSRITINPDTSKNQFSLQLNSVTPEDTAVYYCAREVTGDLE
DAFDIWGQGTMVTVSSGGGGSDIQMTQSPSSLSASVGDRVTITCRASQTIWSYLN
WYQQRPGKAPNLLIYAASSLQSGVPSRFSGRGSGTDFTLTISSLQAEDFATYYCQ
QSYSIPQTFGQGTKLEIKGGGGSGGGGSGGGGSGGGGSGGGGSDIQMTQTTSSLS
ASLGDRVTISCRASQDISKYLNWYQQKPDGTVKLLIYHTSRLHSGVPSRFSGSGS
GTDYSLTISNLEQEDIATYFCQQGNTLPYTFGGGTKLEITGSTSGSGKPGSGEGST
KGEVKLQESGPGLVAPSQSLSVTCTVSGVSLPDYGVSWIRQPPRKGLEWLGVIW
GSETTYYNSALKSRLTIIKDNSKSQVFLKMNSLQTDDTAIYYCAKHYYYGGSYA
MDYWGQGTSVTVSSTTTPAPRPPTPAPTIASQPLSLRPEACRPAAGGAVHTRGLD
FACDIYIWAPLAGTCGVLLLSLVITLYCKRGRKKLLYIFKQPFMRPVQTTQEEDG
CSCRFPEEEEGGCELRVKFSRSADAPAYKQGQNQLYNELNLGRREEYDVLDKRR
GRDPEMGGKPRRKNPQEGLYNELQKDKMAEAYSEIGMKGERRRGKGHDGLYQ
GLSTATKDTYDALHMQALPPR.
```

11. The multi-component cellular therapy product of any one of claims 1-9, wherein:
a) the CAR comprises an antigen-binding domain specific for B-cell maturation antigen (BCMA), wherein the antigen-binding domain comprises a heavy chain variable (VH) region comprising a heavy chain complementarity determining region 1 (CDR-H1) a complementarity determining region 2 (CDR-H2), and a complementarity determining region 3 (CDR-H3), wherein the VH comprises CDR-H1, CDR-H2, and CDR-H3 amino acid sequences selected from the group consisting of:
```
i.
  (SEQ ID NO: 42)
GFTFTNHA,
  (SEQ ID NO: 43)
ISGNGRTT,
and
(SEQ ID NO: 44)
AKDGGETLVDS,
ii.
  (SEQ ID NO: 45)
GFTFSSHA;
  (SEQ ID NO: 46)
ISGSGDFT,
and
(SEQ ID NO: 47)
AKDEDGGSLLGY,
iii
  (SEQ ID NO: 48)
GFTFSSYA;
  (SEQ ID NO: 49)
ISGSGDYI,
and
  (SEQ ID NO: 50)
AKEGTGANSSLADY,
and
iv
  (SEQ ID NO: 51)
GFTFSSHA,
  (SEQ ID NO: 52)
ISGSGDYT,
and
(SEQ ID NO: 53)
AKDEDGGSLLGH,
```
optionally wherein the antigen-binding domain comprises a VH having amino acid sequence selected from the group consisting of:
```
i.
  (SEQ ID NO: 54)
QVQLVESGGGLVQPGGSLRLSCAASGFTFTNHAMSWVRQAPGKGLELVSS
ISGNGRTTYYADSVKGRFTISRDISKNTLDLQMNSLRAEDTAVYYCAKDG
GETLVDSRGQGTLVTVSS,
ii.
  (SEQ ID NO: 55)
QVQLVESGGGLVQPGGSLRLSCAASGFTFSSHAMTWVRQAPGKGLEWVAA
ISGSGDFTHYADSVKGRFTISRDNSKNTVSLQMNNLRAEDTAVYYCAKDE
DGGSLLGYRGQGTLVTVSS,
iii.
  (SEQ ID NO: 56)
EVQLLESGGGLVQPGGSLRLSCAASGFTFSSYAMSWVRQAPGKGLEWVSS
ISGSGDYIYYADSVKGRFTISRDISKNTLYLQMNSLRAEDTAVYYCAKEG
TGANSSLADYRGQGTLVTVSS,
and
iv.
  (SEQ ID NO: 57)
EVQLLESGGGLIQPGGSLRLSCAASGFTFSSHAMTWVRQAPGKGLEWVSA
ISGSGDYTHYADSVKGRFTISRDNSKNTVYLQMNSLRAEDSAVYYCAKDE
DGGSLLGHRGQGTLVTVSS;
```
and/or
b) the CAR comprises an antigen-binding domain specific for B-cell maturation antigen (BCMA), wherein the antigen-binding domain comprises a heavy chain variable (VH) region and a light chain variable (VL) region,
wherein the VH comprises an amino acid sequence selected from the group consisting of:
```
i.
  (SEQ ID NO: 58)
QIQLVQSGPELKKPGETVKISCKASGYTFRHYSMNWVKQAPGKGLKWMGR
INTESGVPIYADDFKGRFAFSVETSASTAYLVINNLKDEDTASYFCSNDY
LYSLDFWGQGTALTVSS,
and
ii.
  (SEQ ID NO: 60)
QIQLVQSGPELKKPGETVKISCKASGYTFTDYSINWVKRAPGKGLKWMGW
INTETREPAYAYDFRGRFAFSLETSASTAYLQINNLKYEDTATYFCALDY
SYAMDYWGQGTSVTVSS,
```
and
wherein the VL comprises an amino acid sequence selected from the group consisting of:
i.
(SEQ ID NO: 59)
DIVLTQSPPSLAMSLGKRATISCRASESVTILGSHLIYWYQQKPGQPPTL
LIQLASNVQTGVPARFSGSGSRTDFTLTIDPVEEDDVAVYYCLQSRTIPR
TFGGGTKLEIK,
ii.
(SEQ ID NO: 61)
DIVLTQSPPSLAMSLGKRATISCRASESVTILGSHLIHWYQQKPGQPPTL
LIQLASNVQTGVPARFSGSGSRTDFTLTIDPVEEDDVAVYYCLQSRTIPR
TFGGGTKLEIK,
iii.
(SEQ ID NO: 62)
DIVLTQSPASLAVSLGQRATISCRASESVDNYGFSFMHWYQQKPGQPPKL
LIYRASNLESGIPARFSGSGSRTDFALTINPVETDDVATYYCQQSNKDPR
TFGGGTKLEIK,
and
iv.
(SEQ ID NO: 63)
DIVLTQSPASLAVSLGQRATISCRASESVDNYGFSFMHWYQQKPGQPPKL
LIYRASNLESGIPARFSGSGSRTDFALTINPVETDDVATYYCQQSNKDPR
TFGGGTKLEIK,
optionally wherein the antigen-binding domain comprises a BCMA scFv having an amino acid sequence selected from the group consisting of:
```
i.
  (SEQ ID NO: 64)
DIVLTQSPPSLAMSLGKRATISCRASESVTILGSHLIYWYQQKPGQPPTL
LIQLASNVQTGVPARFSGSGSRTDFTLTIDPVEEDDVAVYYCLQSRTIPR
TFGGGTKLEIKGSTSGSGKPGSGEGSTKGQIQLVQSGPELKKPGETVKIS
CKASGYTFRHYSMNWVKQAPGKGLKWMGRINTESGVPIYADDFKGRFAFS
VETSASTAYLVINNLKDEDTASYFCSNDYLYSLDFWGQGTALTVSS,
ii.
  (SEQ ID NO: 65)
DIVLTQSPPSLAMSLGKRATISCRASESVTILGSHLIHWYQQKPGQPPTL
LIQLASNVQTGVPARFSGSGSRTDFTLTIDPVEEDDVAVYYCLQSRTIPR
TFGGGTKLEIKGSTSGSGKPGSGEGSTKGQIQLVQSGPELKKPGETVKIS
CKASGYTFTDYSINWVKRAPGKGLKWMGWINTETREPAYAYDFRGRFAFS
LETSASTAYLQINNLKYEDTATYFCALDYSYAMDYWGQGTSVTVSS,
iii
  (SEQ ID NO: 66)
DIVLTQSPASLAVSLGQRATISCRASESVDNYGFSFMHWYQQKPGQPPKL
LIYRASNLESGIPARFSGSGSRTDFALTINPVETDDVATYYCQQSNKDPR
TFGGGTKLEIKGSTSGSGKPGSGEGSTKGQIQLVQSGPELKKPGETVKIS
CKASGYTFTDYSINWVKRAPGKGLKWMGWINTETREPAYAYDFRGRFAFS
LETSASTAYLQINNLKYEDTATYFCALDYSYAMDYWGQGTSVTVSS,
and
iv.
  (SEQ ID NO: 67)
DIVLTQSPASLAMSLGKRATISCRASESVSVIGAHLIHWYQQKPGQPPKL
LIYLASNLETGVPARFSGSGSGTDFTLTIDPVEEDDVAIYSCLQSRIFPR
TFGGGTKLEIKGSTSGSGKPGSGEGSTKGQIQLVQSGPELKKPGETVKIS
CKASGYTFTDYSINWVKRAPGKGLKWMGWINTETREPAYAYDFRGRFAFS
LETSASTAYLQINNLKYEDTATYFCALDYSYAMDYWGQGTSVTVSS;
```
and/or
c) the CAR comprises one or more single-domain binding regions specific for B-cell maturation antigen (BCMA), wherein each single-domain binding region comprises an amino acid sequence selected from the group consisting of:
```
i.
  (SEQ ID NO: 68)
VKLEESGGGLVQAGRSLRLSCAASEHTFSSHVMGWFRQAPGKERESVAVI
GWRDISTSYADSVKGRFTISRDNAKKTLYLQMNSLKPEDTAVYYCAARRI
DAADFDSWGQGTQVTVSS,
and
ii.
  (SEQ ID NO: 69)
EVQLVESGGGLVQAGGSLRLSCAASGRTFTMGWFRQAPGKEREFVAAISL
SPTLAYYAESVKGRFTISRDNAKNTVVLQMNSLKPEDTALYYCAADRKSV
MSIRPDYWGQGTQVTVSS;
```
and/or
d) the CAR comprises an antigen-binding domain specific for B-cell maturation antigen (BCMA), wherein the binding domain comprises the amino acid sequence of
(SEQ ID NO: 70)
MGSWSEFWVRLGAIRERLDALGGSEAELAAFEKEIAAFESELQAYKGKGN
PEVEKLRYTAATIRRFLQAYRHN;
and/or
e) the CAR comprises an amino acid sequence selected from the group consisting of:
```
i.
  (SEQ ID NO: 71)
MALPVTALLLPLALLLHAARPQVQLVESGGGLVQPGGSLRLSCAASGFTFTNH
AMSWVRQAPGKGLELVSSISGNGRTTYYADSVKGRFTISRDISKNTLDLQMNS
LRAEDTAVYYCAKDGGETLVDSRGQGTLVTVSSFVPVFLPAKPTTTPAPRPPTP
APTIASQPLSLRPEACRPAAGGAVHTRGLDFACDIYIWAPLAGTCGVLLLSLVIT
LYCNHRNRSKRSRLLHSDYMNMTPRRPGPTRKHYQPYAPPRDFAAYRSRVKF
SRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHM
QALPPR;
ii.
  (SEQ ID NO: 72)
MALPVTALLLPLALLLHAARPQVQLVESGGGLVQPGGSLRLSCAASGFTFSSH
AMTWVRQAPGKGLEWVAAISGSGDFTHYADSVKGRFTISRDNSKNTVSLQMN
NLRAEDTAVYYCAKDEDGGSLLGYRGQGTLVTVSSFVPVFLPAKPTTTPAPRP
PTPAPTIASQPLSLRPEACRPAAGGAVHTRGLDFACDIYIWAPLAGTCGVLLLS
LVITLYCNHRNRSKRSRLLHSDYMNMTPRRPGPTRKHYQPYAPPRDFAAYRSR
VKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDA
LHMQALPPR;
iii.
  (SEQ ID NO: 73)
MALPVTALLLPLALLLHAARPEVQLLESGGGLVQPGGSLRLSCAASGFTFSSYA
MSWVRQAPGKGLEWVSSISGSGDYIYYADSVKGRFTISRDISKNTLYLQMNSL
RAEDTAVYYCAKEGTGANSSLADYRGQGTLVTVSSFVPVFLPAKPTTTPAPRP
PTPAPTIASQPLSLRPEACRPAAGGAVHTRGLDFACDIYIWAPLAGTCGVLLLS
LVITLYCNHRNRSKRSRLLHSDYMNMTPRRPGPTRKHYQPYAPPRDFAAYRSR
VKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDA
LHMQALPPR;
iv.
  (SEQ ID NO: 74)
MALPVTALLLPLALLLHAARPEVQLLESGGGLIQPGGSLRLSCAASGFTFSSHA
MTWVRQAPGKGLEWVSAISGSGDYTHYADSVKGRFTISRDNSKNTVYLQMNS
LRAEDSAVYYCAKDEDGGSLLGHRGQGTLVTVSSFVPVFLPAKPTTTPAPRPP
TPAPTIASQPLSLRPEACRPAAGGAVHTRGLDFACDIYIWAPLAGTCGVLLLSL
VITLYCNHRNRSKRSRLLHSDYMNMTPRRPGPTRKHYQPYAPPRDFAAYRSRV
KFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDAL
HMQALPPR;
V.
  (SEQ ID NO: 75)
MALPVTALLLPLALLLHAARPQVQLVESGGGLVQPGGSLRLSCAASGFTFTNH
AMSWVRQAPGKGLELVSSISGNGRTTYYADSVKGRFTISRDISKNTLDLQMNS
LRAEDTAVYYCAKDGGETLVDSRGQGTLVTVSSFVPVFLPAKPTTTPAPRPPTP
APTIASQPLSLRPEACRPAAGGAVHTRGLDFACDIYIWAPLAGTCGVLLLSLVIT
LYCNHRNKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHM
QALPPR;
vi.
  (SEQ ID NO: 76)
MALPVTALLLPLALLLHAARPQVQLVESGGGLVQPGGSLRLSCAASGFTFSSH
AMTWVRQAPGKGLEWVAAISGSGDFTHYADSVKGRFTISRDNSKNTVSLQMN
NLRAEDTAVYYCAKDEDGGSLLGYRGQGTLVTVSSFVPVFLPAKPTTTPAPRP
PTPAPTIASQPLSLRPEACRPAAGGAVHTRGLDFACDIYIWAPLAGTCGVLLLS
LVITLYCNHRNKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDA
LHMQALPPR;
vii.
  (SEQ ID NO: 77)
MALPVTALLLPLALLLHAARPEVQLLESGGGLVQPGGSLRLSCAASGFTFSSYA
MSWVRQAPGKGLEWVSSISGSGDYIYYADSVKGRFTISRDISKNTLYLQMNSL
RAEDTAVYYCAKEGTGANSSLADYRGQGTLVTVSSFVPVFLPAKPTTTPAPRP
PTPAPTIASQPLSLRPEACRPAAGGAVHTRGLDFACDIYIWAPLAGTCGVLLLS
LVITLYCNHRNKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDA
LHMQALPPR;
viii.
  (SEQ ID NO: 78)
MALPVTALLLPLALLLHAARPEVQLLESGGGLIQPGGSLRLSCAASGFTFSSHA
MTWVRQAPGKGLEWVSAISGSGDYTHYADSVKGRFTISRDNSKNTVYLQMNS
LRAEDSAVYYCAKDEDGGSLLGHRGQGTLVTVSSFVPVFLPAKPTTTPAPRPP
TPAPTIASQPLSLRPEACRPAAGGAVHTRGLDFACDIYIWAPLAGTCGVLLLSL
VITLYCNHRNKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDAL
HMQALPPR;
ix.
  (SEQ ID NO: 79)
MALPVTALLLPLALLLHAARPQVQLVESGGGLVQPGGSLRLSCAASGFTFTNH
AMSWVRQAPGKGLELVSSISGNGRTTYYADSVKGRFTISRDISKNTLDLQMNS
LRAEDTAVYYCAKDGGETLVDSRGQGTLVTVSSFVPVFLPAKPTTTPAPRPPTP
APTIASQPLSLRPEACRPAAGGAVHTRGLDFACDIYIWAPLAGTCGVLLLSLVIT
LYCNHRNCWLTKKKYSSSVHDPNGEYMFMRAVNTAKKSRLTDVTLRVKFSR
SADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQA
LPPR;
x.
  (SEQ ID NO: 80)
MALPVTALLLPLALLLHAARPQVQLVESGGGLVQPGGSLRLSCAASGFTFSSH
AMTWVRQAPGKGLEWVAAISGSGDFTHYADSVKGRFTISRDNSKNTVSLQMN
NLRAEDTAVYYCAKDEDGGSLLGYRGQGTLVTVSSFVPVFLPAKPTTTPAPRP
PTPAPTIASQPLSLRPEACRPAAGGAVHTRGLDFACDIYIWAPLAGTCGVLLLS
LVITLYCNHRNCWLTKKKYSSSVHDPNGEYMFMRAVNTAKKSRLTDVTLRV
KFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDAL
HMQALPPR;
xi.
  (SEQ ID NO: 81)
MALPVTALLLPLALLLHAARPEVQLLESGGGLVQPGGSLRLSCAASGFTFSSYA
MSWVRQAPGKGLEWVSSISGSGDYIYYADSVKGRFTISRDISKNTLYLQMNSL
RAEDTAVYYCAKEGTGANSSLADYRGQGTLVTVSSFVPVFLPAKPTTTPAPRP
PTPAPTIASQPLSLRPEACRPAAGGAVHTRGLDFACDIYIWAPLAGTCGVLLLS
LVITLYCNHRNCWLTKKKYSSSVHDPNGEYMFMRAVNTAKKSRLTDVTLRV
KFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDAL
HMQALPPR;
xii.
  (SEQ ID NO: 82)
MALPVTALLLPLALLLHAARPEVQLLESGGGLIQPGGSLRLSCAASGFTFSSHA
MTWVRQAPGKGLEWVSAISGSGDYTHYADSVKGRFTISRDNSKNTVYLQMNS
LRAEDSAVYYCAKDEDGGSLLGHRGQGTLVTVSSFVPVFLPAKPTTTPAPRPP
TPAPTIASQPLSLRPEACRPAAGGAVHTRGLDFACDIYIWAPLAGTCGVLLLSL
VITLYCNHRNCWLTKKKYSSSVHDPNGEYMFMRAVNTAKKSRLTDVTLRVKF
SRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHM
QALPPR;
xiii.
  (SEQ ID NO: 83);
MALPVTALLLPLALLLHAARPDIVLTQSPPSLAMSLGKRATISCRASESVTILGS
HLIYWYQQKPGQPPTLLIQLASNVQTGVPARFSGSGSRTDFTLTIDPVEEDDVA
VYYCLQSRTIPRTFGGGTKLEIKGSTSGSGKPGSGEGSTKGQIQLVQSGPELKKP
GETVKISCKASGYTFRHYSMNWVKQAPGKGLKWMGRINTESGVPIYADDFKG
RFAFSVETSASTAYLVINNLKDEDTASYFCSNDYLYSLDFWGQGTALTVSSFVP
VFLPAKPTTTPAPRPPTPAPTIASQPLSLRPEACRPAAGGAVHTRGLDFACDIYI
WAPLAGTCGVLLLSLVITLYCNHRNRSKRSRLLHSDYMNMTPRRPGPTRKHY
QPYAPPRDFAAYRSRVKFSRSADAPAYQQGQNQLYNELNLGRREEYDVLDKR
RGRDPEMGGKPRRKNPQEGLYNELQKDKMAEAYSEIGMKGERRRGKGHDGL
YQGLSTATKDTYDALHMQALPPR
xiv.
  (SEQ ID NO: 84)
MALPVTALLLPLALLLHAARPDIVLTQSPPSLAMSLGKRATISCRASESVTILGS
HLIHWYQQKPGQPPTLLIQLASNVQTGVPARFSGSGSRTDFTLTIDPVEEDDVA
VYYCLQSRTIPRTFGGGTKLEIKGSTSGSGKPGSGEGSTKGQIQLVQSGPELKKP
GETVKISCKASGYTFTDYSINWVKRAPGKGLKWMGWINTETREPAYAYDFRG
RFAFSLETSASTAYLQINNLKYEDTATYFCALDYSYAMDYWGQGTSVTVSSFV
PVFLPAKPTTTPAPRPPTPAPTIASQPLSLRPEACRPAAGGAVHTRGLDFACDIYI
WAPLAGTCGVLLLSLVITLYCNHRNRSKRSRLLHSDYMNMTPRRPGPTRKHY
QPYAPPRDFAAYRSRVKFSRSADAPAYQQGQNQLYNELNLGRREEYDVLDKR
RGRDPEMGGKPRRKNPQEGLYNELQKDKMAEAYSEIGMKGERRRGKGHDGL
YQGLSTATKDTYDALHMQALPPR;
xv.
  (SEQ ID NO: 85)
MALPVTALLLPLALLLHAARPDIVLTQSPPSLAMSLGKRATISCRASESVTILGS
HLIYWYQQKPGQPPTLLIQLASNVQTGVPARFSGSGSRTDFTLTIDPVEEDDVA
VYYCLQSRTIPRTFGGGTKLEIKGSTSGSGKPGSGEGSTKGQIQLVQSGPELKKP
GETVKISCKASGYTFTHYSMNWVKQAPGKGLKWMGRINTETGEPLYADDFK
GRFAFSLETSASTAYLVINNLKNEDTATFFCSNDYLYSCDYWGQGTTLTVSSFV
PVFLPAKPTTTPAPRPPTPAPTIASQPLSLRPEACRPAAGGAVHTRGLDFACDIYI
WAPLAGTCGVLLLSLVITLYCNHRNRSKRSRLLHSDYMNMTPRRPGPTRKHY
QPYAPPRDFAAYRSRVKFSRSADAPAYQQGQNQLYNELNLGRREEYDVLDKR
RGRDPEMGGKPRRKNPQEGLYNELQKDKMAEAYSEIGMKGERRRGKGHDGL
YQGLSTATKDTYDALHMQALPPR;
xvi.
  (SEQ ID NO: 86)
MLLLVTSLLLCELPHPAFLLIPDIVLTQSPPSLAMSLGKRATISCRASESVTILGS
HLIHWYQQKPGQPPTLLIQLASNVQTGVPARFSGSGSRTDFTLTIDPVEEDDVA
VYYCLQSRTIPRTFGGGTKLEIKGSTSGSGKPGSGEGSTKGQIQLVQSGPELKKP
GETVKISCKASGYTFTDYSINWVKRAPGKGLKWMGWINTETREPAYAYDFRG
RFAFSLETSASTAYLQINNLKYEDTATYFCALDYSYAMDYWGQGTSVTVSSA
AAFVPVFLPAKPTTTPAPRPPTPAPTIASQPLSLRPEACRPAAGGAVHTRGLDFA
CDIYIWAPLAGTCGVLLLSLVITLYCNHRNRSKRSRLLHSDYMNMTPRRPGPT
RKHYQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRGKG
HDGLYQGLSTATKDTYDALHMQALPPR;
xvii.
  (SEQ ID NO: 87)
MALPVTALLLPLALLLHAARPDIVLTQSPPSLAMSLGKRATISCRASESVTILGS
HLIHWYQQKPGQPPTLLIQLASNVQTGVPARFSGSGSRTDFTLTIDPVEEDDVA
VYYCLQSRTIPRTFGGGTKLEIKGSTSGSGKPGSGEGSTKGQIQLVQSGPELKKP
GETVKISCKASGYTFTDYSINWVKRAPGKGLKWMGWINTETREPAYAYDFRG
RFAFSLETSASTAYLQINNLKYEDTATYFCALDYSYAMDYWGQGTSVTVSSA
AAFVPVFLPAKPTTTPAPRPPTPAPTIASQPLSLRPEACRPAAGGAVHTRGLDFA
CDIYIWAPLAGTCGVLLLSLVITLYCNHRNRSKRSRLLHSDYMNMTPRRPGPT
RKHYQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRGKG
HDGLYQGLSTATKDTYDALHMQALPPR;
xviii.
  (SEQ ID NO: 88)
MALPVTALLLPLALLLHAARPDIVLTQSPPSLAMSLGKRATISCRASESVTILGS
HLIHWYQQKPGQPPTLLIQLASNVQTGVPARFSGSGSRTDFTLTIDPVEEDDVA
VYYCLQSRTIPRTFGGGTKLEIKGSTSGSGKPGSGEGSTKGQIQLVQSGPELKKP
GETVKISCKASGYTFTDYSINWVKRAPGKGLKWMGWINTETREPAYAYDFRG
RFAFSLETSASTAYLQINNLKYEDTATYFCALDYSYAMDYWGQGTSVTVSSA
AAFVPVFLPAKPTTTPAPRPPTPAPTIASQPLSLRPEACRPAAGGAVHTRGLDFA
CDIYIWAPLAGTCGVLLLSLVITLYCNHRNRFSVVKRGRKKLLYIFKQPFMRPV
QTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERR
RGKGHDGLYQGLSTATKDTYDALHMQALPPR;
xix.
  (SEQ ID NO: 89)
MALPVTALLLPLALLLHAARPDIVLTQSPPSLAMSLGKRATISCRASESVTILGS
HLIHWYQQKPGQPPTLLIQLASNVQTGVPARFSGSGSRTDFTLTIDPVEEDDVA
VYYCLQSRTIPRTFGGGTKLEIKGSTSGSGKPGSGEGSTKGQIQLVQSGPELKKP
GETVKISCKASGYTFTDYSINWVKRAPGKGLKWMGWINTETREPAYAYDFRG
RFAFSLETSASTAYLQINNLKYEDTATYFCALDYSYAMDYWGQGTSVTVSSA
AAFVPVFLPAKPTTTPAPRPPTPAPTIASQPLSLRPEACRPAAGGAVHTRGLDFA
CDIYIWAPLAGTCGVLLLSLVITLYCNHRNRRDQRLPPDAHKPPGGGSFRTPIQ
EEQADAHSTLAKIRVKFSRSADAPAYQQGQNQLYNELNLGRREEYDVLDKRR
GRDPEMGGKPRRKNPQEGLYNELQKDKMAEAYSEIGMKGERRRGKGHDGLY
QGLSTATKDTYDALHMQALPPR;
xx.
  (SEQ ID NO: 90)
MALPVTALLLPLALLLHAARPDIVLTQSPPSLAMSLGKRATISCRASESVTILGS
HLIHWYQQKPGQPPTLLIQLASNVQTGVPARFSGSGSRTDFTLTIDPVEEDDVA
VYYCLQSRTIPRTFGGGTKLEIKGSTSGSGKPGSGEGSTKGQIQLVQSGPELKKP
GETVKISCKASGYTFTDYSINWVKRAPGKGLKWMGWINTETREPAYAYDFRG
RFAFSLETSASTAYLQINNLKYEDTATYFCALDYSYAMDYWGQGTSVTVSSA
AAFVPVFLPAKPTTTPAPRPPTPAPTIASQPLSLRPEACRPAAGGAVHTRGLDFA
CDIYIWAPLAGTCGVLLLSLVITLYCNHRNRFSVVKRGRKKLLYIFKQPFMRPV
QTTQEEDGCSCRFPEEEEGGCELRRDQRLPPDAHKPPGGGSFRTPIQEEQADAH
STLAKIRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTAT
KDTYDALHMQALPPR;
xxi.
  (SEQ ID NO: 91)
MALPVTALLLPLALLLHAARPQVKLEESGGGLVQAGRSLRLSCAASEHTFSSH
VMGWFRQAPGKERESVAVIGWRDISTSYADSVKGRFTISRDNAKKTLYLQMN
SLKPEDTAVYYCAARRIDAADFDSWGQGTQVTVSSGGGGSEVQLVESGGGLV
QAGGSLRLSCAASGRTFTMGWFRQAPGKEREFVAAISLSPTLAYYAESVKGRF
TISRDNAKNTVVLQMNSLKPEDTALYYCAADRKSVMSIRPDYWGQGTQVTVS
STSTTTPAPRPPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEE
GGCELRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTAT
KDTYDALHMQALPPR;
and
xxii.
  (SEQ ID NO: 92)
MAFLWLLSCWALLGTTFGDYKDDDDKGGGGSGGGGSMGSWSEFWVRLGAI
RERLDALGGSEAELAAFEKEIAAFESELQAYKGKGNPEVEKLRYTAATIRRFLQ
AYRHNGGGGSGGGGSGTTTPAPRPPTPAPTIASQPLSLRPEACRPAAGGAVHTR
GLDFACDIYIWAPLAGTCGVLLLSLVITLYCKRGRKKLLYIFKQPFMRPVQTTQ
EEDGCSCRFPEEEEGGCELRVKFSRSADAPAYKQGQNQLYNELNLGRREEYDV
LDKRRGRDPEMGGKPRRKNPQEGLYNELQKDKMAEAYSEIGMKGERRRGKG
HDGLYQGLSTATKDTYDALHMQALPPR.
```

12. The multi-component cellular therapy product of any one of claims 1-11, wherein:
a) the product comprises a dose of CAR⁺ cells that ranges from approximately 1.0×10⁵ to approximately 5.0×10⁶ CAR⁺ cells per kilogram of body weight of a human subject receiving the product, or from approximately 4.0×10⁵ to 1.0×10⁹ CAR⁺ cells; and/or
b) the one or more cells comprising the chimeric receptor or the pharmaceutical composition are selected from the group consisting of: T cells, Natural Killer (NK) cells, cytotoxic T lymphocytes (CTLs), regulatory T cells, and Natural Killer T (NKT) cells; and/or
c) the one or more cells comprising the chimeric receptor or the pharmaceutical composition are from an allogeneic donor and have been genetically modified to reduce risk of graft versus host disease (GVHD) and/or to prevent allogeneic rejection.

13. A pharmaceutical composition comprising the multi-component cellular therapy product of any one of claims 1-12.

14. A method of treating a human subject having or suspected of having a malignancy, comprising administering to the human subject the multi-component cellular therapy product of any one of claims 1-12 or the pharmaceutical composition of claim 13,
optionally wherein the hematologic malignancy is selected from the group consisting of: acute lymphoblastic leukemia (ALL), acute myelogenous leukemia (AML), chronic myelogenous leukemia (CML), multiple myeloma, lymphoma, Hodgkin's lymphoma, non-Hodgkin lymphoma, myelodysplastic syndrome, myelofibrosis, and blastic plasmacytoid dendritic cell neoplasm (BPDCN).

15. The multi-component cellular therapy product of any one of claims 1-12, or the pharmaceutical composition of claim 13 for use in a method of treating a human subject having or suspected of having a malignancy,
optionally wherein the hematologic malignancy is selected from the group consisting of: acute lymphoblastic leukemia (ALL), acute myelogenous leukemia (AML), chronic myelogenous leukemia (CML), multiple myeloma, lymphoma, Hodgkin's lymphoma, non-Hodgkin lymphoma, myelodysplastic syndrome, myelofibrosis, and blastic plasmacytoid dendritic cell neoplasm (BPDCN).

16. An engineered cellular graft comprising the multi-component cellular therapy product of any one of claims 1-12.

17. A cellular therapy kit comprising the multi-component cellular therapy product of any one of claims 1-12 or the pharmaceutical composition of claim 13,
optionally wherein the kit further comprises written instructions for using the cellular therapy for treating a hematologic malignancy in a human subject.

18. A kit for use in preparation of a cellular therapy product, the kit comprising:
(a) an anti-human CD34 affinity reagent;
(b) an anti-human CD25 affinity reagent;
(c) a vector comprising a nucleic acid encoding a chimeric receptor; and
(d) a transfection reagent,
optionally wherein the kit further comprises written instructions for using the anti-human CD34 affinity reagent to isolate HSPCs from a donor blood sample or a donor bone marrow sample,
optionally wherein the written instructions further comprise instructions for using the anti-human CD25 affinity reagent to isolate Tregs from the donor blood sample or the donor bone marrow sample,
optionally wherein the written instructions further comprise instructions for using the transfection reagent to introduce the vector into one or more cells from the donor blood sample or the donor bone marrow sample.

19. A method of preparing a multi-component cellular therapy product comprising:
a) obtaining one or more peripheral blood samples or bone marrow samples from a donor that is allogeneic with respect to a human subject receiving the product;
b) collecting a first population of cells from the one or more peripheral blood samples or bone marrow samples and transfecting the population of cells with a vector comprising a nucleic acid encoding the chimeric receptor of any one of claims 1-12;
c) combining the transfected population of cells with one or more cryoprotectants and cryopreserving the transfected population of cells;
d) collecting a second population of cells from the one or more peripheral blood samples or bone marrow samples and combining the second population of cells with one or more cryoprotectants and cryopreserving the second population of cells,
wherein the second population of cells comprises conventional CD3⁺ T cells (Tcons);
e) sorting a third population of cells from the one or more peripheral blood samples or bone marrow samples and with one or more immune-separation particles (ISPs) specific for CD34 to obtain a CD34-enriched cell population and a CD34-depleted cell population,
wherein the CD34-enriched cell population comprises CD34⁺ hematopoietic stem and progenitor cells (HSPCs);
f) sorting the CD34-depleted cell population with one or more ISPs specific for CD25 to obtain a CD25-enriched cell population and a CD25-depleted cell population; and
g) sorting the CD25-enriched cell population with one or more ISPs specific for CD4 and one or more ISPs specific for CD127, and selecting a CD4⁺ and CD127^{dim} cell population, wherein the CD4⁺ and CD127^{dim} cell population comprises CD4⁺CD25⁺CD127^{dim} regulatory T cells (Tregs),
wherein the multi-component cellular therapy product comprises the transfected cell population, the Tcons, the HSPCs, and the Tregs,
optionally wherein the first population of cells comprises the Tcons or the first population of cells comprises the HSPCs,
optionally wherein the CD4⁺ and CD127^{dim} cell population is the population of cells transfected with the vector.
